(19) United States
(12) Patent Application Publication
(10) Pub. No.: US 2014/0170151 A1
(43) Pub. Date: Jun. 19, 2014

(54) Synthetic Oligosaccharides for Staphylococcus Vaccine

(71) Applicants: A. Stewart CAMPBELL, Framingham, MA (US); Obadiah J. PLANTE, Danvers, MA (US)

(72) Inventors: A. Stewart CAMPBELL, Framingham, MA (US); Obadiah J. PLANTE, Danvers, MA (US)

(21) Appl. No.: 14/177,590

(22) Filed: Feb. 11, 2014

Related U.S. Application Data
(63) Continuation of application No. 13/642,175, filed on Jan. 11, 2013, now abandoned.
(60) Provisional application No. 61/327,626, filed on Apr. 23, 2010.

(51) Int. Cl. C07K 17/10

(52) U.S. Cl. CPC C07K 17/10
USPC 424/137.1; 536/55.1; 530/405; 424/194.1; 530/387.5

(57) ABSTRACT

The present invention synthetic oligo-β-(1→6)-glucosamine structures and a methodology which essentially allows for the synthesis of any oligo-β-(1→6)-glucosamine species having a definite number of monosaccharide units, including a set pattern of acetylated and non-acetylated residues. The invention further provides antibodies to these synthetic oligo-β-(1→6)-glucosamines as well as compositions thereof and methods for treating and preventing infections caused by bacteria expressing poly-β-(1→6)-glucosamines, such as Staphylococcus aureus.

### CROSS-REFERENCE TO RELATED APPLICATIONS

This application is a continuation of U.S. application Ser. No. 13/642,175, which is a national stage application of International Application No. PCT/US2011/000723, filed Apr. 22, 2011, which claims the benefit of U.S. Provisional Application No. 61/327,626, filed Apr. 23, 2010. The entire contents of the aforementioned applications are hereby incorporated herein by reference.

### FIELD OF THE INVENTION

The present invention relates to synthetic oligo-β-(1→6)-glucosamine structures exhibiting a defined substitution pattern.

### BACKGROUND

Staphylococcal infection can lead to a wide range of severe clinical disease manifestations and represents a major public healthcare concern. The cell walls of Staphylococci contain a diverse array of glycopeptides and glycolipids that serve both structural and virulence functions. The cell wall is comprised primarily of peptidoglycan, a peptide crosslinked polymer of poly-N-acetylglucosamine (PNAG), N-acetylmuramic acid, and glycolipids, including wall teichoic acids (WTA) and lipoteichoic acid (LTA).

Cell wall related factors possess many unique features making them particularly attractive therapeutic targets. Importantly, cell wall components are expressed in the initial colonization, local infection and systemic stages of disease. Unfortunately, a lack of consistent access to material, along with toxicity issues hinder cell-wall targeted vaccine development. For example, LTA is toxic and could not be used as the basis of a vaccine candidate without modification.

Recently, the properties of a cell wall associated polysaccharide, poly-1,6-N-acetylglucosamine (PNAG), also known as polysaccharide intercellular adhesion molecule (PIA), have been described. PNAG serves several key biological functions at various stages of the bacterial infection cycle including adhesion to bacterial and host surfaces, promotion of biofilm formation and protection against antibody-independent opsonic killing. The PNAG molecule and its derivatives are able to mediate all these functions due to a diverse distribution pattern of amine and acetylation modifications to the core structure (FIG. 1). The core structure is comprised of a glucosamine backbone which is only generated by bacterial pathogens. The backbone structure is modified with acetyl or amine groups with variable spacing.

The PNAG molecule has been used in vaccine studies (See Pier et al., U.S. Publication 2005/0118198). Data generated using purified PNAG-based material demonstrates the viability of this carbohydrate-based vaccine approach (Maira-Litran et al., Infect. Immun., 73:6752, 2005). However, in spite of the aforementioned PNAG-focused studies revealing that functional protection and opsonization activity is dependent on the presence of an amine-rich modified PNAG structure (<50% acetylated positions), the major component of biofilms is secreted PNAG that contains mostly (>95%) N-acetylated positions. Accordingly, carbohydrate-based vaccine development is in need of a better understanding of the requirements for maintaining an appropriate acetylation-amine balance in lead PNAG-based vaccine target selection.

A methodology for preparing synthetic PNAGs was described (Gening et al., Infect. Immun., 78:764, 2010, epub Nov. 30, 2009; WO 2010/011284 A2). The described synthetic PNAGs were limited to homogenous PNAG compositions that are fully acetylated or fully non-acetylated; neither of these references, nor those cited therein, described a methodology for synthesizing homogenous mixed PNAGs having a predetermined number and predetermined arrangement of acetylated and deacetylated residues. Moreover, although the previous purification-chemical process utilizing purified PNAG material that was subsequently chemically treated to yield an appropriate range of N-acetylated versus free amine positions on the PNAG molecule (Maira-Litran et al., supra), the purification-chemical process is only able to provide a range of heterogeneous material and therefore only average numbers on the degree of acetylation are available. Moreover, there is no information on the position of the acetylated residues. Accordingly, the prior art processes failed to provide oligosaccharides with spatially defined acetyl-amine positions, such as an amine every third position on the glucosamine polymer, for example. As such, the identification of a precise acetyl-amine sequence required to generate a desired immune response can neither be achieved by this method nor predicted a priori.

In view of the above, the present invention provides several benefits for vaccine development, especially against S. aureus, including production of homogenous antigen compositions with mixed acetyl/amine positions at high purity and at robust levels without contaminating carbohydrate structures that are an almost inevitable consequence of isolation from biological mixtures.

### SUMMARY

The present invention provides oligosaccharides (oligo-β-(1→6)-glucosamine structures) 1a

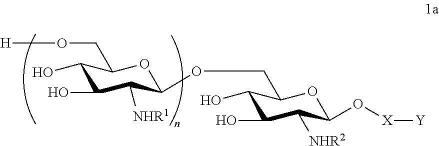

where R¹ and R² are each independently selected from H or C(O)CH₃, where at least one R¹ or R² in the oligosaccharide is H and at least another is C(O)CH₃; n is an integer of at least 3, X is a linker, and Y is H or a carrier; and wherein each occurrence R¹ can be the same or different.

The present invention provides compositions and methods for synthesizing oligo-β-(1→6)-glucosamine structures and conjugates that have a specific number of monosaccharide units and a fixed, defined pattern of acetylated and non-acetylated residues.

The present invention further provides immunogenic and immunoprotective compositions containing synthetic oligo-β-(1→6)-glucosamines 1a and antibodies derived therefrom for diagnosing, treating, and preventing infections caused by bacteria such as Staphylococcus aureus and others.

The present invention further provides a method for chemoselectively deprotecting one of at least two different nitrogen protecting groups in an aminosugar, in particular in an oligo-β-(1→6)-glucosamine.

### BRIEF DESCRIPTION OF THE DRAWINGS

FIG. 1 depicts an exemplary core structure of an oligo-β-(1→6)-glucosamine comprised of a glucosamine backbone unique to bacterial pathogens. The backbone structure can be modified with N-acetyl groups with variable spacing.

FIG. 2A depicts naturally-derived PNAG polysaccharides and FIG. 2B depicts a synthetic PNAG oligosaccharide, including potential conjugation sites in FIGS. 2A and 2B.

FIG. 3 depicts a reaction scheme for synthesizing four monosaccharide building blocks which can be used to synthesize any dPNAG/PNAG oligosaccharide.

FIGS. 4A-4D depict a series of reaction schemes for assembling disaccharide building blocks from the four monosaccharide building blocks depicted in FIG. 3.

FIGS. 5A and 5B depict a reaction scheme for synthesizing a 6-mer thiol oligosaccharide 37.

FIGS. 6A and 6B depict a reaction scheme for synthesizing a 12-mer thiol oligosaccharide 40.

FIG. 7 depicts a reaction scheme for synthesizing an 18-mer thiol oligosaccharide 34.

FIGS. 8A and 8B depict a reaction scheme for conjugating the 6-mer thiol oligosaccharide 37 to BSA (A) or KLH (B) to form a 6-Mix-BSA conjugate 41 or a 6-Mix-KLH conjugate 42, respectively. In the nomenclature used herein, e.g. “6-Mix,” “12-NH₂” and “18-NHAc,” the number refers to the number of monosaccharide units, “Mix” refers to an oligosaccharide with a fixed pattern of both acetyl and amine groups at R¹ and R², “NH₂” refers to an oligosaccharide with amine groups at R¹ and R², and “NHAc” refers to an oligosaccharide with acetyl groups at R¹ and R².

FIGS. 9A and 9B depict a reaction scheme for conjugating the 12-mer thiol oligosaccharide 40 to BSA (A) or KLH (B) to form a 12-Mix-BSA conjugate 43 or 12-Mix-KLH conjugate 44, respectively.

FIGS. 10A and 10B depict a reaction scheme for conjugating the 18-mer thiol oligosaccharide 34 to BSA (A) or KLH (B) to form an 18-Mix-BSA conjugate 45 or 18-Mix-KLH conjugate 46, respectively.

FIGS. 11A-11C depict IgG antibody titers as a function of antibody-antigen complex absorption (OD₄₅₀) at the indicated serum dilutions obtained from 3 succesive bleeds (pre-immune, 1^{st} bleed, and final bleed) in rabbits immunized with KLH conjugates corresponding to: (A) 6-Mix-KLH 42; (B) 12-Mix-KLH 44; and (C) 18-Mix-KLH 46. In each case, the antisera were incubated on ELISA plates adsorbed with their corresponding BSA conjugate, specifically, (A) 6-Mix-BSA 41; (B) 12-Mix-BSA 43; and (C) 18-Mix-BSA 45.

FIGS. 11D-11F depict IgG antibody titers from antigen-specific antigen-KLH conjugate-derived antibodies recovered at three successive stages of affinity purification, including pre-affinity purification (3^{rd} bleed), the flow-through fraction and the antibody-enriched (purified) fraction from rabbits immunized with KLH conjugates. Results are shown as a function of antibody-antigen complex absorption (OD₄₅₀) at the indicated serum dilutions obtained from the above-described antibody-enriched fractions generated against antigen-KLH conjugates corresponding to: (A) 6-Mix-KLH 42; (B) 12-Mix-KLH 44; and (C) 18-Mix-KLH 46. In each case, the antisera were incubated on ELISA plates adsorbed with their corresponding BSA conjugate, specifically, (A) 6-Mix-BSA 41; (B) 12-Mix-BSA 43; and (C) 18-Mix-BSA 45.

FIGS. 12A-12G depict the results of a cross-ELISA assay examining the specificity and cross-reactivity between fully non-acetylated (6-NH₂, 12-NH₂, 18-NH₂); mixed (6-Mix, 12-Mix, 18-Mix) and fully acetylated (6-NHAc, 12-NHAc, 18-NHAc) oligosaccharides 1a and antibodies derived therefrom. In FIGS. 12A-12G, antisera from rabbits immunized with the indicated antigen-KLH conjugates corresponding to (left to right) 6-NH₂, 6-Mix, 6-NHAc, 12-NH₂, 12-Mix, 12-NHAc, 18-NH₂, 18-Mix, and 18-NHAc were incubated in each case with an ELISA plate adsorbed with a different antigen-BSA conjugate, specifically: (A) 6-NH₂—BSA; (B) 6-Mix-BSA; (C) 6-NHAc—BSA; (D) 12-NH₂—BSA; (E) 12-Mix-BSA; (F) 12-NHAc—BSA; (G) 18-Mix-BSA. Results are shown as a function of antibody-antigen complex absorption (OD₄₅₀) representing the averages from two rabbit antiseras in each case at the indicated serum dilutions, whereby total OD₄₅₀ is measured by subtracting away the background OD₄₅₀ from KLH antibodies alone.

FIGS. 13A-13D depict the results of a whole-cell ELISA assay examining the binding of pre-immune sera (A, C) or immune sera (B, D) generated from rabbits immunized against (left to right) KLH control, non-acetylated (12-NH₂); mixed (12-Mix) and non-acetylated (12-NHAc) oligo-β-(1→6)-glucosamines and S. epidermidis coated onto ELISA fixed with methanol (A, B) or formalin (C, D). Results are shown as a function of antibody-antigen complex absorption (OD₄₅₀) at the indicated serum dilutions.

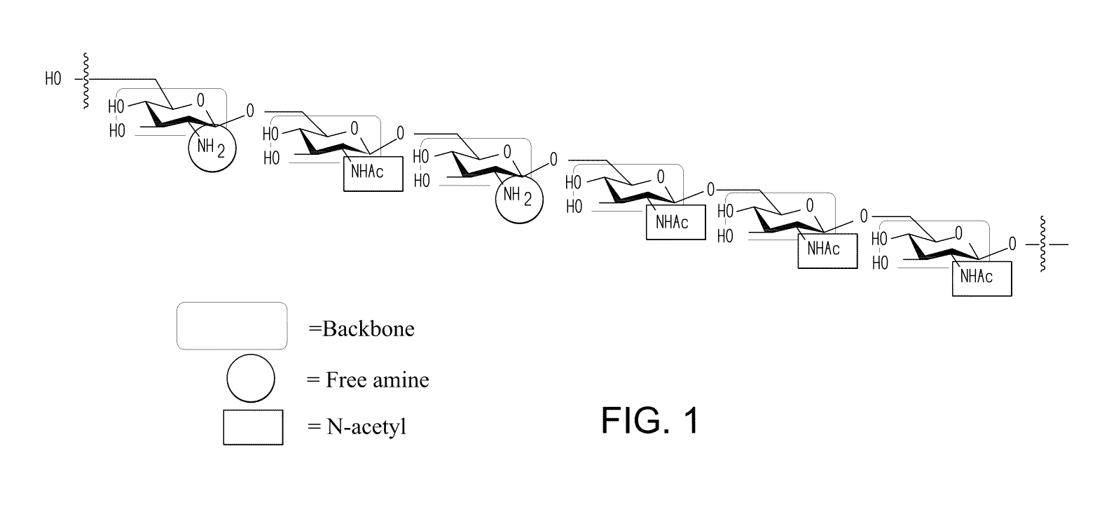

### DETAILED DESCRIPTION

### Definitions

In order to provide a clear and consistent understanding of the specification and claims, the following definitions are provided.

Units, prefixes, and symbols may be denoted in their SI accepted form. Numeric ranges recited herein are inclusive of the numbers defining the range and include and are supportive of each integer within the defined range. Unless otherwise noted, the terms “a” or “an” are to be construed as meaning “at least one of.” The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

As used herein, “oligosaccharide” refers to a compound containing two or more monosaccharide units. Oligosaccharides are considered to have a reducing end and a non-reducing end, whether or not the monosaccharide unit at the reducing end is in fact a reducing sugar. In accordance with accepted nomenclature, oligosaccharides are depicted herein with the non-reducing end on the left and the reducing end on the right. All oligosaccharides described herein are described with the name or abbreviation for the non-reducing monosaccharide (e.g., Gal), preceded by the configuration of the glycosidic bond (α or β), the ring bond, the ring position of the reducing monosaccharide involved in the bond, and then the name or abbreviation of the reducing monosaccharide (e.g., GlcNAc). The linkage between two sugars may be expressed, for example, as 2,3, 2→3, or 2-3. Each monosaccharide is a pyranose or furanose.

As used herein, “monosaccharide” or “monosaccharide unit” refers to a single sugar residue in an oligosaccharide, including derivatives therefrom. Within the context of an oligosaccharide, an individual monomer unit is a monosaccharide which is (or can be) bound through a hydroxyl group to another monosaccharide.

As used herein, “endotoxin-free” refers to an oligosaccharide that does not contain endotoxins or endotoxin components normally present in isolated bacterial carbohydrates and polysaccharides.

As used herein, “synthetic” refers to material which is substantially or essentially free from components, such as endotoxins, glycolipids, unrelated oligosaccharides, etc., which normally accompany a compound when it is isolated. Typically, synthetic compounds are at least about 90% pure, usually at least about 95%, and preferably at least about 99% pure. Purity can be indicated by a number of means well known in the art. Preferably, purity is measured by HPLC. The identity of the synthetic material can be determined by mass spectroscopy and/or NMR spectroscopy.

As used herein the term “linker” refers to either a bond or a moiety which at one end exhibits a grouping able to enter into a covalent bonding with a reactive functional group of the carrier, e.g. an amino, thiol, or carboxyl group, and at the other end a grouping likewise able to enter into a covalent bonding with a hydroxyl group or an amino group of an oligosaccharide according to the present invention. Between the two functional groups of the linker molecule there is a biocompatible bridging molecule of suitable length, e.g. substituted or unsubstituted heteroalkylene, arylalkylene, alkylene, alkenylene, or (oligo)alkylene glycol groups. Linkers preferably include a substituted or unsubstituted (C₁-C₁₀)alkylene group or an substituted or unsubstituted (C₂-C₁₀)alkenylene group.

As used herein, the term “percentage of N-acetylated monosaccharide units in the oligomer” refers to the number of N-acetylated monosaccharide units in the oligosaccharide z, the percentage of N-acetylated monosaccharide units is y, where y is z/(n+1)*100.

As used herein, the term “carrier” refers to a protein, peptide, lipid, polymer, dendrimer, virosome, virus-like particle (VLP), or combination thereof, which is coupled to the oligosaccharide to enhance the immunogenicity of the resulting oligosaccharide-carrier conjugate to a greater degree than the oligosaccharide alone.

As used herein, “protein carrier” refers to a protein, peptide or fragment thereof, which is coupled or conjugated to an oligosaccharide to enhance the immunogenicity of the resulting oligosaccharide-protein carrier conjugate to a greater degree than the oligosaccharide alone. For example, when used as a carrier, the protein carrier may serve as a T-dependent antigen which can activate and recruit T-cells and thereby augment T-cell dependent antibody production.

As used herein, “conjugated” refers to a chemical linkage, either covalent or non-covalent, that proximally associates an oligosaccharide with a carrier so that the oligosaccharide conjugate has increased immunogenicity relative to an unconjugated oligosaccharide.

As used herein, “conjugate” refers to an oligosaccharide chemically coupled to a carrier through a linker and/or a cross-linking agent.

As used herein, “passive immunity” refers to the administration of antibodies to a subject, whereby the antibodies are produced in a different subject (including subjects of the same and different species) such that the antibodies attach to the surface of the bacteria and cause the bacteria to be phagocytosed or killed.

As used herein, “protective immunity” means that a vaccine or immunization schedule that is administered to a animal induces an immune response that prevents, retards the development of, or reduces the severity of a disease that is caused by a pathogen or diminishes or altogether eliminates the symptoms of the disease. Protective immunity may be predicted based on the ability of serum antibody to activate complement-mediated bactericidal activity or confer passive protection against a bacterial infection in a suitable animal challenge model.

As used herein, “immunoprotective composition” refers to a composition formulated to provide protective immunity in a host.

As used herein, “in a sufficient amount to elicit an immune response” or “in an effective amount to stimulate an immune response” (e.g., to epitopes present in a preparation) means that there is a detectable difference between an immune response indicator measured before and after administration of a particular antigen preparation. Immune response indicators include but are not limited to: antibody titer or specificity, as detected by an assay such as enzyme-linked immunoassay (ELISA), bactericidal assay (e.g., to detect serum bactericidal antibodies), flow cytometry, immunoprecipitation, Ouchter-Lowry immunodiffusion; binding detection assays of, for example, spot, Western blot or antigen arrays; cytotoxicity assays, and the like.

As used herein, “antibody” encompasses polyclonal and monoclonal antibody preparations, as well as preparations including hybrid antibodies, altered antibodies, F(ab′)² fragments, F(ab) molecules, Fv fragments, single chain fragment variable displayed on phage (scFv), single domain antibodies, chimeric antibodies, humanized antibodies, and functional fragments thereof which exhibit immunological binding properties of the parent antibody molecule.

As used herein, “monoclonal antibody” refers to an antibody composition having a homogeneous antibody population. The term is not limited by the manner in which it is made. The term encompasses whole immunoglobulin molecules, as well as Fab molecules, F(ab′)₂ fragments, Fv fragments, single chain fragment variable displayed on phage (scFv), and other molecules that exhibit immunological binding properties of the parent monoclonal antibody molecule.

As used herein, “specifically binds to an antibody” or “specifically immunoreactive with”, when referring to an oligosaccharide, protein or peptide, refers to a binding reaction which is based on and/or is probative of the presence of the antigen in a sample which may also include a heterogeneous population of other molecules. Thus, under designated immunoassay conditions, the specified antibody or antibodies bind(s) to a particular antigen or antigens in a sample and does not bind in a significant amount to other molecules present in the sample. Specific binding to an antibody under such conditions may require an antibody or antiserum that is selected for its specificity for a particular antigen or antigens.

As used herein, “antigen” refers to any substance that may be specifically bound by an antibody molecule.

As used herein, “immunogen” and “immunogenic composition” refer to an antigenic composition capable of initiating lymphocyte activation resulting in an antigen-specific immune response.

As used herein the term “poly-N-acetyl glucosamine” or “PNAG” refers to an oligoglucosamine having 100% acetyl substitution.

As used herein the term “deacetylated poly-N-acetyl glucosamine” or “dPNAG” refers to an oligoglucosamine having less than 100% acetyl substitution. The dPNAG may comprise a mixture of dPNAGs with varying degree of acetylation.

As used herein, “epitope” refers to a site on an antigen to which specific B cells and/or T cells respond. The term is also used interchangeably with “antigenic determinant” or “antigenic determinant site.” B cell epitope sites on proteins, oligosaccharides, or other biopolymers may be composed of moieties from different parts of the macromolecule that have been brought together by folding. Epitopes of this kind are referred to as conformational or discontinuous epitopes, since the site is composed of segments the polymer that are discontinuous in the linear sequence but are continuous in the folded conformation(s). Epitopes that are composed of single segments of biopolymers or other molecules are termed continuous or linear epitopes. T cell epitopes are generally restricted to linear peptides. Antibodies that recognize the same epitope can be identified in a simple immunoassay showing the ability of one antibody to block the binding of another antibody to a target antigen.

As used herein, the term “in a first monosaccharide unit R¹ is H, and in a second monosaccharide unit R¹ or R² is C(O)CH₃, said second monosaccharide unit is located three monosaccharide units from the first monosaccharide unit” refers to a substitution pattern as illustrated in the following scheme:

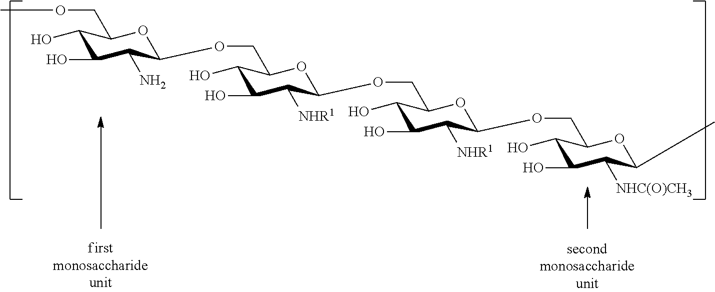

As used herein the term “# sequential C(O)CH₃ groups are located in subsequent monosaccharide units” or “# sequential H groups are located in subsequent monosaccharide units” refers to structure, wherein the respective C(O)CH₃ and H groups are located in adjacent monosaccharide units. The following example illustrates the structural motif for 3 sequential C(O)CH₃ groups:

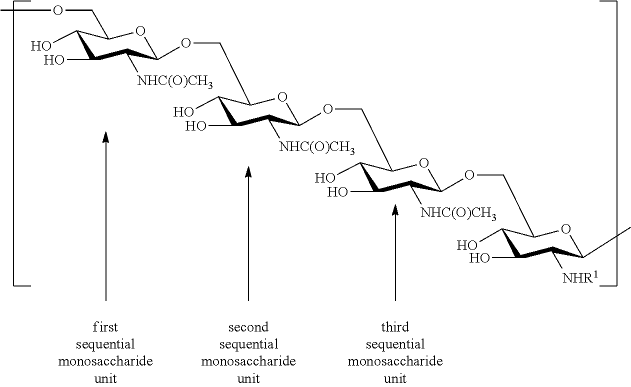

As used herein the term “selective conversion” means that the selectivity of the specific reaction over another reaction is at least 10 fold, at least 100 fold or at least 1000 fold.

The term Ac means acetyl (—C(O)CH₃).

The term TBS means tert-butyldimethylsilyl.

The term Troc means 2,2,2-trichloroethoxycarbonyl.

The term TCl means trichloroacetimidate.

The term Phth means phthaloyl.

The term TFA means trifluoroacetate.

The term TCA means trichloroacetate.

The term Cbz means benzyloxycarbonyl.

The term Bz means benzoyl.

The term Bn means benzyl.

The term TES means triethylsilyl.

The term TBDPS means tert-butyldiphenylsilyl.

The term MCA means monochloracetate.

The term Lev means levulinoyl.

The term ADMB means 4-O-acetyl 12,2 dimethylbutanoate.

The term Tr means triphenylmethyl.

The term DMT means dimethoxytrityl.

The term FMOC means 9-fluorenylmethyl carbonate.

The term Alloc means Allyloxycarbonyl.

The term Nap means napthyl.

The term SEt means thioethyl.

The term SPh means thiophenyl.

The term SToI means thiotolyl.

The term SAdm means thioadamantyl.

Synthetic Oligosaccharides

The present invention provides oligosaccharides 1a:

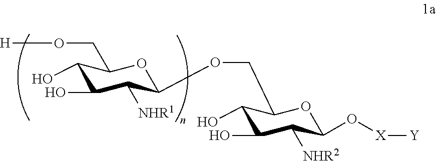

where R¹ and R² are each independently H or COCH₃, where at least one R¹ or R² in the oligosaccharide is H and at least another is COCH₃; n is an integer of at least 3, X is a bond or a linker, and Y is H or a carrier; and where each occurrence of R¹ can be the same or different.

In the oligosaccharide 1a, the number of acetylated monosaccharide units in the oligosaccharide is z and the percentage of acetylated monosaccharide units is y, where y is z/(n+1)*100.

In one embodiment the percentage of the N-acetylated monosaccharide units in the oligomer is 75% or less.

In another embodiment the percentage of the N-acetylated monosaccharide units in the oligomer is 70% or less.

In another embodiment the percentage of the N-acetylated monosaccharide units in the oligomer is 60% or less.

In another embodiment the percentage of the N-acetylated monosaccharide units in the oligomer is 50% or less.

In another embodiment the percentage of the N-acetylated monosaccharide units in the oligomer is 40% or less.

In another embodiment the percentage of the N-acetylated monosaccharide units in the oligomer is 30% or less.

In another embodiment the percentage of the N-acetylated monosaccharide units in the oligomer is 20% or less.

In one embodiment the percentage of the N-acetylated monosaccharide units in the oligomer is at least 15%.

In one embodiment the percentage of the N-acetylated monosaccharide units in the oligomer is at least 20%.

In one embodiment the percentage of the N-acetylated monosaccharide units in the oligomer is at least 30%.

In one embodiment the percentage of the N-acetylated monosaccharide units in the oligomer is at least 40%.

In one embodiment the percentage of the N-acetylated monosaccharide units in the oligomer is at least 45%.

In one embodiment the percentage of the N-acetylated monosaccharide units in the oligomer is at least 50%.

In one embodiment the percentage of the N-acetylated monosaccharide units in the oligomer is at least 55%.

In one embodiment the percentage of the N-acetylated monosaccharide units in the oligomer is at least 60%.

In one embodiment the percentage of the N-acetylated monosaccharide units in the oligomer is at least 65%.

In one embodiment the percentage of the N-acetylated monosaccharide units in the oligomer is between 10% and 70%, 20% to 70%, 30% to 70%, 30% to 60%, or 30% to 50%.

In one embodiment at least 3 of the R¹ and R² groups are C(O)CH₃.

In one embodiment at least 3 of the R¹ and R² groups are H.

In one embodiment n is 5 and 3 of the R¹ or R² groups are C(O)CH₃.

In one embodiment n is 6 or 7 and at least 4 of the R¹ or R² groups are C(O)CH₃.

In one embodiment n is 8 or 9 and at least 5 of the R¹ or R² groups are C(O)CH₃.

In one embodiment n is 10 or 11 and at least 6 of the R¹ or R² groups are C(O)CH₃.

In one embodiment n is 5 and 3 of the R¹ or R² groups are H.

In one embodiment n is 6 or 7 and at least 4 of the R¹ or R² groups are H.

In one embodiment n is 8 or 9 and at least 5 of the R¹ or R² groups are H.

In one embodiment n is 10 or 11 and at least 6 of the R¹ or R² groups are H. In one embodiment the group X is a bond.

In another embodiment the group X is a linker.

In one embodiment the carrier group Y is H.

In another embodiment the carrier group Y is a carrier.

In one embodiment, the X is a bond and Y is —H.

Specific embodiments of the present invention are shown below:

Exemplary embodiments of the present invention are shown in the following Tables 1 to 9.tetrasaccharides (n=3, Table 1), pentasaccharides (n=4, Table 2), hexasaccharides (n=5, Table 3), heptasaccharides (n=6, Table 4), octasaccharides (n=7, Table 5), nonasaccharides (n=8, Table 6), decasaccharides (n=9, Table 7), undecasaccharides (n=10, Table 8), and dodecasaccharides (n=11, Table 9).

In the following tables, the nomenclature “n#, R1″ (i.e., n1′ R1)” identifies the monomeric unit in an oligosaccharide of (n+1) units. The first unit is attached to the monosaccharide bearing R² and the second, third, etc. units follow.

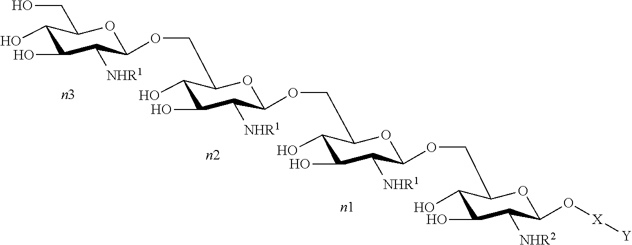

For example, when n is 3, the oligosaccharide (a tetrasaccharide) is shown below:

TABLE 1
Tetrasaccharides (Y is either H or a carrier)

| Compound | R2 | n1, R1 | n2, R1 | n3, R1 |
|---|---|---|---|---|
| 1001 | H | H | H | acetyl |
| 1002 | H | H | Acetyl | H |
| 1003 | H | H | Acetyl | acetyl |
| 1004 | H | acetyl | H | H |
| 1005 | H | acetyl | H | acetyl |
| 1006 | H | acetyl | Acetyl | H |
| 1007 | H | acetyl | acetyl | acetyl |
| 1008 | acetyl | H | H | H |
| 1009 | acetyl | H | H | acetyl |
| 1010 | acetyl | H | acetyl | H |
| 1011 | acetyl | H | acetyl | acetyl |
| 1012 | acetyl | acetyl | H | H |
| 1013 | acetyl | acetyl | H | acetyl |
| 1014 | acetyl | acetyl | acetyl | H |

TABLE 2
(pentasaccharides) (Y is either H or a carrier)

| Compound | R2 | n1, R1 | n2, R1 | n3, R1 | n4, R1 |
|---|---|---|---|---|---|
| 1015 | H | H | H | H | acetyl |
| 1016 | H | H | H | acetyl | H |
| 1017 | H | H | H | acetyl | acetyl |
| 1018 | H | H | acetyl | H | H |
| 1019 | H | H | acetyl | H | acetyl |
| 1020 | H | H | acetyl | acetyl | H |
| 1021 | H | H | acetyl | acetyl | acetyl |
| 1022 | H | acetyl | H | H | H |
| 1023 | H | acetyl | H | H | acetyl |
| 1024 | H | acetyl | H | acetyl | H |
| 1025 | H | acetyl | H | acetyl | acetyl |
| 1026 | H | acetyl | acetyl | H | H |
| 1027 | H | acetyl | acetyl | H | acetyl |
| 1028 | H | acetyl | acetyl | acetyl | H |
| 1029 | H | acetyl | acetyl | acetyl | acetyl |
| 1030 | acetyl | H | H | H | H |
| 1031 | acetyl | H | H | H | acetyl |
| 1032 | acetyl | H | H | acetyl | H |
| 1033 | acetyl | H | H | acetyl | acetyl |
| 1034 | acetyl | H | acetyl | H | H |
| 1035 | acetyl | H | acetyl | H | acetyl |
| 1036 | acetyl | H | acetyl | acetyl | H |
| 1037 | acetyl | H | acetyl | acetyl | acetyl |
| 1038 | acetyl | acetyl | H | H | H |
| 1039 | acetyl | acetyl | H | H | acetyl |
| 1040 | acetyl | acetyl | H | acetyl | H |
| 1041 | acetyl | acetyl | H | acetyl | acetyl |
| 1042 | acetyl | acetyl | acetyl | H | H |
| 1043 | acetyl | acetyl | acetyl | H | acetyl |
| 1044 | acetyl | acetyl | acetyl | acetyl | H |

TABLE 3
(hexasaccharides) (Y is either H or a carrier)

| Compound | R2 | n1, R1 | n2, R1 | n3, R1 | n4, R1 | n5, R1 |
|---|---|---|---|---|---|---|
| 1045 | H | H | H | H | H | acetyl |
| 1046 | H | H | H | H | acetyl | H |
| 1047 | H | H | H | H | acetyl | acetyl |
| 1048 | H | H | H | acetyl | H | H |
| 1049 | H | H | H | acetyl | H | acetyl |
| 1050 | H | H | H | acetyl | acetyl | H |
| 1051 | H | H | H | acetyl | acetyl | acetyl |
| 1052 | H | H | Acetyl | H | H | H |
| 1053 | H | H | Acetyl | H | H | acetyl |
| 1054 | H | H | Acetyl | H | acetyl | H |
| 1055 | H | H | Acetyl | H | acetyl | acetyl |
| 1056 | H | H | Acetyl | acetyl | H | H |
| 1057 | H | H | Acetyl | acetyl | H | acetyl |
| 1058 | H | H | Acetyl | acetyl | acetyl | H |
| 1059 | H | H | Acetyl | acetyl | acetyl | acetyl |
| 1060 | H | acetyl | H | H | H | H |
| 1061 | H | acetyl | H | H | H | acetyl |
| 1062 | H | acetyl | H | H | acetyl | H |
| 1063 | H | acetyl | H | H | acetyl | acetyl |
| 1064 | H | acetyl | H | acetyl | H | H |
| 1065 | H | acetyl | H | acetyl | H | acetyl |
| 1066 | H | acetyl | H | acetyl | acetyl | H |
| 1067 | H | acetyl | H | acetyl | acetyl | acetyl |
| 1068 | H | acetyl | Acetyl | H | H | H |
| 1069 | H | acetyl | Acetyl | H | H | acetyl |
| 1070 | H | acetyl | Acetyl | H | acetyl | H |
| 1071 | H | acetyl | Acetyl | H | acetyl | acetyl |
| 1072 | H | acetyl | Acetyl | acetyl | H | H |
| 1073 | H | acetyl | Acetyl | acetyl | H | acetyl |
| 1074 | H | acetyl | Acetyl | acetyl | acetyl | H |
| 1075 | H | acetyl | Acetyl | acetyl | acetyl | acetyl |
| 1076 | acetyl | H | H | H | H | H |
| 1077 | acetyl | H | H | H | H | acetyl |
| 1078 | acetyl | H | H | H | acetyl | H |
| 1079 | acetyl | H | H | H | acetyl | acetyl |
| 1080 | acetyl | H | H | acetyl | H | H |
| 1081 | acetyl | H | H | acetyl | H | acetyl |
| 1082 | acetyl | H | H | acetyl | acetyl | H |
| 1083 | acetyl | H | H | acetyl | acetyl | acetyl |
| 1084 | acetyl | H | Acetyl | H | H | H |
| 1085 | acetyl | H | Acetyl | H | H | acetyl |
| 1086 | acetyl | H | Acetyl | H | acetyl | H |
| 1087 | acetyl | H | Acetyl | H | acetyl | acetyl |
| 1088 | acetyl | H | Acetyl | acetyl | H | H |
| 1089 | acetyl | H | Acetyl | acetyl | H | acetyl |
| 1090 | acetyl | H | Acetyl | acetyl | acetyl | H |
| 1091 | acetyl | H | Acetyl | acetyl | acetyl | acetyl |
| 1092 | acetyl | acetyl | H | H | H | H |
| 1093 | acetyl | acetyl | H | H | H | acetyl |
| 1094 | acetyl | acetyl | H | H | acetyl | H |
| 1095 | acetyl | acetyl | H | H | acetyl | acetyl |
| 1096 | acetyl | acetyl | H | acetyl | H | H |
| 1097 | acetyl | acetyl | H | acetyl | H | acetyl |
| 1098 | acetyl | acetyl | H | acetyl | acetyl | H |
| 1099 | acetyl | acetyl | H | acetyl | acetyl | acetyl |
| 1100 | acetyl | acetyl | Acetyl | H | H | H |
| 1101 | acetyl | acetyl | Acetyl | H | H | acetyl |
| 1102 | acetyl | acetyl | Acetyl | H | acetyl | H |
| 1103 | acetyl | acetyl | Acetyl | H | acetyl | acetyl |
| 1104 | acetyl | acetyl | Acetyl | acetyl | H | H |
| 1105 | acetyl | acetyl | Acetyl | acetyl | H | acetyl |
| 1106 | acetyl | acetyl | Acetyl | acetyl | acetyl | H |

TABLE 4
(heptasaccharides) (Y is either H or a carrier)

| | | | n2, | | n4, | | |
|---|---|---|---|---|---|---|---|
| Compound | R2 | n1, R1 | R1 | n3, R1 | R1 | n5, R1 | n6, R1 |
| 1107 | H | H | H | H | H | H | acetyl |
| 1108 | H | H | H | H | H | acetyl | H |
| 1109 | H | H | H | H | H | acetyl | acetyl |
| 1110 | H | H | H | H | acetyl | H | H |
| 1111 | H | H | H | H | acetyl | H | acetyl |
| 1112 | H | H | H | H | acetyl | acetyl | H |
| 1113 | H | H | H | H | acetyl | acetyl | acetyl |
| 1114 | H | H | H | acetyl | H | H | H |
| 1115 | H | H | H | acetyl | H | H | acetyl |
| 1116 | H | H | H | acetyl | H | acetyl | H |
| 1117 | H | H | H | acetyl | H | acetyl | acetyl |
| 1118 | H | H | H | acetyl | acetyl | H | H |
| 1119 | H | H | H | acetyl | acetyl | H | acetyl |
| 1120 | H | H | H | acetyl | acetyl | acetyl | H |
| 1121 | H | H | H | acetyl | acetyl | acetyl | acetyl |
| 1122 | H | H | acetyl | H | H | H | H |
| 1123 | H | H | acetyl | H | H | H | acetyl |
| 1124 | H | H | acetyl | H | H | acetyl | H |
| 1125 | H | H | acetyl | H | H | acetyl | acetyl |
| 1126 | H | H | acetyl | H | acetyl | H | H |
| 1127 | H | H | acetyl | H | acetyl | H | acetyl |
| 1128 | H | H | acetyl | H | acetyl | acetyl | H |
| 1129 | H | H | acetyl | H | acetyl | acetyl | acetyl |
| 1130 | H | H | acetyl | acetyl | H | H | H |
| 1131 | H | H | acetyl | acetyl | H | H | acetyl |
| 1132 | H | H | acetyl | acetyl | H | acetyl | H |
| 1133 | H | H | acetyl | acetyl | H | acetyl | acetyl |
| 1134 | H | H | acetyl | acetyl | acetyl | H | H |
| 1135 | H | H | acetyl | acetyl | acetyl | H | acetyl |
| 1136 | H | H | acetyl | acetyl | acetyl | acetyl | H |
| 1137 | H | H | acetyl | acetyl | acetyl | acetyl | acetyl |
| 1138 | H | acetyl | H | H | H | H | H |
| 1139 | H | acetyl | H | H | H | H | acetyl |
| 1140 | H | acetyl | H | H | H | acetyl | H |
| 1141 | H | acetyl | H | H | H | acetyl | acetyl |
| 1142 | H | acetyl | H | H | acetyl | H | H |
| 1143 | H | acetyl | H | H | acetyl | H | acetyl |
| 1144 | H | acetyl | H | H | acetyl | acetyl | H |
| 1145 | H | acetyl | H | H | acetyl | acetyl | acetyl |
| 1146 | H | acetyl | H | acetyl | H | H | H |
| 1147 | H | acetyl | H | acetyl | H | H | acetyl |
| 1148 | H | acetyl | H | acetyl | H | acetyl | H |
| 1149 | H | acetyl | H | acetyl | H | acetyl | acetyl |
| 1150 | H | acetyl | H | acetyl | acetyl | H | H |
| 1151 | H | acetyl | H | acetyl | acetyl | H | acetyl |
| 1152 | H | acetyl | H | acetyl | acetyl | acetyl | H |
| 1153 | H | acetyl | H | acetyl | acetyl | acetyl | acetyl |
| 1154 | H | acetyl | acetyl | H | H | H | H |
| 1155 | H | acetyl | acetyl | H | H | H | acetyl |
| 1156 | H | acetyl | acetyl | H | H | acetyl | H |
| 1157 | H | acetyl | acetyl | H | H | acetyl | acetyl |
| 1158 | H | acetyl | acetyl | H | acetyl | H | H |
| 1159 | H | acetyl | acetyl | H | acetyl | H | acetyl |
| 1160 | H | acetyl | acetyl | H | acetyl | acetyl | H |
| 1161 | H | acetyl | acetyl | H | acetyl | acetyl | acetyl |
| 1162 | H | acetyl | acetyl | acetyl | H | H | H |
| 1163 | H | acetyl | acetyl | acetyl | H | H | acetyl |
| 1164 | H | acetyl | acetyl | acetyl | H | acetyl | H |
| 1165 | H | acetyl | acetyl | acetyl | H | acetyl | acetyl |
| 1166 | H | acetyl | acetyl | acetyl | acetyl | H | H |
| 1167 | H | acetyl | acetyl | acetyl | acetyl | H | acetyl |
| 1168 | H | acetyl | acetyl | acetyl | acetyl | acetyl | H |
| 1169 | H | acetyl | acetyl | acetyl | acetyl | acetyl | acetyl |
| 1170 | acetyl | H | H | H | H | H | H |
| 1171 | acetyl | H | H | H | H | H | acetyl |
| 1172 | acetyl | H | H | H | H | acetyl | H |
| 1173 | acetyl | H | H | H | H | acetyl | acetyl |
| 1174 | acetyl | H | H | H | acetyl | H | H |
| 1175 | acetyl | H | H | H | acetyl | H | acetyl |
| 1176 | acetyl | H | H | H | acetyl | acetyl | H |
| 1177 | acetyl | H | H | H | acetyl | acetyl | acetyl |
| 1178 | acetyl | H | H | acetyl | H | H | H |
| 1179 | acetyl | H | H | acetyl | H | H | acetyl |
| 1180 | acetyl | H | H | acetyl | H | acetyl | H |
| 1181 | acetyl | H | H | acetyl | H | acetyl | acetyl |
| 1182 | acetyl | H | H | acetyl | acetyl | H | H |
| 1183 | acetyl | H | H | acetyl | acetyl | H | acetyl |
| 1184 | acetyl | H | H | acetyl | acetyl | acetyl | H |
| 1185 | acetyl | H | H | acetyl | acetyl | acetyl | acetyl |
| 1186 | acetyl | H | acetyl | H | H | H | H |
| 1187 | acetyl | H | acetyl | H | H | H | acetyl |
| 1188 | acetyl | H | acetyl | H | H | acetyl | H |
| 1189 | acetyl | H | acetyl | H | H | acetyl | acetyl |
| 1190 | acetyl | H | acetyl | H | acetyl | H | H |
| 1191 | acetyl | H | acetyl | H | acetyl | H | acetyl |
| 1192 | acetyl | H | acetyl | H | acetyl | acetyl | H |
| 1193 | acetyl | H | acetyl | H | acetyl | acetyl | acetyl |
| 1194 | acetyl | H | acetyl | acetyl | H | H | H |
| 1195 | acetyl | H | acetyl | acetyl | H | H | acetyl |
| 1196 | acetyl | H | acetyl | acetyl | H | acetyl | H |
| 1197 | acetyl | H | acetyl | acetyl | H | acetyl | acetyl |
| 1198 | acetyl | H | acetyl | acetyl | acetyl | H | H |
| 1199 | acetyl | H | acetyl | acetyl | acetyl | H | acetyl |
| 1200 | acetyl | H | acetyl | acetyl | acetyl | acetyl | H |
| 1201 | acetyl | H | acetyl | acetyl | acetyl | acetyl | acetyl |
| 1202 | acetyl | acetyl | H | H | H | H | H |
| 1203 | acetyl | acetyl | H | H | H | H | acetyl |
| 1204 | acetyl | acetyl | H | H | H | acetyl | H |
| 1205 | acetyl | acetyl | H | H | H | acetyl | acetyl |
| 1206 | acetyl | acetyl | H | H | acetyl | H | H |
| 1207 | acetyl | acetyl | H | H | acetyl | H | acetyl |
| 1208 | acetyl | acetyl | H | H | acetyl | acetyl | H |
| 1209 | acetyl | acetyl | H | H | acetyl | acetyl | acetyl |
| 1210 | acetyl | acetyl | H | acetyl | H | H | H |
| 1211 | acetyl | acetyl | H | acetyl | H | H | acetyl |
| 1212 | acetyl | acetyl | H | acetyl | H | acetyl | H |
| 1213 | acetyl | acetyl | H | acetyl | H | acetyl | acetyl |
| 1214 | acetyl | acetyl | H | acetyl | acetyl | H | H |
| 1215 | acetyl | acetyl | H | acetyl | acetyl | H | acetyl |
| 1216 | acetyl | acetyl | H | acetyl | acetyl | acetyl | H |
| 1217 | acetyl | acetyl | H | acetyl | acetyl | acetyl | acetyl |
| 1218 | acetyl | acetyl | acetyl | H | H | H | H |
| 1219 | acetyl | acetyl | acetyl | H | H | H | acetyl |
| 1220 | acetyl | acetyl | acetyl | H | H | acetyl | H |
| 1221 | acetyl | acetyl | acetyl | H | H | acetyl | acetyl |
| 1222 | acetyl | acetyl | acetyl | H | acetyl | H | H |
| 1223 | acetyl | acetyl | acetyl | H | acetyl | H | acetyl |
| 1224 | acetyl | acetyl | acetyl | H | acetyl | acetyl | H |
| 1225 | acetyl | acetyl | acetyl | H | acetyl | acetyl | acetyl |
| 1226 | acetyl | acetyl | acetyl | acetyl | H | H | H |
| 1227 | acetyl | acetyl | acetyl | acetyl | H | H | acetyl |
| 1228 | acetyl | acetyl | acetyl | acetyl | H | acetyl | H |
| 1229 | acetyl | acetyl | acetyl | acetyl | H | acetyl | acetyl |
| 1230 | acetyl | acetyl | acetyl | acetyl | acetyl | H | H |
| 1231 | acetyl | acetyl | acetyl | acetyl | acetyl | H | acetyl |
| 1232 | acetyl | acetyl | acetyl | acetyl | acetyl | acetyl | H |

TABLE 5
(octasaccharides) (Y is either H or a carrier)

| | | | n2, | n3, | n4, | n5, | n6, | n7, |
|---|---|---|---|---|---|---|---|---|
| Compound | R2 | n1, R1 | R1 | R1 | R1 | R1 | R1 | R1 |
| 1233 | H | H | H | H | H | H | H | acetyl |
| 1234 | H | H | H | H | H | H | acetyl | H |
| 1235 | H | H | H | H | H | H | acetyl | acetyl |
| 1236 | H | H | H | H | H | acetyl | H | H |
| 1237 | H | H | H | H | H | acetyl | H | acetyl |
| 1238 | H | H | H | H | H | acetyl | acetyl | H |
| 1239 | H | H | H | H | H | acetyl | acetyl | acetyl |
| 1240 | H | H | H | H | acetyl | H | H | H |
| 1241 | H | H | H | H | acetyl | H | H | acetyl |
| 1242 | H | H | H | H | acetyl | H | acetyl | H |
| 1243 | H | H | H | H | acetyl | H | acetyl | acetyl |
| 1244 | H | H | H | H | acetyl | acetyl | H | H |
| 1245 | H | H | H | H | acetyl | acetyl | H | acetyl |
| 1246 | H | H | H | H | acetyl | acetyl | acetyl | H |
| 1247 | H | H | H | H | acetyl | acetyl | acetyl | acetyl |
| 1248 | H | H | H | acetyl | H | H | H | H |
| 1249 | H | H | H | acetyl | H | H | H | acetyl |
| 1250 | H | H | H | acetyl | H | H | acetyl | H |
| 1251 | H | H | H | acetyl | H | H | acetyl | acetyl |
| 1252 | H | H | H | acetyl | H | acetyl | H | H |
| 1253 | H | H | H | acetyl | H | acetyl | H | acetyl |
| 1254 | H | H | H | acetyl | H | acetyl | acetyl | H |
| 1255 | H | H | H | acetyl | H | acetyl | acetyl | acetyl |
| 1256 | H | H | H | acetyl | acetyl | H | H | H |
| 1257 | H | H | H | acetyl | acetyl | H | H | acetyl |
| 1258 | H | H | H | acetyl | acetyl | H | acetyl | H |
| 1259 | H | H | H | acetyl | acetyl | H | acetyl | acetyl |
| 1260 | H | H | H | acetyl | acetyl | acetyl | H | H |
| 1261 | H | H | H | acetyl | acetyl | acetyl | H | acetyl |
| 1262 | H | H | H | acetyl | acetyl | acetyl | acetyl | H |
| 1263 | H | H | H | acetyl | acetyl | acetyl | acetyl | acetyl |
| 1264 | H | H | acetyl | H | H | H | H | H |
| 1265 | H | H | acetyl | H | H | H | H | acetyl |
| 1266 | H | H | acetyl | H | H | H | acetyl | H |
| 1267 | H | H | acetyl | H | H | H | acetyl | acetyl |
| 1268 | H | H | acetyl | H | H | acetyl | H | H |
| 1269 | H | H | acetyl | H | H | acetyl | H | acetyl |
| 1270 | H | H | acetyl | H | H | acetyl | acetyl | H |
| 1271 | H | H | acetyl | H | H | acetyl | acetyl | acetyl |
| 1272 | H | H | acetyl | H | acetyl | H | H | H |
| 1273 | H | H | acetyl | H | acetyl | H | H | acetyl |
| 1274 | H | H | acetyl | H | acetyl | H | acetyl | H |
| 1275 | H | H | acetyl | H | acetyl | H | acetyl | acetyl |
| 1276 | H | H | acetyl | H | acetyl | acetyl | H | H |
| 1277 | H | H | acetyl | H | acetyl | acetyl | H | acetyl |
| 1278 | H | H | acetyl | H | acetyl | acetyl | acetyl | H |
| 1279 | H | H | acetyl | H | acetyl | acetyl | acetyl | acetyl |
| 1280 | H | H | acetyl | acetyl | H | H | H | H |
| 1281 | H | H | acetyl | acetyl | H | H | H | acetyl |
| 1282 | H | H | acetyl | acetyl | H | H | acetyl | H |
| 1283 | H | H | acetyl | acetyl | H | H | acetyl | acetyl |
| 1284 | H | H | acetyl | acetyl | H | acetyl | H | H |
| 1285 | H | H | acetyl | acetyl | H | acetyl | H | acetyl |
| 1286 | H | H | acetyl | acetyl | H | acetyl | acetyl | H |
| 1287 | H | H | acetyl | acetyl | H | acetyl | acetyl | acetyl |
| 1288 | H | H | acetyl | acetyl | acetyl | H | H | H |
| 1289 | H | H | acetyl | acetyl | acetyl | H | H | acetyl |
| 1290 | H | H | acetyl | acetyl | acetyl | H | acetyl | H |
| 1291 | H | H | acetyl | acetyl | acetyl | H | acetyl | acetyl |
| 1292 | H | H | acetyl | acetyl | acetyl | acetyl | H | H |
| 1293 | H | H | acetyl | acetyl | acetyl | acetyl | H | acetyl |
| 1294 | H | H | acetyl | acetyl | acetyl | acetyl | acetyl | H |
| 1295 | H | H | acetyl | acetyl | acetyl | acetyl | acetyl | acetyl |
| 1296 | H | acetyl | H | H | H | H | H | H |
| 1297 | H | acetyl | H | H | H | H | H | acetyl |
| 1298 | H | acetyl | H | H | H | H | acetyl | H |
| 1299 | H | acetyl | H | H | H | H | acetyl | acetyl |
| 1300 | H | acetyl | H | H | H | acetyl | H | H |
| 1301 | H | acetyl | H | H | H | acetyl | H | acetyl |
| 1302 | H | acetyl | H | H | H | acetyl | acetyl | H |
| 1303 | H | acetyl | H | H | H | acetyl | acetyl | acetyl |
| 1304 | H | acetyl | H | H | acetyl | H | H | H |
| 1305 | H | acetyl | H | H | acetyl | H | H | acetyl |
| 1306 | H | acetyl | H | H | acetyl | H | acetyl | H |
| 1307 | H | acetyl | H | H | acetyl | H | acetyl | acetyl |
| 1308 | H | acetyl | H | H | acetyl | acetyl | H | H |
| 1309 | H | acetyl | H | H | acetyl | acetyl | H | acetyl |
| 1310 | H | acetyl | H | H | acetyl | acetyl | acetyl | H |
| 1311 | H | acetyl | H | H | acetyl | acetyl | acetyl | acetyl |
| 1312 | H | acetyl | H | acetyl | H | H | H | H |
| 1313 | H | acetyl | H | acetyl | H | H | H | acetyl |
| 1314 | H | acetyl | H | acetyl | H | H | acetyl | H |
| 1315 | H | acetyl | H | acetyl | H | H | acetyl | acetyl |
| 1316 | H | acetyl | H | acetyl | H | acetyl | H | H |
| 1317 | H | acetyl | H | acetyl | H | acetyl | H | acetyl |
| 1318 | H | acetyl | H | acetyl | H | acetyl | acetyl | H |
| 1319 | H | acetyl | H | acetyl | H | acetyl | acetyl | acetyl |
| 1320 | H | acetyl | H | acetyl | acetyl | H | H | H |
| 1321 | H | acetyl | H | acetyl | acetyl | H | H | acetyl |
| 1322 | H | acetyl | H | acetyl | acetyl | H | acetyl | H |
| 1323 | H | acetyl | H | acetyl | acetyl | H | acetyl | acetyl |
| 1324 | H | acetyl | H | acetyl | acetyl | acetyl | H | H |
| 1325 | H | acetyl | H | acetyl | acetyl | acetyl | H | acetyl |
| 1326 | H | acetyl | H | acetyl | acetyl | acetyl | acetyl | H |
| 1327 | H | acetyl | H | acetyl | acetyl | acetyl | acetyl | acetyl |
| 1328 | H | acetyl | acetyl | H | H | H | H | H |
| 1329 | H | acetyl | acetyl | H | H | H | H | acetyl |
| 1330 | H | acetyl | acetyl | H | H | H | acetyl | H |
| 1331 | H | acetyl | acetyl | H | H | H | acetyl | acetyl |
| 1332 | H | acetyl | acetyl | H | H | acetyl | H | H |
| 1333 | H | acetyl | acetyl | H | H | acetyl | H | acetyl |
| 1334 | H | acetyl | acetyl | H | H | acetyl | acetyl | H |
| 1335 | H | acetyl | acetyl | H | H | acetyl | acetyl | acetyl |
| 1336 | H | acetyl | acetyl | H | acetyl | H | H | H |
| 1337 | H | acetyl | acetyl | H | acetyl | H | H | acetyl |
| 1338 | H | acetyl | acetyl | H | acetyl | H | acetyl | H |
| 1339 | H | acetyl | acetyl | H | acetyl | H | acetyl | acetyl |
| 1340 | H | acetyl | acetyl | H | acetyl | acetyl | H | H |
| 1341 | H | acetyl | acetyl | H | acetyl | acetyl | H | acetyl |
| 1342 | H | acetyl | acetyl | H | acetyl | acetyl | acetyl | H |
| 1343 | H | acetyl | acetyl | H | acetyl | acetyl | acetyl | acetyl |
| 1344 | H | acetyl | acetyl | acetyl | H | H | H | H |
| 1345 | H | acetyl | acetyl | acetyl | H | H | H | acetyl |
| 1346 | H | acetyl | acetyl | acetyl | H | H | acetyl | H |
| 1347 | H | acetyl | acetyl | acetyl | H | H | acetyl | acetyl |
| 1348 | H | acetyl | acetyl | acetyl | H | acetyl | H | H |
| 1349 | H | acetyl | acetyl | acetyl | H | acetyl | H | acetyl |
| 1350 | H | acetyl | acetyl | acetyl | H | acetyl | acetyl | H |
| 1351 | H | acetyl | acetyl | acetyl | H | acetyl | acetyl | acetyl |
| 1352 | H | acetyl | acetyl | acetyl | acetyl | H | H | H |
| 1353 | H | acetyl | acetyl | acetyl | acetyl | H | H | acetyl |
| 1354 | H | acetyl | acetyl | acetyl | acetyl | H | acetyl | H |
| 1355 | H | acetyl | acetyl | acetyl | acetyl | H | acetyl | acetyl |
| 1356 | H | acetyl | acetyl | acetyl | acetyl | acetyl | H | H |
| 1357 | H | acetyl | acetyl | acetyl | acetyl | acetyl | H | acetyl |
| 1358 | H | acetyl | acetyl | acetyl | acetyl | acetyl | acetyl | H |
| 1359 | H | acetyl | acetyl | acetyl | acetyl | acetyl | acetyl | acetyl |
| 1360 | acetyl | H | H | H | H | H | H | H |
| 1361 | acetyl | H | H | H | H | H | H | acetyl |
| 1362 | acetyl | H | H | H | H | H | acetyl | H |
| 1363 | acetyl | H | H | H | H | H | acetyl | acetyl |
| 1364 | acetyl | H | H | H | H | acetyl | H | H |
| 1365 | acetyl | H | H | H | H | acetyl | H | acetyl |
| 1366 | acetyl | H | H | H | H | acetyl | acetyl | H |
| 1367 | acetyl | H | H | H | H | acetyl | acetyl | acetyl |
| 1368 | acetyl | H | H | H | acetyl | H | H | H |
| 1369 | acetyl | H | H | H | acetyl | H | H | acetyl |
| 1370 | acetyl | H | H | H | acetyl | H | acetyl | H |
| 1371 | acetyl | H | H | H | acetyl | H | acetyl | acetyl |
| 1372 | acetyl | H | H | H | acetyl | acetyl | H | H |
| 1373 | acetyl | H | H | H | acetyl | acetyl | H | acetyl |
| 1374 | acetyl | H | H | H | acetyl | acetyl | acetyl | H |
| 1375 | acetyl | H | H | H | acetyl | acetyl | acetyl | acetyl |
| 1376 | acetyl | H | H | acetyl | H | H | H | H |
| 1377 | acetyl | H | H | acetyl | H | H | H | acetyl |
| 1378 | acetyl | H | H | acetyl | H | H | acetyl | H |
| 1379 | acetyl | H | H | acetyl | H | H | acetyl | acetyl |
| 1380 | acetyl | H | H | acetyl | H | acetyl | H | H |
| 1381 | acetyl | H | H | acetyl | H | acetyl | H | acetyl |
| 1382 | acetyl | H | H | acetyl | H | acetyl | acetyl | H |
| 1383 | acetyl | H | H | acetyl | H | acetyl | acetyl | acetyl |
| 1384 | acetyl | H | H | acetyl | acetyl | H | H | H |
| 1385 | acetyl | H | H | acetyl | acetyl | H | H | acetyl |
| 1386 | acetyl | H | H | acetyl | acetyl | H | acetyl | H |
| 1387 | acetyl | H | H | acetyl | acetyl | H | acetyl | acetyl |
| 1388 | acetyl | H | H | acetyl | acetyl | acetyl | H | H |
| 1389 | acetyl | H | H | acetyl | acetyl | acetyl | H | acetyl |
| 1390 | acetyl | H | H | acetyl | acetyl | acetyl | acetyl | H |
| 1391 | acetyl | H | H | acetyl | acetyl | acetyl | acetyl | acetyl |
| 1392 | acetyl | H | acetyl | H | H | H | H | H |
| 1393 | acetyl | H | acetyl | H | H | H | H | acetyl |
| 1394 | acetyl | H | acetyl | H | H | H | acetyl | H |
| 1395 | acetyl | H | acetyl | H | H | H | acetyl | acetyl |
| 1396 | acetyl | H | acetyl | H | H | acetyl | H | H |
| 1397 | acetyl | H | acetyl | H | H | acetyl | H | acetyl |
| 1398 | acetyl | H | acetyl | H | H | acetyl | acetyl | H |
| 1399 | acetyl | H | acetyl | H | H | acetyl | acetyl | acetyl |
| 1400 | acetyl | H | acetyl | H | acetyl | H | H | H |
| 1401 | acetyl | H | acetyl | H | acetyl | H | H | acetyl |
| 1402 | acetyl | H | acetyl | H | acetyl | H | acetyl | H |
| 1403 | acetyl | H | acetyl | H | acetyl | H | acetyl | acetyl |
| 1404 | acetyl | H | acetyl | H | acetyl | acetyl | H | H |
| 1405 | acetyl | H | acetyl | H | acetyl | acetyl | H | acetyl |
| 1406 | acetyl | H | acetyl | H | acetyl | acetyl | acetyl | H |
| 1407 | acetyl | H | acetyl | H | acetyl | acetyl | acetyl | acetyl |
| 1408 | acetyl | H | acetyl | acetyl | H | H | H | H |
| 1409 | acetyl | H | acetyl | acetyl | H | H | H | acetyl |
| 1410 | acetyl | H | acetyl | acetyl | H | H | acetyl | H |
| 1411 | acetyl | H | acetyl | acetyl | H | H | acetyl | acetyl |
| 1412 | acetyl | H | acetyl | acetyl | H | acetyl | H | H |
| 1413 | acetyl | H | acetyl | acetyl | H | acetyl | H | acetyl |
| 1414 | acetyl | H | acetyl | acetyl | H | acetyl | acetyl | H |
| 1415 | acetyl | H | acetyl | acetyl | H | acetyl | acetyl | acetyl |
| 1416 | acetyl | H | acetyl | acetyl | acetyl | H | H | H |
| 1417 | acetyl | H | acetyl | acetyl | acetyl | H | H | acetyl |
| 1418 | acetyl | H | acetyl | acetyl | acetyl | H | acetyl | H |
| 1419 | acetyl | H | acetyl | acetyl | acetyl | H | acetyl | acetyl |
| 1420 | acetyl | H | acetyl | acetyl | acetyl | acetyl | H | H |
| 1421 | acetyl | H | acetyl | acetyl | acetyl | acetyl | H | acetyl |
| 1422 | acetyl | H | acetyl | acetyl | acetyl | acetyl | acetyl | H |
| 1423 | acetyl | H | acetyl | acetyl | acetyl | acetyl | acetyl | acetyl |
| 1424 | acetyl | acetyl | H | H | H | H | H | H |
| 1425 | acetyl | acetyl | H | H | H | H | H | acetyl |
| 1426 | acetyl | acetyl | H | H | H | H | acetyl | H |
| 1427 | acetyl | acetyl | H | H | H | H | acetyl | acetyl |
| 1428 | acetyl | acetyl | H | H | H | acetyl | H | H |
| 1429 | acetyl | acetyl | H | H | H | acetyl | H | acetyl |
| 1430 | acetyl | acetyl | H | H | H | acetyl | acetyl | H |
| 1431 | acetyl | acetyl | H | H | H | acetyl | acetyl | acetyl |
| 1432 | acetyl | acetyl | H | H | acetyl | H | H | H |
| 1433 | acetyl | acetyl | H | H | acetyl | H | H | acetyl |
| 1434 | acetyl | acetyl | H | H | acetyl | H | acetyl | H |
| 1435 | acetyl | acetyl | H | H | acetyl | H | acetyl | acetyl |
| 1436 | acetyl | acetyl | H | H | acetyl | acetyl | H | H |
| 1437 | acetyl | acetyl | H | H | acetyl | acetyl | H | acetyl |
| 1438 | acetyl | acetyl | H | H | acetyl | acetyl | acetyl | H |
| 1439 | acetyl | acetyl | H | H | acetyl | acetyl | acetyl | acetyl |
| 1440 | acetyl | acetyl | H | acetyl | H | H | H | H |
| 1441 | acetyl | acetyl | H | acetyl | H | H | H | acetyl |
| 1442 | acetyl | acetyl | H | acetyl | H | H | acetyl | H |
| 1443 | acetyl | acetyl | H | acetyl | H | H | acetyl | acetyl |
| 1444 | acetyl | acetyl | H | acetyl | H | acetyl | H | H |
| 1445 | acetyl | acetyl | H | acetyl | H | acetyl | H | acetyl |
| 1446 | acetyl | acetyl | H | acetyl | H | acetyl | acetyl | H |
| 1447 | acetyl | acetyl | H | acetyl | H | acetyl | acetyl | acetyl |
| 1448 | acetyl | acetyl | H | acetyl | acetyl | H | H | H |
| 1449 | acetyl | acetyl | H | acetyl | acetyl | H | H | acetyl |
| 1450 | acetyl | acetyl | H | acetyl | acetyl | H | acetyl | H |
| 1451 | acetyl | acetyl | H | acetyl | acetyl | H | acetyl | acetyl |
| 1452 | acetyl | acetyl | H | acetyl | acetyl | acetyl | H | H |
| 1453 | acetyl | acetyl | H | acetyl | acetyl | acetyl | H | acetyl |
| 1454 | acetyl | acetyl | H | acetyl | acetyl | acetyl | acetyl | H |
| 1455 | acetyl | acetyl | H | acetyl | acetyl | acetyl | acetyl | acetyl |
| 1456 | acetyl | acetyl | acetyl | H | H | H | H | H |
| 1457 | acetyl | acetyl | acetyl | H | H | H | H | acetyl |
| 1458 | acetyl | acetyl | acetyl | H | H | H | acetyl | H |
| 1459 | acetyl | acetyl | acetyl | H | H | H | acetyl | acetyl |
| 1460 | acetyl | acetyl | acetyl | H | H | acetyl | H | H |
| 1461 | acetyl | acetyl | acetyl | H | H | acetyl | H | acetyl |
| 1462 | acetyl | acetyl | acetyl | H | H | acetyl | acetyl | H |
| 1463 | acetyl | acetyl | acetyl | H | H | acetyl | acetyl | acetyl |
| 1464 | acetyl | acetyl | acetyl | H | acetyl | H | H | H |
| 1465 | acetyl | acetyl | acetyl | H | acetyl | H | H | acetyl |
| 1466 | acetyl | acetyl | acetyl | H | acetyl | H | acetyl | H |
| 1467 | acetyl | acetyl | acetyl | H | acetyl | H | acetyl | acetyl |
| 1468 | acetyl | acetyl | acetyl | H | acetyl | acetyl | H | H |
| 1469 | acetyl | acetyl | acetyl | H | acetyl | acetyl | H | acetyl |
| 1470 | acetyl | acetyl | acetyl | H | acetyl | acetyl | acetyl | H |
| 1471 | acetyl | acetyl | acetyl | H | acetyl | acetyl | acetyl | acetyl |
| 1472 | acetyl | acetyl | acetyl | acetyl | H | H | H | H |
| 1473 | acetyl | acetyl | acetyl | acetyl | H | H | H | acetyl |
| 1474 | acetyl | acetyl | acetyl | acetyl | H | H | acetyl | H |
| 1475 | acetyl | acetyl | acetyl | acetyl | H | H | acetyl | acetyl |
| 1476 | acetyl | acetyl | acetyl | acetyl | H | acetyl | H | H |
| 1477 | acetyl | acetyl | acetyl | acetyl | H | acetyl | H | acetyl |
| 1478 | acetyl | acetyl | acetyl | acetyl | H | acetyl | acetyl | H |
| 1479 | acetyl | acetyl | acetyl | acetyl | H | acetyl | acetyl | acetyl |
| 1480 | acetyl | acetyl | acetyl | acetyl | acetyl | H | H | H |
| 1481 | acetyl | acetyl | acetyl | acetyl | acetyl | H | H | acetyl |
| 1482 | acetyl | acetyl | acetyl | acetyl | acetyl | H | acetyl | H |
| 1483 | acetyl | acetyl | acetyl | acetyl | acetyl | H | acetyl | acetyl |
| 1484 | acetyl | acetyl | acetyl | acetyl | acetyl | acetyl | H | H |
| 1485 | acetyl | acetyl | acetyl | acetyl | acetyl | acetyl | H | acetyl |
| 1486 | acetyl | acetyl | acetyl | acetyl | acetyl | acetyl | acetyl | H |

TABLE 6
(nonasaccharides) (Y is either H or a carrier)

| | | n1, | n2, | n3, | n4, | n5, | n6, |
|---|---|---|---|---|---|---|---|
| Compound | R2 | R1 | R1 | R1 | R1 | R1 | R1 |
| 1107 | H | H | H | H | H | H | acetyl |
| 1108 | H | H | H | H | H | acetyl | H |
| 1109 | H | H | H | H | H | acetyl | acetyl |
| 1110 | H | H | H | H | acetyl | H | H |
| 1111 | H | H | H | H | acetyl | H | acetyl |
| 1112 | H | H | H | H | acetyl | acetyl | H |
| 1113 | H | H | H | H | acetyl | acetyl | acetyl |
| 1114 | H | H | H | acetyl | H | H | H |
| 1115 | H | H | H | acetyl | H | H | acetyl |
| 1116 | H | H | H | acetyl | H | acetyl | H |
| 1117 | H | H | H | acetyl | H | acetyl | acetyl |
| 1118 | H | H | H | acetyl | acetyl | H | H |
| 1119 | H | H | H | acetyl | acetyl | H | acetyl |
| 1120 | H | H | H | acetyl | acetyl | acetyl | H |
| 1121 | H | H | H | acetyl | acetyl | acetyl | acetyl |
| 1122 | H | H | acetyl | H | H | H | H |
| 1123 | H | H | acetyl | H | H | H | acetyl |
| 1124 | H | H | acetyl | H | H | acetyl | H |
| 1125 | H | H | acetyl | H | H | acetyl | acetyl |
| 1126 | H | H | acetyl | H | acetyl | H | H |
| 1127 | H | H | acetyl | H | acetyl | H | acetyl |
| 1128 | H | H | acetyl | H | acetyl | acetyl | H |
| 1129 | H | H | acetyl | H | acetyl | acetyl | acetyl |
| 1130 | H | H | acetyl | acetyl | H | H | H |
| 1131 | H | H | acetyl | acetyl | H | H | acetyl |
| 1132 | H | H | acetyl | acetyl | H | acetyl | H |
| 1133 | H | H | acetyl | acetyl | H | acetyl | acetyl |
| 1134 | H | H | acetyl | acetyl | acetyl | H | H |
| 1135 | H | H | acetyl | acetyl | acetyl | H | acetyl |
| 1136 | H | H | acetyl | acetyl | acetyl | acetyl | H |
| 1137 | H | H | acetyl | acetyl | acetyl | acetyl | acetyl |
| 1138 | H | acetyl | H | H | H | H | H |
| 1139 | H | acetyl | H | H | H | H | acetyl |
| 1140 | H | acetyl | H | H | H | acetyl | H |
| 1141 | H | acetyl | H | H | H | acetyl | acetyl |
| 1142 | H | acetyl | H | H | acetyl | H | H |
| 1143 | H | acetyl | H | H | acetyl | H | acetyl |
| 1144 | H | acetyl | H | H | acetyl | acetyl | H |
| 1145 | H | acetyl | H | H | acetyl | acetyl | acetyl |
| 1146 | H | acetyl | H | acetyl | H | H | H |
| 1147 | H | acetyl | H | acetyl | H | H | acetyl |
| 1148 | H | acetyl | H | acetyl | H | acetyl | H |
| 1149 | H | acetyl | H | acetyl | H | acetyl | acetyl |
| 1150 | H | acetyl | H | acetyl | acetyl | H | H |
| 1151 | H | acetyl | H | acetyl | acetyl | H | acetyl |
| 1152 | H | acetyl | H | acetyl | acetyl | acetyl | H |
| 1153 | H | acetyl | H | acetyl | acetyl | acetyl | acetyl |
| 1154 | H | acetyl | acetyl | H | H | H | H |
| 1155 | H | acetyl | acetyl | H | H | H | acetyl |
| 1156 | H | acetyl | acetyl | H | H | acetyl | H |
| 1157 | H | acetyl | acetyl | H | H | acetyl | acetyl |
| 1158 | H | acetyl | acetyl | H | acetyl | H | H |
| 1159 | H | acetyl | acetyl | H | acetyl | H | acetyl |
| 1160 | H | acetyl | acetyl | H | acetyl | acetyl | H |
| 1161 | H | acetyl | acetyl | H | acetyl | acetyl | acetyl |
| 1162 | H | acetyl | acetyl | acetyl | H | H | H |
| 1163 | H | acetyl | acetyl | acetyl | H | H | acetyl |
| 1164 | H | acetyl | acetyl | acetyl | H | acetyl | H |
| 1165 | H | acetyl | acetyl | acetyl | H | acetyl | acetyl |
| 1166 | H | acetyl | acetyl | acetyl | acetyl | H | H |
| 1167 | H | acetyl | acetyl | acetyl | acetyl | H | acetyl |
| 1168 | H | acetyl | acetyl | acetyl | acetyl | acetyl | H |
| 1169 | H | acetyl | acetyl | acetyl | acetyl | acetyl | acetyl |
| 1170 | acetyl | H | H | H | H | H | H |
| 1171 | acetyl | H | H | H | H | H | acetyl |
| 1172 | acetyl | H | H | H | H | acetyl | H |
| 1173 | acetyl | H | H | H | H | acetyl | acetyl |
| 1174 | acetyl | H | H | H | acetyl | H | H |
| 1175 | acetyl | H | H | H | acetyl | H | acetyl |
| 1176 | acetyl | H | H | H | acetyl | acetyl | H |
| 1177 | acetyl | H | H | H | acetyl | acetyl | acetyl |
| 1178 | acetyl | H | H | acetyl | H | H | H |
| 1179 | acetyl | H | H | acetyl | H | H | acetyl |
| 1180 | acetyl | H | H | acetyl | H | acetyl | H |
| 1181 | acetyl | H | H | acetyl | H | acetyl | acetyl |
| 1182 | acetyl | H | H | acetyl | acetyl | H | H |
| 1183 | acetyl | H | H | acetyl | acetyl | H | acetyl |
| 1184 | acetyl | H | H | acetyl | acetyl | acetyl | H |
| 1185 | acetyl | H | H | acetyl | acetyl | acetyl | acetyl |
| 1186 | acetyl | H | acetyl | H | H | H | H |
| 1187 | acetyl | H | acetyl | H | H | H | acetyl |
| 1188 | acetyl | H | acetyl | H | H | acetyl | H |
| 1189 | acetyl | H | acetyl | H | H | acetyl | acetyl |
| 1190 | acetyl | H | acetyl | H | acetyl | H | H |
| 1191 | acetyl | H | acetyl | H | acetyl | H | acetyl |
| 1192 | acetyl | H | acetyl | H | acetyl | acetyl | H |
| 1193 | acetyl | H | acetyl | H | acetyl | acetyl | acetyl |
| 1194 | acetyl | H | acetyl | acetyl | H | H | H |
| 1195 | acetyl | H | acetyl | acetyl | H | H | acetyl |
| 1196 | acetyl | H | acetyl | acetyl | H | acetyl | H |
| 1197 | acetyl | H | acetyl | acetyl | H | acetyl | acetyl |
| 1198 | acetyl | H | acetyl | acetyl | acetyl | H | H |
| 1199 | acetyl | H | acetyl | acetyl | acetyl | H | acetyl |
| 1200 | acetyl | H | acetyl | acetyl | acetyl | acetyl | H |
| 1201 | acetyl | H | acetyl | acetyl | acetyl | acetyl | acetyl |
| 1202 | acetyl | acetyl | H | H | H | H | H |
| 1203 | acetyl | acetyl | H | H | H | H | acetyl |
| 1204 | acetyl | acetyl | H | H | H | acetyl | H |
| 1205 | acetyl | acetyl | H | H | H | acetyl | acetyl |
| 1206 | acetyl | acetyl | H | H | acetyl | H | H |
| 1207 | acetyl | acetyl | H | H | acetyl | H | acetyl |
| 1208 | acetyl | acetyl | H | H | acetyl | acetyl | H |
| 1209 | acetyl | acetyl | H | H | acetyl | acetyl | acetyl |
| 1210 | acetyl | acetyl | H | acetyl | H | H | H |
| 1211 | acetyl | acetyl | H | acetyl | H | H | acetyl |
| 1212 | acetyl | acetyl | H | acetyl | H | acetyl | H |
| 1213 | acetyl | acetyl | H | acetyl | H | acetyl | acetyl |
| 1214 | acetyl | acetyl | H | acetyl | acetyl | H | H |
| 1215 | acetyl | acetyl | H | acetyl | acetyl | H | acetyl |
| 1216 | acetyl | acetyl | H | acetyl | acetyl | acetyl | H |
| 1217 | acetyl | acetyl | H | acetyl | acetyl | acetyl | acetyl |
| 1218 | acetyl | acetyl | acetyl | H | H | H | H |
| 1219 | acetyl | acetyl | acetyl | H | H | H | acetyl |
| 1220 | acetyl | acetyl | acetyl | H | H | acetyl | H |
| 1221 | acetyl | acetyl | acetyl | H | H | acetyl | acetyl |
| 1222 | acetyl | acetyl | acetyl | H | acetyl | H | H |
| 1223 | acetyl | acetyl | acetyl | H | acetyl | H | acetyl |
| 1224 | acetyl | acetyl | acetyl | H | acetyl | acetyl | H |
| 1225 | acetyl | acetyl | acetyl | H | acetyl | acetyl | acetyl |
| 1226 | acetyl | acetyl | acetyl | acetyl | H | H | H |
| 1227 | acetyl | acetyl | acetyl | acetyl | H | H | acetyl |
| 1228 | acetyl | acetyl | acetyl | acetyl | H | acetyl | H |
| 1229 | acetyl | acetyl | acetyl | acetyl | H | acetyl | acetyl |
| 1230 | acetyl | acetyl | acetyl | acetyl | acetyl | H | H |
| 1231 | acetyl | acetyl | acetyl | acetyl | acetyl | H | acetyl |
| 1232 | acetyl | acetyl | acetyl | acetyl | acetyl | acetyl | H |

TABLE 7
(Decasaccharides) (Y is either H or a carrier)

| Compound | R2 | n1, R1 | n2, R1 | n3, R1 | n4, R1 | n5, R1 | n6, R1 | n7, R1 | n8, R1 | n9, R1 |
|---|---|---|---|---|---|---|---|---|---|---|
| 1997 | H | H | H | H | H | H | H | H | H | acetyl |
| 1998 | H | H | H | H | H | H | H | H | acetyl | H |
| 1999 | H | H | H | H | H | H | H | H | acetyl | acetyl |
| 2000 | H | H | H | H | H | H | H | acetyl | H | H |
| 2001 | H | H | H | H | H | H | H | acetyl | H | acetyl |
| 2002 | H | H | H | H | H | H | H | acetyl | acetyl | H |
| 2003 | H | H | H | H | H | H | H | acetyl | acetyl | acetyl |
| 2004 | H | H | H | H | H | H | acetyl | H | H | H |
| 2005 | H | H | H | H | H | H | acetyl | H | H | acetyl |
| 2006 | H | H | H | H | H | H | acetyl | H | acetyl | H |
| 2007 | H | H | H | H | H | H | acetyl | H | acetyl | acetyl |
| 2008 | H | H | H | H | H | H | acetyl | acetyl | H | H |
| 2009 | H | H | H | H | H | H | acetyl | acetyl | H | acetyl |
| 2010 | H | H | H | H | H | H | acetyl | acetyl | acetyl | H |
| 2011 | H | H | H | H | H | H | acetyl | acetyl | acetyl | acetyl |
| 2012 | H | H | H | H | H | acetyl | H | H | H | H |
| 2013 | H | H | H | H | H | acetyl | H | H | H | acetyl |
| 2014 | H | H | H | H | H | acetyl | H | H | acetyl | H |
| 2015 | H | H | H | H | H | acetyl | H | H | acetyl | acetyl |
| 2016 | H | H | H | H | H | acetyl | H | acetyl | H | H |
| 2017 | H | H | H | H | H | acetyl | H | acetyl | H | acetyl |
| 2018 | H | H | H | H | H | acetyl | H | acetyl | acetyl | H |
| 2019 | H | H | H | H | H | acetyl | H | acetyl | acetyl | acetyl |
| 2020 | H | H | H | H | H | acetyl | acetyl | H | H | H |
| 2021 | H | H | H | H | H | acetyl | acetyl | H | H | acetyl |
| 2022 | H | H | H | H | H | acetyl | acetyl | H | acetyl | H |
| 2023 | H | H | H | H | H | acetyl | acetyl | H | acetyl | acetyl |
| 2024 | H | H | H | H | H | acetyl | acetyl | acetyl | H | H |
| 2025 | H | H | H | H | H | acetyl | acetyl | acetyl | H | acetyl |
| 2026 | H | H | H | H | H | acetyl | acetyl | acetyl | acetyl | H |
| 2027 | H | H | H | H | H | acetyl | acetyl | acetyl | acetyl | acetyl |
| 2028 | H | H | H | H | acetyl | H | H | H | H | H |
| 2029 | H | H | H | H | acetyl | H | H | H | H | acetyl |
| 2030 | H | H | H | H | acetyl | H | H | H | acetyl | H |
| 2031 | H | H | H | H | acetyl | H | H | H | acetyl | acetyl |
| 2032 | H | H | H | H | acetyl | H | H | acetyl | H | H |
| 2033 | H | H | H | H | acetyl | H | H | acetyl | H | acetyl |
| 2034 | H | H | H | H | acetyl | H | H | acetyl | acetyl | H |
| 2035 | H | H | H | H | acetyl | H | H | acetyl | acetyl | acetyl |
| 2036 | H | H | H | H | acetyl | H | acetyl | H | H | H |
| 2037 | H | H | H | H | acetyl | H | acetyl | H | H | acetyl |
| 2038 | H | H | H | H | acetyl | H | acetyl | H | acetyl | H |
| 2039 | H | H | H | H | acetyl | H | acetyl | H | acetyl | acetyl |
| 2040 | H | H | H | H | acetyl | H | acetyl | acetyl | H | H |
| 2041 | H | H | H | H | acetyl | H | acetyl | acetyl | H | acetyl |
| 2042 | H | H | H | H | acetyl | H | acetyl | acetyl | acetyl | H |
| 2043 | H | H | H | H | acetyl | H | acetyl | acetyl | acetyl | acetyl |
| 2044 | H | H | H | H | acetyl | acetyl | H | H | H | H |
| 2045 | H | H | H | H | acetyl | acetyl | H | H | H | acetyl |
| 2046 | H | H | H | H | acetyl | acetyl | H | H | acetyl | H |
| 2047 | H | H | H | H | acetyl | acetyl | H | H | acetyl | acetyl |
| 2048 | H | H | H | H | acetyl | acetyl | H | acetyl | H | H |
| 2049 | H | H | H | H | acetyl | acetyl | H | acetyl | H | acetyl |
| 2050 | H | H | H | H | acetyl | acetyl | H | acetyl | acetyl | H |
| 2051 | H | H | H | H | acetyl | acetyl | H | acetyl | acetyl | acetyl |
| 2052 | H | H | H | H | acetyl | acetyl | acetyl | H | H | H |
| 2053 | H | H | H | H | acetyl | acetyl | acetyl | H | H | acetyl |
| 2054 | H | H | H | H | acetyl | acetyl | acetyl | H | acetyl | H |
| 2055 | H | H | H | H | acetyl | acetyl | acetyl | H | acetyl | acetyl |
| 2056 | H | H | H | H | acetyl | acetyl | acetyl | acetyl | H | H |
| 2057 | H | H | H | H | acetyl | acetyl | acetyl | acetyl | H | acetyl |
| 2058 | H | H | H | H | acetyl | acetyl | acetyl | acetyl | acetyl | H |
| 2059 | H | H | H | H | acetyl | acetyl | acetyl | acetyl | acetyl | acetyl |
| 2060 | H | H | H | acetyl | H | H | H | H | H | H |
| 2061 | H | H | H | acetyl | H | H | H | H | H | acetyl |
| 2062 | H | H | H | acetyl | H | H | H | H | acetyl | H |
| 2063 | H | H | H | acetyl | H | H | H | H | acetyl | acetyl |
| 2064 | H | H | H | acetyl | H | H | H | acetyl | H | H |
| 2065 | H | H | H | acetyl | H | H | H | acetyl | H | acetyl |
| 2066 | H | H | H | acetyl | H | H | H | acetyl | acetyl | H |
| 2067 | H | H | H | acetyl | H | H | H | acetyl | acetyl | acetyl |
| 2068 | H | H | H | acetyl | H | H | acetyl | H | H | H |
| 2069 | H | H | H | acetyl | H | H | acetyl | H | H | acetyl |
| 2070 | H | H | H | acetyl | H | H | acetyl | H | acetyl | H |
| 2071 | H | H | H | acetyl | H | H | acetyl | H | acetyl | acetyl |
| 2072 | H | H | H | acetyl | H | H | acetyl | acetyl | H | H |
| 2073 | H | H | H | acetyl | H | H | acetyl | acetyl | H | acetyl |
| 2074 | H | H | H | acetyl | H | H | acetyl | acetyl | acetyl | H |
| 2075 | H | H | H | acetyl | H | H | acetyl | acetyl | acetyl | acetyl |
| 2076 | H | H | H | acetyl | H | acetyl | H | H | H | H |
| 2077 | H | H | H | acetyl | H | acetyl | H | H | H | acetyl |
| 2078 | H | H | H | acetyl | H | acetyl | H | H | acetyl | H |
| 2079 | H | H | H | acetyl | H | acetyl | H | H | acetyl | acetyl |
| 2080 | H | H | H | acetyl | H | acetyl | H | acetyl | H | H |
| 2081 | H | H | H | acetyl | H | acetyl | H | acetyl | H | acetyl |
| 2082 | H | H | H | acetyl | H | acetyl | H | acetyl | acetyl | H |
| 2083 | H | H | H | acetyl | H | acetyl | H | acetyl | acetyl | acetyl |
| 2084 | H | H | H | acetyl | H | acetyl | acetyl | H | H | H |
| 2085 | H | H | H | acetyl | H | acetyl | acetyl | H | H | acetyl |
| 2086 | H | H | H | acetyl | H | acetyl | acetyl | H | acetyl | H |
| 2087 | H | H | H | acetyl | H | acetyl | acetyl | H | acetyl | acetyl |
| 2088 | H | H | H | acetyl | H | acetyl | acetyl | acetyl | H | H |
| 2089 | H | H | H | acetyl | H | acetyl | acetyl | acetyl | H | acetyl |
| 2090 | H | H | H | acetyl | H | acetyl | acetyl | acetyl | acetyl | H |
| 2091 | H | H | H | acetyl | H | acetyl | acetyl | acetyl | acetyl | acetyl |
| 2092 | H | H | H | acetyl | acetyl | H | H | H | H | H |
| 2093 | H | H | H | acetyl | acetyl | H | H | H | H | acetyl |
| 2094 | H | H | H | acetyl | acetyl | H | H | H | acetyl | H |
| 2095 | H | H | H | acetyl | acetyl | H | H | H | acetyl | acetyl |
| 2096 | H | H | H | acetyl | acetyl | H | H | acetyl | H | H |
| 2097 | H | H | H | acetyl | acetyl | H | H | acetyl | H | acetyl |
| 2098 | H | H | H | acetyl | acetyl | H | H | acetyl | acetyl | H |
| 2099 | H | H | H | acetyl | acetyl | H | H | acetyl | acetyl | acetyl |
| 2100 | H | H | H | acetyl | acetyl | H | acetyl | H | H | H |
| 2101 | H | H | H | acetyl | acetyl | H | acetyl | H | H | acetyl |
| 2102 | H | H | H | acetyl | acetyl | H | acetyl | H | acetyl | H |
| 2103 | H | H | H | acetyl | acetyl | H | acetyl | H | acetyl | acetyl |
| 2104 | H | H | H | acetyl | acetyl | H | acetyl | acetyl | H | H |
| 2105 | H | H | H | acetyl | acetyl | H | acetyl | acetyl | H | acetyl |
| 2106 | H | H | H | acetyl | acetyl | H | acetyl | acetyl | acetyl | H |
| 2107 | H | H | H | acetyl | acetyl | H | acetyl | acetyl | acetyl | acetyl |
| 2108 | H | H | H | acetyl | acetyl | acetyl | H | H | H | H |
| 2109 | H | H | H | acetyl | acetyl | acetyl | H | H | H | acetyl |
| 2110 | H | H | H | acetyl | acetyl | acetyl | H | H | acetyl | H |
| 2111 | H | H | H | acetyl | acetyl | acetyl | H | H | acetyl | acetyl |
| 2112 | H | H | H | acetyl | acetyl | acetyl | H | acetyl | H | H |
| 2113 | H | H | H | acetyl | acetyl | acetyl | H | acetyl | H | acetyl |
| 2114 | H | H | H | acetyl | acetyl | acetyl | H | acetyl | acetyl | H |
| 2115 | H | H | H | acetyl | acetyl | acetyl | H | acetyl | acetyl | acetyl |
| 2116 | H | H | H | acetyl | acetyl | acetyl | acetyl | H | H | H |
| 2117 | H | H | H | acetyl | acetyl | acetyl | acetyl | H | H | acetyl |
| 2118 | H | H | H | acetyl | acetyl | acetyl | acetyl | H | acetyl | H |
| 2119 | H | H | H | acetyl | acetyl | acetyl | acetyl | H | acetyl | acetyl |
| 2120 | H | H | H | acetyl | acetyl | acetyl | acetyl | acetyl | H | H |
| 2121 | H | H | H | acetyl | acetyl | acetyl | acetyl | acetyl | H | acetyl |
| 2122 | H | H | H | acetyl | acetyl | acetyl | acetyl | acetyl | acetyl | H |
| 2123 | H | H | H | acetyl | acetyl | acetyl | acetyl | acetyl | acetyl | acetyl |
| 2124 | H | H | acetyl | H | H | H | H | H | H | H |
| 2125 | H | H | acetyl | H | H | H | H | H | H | acetyl |
| 2126 | H | H | acetyl | H | H | H | H | H | acetyl | H |
| 2127 | H | H | acetyl | H | H | H | H | H | acetyl | acetyl |
| 2128 | H | H | acetyl | H | H | H | H | acetyl | H | H |
| 2129 | H | H | acetyl | H | H | H | H | acetyl | H | acetyl |
| 2130 | H | H | acetyl | H | H | H | H | acetyl | acetyl | H |
| 2131 | H | H | acetyl | H | H | H | H | acetyl | acetyl | acetyl |
| 2132 | H | H | acetyl | H | H | H | acetyl | H | H | H |
| 2133 | H | H | acetyl | H | H | H | acetyl | H | H | acetyl |
| 2134 | H | H | acetyl | H | H | H | acetyl | H | acetyl | H |
| 2135 | H | H | acetyl | H | H | H | acetyl | H | acetyl | acetyl |
| 2136 | H | H | acetyl | H | H | H | acetyl | acetyl | H | H |
| 2137 | H | H | acetyl | H | H | H | acetyl | acetyl | H | acetyl |
| 2138 | H | H | acetyl | H | H | H | acetyl | acetyl | acetyl | H |
| 2139 | H | H | acetyl | H | H | H | acetyl | acetyl | acetyl | acetyl |
| 2140 | H | H | acetyl | H | H | acetyl | H | H | H | H |
| 2141 | H | H | acetyl | H | H | acetyl | H | H | H | acetyl |
| 2142 | H | H | acetyl | H | H | acetyl | H | H | acetyl | H |
| 2143 | H | H | acetyl | H | H | acetyl | H | H | acetyl | acetyl |
| 2144 | H | H | acetyl | H | H | acetyl | H | acetyl | H | H |
| 2145 | H | H | acetyl | H | H | acetyl | H | acetyl | H | acetyl |
| 2146 | H | H | acetyl | H | H | acetyl | H | acetyl | acetyl | H |
| 2147 | H | H | acetyl | H | H | acetyl | H | acetyl | acetyl | acetyl |
| 2148 | H | H | acetyl | H | H | acetyl | acetyl | H | H | H |
| 2149 | H | H | acetyl | H | H | acetyl | acetyl | H | H | acetyl |
| 2150 | H | H | acetyl | H | H | acetyl | acetyl | H | acetyl | H |
| 2151 | H | H | acetyl | H | H | acetyl | acetyl | H | acetyl | acetyl |
| 2152 | H | H | acetyl | H | H | acetyl | acetyl | acetyl | H | H |
| 2153 | H | H | acetyl | H | H | acetyl | acetyl | acetyl | H | acetyl |
| 2154 | H | H | acetyl | H | H | acetyl | acetyl | acetyl | acetyl | H |
| 2155 | H | H | acetyl | H | H | acetyl | acetyl | acetyl | acetyl | acetyl |
| 2156 | H | H | acetyl | H | acetyl | H | H | H | H | H |
| 2157 | H | H | acetyl | H | acetyl | H | H | H | H | acetyl |
| 2158 | H | H | acetyl | H | acetyl | H | H | H | acetyl | H |
| 2159 | H | H | acetyl | H | acetyl | H | H | H | acetyl | acetyl |
| 2160 | H | H | acetyl | H | acetyl | H | H | acetyl | H | H |
| 2161 | H | H | acetyl | H | acetyl | H | H | acetyl | H | acetyl |
| 2162 | H | H | acetyl | H | acetyl | H | H | acetyl | acetyl | H |
| 2163 | H | H | acetyl | H | acetyl | H | H | acetyl | acetyl | acetyl |
| 2164 | H | H | acetyl | H | acetyl | H | acetyl | H | H | H |
| 2165 | H | H | acetyl | H | acetyl | H | acetyl | H | H | acetyl |
| 2166 | H | H | acetyl | H | acetyl | H | acetyl | H | acetyl | H |
| 2167 | H | H | acetyl | H | acetyl | H | acetyl | H | acetyl | acetyl |
| 2168 | H | H | acetyl | H | acetyl | H | acetyl | acetyl | H | H |
| 2169 | H | H | acetyl | H | acetyl | H | acetyl | acetyl | H | acetyl |
| 2170 | H | H | acetyl | H | acetyl | H | acetyl | acetyl | acetyl | H |
| 2171 | H | H | acetyl | H | acetyl | H | acetyl | acetyl | acetyl | acetyl |
| 2172 | H | H | acetyl | H | acetyl | acetyl | H | H | H | H |
| 2173 | H | H | acetyl | H | acetyl | acetyl | H | H | H | acetyl |
| 2174 | H | H | acetyl | H | acetyl | acetyl | H | H | acetyl | H |
| 2175 | H | H | acetyl | H | acetyl | acetyl | H | H | acetyl | acetyl |
| 2176 | H | H | acetyl | H | acetyl | acetyl | H | acetyl | H | H |
| 2177 | H | H | acetyl | H | acetyl | acetyl | H | acetyl | H | acetyl |
| 2178 | H | H | acetyl | H | acetyl | acetyl | H | acetyl | acetyl | H |
| 2179 | H | H | acetyl | H | acetyl | acetyl | H | acetyl | acetyl | acetyl |
| 2180 | H | H | acetyl | H | acetyl | acetyl | acetyl | H | H | H |
| 2181 | H | H | acetyl | H | acetyl | acetyl | acetyl | H | H | acetyl |
| 2182 | H | H | acetyl | H | acetyl | acetyl | acetyl | H | acetyl | H |
| 2183 | H | H | acetyl | H | acetyl | acetyl | acetyl | H | acetyl | acetyl |
| 2184 | H | H | acetyl | H | acetyl | acetyl | acetyl | acetyl | H | H |
| 2185 | H | H | acetyl | H | acetyl | acetyl | acetyl | acetyl | H | acetyl |
| 2186 | H | H | acetyl | H | acetyl | acetyl | acetyl | acetyl | acetyl | H |
| 2187 | H | H | acetyl | H | acetyl | acetyl | acetyl | acetyl | acetyl | acetyl |
| 2188 | H | H | acetyl | acetyl | H | H | H | H | H | H |
| 2189 | H | H | acetyl | acetyl | H | H | H | H | H | acetyl |
| 2190 | H | H | acetyl | acetyl | H | H | H | H | acetyl | H |
| 2191 | H | H | acetyl | acetyl | H | H | H | H | acetyl | acetyl |
| 2192 | H | H | acetyl | acetyl | H | H | H | acetyl | H | H |
| 2193 | H | H | acetyl | acetyl | H | H | H | acetyl | H | acetyl |
| 2194 | H | H | acetyl | acetyl | H | H | H | acetyl | acetyl | H |
| 2195 | H | H | acetyl | acetyl | H | H | H | acetyl | acetyl | acetyl |
| 2196 | H | H | acetyl | acetyl | H | H | acetyl | H | H | H |
| 2197 | H | H | acetyl | acetyl | H | H | acetyl | H | H | acetyl |
| 2198 | H | H | acetyl | acetyl | H | H | acetyl | H | acetyl | H |
| 2199 | H | H | acetyl | acetyl | H | H | acetyl | H | acetyl | acetyl |
| 2200 | H | H | acetyl | acetyl | H | H | acetyl | acetyl | H | H |
| 2201 | H | H | acetyl | acetyl | H | H | acetyl | acetyl | H | acetyl |
| 2202 | H | H | acetyl | acetyl | H | H | acetyl | acetyl | acetyl | H |
| 2203 | H | H | acetyl | acetyl | H | H | acetyl | acetyl | acetyl | acetyl |
| 2204 | H | H | acetyl | acetyl | H | acetyl | H | H | H | H |
| 2205 | H | H | acetyl | acetyl | H | acetyl | H | H | H | acetyl |
| 2206 | H | H | acetyl | acetyl | H | acetyl | H | H | acetyl | H |
| 2207 | H | H | acetyl | acetyl | H | acetyl | H | H | acetyl | acetyl |
| 2208 | H | H | acetyl | acetyl | H | acetyl | H | acetyl | H | H |
| 2209 | H | H | acetyl | acetyl | H | acetyl | H | acetyl | H | acetyl |
| 2210 | H | H | acetyl | acetyl | H | acetyl | H | acetyl | acetyl | H |
| 2211 | H | H | acetyl | acetyl | H | acetyl | H | acetyl | acetyl | acetyl |
| 2212 | H | H | acetyl | acetyl | H | acetyl | acetyl | H | H | H |
| 2213 | H | H | acetyl | acetyl | H | acetyl | acetyl | H | H | acetyl |
| 2214 | H | H | acetyl | acetyl | H | acetyl | acetyl | H | acetyl | H |
| 2215 | H | H | acetyl | acetyl | H | acetyl | acetyl | H | acetyl | acetyl |
| 2216 | H | H | acetyl | acetyl | H | acetyl | acetyl | acetyl | H | H |
| 2217 | H | H | acetyl | acetyl | H | acetyl | acetyl | acetyl | H | acetyl |
| 2218 | H | H | acetyl | acetyl | H | acetyl | acetyl | acetyl | acetyl | H |
| 2219 | H | H | acetyl | acetyl | H | acetyl | acetyl | acetyl | acetyl | acetyl |
| 2220 | H | H | acetyl | acetyl | acetyl | H | H | H | H | H |
| 2221 | H | H | acetyl | acetyl | acetyl | H | H | H | H | acetyl |
| 2222 | H | H | acetyl | acetyl | acetyl | H | H | H | acetyl | H |
| 2223 | H | H | acetyl | acetyl | acetyl | H | H | H | acetyl | acetyl |
| 2224 | H | H | acetyl | acetyl | acetyl | H | H | acetyl | H | H |
| 2225 | H | H | acetyl | acetyl | acetyl | H | H | acetyl | H | acetyl |
| 2226 | H | H | acetyl | acetyl | acetyl | H | H | acetyl | acetyl | H |
| 2227 | H | H | acetyl | acetyl | acetyl | H | H | acetyl | acetyl | acetyl |
| 2228 | H | H | acetyl | acetyl | acetyl | H | acetyl | H | H | H |
| 2229 | H | H | acetyl | acetyl | acetyl | H | acetyl | H | H | acetyl |
| 2230 | H | H | acetyl | acetyl | acetyl | H | acetyl | H | acetyl | H |
| 2231 | H | H | acetyl | acetyl | acetyl | H | acetyl | H | acetyl | acetyl |
| 2232 | H | H | acetyl | acetyl | acetyl | H | acetyl | acetyl | H | H |
| 2233 | H | H | acetyl | acetyl | acetyl | H | acetyl | acetyl | H | acetyl |
| 2234 | H | H | acetyl | acetyl | acetyl | H | acetyl | acetyl | acetyl | H |
| 2235 | H | H | acetyl | acetyl | acetyl | H | acetyl | acetyl | acetyl | acetyl |
| 2236 | H | H | acetyl | acetyl | acetyl | acetyl | H | H | H | H |
| 2237 | H | H | acetyl | acetyl | acetyl | acetyl | H | H | H | acetyl |
| 2238 | H | H | acetyl | acetyl | acetyl | acetyl | H | H | acetyl | H |
| 2239 | H | H | acetyl | acetyl | acetyl | acetyl | H | H | acetyl | acetyl |
| 2240 | H | H | acetyl | acetyl | acetyl | acetyl | H | acetyl | H | H |
| 2241 | H | H | acetyl | acetyl | acetyl | acetyl | H | acetyl | H | acetyl |
| 2242 | H | H | acetyl | acetyl | acetyl | acetyl | H | acetyl | acetyl | H |
| 2243 | H | H | acetyl | acetyl | acetyl | acetyl | H | acetyl | acetyl | acetyl |
| 2244 | H | H | acetyl | acetyl | acetyl | acetyl | acetyl | H | H | H |
| 2245 | H | H | acetyl | acetyl | acetyl | acetyl | acetyl | H | H | acetyl |
| 2246 | H | H | acetyl | acetyl | acetyl | acetyl | acetyl | H | acetyl | H |
| 2247 | H | H | acetyl | acetyl | acetyl | acetyl | acetyl | H | acetyl | acetyl |
| 2248 | H | H | acetyl | acetyl | acetyl | acetyl | acetyl | acetyl | H | H |
| 2249 | H | H | acetyl | acetyl | acetyl | acetyl | acetyl | acetyl | H | acetyl |
| 2250 | H | H | acetyl | acetyl | acetyl | acetyl | acetyl | acetyl | acetyl | H |
| 2251 | H | H | acetyl | acetyl | acetyl | acetyl | acetyl | acetyl | acetyl | acetyl |
| 2252 | H | acetyl | H | H | H | H | H | H | H | H |
| 2253 | H | acetyl | H | H | H | H | H | H | H | acetyl |
| 2254 | H | acetyl | H | H | H | H | H | H | acetyl | H |
| 2255 | H | acetyl | H | H | H | H | H | H | acetyl | acetyl |
| 2256 | H | acetyl | H | H | H | H | H | acetyl | H | H |
| 2257 | H | acetyl | H | H | H | H | H | acetyl | H | acetyl |
| 2258 | H | acetyl | H | H | H | H | H | acetyl | acetyl | H |
| 2259 | H | acetyl | H | H | H | H | H | acetyl | acetyl | acetyl |
| 2260 | H | acetyl | H | H | H | H | acetyl | H | H | H |
| 2261 | H | acetyl | H | H | H | H | acetyl | H | H | acetyl |
| 2262 | H | acetyl | H | H | H | H | acetyl | H | acetyl | H |
| 2263 | H | acetyl | H | H | H | H | acetyl | H | acetyl | acetyl |
| 2264 | H | acetyl | H | H | H | H | acetyl | acetyl | H | H |
| 2265 | H | acetyl | H | H | H | H | acetyl | acetyl | H | acetyl |
| 2266 | H | acetyl | H | H | H | H | acetyl | acetyl | acetyl | H |
| 2267 | H | acetyl | H | H | H | H | acetyl | acetyl | acetyl | acetyl |
| 2268 | H | acetyl | H | H | H | acetyl | H | H | H | H |
| 2269 | H | acetyl | H | H | H | acetyl | H | H | H | acetyl |
| 2270 | H | acetyl | H | H | H | acetyl | H | H | acetyl | H |
| 2271 | H | acetyl | H | H | H | acetyl | H | H | acetyl | acetyl |
| 2272 | H | acetyl | H | H | H | acetyl | H | acetyl | H | H |
| 2273 | H | acetyl | H | H | H | acetyl | H | acetyl | H | acetyl |
| 2274 | H | acetyl | H | H | H | acetyl | H | acetyl | acetyl | H |
| 2275 | H | acetyl | H | H | H | acetyl | H | acetyl | acetyl | acetyl |
| 2276 | H | acetyl | H | H | H | acetyl | acetyl | H | H | H |
| 2277 | H | acetyl | H | H | H | acetyl | acetyl | H | H | acetyl |
| 2278 | H | acetyl | H | H | H | acetyl | acetyl | H | acetyl | H |
| 2279 | H | acetyl | H | H | H | acetyl | acetyl | H | acetyl | acetyl |
| 2280 | H | acetyl | H | H | H | acetyl | acetyl | acetyl | H | H |
| 2281 | H | acetyl | H | H | H | acetyl | acetyl | acetyl | H | acetyl |
| 2282 | H | acetyl | H | H | H | acetyl | acetyl | acetyl | acetyl | H |
| 2283 | H | acetyl | H | H | H | acetyl | acetyl | acetyl | acetyl | acetyl |
| 2284 | H | acetyl | H | H | acetyl | H | H | H | H | H |
| 2285 | H | acetyl | H | H | acetyl | H | H | H | H | acetyl |
| 2286 | H | acetyl | H | H | acetyl | H | H | H | acetyl | H |
| 2287 | H | acetyl | H | H | acetyl | H | H | H | acetyl | acetyl |
| 2288 | H | acetyl | H | H | acetyl | H | H | acetyl | H | H |
| 2289 | H | acetyl | H | H | acetyl | H | H | acetyl | H | acetyl |
| 2290 | H | acetyl | H | H | acetyl | H | H | acetyl | acetyl | H |
| 2291 | H | acetyl | H | H | acetyl | H | H | acetyl | acetyl | acetyl |
| 2292 | H | acetyl | H | H | acetyl | H | acetyl | H | H | H |
| 2293 | H | acetyl | H | H | acetyl | H | acetyl | H | H | acetyl |
| 2294 | H | acetyl | H | H | acetyl | H | acetyl | H | acetyl | H |
| 2295 | H | acetyl | H | H | acetyl | H | acetyl | H | acetyl | acetyl |
| 2296 | H | acetyl | H | H | acetyl | H | acetyl | acetyl | H | H |
| 2297 | H | acetyl | H | H | acetyl | H | acetyl | acetyl | H | acetyl |
| 2298 | H | acetyl | H | H | acetyl | H | acetyl | acetyl | acetyl | H |
| 2299 | H | acetyl | H | H | acetyl | H | acetyl | acetyl | acetyl | acetyl |
| 2300 | H | acetyl | H | H | acetyl | acetyl | H | H | H | H |
| 2301 | H | acetyl | H | H | acetyl | acetyl | H | H | H | acetyl |
| 2302 | H | acetyl | H | H | acetyl | acetyl | H | H | acetyl | H |
| 2303 | H | acetyl | H | H | acetyl | acetyl | H | H | acetyl | acetyl |
| 2304 | H | acetyl | H | H | acetyl | acetyl | H | acetyl | H | H |
| 2305 | H | acetyl | H | H | acetyl | acetyl | H | acetyl | H | acetyl |
| 2306 | H | acetyl | H | H | acetyl | acetyl | H | acetyl | acetyl | H |
| 2307 | H | acetyl | H | H | acetyl | acetyl | H | acetyl | acetyl | acetyl |
| 2308 | H | acetyl | H | H | acetyl | acetyl | acetyl | H | H | H |
| 2309 | H | acetyl | H | H | acetyl | acetyl | acetyl | H | H | acetyl |
| 2310 | H | acetyl | H | H | acetyl | acetyl | acetyl | H | acetyl | H |
| 2311 | H | acetyl | H | H | acetyl | acetyl | acetyl | H | acetyl | acetyl |
| 2312 | H | acetyl | H | H | acetyl | acetyl | acetyl | acetyl | H | H |
| 2313 | H | acetyl | H | H | acetyl | acetyl | acetyl | acetyl | H | acetyl |
| 2314 | H | acetyl | H | H | acetyl | acetyl | acetyl | acetyl | acetyl | H |
| 2315 | H | acetyl | H | H | acetyl | acetyl | acetyl | acetyl | acetyl | acetyl |
| 2316 | H | acetyl | H | acetyl | H | H | H | H | H | H |
| 2317 | H | acetyl | H | acetyl | H | H | H | H | H | acetyl |
| 2318 | H | acetyl | H | acetyl | H | H | H | H | acetyl | H |
| 2319 | H | acetyl | H | acetyl | H | H | H | H | acetyl | acetyl |
| 2320 | H | acetyl | H | acetyl | H | H | H | acetyl | H | H |
| 2321 | H | acetyl | H | acetyl | H | H | H | acetyl | H | acetyl |
| 2322 | H | acetyl | H | acetyl | H | H | H | acetyl | acetyl | H |
| 2323 | H | acetyl | H | acetyl | H | H | H | acetyl | acetyl | acetyl |
| 2324 | H | acetyl | H | acetyl | H | H | acetyl | H | H | H |
| 2325 | H | acetyl | H | acetyl | H | H | acetyl | H | H | acetyl |
| 2326 | H | acetyl | H | acetyl | H | H | acetyl | H | acetyl | H |
| 2327 | H | acetyl | H | acetyl | H | H | acetyl | H | acetyl | acetyl |
| 2328 | H | acetyl | H | acetyl | H | H | acetyl | acetyl | H | H |
| 2329 | H | acetyl | H | acetyl | H | H | acetyl | acetyl | H | acetyl |
| 2330 | H | acetyl | H | acetyl | H | H | acetyl | acetyl | acetyl | H |
| 2331 | H | acetyl | H | acetyl | H | H | acetyl | acetyl | acetyl | acetyl |
| 2332 | H | acetyl | H | acetyl | H | acetyl | H | H | H | H |
| 2333 | H | acetyl | H | acetyl | H | acetyl | H | H | H | acetyl |
| 2334 | H | acetyl | H | acetyl | H | acetyl | H | H | acetyl | H |
| 2335 | H | acetyl | H | acetyl | H | acetyl | H | H | acetyl | acetyl |
| 2336 | H | acetyl | H | acetyl | H | acetyl | H | acetyl | H | H |
| 2337 | H | acetyl | H | acetyl | H | acetyl | H | acetyl | H | acetyl |
| 2338 | H | acetyl | H | acetyl | H | acetyl | H | acetyl | acetyl | H |
| 2339 | H | acetyl | H | acetyl | H | acetyl | H | acetyl | acetyl | acetyl |
| 2340 | H | acetyl | H | acetyl | H | acetyl | acetyl | H | H | H |
| 2341 | H | acetyl | H | acetyl | H | acetyl | acetyl | H | H | acetyl |
| 2342 | H | acetyl | H | acetyl | H | acetyl | acetyl | H | acetyl | H |
| 2343 | H | acetyl | H | acetyl | H | acetyl | acetyl | H | acetyl | acetyl |
| 2344 | H | acetyl | H | acetyl | H | acetyl | acetyl | acetyl | H | H |
| 2345 | H | acetyl | H | acetyl | H | acetyl | acetyl | acetyl | H | acetyl |
| 2346 | H | acetyl | H | acetyl | H | acetyl | acetyl | acetyl | acetyl | H |
| 2347 | H | acetyl | H | acetyl | H | acetyl | acetyl | acetyl | acetyl | acetyl |
| 2348 | H | acetyl | H | acetyl | acetyl | H | H | H | H | H |
| 2349 | H | acetyl | H | acetyl | acetyl | H | H | H | H | acetyl |
| 2350 | H | acetyl | H | acetyl | acetyl | H | H | H | acetyl | H |
| 2351 | H | acetyl | H | acetyl | acetyl | H | H | H | acetyl | acetyl |
| 2352 | H | acetyl | H | acetyl | acetyl | H | H | acetyl | H | H |
| 2353 | H | acetyl | H | acetyl | acetyl | H | H | acetyl | H | acetyl |
| 2354 | H | acetyl | H | acetyl | acetyl | H | H | acetyl | acetyl | H |
| 2355 | H | acetyl | H | acetyl | acetyl | H | H | acetyl | acetyl | acetyl |
| 2356 | H | acetyl | H | acetyl | acetyl | H | acetyl | H | H | H |
| 2357 | H | acetyl | H | acetyl | acetyl | H | acetyl | H | H | acetyl |
| 2358 | H | acetyl | H | acetyl | acetyl | H | acetyl | H | acetyl | H |
| 2359 | H | acetyl | H | acetyl | acetyl | H | acetyl | H | acetyl | acetyl |
| 2360 | H | acetyl | H | acetyl | acetyl | H | acetyl | acetyl | H | H |
| 2361 | H | acetyl | H | acetyl | acetyl | H | acetyl | acetyl | H | acetyl |
| 2362 | H | acetyl | H | acetyl | acetyl | H | acetyl | acetyl | acetyl | H |
| 2363 | H | acetyl | H | acetyl | acetyl | H | acetyl | acetyl | acetyl | acetyl |
| 2364 | H | acetyl | H | acetyl | acetyl | acetyl | H | H | H | H |
| 2365 | H | acetyl | H | acetyl | acetyl | acetyl | H | H | H | acetyl |
| 2366 | H | acetyl | H | acetyl | acetyl | acetyl | H | H | acetyl | H |
| 2367 | H | acetyl | H | acetyl | acetyl | acetyl | H | H | acetyl | acetyl |
| 2368 | H | acetyl | H | acetyl | acetyl | acetyl | H | acetyl | H | H |
| 2369 | H | acetyl | H | acetyl | acetyl | acetyl | H | acetyl | H | acetyl |
| 2370 | H | acetyl | H | acetyl | acetyl | acetyl | H | acetyl | acetyl | H |
| 2371 | H | acetyl | H | acetyl | acetyl | acetyl | H | acetyl | acetyl | acetyl |
| 2372 | H | acetyl | H | acetyl | acetyl | acetyl | acetyl | H | H | H |
| 2373 | H | acetyl | H | acetyl | acetyl | acetyl | acetyl | H | H | acetyl |
| 2374 | H | acetyl | H | acetyl | acetyl | acetyl | acetyl | H | acetyl | H |
| 2375 | H | acetyl | H | acetyl | acetyl | acetyl | acetyl | H | acetyl | acetyl |
| 2376 | H | acetyl | H | acetyl | acetyl | acetyl | acetyl | acetyl | H | H |
| 2377 | H | acetyl | H | acetyl | acetyl | acetyl | acetyl | acetyl | H | acetyl |
| 2378 | H | acetyl | H | acetyl | acetyl | acetyl | acetyl | acetyl | acetyl | H |
| 2379 | H | acetyl | H | acetyl | acetyl | acetyl | acetyl | acetyl | acetyl | acetyl |
| 2380 | H | acetyl | acetyl | H | H | H | H | H | H | H |
| 2381 | H | acetyl | acetyl | H | H | H | H | H | H | acetyl |
| 2382 | H | acetyl | acetyl | H | H | H | H | H | acetyl | H |
| 2383 | H | acetyl | acetyl | H | H | H | H | H | acetyl | acetyl |
| 2384 | H | acetyl | acetyl | H | H | H | H | acetyl | H | H |
| 2385 | H | acetyl | acetyl | H | H | H | H | acetyl | H | acetyl |
| 2386 | H | acetyl | acetyl | H | H | H | H | acetyl | acetyl | H |
| 2387 | H | acetyl | acetyl | H | H | H | H | acetyl | acetyl | acetyl |
| 2388 | H | acetyl | acetyl | H | H | H | acetyl | H | H | H |
| 2389 | H | acetyl | acetyl | H | H | H | acetyl | H | H | acetyl |
| 2390 | H | acetyl | acetyl | H | H | H | acetyl | H | acetyl | H |
| 2391 | H | acetyl | acetyl | H | H | H | acetyl | H | acetyl | acetyl |
| 2392 | H | acetyl | acetyl | H | H | H | acetyl | acetyl | H | H |
| 2393 | H | acetyl | acetyl | H | H | H | acetyl | acetyl | H | acetyl |
| 2394 | H | acetyl | acetyl | H | H | H | acetyl | acetyl | acetyl | H |
| 2395 | H | acetyl | acetyl | H | H | H | acetyl | acetyl | acetyl | acetyl |
| 2396 | H | acetyl | acetyl | H | H | acetyl | H | H | H | H |
| 2397 | H | acetyl | acetyl | H | H | acetyl | H | H | H | acetyl |
| 2398 | H | acetyl | acetyl | H | H | acetyl | H | H | acetyl | H |
| 2399 | H | acetyl | acetyl | H | H | acetyl | H | H | acetyl | acetyl |
| 2400 | H | acetyl | acetyl | H | H | acetyl | H | acetyl | H | H |
| 2401 | H | acetyl | acetyl | H | H | acetyl | H | acetyl | H | acetyl |
| 2402 | H | acetyl | acetyl | H | H | acetyl | H | acetyl | acetyl | H |
| 2403 | H | acetyl | acetyl | H | H | acetyl | H | acetyl | acetyl | acetyl |
| 2404 | H | acetyl | acetyl | H | H | acetyl | acetyl | H | H | H |
| 2405 | H | acetyl | acetyl | H | H | acetyl | acetyl | H | H | acetyl |
| 2406 | H | acetyl | acetyl | H | H | acetyl | acetyl | H | acetyl | H |
| 2407 | H | acetyl | acetyl | H | H | acetyl | acetyl | H | acetyl | acetyl |
| 2408 | H | acetyl | acetyl | H | H | acetyl | acetyl | acetyl | H | H |
| 2409 | H | acetyl | acetyl | H | H | acetyl | acetyl | acetyl | H | acetyl |
| 2410 | H | acetyl | acetyl | H | H | acetyl | acetyl | acetyl | acetyl | H |
| 2411 | H | acetyl | acetyl | H | H | acetyl | acetyl | acetyl | acetyl | acetyl |
| 2412 | H | acetyl | acetyl | H | acetyl | H | H | H | H | H |
| 2413 | H | acetyl | acetyl | H | acetyl | H | H | H | H | acetyl |
| 2414 | H | acetyl | acetyl | H | acetyl | H | H | H | acetyl | H |
| 2415 | H | acetyl | acetyl | H | acetyl | H | H | H | acetyl | acetyl |
| 2416 | H | acetyl | acetyl | H | acetyl | H | H | acetyl | H | H |
| 2417 | H | acetyl | acetyl | H | acetyl | H | H | acetyl | H | acetyl |
| 2418 | H | acetyl | acetyl | H | acetyl | H | H | acetyl | acetyl | H |
| 2419 | H | acetyl | acetyl | H | acetyl | H | H | acetyl | acetyl | acetyl |
| 2420 | H | acetyl | acetyl | H | acetyl | H | acetyl | H | H | H |
| 2421 | H | acetyl | acetyl | H | acetyl | H | acetyl | H | H | acetyl |
| 2422 | H | acetyl | acetyl | H | acetyl | H | acetyl | H | acetyl | H |
| 2423 | H | acetyl | acetyl | H | acetyl | H | acetyl | H | acetyl | acetyl |
| 2424 | H | acetyl | acetyl | H | acetyl | H | acetyl | acetyl | H | H |
| 2425 | H | acetyl | acetyl | H | acetyl | H | acetyl | acetyl | H | acetyl |
| 2426 | H | acetyl | acetyl | H | acetyl | H | acetyl | acetyl | acetyl | H |
| 2427 | H | acetyl | acetyl | H | acetyl | H | acetyl | acetyl | acetyl | acetyl |
| 2428 | H | acetyl | acetyl | H | acetyl | acetyl | H | H | H | H |
| 2429 | H | acetyl | acetyl | H | acetyl | acetyl | H | H | H | acetyl |
| 2430 | H | acetyl | acetyl | H | acetyl | acetyl | H | H | acetyl | H |
| 2431 | H | acetyl | acetyl | H | acetyl | acetyl | H | H | acetyl | acetyl |
| 2432 | H | acetyl | acetyl | H | acetyl | acetyl | H | acetyl | H | H |
| 2433 | H | acetyl | acetyl | H | acetyl | acetyl | H | acetyl | H | acetyl |
| 2434 | H | acetyl | acetyl | H | acetyl | acetyl | H | acetyl | acetyl | H |
| 2435 | H | acetyl | acetyl | H | acetyl | acetyl | H | acetyl | acetyl | acetyl |
| 2436 | H | acetyl | acetyl | H | acetyl | acetyl | acetyl | H | H | H |
| 2437 | H | acetyl | acetyl | H | acetyl | acetyl | acetyl | H | H | acetyl |
| 2438 | H | acetyl | acetyl | H | acetyl | acetyl | acetyl | H | acetyl | H |
| 2439 | H | acetyl | acetyl | H | acetyl | acetyl | acetyl | H | acetyl | acetyl |
| 2440 | H | acetyl | acetyl | H | acetyl | acetyl | acetyl | acetyl | H | H |
| 2441 | H | acetyl | acetyl | H | acetyl | acetyl | acetyl | acetyl | H | acetyl |
| 2442 | H | acetyl | acetyl | H | acetyl | acetyl | acetyl | acetyl | acetyl | H |
| 2443 | H | acetyl | acetyl | H | acetyl | acetyl | acetyl | acetyl | acetyl | acetyl |
| 2444 | H | acetyl | acetyl | acetyl | H | H | H | H | H | H |
| 2445 | H | acetyl | acetyl | acetyl | H | H | H | H | H | acetyl |
| 2446 | H | acetyl | acetyl | acetyl | H | H | H | H | acetyl | H |
| 2447 | H | acetyl | acetyl | acetyl | H | H | H | H | acetyl | acetyl |
| 2448 | H | acetyl | acetyl | acetyl | H | H | H | acetyl | H | H |
| 2449 | H | acetyl | acetyl | acetyl | H | H | H | acetyl | H | acetyl |
| 2450 | H | acetyl | acetyl | acetyl | H | H | H | acetyl | acetyl | H |
| 2451 | H | acetyl | acetyl | acetyl | H | H | H | acetyl | acetyl | acetyl |
| 2452 | H | acetyl | acetyl | acetyl | H | H | acetyl | H | H | H |
| 2453 | H | acetyl | acetyl | acetyl | H | H | acetyl | H | H | acetyl |
| 2454 | H | acetyl | acetyl | acetyl | H | H | acetyl | H | acetyl | H |
| 2455 | H | acetyl | acetyl | acetyl | H | H | acetyl | H | acetyl | acetyl |
| 2456 | H | acetyl | acetyl | acetyl | H | H | acetyl | acetyl | H | H |
| 2457 | H | acetyl | acetyl | acetyl | H | H | acetyl | acetyl | H | acetyl |
| 2458 | H | acetyl | acetyl | acetyl | H | H | acetyl | acetyl | acetyl | H |
| 2459 | H | acetyl | acetyl | acetyl | H | H | acetyl | acetyl | acetyl | acetyl |
| 2460 | H | acetyl | acetyl | acetyl | H | acetyl | H | H | H | H |
| 2461 | H | acetyl | acetyl | acetyl | H | acetyl | H | H | H | acetyl |
| 2462 | H | acetyl | acetyl | acetyl | H | acetyl | H | H | acetyl | H |
| 2463 | H | acetyl | acetyl | acetyl | H | acetyl | H | H | acetyl | acetyl |
| 2464 | H | acetyl | acetyl | acetyl | H | acetyl | H | acetyl | H | H |
| 2465 | H | acetyl | acetyl | acetyl | H | acetyl | H | acetyl | H | acetyl |
| 2466 | H | acetyl | acetyl | acetyl | H | acetyl | H | acetyl | acetyl | H |
| 2467 | H | acetyl | acetyl | acetyl | H | acetyl | H | acetyl | acetyl | acetyl |
| 2468 | H | acetyl | acetyl | acetyl | H | acetyl | acetyl | H | H | H |
| 2469 | H | acetyl | acetyl | acetyl | H | acetyl | acetyl | H | H | acetyl |
| 2470 | H | acetyl | acetyl | acetyl | H | acetyl | acetyl | H | acetyl | H |
| 2471 | H | acetyl | acetyl | acetyl | H | acetyl | acetyl | H | acetyl | acetyl |
| 2472 | H | acetyl | acetyl | acetyl | H | acetyl | acetyl | acetyl | H | H |
| 2473 | H | acetyl | acetyl | acetyl | H | acetyl | acetyl | acetyl | H | acetyl |
| 2474 | H | acetyl | acetyl | acetyl | H | acetyl | acetyl | acetyl | acetyl | H |
| 2475 | H | acetyl | acetyl | acetyl | H | acetyl | acetyl | acetyl | acetyl | acetyl |
| 2476 | H | acetyl | acetyl | acetyl | acetyl | H | H | H | H | H |
| 2477 | H | acetyl | acetyl | acetyl | acetyl | H | H | H | H | acetyl |
| 2478 | H | acetyl | acetyl | acetyl | acetyl | H | H | H | acetyl | H |
| 2479 | H | acetyl | acetyl | acetyl | acetyl | H | H | H | acetyl | acetyl |
| 2480 | H | acetyl | acetyl | acetyl | acetyl | H | H | acetyl | H | H |
| 2481 | H | acetyl | acetyl | acetyl | acetyl | H | H | acetyl | H | acetyl |
| 2482 | H | acetyl | acetyl | acetyl | acetyl | H | H | acetyl | acetyl | H |
| 2483 | H | acetyl | acetyl | acetyl | acetyl | H | H | acetyl | acetyl | acetyl |
| 2484 | H | acetyl | acetyl | acetyl | acetyl | H | acetyl | H | H | H |
| 2485 | H | acetyl | acetyl | acetyl | acetyl | H | acetyl | H | H | acetyl |
| 2486 | H | acetyl | acetyl | acetyl | acetyl | H | acetyl | H | acetyl | H |
| 2487 | H | acetyl | acetyl | acetyl | acetyl | H | acetyl | H | acetyl | acetyl |
| 2488 | H | acetyl | acetyl | acetyl | acetyl | H | acetyl | acetyl | H | H |
| 2489 | H | acetyl | acetyl | acetyl | acetyl | H | acetyl | acetyl | H | acetyl |
| 2490 | H | acetyl | acetyl | acetyl | acetyl | H | acetyl | acetyl | acetyl | H |
| 2491 | H | acetyl | acetyl | acetyl | acetyl | H | acetyl | acetyl | acetyl | acetyl |
| 2492 | H | acetyl | acetyl | acetyl | acetyl | acetyl | H | H | H | H |
| 2493 | H | acetyl | acetyl | acetyl | acetyl | acetyl | H | H | H | acetyl |
| 2494 | H | acetyl | acetyl | acetyl | acetyl | acetyl | H | H | acetyl | H |
| 2495 | H | acetyl | acetyl | acetyl | acetyl | acetyl | H | H | acetyl | acetyl |
| 2496 | H | acetyl | acetyl | acetyl | acetyl | acetyl | H | acetyl | H | H |
| 2497 | H | acetyl | acetyl | acetyl | acetyl | acetyl | H | acetyl | H | acetyl |
| 2498 | H | acetyl | acetyl | acetyl | acetyl | acetyl | H | acetyl | acetyl | H |
| 2499 | H | acetyl | acetyl | acetyl | acetyl | acetyl | H | acetyl | acetyl | acetyl |
| 2500 | H | acetyl | acetyl | acetyl | acetyl | acetyl | acetyl | H | H | H |
| 2501 | H | acetyl | acetyl | acetyl | acetyl | acetyl | acetyl | H | H | acetyl |
| 2502 | H | acetyl | acetyl | acetyl | acetyl | acetyl | acetyl | H | acetyl | H |
| 2503 | H | acetyl | acetyl | acetyl | acetyl | acetyl | acetyl | H | acetyl | acetyl |
| 2504 | H | acetyl | acetyl | acetyl | acetyl | acetyl | acetyl | acetyl | H | H |
| 2505 | H | acetyl | acetyl | acetyl | acetyl | acetyl | acetyl | acetyl | H | acetyl |
| 2506 | H | acetyl | acetyl | acetyl | acetyl | acetyl | acetyl | acetyl | acetyl | H |
| 2507 | H | acetyl | acetyl | acetyl | acetyl | acetyl | acetyl | acetyl | acetyl | acetyl |
| 2508 | acetyl | H | H | H | H | H | H | H | H | H |
| 2509 | acetyl | H | H | H | H | H | H | H | H | acetyl |
| 2510 | acetyl | H | H | H | H | H | H | H | acetyl | H |
| 2511 | acetyl | H | H | H | H | H | H | H | acetyl | acetyl |
| 2512 | acetyl | H | H | H | H | H | H | acetyl | H | H |
| 2513 | acetyl | H | H | H | H | H | H | acetyl | H | acetyl |
| 2514 | acetyl | H | H | H | H | H | H | acetyl | acetyl | H |
| 2515 | acetyl | H | H | H | H | H | H | acetyl | acetyl | acetyl |
| 2516 | acetyl | H | H | H | H | H | acetyl | H | H | H |
| 2517 | acetyl | H | H | H | H | H | acetyl | H | H | acetyl |
| 2518 | acetyl | H | H | H | H | H | acetyl | H | acetyl | H |
| 2519 | acetyl | H | H | H | H | H | acetyl | H | acetyl | acetyl |
| 2520 | acetyl | H | H | H | H | H | acetyl | acetyl | H | H |
| 2521 | acetyl | H | H | H | H | H | acetyl | acetyl | H | acetyl |
| 2522 | acetyl | H | H | H | H | H | acetyl | acetyl | acetyl | H |
| 2523 | acetyl | H | H | H | H | H | acetyl | acetyl | acetyl | acetyl |
| 2524 | acetyl | H | H | H | H | acetyl | H | H | H | H |
| 2525 | acetyl | H | H | H | H | acetyl | H | H | H | acetyl |
| 2526 | acetyl | H | H | H | H | acetyl | H | H | acetyl | H |
| 2527 | acetyl | H | H | H | H | acetyl | H | H | acetyl | acetyl |
| 2528 | acetyl | H | H | H | H | acetyl | H | acetyl | H | H |
| 2529 | acetyl | H | H | H | H | acetyl | H | acetyl | H | acetyl |
| 2530 | acetyl | H | H | H | H | acetyl | H | acetyl | acetyl | H |
| 2531 | acetyl | H | H | H | H | acetyl | H | acetyl | acetyl | acetyl |
| 2532 | acetyl | H | H | H | H | acetyl | acetyl | H | H | H |
| 2533 | acetyl | H | H | H | H | acetyl | acetyl | H | H | acetyl |
| 2534 | acetyl | H | H | H | H | acetyl | acetyl | H | acetyl | H |
| 2535 | acetyl | H | H | H | H | acetyl | acetyl | H | acetyl | acetyl |
| 2536 | acetyl | H | H | H | H | acetyl | acetyl | acetyl | H | H |
| 2537 | acetyl | H | H | H | H | acetyl | acetyl | acetyl | H | acetyl |
| 2538 | acetyl | H | H | H | H | acetyl | acetyl | acetyl | acetyl | H |
| 2539 | acetyl | H | H | H | H | acetyl | acetyl | acetyl | acetyl | acetyl |
| 2540 | acetyl | H | H | H | acetyl | H | H | H | H | H |
| 2541 | acetyl | H | H | H | acetyl | H | H | H | H | acetyl |
| 2542 | acetyl | H | H | H | acetyl | H | H | H | acetyl | H |
| 2543 | acetyl | H | H | H | acetyl | H | H | H | acetyl | acetyl |
| 2544 | acetyl | H | H | H | acetyl | H | H | acetyl | H | H |
| 2545 | acetyl | H | H | H | acetyl | H | H | acetyl | H | acetyl |
| 2546 | acetyl | H | H | H | acetyl | H | H | acetyl | acetyl | H |
| 2547 | acetyl | H | H | H | acetyl | H | H | acetyl | acetyl | acetyl |
| 2548 | acetyl | H | H | H | acetyl | H | acetyl | H | H | H |
| 2549 | acetyl | H | H | H | acetyl | H | acetyl | H | H | acetyl |
| 2550 | acetyl | H | H | H | acetyl | H | acetyl | H | acetyl | H |
| 2551 | acetyl | H | H | H | acetyl | H | acetyl | H | acetyl | acetyl |
| 2552 | acetyl | H | H | H | acetyl | H | acetyl | acetyl | H | H |
| 2553 | acetyl | H | H | H | acetyl | H | acetyl | acetyl | H | acetyl |
| 2554 | acetyl | H | H | H | acetyl | H | acetyl | acetyl | acetyl | H |
| 2555 | acetyl | H | H | H | acetyl | H | acetyl | acetyl | acetyl | acetyl |
| 2556 | acetyl | H | H | H | acetyl | acetyl | H | H | H | H |
| 2557 | acetyl | H | H | H | acetyl | acetyl | H | H | H | acetyl |
| 2558 | acetyl | H | H | H | acetyl | acetyl | H | H | acetyl | H |
| 2559 | acetyl | H | H | H | acetyl | acetyl | H | H | acetyl | acetyl |
| 2560 | acetyl | H | H | H | acetyl | acetyl | H | acetyl | H | H |
| 2561 | acetyl | H | H | H | acetyl | acetyl | H | acetyl | H | acetyl |
| 2562 | acetyl | H | H | H | acetyl | acetyl | H | acetyl | acetyl | H |
| 2563 | acetyl | H | H | H | acetyl | acetyl | H | acetyl | acetyl | acetyl |
| 2564 | acetyl | H | H | H | acetyl | acetyl | acetyl | H | H | H |
| 2565 | acetyl | H | H | H | acetyl | acetyl | acetyl | H | H | acetyl |
| 2566 | acetyl | H | H | H | acetyl | acetyl | acetyl | H | acetyl | H |
| 2567 | acetyl | H | H | H | acetyl | acetyl | acetyl | H | acetyl | acetyl |
| 2568 | acetyl | H | H | H | acetyl | acetyl | acetyl | acetyl | H | H |
| 2569 | acetyl | H | H | H | acetyl | acetyl | acetyl | acetyl | H | acetyl |
| 2570 | acetyl | H | H | H | acetyl | acetyl | acetyl | acetyl | acetyl | H |
| 2571 | acetyl | H | H | H | acetyl | acetyl | acetyl | acetyl | acetyl | acetyl |
| 2572 | acetyl | H | H | acetyl | H | H | H | H | H | H |
| 2573 | acetyl | H | H | acetyl | H | H | H | H | H | acetyl |
| 2574 | acetyl | H | H | acetyl | H | H | H | H | acetyl | H |
| 2575 | acetyl | H | H | acetyl | H | H | H | H | acetyl | acetyl |
| 2576 | acetyl | H | H | acetyl | H | H | H | acetyl | H | H |
| 2577 | acetyl | H | H | acetyl | H | H | H | acetyl | H | acetyl |
| 2578 | acetyl | H | H | acetyl | H | H | H | acetyl | acetyl | H |
| 2579 | acetyl | H | H | acetyl | H | H | H | acetyl | acetyl | acetyl |
| 2580 | acetyl | H | H | acetyl | H | H | acetyl | H | H | H |
| 2581 | acetyl | H | H | acetyl | H | H | acetyl | H | H | acetyl |
| 2582 | acetyl | H | H | acetyl | H | H | acetyl | H | acetyl | H |
| 2583 | acetyl | H | H | acetyl | H | H | acetyl | H | acetyl | acetyl |
| 2584 | acetyl | H | H | acetyl | H | H | acetyl | acetyl | H | H |
| 2585 | acetyl | H | H | acetyl | H | H | acetyl | acetyl | H | acetyl |
| 2586 | acetyl | H | H | acetyl | H | H | acetyl | acetyl | acetyl | H |
| 2587 | acetyl | H | H | acetyl | H | H | acetyl | acetyl | acetyl | acetyl |
| 2588 | acetyl | H | H | acetyl | H | acetyl | H | H | H | H |
| 2589 | acetyl | H | H | acetyl | H | acetyl | H | H | H | acetyl |
| 2590 | acetyl | H | H | acetyl | H | acetyl | H | H | acetyl | H |
| 2591 | acetyl | H | H | acetyl | H | acetyl | H | H | acetyl | acetyl |
| 2592 | acetyl | H | H | acetyl | H | acetyl | H | acetyl | H | H |
| 2593 | acetyl | H | H | acetyl | H | acetyl | H | acetyl | H | acetyl |
| 2594 | acetyl | H | H | acetyl | H | acetyl | H | acetyl | acetyl | H |
| 2595 | acetyl | H | H | acetyl | H | acetyl | H | acetyl | acetyl | acetyl |
| 2596 | acetyl | H | H | acetyl | H | acetyl | acetyl | H | H | H |
| 2597 | acetyl | H | H | acetyl | H | acetyl | acetyl | H | H | acetyl |
| 2598 | acetyl | H | H | acetyl | H | acetyl | acetyl | H | acetyl | H |
| 2599 | acetyl | H | H | acetyl | H | acetyl | acetyl | H | acetyl | acetyl |
| 2600 | acetyl | H | H | acetyl | H | acetyl | acetyl | acetyl | H | H |
| 2601 | acetyl | H | H | acetyl | H | acetyl | acetyl | acetyl | H | acetyl |
| 2602 | acetyl | H | H | acetyl | H | acetyl | acetyl | acetyl | acetyl | H |
| 2603 | acetyl | H | H | acetyl | H | acetyl | acetyl | acetyl | acetyl | acetyl |
| 2604 | acetyl | H | H | acetyl | acetyl | H | H | H | H | H |
| 2605 | acetyl | H | H | acetyl | acetyl | H | H | H | H | acetyl |
| 2606 | acetyl | H | H | acetyl | acetyl | H | H | H | acetyl | H |
| 2607 | acetyl | H | H | acetyl | acetyl | H | H | H | acetyl | acetyl |
| 2608 | acetyl | H | H | acetyl | acetyl | H | H | acetyl | H | H |
| 2609 | acetyl | H | H | acetyl | acetyl | H | H | acetyl | H | acetyl |
| 2610 | acetyl | H | H | acetyl | acetyl | H | H | acetyl | acetyl | H |
| 2611 | acetyl | H | H | acetyl | acetyl | H | H | acetyl | acetyl | acetyl |
| 2612 | acetyl | H | H | acetyl | acetyl | H | acetyl | H | H | H |
| 2613 | acetyl | H | H | acetyl | acetyl | H | acetyl | H | H | acetyl |
| 2614 | acetyl | H | H | acetyl | acetyl | H | acetyl | H | acetyl | H |
| 2615 | acetyl | H | H | acetyl | acetyl | H | acetyl | H | acetyl | acetyl |
| 2616 | acetyl | H | H | acetyl | acetyl | H | acetyl | acetyl | H | H |
| 2617 | acetyl | H | H | acetyl | acetyl | H | acetyl | acetyl | H | acetyl |
| 2618 | acetyl | H | H | acetyl | acetyl | H | acetyl | acetyl | acetyl | H |
| 2619 | acetyl | H | H | acetyl | acetyl | H | acetyl | acetyl | acetyl | acetyl |
| 2620 | acetyl | H | H | acetyl | acetyl | acetyl | H | H | H | H |
| 2621 | acetyl | H | H | acetyl | acetyl | acetyl | H | H | H | acetyl |
| 2622 | acetyl | H | H | acetyl | acetyl | acetyl | H | H | acetyl | H |
| 2623 | acetyl | H | H | acetyl | acetyl | acetyl | H | H | acetyl | acetyl |
| 2624 | acetyl | H | H | acetyl | acetyl | acetyl | H | acetyl | H | H |
| 2625 | acetyl | H | H | acetyl | acetyl | acetyl | H | acetyl | H | acetyl |
| 2626 | acetyl | H | H | acetyl | acetyl | acetyl | H | acetyl | acetyl | H |
| 2627 | acetyl | H | H | acetyl | acetyl | acetyl | H | acetyl | acetyl | acetyl |
| 2628 | acetyl | H | H | acetyl | acetyl | acetyl | acetyl | H | H | H |
| 2629 | acetyl | H | H | acetyl | acetyl | acetyl | acetyl | H | H | acetyl |
| 2630 | acetyl | H | H | acetyl | acetyl | acetyl | acetyl | H | acetyl | H |
| 2631 | acetyl | H | H | acetyl | acetyl | acetyl | acetyl | H | acetyl | acetyl |
| 2632 | acetyl | H | H | acetyl | acetyl | acetyl | acetyl | acetyl | H | H |
| 2633 | acetyl | H | H | acetyl | acetyl | acetyl | acetyl | acetyl | H | acetyl |
| 2634 | acetyl | H | H | acetyl | acetyl | acetyl | acetyl | acetyl | acetyl | H |
| 2635 | acetyl | H | H | acetyl | acetyl | acetyl | acetyl | acetyl | acetyl | acetyl |
| 2636 | acetyl | H | acetyl | H | H | H | H | H | H | H |
| 2637 | acetyl | H | acetyl | H | H | H | H | H | H | acetyl |
| 2638 | acetyl | H | acetyl | H | H | H | H | H | acetyl | H |
| 2639 | acetyl | H | acetyl | H | H | H | H | H | acetyl | acetyl |
| 2640 | acetyl | H | acetyl | H | H | H | H | acetyl | H | H |
| 2641 | acetyl | H | acetyl | H | H | H | H | acetyl | H | acetyl |
| 2642 | acetyl | H | acetyl | H | H | H | H | acetyl | acetyl | H |
| 2643 | acetyl | H | acetyl | H | H | H | H | acetyl | acetyl | acetyl |
| 2644 | acetyl | H | acetyl | H | H | H | acetyl | H | H | H |
| 2645 | acetyl | H | acetyl | H | H | H | acetyl | H | H | acetyl |
| 2646 | acetyl | H | acetyl | H | H | H | acetyl | H | acetyl | H |
| 2647 | acetyl | H | acetyl | H | H | H | acetyl | H | acetyl | acetyl |
| 2648 | acetyl | H | acetyl | H | H | H | acetyl | acetyl | H | H |
| 2649 | acetyl | H | acetyl | H | H | H | acetyl | acetyl | H | acetyl |
| 2650 | acetyl | H | acetyl | H | H | H | acetyl | acetyl | acetyl | H |
| 2651 | acetyl | H | acetyl | H | H | H | acetyl | acetyl | acetyl | acetyl |
| 2652 | acetyl | H | acetyl | H | H | acetyl | H | H | H | H |
| 2653 | acetyl | H | acetyl | H | H | acetyl | H | H | H | acetyl |
| 2654 | acetyl | H | acetyl | H | H | acetyl | H | H | acetyl | H |
| 2655 | acetyl | H | acetyl | H | H | acetyl | H | H | acetyl | acetyl |
| 2656 | acetyl | H | acetyl | H | H | acetyl | H | acetyl | H | H |
| 2657 | acetyl | H | acetyl | H | H | acetyl | H | acetyl | H | acetyl |
| 2658 | acetyl | H | acetyl | H | H | acetyl | H | acetyl | acetyl | H |
| 2659 | acetyl | H | acetyl | H | H | acetyl | H | acetyl | acetyl | acetyl |
| 2660 | acetyl | H | acetyl | H | H | acetyl | acetyl | H | H | H |
| 2661 | acetyl | H | acetyl | H | H | acetyl | acetyl | H | H | acetyl |
| 2662 | acetyl | H | acetyl | H | H | acetyl | acetyl | H | acetyl | H |
| 2663 | acetyl | H | acetyl | H | H | acetyl | acetyl | H | acetyl | acetyl |
| 2664 | acetyl | H | acetyl | H | H | acetyl | acetyl | acetyl | H | H |
| 2665 | acetyl | H | acetyl | H | H | acetyl | acetyl | acetyl | H | acetyl |
| 2666 | acetyl | H | acetyl | H | H | acetyl | acetyl | acetyl | acetyl | H |
| 2667 | acetyl | H | acetyl | H | H | acetyl | acetyl | acetyl | acetyl | acetyl |
| 2668 | acetyl | H | acetyl | H | acetyl | H | H | H | H | H |
| 2669 | acetyl | H | acetyl | H | acetyl | H | H | H | H | acetyl |
| 2670 | acetyl | H | acetyl | H | acetyl | H | H | H | acetyl | H |
| 2671 | acetyl | H | acetyl | H | acetyl | H | H | H | acetyl | acetyl |
| 2672 | acetyl | H | acetyl | H | acetyl | H | H | acetyl | H | H |
| 2673 | acetyl | H | acetyl | H | acetyl | H | H | acetyl | H | acetyl |
| 2674 | acetyl | H | acetyl | H | acetyl | H | H | acetyl | acetyl | H |
| 2675 | acetyl | H | acetyl | H | acetyl | H | H | acetyl | acetyl | acetyl |
| 2676 | acetyl | H | acetyl | H | acetyl | H | acetyl | H | H | H |
| 2677 | acetyl | H | acetyl | H | acetyl | H | acetyl | H | H | acetyl |
| 2678 | acetyl | H | acetyl | H | acetyl | H | acetyl | H | acetyl | H |
| 2679 | acetyl | H | acetyl | H | acetyl | H | acetyl | H | acetyl | acetyl |
| 2680 | acetyl | H | acetyl | H | acetyl | H | acetyl | acetyl | H | H |
| 2681 | acetyl | H | acetyl | H | acetyl | H | acetyl | acetyl | H | acetyl |
| 2682 | acetyl | H | acetyl | H | acetyl | H | acetyl | acetyl | acetyl | H |
| 2683 | acetyl | H | acetyl | H | acetyl | H | acetyl | acetyl | acetyl | acetyl |
| 2684 | acetyl | H | acetyl | H | acetyl | acetyl | H | H | H | H |
| 2685 | acetyl | H | acetyl | H | acetyl | acetyl | H | H | H | acetyl |
| 2686 | acetyl | H | acetyl | H | acetyl | acetyl | H | H | acetyl | H |
| 2687 | acetyl | H | acetyl | H | acetyl | acetyl | H | H | acetyl | acetyl |
| 2688 | acetyl | H | acetyl | H | acetyl | acetyl | H | acetyl | H | H |
| 2689 | acetyl | H | acetyl | H | acetyl | acetyl | H | acetyl | H | acetyl |
| 2690 | acetyl | H | acetyl | H | acetyl | acetyl | H | acetyl | acetyl | H |
| 2691 | acetyl | H | acetyl | H | acetyl | acetyl | H | acetyl | acetyl | acetyl |
| 2692 | acetyl | H | acetyl | H | acetyl | acetyl | acetyl | H | H | H |
| 2693 | acetyl | H | acetyl | H | acetyl | acetyl | acetyl | H | H | acetyl |
| 2694 | acetyl | H | acetyl | H | acetyl | acetyl | acetyl | H | acetyl | H |
| 2695 | acetyl | H | acetyl | H | acetyl | acetyl | acetyl | H | acetyl | acetyl |
| 2696 | acetyl | H | acetyl | H | acetyl | acetyl | acetyl | acetyl | H | H |
| 2697 | acetyl | H | acetyl | H | acetyl | acetyl | acetyl | acetyl | H | acetyl |
| 2698 | acetyl | H | acetyl | H | acetyl | acetyl | acetyl | acetyl | acetyl | H |
| 2699 | acetyl | H | acetyl | H | acetyl | acetyl | acetyl | acetyl | acetyl | acetyl |
| 2700 | acetyl | H | acetyl | acetyl | H | H | H | H | H | H |
| 2701 | acetyl | H | acetyl | acetyl | H | H | H | H | H | acetyl |
| 2702 | acetyl | H | acetyl | acetyl | H | H | H | H | acetyl | H |
| 2703 | acetyl | H | acetyl | acetyl | H | H | H | H | acetyl | acetyl |
| 2704 | acetyl | H | acetyl | acetyl | H | H | H | acetyl | H | H |
| 2705 | acetyl | H | acetyl | acetyl | H | H | H | acetyl | H | acetyl |
| 2706 | acetyl | H | acetyl | acetyl | H | H | H | acetyl | acetyl | H |
| 2707 | acetyl | H | acetyl | acetyl | H | H | H | acetyl | acetyl | acetyl |
| 2708 | acetyl | H | acetyl | acetyl | H | H | acetyl | H | H | H |
| 2709 | acetyl | H | acetyl | acetyl | H | H | acetyl | H | H | acetyl |
| 2710 | acetyl | H | acetyl | acetyl | H | H | acetyl | H | acetyl | H |
| 2711 | acetyl | H | acetyl | acetyl | H | H | acetyl | H | acetyl | acetyl |
| 2712 | acetyl | H | acetyl | acetyl | H | H | acetyl | acetyl | H | H |
| 2713 | acetyl | H | acetyl | acetyl | H | H | acetyl | acetyl | H | acetyl |
| 2714 | acetyl | H | acetyl | acetyl | H | H | acetyl | acetyl | acetyl | H |
| 2715 | acetyl | H | acetyl | acetyl | H | H | acetyl | acetyl | acetyl | acetyl |
| 2716 | acetyl | H | acetyl | acetyl | H | acetyl | H | H | H | H |
| 2717 | acetyl | H | acetyl | acetyl | H | acetyl | H | H | H | acetyl |
| 2718 | acetyl | H | acetyl | acetyl | H | acetyl | H | H | acetyl | H |
| 2719 | acetyl | H | acetyl | acetyl | H | acetyl | H | H | acetyl | acetyl |
| 2720 | acetyl | H | acetyl | acetyl | H | acetyl | H | acetyl | H | H |
| 2721 | acetyl | H | acetyl | acetyl | H | acetyl | H | acetyl | H | acetyl |
| 2722 | acetyl | H | acetyl | acetyl | H | acetyl | H | acetyl | acetyl | H |
| 2723 | acetyl | H | acetyl | acetyl | H | acetyl | H | acetyl | acetyl | acetyl |
| 2724 | acetyl | H | acetyl | acetyl | H | acetyl | acetyl | H | H | H |
| 2725 | acetyl | H | acetyl | acetyl | H | acetyl | acetyl | H | H | acetyl |
| 2726 | acetyl | H | acetyl | acetyl | H | acetyl | acetyl | H | acetyl | H |
| 2727 | acetyl | H | acetyl | acetyl | H | acetyl | acetyl | H | acetyl | acetyl |
| 2728 | acetyl | H | acetyl | acetyl | H | acetyl | acetyl | acetyl | H | H |
| 2729 | acetyl | H | acetyl | acetyl | H | acetyl | acetyl | acetyl | H | acetyl |
| 2730 | acetyl | H | acetyl | acetyl | H | acetyl | acetyl | acetyl | acetyl | H |
| 2731 | acetyl | H | acetyl | acetyl | H | acetyl | acetyl | acetyl | acetyl | acetyl |
| 2732 | acetyl | H | acetyl | acetyl | acetyl | H | H | H | H | H |
| 2733 | acetyl | H | acetyl | acetyl | acetyl | H | H | H | H | acetyl |
| 2734 | acetyl | H | acetyl | acetyl | acetyl | H | H | H | acetyl | H |
| 2735 | acetyl | H | acetyl | acetyl | acetyl | H | H | H | acetyl | acetyl |
| 2736 | acetyl | H | acetyl | acetyl | acetyl | H | H | acetyl | H | H |
| 2737 | acetyl | H | acetyl | acetyl | acetyl | H | H | acetyl | H | acetyl |
| 2738 | acetyl | H | acetyl | acetyl | acetyl | H | H | acetyl | acetyl | H |
| 2739 | acetyl | H | acetyl | acetyl | acetyl | H | H | acetyl | acetyl | acetyl |
| 2740 | acetyl | H | acetyl | acetyl | acetyl | H | acetyl | H | H | H |
| 2741 | acetyl | H | acetyl | acetyl | acetyl | H | acetyl | H | H | acetyl |
| 2742 | acetyl | H | acetyl | acetyl | acetyl | H | acetyl | H | acetyl | H |
| 2743 | acetyl | H | acetyl | acetyl | acetyl | H | acetyl | H | acetyl | acetyl |
| 2744 | acetyl | H | acetyl | acetyl | acetyl | H | acetyl | acetyl | H | H |
| 2745 | acetyl | H | acetyl | acetyl | acetyl | H | acetyl | acetyl | H | acetyl |
| 2746 | acetyl | H | acetyl | acetyl | acetyl | H | acetyl | acetyl | acetyl | H |
| 2747 | acetyl | H | acetyl | acetyl | acetyl | H | acetyl | acetyl | acetyl | acetyl |
| 2748 | acetyl | H | acetyl | acetyl | acetyl | acetyl | H | H | H | H |
| 2749 | acetyl | H | acetyl | acetyl | acetyl | acetyl | H | H | H | acetyl |
| 2750 | acetyl | H | acetyl | acetyl | acetyl | acetyl | H | H | acetyl | H |
| 2751 | acetyl | H | acetyl | acetyl | acetyl | acetyl | H | H | acetyl | acetyl |
| 2752 | acetyl | H | acetyl | acetyl | acetyl | acetyl | H | acetyl | H | H |
| 2753 | acetyl | H | acetyl | acetyl | acetyl | acetyl | H | acetyl | H | acetyl |
| 2754 | acetyl | H | acetyl | acetyl | acetyl | acetyl | H | acetyl | acetyl | H |
| 2755 | acetyl | H | acetyl | acetyl | acetyl | acetyl | H | acetyl | acetyl | acetyl |
| 2756 | acetyl | H | acetyl | acetyl | acetyl | acetyl | acetyl | H | H | H |
| 2757 | acetyl | H | acetyl | acetyl | acetyl | acetyl | acetyl | H | H | acetyl |
| 2758 | acetyl | H | acetyl | acetyl | acetyl | acetyl | acetyl | H | acetyl | H |
| 2759 | acetyl | H | acetyl | acetyl | acetyl | acetyl | acetyl | H | acetyl | acetyl |
| 2760 | acetyl | H | acetyl | acetyl | acetyl | acetyl | acetyl | acetyl | H | H |
| 2761 | acetyl | H | acetyl | acetyl | acetyl | acetyl | acetyl | acetyl | H | acetyl |
| 2762 | acetyl | H | acetyl | acetyl | acetyl | acetyl | acetyl | acetyl | acetyl | H |
| 2763 | acetyl | H | acetyl | acetyl | acetyl | acetyl | acetyl | acetyl | acetyl | acetyl |
| 2764 | acetyl | acetyl | H | H | H | H | H | H | H | H |
| 2765 | acetyl | acetyl | H | H | H | H | H | H | H | acetyl |
| 2766 | acetyl | acetyl | H | H | H | H | H | H | acetyl | H |
| 2767 | acetyl | acetyl | H | H | H | H | H | H | acetyl | acetyl |
| 2768 | acetyl | acetyl | H | H | H | H | H | acetyl | H | H |
| 2769 | acetyl | acetyl | H | H | H | H | H | acetyl | H | acetyl |
| 2770 | acetyl | acetyl | H | H | H | H | H | acetyl | acetyl | H |
| 2771 | acetyl | acetyl | H | H | H | H | H | acetyl | acetyl | acetyl |
| 2772 | acetyl | acetyl | H | H | H | H | acetyl | H | H | H |
| 2773 | acetyl | acetyl | H | H | H | H | acetyl | H | H | acetyl |
| 2774 | acetyl | acetyl | H | H | H | H | acetyl | H | acetyl | H |
| 2775 | acetyl | acetyl | H | H | H | H | acetyl | H | acetyl | acetyl |
| 2776 | acetyl | acetyl | H | H | H | H | acetyl | acetyl | H | H |
| 2777 | acetyl | acetyl | H | H | H | H | acetyl | acetyl | H | acetyl |
| 2778 | acetyl | acetyl | H | H | H | H | acetyl | acetyl | acetyl | H |
| 2779 | acetyl | acetyl | H | H | H | H | acetyl | acetyl | acetyl | acetyl |
| 2780 | acetyl | acetyl | H | H | H | acetyl | H | H | H | H |
| 2781 | acetyl | acetyl | H | H | H | acetyl | H | H | H | acetyl |
| 2782 | acetyl | acetyl | H | H | H | acetyl | H | H | acetyl | H |
| 2783 | acetyl | acetyl | H | H | H | acetyl | H | H | acetyl | acetyl |
| 2784 | acetyl | acetyl | H | H | H | acetyl | H | acetyl | H | H |
| 2785 | acetyl | acetyl | H | H | H | acetyl | H | acetyl | H | acetyl |
| 2786 | acetyl | acetyl | H | H | H | acetyl | H | acetyl | acetyl | H |
| 2787 | acetyl | acetyl | H | H | H | acetyl | H | acetyl | acetyl | acetyl |
| 2788 | acetyl | acetyl | H | H | H | acetyl | acetyl | H | H | H |
| 2789 | acetyl | acetyl | H | H | H | acetyl | acetyl | H | H | acetyl |
| 2790 | acetyl | acetyl | H | H | H | acetyl | acetyl | H | acetyl | H |
| 2791 | acetyl | acetyl | H | H | H | acetyl | acetyl | H | acetyl | acetyl |
| 2792 | acetyl | acetyl | H | H | H | acetyl | acetyl | acetyl | H | H |
| 2793 | acetyl | acetyl | H | H | H | acetyl | acetyl | acetyl | H | acetyl |
| 2794 | acetyl | acetyl | H | H | H | acetyl | acetyl | acetyl | acetyl | H |
| 2795 | acetyl | acetyl | H | H | H | acetyl | acetyl | acetyl | acetyl | acetyl |
| 2796 | acetyl | acetyl | H | H | acetyl | H | H | H | H | H |
| 2797 | acetyl | acetyl | H | H | acetyl | H | H | H | H | acetyl |
| 2798 | acetyl | acetyl | H | H | acetyl | H | H | H | acetyl | H |
| 2799 | acetyl | acetyl | H | H | acetyl | H | H | H | acetyl | acetyl |
| 2800 | acetyl | acetyl | H | H | acetyl | H | H | acetyl | H | H |
| 2801 | acetyl | acetyl | H | H | acetyl | H | H | acetyl | H | acetyl |
| 2802 | acetyl | acetyl | H | H | acetyl | H | H | acetyl | acetyl | H |
| 2803 | acetyl | acetyl | H | H | acetyl | H | H | acetyl | acetyl | acetyl |
| 2804 | acetyl | acetyl | H | H | acetyl | H | acetyl | H | H | H |
| 2805 | acetyl | acetyl | H | H | acetyl | H | acetyl | H | H | acetyl |
| 2806 | acetyl | acetyl | H | H | acetyl | H | acetyl | H | acetyl | H |
| 2807 | acetyl | acetyl | H | H | acetyl | H | acetyl | H | acetyl | acetyl |
| 2808 | acetyl | acetyl | H | H | acetyl | H | acetyl | acetyl | H | H |
| 2809 | acetyl | acetyl | H | H | acetyl | H | acetyl | acetyl | H | acetyl |
| 2810 | acetyl | acetyl | H | H | acetyl | H | acetyl | acetyl | acetyl | H |
| 2811 | acetyl | acetyl | H | H | acetyl | H | acetyl | acetyl | acetyl | acetyl |
| 2812 | acetyl | acetyl | H | H | acetyl | acetyl | H | H | H | H |
| 2813 | acetyl | acetyl | H | H | acetyl | acetyl | H | H | H | acetyl |
| 2814 | acetyl | acetyl | H | H | acetyl | acetyl | H | H | acetyl | H |
| 2815 | acetyl | acetyl | H | H | acetyl | acetyl | H | H | acetyl | acetyl |
| 2816 | acetyl | acetyl | H | H | acetyl | acetyl | H | acetyl | H | H |
| 2817 | acetyl | acetyl | H | H | acetyl | acetyl | H | acetyl | H | acetyl |
| 2818 | acetyl | acetyl | H | H | acetyl | acetyl | H | acetyl | acetyl | H |
| 2819 | acetyl | acetyl | H | H | acetyl | acetyl | H | acetyl | acetyl | acetyl |
| 2820 | acetyl | acetyl | H | H | acetyl | acetyl | acetyl | H | H | H |
| 2821 | acetyl | acetyl | H | H | acetyl | acetyl | acetyl | H | H | acetyl |
| 2822 | acetyl | acetyl | H | H | acetyl | acetyl | acetyl | H | acetyl | H |
| 2823 | acetyl | acetyl | H | H | acetyl | acetyl | acetyl | H | acetyl | acetyl |
| 2824 | acetyl | acetyl | H | H | acetyl | acetyl | acetyl | acetyl | H | H |
| 2825 | acetyl | acetyl | H | H | acetyl | acetyl | acetyl | acetyl | H | acetyl |
| 2826 | acetyl | acetyl | H | H | acetyl | acetyl | acetyl | acetyl | acetyl | H |
| 2827 | acetyl | acetyl | H | H | acetyl | acetyl | acetyl | acetyl | acetyl | acetyl |
| 2828 | acetyl | acetyl | H | acetyl | H | H | H | H | H | H |
| 2829 | acetyl | acetyl | H | acetyl | H | H | H | H | H | acetyl |
| 2830 | acetyl | acetyl | H | acetyl | H | H | H | H | acetyl | H |
| 2831 | acetyl | acetyl | H | acetyl | H | H | H | H | acetyl | acetyl |
| 2832 | acetyl | acetyl | H | acetyl | H | H | H | acetyl | H | H |
| 2833 | acetyl | acetyl | H | acetyl | H | H | H | acetyl | H | acetyl |
| 2834 | acetyl | acetyl | H | acetyl | H | H | H | acetyl | acetyl | H |
| 2835 | acetyl | acetyl | H | acetyl | H | H | H | acetyl | acetyl | acetyl |
| 2836 | acetyl | acetyl | H | acetyl | H | H | acetyl | H | H | H |
| 2837 | acetyl | acetyl | H | acetyl | H | H | acetyl | H | H | acetyl |
| 2838 | acetyl | acetyl | H | acetyl | H | H | acetyl | H | acetyl | H |
| 2839 | acetyl | acetyl | H | acetyl | H | H | acetyl | H | acetyl | acetyl |
| 2840 | acetyl | acetyl | H | acetyl | H | H | acetyl | acetyl | H | H |
| 2841 | acetyl | acetyl | H | acetyl | H | H | acetyl | acetyl | H | acetyl |
| 2842 | acetyl | acetyl | H | acetyl | H | H | acetyl | acetyl | acetyl | H |
| 2843 | acetyl | acetyl | H | acetyl | H | H | acetyl | acetyl | acetyl | acetyl |
| 2844 | acetyl | acetyl | H | acetyl | H | acetyl | H | H | H | H |
| 2845 | acetyl | acetyl | H | acetyl | H | acetyl | H | H | H | acetyl |
| 2846 | acetyl | acetyl | H | acetyl | H | acetyl | H | H | acetyl | H |
| 2847 | acetyl | acetyl | H | acetyl | H | acetyl | H | H | acetyl | acetyl |
| 2848 | acetyl | acetyl | H | acetyl | H | acetyl | H | acetyl | H | H |
| 2849 | acetyl | acetyl | H | acetyl | H | acetyl | H | acetyl | H | acetyl |
| 2850 | acetyl | acetyl | H | acetyl | H | acetyl | H | acetyl | acetyl | H |
| 2851 | acetyl | acetyl | H | acetyl | H | acetyl | H | acetyl | acetyl | acetyl |
| 2852 | acetyl | acetyl | H | acetyl | H | acetyl | acetyl | H | H | H |
| 2853 | acetyl | acetyl | H | acetyl | H | acetyl | acetyl | H | H | acetyl |
| 2854 | acetyl | acetyl | H | acetyl | H | acetyl | acetyl | H | acetyl | H |
| 2855 | acetyl | acetyl | H | acetyl | H | acetyl | acetyl | H | acetyl | acetyl |
| 2856 | acetyl | acetyl | H | acetyl | H | acetyl | acetyl | acetyl | H | H |
| 2857 | acetyl | acetyl | H | acetyl | H | acetyl | acetyl | acetyl | H | acetyl |
| 2858 | acetyl | acetyl | H | acetyl | H | acetyl | acetyl | acetyl | acetyl | H |
| 2859 | acetyl | acetyl | H | acetyl | H | acetyl | acetyl | acetyl | acetyl | acetyl |
| 2860 | acetyl | acetyl | H | acetyl | acetyl | H | H | H | H | H |
| 2861 | acetyl | acetyl | H | acetyl | acetyl | H | H | H | H | acetyl |
| 2862 | acetyl | acetyl | H | acetyl | acetyl | H | H | H | acetyl | H |
| 2863 | acetyl | acetyl | H | acetyl | acetyl | H | H | H | acetyl | acetyl |
| 2864 | acetyl | acetyl | H | acetyl | acetyl | H | H | acetyl | H | H |
| 2865 | acetyl | acetyl | H | acetyl | acetyl | H | H | acetyl | H | acetyl |
| 2866 | acetyl | acetyl | H | acetyl | acetyl | H | H | acetyl | acetyl | H |
| 2867 | acetyl | acetyl | H | acetyl | acetyl | H | H | acetyl | acetyl | acetyl |
| 2868 | acetyl | acetyl | H | acetyl | acetyl | H | acetyl | H | H | H |
| 2869 | acetyl | acetyl | H | acetyl | acetyl | H | acetyl | H | H | acetyl |
| 2870 | acetyl | acetyl | H | acetyl | acetyl | H | acetyl | H | acetyl | H |
| 2871 | acetyl | acetyl | H | acetyl | acetyl | H | acetyl | H | acetyl | acetyl |
| 2872 | acetyl | acetyl | H | acetyl | acetyl | H | acetyl | acetyl | H | H |
| 2873 | acetyl | acetyl | H | acetyl | acetyl | H | acetyl | acetyl | H | acetyl |
| 2874 | acetyl | acetyl | H | acetyl | acetyl | H | acetyl | acetyl | acetyl | H |
| 2875 | acetyl | acetyl | H | acetyl | acetyl | H | acetyl | acetyl | acetyl | acetyl |
| 2876 | acetyl | acetyl | H | acetyl | acetyl | acetyl | H | H | H | H |
| 2877 | acetyl | acetyl | H | acetyl | acetyl | acetyl | H | H | H | acetyl |
| 2878 | acetyl | acetyl | H | acetyl | acetyl | acetyl | H | H | acetyl | H |
| 2879 | acetyl | acetyl | H | acetyl | acetyl | acetyl | H | H | acetyl | acetyl |
| 2880 | acetyl | acetyl | H | acetyl | acetyl | acetyl | H | acetyl | H | H |
| 2881 | acetyl | acetyl | H | acetyl | acetyl | acetyl | H | acetyl | H | acetyl |
| 2882 | acetyl | acetyl | H | acetyl | acetyl | acetyl | H | acetyl | acetyl | H |
| 2883 | acetyl | acetyl | H | acetyl | acetyl | acetyl | H | acetyl | acetyl | acetyl |
| 2884 | acetyl | acetyl | H | acetyl | acetyl | acetyl | acetyl | H | H | H |
| 2885 | acetyl | acetyl | H | acetyl | acetyl | acetyl | acetyl | H | H | acetyl |
| 2886 | acetyl | acetyl | H | acetyl | acetyl | acetyl | acetyl | H | acetyl | H |
| 2887 | acetyl | acetyl | H | acetyl | acetyl | acetyl | acetyl | H | acetyl | acetyl |
| 2888 | acetyl | acetyl | H | acetyl | acetyl | acetyl | acetyl | acetyl | H | H |
| 2889 | acetyl | acetyl | H | acetyl | acetyl | acetyl | acetyl | acetyl | H | acetyl |
| 2890 | acetyl | acetyl | H | acetyl | acetyl | acetyl | acetyl | acetyl | acetyl | H |
| 2891 | acetyl | acetyl | H | acetyl | acetyl | acetyl | acetyl | acetyl | acetyl | acetyl |
| 2892 | acetyl | acetyl | acetyl | H | H | H | H | H | H | H |
| 2893 | acetyl | acetyl | acetyl | H | H | H | H | H | H | acetyl |
| 2894 | acetyl | acetyl | acetyl | H | H | H | H | H | acetyl | H |
| 2895 | acetyl | acetyl | acetyl | H | H | H | H | H | acetyl | acetyl |
| 2896 | acetyl | acetyl | acetyl | H | H | H | H | acetyl | H | H |
| 2897 | acetyl | acetyl | acetyl | H | H | H | H | acetyl | H | acetyl |
| 2898 | acetyl | acetyl | acetyl | H | H | H | H | acetyl | acetyl | H |
| 2899 | acetyl | acetyl | acetyl | H | H | H | H | acetyl | acetyl | acetyl |
| 2900 | acetyl | acetyl | acetyl | H | H | H | acetyl | H | H | H |
| 2901 | acetyl | acetyl | acetyl | H | H | H | acetyl | H | H | acetyl |
| 2902 | acetyl | acetyl | acetyl | H | H | H | acetyl | H | acetyl | H |
| 2903 | acetyl | acetyl | acetyl | H | H | H | acetyl | H | acetyl | acetyl |
| 2904 | acetyl | acetyl | acetyl | H | H | H | acetyl | acetyl | H | H |
| 2905 | acetyl | acetyl | acetyl | H | H | H | acetyl | acetyl | H | acetyl |
| 2906 | acetyl | acetyl | acetyl | H | H | H | acetyl | acetyl | acetyl | H |
| 2907 | acetyl | acetyl | acetyl | H | H | H | acetyl | acetyl | acetyl | acetyl |
| 2908 | acetyl | acetyl | acetyl | H | H | acetyl | H | H | H | H |
| 2909 | acetyl | acetyl | acetyl | H | H | acetyl | H | H | H | acetyl |
| 2910 | acetyl | acetyl | acetyl | H | H | acetyl | H | H | acetyl | H |
| 2911 | acetyl | acetyl | acetyl | H | H | acetyl | H | H | acetyl | acetyl |
| 2912 | acetyl | acetyl | acetyl | H | H | acetyl | H | acetyl | H | H |
| 2913 | acetyl | acetyl | acetyl | H | H | acetyl | H | acetyl | H | acetyl |
| 2914 | acetyl | acetyl | acetyl | H | H | acetyl | H | acetyl | acetyl | H |
| 2915 | acetyl | acetyl | acetyl | H | H | acetyl | H | acetyl | acetyl | acetyl |
| 2916 | acetyl | acetyl | acetyl | H | H | acetyl | acetyl | H | H | H |
| 2917 | acetyl | acetyl | acetyl | H | H | acetyl | acetyl | H | H | acetyl |
| 2918 | acetyl | acetyl | acetyl | H | H | acetyl | acetyl | H | acetyl | H |
| 2919 | acetyl | acetyl | acetyl | H | H | acetyl | acetyl | H | acetyl | acetyl |
| 2920 | acetyl | acetyl | acetyl | H | H | acetyl | acetyl | acetyl | H | H |
| 2921 | acetyl | acetyl | acetyl | H | H | acetyl | acetyl | acetyl | H | acetyl |
| 2922 | acetyl | acetyl | acetyl | H | H | acetyl | acetyl | acetyl | acetyl | H |
| 2923 | acetyl | acetyl | acetyl | H | H | acetyl | acetyl | acetyl | acetyl | acetyl |
| 2924 | acetyl | acetyl | acetyl | H | acetyl | H | H | H | H | H |
| 2925 | acetyl | acetyl | acetyl | H | acetyl | H | H | H | H | acetyl |
| 2926 | acetyl | acetyl | acetyl | H | acetyl | H | H | H | acetyl | H |
| 2927 | acetyl | acetyl | acetyl | H | acetyl | H | H | H | acetyl | acetyl |
| 2928 | acetyl | acetyl | acetyl | H | acetyl | H | H | acetyl | H | H |
| 2929 | acetyl | acetyl | acetyl | H | acetyl | H | H | acetyl | H | acetyl |
| 2930 | acetyl | acetyl | acetyl | H | acetyl | H | H | acetyl | acetyl | H |
| 2931 | acetyl | acetyl | acetyl | H | acetyl | H | H | acetyl | acetyl | acetyl |
| 2932 | acetyl | acetyl | acetyl | H | acetyl | H | acetyl | H | H | H |
| 2933 | acetyl | acetyl | acetyl | H | acetyl | H | acetyl | H | H | acetyl |
| 2934 | acetyl | acetyl | acetyl | H | acetyl | H | acetyl | H | acetyl | H |
| 2935 | acetyl | acetyl | acetyl | H | acetyl | H | acetyl | H | acetyl | acetyl |
| 2936 | acetyl | acetyl | acetyl | H | acetyl | H | acetyl | acetyl | H | H |
| 2937 | acetyl | acetyl | acetyl | H | acetyl | H | acetyl | acetyl | H | acetyl |
| 2938 | acetyl | acetyl | acetyl | H | acetyl | H | acetyl | acetyl | acetyl | H |
| 2939 | acetyl | acetyl | acetyl | H | acetyl | H | acetyl | acetyl | acetyl | acetyl |
| 2940 | acetyl | acetyl | acetyl | H | acetyl | acetyl | H | H | H | H |
| 2941 | acetyl | acetyl | acetyl | H | acetyl | acetyl | H | H | H | acetyl |
| 2942 | acetyl | acetyl | acetyl | H | acetyl | acetyl | H | H | acetyl | H |
| 2943 | acetyl | acetyl | acetyl | H | acetyl | acetyl | H | H | acetyl | acetyl |
| 2944 | acetyl | acetyl | acetyl | H | acetyl | acetyl | H | acetyl | H | H |
| 2945 | acetyl | acetyl | acetyl | H | acetyl | acetyl | H | acetyl | H | acetyl |
| 2946 | acetyl | acetyl | acetyl | H | acetyl | acetyl | H | acetyl | acetyl | H |
| 2947 | acetyl | acetyl | acetyl | H | acetyl | acetyl | H | acetyl | acetyl | acetyl |
| 2948 | acetyl | acetyl | acetyl | H | acetyl | acetyl | acetyl | H | H | H |
| 2949 | acetyl | acetyl | acetyl | H | acetyl | acetyl | acetyl | H | H | acetyl |
| 2950 | acetyl | acetyl | acetyl | H | acetyl | acetyl | acetyl | H | acetyl | H |
| 2951 | acetyl | acetyl | acetyl | H | acetyl | acetyl | acetyl | H | acetyl | acetyl |
| 2952 | acetyl | acetyl | acetyl | H | acetyl | acetyl | acetyl | acetyl | H | H |
| 2953 | acetyl | acetyl | acetyl | H | acetyl | acetyl | acetyl | acetyl | H | acetyl |
| 2954 | acetyl | acetyl | acetyl | H | acetyl | acetyl | acetyl | acetyl | acetyl | H |
| 2955 | acetyl | acetyl | acetyl | H | acetyl | acetyl | acetyl | acetyl | acetyl | acetyl |
| 2956 | acetyl | acetyl | acetyl | acetyl | H | H | H | H | H | H |
| 2957 | acetyl | acetyl | acetyl | acetyl | H | H | H | H | H | acetyl |
| 2958 | acetyl | acetyl | acetyl | acetyl | H | H | H | H | acetyl | H |
| 2959 | acetyl | acetyl | acetyl | acetyl | H | H | H | H | acetyl | acetyl |
| 2960 | acetyl | acetyl | acetyl | acetyl | H | H | H | acetyl | H | H |
| 2961 | acetyl | acetyl | acetyl | acetyl | H | H | H | acetyl | H | acetyl |
| 2962 | acetyl | acetyl | acetyl | acetyl | H | H | H | acetyl | acetyl | H |
| 2963 | acetyl | acetyl | acetyl | acetyl | H | H | H | acetyl | acetyl | acetyl |
| 2964 | acetyl | acetyl | acetyl | acetyl | H | H | acetyl | H | H | H |
| 2965 | acetyl | acetyl | acetyl | acetyl | H | H | acetyl | H | H | acetyl |
| 2966 | acetyl | acetyl | acetyl | acetyl | H | H | acetyl | H | acetyl | H |
| 2967 | acetyl | acetyl | acetyl | acetyl | H | H | acetyl | H | acetyl | acetyl |
| 2968 | acetyl | acetyl | acetyl | acetyl | H | H | acetyl | acetyl | H | H |
| 2969 | acetyl | acetyl | acetyl | acetyl | H | H | acetyl | acetyl | H | acetyl |
| 2970 | acetyl | acetyl | acetyl | acetyl | H | H | acetyl | acetyl | acetyl | H |
| 2971 | acetyl | acetyl | acetyl | acetyl | H | H | acetyl | acetyl | acetyl | acetyl |
| 2972 | acetyl | acetyl | acetyl | acetyl | H | acetyl | H | H | H | H |
| 2973 | acetyl | acetyl | acetyl | acetyl | H | acetyl | H | H | H | acetyl |
| 2974 | acetyl | acetyl | acetyl | acetyl | H | acetyl | H | H | acetyl | H |
| 2975 | acetyl | acetyl | acetyl | acetyl | H | acetyl | H | H | acetyl | acetyl |
| 2976 | acetyl | acetyl | acetyl | acetyl | H | acetyl | H | acetyl | H | H |
| 2977 | acetyl | acetyl | acetyl | acetyl | H | acetyl | H | acetyl | H | acetyl |
| 2978 | acetyl | acetyl | acetyl | acetyl | H | acetyl | H | acetyl | acetyl | H |
| 2979 | acetyl | acetyl | acetyl | acetyl | H | acetyl | H | acetyl | acetyl | acetyl |
| 2980 | acetyl | acetyl | acetyl | acetyl | H | acetyl | acetyl | H | H | H |
| 2981 | acetyl | acetyl | acetyl | acetyl | H | acetyl | acetyl | H | H | acetyl |
| 2982 | acetyl | acetyl | acetyl | acetyl | H | acetyl | acetyl | H | acetyl | H |
| 2983 | acetyl | acetyl | acetyl | acetyl | H | acetyl | acetyl | H | acetyl | acetyl |
| 2984 | acetyl | acetyl | acetyl | acetyl | H | acetyl | acetyl | acetyl | H | H |
| 2985 | acetyl | acetyl | acetyl | acetyl | H | acetyl | acetyl | acetyl | H | acetyl |
| 2986 | acetyl | acetyl | acetyl | acetyl | H | acetyl | acetyl | acetyl | acetyl | H |
| 2987 | acetyl | acetyl | acetyl | acetyl | H | acetyl | acetyl | acetyl | acetyl | acetyl |
| 2988 | acetyl | acetyl | acetyl | acetyl | acetyl | H | H | H | H | H |
| 2989 | acetyl | acetyl | acetyl | acetyl | acetyl | H | H | H | H | acetyl |
| 2990 | acetyl | acetyl | acetyl | acetyl | acetyl | H | H | H | acetyl | H |
| 2991 | acetyl | acetyl | acetyl | acetyl | acetyl | H | H | H | acetyl | acetyl |
| 2992 | acetyl | acetyl | acetyl | acetyl | acetyl | H | H | acetyl | H | H |
| 2993 | acetyl | acetyl | acetyl | acetyl | acetyl | H | H | acetyl | H | acetyl |
| 2994 | acetyl | acetyl | acetyl | acetyl | acetyl | H | H | acetyl | acetyl | H |
| 2995 | acetyl | acetyl | acetyl | acetyl | acetyl | H | H | acetyl | acetyl | acetyl |
| 2996 | acetyl | acetyl | acetyl | acetyl | acetyl | H | acetyl | H | H | H |
| 2997 | acetyl | acetyl | acetyl | acetyl | acetyl | H | acetyl | H | H | acetyl |
| 2998 | acetyl | acetyl | acetyl | acetyl | acetyl | H | acetyl | H | acetyl | H |
| 2999 | acetyl | acetyl | acetyl | acetyl | acetyl | H | acetyl | H | acetyl | acetyl |
| 3000 | acetyl | acetyl | acetyl | acetyl | acetyl | H | acetyl | acetyl | H | H |
| 3001 | acetyl | acetyl | acetyl | acetyl | acetyl | H | acetyl | acetyl | H | acetyl |
| 3002 | acetyl | acetyl | acetyl | acetyl | acetyl | H | acetyl | acetyl | acetyl | H |
| 3003 | acetyl | acetyl | acetyl | acetyl | acetyl | H | acetyl | acetyl | acetyl | acetyl |
| 3004 | acetyl | acetyl | acetyl | acetyl | acetyl | acetyl | H | H | H | H |
| 3005 | acetyl | acetyl | acetyl | acetyl | acetyl | acetyl | H | H | H | acetyl |
| 3006 | acetyl | acetyl | acetyl | acetyl | acetyl | acetyl | H | H | acetyl | H |
| 3007 | acetyl | acetyl | acetyl | acetyl | acetyl | acetyl | H | H | acetyl | acetyl |
| 3008 | acetyl | acetyl | acetyl | acetyl | acetyl | acetyl | H | acetyl | H | H |
| 3009 | acetyl | acetyl | acetyl | acetyl | acetyl | acetyl | H | acetyl | H | acetyl |
| 3010 | acetyl | acetyl | acetyl | acetyl | acetyl | acetyl | H | acetyl | acetyl | H |
| 3011 | acetyl | acetyl | acetyl | acetyl | acetyl | acetyl | H | acetyl | acetyl | acetyl |
| 3012 | acetyl | acetyl | acetyl | acetyl | acetyl | acetyl | acetyl | H | H | H |
| 3013 | acetyl | acetyl | acetyl | acetyl | acetyl | acetyl | acetyl | H | H | acetyl |
| 3014 | acetyl | acetyl | acetyl | acetyl | acetyl | acetyl | acetyl | H | acetyl | H |
| 3015 | acetyl | acetyl | acetyl | acetyl | acetyl | acetyl | acetyl | H | acetyl | acetyl |
| 3016 | acetyl | acetyl | acetyl | acetyl | acetyl | acetyl | acetyl | acetyl | H | H |
| 3017 | acetyl | acetyl | acetyl | acetyl | acetyl | acetyl | acetyl | acetyl | H | acetyl |
| 3018 | acetyl | acetyl | acetyl | acetyl | acetyl | acetyl | acetyl | acetyl | acetyl | H |

TABLE 8
(undecasaccharides) (Y is either H or a carrier)

| | | | | | | | | | | | n10; |
|---|---|---|---|---|---|---|---|---|---|---|---|
| Compound | R2 | n1, R1 | n2, R1 | n3, R1 | n4, R1 | n5, R1 | n6, R1 | n7, R1 | n8, R1 | n9, R1 | R1 |
| 3019 | H | H | H | H | H | H | H | H | H | H | acetyl |
| 3020 | H | H | H | H | H | H | H | H | H | acetyl | H |
| 3021 | H | H | H | H | H | H | H | H | H | acetyl | acetyl |
| 3022 | H | H | H | H | H | H | H | H | acetyl | H | H |
| 3023 | H | H | H | H | H | H | H | H | acetyl | H | acetyl |
| 3024 | H | H | H | H | H | H | H | H | acetyl | acetyl | H |
| 3025 | H | H | H | H | H | H | H | H | acetyl | acetyl | acetyl |
| 3026 | H | H | H | H | H | H | H | acetyl | H | H | H |
| 3027 | H | H | H | H | H | H | H | acetyl | H | H | acetyl |
| 3028 | H | H | H | H | H | H | H | acetyl | H | acetyl | H |
| 3029 | H | H | H | H | H | H | H | acetyl | H | acetyl | acetyl |
| 3030 | H | H | H | H | H | H | H | acetyl | acetyl | H | H |
| 3031 | H | H | H | H | H | H | H | acetyl | acetyl | H | acetyl |
| 3032 | H | H | H | H | H | H | H | acetyl | acetyl | acetyl | H |
| 3033 | H | H | H | H | H | H | H | acetyl | acetyl | acetyl | acetyl |
| 3034 | H | H | H | H | H | H | acetyl | H | H | H | H |
| 3035 | H | H | H | H | H | H | acetyl | H | H | H | acetyl |
| 3036 | H | H | H | H | H | H | acetyl | H | H | acetyl | H |
| 3037 | H | H | H | H | H | H | acetyl | H | H | acetyl | acetyl |
| 3038 | H | H | H | H | H | H | acetyl | H | acetyl | H | H |
| 3039 | H | H | H | H | H | H | acetyl | H | acetyl | H | acetyl |
| 3040 | H | H | H | H | H | H | acetyl | H | acetyl | acetyl | H |
| 3041 | H | H | H | H | H | H | acetyl | H | acetyl | acetyl | acetyl |
| 3042 | H | H | H | H | H | H | acetyl | acetyl | H | H | H |
| 3043 | H | H | H | H | H | H | acetyl | acetyl | H | H | acetyl |
| 3044 | H | H | H | H | H | H | acetyl | acetyl | H | acetyl | H |
| 3045 | H | H | H | H | H | H | acetyl | acetyl | H | acetyl | acetyl |
| 3046 | H | H | H | H | H | H | acetyl | acetyl | acetyl | H | H |
| 3047 | H | H | H | H | H | H | acetyl | acetyl | acetyl | H | acetyl |
| 3048 | H | H | H | H | H | H | acetyl | acetyl | acetyl | acetyl | H |
| 3049 | H | H | H | H | H | H | acetyl | acetyl | acetyl | acetyl | acetyl |
| 3050 | H | H | H | H | H | acetyl | H | H | H | H | H |
| 3051 | H | H | H | H | H | acetyl | H | H | H | H | acetyl |
| 3052 | H | H | H | H | H | acetyl | H | H | H | acetyl | H |
| 3053 | H | H | H | H | H | acetyl | H | H | H | acetyl | acetyl |
| 3054 | H | H | H | H | H | acetyl | H | H | acetyl | H | H |
| 3055 | H | H | H | H | H | acetyl | H | H | acetyl | H | acetyl |
| 3056 | H | H | H | H | H | acetyl | H | H | acetyl | acetyl | H |
| 3057 | H | H | H | H | H | acetyl | H | H | acetyl | acetyl | acetyl |
| 3058 | H | H | H | H | H | acetyl | H | acetyl | H | H | H |
| 3059 | H | H | H | H | H | acetyl | H | acetyl | H | H | acetyl |
| 3060 | H | H | H | H | H | acetyl | H | acetyl | H | acetyl | H |
| 3061 | H | H | H | H | H | acetyl | H | acetyl | H | acetyl | acetyl |
| 3062 | H | H | H | H | H | acetyl | H | acetyl | acetyl | H | H |
| 3063 | H | H | H | H | H | acetyl | H | acetyl | acetyl | H | acetyl |
| 3064 | H | H | H | H | H | acetyl | H | acetyl | acetyl | acetyl | H |
| 3065 | H | H | H | H | H | acetyl | H | acetyl | acetyl | acetyl | acetyl |
| 3066 | H | H | H | H | H | acetyl | acetyl | H | H | H | H |
| 3067 | H | H | H | H | H | acetyl | acetyl | H | H | H | acetyl |
| 3068 | H | H | H | H | H | acetyl | acetyl | H | H | acetyl | H |
| 3069 | H | H | H | H | H | acetyl | acetyl | H | H | acetyl | acetyl |
| 3070 | H | H | H | H | H | acetyl | acetyl | H | acetyl | H | H |
| 3071 | H | H | H | H | H | acetyl | acetyl | H | acetyl | H | acetyl |
| 3072 | H | H | H | H | H | acetyl | acetyl | H | acetyl | acetyl | H |
| 3073 | H | H | H | H | H | acetyl | acetyl | H | acetyl | acetyl | acetyl |
| 3074 | H | H | H | H | H | acetyl | acetyl | acetyl | H | H | H |
| 3075 | H | H | H | H | H | acetyl | acetyl | acetyl | H | H | acetyl |
| 3076 | H | H | H | H | H | acetyl | acetyl | acetyl | H | acetyl | H |
| 3077 | H | H | H | H | H | acetyl | acetyl | acetyl | H | acetyl | acetyl |
| 3078 | H | H | H | H | H | acetyl | acetyl | acetyl | acetyl | H | H |
| 3079 | H | H | H | H | H | acetyl | acetyl | acetyl | acetyl | H | acetyl |
| 3080 | H | H | H | H | H | acetyl | acetyl | acetyl | acetyl | acetyl | H |
| 3081 | H | H | H | H | H | acetyl | acetyl | acetyl | acetyl | acetyl | acetyl |
| 3082 | H | H | H | H | acetyl | H | H | H | H | H | H |
| 3083 | H | H | H | H | acetyl | H | H | H | H | H | acetyl |
| 3084 | H | H | H | H | acetyl | H | H | H | H | acetyl | H |
| 3085 | H | H | H | H | acetyl | H | H | H | H | acetyl | acetyl |
| 3086 | H | H | H | H | acetyl | H | H | H | acetyl | H | H |
| 3087 | H | H | H | H | acetyl | H | H | H | acetyl | H | acetyl |
| 3088 | H | H | H | H | acetyl | H | H | H | acetyl | acetyl | H |
| 3089 | H | H | H | H | acetyl | H | H | H | acetyl | acetyl | acetyl |
| 3090 | H | H | H | H | acetyl | H | H | acetyl | H | H | H |
| 3091 | H | H | H | H | acetyl | H | H | acetyl | H | H | acetyl |
| 3092 | H | H | H | H | acetyl | H | H | acetyl | H | acetyl | H |
| 3093 | H | H | H | H | acetyl | H | H | acetyl | H | acetyl | acetyl |
| 3094 | H | H | H | H | acetyl | H | H | acetyl | acetyl | H | H |
| 3095 | H | H | H | H | acetyl | H | H | acetyl | acetyl | H | acetyl |
| 3096 | H | H | H | H | acetyl | H | H | acetyl | acetyl | acetyl | H |
| 3097 | H | H | H | H | acetyl | H | H | acetyl | acetyl | acetyl | acetyl |
| 3098 | H | H | H | H | acetyl | H | acetyl | H | H | H | H |
| 3099 | H | H | H | H | acetyl | H | acetyl | H | H | H | acetyl |
| 3100 | H | H | H | H | acetyl | H | acetyl | H | H | acetyl | H |
| 3101 | H | H | H | H | acetyl | H | acetyl | H | H | acetyl | acetyl |
| 3102 | H | H | H | H | acetyl | H | acetyl | H | acetyl | H | H |
| 3103 | H | H | H | H | acetyl | H | acetyl | H | acetyl | H | acetyl |
| 3104 | H | H | H | H | acetyl | H | acetyl | H | acetyl | acetyl | H |
| 3105 | H | H | H | H | acetyl | H | acetyl | H | acetyl | acetyl | acetyl |
| 3106 | H | H | H | H | acetyl | H | acetyl | acetyl | H | H | H |
| 3107 | H | H | H | H | acetyl | H | acetyl | acetyl | H | H | acetyl |
| 3108 | H | H | H | H | acetyl | H | acetyl | acetyl | H | acetyl | H |
| 3109 | H | H | H | H | acetyl | H | acetyl | acetyl | H | acetyl | acetyl |
| 3110 | H | H | H | H | acetyl | H | acetyl | acetyl | acetyl | H | H |
| 3111 | H | H | H | H | acetyl | H | acetyl | acetyl | acetyl | H | acetyl |
| 3112 | H | H | H | H | acetyl | H | acetyl | acetyl | acetyl | acetyl | H |
| 3113 | H | H | H | H | acetyl | H | acetyl | acetyl | acetyl | acetyl | acetyl |
| 3114 | H | H | H | H | acetyl | acetyl | H | H | H | H | H |
| 3115 | H | H | H | H | acetyl | acetyl | H | H | H | H | acetyl |
| 3116 | H | H | H | H | acetyl | acetyl | H | H | H | acetyl | H |
| 3117 | H | H | H | H | acetyl | acetyl | H | H | H | acetyl | acetyl |
| 3118 | H | H | H | H | acetyl | acetyl | H | H | acetyl | H | H |
| 3119 | H | H | H | H | acetyl | acetyl | H | H | acetyl | H | acetyl |
| 3120 | H | H | H | H | acetyl | acetyl | H | H | acetyl | acetyl | H |
| 3121 | H | H | H | H | acetyl | acetyl | H | H | acetyl | acetyl | acetyl |
| 3122 | H | H | H | H | acetyl | acetyl | H | acetyl | H | H | H |
| 3123 | H | H | H | H | acetyl | acetyl | H | acetyl | H | H | acetyl |
| 3124 | H | H | H | H | acetyl | acetyl | H | acetyl | H | acetyl | H |
| 3125 | H | H | H | H | acetyl | acetyl | H | acetyl | H | acetyl | acetyl |
| 3126 | H | H | H | H | acetyl | acetyl | H | acetyl | acetyl | H | H |
| 3127 | H | H | H | H | acetyl | acetyl | H | acetyl | acetyl | H | acetyl |
| 3128 | H | H | H | H | acetyl | acetyl | H | acetyl | acetyl | acetyl | H |
| 3129 | H | H | H | H | acetyl | acetyl | H | acetyl | acetyl | acetyl | acetyl |
| 3130 | H | H | H | H | acetyl | acetyl | acetyl | H | H | H | H |
| 3131 | H | H | H | H | acetyl | acetyl | acetyl | H | H | H | acetyl |
| 3132 | H | H | H | H | acetyl | acetyl | acetyl | H | H | acetyl | H |
| 3133 | H | H | H | H | acetyl | acetyl | acetyl | H | H | acetyl | acetyl |
| 3134 | H | H | H | H | acetyl | acetyl | acetyl | H | acetyl | H | H |
| 3135 | H | H | H | H | acetyl | acetyl | acetyl | H | acetyl | H | acetyl |
| 3136 | H | H | H | H | acetyl | acetyl | acetyl | H | acetyl | acetyl | H |
| 3137 | H | H | H | H | acetyl | acetyl | acetyl | H | acetyl | acetyl | acetyl |
| 3138 | H | H | H | H | acetyl | acetyl | acetyl | acetyl | H | H | H |
| 3139 | H | H | H | H | acetyl | acetyl | acetyl | acetyl | H | H | acetyl |
| 3140 | H | H | H | H | acetyl | acetyl | acetyl | acetyl | H | acetyl | H |
| 3141 | H | H | H | H | acetyl | acetyl | acetyl | acetyl | H | acetyl | acetyl |
| 3142 | H | H | H | H | acetyl | acetyl | acetyl | acetyl | acetyl | H | H |
| 3143 | H | H | H | H | acetyl | acetyl | acetyl | acetyl | acetyl | H | acetyl |
| 3144 | H | H | H | H | acetyl | acetyl | acetyl | acetyl | acetyl | acetyl | H |
| 3145 | H | H | H | H | acetyl | acetyl | acetyl | acetyl | acetyl | acetyl | acetyl |
| 3146 | H | H | H | acetyl | H | H | H | H | H | H | H |
| 3147 | H | H | H | acetyl | H | H | H | H | H | H | acetyl |
| 3148 | H | H | H | acetyl | H | H | H | H | H | acetyl | H |
| 3149 | H | H | H | acetyl | H | H | H | H | H | acetyl | acetyl |
| 3150 | H | H | H | acetyl | H | H | H | H | acetyl | H | H |
| 3151 | H | H | H | acetyl | H | H | H | H | acetyl | H | acetyl |
| 3152 | H | H | H | acetyl | H | H | H | H | acetyl | acetyl | H |
| 3153 | H | H | H | acetyl | H | H | H | H | acetyl | acetyl | acetyl |
| 3154 | H | H | H | acetyl | H | H | H | acetyl | H | H | H |
| 3155 | H | H | H | acetyl | H | H | H | acetyl | H | H | acetyl |
| 3156 | H | H | H | acetyl | H | H | H | acetyl | H | acetyl | H |
| 3157 | H | H | H | acetyl | H | H | H | acetyl | H | acetyl | acetyl |
| 3158 | H | H | H | acetyl | H | H | H | acetyl | acetyl | H | H |
| 3159 | H | H | H | acetyl | H | H | H | acetyl | acetyl | H | acetyl |
| 3160 | H | H | H | acetyl | H | H | H | acetyl | acetyl | acetyl | H |
| 3161 | H | H | H | acetyl | H | H | H | acetyl | acetyl | acetyl | acetyl |
| 3162 | H | H | H | acetyl | H | H | acetyl | H | H | H | H |
| 3163 | H | H | H | acetyl | H | H | acetyl | H | H | H | acetyl |
| 3164 | H | H | H | acetyl | H | H | acetyl | H | H | acetyl | H |
| 3165 | H | H | H | acetyl | H | H | acetyl | H | H | acetyl | acetyl |
| 3166 | H | H | H | acetyl | H | H | acetyl | H | acetyl | H | H |
| 3167 | H | H | H | acetyl | H | H | acetyl | H | acetyl | H | acetyl |
| 3168 | H | H | H | acetyl | H | H | acetyl | H | acetyl | acetyl | H |
| 3169 | H | H | H | acetyl | H | H | acetyl | H | acetyl | acetyl | acetyl |
| 3170 | H | H | H | acetyl | H | H | acetyl | acetyl | H | H | H |
| 3171 | H | H | H | acetyl | H | H | acetyl | acetyl | H | H | acetyl |
| 3172 | H | H | H | acetyl | H | H | acetyl | acetyl | H | acetyl | H |
| 3173 | H | H | H | acetyl | H | H | acetyl | acetyl | H | acetyl | acetyl |
| 3174 | H | H | H | acetyl | H | H | acetyl | acetyl | acetyl | H | H |
| 3175 | H | H | H | acetyl | H | H | acetyl | acetyl | acetyl | H | acetyl |
| 3176 | H | H | H | acetyl | H | H | acetyl | acetyl | acetyl | acetyl | H |
| 3177 | H | H | H | acetyl | H | H | acetyl | acetyl | acetyl | acetyl | acetyl |
| 3178 | H | H | H | acetyl | H | acetyl | H | H | H | H | H |
| 3179 | H | H | H | acetyl | H | acetyl | H | H | H | H | acetyl |
| 3180 | H | H | H | acetyl | H | acetyl | H | H | H | acetyl | H |
| 3181 | H | H | H | acetyl | H | acetyl | H | H | H | acetyl | acetyl |
| 3182 | H | H | H | acetyl | H | acetyl | H | H | acetyl | H | H |
| 3183 | H | H | H | acetyl | H | acetyl | H | H | acetyl | H | acetyl |
| 3184 | H | H | H | acetyl | H | acetyl | H | H | acetyl | acetyl | H |
| 3185 | H | H | H | acetyl | H | acetyl | H | H | acetyl | acetyl | acetyl |
| 3186 | H | H | H | acetyl | H | acetyl | H | acetyl | H | H | H |
| 3187 | H | H | H | acetyl | H | acetyl | H | acetyl | H | H | acetyl |
| 3188 | H | H | H | acetyl | H | acetyl | H | acetyl | H | acetyl | H |
| 3189 | H | H | H | acetyl | H | acetyl | H | acetyl | H | acetyl | acetyl |
| 3190 | H | H | H | acetyl | H | acetyl | H | acetyl | acetyl | H | H |
| 3191 | H | H | H | acetyl | H | acetyl | H | acetyl | acetyl | H | acetyl |
| 3192 | H | H | H | acetyl | H | acetyl | H | acetyl | acetyl | acetyl | H |
| 3193 | H | H | H | acetyl | H | acetyl | H | acetyl | acetyl | acetyl | acetyl |
| 3194 | H | H | H | acetyl | H | acetyl | acetyl | H | H | H | H |
| 3195 | H | H | H | acetyl | H | acetyl | acetyl | H | H | H | acetyl |
| 3196 | H | H | H | acetyl | H | acetyl | acetyl | H | H | acetyl | H |
| 3197 | H | H | H | acetyl | H | acetyl | acetyl | H | H | acetyl | acetyl |
| 3198 | H | H | H | acetyl | H | acetyl | acetyl | H | acetyl | H | H |
| 3199 | H | H | H | acetyl | H | acetyl | acetyl | H | acetyl | H | acetyl |
| 3200 | H | H | H | acetyl | H | acetyl | acetyl | H | acetyl | acetyl | H |
| 3201 | H | H | H | acetyl | H | acetyl | acetyl | H | acetyl | acetyl | acetyl |
| 3202 | H | H | H | acetyl | H | acetyl | acetyl | acetyl | H | H | H |
| 3203 | H | H | H | acetyl | H | acetyl | acetyl | acetyl | H | H | acetyl |
| 3204 | H | H | H | acetyl | H | acetyl | acetyl | acetyl | H | acetyl | H |
| 3205 | H | H | H | acetyl | H | acetyl | acetyl | acetyl | H | acetyl | acetyl |
| 3206 | H | H | H | acetyl | H | acetyl | acetyl | acetyl | acetyl | H | H |
| 3207 | H | H | H | acetyl | H | acetyl | acetyl | acetyl | acetyl | H | acetyl |
| 3208 | H | H | H | acetyl | H | acetyl | acetyl | acetyl | acetyl | acetyl | H |
| 3209 | H | H | H | acetyl | H | acetyl | acetyl | acetyl | acetyl | acetyl | acetyl |
| 3210 | H | H | H | acetyl | acetyl | H | H | H | H | H | H |
| 3211 | H | H | H | acetyl | acetyl | H | H | H | H | H | acetyl |
| 3212 | H | H | H | acetyl | acetyl | H | H | H | H | acetyl | H |
| 3213 | H | H | H | acetyl | acetyl | H | H | H | H | acetyl | acetyl |
| 3214 | H | H | H | acetyl | acetyl | H | H | H | acetyl | H | H |
| 3215 | H | H | H | acetyl | acetyl | H | H | H | acetyl | H | acetyl |
| 3216 | H | H | H | acetyl | acetyl | H | H | H | acetyl | acetyl | H |
| 3217 | H | H | H | acetyl | acetyl | H | H | H | acetyl | acetyl | acetyl |
| 3218 | H | H | H | acetyl | acetyl | H | H | acetyl | H | H | H |
| 3219 | H | H | H | acetyl | acetyl | H | H | acetyl | H | H | acetyl |
| 3220 | H | H | H | acetyl | acetyl | H | H | acetyl | H | acetyl | H |
| 3221 | H | H | H | acetyl | acetyl | H | H | acetyl | H | acetyl | acetyl |
| 3222 | H | H | H | acetyl | acetyl | H | H | acetyl | acetyl | H | H |
| 3223 | H | H | H | acetyl | acetyl | H | H | acetyl | acetyl | H | acetyl |
| 3224 | H | H | H | acetyl | acetyl | H | H | acetyl | acetyl | acetyl | H |
| 3225 | H | H | H | acetyl | acetyl | H | H | acetyl | acetyl | acetyl | acetyl |
| 3226 | H | H | H | acetyl | acetyl | H | acetyl | H | H | H | H |
| 3227 | H | H | H | acetyl | acetyl | H | acetyl | H | H | H | acetyl |
| 3228 | H | H | H | acetyl | acetyl | H | acetyl | H | H | acetyl | H |
| 3229 | H | H | H | acetyl | acetyl | H | acetyl | H | H | acetyl | acetyl |
| 3230 | H | H | H | acetyl | acetyl | H | acetyl | H | acetyl | H | H |
| 3231 | H | H | H | acetyl | acetyl | H | acetyl | H | acetyl | H | acetyl |
| 3232 | H | H | H | acetyl | acetyl | H | acetyl | H | acetyl | acetyl | H |
| 3233 | H | H | H | acetyl | acetyl | H | acetyl | H | acetyl | acetyl | acetyl |
| 3234 | H | H | H | acetyl | acetyl | H | acetyl | acetyl | H | H | H |
| 3235 | H | H | H | acetyl | acetyl | H | acetyl | acetyl | H | H | acetyl |
| 3236 | H | H | H | acetyl | acetyl | H | acetyl | acetyl | H | acetyl | H |
| 3237 | H | H | H | acetyl | acetyl | H | acetyl | acetyl | H | acetyl | acetyl |
| 3238 | H | H | H | acetyl | acetyl | H | acetyl | acetyl | acetyl | H | H |
| 3239 | H | H | H | acetyl | acetyl | H | acetyl | acetyl | acetyl | H | acetyl |
| 3240 | H | H | H | acetyl | acetyl | H | acetyl | acetyl | acetyl | acetyl | H |
| 3241 | H | H | H | acetyl | acetyl | H | acetyl | acetyl | acetyl | acetyl | acetyl |
| 3242 | H | H | H | acetyl | acetyl | acetyl | H | H | H | H | H |
| 3243 | H | H | H | acetyl | acetyl | acetyl | H | H | H | H | acetyl |
| 3244 | H | H | H | acetyl | acetyl | acetyl | H | H | H | acetyl | H |
| 3245 | H | H | H | acetyl | acetyl | acetyl | H | H | H | acetyl | acetyl |
| 3246 | H | H | H | acetyl | acetyl | acetyl | H | H | acetyl | H | H |
| 3247 | H | H | H | acetyl | acetyl | acetyl | H | H | acetyl | H | acetyl |
| 3248 | H | H | H | acetyl | acetyl | acetyl | H | H | acetyl | acetyl | H |
| 3249 | H | H | H | acetyl | acetyl | acetyl | H | H | acetyl | acetyl | acetyl |
| 3250 | H | H | H | acetyl | acetyl | acetyl | H | acetyl | H | H | H |
| 3251 | H | H | H | acetyl | acetyl | acetyl | H | acetyl | H | H | acetyl |
| 3252 | H | H | H | acetyl | acetyl | acetyl | H | acetyl | H | acetyl | H |
| 3253 | H | H | H | acetyl | acetyl | acetyl | H | acetyl | H | acetyl | acetyl |
| 3254 | H | H | H | acetyl | acetyl | acetyl | H | acetyl | acetyl | H | H |
| 3255 | H | H | H | acetyl | acetyl | acetyl | H | acetyl | acetyl | H | acetyl |
| 3256 | H | H | H | acetyl | acetyl | acetyl | H | acetyl | acetyl | acetyl | H |
| 3257 | H | H | H | acetyl | acetyl | acetyl | H | acetyl | acetyl | acetyl | acetyl |
| 3258 | H | H | H | acetyl | acetyl | acetyl | acetyl | H | H | H | H |
| 3259 | H | H | H | acetyl | acetyl | acetyl | acetyl | H | H | H | acetyl |
| 3260 | H | H | H | acetyl | acetyl | acetyl | acetyl | H | H | acetyl | H |
| 3261 | H | H | H | acetyl | acetyl | acetyl | acetyl | H | H | acetyl | acetyl |
| 3262 | H | H | H | acetyl | acetyl | acetyl | acetyl | H | acetyl | H | H |
| 3263 | H | H | H | acetyl | acetyl | acetyl | acetyl | H | acetyl | H | acetyl |
| 3264 | H | H | H | acetyl | acetyl | acetyl | acetyl | H | acetyl | acetyl | H |
| 3265 | H | H | H | acetyl | acetyl | acetyl | acetyl | H | acetyl | acetyl | acetyl |
| 3266 | H | H | H | acetyl | acetyl | acetyl | acetyl | acetyl | H | H | H |
| 3267 | H | H | H | acetyl | acetyl | acetyl | acetyl | acetyl | H | H | acetyl |
| 3268 | H | H | H | acetyl | acetyl | acetyl | acetyl | acetyl | H | acetyl | H |
| 3269 | H | H | H | acetyl | acetyl | acetyl | acetyl | acetyl | H | acetyl | acetyl |
| 3270 | H | H | H | acetyl | acetyl | acetyl | acetyl | acetyl | acetyl | H | H |
| 3271 | H | H | H | acetyl | acetyl | acetyl | acetyl | acetyl | acetyl | H | acetyl |
| 3272 | H | H | H | acetyl | acetyl | acetyl | acetyl | acetyl | acetyl | acetyl | H |
| 3273 | H | H | H | acetyl | acetyl | acetyl | acetyl | acetyl | acetyl | acetyl | acetyl |
| 3274 | H | H | acetyl | H | H | H | H | H | H | H | H |
| 3275 | H | H | acetyl | H | H | H | H | H | H | H | acetyl |
| 3276 | H | H | acetyl | H | H | H | H | H | H | acetyl | H |
| 3277 | H | H | acetyl | H | H | H | H | H | H | acetyl | acetyl |
| 3278 | H | H | acetyl | H | H | H | H | H | acetyl | H | H |
| 3279 | H | H | acetyl | H | H | H | H | H | acetyl | H | acetyl |
| 3280 | H | H | acetyl | H | H | H | H | H | acetyl | acetyl | H |
| 3281 | H | H | acetyl | H | H | H | H | H | acetyl | acetyl | acetyl |
| 3282 | H | H | acetyl | H | H | H | H | acetyl | H | H | H |
| 3283 | H | H | acetyl | H | H | H | H | acetyl | H | H | acetyl |
| 3284 | H | H | acetyl | H | H | H | H | acetyl | H | acetyl | H |
| 3285 | H | H | acetyl | H | H | H | H | acetyl | H | acetyl | acetyl |
| 3286 | H | H | acetyl | H | H | H | H | acetyl | acetyl | H | H |
| 3287 | H | H | acetyl | H | H | H | H | acetyl | acetyl | H | acetyl |
| 3288 | H | H | acetyl | H | H | H | H | acetyl | acetyl | acetyl | H |
| 3289 | H | H | acetyl | H | H | H | H | acetyl | acetyl | acetyl | acetyl |
| 3290 | H | H | acetyl | H | H | H | acetyl | H | H | H | H |
| 3291 | H | H | acetyl | H | H | H | acetyl | H | H | H | acetyl |
| 3292 | H | H | acetyl | H | H | H | acetyl | H | H | acetyl | H |
| 3293 | H | H | acetyl | H | H | H | acetyl | H | H | acetyl | acetyl |
| 3294 | H | H | acetyl | H | H | H | acetyl | H | acetyl | H | H |
| 3295 | H | H | acetyl | H | H | H | acetyl | H | acetyl | H | acetyl |
| 3296 | H | H | acetyl | H | H | H | acetyl | H | acetyl | acetyl | H |
| 3297 | H | H | acetyl | H | H | H | acetyl | H | acetyl | acetyl | acetyl |
| 3298 | H | H | acetyl | H | H | H | acetyl | acetyl | H | H | H |
| 3299 | H | H | acetyl | H | H | H | acetyl | acetyl | H | H | acetyl |
| 3300 | H | H | acetyl | H | H | H | acetyl | acetyl | H | acetyl | H |
| 3301 | H | H | acetyl | H | H | H | acetyl | acetyl | H | acetyl | acetyl |
| 3302 | H | H | acetyl | H | H | H | acetyl | acetyl | acetyl | H | H |
| 3303 | H | H | acetyl | H | H | H | acetyl | acetyl | acetyl | H | acetyl |
| 3304 | H | H | acetyl | H | H | H | acetyl | acetyl | acetyl | acetyl | H |
| 3305 | H | H | acetyl | H | H | H | acetyl | acetyl | acetyl | acetyl | acetyl |
| 3306 | H | H | acetyl | H | H | acetyl | H | H | H | H | H |
| 3307 | H | H | acetyl | H | H | acetyl | H | H | H | H | acetyl |
| 3308 | H | H | acetyl | H | H | acetyl | H | H | H | acetyl | H |
| 3309 | H | H | acetyl | H | H | acetyl | H | H | H | acetyl | acetyl |
| 3310 | H | H | acetyl | H | H | acetyl | H | H | acetyl | H | H |
| 3311 | H | H | acetyl | H | H | acetyl | H | H | acetyl | H | acetyl |
| 3312 | H | H | acetyl | H | H | acetyl | H | H | acetyl | acetyl | H |
| 3313 | H | H | acetyl | H | H | acetyl | H | H | acetyl | acetyl | acetyl |
| 3314 | H | H | acetyl | H | H | acetyl | H | acetyl | H | H | H |
| 3315 | H | H | acetyl | H | H | acetyl | H | acetyl | H | H | acetyl |
| 3316 | H | H | acetyl | H | H | acetyl | H | acetyl | H | acetyl | H |
| 3317 | H | H | acetyl | H | H | acetyl | H | acetyl | H | acetyl | acetyl |
| 3318 | H | H | acetyl | H | H | acetyl | H | acetyl | acetyl | H | H |
| 3319 | H | H | acetyl | H | H | acetyl | H | acetyl | acetyl | H | acetyl |
| 3320 | H | H | acetyl | H | H | acetyl | H | acetyl | acetyl | acetyl | H |
| 3321 | H | H | acetyl | H | H | acetyl | H | acetyl | acetyl | acetyl | acetyl |
| 3322 | H | H | acetyl | H | H | acetyl | acetyl | H | H | H | H |
| 3323 | H | H | acetyl | H | H | acetyl | acetyl | H | H | H | acetyl |
| 3324 | H | H | acetyl | H | H | acetyl | acetyl | H | H | acetyl | H |
| 3325 | H | H | acetyl | H | H | acetyl | acetyl | H | H | acetyl | acetyl |
| 3326 | H | H | acetyl | H | H | acetyl | acetyl | H | acetyl | H | H |
| 3327 | H | H | acetyl | H | H | acetyl | acetyl | H | acetyl | H | acetyl |
| 3328 | H | H | acetyl | H | H | acetyl | acetyl | H | acetyl | acetyl | H |
| 3329 | H | H | acetyl | H | H | acetyl | acetyl | H | acetyl | acetyl | acetyl |
| 3330 | H | H | acetyl | H | H | acetyl | acetyl | acetyl | H | H | H |
| 3331 | H | H | acetyl | H | H | acetyl | acetyl | acetyl | H | H | acetyl |
| 3332 | H | H | acetyl | H | H | acetyl | acetyl | acetyl | H | acetyl | H |
| 3333 | H | H | acetyl | H | H | acetyl | acetyl | acetyl | H | acetyl | acetyl |
| 3334 | H | H | acetyl | H | H | acetyl | acetyl | acetyl | acetyl | H | H |
| 3335 | H | H | acetyl | H | H | acetyl | acetyl | acetyl | acetyl | H | acetyl |
| 3336 | H | H | acetyl | H | H | acetyl | acetyl | acetyl | acetyl | acetyl | H |
| 3337 | H | H | acetyl | H | H | acetyl | acetyl | acetyl | acetyl | acetyl | acetyl |
| 3338 | H | H | acetyl | H | acetyl | H | H | H | H | H | H |
| 3339 | H | H | acetyl | H | acetyl | H | H | H | H | H | acetyl |
| 3340 | H | H | acetyl | H | acetyl | H | H | H | H | acetyl | H |
| 3341 | H | H | acetyl | H | acetyl | H | H | H | H | acetyl | acetyl |
| 3342 | H | H | acetyl | H | acetyl | H | H | H | acetyl | H | H |
| 3343 | H | H | acetyl | H | acetyl | H | H | H | acetyl | H | acetyl |
| 3344 | H | H | acetyl | H | acetyl | H | H | H | acetyl | acetyl | H |
| 3345 | H | H | acetyl | H | acetyl | H | H | H | acetyl | acetyl | acetyl |
| 3346 | H | H | acetyl | H | acetyl | H | H | acetyl | H | H | H |
| 3347 | H | H | acetyl | H | acetyl | H | H | acetyl | H | H | acetyl |
| 3348 | H | H | acetyl | H | acetyl | H | H | acetyl | H | acetyl | H |
| 3349 | H | H | acetyl | H | acetyl | H | H | acetyl | H | acetyl | acetyl |
| 3350 | H | H | acetyl | H | acetyl | H | H | acetyl | acetyl | H | H |
| 3351 | H | H | acetyl | H | acetyl | H | H | acetyl | acetyl | H | acetyl |
| 3352 | H | H | acetyl | H | acetyl | H | H | acetyl | acetyl | acetyl | H |
| 3353 | H | H | acetyl | H | acetyl | H | H | acetyl | acetyl | acetyl | acetyl |
| 3354 | H | H | acetyl | H | acetyl | H | acetyl | H | H | H | H |
| 3355 | H | H | acetyl | H | acetyl | H | acetyl | H | H | H | acetyl |
| 3356 | H | H | acetyl | H | acetyl | H | acetyl | H | H | acetyl | H |
| 3357 | H | H | acetyl | H | acetyl | H | acetyl | H | H | acetyl | acetyl |
| 3358 | H | H | acetyl | H | acetyl | H | acetyl | H | acetyl | H | H |
| 3359 | H | H | acetyl | H | acetyl | H | acetyl | H | acetyl | H | acetyl |
| 3360 | H | H | acetyl | H | acetyl | H | acetyl | H | acetyl | acetyl | H |
| 3361 | H | H | acetyl | H | acetyl | H | acetyl | H | acetyl | acetyl | acetyl |
| 3362 | H | H | acetyl | H | acetyl | H | acetyl | acetyl | H | H | H |
| 3363 | H | H | acetyl | H | acetyl | H | acetyl | acetyl | H | H | acetyl |
| 3364 | H | H | acetyl | H | acetyl | H | acetyl | acetyl | H | acetyl | H |
| 3365 | H | H | acetyl | H | acetyl | H | acetyl | acetyl | H | acetyl | acetyl |
| 3366 | H | H | acetyl | H | acetyl | H | acetyl | acetyl | acetyl | H | H |
| 3367 | H | H | acetyl | H | acetyl | H | acetyl | acetyl | acetyl | H | acetyl |
| 3368 | H | H | acetyl | H | acetyl | H | acetyl | acetyl | acetyl | acetyl | H |
| 3369 | H | H | acetyl | H | acetyl | H | acetyl | acetyl | acetyl | acetyl | acetyl |
| 3370 | H | H | acetyl | H | acetyl | acetyl | H | H | H | H | H |
| 3371 | H | H | acetyl | H | acetyl | acetyl | H | H | H | H | acetyl |
| 3372 | H | H | acetyl | H | acetyl | acetyl | H | H | H | acetyl | H |
| 3373 | H | H | acetyl | H | acetyl | acetyl | H | H | H | acetyl | acetyl |
| 3374 | H | H | acetyl | H | acetyl | acetyl | H | H | acetyl | H | H |
| 3375 | H | H | acetyl | H | acetyl | acetyl | H | H | acetyl | H | acetyl |
| 3376 | H | H | acetyl | H | acetyl | acetyl | H | H | acetyl | acetyl | H |
| 3377 | H | H | acetyl | H | acetyl | acetyl | H | H | acetyl | acetyl | acetyl |
| 3378 | H | H | acetyl | H | acetyl | acetyl | H | acetyl | H | H | H |
| 3379 | H | H | acetyl | H | acetyl | acetyl | H | acetyl | H | H | acetyl |
| 3380 | H | H | acetyl | H | acetyl | acetyl | H | acetyl | H | acetyl | H |
| 3381 | H | H | acetyl | H | acetyl | acetyl | H | acetyl | H | acetyl | acetyl |
| 3382 | H | H | acetyl | H | acetyl | acetyl | H | acetyl | acetyl | H | H |
| 3383 | H | H | acetyl | H | acetyl | acetyl | H | acetyl | acetyl | H | acetyl |
| 3384 | H | H | acetyl | H | acetyl | acetyl | H | acetyl | acetyl | acetyl | H |
| 3385 | H | H | acetyl | H | acetyl | acetyl | H | acetyl | acetyl | acetyl | acetyl |
| 3386 | H | H | acetyl | H | acetyl | acetyl | acetyl | H | H | H | H |
| 3387 | H | H | acetyl | H | acetyl | acetyl | acetyl | H | H | H | acetyl |
| 3388 | H | H | acetyl | H | acetyl | acetyl | acetyl | H | H | acetyl | H |
| 3389 | H | H | acetyl | H | acetyl | acetyl | acetyl | H | H | acetyl | acetyl |
| 3390 | H | H | acetyl | H | acetyl | acetyl | acetyl | H | acetyl | H | H |
| 3391 | H | H | acetyl | H | acetyl | acetyl | acetyl | H | acetyl | H | acetyl |
| 3392 | H | H | acetyl | H | acetyl | acetyl | acetyl | H | acetyl | acetyl | H |
| 3393 | H | H | acetyl | H | acetyl | acetyl | acetyl | H | acetyl | acetyl | acetyl |
| 3394 | H | H | acetyl | H | acetyl | acetyl | acetyl | acetyl | H | H | H |
| 3395 | H | H | acetyl | H | acetyl | acetyl | acetyl | acetyl | H | H | acetyl |
| 3396 | H | H | acetyl | H | acetyl | acetyl | acetyl | acetyl | H | acetyl | H |
| 3397 | H | H | acetyl | H | acetyl | acetyl | acetyl | acetyl | H | acetyl | acetyl |
| 3398 | H | H | acetyl | H | acetyl | acetyl | acetyl | acetyl | acetyl | H | H |
| 3399 | H | H | acetyl | H | acetyl | acetyl | acetyl | acetyl | acetyl | H | acetyl |
| 3400 | H | H | acetyl | H | acetyl | acetyl | acetyl | acetyl | acetyl | acetyl | H |
| 3401 | H | H | acetyl | H | acetyl | acetyl | acetyl | acetyl | acetyl | acetyl | acetyl |
| 3402 | H | H | acetyl | acetyl | H | H | H | H | H | H | H |
| 3403 | H | H | acetyl | acetyl | H | H | H | H | H | H | acetyl |
| 3404 | H | H | acetyl | acetyl | H | H | H | H | H | acetyl | H |
| 3405 | H | H | acetyl | acetyl | H | H | H | H | H | acetyl | acetyl |
| 3406 | H | H | acetyl | acetyl | H | H | H | H | acetyl | H | H |
| 3407 | H | H | acetyl | acetyl | H | H | H | H | acetyl | H | acetyl |
| 3408 | H | H | acetyl | acetyl | H | H | H | H | acetyl | acetyl | H |
| 3409 | H | H | acetyl | acetyl | H | H | H | H | acetyl | acetyl | acetyl |
| 3410 | H | H | acetyl | acetyl | H | H | H | acetyl | H | H | H |
| 3411 | H | H | acetyl | acetyl | H | H | H | acetyl | H | H | acetyl |
| 3412 | H | H | acetyl | acetyl | H | H | H | acetyl | H | acetyl | H |
| 3413 | H | H | acetyl | acetyl | H | H | H | acetyl | H | acetyl | acetyl |
| 3414 | H | H | acetyl | acetyl | H | H | H | acetyl | acetyl | H | H |
| 3415 | H | H | acetyl | acetyl | H | H | H | acetyl | acetyl | H | acetyl |
| 3416 | H | H | acetyl | acetyl | H | H | H | acetyl | acetyl | acetyl | H |
| 3417 | H | H | acetyl | acetyl | H | H | H | acetyl | acetyl | acetyl | acetyl |
| 3418 | H | H | acetyl | acetyl | H | H | acetyl | H | H | H | H |
| 3419 | H | H | acetyl | acetyl | H | H | acetyl | H | H | H | acetyl |
| 3420 | H | H | acetyl | acetyl | H | H | acetyl | H | H | acetyl | H |
| 3421 | H | H | acetyl | acetyl | H | H | acetyl | H | H | acetyl | acetyl |
| 3422 | H | H | acetyl | acetyl | H | H | acetyl | H | acetyl | H | H |
| 3423 | H | H | acetyl | acetyl | H | H | acetyl | H | acetyl | H | acetyl |
| 3424 | H | H | acetyl | acetyl | H | H | acetyl | H | acetyl | acetyl | H |
| 3425 | H | H | acetyl | acetyl | H | H | acetyl | H | acetyl | acetyl | acetyl |
| 3426 | H | H | acetyl | acetyl | H | H | acetyl | acetyl | H | H | H |
| 3427 | H | H | acetyl | acetyl | H | H | acetyl | acetyl | H | H | acetyl |
| 3428 | H | H | acetyl | acetyl | H | H | acetyl | acetyl | H | acetyl | H |
| 3429 | H | H | acetyl | acetyl | H | H | acetyl | acetyl | H | acetyl | acetyl |
| 3430 | H | H | acetyl | acetyl | H | H | acetyl | acetyl | acetyl | H | H |
| 3431 | H | H | acetyl | acetyl | H | H | acetyl | acetyl | acetyl | H | acetyl |
| 3432 | H | H | acetyl | acetyl | H | H | acetyl | acetyl | acetyl | acetyl | H |
| 3433 | H | H | acetyl | acetyl | H | H | acetyl | acetyl | acetyl | acetyl | acetyl |
| 3434 | H | H | acetyl | acetyl | H | acetyl | H | H | H | H | H |
| 3435 | H | H | acetyl | acetyl | H | acetyl | H | H | H | H | acetyl |
| 3436 | H | H | acetyl | acetyl | H | acetyl | H | H | H | acetyl | H |
| 3437 | H | H | acetyl | acetyl | H | acetyl | H | H | H | acetyl | acetyl |
| 3438 | H | H | acetyl | acetyl | H | acetyl | H | H | acetyl | H | H |
| 3439 | H | H | acetyl | acetyl | H | acetyl | H | H | acetyl | H | acetyl |
| 3440 | H | H | acetyl | acetyl | H | acetyl | H | H | acetyl | acetyl | H |
| 3441 | H | H | acetyl | acetyl | H | acetyl | H | H | acetyl | acetyl | acetyl |
| 3442 | H | H | acetyl | acetyl | H | acetyl | H | acetyl | H | H | H |
| 3443 | H | H | acetyl | acetyl | H | acetyl | H | acetyl | H | H | acetyl |
| 3444 | H | H | acetyl | acetyl | H | acetyl | H | acetyl | H | acetyl | H |
| 3445 | H | H | acetyl | acetyl | H | acetyl | H | acetyl | H | acetyl | acetyl |
| 3446 | H | H | acetyl | acetyl | H | acetyl | H | acetyl | acetyl | H | H |
| 3447 | H | H | acetyl | acetyl | H | acetyl | H | acetyl | acetyl | H | acetyl |
| 3448 | H | H | acetyl | acetyl | H | acetyl | H | acetyl | acetyl | acetyl | H |
| 3449 | H | H | acetyl | acetyl | H | acetyl | H | acetyl | acetyl | acetyl | acetyl |
| 3450 | H | H | acetyl | acetyl | H | acetyl | acetyl | H | H | H | H |
| 3451 | H | H | acetyl | acetyl | H | acetyl | acetyl | H | H | H | acetyl |
| 3452 | H | H | acetyl | acetyl | H | acetyl | acetyl | H | H | acetyl | H |
| 3453 | H | H | acetyl | acetyl | H | acetyl | acetyl | H | H | acetyl | acetyl |
| 3454 | H | H | acetyl | acetyl | H | acetyl | acetyl | H | acetyl | H | H |
| 3455 | H | H | acetyl | acetyl | H | acetyl | acetyl | H | acetyl | H | acetyl |
| 3456 | H | H | acetyl | acetyl | H | acetyl | acetyl | H | acetyl | acetyl | H |
| 3457 | H | H | acetyl | acetyl | H | acetyl | acetyl | H | acetyl | acetyl | acetyl |
| 3458 | H | H | acetyl | acetyl | H | acetyl | acetyl | acetyl | H | H | H |
| 3459 | H | H | acetyl | acetyl | H | acetyl | acetyl | acetyl | H | H | acetyl |
| 3460 | H | H | acetyl | acetyl | H | acetyl | acetyl | acetyl | H | acetyl | H |
| 3461 | H | H | acetyl | acetyl | H | acetyl | acetyl | acetyl | H | acetyl | acetyl |
| 3462 | H | H | acetyl | acetyl | H | acetyl | acetyl | acetyl | acetyl | H | H |
| 3463 | H | H | acetyl | acetyl | H | acetyl | acetyl | acetyl | acetyl | H | acetyl |
| 3464 | H | H | acetyl | acetyl | H | acetyl | acetyl | acetyl | acetyl | acetyl | H |
| 3465 | H | H | acetyl | acetyl | H | acetyl | acetyl | acetyl | acetyl | acetyl | acetyl |
| 3466 | H | H | acetyl | acetyl | acetyl | H | H | H | H | H | H |
| 3467 | H | H | acetyl | acetyl | acetyl | H | H | H | H | H | acetyl |
| 3468 | H | H | acetyl | acetyl | acetyl | H | H | H | H | acetyl | H |
| 3469 | H | H | acetyl | acetyl | acetyl | H | H | H | H | acetyl | acetyl |
| 3470 | H | H | acetyl | acetyl | acetyl | H | H | H | acetyl | H | H |
| 3471 | H | H | acetyl | acetyl | acetyl | H | H | H | acetyl | H | acetyl |
| 3472 | H | H | acetyl | acetyl | acetyl | H | H | H | acetyl | acetyl | H |
| 3473 | H | H | acetyl | acetyl | acetyl | H | H | H | acetyl | acetyl | acetyl |
| 3474 | H | H | acetyl | acetyl | acetyl | H | H | acetyl | H | H | H |
| 3475 | H | H | acetyl | acetyl | acetyl | H | H | acetyl | H | H | acetyl |
| 3476 | H | H | acetyl | acetyl | acetyl | H | H | acetyl | H | acetyl | H |
| 3477 | H | H | acetyl | acetyl | acetyl | H | H | acetyl | H | acetyl | acetyl |
| 3478 | H | H | acetyl | acetyl | acetyl | H | H | acetyl | acetyl | H | H |
| 3479 | H | H | acetyl | acetyl | acetyl | H | H | acetyl | acetyl | H | acetyl |
| 3480 | H | H | acetyl | acetyl | acetyl | H | H | acetyl | acetyl | acetyl | H |
| 3481 | H | H | acetyl | acetyl | acetyl | H | H | acetyl | acetyl | acetyl | acetyl |
| 3482 | H | H | acetyl | acetyl | acetyl | H | acetyl | H | H | H | H |
| 3483 | H | H | acetyl | acetyl | acetyl | H | acetyl | H | H | H | acetyl |
| 3484 | H | H | acetyl | acetyl | acetyl | H | acetyl | H | H | acetyl | H |
| 3485 | H | H | acetyl | acetyl | acetyl | H | acetyl | H | H | acetyl | acetyl |
| 3486 | H | H | acetyl | acetyl | acetyl | H | acetyl | H | acetyl | H | H |
| 3487 | H | H | acetyl | acetyl | acetyl | H | acetyl | H | acetyl | H | acetyl |
| 3488 | H | H | acetyl | acetyl | acetyl | H | acetyl | H | acetyl | acetyl | H |
| 3489 | H | H | acetyl | acetyl | acetyl | H | acetyl | H | acetyl | acetyl | acetyl |
| 3490 | H | H | acetyl | acetyl | acetyl | H | acetyl | acetyl | H | H | H |
| 3491 | H | H | acetyl | acetyl | acetyl | H | acetyl | acetyl | H | H | acetyl |
| 3492 | H | H | acetyl | acetyl | acetyl | H | acetyl | acetyl | H | acetyl | H |
| 3493 | H | H | acetyl | acetyl | acetyl | H | acetyl | acetyl | H | acetyl | acetyl |
| 3494 | H | H | acetyl | acetyl | acetyl | H | acetyl | acetyl | acetyl | H | H |
| 3495 | H | H | acetyl | acetyl | acetyl | H | acetyl | acetyl | acetyl | H | acetyl |
| 3496 | H | H | acetyl | acetyl | acetyl | H | acetyl | acetyl | acetyl | acetyl | H |
| 3497 | H | H | acetyl | acetyl | acetyl | H | acetyl | acetyl | acetyl | acetyl | acetyl |
| 3498 | H | H | acetyl | acetyl | acetyl | acetyl | H | H | H | H | H |
| 3499 | H | H | acetyl | acetyl | acetyl | acetyl | H | H | H | H | acetyl |
| 3500 | H | H | acetyl | acetyl | acetyl | acetyl | H | H | H | acetyl | H |
| 3501 | H | H | acetyl | acetyl | acetyl | acetyl | H | H | H | acetyl | acetyl |
| 3502 | H | H | acetyl | acetyl | acetyl | acetyl | H | H | acetyl | H | H |
| 3503 | H | H | acetyl | acetyl | acetyl | acetyl | H | H | acetyl | H | acetyl |
| 3504 | H | H | acetyl | acetyl | acetyl | acetyl | H | H | acetyl | acetyl | H |
| 3505 | H | H | acetyl | acetyl | acetyl | acetyl | H | H | acetyl | acetyl | acetyl |
| 3506 | H | H | acetyl | acetyl | acetyl | acetyl | H | acetyl | H | H | H |
| 3507 | H | H | acetyl | acetyl | acetyl | acetyl | H | acetyl | H | H | acetyl |
| 3508 | H | H | acetyl | acetyl | acetyl | acetyl | H | acetyl | H | acetyl | H |
| 3509 | H | H | acetyl | acetyl | acetyl | acetyl | H | acetyl | H | acetyl | acetyl |
| 3510 | H | H | acetyl | acetyl | acetyl | acetyl | H | acetyl | acetyl | H | H |
| 3511 | H | H | acetyl | acetyl | acetyl | acetyl | H | acetyl | acetyl | H | acetyl |
| 3512 | H | H | acetyl | acetyl | acetyl | acetyl | H | acetyl | acetyl | acetyl | H |
| 3513 | H | H | acetyl | acetyl | acetyl | acetyl | H | acetyl | acetyl | acetyl | acetyl |
| 3514 | H | H | acetyl | acetyl | acetyl | acetyl | acetyl | H | H | H | H |
| 3515 | H | H | acetyl | acetyl | acetyl | acetyl | acetyl | H | H | H | acetyl |
| 3516 | H | H | acetyl | acetyl | acetyl | acetyl | acetyl | H | H | acetyl | H |
| 3517 | H | H | acetyl | acetyl | acetyl | acetyl | acetyl | H | H | acetyl | acetyl |
| 3518 | H | H | acetyl | acetyl | acetyl | acetyl | acetyl | H | acetyl | H | H |
| 3519 | H | H | acetyl | acetyl | acetyl | acetyl | acetyl | H | acetyl | H | acetyl |
| 3520 | H | H | acetyl | acetyl | acetyl | acetyl | acetyl | H | acetyl | acetyl | H |
| 3521 | H | H | acetyl | acetyl | acetyl | acetyl | acetyl | H | acetyl | acetyl | acetyl |
| 3522 | H | H | acetyl | acetyl | acetyl | acetyl | acetyl | acetyl | H | H | H |
| 3523 | H | H | acetyl | acetyl | acetyl | acetyl | acetyl | acetyl | H | H | acetyl |
| 3524 | H | H | acetyl | acetyl | acetyl | acetyl | acetyl | acetyl | H | acetyl | H |
| 3525 | H | H | acetyl | acetyl | acetyl | acetyl | acetyl | acetyl | H | acetyl | acetyl |
| 3526 | H | H | acetyl | acetyl | acetyl | acetyl | acetyl | acetyl | acetyl | H | H |
| 3527 | H | H | acetyl | acetyl | acetyl | acetyl | acetyl | acetyl | acetyl | H | acetyl |
| 3528 | H | H | acetyl | acetyl | acetyl | acetyl | acetyl | acetyl | acetyl | acetyl | H |
| 3529 | H | H | acetyl | acetyl | acetyl | acetyl | acetyl | acetyl | acetyl | acetyl | acetyl |
| 3530 | H | acetyl | H | H | H | H | H | H | H | H | H |
| 3531 | H | acetyl | H | H | H | H | H | H | H | H | acetyl |
| 3532 | H | acetyl | H | H | H | H | H | H | H | acetyl | H |
| 3533 | H | acetyl | H | H | H | H | H | H | H | acetyl | acetyl |
| 3534 | H | acetyl | H | H | H | H | H | H | acetyl | H | H |
| 3535 | H | acetyl | H | H | H | H | H | H | acetyl | H | acetyl |
| 3536 | H | acetyl | H | H | H | H | H | H | acetyl | acetyl | H |
| 3537 | H | acetyl | H | H | H | H | H | H | acetyl | acetyl | acetyl |
| 3538 | H | acetyl | H | H | H | H | H | acetyl | H | H | H |
| 3539 | H | acetyl | H | H | H | H | H | acetyl | H | H | acetyl |
| 3540 | H | acetyl | H | H | H | H | H | acetyl | H | acetyl | H |
| 3541 | H | acetyl | H | H | H | H | H | acetyl | H | acetyl | acetyl |
| 3542 | H | acetyl | H | H | H | H | H | acetyl | acetyl | H | H |
| 3543 | H | acetyl | H | H | H | H | H | acetyl | acetyl | H | acetyl |
| 3544 | H | acetyl | H | H | H | H | H | acetyl | acetyl | acetyl | H |
| 3545 | H | acetyl | H | H | H | H | H | acetyl | acetyl | acetyl | acetyl |
| 3546 | H | acetyl | H | H | H | H | acetyl | H | H | H | H |
| 3547 | H | acetyl | H | H | H | H | acetyl | H | H | H | acetyl |
| 3548 | H | acetyl | H | H | H | H | acetyl | H | H | acetyl | H |
| 3549 | H | acetyl | H | H | H | H | acetyl | H | H | acetyl | acetyl |
| 3550 | H | acetyl | H | H | H | H | acetyl | H | acetyl | H | H |
| 3551 | H | acetyl | H | H | H | H | acetyl | H | acetyl | H | acetyl |
| 3552 | H | acetyl | H | H | H | H | acetyl | H | acetyl | acetyl | H |
| 3553 | H | acetyl | H | H | H | H | acetyl | H | acetyl | acetyl | acetyl |
| 3554 | H | acetyl | H | H | H | H | acetyl | acetyl | H | H | H |
| 3555 | H | acetyl | H | H | H | H | acetyl | acetyl | H | H | acetyl |
| 3556 | H | acetyl | H | H | H | H | acetyl | acetyl | H | acetyl | H |
| 3557 | H | acetyl | H | H | H | H | acetyl | acetyl | H | acetyl | acetyl |
| 3558 | H | acetyl | H | H | H | H | acetyl | acetyl | acetyl | H | H |
| 3559 | H | acetyl | H | H | H | H | acetyl | acetyl | acetyl | H | acetyl |
| 3560 | H | acetyl | H | H | H | H | acetyl | acetyl | acetyl | acetyl | H |
| 3561 | H | acetyl | H | H | H | H | acetyl | acetyl | acetyl | acetyl | acetyl |
| 3562 | H | acetyl | H | H | H | acetyl | H | H | H | H | H |
| 3563 | H | acetyl | H | H | H | acetyl | H | H | H | H | acetyl |
| 3564 | H | acetyl | H | H | H | acetyl | H | H | H | acetyl | H |
| 3565 | H | acetyl | H | H | H | acetyl | H | H | H | acetyl | acetyl |
| 3566 | H | acetyl | H | H | H | acetyl | H | H | acetyl | H | H |
| 3567 | H | acetyl | H | H | H | acetyl | H | H | acetyl | H | acetyl |
| 3568 | H | acetyl | H | H | H | acetyl | H | H | acetyl | acetyl | H |
| 3569 | H | acetyl | H | H | H | acetyl | H | H | acetyl | acetyl | acetyl |
| 3570 | H | acetyl | H | H | H | acetyl | H | acetyl | H | H | H |
| 3571 | H | acetyl | H | H | H | acetyl | H | acetyl | H | H | acetyl |
| 3572 | H | acetyl | H | H | H | acetyl | H | acetyl | H | acetyl | H |
| 3573 | H | acetyl | H | H | H | acetyl | H | acetyl | H | acetyl | acetyl |
| 3574 | H | acetyl | H | H | H | acetyl | H | acetyl | acetyl | H | H |
| 3575 | H | acetyl | H | H | H | acetyl | H | acetyl | acetyl | H | acetyl |
| 3576 | H | acetyl | H | H | H | acetyl | H | acetyl | acetyl | acetyl | H |
| 3577 | H | acetyl | H | H | H | acetyl | H | acetyl | acetyl | acetyl | acetyl |
| 3578 | H | acetyl | H | H | H | acetyl | acetyl | H | H | H | H |
| 3579 | H | acetyl | H | H | H | acetyl | acetyl | H | H | H | acetyl |
| 3580 | H | acetyl | H | H | H | acetyl | acetyl | H | H | acetyl | H |
| 3581 | H | acetyl | H | H | H | acetyl | acetyl | H | H | acetyl | acetyl |
| 3582 | H | acetyl | H | H | H | acetyl | acetyl | H | acetyl | H | H |
| 3583 | H | acetyl | H | H | H | acetyl | acetyl | H | acetyl | H | acetyl |
| 3584 | H | acetyl | H | H | H | acetyl | acetyl | H | acetyl | acetyl | H |
| 3585 | H | acetyl | H | H | H | acetyl | acetyl | H | acetyl | acetyl | acetyl |
| 3586 | H | acetyl | H | H | H | acetyl | acetyl | acetyl | H | H | H |
| 3587 | H | acetyl | H | H | H | acetyl | acetyl | acetyl | H | H | acetyl |
| 3588 | H | acetyl | H | H | H | acetyl | acetyl | acetyl | H | acetyl | H |
| 3589 | H | acetyl | H | H | H | acetyl | acetyl | acetyl | H | acetyl | acetyl |
| 3590 | H | acetyl | H | H | H | acetyl | acetyl | acetyl | acetyl | H | H |
| 3591 | H | acetyl | H | H | H | acetyl | acetyl | acetyl | acetyl | H | acetyl |
| 3592 | H | acetyl | H | H | H | acetyl | acetyl | acetyl | acetyl | acetyl | H |
| 3593 | H | acetyl | H | H | H | acetyl | acetyl | acetyl | acetyl | acetyl | acetyl |
| 3594 | H | acetyl | H | H | acetyl | H | H | H | H | H | H |
| 3595 | H | acetyl | H | H | acetyl | H | H | H | H | H | acetyl |
| 3596 | H | acetyl | H | H | acetyl | H | H | H | H | acetyl | H |
| 3597 | H | acetyl | H | H | acetyl | H | H | H | H | acetyl | acetyl |
| 3598 | H | acetyl | H | H | acetyl | H | H | H | acetyl | H | H |
| 3599 | H | acetyl | H | H | acetyl | H | H | H | acetyl | H | acetyl |
| 3600 | H | acetyl | H | H | acetyl | H | H | H | acetyl | acetyl | H |
| 3601 | H | acetyl | H | H | acetyl | H | H | H | acetyl | acetyl | acetyl |
| 3602 | H | acetyl | H | H | acetyl | H | H | acetyl | H | H | H |
| 3603 | H | acetyl | H | H | acetyl | H | H | acetyl | H | H | acetyl |
| 3604 | H | acetyl | H | H | acetyl | H | H | acetyl | H | acetyl | H |
| 3605 | H | acetyl | H | H | acetyl | H | H | acetyl | H | acetyl | acetyl |
| 3606 | H | acetyl | H | H | acetyl | H | H | acetyl | acetyl | H | H |
| 3607 | H | acetyl | H | H | acetyl | H | H | acetyl | acetyl | H | acetyl |
| 3608 | H | acetyl | H | H | acetyl | H | H | acetyl | acetyl | acetyl | H |
| 3609 | H | acetyl | H | H | acetyl | H | H | acetyl | acetyl | acetyl | acetyl |
| 3610 | H | acetyl | H | H | acetyl | H | acetyl | H | H | H | H |
| 3611 | H | acetyl | H | H | acetyl | H | acetyl | H | H | H | acetyl |
| 3612 | H | acetyl | H | H | acetyl | H | acetyl | H | H | acetyl | H |
| 3613 | H | acetyl | H | H | acetyl | H | acetyl | H | H | acetyl | acetyl |
| 3614 | H | acetyl | H | H | acetyl | H | acetyl | H | acetyl | H | H |
| 3615 | H | acetyl | H | H | acetyl | H | acetyl | H | acetyl | H | acetyl |
| 3616 | H | acetyl | H | H | acetyl | H | acetyl | H | acetyl | acetyl | H |
| 3617 | H | acetyl | H | H | acetyl | H | acetyl | H | acetyl | acetyl | acetyl |
| 3618 | H | acetyl | H | H | acetyl | H | acetyl | acetyl | H | H | H |
| 3619 | H | acetyl | H | H | acetyl | H | acetyl | acetyl | H | H | acetyl |
| 3620 | H | acetyl | H | H | acetyl | H | acetyl | acetyl | H | acetyl | H |
| 3621 | H | acetyl | H | H | acetyl | H | acetyl | acetyl | H | acetyl | acetyl |
| 3622 | H | acetyl | H | H | acetyl | H | acetyl | acetyl | acetyl | H | H |
| 3623 | H | acetyl | H | H | acetyl | H | acetyl | acetyl | acetyl | H | acetyl |
| 3624 | H | acetyl | H | H | acetyl | H | acetyl | acetyl | acetyl | acetyl | H |
| 3625 | H | acetyl | H | H | acetyl | H | acetyl | acetyl | acetyl | acetyl | acetyl |
| 3626 | H | acetyl | H | H | acetyl | acetyl | H | H | H | H | H |
| 3627 | H | acetyl | H | H | acetyl | acetyl | H | H | H | H | acetyl |
| 3628 | H | acetyl | H | H | acetyl | acetyl | H | H | H | acetyl | H |
| 3629 | H | acetyl | H | H | acetyl | acetyl | H | H | H | acetyl | acetyl |
| 3630 | H | acetyl | H | H | acetyl | acetyl | H | H | acetyl | H | H |
| 3631 | H | acetyl | H | H | acetyl | acetyl | H | H | acetyl | H | acetyl |
| 3632 | H | acetyl | H | H | acetyl | acetyl | H | H | acetyl | acetyl | H |
| 3633 | H | acetyl | H | H | acetyl | acetyl | H | H | acetyl | acetyl | acetyl |
| 3634 | H | acetyl | H | H | acetyl | acetyl | H | acetyl | H | H | H |
| 3635 | H | acetyl | H | H | acetyl | acetyl | H | acetyl | H | H | acetyl |
| 3636 | H | acetyl | H | H | acetyl | acetyl | H | acetyl | H | acetyl | H |
| 3637 | H | acetyl | H | H | acetyl | acetyl | H | acetyl | H | acetyl | acetyl |
| 3638 | H | acetyl | H | H | acetyl | acetyl | H | acetyl | acetyl | H | H |
| 3639 | H | acetyl | H | H | acetyl | acetyl | H | acetyl | acetyl | H | acetyl |
| 3640 | H | acetyl | H | H | acetyl | acetyl | H | acetyl | acetyl | acetyl | H |
| 3641 | H | acetyl | H | H | acetyl | acetyl | H | acetyl | acetyl | acetyl | acetyl |
| 3642 | H | acetyl | H | H | acetyl | acetyl | acetyl | H | H | H | H |
| 3643 | H | acetyl | H | H | acetyl | acetyl | acetyl | H | H | H | acetyl |
| 3644 | H | acetyl | H | H | acetyl | acetyl | acetyl | H | H | acetyl | H |
| 3645 | H | acetyl | H | H | acetyl | acetyl | acetyl | H | H | acetyl | acetyl |
| 3646 | H | acetyl | H | H | acetyl | acetyl | acetyl | H | acetyl | H | H |
| 3647 | H | acetyl | H | H | acetyl | acetyl | acetyl | H | acetyl | H | acetyl |
| 3648 | H | acetyl | H | H | acetyl | acetyl | acetyl | H | acetyl | acetyl | H |
| 3649 | H | acetyl | H | H | acetyl | acetyl | acetyl | H | acetyl | acetyl | acetyl |
| 3650 | H | acetyl | H | H | acetyl | acetyl | acetyl | acetyl | H | H | H |
| 3651 | H | acetyl | H | H | acetyl | acetyl | acetyl | acetyl | H | H | acetyl |
| 3652 | H | acetyl | H | H | acetyl | acetyl | acetyl | acetyl | H | acetyl | H |
| 3653 | H | acetyl | H | H | acetyl | acetyl | acetyl | acetyl | H | acetyl | acetyl |
| 3654 | H | acetyl | H | H | acetyl | acetyl | acetyl | acetyl | acetyl | H | H |
| 3655 | H | acetyl | H | H | acetyl | acetyl | acetyl | acetyl | acetyl | H | acetyl |
| 3656 | H | acetyl | H | H | acetyl | acetyl | acetyl | acetyl | acetyl | acetyl | H |
| 3657 | H | acetyl | H | H | acetyl | acetyl | acetyl | acetyl | acetyl | acetyl | acetyl |
| 3658 | H | acetyl | H | acetyl | H | H | H | H | H | H | H |
| 3659 | H | acetyl | H | acetyl | H | H | H | H | H | H | acetyl |
| 3660 | H | acetyl | H | acetyl | H | H | H | H | H | acetyl | H |
| 3661 | H | acetyl | H | acetyl | H | H | H | H | H | acetyl | acetyl |
| 3662 | H | acetyl | H | acetyl | H | H | H | H | acetyl | H | H |
| 3663 | H | acetyl | H | acetyl | H | H | H | H | acetyl | H | acetyl |
| 3664 | H | acetyl | H | acetyl | H | H | H | H | acetyl | acetyl | H |
| 3665 | H | acetyl | H | acetyl | H | H | H | H | acetyl | acetyl | acetyl |
| 3666 | H | acetyl | H | acetyl | H | H | H | acetyl | H | H | H |
| 3667 | H | acetyl | H | acetyl | H | H | H | acetyl | H | H | acetyl |
| 3668 | H | acetyl | H | acetyl | H | H | H | acetyl | H | acetyl | H |
| 3669 | H | acetyl | H | acetyl | H | H | H | acetyl | H | acetyl | acetyl |
| 3670 | H | acetyl | H | acetyl | H | H | H | acetyl | acetyl | H | H |
| 3671 | H | acetyl | H | acetyl | H | H | H | acetyl | acetyl | H | acetyl |
| 3672 | H | acetyl | H | acetyl | H | H | H | acetyl | acetyl | acetyl | H |
| 3673 | H | acetyl | H | acetyl | H | H | H | acetyl | acetyl | acetyl | acetyl |
| 3674 | H | acetyl | H | acetyl | H | H | acetyl | H | H | H | H |
| 3675 | H | acetyl | H | acetyl | H | H | acetyl | H | H | H | acetyl |
| 3676 | H | acetyl | H | acetyl | H | H | acetyl | H | H | acetyl | H |
| 3677 | H | acetyl | H | acetyl | H | H | acetyl | H | H | acetyl | acetyl |
| 3678 | H | acetyl | H | acetyl | H | H | acetyl | H | acetyl | H | H |
| 3679 | H | acetyl | H | acetyl | H | H | acetyl | H | acetyl | H | acetyl |
| 3680 | H | acetyl | H | acetyl | H | H | acetyl | H | acetyl | acetyl | H |
| 3681 | H | acetyl | H | acetyl | H | H | acetyl | H | acetyl | acetyl | acetyl |
| 3682 | H | acetyl | H | acetyl | H | H | acetyl | acetyl | H | H | H |
| 3683 | H | acetyl | H | acetyl | H | H | acetyl | acetyl | H | H | acetyl |
| 3684 | H | acetyl | H | acetyl | H | H | acetyl | acetyl | H | acetyl | H |
| 3685 | H | acetyl | H | acetyl | H | H | acetyl | acetyl | H | acetyl | acetyl |
| 3686 | H | acetyl | H | acetyl | H | H | acetyl | acetyl | acetyl | H | H |
| 3687 | H | acetyl | H | acetyl | H | H | acetyl | acetyl | acetyl | H | acetyl |
| 3688 | H | acetyl | H | acetyl | H | H | acetyl | acetyl | acetyl | acetyl | H |
| 3689 | H | acetyl | H | acetyl | H | H | acetyl | acetyl | acetyl | acetyl | acetyl |
| 3690 | H | acetyl | H | acetyl | H | acetyl | H | H | H | H | H |
| 3691 | H | acetyl | H | acetyl | H | acetyl | H | H | H | H | acetyl |
| 3692 | H | acetyl | H | acetyl | H | acetyl | H | H | H | acetyl | H |
| 3693 | H | acetyl | H | acetyl | H | acetyl | H | H | H | acetyl | acetyl |
| 3694 | H | acetyl | H | acetyl | H | acetyl | H | H | acetyl | H | H |
| 3695 | H | acetyl | H | acetyl | H | acetyl | H | H | acetyl | H | acetyl |
| 3696 | H | acetyl | H | acetyl | H | acetyl | H | H | acetyl | acetyl | H |
| 3697 | H | acetyl | H | acetyl | H | acetyl | H | H | acetyl | acetyl | acetyl |
| 3698 | H | acetyl | H | acetyl | H | acetyl | H | acetyl | H | H | H |
| 3699 | H | acetyl | H | acetyl | H | acetyl | H | acetyl | H | H | acetyl |
| 3700 | H | acetyl | H | acetyl | H | acetyl | H | acetyl | H | acetyl | H |
| 3701 | H | acetyl | H | acetyl | H | acetyl | H | acetyl | H | acetyl | acetyl |
| 3702 | H | acetyl | H | acetyl | H | acetyl | H | acetyl | acetyl | H | H |
| 3703 | H | acetyl | H | acetyl | H | acetyl | H | acetyl | acetyl | H | acetyl |
| 3704 | H | acetyl | H | acetyl | H | acetyl | H | acetyl | acetyl | acetyl | H |
| 3705 | H | acetyl | H | acetyl | H | acetyl | H | acetyl | acetyl | acetyl | acetyl |
| 3706 | H | acetyl | H | acetyl | H | acetyl | acetyl | H | H | H | H |
| 3707 | H | acetyl | H | acetyl | H | acetyl | acetyl | H | H | H | acetyl |
| 3708 | H | acetyl | H | acetyl | H | acetyl | acetyl | H | H | acetyl | H |
| 3709 | H | acetyl | H | acetyl | H | acetyl | acetyl | H | H | acetyl | acetyl |
| 3710 | H | acetyl | H | acetyl | H | acetyl | acetyl | H | acetyl | H | H |
| 3711 | H | acetyl | H | acetyl | H | acetyl | acetyl | H | acetyl | H | acetyl |
| 3712 | H | acetyl | H | acetyl | H | acetyl | acetyl | H | acetyl | acetyl | H |
| 3713 | H | acetyl | H | acetyl | H | acetyl | acetyl | H | acetyl | acetyl | acetyl |
| 3714 | H | acetyl | H | acetyl | H | acetyl | acetyl | acetyl | H | H | H |
| 3715 | H | acetyl | H | acetyl | H | acetyl | acetyl | acetyl | H | H | acetyl |
| 3716 | H | acetyl | H | acetyl | H | acetyl | acetyl | acetyl | H | acetyl | H |
| 3717 | H | acetyl | H | acetyl | H | acetyl | acetyl | acetyl | H | acetyl | acetyl |
| 3718 | H | acetyl | H | acetyl | H | acetyl | acetyl | acetyl | acetyl | H | H |
| 3719 | H | acetyl | H | acetyl | H | acetyl | acetyl | acetyl | acetyl | H | acetyl |
| 3720 | H | acetyl | H | acetyl | H | acetyl | acetyl | acetyl | acetyl | acetyl | H |
| 3721 | H | acetyl | H | acetyl | H | acetyl | acetyl | acetyl | acetyl | acetyl | acetyl |
| 3722 | H | acetyl | H | acetyl | acetyl | H | H | H | H | H | H |
| 3723 | H | acetyl | H | acetyl | acetyl | H | H | H | H | H | acetyl |
| 3724 | H | acetyl | H | acetyl | acetyl | H | H | H | H | acetyl | H |
| 3725 | H | acetyl | H | acetyl | acetyl | H | H | H | H | acetyl | acetyl |
| 3726 | H | acetyl | H | acetyl | acetyl | H | H | H | acetyl | H | H |
| 3727 | H | acetyl | H | acetyl | acetyl | H | H | H | acetyl | H | acetyl |
| 3728 | H | acetyl | H | acetyl | acetyl | H | H | H | acetyl | acetyl | H |
| 3729 | H | acetyl | H | acetyl | acetyl | H | H | H | acetyl | acetyl | acetyl |
| 3730 | H | acetyl | H | acetyl | acetyl | H | H | acetyl | H | H | H |
| 3731 | H | acetyl | H | acetyl | acetyl | H | H | acetyl | H | H | acetyl |
| 3732 | H | acetyl | H | acetyl | acetyl | H | H | acetyl | H | acetyl | H |
| 3733 | H | acetyl | H | acetyl | acetyl | H | H | acetyl | H | acetyl | acetyl |
| 3734 | H | acetyl | H | acetyl | acetyl | H | H | acetyl | acetyl | H | H |
| 3735 | H | acetyl | H | acetyl | acetyl | H | H | acetyl | acetyl | H | acetyl |
| 3736 | H | acetyl | H | acetyl | acetyl | H | H | acetyl | acetyl | acetyl | H |
| 3737 | H | acetyl | H | acetyl | acetyl | H | H | acetyl | acetyl | acetyl | acetyl |
| 3738 | H | acetyl | H | acetyl | acetyl | H | acetyl | H | H | H | H |
| 3739 | H | acetyl | H | acetyl | acetyl | H | acetyl | H | H | H | acetyl |
| 3740 | H | acetyl | H | acetyl | acetyl | H | acetyl | H | H | acetyl | H |
| 3741 | H | acetyl | H | acetyl | acetyl | H | acetyl | H | H | acetyl | acetyl |
| 3742 | H | acetyl | H | acetyl | acetyl | H | acetyl | H | acetyl | H | H |
| 3743 | H | acetyl | H | acetyl | acetyl | H | acetyl | H | acetyl | H | acetyl |
| 3744 | H | acetyl | H | acetyl | acetyl | H | acetyl | H | acetyl | acetyl | H |
| 3745 | H | acetyl | H | acetyl | acetyl | H | acetyl | H | acetyl | acetyl | acetyl |
| 3746 | H | acetyl | H | acetyl | acetyl | H | acetyl | acetyl | H | H | H |
| 3747 | H | acetyl | H | acetyl | acetyl | H | acetyl | acetyl | H | H | acetyl |
| 3748 | H | acetyl | H | acetyl | acetyl | H | acetyl | acetyl | H | acetyl | H |
| 3749 | H | acetyl | H | acetyl | acetyl | H | acetyl | acetyl | H | acetyl | acetyl |
| 3750 | H | acetyl | H | acetyl | acetyl | H | acetyl | acetyl | acetyl | H | H |
| 3751 | H | acetyl | H | acetyl | acetyl | H | acetyl | acetyl | acetyl | H | acetyl |
| 3752 | H | acetyl | H | acetyl | acetyl | H | acetyl | acetyl | acetyl | acetyl | H |
| 3753 | H | acetyl | H | acetyl | acetyl | H | acetyl | acetyl | acetyl | acetyl | acetyl |
| 3754 | H | acetyl | H | acetyl | acetyl | acetyl | H | H | H | H | H |
| 3755 | H | acetyl | H | acetyl | acetyl | acetyl | H | H | H | H | acetyl |
| 3756 | H | acetyl | H | acetyl | acetyl | acetyl | H | H | H | acetyl | H |
| 3757 | H | acetyl | H | acetyl | acetyl | acetyl | H | H | H | acetyl | acetyl |
| 3758 | H | acetyl | H | acetyl | acetyl | acetyl | H | H | acetyl | H | H |
| 3759 | H | acetyl | H | acetyl | acetyl | acetyl | H | H | acetyl | H | acetyl |
| 3760 | H | acetyl | H | acetyl | acetyl | acetyl | H | H | acetyl | acetyl | H |
| 3761 | H | acetyl | H | acetyl | acetyl | acetyl | H | H | acetyl | acetyl | acetyl |
| 3762 | H | acetyl | H | acetyl | acetyl | acetyl | H | acetyl | H | H | H |
| 3763 | H | acetyl | H | acetyl | acetyl | acetyl | H | acetyl | H | H | acetyl |
| 3764 | H | acetyl | H | acetyl | acetyl | acetyl | H | acetyl | H | acetyl | H |
| 3765 | H | acetyl | H | acetyl | acetyl | acetyl | H | acetyl | H | acetyl | acetyl |
| 3766 | H | acetyl | H | acetyl | acetyl | acetyl | H | acetyl | acetyl | H | H |
| 3767 | H | acetyl | H | acetyl | acetyl | acetyl | H | acetyl | acetyl | H | acetyl |
| 3768 | H | acetyl | H | acetyl | acetyl | acetyl | H | acetyl | acetyl | acetyl | H |
| 3769 | H | acetyl | H | acetyl | acetyl | acetyl | H | acetyl | acetyl | acetyl | acetyl |
| 3770 | H | acetyl | H | acetyl | acetyl | acetyl | acetyl | H | H | H | H |
| 3771 | H | acetyl | H | acetyl | acetyl | acetyl | acetyl | H | H | H | acetyl |
| 3772 | H | acetyl | H | acetyl | acetyl | acetyl | acetyl | H | H | acetyl | H |
| 3773 | H | acetyl | H | acetyl | acetyl | acetyl | acetyl | H | H | acetyl | acetyl |
| 3774 | H | acetyl | H | acetyl | acetyl | acetyl | acetyl | H | acetyl | H | H |
| 3775 | H | acetyl | H | acetyl | acetyl | acetyl | acetyl | H | acetyl | H | acetyl |
| 3776 | H | acetyl | H | acetyl | acetyl | acetyl | acetyl | H | acetyl | acetyl | H |
| 3777 | H | acetyl | H | acetyl | acetyl | acetyl | acetyl | H | acetyl | acetyl | acetyl |
| 3778 | H | acetyl | H | acetyl | acetyl | acetyl | acetyl | acetyl | H | H | H |
| 3779 | H | acetyl | H | acetyl | acetyl | acetyl | acetyl | acetyl | H | H | acetyl |
| 3780 | H | acetyl | H | acetyl | acetyl | acetyl | acetyl | acetyl | H | acetyl | H |
| 3781 | H | acetyl | H | acetyl | acetyl | acetyl | acetyl | acetyl | H | acetyl | acetyl |
| 3782 | H | acetyl | H | acetyl | acetyl | acetyl | acetyl | acetyl | acetyl | H | H |
| 3783 | H | acetyl | H | acetyl | acetyl | acetyl | acetyl | acetyl | acetyl | H | acetyl |
| 3784 | H | acetyl | H | acetyl | acetyl | acetyl | acetyl | acetyl | acetyl | acetyl | H |
| 3785 | H | acetyl | H | acetyl | acetyl | acetyl | acetyl | acetyl | acetyl | acetyl | acetyl |
| 3786 | H | acetyl | acetyl | H | H | H | H | H | H | H | H |
| 3787 | H | acetyl | acetyl | H | H | H | H | H | H | H | acetyl |
| 3788 | H | acetyl | acetyl | H | H | H | H | H | H | acetyl | H |
| 3789 | H | acetyl | acetyl | H | H | H | H | H | H | acetyl | acetyl |
| 3790 | H | acetyl | acetyl | H | H | H | H | H | acetyl | H | H |
| 3791 | H | acetyl | acetyl | H | H | H | H | H | acetyl | H | acetyl |
| 3792 | H | acetyl | acetyl | H | H | H | H | H | acetyl | acetyl | H |
| 3793 | H | acetyl | acetyl | H | H | H | H | H | acetyl | acetyl | acetyl |
| 3794 | H | acetyl | acetyl | H | H | H | H | acetyl | H | H | H |
| 3795 | H | acetyl | acetyl | H | H | H | H | acetyl | H | H | acetyl |
| 3796 | H | acetyl | acetyl | H | H | H | H | acetyl | H | acetyl | H |
| 3797 | H | acetyl | acetyl | H | H | H | H | acetyl | H | acetyl | acetyl |
| 3798 | H | acetyl | acetyl | H | H | H | H | acetyl | acetyl | H | H |
| 3799 | H | acetyl | acetyl | H | H | H | H | acetyl | acetyl | H | acetyl |
| 3800 | H | acetyl | acetyl | H | H | H | H | acetyl | acetyl | acetyl | H |
| 3801 | H | acetyl | acetyl | H | H | H | H | acetyl | acetyl | acetyl | acetyl |
| 3802 | H | acetyl | acetyl | H | H | H | acetyl | H | H | H | H |
| 3803 | H | acetyl | acetyl | H | H | H | acetyl | H | H | H | acetyl |
| 3804 | H | acetyl | acetyl | H | H | H | acetyl | H | H | acetyl | H |
| 3805 | H | acetyl | acetyl | H | H | H | acetyl | H | H | acetyl | acetyl |
| 3806 | H | acetyl | acetyl | H | H | H | acetyl | H | acetyl | H | H |
| 3807 | H | acetyl | acetyl | H | H | H | acetyl | H | acetyl | H | acetyl |
| 3808 | H | acetyl | acetyl | H | H | H | acetyl | H | acetyl | acetyl | H |
| 3809 | H | acetyl | acetyl | H | H | H | acetyl | H | acetyl | acetyl | acetyl |
| 3810 | H | acetyl | acetyl | H | H | H | acetyl | acetyl | H | H | H |
| 3811 | H | acetyl | acetyl | H | H | H | acetyl | acetyl | H | H | acetyl |
| 3812 | H | acetyl | acetyl | H | H | H | acetyl | acetyl | H | acetyl | H |
| 3813 | H | acetyl | acetyl | H | H | H | acetyl | acetyl | H | acetyl | acetyl |
| 3814 | H | acetyl | acetyl | H | H | H | acetyl | acetyl | acetyl | H | H |
| 3815 | H | acetyl | acetyl | H | H | H | acetyl | acetyl | acetyl | H | acetyl |
| 3816 | H | acetyl | acetyl | H | H | H | acetyl | acetyl | acetyl | acetyl | H |
| 3817 | H | acetyl | acetyl | H | H | H | acetyl | acetyl | acetyl | acetyl | acetyl |
| 3818 | H | acetyl | acetyl | H | H | acetyl | H | H | H | H | H |
| 3819 | H | acetyl | acetyl | H | H | acetyl | H | H | H | H | acetyl |
| 3820 | H | acetyl | acetyl | H | H | acetyl | H | H | H | acetyl | H |
| 3821 | H | acetyl | acetyl | H | H | acetyl | H | H | H | acetyl | acetyl |
| 3822 | H | acetyl | acetyl | H | H | acetyl | H | H | acetyl | H | H |
| 3823 | H | acetyl | acetyl | H | H | acetyl | H | H | acetyl | H | acetyl |
| 3824 | H | acetyl | acetyl | H | H | acetyl | H | H | acetyl | acetyl | H |
| 3825 | H | acetyl | acetyl | H | H | acetyl | H | H | acetyl | acetyl | acetyl |
| 3826 | H | acetyl | acetyl | H | H | acetyl | H | acetyl | H | H | H |
| 3827 | H | acetyl | acetyl | H | H | acetyl | H | acetyl | H | H | acetyl |
| 3828 | H | acetyl | acetyl | H | H | acetyl | H | acetyl | H | acetyl | H |
| 3829 | H | acetyl | acetyl | H | H | acetyl | H | acetyl | H | acetyl | acetyl |
| 3830 | H | acetyl | acetyl | H | H | acetyl | H | acetyl | acetyl | H | H |
| 3831 | H | acetyl | acetyl | H | H | acetyl | H | acetyl | acetyl | H | acetyl |
| 3832 | H | acetyl | acetyl | H | H | acetyl | H | acetyl | acetyl | acetyl | H |
| 3833 | H | acetyl | acetyl | H | H | acetyl | H | acetyl | acetyl | acetyl | acetyl |
| 3834 | H | acetyl | acetyl | H | H | acetyl | acetyl | H | H | H | H |
| 3835 | H | acetyl | acetyl | H | H | acetyl | acetyl | H | H | H | acetyl |
| 3836 | H | acetyl | acetyl | H | H | acetyl | acetyl | H | H | acetyl | H |
| 3837 | H | acetyl | acetyl | H | H | acetyl | acetyl | H | H | acetyl | acetyl |
| 3838 | H | acetyl | acetyl | H | H | acetyl | acetyl | H | acetyl | H | H |
| 3839 | H | acetyl | acetyl | H | H | acetyl | acetyl | H | acetyl | H | acetyl |
| 3840 | H | acetyl | acetyl | H | H | acetyl | acetyl | H | acetyl | acetyl | H |
| 3841 | H | acetyl | acetyl | H | H | acetyl | acetyl | H | acetyl | acetyl | acetyl |
| 3842 | H | acetyl | acetyl | H | H | acetyl | acetyl | acetyl | H | H | H |
| 3843 | H | acetyl | acetyl | H | H | acetyl | acetyl | acetyl | H | H | acetyl |
| 3844 | H | acetyl | acetyl | H | H | acetyl | acetyl | acetyl | H | acetyl | H |
| 3845 | H | acetyl | acetyl | H | H | acetyl | acetyl | acetyl | H | acetyl | acetyl |
| 3846 | H | acetyl | acetyl | H | H | acetyl | acetyl | acetyl | acetyl | H | H |
| 3847 | H | acetyl | acetyl | H | H | acetyl | acetyl | acetyl | acetyl | H | acetyl |
| 3848 | H | acetyl | acetyl | H | H | acetyl | acetyl | acetyl | acetyl | acetyl | H |
| 3849 | H | acetyl | acetyl | H | H | acetyl | acetyl | acetyl | acetyl | acetyl | acetyl |
| 3850 | H | acetyl | acetyl | H | acetyl | H | H | H | H | H | H |
| 3851 | H | acetyl | acetyl | H | acetyl | H | H | H | H | H | acetyl |
| 3852 | H | acetyl | acetyl | H | acetyl | H | H | H | H | acetyl | H |
| 3853 | H | acetyl | acetyl | H | acetyl | H | H | H | H | acetyl | acetyl |
| 3854 | H | acetyl | acetyl | H | acetyl | H | H | H | acetyl | H | H |
| 3855 | H | acetyl | acetyl | H | acetyl | H | H | H | acetyl | H | acetyl |
| 3856 | H | acetyl | acetyl | H | acetyl | H | H | H | acetyl | acetyl | H |
| 3857 | H | acetyl | acetyl | H | acetyl | H | H | H | acetyl | acetyl | acetyl |
| 3858 | H | acetyl | acetyl | H | acetyl | H | H | acetyl | H | H | H |
| 3859 | H | acetyl | acetyl | H | acetyl | H | H | acetyl | H | H | acetyl |
| 3860 | H | acetyl | acetyl | H | acetyl | H | H | acetyl | H | acetyl | H |
| 3861 | H | acetyl | acetyl | H | acetyl | H | H | acetyl | H | acetyl | acetyl |
| 3862 | H | acetyl | acetyl | H | acetyl | H | H | acetyl | acetyl | H | H |
| 3863 | H | acetyl | acetyl | H | acetyl | H | H | acetyl | acetyl | H | acetyl |
| 3864 | H | acetyl | acetyl | H | acetyl | H | H | acetyl | acetyl | acetyl | H |
| 3865 | H | acetyl | acetyl | H | acetyl | H | H | acetyl | acetyl | acetyl | acetyl |
| 3866 | H | acetyl | acetyl | H | acetyl | H | acetyl | H | H | H | H |
| 3867 | H | acetyl | acetyl | H | acetyl | H | acetyl | H | H | H | acetyl |
| 3868 | H | acetyl | acetyl | H | acetyl | H | acetyl | H | H | acetyl | H |
| 3869 | H | acetyl | acetyl | H | acetyl | H | acetyl | H | H | acetyl | acetyl |
| 3870 | H | acetyl | acetyl | H | acetyl | H | acetyl | H | acetyl | H | H |
| 3871 | H | acetyl | acetyl | H | acetyl | H | acetyl | H | acetyl | H | acetyl |
| 3872 | H | acetyl | acetyl | H | acetyl | H | acetyl | H | acetyl | acetyl | H |
| 3873 | H | acetyl | acetyl | H | acetyl | H | acetyl | H | acetyl | acetyl | acetyl |
| 3874 | H | acetyl | acetyl | H | acetyl | H | acetyl | acetyl | H | H | H |
| 3875 | H | acetyl | acetyl | H | acetyl | H | acetyl | acetyl | H | H | acetyl |
| 3876 | H | acetyl | acetyl | H | acetyl | H | acetyl | acetyl | H | acetyl | H |
| 3877 | H | acetyl | acetyl | H | acetyl | H | acetyl | acetyl | H | acetyl | acetyl |
| 3878 | H | acetyl | acetyl | H | acetyl | H | acetyl | acetyl | acetyl | H | H |
| 3879 | H | acetyl | acetyl | H | acetyl | H | acetyl | acetyl | acetyl | H | acetyl |
| 3880 | H | acetyl | acetyl | H | acetyl | H | acetyl | acetyl | acetyl | acetyl | H |
| 3881 | H | acetyl | acetyl | H | acetyl | H | acetyl | acetyl | acetyl | acetyl | acetyl |
| 3882 | H | acetyl | acetyl | H | acetyl | acetyl | H | H | H | H | H |
| 3883 | H | acetyl | acetyl | H | acetyl | acetyl | H | H | H | H | acetyl |
| 3884 | H | acetyl | acetyl | H | acetyl | acetyl | H | H | H | acetyl | H |
| 3885 | H | acetyl | acetyl | H | acetyl | acetyl | H | H | H | acetyl | acetyl |
| 3886 | H | acetyl | acetyl | H | acetyl | acetyl | H | H | acetyl | H | H |
| 3887 | H | acetyl | acetyl | H | acetyl | acetyl | H | H | acetyl | H | acetyl |
| 3888 | H | acetyl | acetyl | H | acetyl | acetyl | H | H | acetyl | acetyl | H |
| 3889 | H | acetyl | acetyl | H | acetyl | acetyl | H | H | acetyl | acetyl | acetyl |
| 3890 | H | acetyl | acetyl | H | acetyl | acetyl | H | acetyl | H | H | H |
| 3891 | H | acetyl | acetyl | H | acetyl | acetyl | H | acetyl | H | H | acetyl |
| 3892 | H | acetyl | acetyl | H | acetyl | acetyl | H | acetyl | H | acetyl | H |
| 3893 | H | acetyl | acetyl | H | acetyl | acetyl | H | acetyl | H | acetyl | acetyl |
| 3894 | H | acetyl | acetyl | H | acetyl | acetyl | H | acetyl | acetyl | H | H |
| 3895 | H | acetyl | acetyl | H | acetyl | acetyl | H | acetyl | acetyl | H | acetyl |
| 3896 | H | acetyl | acetyl | H | acetyl | acetyl | H | acetyl | acetyl | acetyl | H |
| 3897 | H | acetyl | acetyl | H | acetyl | acetyl | H | acetyl | acetyl | acetyl | acetyl |
| 3898 | H | acetyl | acetyl | H | acetyl | acetyl | acetyl | H | H | H | H |
| 3899 | H | acetyl | acetyl | H | acetyl | acetyl | acetyl | H | H | H | acetyl |
| 3900 | H | acetyl | acetyl | H | acetyl | acetyl | acetyl | H | H | acetyl | H |
| 3901 | H | acetyl | acetyl | H | acetyl | acetyl | acetyl | H | H | acetyl | acetyl |
| 3902 | H | acetyl | acetyl | H | acetyl | acetyl | acetyl | H | acetyl | H | H |
| 3903 | H | acetyl | acetyl | H | acetyl | acetyl | acetyl | H | acetyl | H | acetyl |
| 3904 | H | acetyl | acetyl | H | acetyl | acetyl | acetyl | H | acetyl | acetyl | H |
| 3905 | H | acetyl | acetyl | H | acetyl | acetyl | acetyl | H | acetyl | acetyl | acetyl |
| 3906 | H | acetyl | acetyl | H | acetyl | acetyl | acetyl | acetyl | H | H | H |
| 3907 | H | acetyl | acetyl | H | acetyl | acetyl | acetyl | acetyl | H | H | acetyl |
| 3908 | H | acetyl | acetyl | H | acetyl | acetyl | acetyl | acetyl | H | acetyl | H |
| 3909 | H | acetyl | acetyl | H | acetyl | acetyl | acetyl | acetyl | H | acetyl | acetyl |
| 3910 | H | acetyl | acetyl | H | acetyl | acetyl | acetyl | acetyl | acetyl | H | H |
| 3911 | H | acetyl | acetyl | H | acetyl | acetyl | acetyl | acetyl | acetyl | H | acetyl |
| 3912 | H | acetyl | acetyl | H | acetyl | acetyl | acetyl | acetyl | acetyl | acetyl | H |
| 3913 | H | acetyl | acetyl | H | acetyl | acetyl | acetyl | acetyl | acetyl | acetyl | acetyl |
| 3914 | H | acetyl | acetyl | acetyl | H | H | H | H | H | H | H |
| 3915 | H | acetyl | acetyl | acetyl | H | H | H | H | H | H | acetyl |
| 3916 | H | acetyl | acetyl | acetyl | H | H | H | H | H | acetyl | H |
| 3917 | H | acetyl | acetyl | acetyl | H | H | H | H | H | acetyl | acetyl |
| 3918 | H | acetyl | acetyl | acetyl | H | H | H | H | acetyl | H | H |
| 3919 | H | acetyl | acetyl | acetyl | H | H | H | H | acetyl | H | acetyl |
| 3920 | H | acetyl | acetyl | acetyl | H | H | H | H | acetyl | acetyl | H |
| 3921 | H | acetyl | acetyl | acetyl | H | H | H | H | acetyl | acetyl | acetyl |
| 3922 | H | acetyl | acetyl | acetyl | H | H | H | acetyl | H | H | H |
| 3923 | H | acetyl | acetyl | acetyl | H | H | H | acetyl | H | H | acetyl |
| 3924 | H | acetyl | acetyl | acetyl | H | H | H | acetyl | H | acetyl | H |
| 3925 | H | acetyl | acetyl | acetyl | H | H | H | acetyl | H | acetyl | acetyl |
| 3926 | H | acetyl | acetyl | acetyl | H | H | H | acetyl | acetyl | H | H |
| 3927 | H | acetyl | acetyl | acetyl | H | H | H | acetyl | acetyl | H | acetyl |
| 3928 | H | acetyl | acetyl | acetyl | H | H | H | acetyl | acetyl | acetyl | H |
| 3929 | H | acetyl | acetyl | acetyl | H | H | H | acetyl | acetyl | acetyl | acetyl |
| 3930 | H | acetyl | acetyl | acetyl | H | H | acetyl | H | H | H | H |
| 3931 | H | acetyl | acetyl | acetyl | H | H | acetyl | H | H | H | acetyl |
| 3932 | H | acetyl | acetyl | acetyl | H | H | acetyl | H | H | acetyl | H |
| 3933 | H | acetyl | acetyl | acetyl | H | H | acetyl | H | H | acetyl | acetyl |
| 3934 | H | acetyl | acetyl | acetyl | H | H | acetyl | H | acetyl | H | H |
| 3935 | H | acetyl | acetyl | acetyl | H | H | acetyl | H | acetyl | H | acetyl |
| 3936 | H | acetyl | acetyl | acetyl | H | H | acetyl | H | acetyl | acetyl | H |
| 3937 | H | acetyl | acetyl | acetyl | H | H | acetyl | H | acetyl | acetyl | acetyl |
| 3938 | H | acetyl | acetyl | acetyl | H | H | acetyl | acetyl | H | H | H |
| 3939 | H | acetyl | acetyl | acetyl | H | H | acetyl | acetyl | H | H | acetyl |
| 3940 | H | acetyl | acetyl | acetyl | H | H | acetyl | acetyl | H | acetyl | H |
| 3941 | H | acetyl | acetyl | acetyl | H | H | acetyl | acetyl | H | acetyl | acetyl |
| 3942 | H | acetyl | acetyl | acetyl | H | H | acetyl | acetyl | acetyl | H | H |
| 3943 | H | acetyl | acetyl | acetyl | H | H | acetyl | acetyl | acetyl | H | acetyl |
| 3944 | H | acetyl | acetyl | acetyl | H | H | acetyl | acetyl | acetyl | acetyl | H |
| 3945 | H | acetyl | acetyl | acetyl | H | H | acetyl | acetyl | acetyl | acetyl | acetyl |
| 3946 | H | acetyl | acetyl | acetyl | H | acetyl | H | H | H | H | H |
| 3947 | H | acetyl | acetyl | acetyl | H | acetyl | H | H | H | H | acetyl |
| 3948 | H | acetyl | acetyl | acetyl | H | acetyl | H | H | H | acetyl | H |
| 3949 | H | acetyl | acetyl | acetyl | H | acetyl | H | H | H | acetyl | acetyl |
| 3950 | H | acetyl | acetyl | acetyl | H | acetyl | H | H | acetyl | H | H |
| 3951 | H | acetyl | acetyl | acetyl | H | acetyl | H | H | acetyl | H | acetyl |
| 3952 | H | acetyl | acetyl | acetyl | H | acetyl | H | H | acetyl | acetyl | H |
| 3953 | H | acetyl | acetyl | acetyl | H | acetyl | H | H | acetyl | acetyl | acetyl |
| 3954 | H | acetyl | acetyl | acetyl | H | acetyl | H | acetyl | H | H | H |
| 3955 | H | acetyl | acetyl | acetyl | H | acetyl | H | acetyl | H | H | acetyl |
| 3956 | H | acetyl | acetyl | acetyl | H | acetyl | H | acetyl | H | acetyl | H |
| 3957 | H | acetyl | acetyl | acetyl | H | acetyl | H | acetyl | H | acetyl | acetyl |
| 3958 | H | acetyl | acetyl | acetyl | H | acetyl | H | acetyl | acetyl | H | H |
| 3959 | H | acetyl | acetyl | acetyl | H | acetyl | H | acetyl | acetyl | H | acetyl |
| 3960 | H | acetyl | acetyl | acetyl | H | acetyl | H | acetyl | acetyl | acetyl | H |
| 3961 | H | acetyl | acetyl | acetyl | H | acetyl | H | acetyl | acetyl | acetyl | acetyl |
| 3962 | H | acetyl | acetyl | acetyl | H | acetyl | acetyl | H | H | H | H |
| 3963 | H | acetyl | acetyl | acetyl | H | acetyl | acetyl | H | H | H | acetyl |
| 3964 | H | acetyl | acetyl | acetyl | H | acetyl | acetyl | H | H | acetyl | H |
| 3965 | H | acetyl | acetyl | acetyl | H | acetyl | acetyl | H | H | acetyl | acetyl |
| 3966 | H | acetyl | acetyl | acetyl | H | acetyl | acetyl | H | acetyl | H | H |
| 3967 | H | acetyl | acetyl | acetyl | H | acetyl | acetyl | H | acetyl | H | acetyl |
| 3968 | H | acetyl | acetyl | acetyl | H | acetyl | acetyl | H | acetyl | acetyl | H |
| 3969 | H | acetyl | acetyl | acetyl | H | acetyl | acetyl | H | acetyl | acetyl | acetyl |
| 3970 | H | acetyl | acetyl | acetyl | H | acetyl | acetyl | acetyl | H | H | H |
| 3971 | H | acetyl | acetyl | acetyl | H | acetyl | acetyl | acetyl | H | H | acetyl |
| 3972 | H | acetyl | acetyl | acetyl | H | acetyl | acetyl | acetyl | H | acetyl | H |
| 3973 | H | acetyl | acetyl | acetyl | H | acetyl | acetyl | acetyl | H | acetyl | acetyl |
| 3974 | H | acetyl | acetyl | acetyl | H | acetyl | acetyl | acetyl | acetyl | H | H |
| 3975 | H | acetyl | acetyl | acetyl | H | acetyl | acetyl | acetyl | acetyl | H | acetyl |
| 3976 | H | acetyl | acetyl | acetyl | H | acetyl | acetyl | acetyl | acetyl | acetyl | H |
| 3977 | H | acetyl | acetyl | acetyl | H | acetyl | acetyl | acetyl | acetyl | acetyl | acetyl |
| 3978 | H | acetyl | acetyl | acetyl | acetyl | H | H | H | H | H | H |
| 3979 | H | acetyl | acetyl | acetyl | acetyl | H | H | H | H | H | acetyl |
| 3980 | H | acetyl | acetyl | acetyl | acetyl | H | H | H | H | acetyl | H |
| 3981 | H | acetyl | acetyl | acetyl | acetyl | H | H | H | H | acetyl | acetyl |
| 3982 | H | acetyl | acetyl | acetyl | acetyl | H | H | H | acetyl | H | H |
| 3983 | H | acetyl | acetyl | acetyl | acetyl | H | H | H | acetyl | H | acetyl |
| 3984 | H | acetyl | acetyl | acetyl | acetyl | H | H | H | acetyl | acetyl | H |
| 3985 | H | acetyl | acetyl | acetyl | acetyl | H | H | H | acetyl | acetyl | acetyl |
| 3986 | H | acetyl | acetyl | acetyl | acetyl | H | H | acetyl | H | H | H |
| 3987 | H | acetyl | acetyl | acetyl | acetyl | H | H | acetyl | H | H | acetyl |
| 3988 | H | acetyl | acetyl | acetyl | acetyl | H | H | acetyl | H | acetyl | H |
| 3989 | H | acetyl | acetyl | acetyl | acetyl | H | H | acetyl | H | acetyl | acetyl |
| 3990 | H | acetyl | acetyl | acetyl | acetyl | H | H | acetyl | acetyl | H | H |
| 3991 | H | acetyl | acetyl | acetyl | acetyl | H | H | acetyl | acetyl | H | acetyl |
| 3992 | H | acetyl | acetyl | acetyl | acetyl | H | H | acetyl | acetyl | acetyl | H |
| 3993 | H | acetyl | acetyl | acetyl | acetyl | H | H | acetyl | acetyl | acetyl | acetyl |
| 3994 | H | acetyl | acetyl | acetyl | acetyl | H | acetyl | H | H | H | H |
| 3995 | H | acetyl | acetyl | acetyl | acetyl | H | acetyl | H | H | H | acetyl |
| 3996 | H | acetyl | acetyl | acetyl | acetyl | H | acetyl | H | H | acetyl | H |
| 3997 | H | acetyl | acetyl | acetyl | acetyl | H | acetyl | H | H | acetyl | acetyl |
| 3998 | H | acetyl | acetyl | acetyl | acetyl | H | acetyl | H | acetyl | H | H |
| 3999 | H | acetyl | acetyl | acetyl | acetyl | H | acetyl | H | acetyl | H | acetyl |
| 4000 | H | acetyl | acetyl | acetyl | acetyl | H | acetyl | H | acetyl | acetyl | H |
| 4001 | H | acetyl | acetyl | acetyl | acetyl | H | acetyl | H | acetyl | acetyl | acetyl |
| 4002 | H | acetyl | acetyl | acetyl | acetyl | H | acetyl | acetyl | H | H | H |
| 4003 | H | acetyl | acetyl | acetyl | acetyl | H | acetyl | acetyl | H | H | acetyl |
| 4004 | H | acetyl | acetyl | acetyl | acetyl | H | acetyl | acetyl | H | acetyl | H |
| 4005 | H | acetyl | acetyl | acetyl | acetyl | H | acetyl | acetyl | H | acetyl | acetyl |
| 4006 | H | acetyl | acetyl | acetyl | acetyl | H | acetyl | acetyl | acetyl | H | H |
| 4007 | H | acetyl | acetyl | acetyl | acetyl | H | acetyl | acetyl | acetyl | H | acetyl |
| 4008 | H | acetyl | acetyl | acetyl | acetyl | H | acetyl | acetyl | acetyl | acetyl | H |
| 4009 | H | acetyl | acetyl | acetyl | acetyl | H | acetyl | acetyl | acetyl | acetyl | acetyl |
| 4010 | H | acetyl | acetyl | acetyl | acetyl | acetyl | H | H | H | H | H |
| 4011 | H | acetyl | acetyl | acetyl | acetyl | acetyl | H | H | H | H | acetyl |
| 4012 | H | acetyl | acetyl | acetyl | acetyl | acetyl | H | H | H | acetyl | H |
| 4013 | H | acetyl | acetyl | acetyl | acetyl | acetyl | H | H | H | acetyl | acetyl |
| 4014 | H | acetyl | acetyl | acetyl | acetyl | acetyl | H | H | acetyl | H | H |
| 4015 | H | acetyl | acetyl | acetyl | acetyl | acetyl | H | H | acetyl | H | acetyl |
| 4016 | H | acetyl | acetyl | acetyl | acetyl | acetyl | H | H | acetyl | acetyl | H |
| 4017 | H | acetyl | acetyl | acetyl | acetyl | acetyl | H | H | acetyl | acetyl | acetyl |
| 4018 | H | acetyl | acetyl | acetyl | acetyl | acetyl | H | acetyl | H | H | H |
| 4019 | H | acetyl | acetyl | acetyl | acetyl | acetyl | H | acetyl | H | H | acetyl |
| 4020 | H | acetyl | acetyl | acetyl | acetyl | acetyl | H | acetyl | H | acetyl | H |
| 4021 | H | acetyl | acetyl | acetyl | acetyl | acetyl | H | acetyl | H | acetyl | acetyl |
| 4022 | H | acetyl | acetyl | acetyl | acetyl | acetyl | H | acetyl | acetyl | H | H |
| 4023 | H | acetyl | acetyl | acetyl | acetyl | acetyl | H | acetyl | acetyl | H | acetyl |
| 4024 | H | acetyl | acetyl | acetyl | acetyl | acetyl | H | acetyl | acetyl | acetyl | H |
| 4025 | H | acetyl | acetyl | acetyl | acetyl | acetyl | H | acetyl | acetyl | acetyl | acetyl |
| 4026 | H | acetyl | acetyl | acetyl | acetyl | acetyl | acetyl | H | H | H | H |
| 4027 | H | acetyl | acetyl | acetyl | acetyl | acetyl | acetyl | H | H | H | acetyl |
| 4028 | H | acetyl | acetyl | acetyl | acetyl | acetyl | acetyl | H | H | acetyl | H |
| 4029 | H | acetyl | acetyl | acetyl | acetyl | acetyl | acetyl | H | H | acetyl | acetyl |
| 4030 | H | acetyl | acetyl | acetyl | acetyl | acetyl | acetyl | H | acetyl | H | H |
| 4031 | H | acetyl | acetyl | acetyl | acetyl | acetyl | acetyl | H | acetyl | H | acetyl |
| 4032 | H | acetyl | acetyl | acetyl | acetyl | acetyl | acetyl | H | acetyl | acetyl | H |
| 4033 | H | acetyl | acetyl | acetyl | acetyl | acetyl | acetyl | H | acetyl | acetyl | acetyl |
| 4034 | H | acetyl | acetyl | acetyl | acetyl | acetyl | acetyl | acetyl | H | H | H |
| 4035 | H | acetyl | acetyl | acetyl | acetyl | acetyl | acetyl | acetyl | H | H | acetyl |
| 4036 | H | acetyl | acetyl | acetyl | acetyl | acetyl | acetyl | acetyl | H | acetyl | H |
| 4037 | H | acetyl | acetyl | acetyl | acetyl | acetyl | acetyl | acetyl | H | acetyl | acetyl |
| 4038 | H | acetyl | acetyl | acetyl | acetyl | acetyl | acetyl | acetyl | acetyl | H | H |
| 4039 | H | acetyl | acetyl | acetyl | acetyl | acetyl | acetyl | acetyl | acetyl | H | acetyl |
| 4040 | H | acetyl | acetyl | acetyl | acetyl | acetyl | acetyl | acetyl | acetyl | acetyl | H |
| 4041 | H | acetyl | acetyl | acetyl | acetyl | acetyl | acetyl | acetyl | acetyl | acetyl | acetyl |
| 4042 | acetyl | H | H | H | H | H | H | H | H | H | H |
| 4043 | acetyl | H | H | H | H | H | H | H | H | H | acetyl |
| 4044 | acetyl | H | H | H | H | H | H | H | H | acetyl | H |
| 4045 | acetyl | H | H | H | H | H | H | H | H | acetyl | acetyl |
| 4046 | acetyl | H | H | H | H | H | H | H | acetyl | H | H |
| 4047 | acetyl | H | H | H | H | H | H | H | acetyl | H | acetyl |
| 4048 | acetyl | H | H | H | H | H | H | H | acetyl | acetyl | H |
| 4049 | acetyl | H | H | H | H | H | H | H | acetyl | acetyl | acetyl |
| 4050 | acetyl | H | H | H | H | H | H | acetyl | H | H | H |
| 4051 | acetyl | H | H | H | H | H | H | acetyl | H | H | acetyl |
| 4052 | acetyl | H | H | H | H | H | H | acetyl | H | acetyl | H |
| 4053 | acetyl | H | H | H | H | H | H | acetyl | H | acetyl | acetyl |
| 4054 | acetyl | H | H | H | H | H | H | acetyl | acetyl | H | H |
| 4055 | acetyl | H | H | H | H | H | H | acetyl | acetyl | H | acetyl |
| 4056 | acetyl | H | H | H | H | H | H | acetyl | acetyl | acetyl | H |
| 4057 | acetyl | H | H | H | H | H | H | acetyl | acetyl | acetyl | acetyl |
| 4058 | acetyl | H | H | H | H | H | acetyl | H | H | H | H |
| 4059 | acetyl | H | H | H | H | H | acetyl | H | H | H | acetyl |
| 4060 | acetyl | H | H | H | H | H | acetyl | H | H | acetyl | H |
| 4061 | acetyl | H | H | H | H | H | acetyl | H | H | acetyl | acetyl |
| 4062 | acetyl | H | H | H | H | H | acetyl | H | acetyl | H | H |
| 4063 | acetyl | H | H | H | H | H | acetyl | H | acetyl | H | acetyl |
| 4064 | acetyl | H | H | H | H | H | acetyl | H | acetyl | acetyl | H |
| 4065 | acetyl | H | H | H | H | H | acetyl | H | acetyl | acetyl | acetyl |
| 4066 | acetyl | H | H | H | H | H | acetyl | acetyl | H | H | H |
| 4067 | acetyl | H | H | H | H | H | acetyl | acetyl | H | H | acetyl |
| 4068 | acetyl | H | H | H | H | H | acetyl | acetyl | H | acetyl | H |
| 4069 | acetyl | H | H | H | H | H | acetyl | acetyl | H | acetyl | acetyl |
| 4070 | acetyl | H | H | H | H | H | acetyl | acetyl | acetyl | H | H |
| 4071 | acetyl | H | H | H | H | H | acetyl | acetyl | acetyl | H | acetyl |
| 4072 | acetyl | H | H | H | H | H | acetyl | acetyl | acetyl | acetyl | H |
| 4073 | acetyl | H | H | H | H | H | acetyl | acetyl | acetyl | acetyl | acetyl |
| 4074 | acetyl | H | H | H | H | acetyl | H | H | H | H | H |
| 4075 | acetyl | H | H | H | H | acetyl | H | H | H | H | acetyl |
| 4076 | acetyl | H | H | H | H | acetyl | H | H | H | acetyl | H |
| 4077 | acetyl | H | H | H | H | acetyl | H | H | H | acetyl | acetyl |
| 4078 | acetyl | H | H | H | H | acetyl | H | H | acetyl | H | H |
| 4079 | acetyl | H | H | H | H | acetyl | H | H | acetyl | H | acetyl |
| 4080 | acetyl | H | H | H | H | acetyl | H | H | acetyl | acetyl | H |
| 4081 | acetyl | H | H | H | H | acetyl | H | H | acetyl | acetyl | acetyl |
| 4082 | acetyl | H | H | H | H | acetyl | H | acetyl | H | H | H |
| 4083 | acetyl | H | H | H | H | acetyl | H | acetyl | H | H | acetyl |
| 4084 | acetyl | H | H | H | H | acetyl | H | acetyl | H | acetyl | H |
| 4085 | acetyl | H | H | H | H | acetyl | H | acetyl | H | acetyl | acetyl |
| 4086 | acetyl | H | H | H | H | acetyl | H | acetyl | acetyl | H | H |
| 4087 | acetyl | H | H | H | H | acetyl | H | acetyl | acetyl | H | acetyl |
| 4088 | acetyl | H | H | H | H | acetyl | H | acetyl | acetyl | acetyl | H |
| 4089 | acetyl | H | H | H | H | acetyl | H | acetyl | acetyl | acetyl | acetyl |
| 4090 | acetyl | H | H | H | H | acetyl | acetyl | H | H | H | H |
| 4091 | acetyl | H | H | H | H | acetyl | acetyl | H | H | H | acetyl |
| 4092 | acetyl | H | H | H | H | acetyl | acetyl | H | H | acetyl | H |
| 4093 | acetyl | H | H | H | H | acetyl | acetyl | H | H | acetyl | acetyl |
| 4094 | acetyl | H | H | H | H | acetyl | acetyl | H | acetyl | H | H |
| 4095 | acetyl | H | H | H | H | acetyl | acetyl | H | acetyl | H | acetyl |
| 4096 | acetyl | H | H | H | H | acetyl | acetyl | H | acetyl | acetyl | H |
| 4097 | acetyl | H | H | H | H | acetyl | acetyl | H | acetyl | acetyl | acetyl |
| 4098 | acetyl | H | H | H | H | acetyl | acetyl | acetyl | H | H | H |
| 4099 | acetyl | H | H | H | H | acetyl | acetyl | acetyl | H | H | acetyl |
| 4100 | acetyl | H | H | H | H | acetyl | acetyl | acetyl | H | acetyl | H |
| 4101 | acetyl | H | H | H | H | acetyl | acetyl | acetyl | H | acetyl | acetyl |
| 4102 | acetyl | H | H | H | H | acetyl | acetyl | acetyl | acetyl | H | H |
| 4103 | acetyl | H | H | H | H | acetyl | acetyl | acetyl | acetyl | H | acetyl |
| 4104 | acetyl | H | H | H | H | acetyl | acetyl | acetyl | acetyl | acetyl | H |
| 4105 | acetyl | H | H | H | H | acetyl | acetyl | acetyl | acetyl | acetyl | acetyl |
| 4106 | acetyl | H | H | H | acetyl | H | H | H | H | H | H |
| 4107 | acetyl | H | H | H | acetyl | H | H | H | H | H | acetyl |
| 4108 | acetyl | H | H | H | acetyl | H | H | H | H | acetyl | H |
| 4109 | acetyl | H | H | H | acetyl | H | H | H | H | acetyl | acetyl |
| 4110 | acetyl | H | H | H | acetyl | H | H | H | acetyl | H | H |
| 4111 | acetyl | H | H | H | acetyl | H | H | H | acetyl | H | acetyl |
| 4112 | acetyl | H | H | H | acetyl | H | H | H | acetyl | acetyl | H |
| 4113 | acetyl | H | H | H | acetyl | H | H | H | acetyl | acetyl | acetyl |
| 4114 | acetyl | H | H | H | acetyl | H | H | acetyl | H | H | H |
| 4115 | acetyl | H | H | H | acetyl | H | H | acetyl | H | H | acetyl |
| 4116 | acetyl | H | H | H | acetyl | H | H | acetyl | H | acetyl | H |
| 4117 | acetyl | H | H | H | acetyl | H | H | acetyl | H | acetyl | acetyl |
| 4118 | acetyl | H | H | H | acetyl | H | H | acetyl | acetyl | H | H |
| 4119 | acetyl | H | H | H | acetyl | H | H | acetyl | acetyl | H | acetyl |
| 4120 | acetyl | H | H | H | acetyl | H | H | acetyl | acetyl | acetyl | H |
| 4121 | acetyl | H | H | H | acetyl | H | H | acetyl | acetyl | acetyl | acetyl |
| 4122 | acetyl | H | H | H | acetyl | H | acetyl | H | H | H | H |
| 4123 | acetyl | H | H | H | acetyl | H | acetyl | H | H | H | acetyl |
| 4124 | acetyl | H | H | H | acetyl | H | acetyl | H | H | acetyl | H |
| 4125 | acetyl | H | H | H | acetyl | H | acetyl | H | H | acetyl | acetyl |
| 4126 | acetyl | H | H | H | acetyl | H | acetyl | H | acetyl | H | H |
| 4127 | acetyl | H | H | H | acetyl | H | acetyl | H | acetyl | H | acetyl |
| 4128 | acetyl | H | H | H | acetyl | H | acetyl | H | acetyl | acetyl | H |
| 4129 | acetyl | H | H | H | acetyl | H | acetyl | H | acetyl | acetyl | acetyl |
| 4130 | acetyl | H | H | H | acetyl | H | acetyl | acetyl | H | H | H |
| 4131 | acetyl | H | H | H | acetyl | H | acetyl | acetyl | H | H | acetyl |
| 4132 | acetyl | H | H | H | acetyl | H | acetyl | acetyl | H | acetyl | H |
| 4133 | acetyl | H | H | H | acetyl | H | acetyl | acetyl | H | acetyl | acetyl |
| 4134 | acetyl | H | H | H | acetyl | H | acetyl | acetyl | acetyl | H | H |
| 4135 | acetyl | H | H | H | acetyl | H | acetyl | acetyl | acetyl | H | acetyl |
| 4136 | acetyl | H | H | H | acetyl | H | acetyl | acetyl | acetyl | acetyl | H |
| 4137 | acetyl | H | H | H | acetyl | H | acetyl | acetyl | acetyl | acetyl | acetyl |
| 4138 | acetyl | H | H | H | acetyl | acetyl | H | H | H | H | H |
| 4139 | acetyl | H | H | H | acetyl | acetyl | H | H | H | H | acetyl |
| 4140 | acetyl | H | H | H | acetyl | acetyl | H | H | H | acetyl | H |
| 4141 | acetyl | H | H | H | acetyl | acetyl | H | H | H | acetyl | acetyl |
| 4142 | acetyl | H | H | H | acetyl | acetyl | H | H | acetyl | H | H |
| 4143 | acetyl | H | H | H | acetyl | acetyl | H | H | acetyl | H | acetyl |
| 4144 | acetyl | H | H | H | acetyl | acetyl | H | H | acetyl | acetyl | H |
| 4145 | acetyl | H | H | H | acetyl | acetyl | H | H | acetyl | acetyl | acetyl |
| 4146 | acetyl | H | H | H | acetyl | acetyl | H | acetyl | H | H | H |
| 4147 | acetyl | H | H | H | acetyl | acetyl | H | acetyl | H | H | acetyl |
| 4148 | acetyl | H | H | H | acetyl | acetyl | H | acetyl | H | acetyl | H |
| 4149 | acetyl | H | H | H | acetyl | acetyl | H | acetyl | H | acetyl | acetyl |
| 4150 | acetyl | H | H | H | acetyl | acetyl | H | acetyl | acetyl | H | H |
| 4151 | acetyl | H | H | H | acetyl | acetyl | H | acetyl | acetyl | H | acetyl |
| 4152 | acetyl | H | H | H | acetyl | acetyl | H | acetyl | acetyl | acetyl | H |
| 4153 | acetyl | H | H | H | acetyl | acetyl | H | acetyl | acetyl | acetyl | acetyl |
| 4154 | acetyl | H | H | H | acetyl | acetyl | acetyl | H | H | H | H |
| 4155 | acetyl | H | H | H | acetyl | acetyl | acetyl | H | H | H | acetyl |
| 4156 | acetyl | H | H | H | acetyl | acetyl | acetyl | H | H | acetyl | H |
| 4157 | acetyl | H | H | H | acetyl | acetyl | acetyl | H | H | acetyl | acetyl |
| 4158 | acetyl | H | H | H | acetyl | acetyl | acetyl | H | acetyl | H | H |
| 4159 | acetyl | H | H | H | acetyl | acetyl | acetyl | H | acetyl | H | acetyl |
| 4160 | acetyl | H | H | H | acetyl | acetyl | acetyl | H | acetyl | acetyl | H |
| 4161 | acetyl | H | H | H | acetyl | acetyl | acetyl | H | acetyl | acetyl | acetyl |
| 4162 | acetyl | H | H | H | acetyl | acetyl | acetyl | acetyl | H | H | H |
| 4163 | acetyl | H | H | H | acetyl | acetyl | acetyl | acetyl | H | H | acetyl |
| 4164 | acetyl | H | H | H | acetyl | acetyl | acetyl | acetyl | H | acetyl | H |
| 4165 | acetyl | H | H | H | acetyl | acetyl | acetyl | acetyl | H | acetyl | acetyl |
| 4166 | acetyl | H | H | H | acetyl | acetyl | acetyl | acetyl | acetyl | H | H |
| 4167 | acetyl | H | H | H | acetyl | acetyl | acetyl | acetyl | acetyl | H | acetyl |
| 4168 | acetyl | H | H | H | acetyl | acetyl | acetyl | acetyl | acetyl | acetyl | H |
| 4169 | acetyl | H | H | H | acetyl | acetyl | acetyl | acetyl | acetyl | acetyl | acetyl |
| 4170 | acetyl | H | H | acetyl | H | H | H | H | H | H | H |
| 4171 | acetyl | H | H | acetyl | H | H | H | H | H | H | acetyl |
| 4172 | acetyl | H | H | acetyl | H | H | H | H | H | acetyl | H |
| 4173 | acetyl | H | H | acetyl | H | H | H | H | H | acetyl | acetyl |
| 4174 | acetyl | H | H | acetyl | H | H | H | H | acetyl | H | H |
| 4175 | acetyl | H | H | acetyl | H | H | H | H | acetyl | H | acetyl |
| 4176 | acetyl | H | H | acetyl | H | H | H | H | acetyl | acetyl | H |
| 4177 | acetyl | H | H | acetyl | H | H | H | H | acetyl | acetyl | acetyl |
| 4178 | acetyl | H | H | acetyl | H | H | H | acetyl | H | H | H |
| 4179 | acetyl | H | H | acetyl | H | H | H | acetyl | H | H | acetyl |
| 4180 | acetyl | H | H | acetyl | H | H | H | acetyl | H | acetyl | H |
| 4181 | acetyl | H | H | acetyl | H | H | H | acetyl | H | acetyl | acetyl |
| 4182 | acetyl | H | H | acetyl | H | H | H | acetyl | acetyl | H | H |
| 4183 | acetyl | H | H | acetyl | H | H | H | acetyl | acetyl | H | acetyl |
| 4184 | acetyl | H | H | acetyl | H | H | H | acetyl | acetyl | acetyl | H |
| 4185 | acetyl | H | H | acetyl | H | H | H | acetyl | acetyl | acetyl | acetyl |
| 4186 | acetyl | H | H | acetyl | H | H | acetyl | H | H | H | H |
| 4187 | acetyl | H | H | acetyl | H | H | acetyl | H | H | H | acetyl |
| 4188 | acetyl | H | H | acetyl | H | H | acetyl | H | H | acetyl | H |
| 4189 | acetyl | H | H | acetyl | H | H | acetyl | H | H | acetyl | acetyl |
| 4190 | acetyl | H | H | acetyl | H | H | acetyl | H | acetyl | H | H |
| 4191 | acetyl | H | H | acetyl | H | H | acetyl | H | acetyl | H | acetyl |
| 4192 | acetyl | H | H | acetyl | H | H | acetyl | H | acetyl | acetyl | H |
| 4193 | acetyl | H | H | acetyl | H | H | acetyl | H | acetyl | acetyl | acetyl |
| 4194 | acetyl | H | H | acetyl | H | H | acetyl | acetyl | H | H | H |
| 4195 | acetyl | H | H | acetyl | H | H | acetyl | acetyl | H | H | acetyl |
| 4196 | acetyl | H | H | acetyl | H | H | acetyl | acetyl | H | acetyl | H |
| 4197 | acetyl | H | H | acetyl | H | H | acetyl | acetyl | H | acetyl | acetyl |
| 4198 | acetyl | H | H | acetyl | H | H | acetyl | acetyl | acetyl | H | H |
| 4199 | acetyl | H | H | acetyl | H | H | acetyl | acetyl | acetyl | H | acetyl |
| 4200 | acetyl | H | H | acetyl | H | H | acetyl | acetyl | acetyl | acetyl | H |
| 4201 | acetyl | H | H | acetyl | H | H | acetyl | acetyl | acetyl | acetyl | acetyl |
| 4202 | acetyl | H | H | acetyl | H | acetyl | H | H | H | H | H |
| 4203 | acetyl | H | H | acetyl | H | acetyl | H | H | H | H | acetyl |
| 4204 | acetyl | H | H | acetyl | H | acetyl | H | H | H | acetyl | H |
| 4205 | acetyl | H | H | acetyl | H | acetyl | H | H | H | acetyl | acetyl |
| 4206 | acetyl | H | H | acetyl | H | acetyl | H | H | acetyl | H | H |
| 4207 | acetyl | H | H | acetyl | H | acetyl | H | H | acetyl | H | acetyl |
| 4208 | acetyl | H | H | acetyl | H | acetyl | H | H | acetyl | acetyl | H |
| 4209 | acetyl | H | H | acetyl | H | acetyl | H | H | acetyl | acetyl | acetyl |
| 4210 | acetyl | H | H | acetyl | H | acetyl | H | acetyl | H | H | H |
| 4211 | acetyl | H | H | acetyl | H | acetyl | H | acetyl | H | H | acetyl |
| 4212 | acetyl | H | H | acetyl | H | acetyl | H | acetyl | H | acetyl | H |
| 4213 | acetyl | H | H | acetyl | H | acetyl | H | acetyl | H | acetyl | acetyl |
| 4214 | acetyl | H | H | acetyl | H | acetyl | H | acetyl | acetyl | H | H |
| 4215 | acetyl | H | H | acetyl | H | acetyl | H | acetyl | acetyl | H | acetyl |
| 4216 | acetyl | H | H | acetyl | H | acetyl | H | acetyl | acetyl | acetyl | H |
| 4217 | acetyl | H | H | acetyl | H | acetyl | H | acetyl | acetyl | acetyl | acetyl |
| 4218 | acetyl | H | H | acetyl | H | acetyl | acetyl | H | H | H | H |
| 4219 | acetyl | H | H | acetyl | H | acetyl | acetyl | H | H | H | acetyl |
| 4220 | acetyl | H | H | acetyl | H | acetyl | acetyl | H | H | acetyl | H |
| 4221 | acetyl | H | H | acetyl | H | acetyl | acetyl | H | H | acetyl | acetyl |
| 4222 | acetyl | H | H | acetyl | H | acetyl | acetyl | H | acetyl | H | H |
| 4223 | acetyl | H | H | acetyl | H | acetyl | acetyl | H | acetyl | H | acetyl |
| 4224 | acetyl | H | H | acetyl | H | acetyl | acetyl | H | acetyl | acetyl | H |
| 4225 | acetyl | H | H | acetyl | H | acetyl | acetyl | H | acetyl | acetyl | acetyl |
| 4226 | acetyl | H | H | acetyl | H | acetyl | acetyl | acetyl | H | H | H |
| 4227 | acetyl | H | H | acetyl | H | acetyl | acetyl | acetyl | H | H | acetyl |
| 4228 | acetyl | H | H | acetyl | H | acetyl | acetyl | acetyl | H | acetyl | H |
| 4229 | acetyl | H | H | acetyl | H | acetyl | acetyl | acetyl | H | acetyl | acetyl |
| 4230 | acetyl | H | H | acetyl | H | acetyl | acetyl | acetyl | acetyl | H | H |
| 4231 | acetyl | H | H | acetyl | H | acetyl | acetyl | acetyl | acetyl | H | acetyl |
| 4232 | acetyl | H | H | acetyl | H | acetyl | acetyl | acetyl | acetyl | acetyl | H |
| 4233 | acetyl | H | H | acetyl | H | acetyl | acetyl | acetyl | acetyl | acetyl | acetyl |
| 4234 | acetyl | H | H | acetyl | acetyl | H | H | H | H | H | H |
| 4235 | acetyl | H | H | acetyl | acetyl | H | H | H | H | H | acetyl |
| 4236 | acetyl | H | H | acetyl | acetyl | H | H | H | H | acetyl | H |
| 4237 | acetyl | H | H | acetyl | acetyl | H | H | H | H | acetyl | acetyl |
| 4238 | acetyl | H | H | acetyl | acetyl | H | H | H | acetyl | H | H |
| 4239 | acetyl | H | H | acetyl | acetyl | H | H | H | acetyl | H | acetyl |
| 4240 | acetyl | H | H | acetyl | acetyl | H | H | H | acetyl | acetyl | H |
| 4241 | acetyl | H | H | acetyl | acetyl | H | H | H | acetyl | acetyl | acetyl |
| 4242 | acetyl | H | H | acetyl | acetyl | H | H | acetyl | H | H | H |
| 4243 | acetyl | H | H | acetyl | acetyl | H | H | acetyl | H | H | acetyl |
| 4244 | acetyl | H | H | acetyl | acetyl | H | H | acetyl | H | acetyl | H |
| 4245 | acetyl | H | H | acetyl | acetyl | H | H | acetyl | H | acetyl | acetyl |
| 4246 | acetyl | H | H | acetyl | acetyl | H | H | acetyl | acetyl | H | H |
| 4247 | acetyl | H | H | acetyl | acetyl | H | H | acetyl | acetyl | H | acetyl |
| 4248 | acetyl | H | H | acetyl | acetyl | H | H | acetyl | acetyl | acetyl | H |
| 4249 | acetyl | H | H | acetyl | acetyl | H | H | acetyl | acetyl | acetyl | acetyl |
| 4250 | acetyl | H | H | acetyl | acetyl | H | acetyl | H | H | H | H |
| 4251 | acetyl | H | H | acetyl | acetyl | H | acetyl | H | H | H | acetyl |
| 4252 | acetyl | H | H | acetyl | acetyl | H | acetyl | H | H | acetyl | H |
| 4253 | acetyl | H | H | acetyl | acetyl | H | acetyl | H | H | acetyl | acetyl |
| 4254 | acetyl | H | H | acetyl | acetyl | H | acetyl | H | acetyl | H | H |
| 4255 | acetyl | H | H | acetyl | acetyl | H | acetyl | H | acetyl | H | acetyl |
| 4256 | acetyl | H | H | acetyl | acetyl | H | acetyl | H | acetyl | acetyl | H |
| 4257 | acetyl | H | H | acetyl | acetyl | H | acetyl | H | acetyl | acetyl | acetyl |
| 4258 | acetyl | H | H | acetyl | acetyl | H | acetyl | acetyl | H | H | H |
| 4259 | acetyl | H | H | acetyl | acetyl | H | acetyl | acetyl | H | H | acetyl |
| 4260 | acetyl | H | H | acetyl | acetyl | H | acetyl | acetyl | H | acetyl | H |
| 4261 | acetyl | H | H | acetyl | acetyl | H | acetyl | acetyl | H | acetyl | acetyl |
| 4262 | acetyl | H | H | acetyl | acetyl | H | acetyl | acetyl | acetyl | H | H |
| 4263 | acetyl | H | H | acetyl | acetyl | H | acetyl | acetyl | acetyl | H | acetyl |
| 4264 | acetyl | H | H | acetyl | acetyl | H | acetyl | acetyl | acetyl | acetyl | H |
| 4265 | acetyl | H | H | acetyl | acetyl | H | acetyl | acetyl | acetyl | acetyl | acetyl |
| 4266 | acetyl | H | H | acetyl | acetyl | acetyl | H | H | H | H | H |
| 4267 | acetyl | H | H | acetyl | acetyl | acetyl | H | H | H | H | acetyl |
| 4268 | acetyl | H | H | acetyl | acetyl | acetyl | H | H | H | acetyl | H |
| 4269 | acetyl | H | H | acetyl | acetyl | acetyl | H | H | H | acetyl | acetyl |
| 4270 | acetyl | H | H | acetyl | acetyl | acetyl | H | H | acetyl | H | H |
| 4271 | acetyl | H | H | acetyl | acetyl | acetyl | H | H | acetyl | H | acetyl |
| 4272 | acetyl | H | H | acetyl | acetyl | acetyl | H | H | acetyl | acetyl | H |
| 4273 | acetyl | H | H | acetyl | acetyl | acetyl | H | H | acetyl | acetyl | acetyl |
| 4274 | acetyl | H | H | acetyl | acetyl | acetyl | H | acetyl | H | H | H |
| 4275 | acetyl | H | H | acetyl | acetyl | acetyl | H | acetyl | H | H | acetyl |
| 4276 | acetyl | H | H | acetyl | acetyl | acetyl | H | acetyl | H | acetyl | H |
| 4277 | acetyl | H | H | acetyl | acetyl | acetyl | H | acetyl | H | acetyl | acetyl |
| 4278 | acetyl | H | H | acetyl | acetyl | acetyl | H | acetyl | acetyl | H | H |
| 4279 | acetyl | H | H | acetyl | acetyl | acetyl | H | acetyl | acetyl | H | acetyl |
| 4280 | acetyl | H | H | acetyl | acetyl | acetyl | H | acetyl | acetyl | acetyl | H |
| 4281 | acetyl | H | H | acetyl | acetyl | acetyl | H | acetyl | acetyl | acetyl | acetyl |
| 4282 | acetyl | H | H | acetyl | acetyl | acetyl | acetyl | H | H | H | H |
| 4283 | acetyl | H | H | acetyl | acetyl | acetyl | acetyl | H | H | H | acetyl |
| 4284 | acetyl | H | H | acetyl | acetyl | acetyl | acetyl | H | H | acetyl | H |
| 4285 | acetyl | H | H | acetyl | acetyl | acetyl | acetyl | H | H | acetyl | acetyl |
| 4286 | acetyl | H | H | acetyl | acetyl | acetyl | acetyl | H | acetyl | H | H |
| 4287 | acetyl | H | H | acetyl | acetyl | acetyl | acetyl | H | acetyl | H | acetyl |
| 4288 | acetyl | H | H | acetyl | acetyl | acetyl | acetyl | H | acetyl | acetyl | H |
| 4289 | acetyl | H | H | acetyl | acetyl | acetyl | acetyl | H | acetyl | acetyl | acetyl |
| 4290 | acetyl | H | H | acetyl | acetyl | acetyl | acetyl | acetyl | H | H | H |
| 4291 | acetyl | H | H | acetyl | acetyl | acetyl | acetyl | acetyl | H | H | acetyl |
| 4292 | acetyl | H | H | acetyl | acetyl | acetyl | acetyl | acetyl | H | acetyl | H |
| 4293 | acetyl | H | H | acetyl | acetyl | acetyl | acetyl | acetyl | H | acetyl | acetyl |
| 4294 | acetyl | H | H | acetyl | acetyl | acetyl | acetyl | acetyl | acetyl | H | H |
| 4295 | acetyl | H | H | acetyl | acetyl | acetyl | acetyl | acetyl | acetyl | H | acetyl |
| 4296 | acetyl | H | H | acetyl | acetyl | acetyl | acetyl | acetyl | acetyl | acetyl | H |
| 4297 | acetyl | H | H | acetyl | acetyl | acetyl | acetyl | acetyl | acetyl | acetyl | acetyl |
| 4298 | acetyl | H | acetyl | H | H | H | H | H | H | H | H |
| 4299 | acetyl | H | acetyl | H | H | H | H | H | H | H | acetyl |
| 4300 | acetyl | H | acetyl | H | H | H | H | H | H | acetyl | H |
| 4301 | acetyl | H | acetyl | H | H | H | H | H | H | acetyl | acetyl |
| 4302 | acetyl | H | acetyl | H | H | H | H | H | acetyl | H | H |
| 4303 | acetyl | H | acetyl | H | H | H | H | H | acetyl | H | acetyl |
| 4304 | acetyl | H | acetyl | H | H | H | H | H | acetyl | acetyl | H |
| 4305 | acetyl | H | acetyl | H | H | H | H | H | acetyl | acetyl | acetyl |
| 4306 | acetyl | H | acetyl | H | H | H | H | acetyl | H | H | H |
| 4307 | acetyl | H | acetyl | H | H | H | H | acetyl | H | H | acetyl |
| 4308 | acetyl | H | acetyl | H | H | H | H | acetyl | H | acetyl | H |
| 4309 | acetyl | H | acetyl | H | H | H | H | acetyl | H | acetyl | acetyl |
| 4310 | acetyl | H | acetyl | H | H | H | H | acetyl | acetyl | H | H |
| 4311 | acetyl | H | acetyl | H | H | H | H | acetyl | acetyl | H | acetyl |
| 4312 | acetyl | H | acetyl | H | H | H | H | acetyl | acetyl | acetyl | H |
| 4313 | acetyl | H | acetyl | H | H | H | H | acetyl | acetyl | acetyl | acetyl |
| 4314 | acetyl | H | acetyl | H | H | H | acetyl | H | H | H | H |
| 4315 | acetyl | H | acetyl | H | H | H | acetyl | H | H | H | acetyl |
| 4316 | acetyl | H | acetyl | H | H | H | acetyl | H | H | acetyl | H |
| 4317 | acetyl | H | acetyl | H | H | H | acetyl | H | H | acetyl | acetyl |
| 4318 | acetyl | H | acetyl | H | H | H | acetyl | H | acetyl | H | H |
| 4319 | acetyl | H | acetyl | H | H | H | acetyl | H | acetyl | H | acetyl |
| 4320 | acetyl | H | acetyl | H | H | H | acetyl | H | acetyl | acetyl | H |
| 4321 | acetyl | H | acetyl | H | H | H | acetyl | H | acetyl | acetyl | acetyl |
| 4322 | acetyl | H | acetyl | H | H | H | acetyl | acetyl | H | H | H |
| 4323 | acetyl | H | acetyl | H | H | H | acetyl | acetyl | H | H | acetyl |
| 4324 | acetyl | H | acetyl | H | H | H | acetyl | acetyl | H | acetyl | H |
| 4325 | acetyl | H | acetyl | H | H | H | acetyl | acetyl | H | acetyl | acetyl |
| 4326 | acetyl | H | acetyl | H | H | H | acetyl | acetyl | acetyl | H | H |
| 4327 | acetyl | H | acetyl | H | H | H | acetyl | acetyl | acetyl | H | acetyl |
| 4328 | acetyl | H | acetyl | H | H | H | acetyl | acetyl | acetyl | acetyl | H |
| 4329 | acetyl | H | acetyl | H | H | H | acetyl | acetyl | acetyl | acetyl | acetyl |
| 4330 | acetyl | H | acetyl | H | H | acetyl | H | H | H | H | H |
| 4331 | acetyl | H | acetyl | H | H | acetyl | H | H | H | H | acetyl |
| 4332 | acetyl | H | acetyl | H | H | acetyl | H | H | H | acetyl | H |
| 4333 | acetyl | H | acetyl | H | H | acetyl | H | H | H | acetyl | acetyl |
| 4334 | acetyl | H | acetyl | H | H | acetyl | H | H | acetyl | H | H |
| 4335 | acetyl | H | acetyl | H | H | acetyl | H | H | acetyl | H | acetyl |
| 4336 | acetyl | H | acetyl | H | H | acetyl | H | H | acetyl | acetyl | H |
| 4337 | acetyl | H | acetyl | H | H | acetyl | H | H | acetyl | acetyl | acetyl |
| 4338 | acetyl | H | acetyl | H | H | acetyl | H | acetyl | H | H | H |
| 4339 | acetyl | H | acetyl | H | H | acetyl | H | acetyl | H | H | acetyl |
| 4340 | acetyl | H | acetyl | H | H | acetyl | H | acetyl | H | acetyl | H |
| 4341 | acetyl | H | acetyl | H | H | acetyl | H | acetyl | H | acetyl | acetyl |
| 4342 | acetyl | H | acetyl | H | H | acetyl | H | acetyl | acetyl | H | H |
| 4343 | acetyl | H | acetyl | H | H | acetyl | H | acetyl | acetyl | H | acetyl |
| 4344 | acetyl | H | acetyl | H | H | acetyl | H | acetyl | acetyl | acetyl | H |
| 4345 | acetyl | H | acetyl | H | H | acetyl | H | acetyl | acetyl | acetyl | acetyl |
| 4346 | acetyl | H | acetyl | H | H | acetyl | acetyl | H | H | H | H |
| 4347 | acetyl | H | acetyl | H | H | acetyl | acetyl | H | H | H | acetyl |
| 4348 | acetyl | H | acetyl | H | H | acetyl | acetyl | H | H | acetyl | H |
| 4349 | acetyl | H | acetyl | H | H | acetyl | acetyl | H | H | acetyl | acetyl |
| 4350 | acetyl | H | acetyl | H | H | acetyl | acetyl | H | acetyl | H | H |
| 4351 | acetyl | H | acetyl | H | H | acetyl | acetyl | H | acetyl | H | acetyl |
| 4352 | acetyl | H | acetyl | H | H | acetyl | acetyl | H | acetyl | acetyl | H |
| 4353 | acetyl | H | acetyl | H | H | acetyl | acetyl | H | acetyl | acetyl | acetyl |
| 4354 | acetyl | H | acetyl | H | H | acetyl | acetyl | acetyl | H | H | H |
| 4355 | acetyl | H | acetyl | H | H | acetyl | acetyl | acetyl | H | H | acetyl |
| 4356 | acetyl | H | acetyl | H | H | acetyl | acetyl | acetyl | H | acetyl | H |
| 4357 | acetyl | H | acetyl | H | H | acetyl | acetyl | acetyl | H | acetyl | acetyl |
| 4358 | acetyl | H | acetyl | H | H | acetyl | acetyl | acetyl | acetyl | H | H |
| 4359 | acetyl | H | acetyl | H | H | acetyl | acetyl | acetyl | acetyl | H | acetyl |
| 4360 | acetyl | H | acetyl | H | H | acetyl | acetyl | acetyl | acetyl | acetyl | H |
| 4361 | acetyl | H | acetyl | H | H | acetyl | acetyl | acetyl | acetyl | acetyl | acetyl |
| 4362 | acetyl | H | acetyl | H | acetyl | H | H | H | H | H | H |
| 4363 | acetyl | H | acetyl | H | acetyl | H | H | H | H | H | acetyl |
| 4364 | acetyl | H | acetyl | H | acetyl | H | H | H | H | acetyl | H |
| 4365 | acetyl | H | acetyl | H | acetyl | H | H | H | H | acetyl | acetyl |
| 4366 | acetyl | H | acetyl | H | acetyl | H | H | H | acetyl | H | H |
| 4367 | acetyl | H | acetyl | H | acetyl | H | H | H | acetyl | H | acetyl |
| 4368 | acetyl | H | acetyl | H | acetyl | H | H | H | acetyl | acetyl | H |
| 4369 | acetyl | H | acetyl | H | acetyl | H | H | H | acetyl | acetyl | acetyl |
| 4370 | acetyl | H | acetyl | H | acetyl | H | H | acetyl | H | H | H |
| 4371 | acetyl | H | acetyl | H | acetyl | H | H | acetyl | H | H | acetyl |
| 4372 | acetyl | H | acetyl | H | acetyl | H | H | acetyl | H | acetyl | H |
| 4373 | acetyl | H | acetyl | H | acetyl | H | H | acetyl | H | acetyl | acetyl |
| 4374 | acetyl | H | acetyl | H | acetyl | H | H | acetyl | acetyl | H | H |
| 4375 | acetyl | H | acetyl | H | acetyl | H | H | acetyl | acetyl | H | acetyl |
| 4376 | acetyl | H | acetyl | H | acetyl | H | H | acetyl | acetyl | acetyl | H |
| 4377 | acetyl | H | acetyl | H | acetyl | H | H | acetyl | acetyl | acetyl | acetyl |
| 4378 | acetyl | H | acetyl | H | acetyl | H | acetyl | H | H | H | H |
| 4379 | acetyl | H | acetyl | H | acetyl | H | acetyl | H | H | H | acetyl |
| 4380 | acetyl | H | acetyl | H | acetyl | H | acetyl | H | H | acetyl | H |
| 4381 | acetyl | H | acetyl | H | acetyl | H | acetyl | H | H | acetyl | acetyl |
| 4382 | acetyl | H | acetyl | H | acetyl | H | acetyl | H | acetyl | H | H |
| 4383 | acetyl | H | acetyl | H | acetyl | H | acetyl | H | acetyl | H | acetyl |
| 4384 | acetyl | H | acetyl | H | acetyl | H | acetyl | H | acetyl | acetyl | H |
| 4385 | acetyl | H | acetyl | H | acetyl | H | acetyl | H | acetyl | acetyl | acetyl |
| 4386 | acetyl | H | acetyl | H | acetyl | H | acetyl | acetyl | H | H | H |
| 4387 | acetyl | H | acetyl | H | acetyl | H | acetyl | acetyl | H | H | acetyl |
| 4388 | acetyl | H | acetyl | H | acetyl | H | acetyl | acetyl | H | acetyl | H |
| 4389 | acetyl | H | acetyl | H | acetyl | H | acetyl | acetyl | H | acetyl | acetyl |
| 4390 | acetyl | H | acetyl | H | acetyl | H | acetyl | acetyl | acetyl | H | H |
| 4391 | acetyl | H | acetyl | H | acetyl | H | acetyl | acetyl | acetyl | H | acetyl |
| 4392 | acetyl | H | acetyl | H | acetyl | H | acetyl | acetyl | acetyl | acetyl | H |
| 4393 | acetyl | H | acetyl | H | acetyl | H | acetyl | acetyl | acetyl | acetyl | acetyl |
| 4394 | acetyl | H | acetyl | H | acetyl | acetyl | H | H | H | H | H |
| 4395 | acetyl | H | acetyl | H | acetyl | acetyl | H | H | H | H | acetyl |
| 4396 | acetyl | H | acetyl | H | acetyl | acetyl | H | H | H | acetyl | H |
| 4397 | acetyl | H | acetyl | H | acetyl | acetyl | H | H | H | acetyl | acetyl |
| 4398 | acetyl | H | acetyl | H | acetyl | acetyl | H | H | acetyl | H | H |
| 4399 | acetyl | H | acetyl | H | acetyl | acetyl | H | H | acetyl | H | acetyl |
| 4400 | acetyl | H | acetyl | H | acetyl | acetyl | H | H | acetyl | acetyl | H |
| 4401 | acetyl | H | acetyl | H | acetyl | acetyl | H | H | acetyl | acetyl | acetyl |
| 4402 | acetyl | H | acetyl | H | acetyl | acetyl | H | acetyl | H | H | H |
| 4403 | acetyl | H | acetyl | H | acetyl | acetyl | H | acetyl | H | H | acetyl |
| 4404 | acetyl | H | acetyl | H | acetyl | acetyl | H | acetyl | H | acetyl | H |
| 4405 | acetyl | H | acetyl | H | acetyl | acetyl | H | acetyl | H | acetyl | acetyl |
| 4406 | acetyl | H | acetyl | H | acetyl | acetyl | H | acetyl | acetyl | H | H |
| 4407 | acetyl | H | acetyl | H | acetyl | acetyl | H | acetyl | acetyl | H | acetyl |
| 4408 | acetyl | H | acetyl | H | acetyl | acetyl | H | acetyl | acetyl | acetyl | H |
| 4409 | acetyl | H | acetyl | H | acetyl | acetyl | H | acetyl | acetyl | acetyl | acetyl |
| 4410 | acetyl | H | acetyl | H | acetyl | acetyl | acetyl | H | H | H | H |
| 4411 | acetyl | H | acetyl | H | acetyl | acetyl | acetyl | H | H | H | acetyl |
| 4412 | acetyl | H | acetyl | H | acetyl | acetyl | acetyl | H | H | acetyl | H |
| 4413 | acetyl | H | acetyl | H | acetyl | acetyl | acetyl | H | H | acetyl | acetyl |
| 4414 | acetyl | H | acetyl | H | acetyl | acetyl | acetyl | H | acetyl | H | H |
| 4415 | acetyl | H | acetyl | H | acetyl | acetyl | acetyl | H | acetyl | H | acetyl |
| 4416 | acetyl | H | acetyl | H | acetyl | acetyl | acetyl | H | acetyl | acetyl | H |
| 4417 | acetyl | H | acetyl | H | acetyl | acetyl | acetyl | H | acetyl | acetyl | acetyl |
| 4418 | acetyl | H | acetyl | H | acetyl | acetyl | acetyl | acetyl | H | H | H |
| 4419 | acetyl | H | acetyl | H | acetyl | acetyl | acetyl | acetyl | H | H | acetyl |
| 4420 | acetyl | H | acetyl | H | acetyl | acetyl | acetyl | acetyl | H | acetyl | H |
| 4421 | acetyl | H | acetyl | H | acetyl | acetyl | acetyl | acetyl | H | acetyl | acetyl |
| 4422 | acetyl | H | acetyl | H | acetyl | acetyl | acetyl | acetyl | acetyl | H | H |
| 4423 | acetyl | H | acetyl | H | acetyl | acetyl | acetyl | acetyl | acetyl | H | acetyl |
| 4424 | acetyl | H | acetyl | H | acetyl | acetyl | acetyl | acetyl | acetyl | acetyl | H |
| 4425 | acetyl | H | acetyl | H | acetyl | acetyl | acetyl | acetyl | acetyl | acetyl | acetyl |
| 4426 | acetyl | H | acetyl | acetyl | H | H | H | H | H | H | H |
| 4427 | acetyl | H | acetyl | acetyl | H | H | H | H | H | H | acetyl |
| 4428 | acetyl | H | acetyl | acetyl | H | H | H | H | H | acetyl | H |
| 4429 | acetyl | H | acetyl | acetyl | H | H | H | H | H | acetyl | acetyl |
| 4430 | acetyl | H | acetyl | acetyl | H | H | H | H | acetyl | H | H |
| 4431 | acetyl | H | acetyl | acetyl | H | H | H | H | acetyl | H | acetyl |
| 4432 | acetyl | H | acetyl | acetyl | H | H | H | H | acetyl | acetyl | H |
| 4433 | acetyl | H | acetyl | acetyl | H | H | H | H | acetyl | acetyl | acetyl |
| 4434 | acetyl | H | acetyl | acetyl | H | H | H | acetyl | H | H | H |
| 4435 | acetyl | H | acetyl | acetyl | H | H | H | acetyl | H | H | acetyl |
| 4436 | acetyl | H | acetyl | acetyl | H | H | H | acetyl | H | acetyl | H |
| 4437 | acetyl | H | acetyl | acetyl | H | H | H | acetyl | H | acetyl | acetyl |
| 4438 | acetyl | H | acetyl | acetyl | H | H | H | acetyl | acetyl | H | H |
| 4439 | acetyl | H | acetyl | acetyl | H | H | H | acetyl | acetyl | H | acetyl |
| 4440 | acetyl | H | acetyl | acetyl | H | H | H | acetyl | acetyl | acetyl | H |
| 4441 | acetyl | H | acetyl | acetyl | H | H | H | acetyl | acetyl | acetyl | acetyl |
| 4442 | acetyl | H | acetyl | acetyl | H | H | acetyl | H | H | H | H |
| 4443 | acetyl | H | acetyl | acetyl | H | H | acetyl | H | H | H | acetyl |
| 4444 | acetyl | H | acetyl | acetyl | H | H | acetyl | H | H | acetyl | H |
| 4445 | acetyl | H | acetyl | acetyl | H | H | acetyl | H | H | acetyl | acetyl |
| 4446 | acetyl | H | acetyl | acetyl | H | H | acetyl | H | acetyl | H | H |
| 4447 | acetyl | H | acetyl | acetyl | H | H | acetyl | H | acetyl | H | acetyl |
| 4448 | acetyl | H | acetyl | acetyl | H | H | acetyl | H | acetyl | acetyl | H |
| 4449 | acetyl | H | acetyl | acetyl | H | H | acetyl | H | acetyl | acetyl | acetyl |
| 4450 | acetyl | H | acetyl | acetyl | H | H | acetyl | acetyl | H | H | H |
| 4451 | acetyl | H | acetyl | acetyl | H | H | acetyl | acetyl | H | H | acetyl |
| 4452 | acetyl | H | acetyl | acetyl | H | H | acetyl | acetyl | H | acetyl | H |
| 4453 | acetyl | H | acetyl | acetyl | H | H | acetyl | acetyl | H | acetyl | acetyl |
| 4454 | acetyl | H | acetyl | acetyl | H | H | acetyl | acetyl | acetyl | H | H |
| 4455 | acetyl | H | acetyl | acetyl | H | H | acetyl | acetyl | acetyl | H | acetyl |
| 4456 | acetyl | H | acetyl | acetyl | H | H | acetyl | acetyl | acetyl | acetyl | H |
| 4457 | acetyl | H | acetyl | acetyl | H | H | acetyl | acetyl | acetyl | acetyl | acetyl |
| 4458 | acetyl | H | acetyl | acetyl | H | acetyl | H | H | H | H | H |
| 4459 | acetyl | H | acetyl | acetyl | H | acetyl | H | H | H | H | acetyl |
| 4460 | acetyl | H | acetyl | acetyl | H | acetyl | H | H | H | acetyl | H |
| 4461 | acetyl | H | acetyl | acetyl | H | acetyl | H | H | H | acetyl | acetyl |
| 4462 | acetyl | H | acetyl | acetyl | H | acetyl | H | H | acetyl | H | H |
| 4463 | acetyl | H | acetyl | acetyl | H | acetyl | H | H | acetyl | H | acetyl |
| 4464 | acetyl | H | acetyl | acetyl | H | acetyl | H | H | acetyl | acetyl | H |
| 4465 | acetyl | H | acetyl | acetyl | H | acetyl | H | H | acetyl | acetyl | acetyl |
| 4466 | acetyl | H | acetyl | acetyl | H | acetyl | H | acetyl | H | H | H |
| 4467 | acetyl | H | acetyl | acetyl | H | acetyl | H | acetyl | H | H | acetyl |
| 4468 | acetyl | H | acetyl | acetyl | H | acetyl | H | acetyl | H | acetyl | H |
| 4469 | acetyl | H | acetyl | acetyl | H | acetyl | H | acetyl | H | acetyl | acetyl |
| 4470 | acetyl | H | acetyl | acetyl | H | acetyl | H | acetyl | acetyl | H | H |
| 4471 | acetyl | H | acetyl | acetyl | H | acetyl | H | acetyl | acetyl | H | acetyl |
| 4472 | acetyl | H | acetyl | acetyl | H | acetyl | H | acetyl | acetyl | acetyl | H |
| 4473 | acetyl | H | acetyl | acetyl | H | acetyl | H | acetyl | acetyl | acetyl | acetyl |
| 4474 | acetyl | H | acetyl | acetyl | H | acetyl | acetyl | H | H | H | H |
| 4475 | acetyl | H | acetyl | acetyl | H | acetyl | acetyl | H | H | H | acetyl |
| 4476 | acetyl | H | acetyl | acetyl | H | acetyl | acetyl | H | H | acetyl | H |
| 4477 | acetyl | H | acetyl | acetyl | H | acetyl | acetyl | H | H | acetyl | acetyl |
| 4478 | acetyl | H | acetyl | acetyl | H | acetyl | acetyl | H | acetyl | H | H |
| 4479 | acetyl | H | acetyl | acetyl | H | acetyl | acetyl | H | acetyl | H | acetyl |
| 4480 | acetyl | H | acetyl | acetyl | H | acetyl | acetyl | H | acetyl | acetyl | H |
| 4481 | acetyl | H | acetyl | acetyl | H | acetyl | acetyl | H | acetyl | acetyl | acetyl |
| 4482 | acetyl | H | acetyl | acetyl | H | acetyl | acetyl | acetyl | H | H | H |
| 4483 | acetyl | H | acetyl | acetyl | H | acetyl | acetyl | acetyl | H | H | acetyl |
| 4484 | acetyl | H | acetyl | acetyl | H | acetyl | acetyl | acetyl | H | acetyl | H |
| 4485 | acetyl | H | acetyl | acetyl | H | acetyl | acetyl | acetyl | H | acetyl | acetyl |
| 4486 | acetyl | H | acetyl | acetyl | H | acetyl | acetyl | acetyl | acetyl | H | H |
| 4487 | acetyl | H | acetyl | acetyl | H | acetyl | acetyl | acetyl | acetyl | H | acetyl |
| 4488 | acetyl | H | acetyl | acetyl | H | acetyl | acetyl | acetyl | acetyl | acetyl | H |
| 4489 | acetyl | H | acetyl | acetyl | H | acetyl | acetyl | acetyl | acetyl | acetyl | acetyl |
| 4490 | acetyl | H | acetyl | acetyl | acetyl | H | H | H | H | H | H |
| 4491 | acetyl | H | acetyl | acetyl | acetyl | H | H | H | H | H | acetyl |
| 4492 | acetyl | H | acetyl | acetyl | acetyl | H | H | H | H | acetyl | H |
| 4493 | acetyl | H | acetyl | acetyl | acetyl | H | H | H | H | acetyl | acetyl |
| 4494 | acetyl | H | acetyl | acetyl | acetyl | H | H | H | acetyl | H | H |
| 4495 | acetyl | H | acetyl | acetyl | acetyl | H | H | H | acetyl | H | acetyl |
| 4496 | acetyl | H | acetyl | acetyl | acetyl | H | H | H | acetyl | acetyl | H |
| 4497 | acetyl | H | acetyl | acetyl | acetyl | H | H | H | acetyl | acetyl | acetyl |
| 4498 | acetyl | H | acetyl | acetyl | acetyl | H | H | acetyl | H | H | H |
| 4499 | acetyl | H | acetyl | acetyl | acetyl | H | H | acetyl | H | H | acetyl |
| 4500 | acetyl | H | acetyl | acetyl | acetyl | H | H | acetyl | H | acetyl | H |
| 4501 | acetyl | H | acetyl | acetyl | acetyl | H | H | acetyl | H | acetyl | acetyl |
| 4502 | acetyl | H | acetyl | acetyl | acetyl | H | H | acetyl | acetyl | H | H |
| 4503 | acetyl | H | acetyl | acetyl | acetyl | H | H | acetyl | acetyl | H | acetyl |
| 4504 | acetyl | H | acetyl | acetyl | acetyl | H | H | acetyl | acetyl | acetyl | H |
| 4505 | acetyl | H | acetyl | acetyl | acetyl | H | H | acetyl | acetyl | acetyl | acetyl |
| 4506 | acetyl | H | acetyl | acetyl | acetyl | H | acetyl | H | H | H | H |
| 4507 | acetyl | H | acetyl | acetyl | acetyl | H | acetyl | H | H | H | acetyl |
| 4508 | acetyl | H | acetyl | acetyl | acetyl | H | acetyl | H | H | acetyl | H |
| 4509 | acetyl | H | acetyl | acetyl | acetyl | H | acetyl | H | H | acetyl | acetyl |
| 4510 | acetyl | H | acetyl | acetyl | acetyl | H | acetyl | H | acetyl | H | H |
| 4511 | acetyl | H | acetyl | acetyl | acetyl | H | acetyl | H | acetyl | H | acetyl |
| 4512 | acetyl | H | acetyl | acetyl | acetyl | H | acetyl | H | acetyl | acetyl | H |
| 4513 | acetyl | H | acetyl | acetyl | acetyl | H | acetyl | H | acetyl | acetyl | acetyl |
| 4514 | acetyl | H | acetyl | acetyl | acetyl | H | acetyl | acetyl | H | H | H |
| 4515 | acetyl | H | acetyl | acetyl | acetyl | H | acetyl | acetyl | H | H | acetyl |
| 4516 | acetyl | H | acetyl | acetyl | acetyl | H | acetyl | acetyl | H | acetyl | H |
| 4517 | acetyl | H | acetyl | acetyl | acetyl | H | acetyl | acetyl | H | acetyl | acetyl |
| 4518 | acetyl | H | acetyl | acetyl | acetyl | H | acetyl | acetyl | acetyl | H | H |
| 4519 | acetyl | H | acetyl | acetyl | acetyl | H | acetyl | acetyl | acetyl | H | acetyl |
| 4520 | acetyl | H | acetyl | acetyl | acetyl | H | acetyl | acetyl | acetyl | acetyl | H |
| 4521 | acetyl | H | acetyl | acetyl | acetyl | H | acetyl | acetyl | acetyl | acetyl | acetyl |
| 4522 | acetyl | H | acetyl | acetyl | acetyl | acetyl | H | H | H | H | H |
| 4523 | acetyl | H | acetyl | acetyl | acetyl | acetyl | H | H | H | H | acetyl |
| 4524 | acetyl | H | acetyl | acetyl | acetyl | acetyl | H | H | H | acetyl | H |
| 4525 | acetyl | H | acetyl | acetyl | acetyl | acetyl | H | H | H | acetyl | acetyl |
| 4526 | acetyl | H | acetyl | acetyl | acetyl | acetyl | H | H | acetyl | H | H |
| 4527 | acetyl | H | acetyl | acetyl | acetyl | acetyl | H | H | acetyl | H | acetyl |
| 4528 | acetyl | H | acetyl | acetyl | acetyl | acetyl | H | H | acetyl | acetyl | H |
| 4529 | acetyl | H | acetyl | acetyl | acetyl | acetyl | H | H | acetyl | acetyl | acetyl |
| 4530 | acetyl | H | acetyl | acetyl | acetyl | acetyl | H | acetyl | H | H | H |
| 4531 | acetyl | H | acetyl | acetyl | acetyl | acetyl | H | acetyl | H | H | acetyl |
| 4532 | acetyl | H | acetyl | acetyl | acetyl | acetyl | H | acetyl | H | acetyl | H |
| 4533 | acetyl | H | acetyl | acetyl | acetyl | acetyl | H | acetyl | H | acetyl | acetyl |
| 4534 | acetyl | H | acetyl | acetyl | acetyl | acetyl | H | acetyl | acetyl | H | H |
| 4535 | acetyl | H | acetyl | acetyl | acetyl | acetyl | H | acetyl | acetyl | H | acetyl |
| 4536 | acetyl | H | acetyl | acetyl | acetyl | acetyl | H | acetyl | acetyl | acetyl | H |
| 4537 | acetyl | H | acetyl | acetyl | acetyl | acetyl | H | acetyl | acetyl | acetyl | acetyl |
| 4538 | acetyl | H | acetyl | acetyl | acetyl | acetyl | acetyl | H | H | H | H |
| 4539 | acetyl | H | acetyl | acetyl | acetyl | acetyl | acetyl | H | H | H | acetyl |
| 4540 | acetyl | H | acetyl | acetyl | acetyl | acetyl | acetyl | H | H | acetyl | H |
| 4541 | acetyl | H | acetyl | acetyl | acetyl | acetyl | acetyl | H | H | acetyl | acetyl |
| 4542 | acetyl | H | acetyl | acetyl | acetyl | acetyl | acetyl | H | acetyl | H | H |
| 4543 | acetyl | H | acetyl | acetyl | acetyl | acetyl | acetyl | H | acetyl | H | acetyl |
| 4544 | acetyl | H | acetyl | acetyl | acetyl | acetyl | acetyl | H | acetyl | acetyl | H |
| 4545 | acetyl | H | acetyl | acetyl | acetyl | acetyl | acetyl | H | acetyl | acetyl | acetyl |
| 4546 | acetyl | H | acetyl | acetyl | acetyl | acetyl | acetyl | acetyl | H | H | H |
| 4547 | acetyl | H | acetyl | acetyl | acetyl | acetyl | acetyl | acetyl | H | H | acetyl |
| 4548 | acetyl | H | acetyl | acetyl | acetyl | acetyl | acetyl | acetyl | H | acetyl | H |
| 4549 | acetyl | H | acetyl | acetyl | acetyl | acetyl | acetyl | acetyl | H | acetyl | acetyl |
| 4550 | acetyl | H | acetyl | acetyl | acetyl | acetyl | acetyl | acetyl | acetyl | H | H |
| 4551 | acetyl | H | acetyl | acetyl | acetyl | acetyl | acetyl | acetyl | acetyl | H | acetyl |
| 4552 | acetyl | H | acetyl | acetyl | acetyl | acetyl | acetyl | acetyl | acetyl | acetyl | H |
| 4553 | acetyl | H | acetyl | acetyl | acetyl | acetyl | acetyl | acetyl | acetyl | acetyl | acetyl |
| 4554 | acetyl | acetyl | H | H | H | H | H | H | H | H | H |
| 4555 | acetyl | acetyl | H | H | H | H | H | H | H | H | acetyl |
| 4556 | acetyl | acetyl | H | H | H | H | H | H | H | acetyl | H |
| 4557 | acetyl | acetyl | H | H | H | H | H | H | H | acetyl | acetyl |
| 4558 | acetyl | acetyl | H | H | H | H | H | H | acetyl | H | H |
| 4559 | acetyl | acetyl | H | H | H | H | H | H | acetyl | H | acetyl |
| 4560 | acetyl | acetyl | H | H | H | H | H | H | acetyl | acetyl | H |
| 4561 | acetyl | acetyl | H | H | H | H | H | H | acetyl | acetyl | acetyl |
| 4562 | acetyl | acetyl | H | H | H | H | H | acetyl | H | H | H |
| 4563 | acetyl | acetyl | H | H | H | H | H | acetyl | H | H | acetyl |
| 4564 | acetyl | acetyl | H | H | H | H | H | acetyl | H | acetyl | H |
| 4565 | acetyl | acetyl | H | H | H | H | H | acetyl | H | acetyl | acetyl |
| 4566 | acetyl | acetyl | H | H | H | H | H | acetyl | acetyl | H | H |
| 4567 | acetyl | acetyl | H | H | H | H | H | acetyl | acetyl | H | acetyl |
| 4568 | acetyl | acetyl | H | H | H | H | H | acetyl | acetyl | acetyl | H |
| 4569 | acetyl | acetyl | H | H | H | H | H | acetyl | acetyl | acetyl | acetyl |
| 4570 | acetyl | acetyl | H | H | H | H | acetyl | H | H | H | H |
| 4571 | acetyl | acetyl | H | H | H | H | acetyl | H | H | H | acetyl |
| 4572 | acetyl | acetyl | H | H | H | H | acetyl | H | H | acetyl | H |
| 4573 | acetyl | acetyl | H | H | H | H | acetyl | H | H | acetyl | acetyl |
| 4574 | acetyl | acetyl | H | H | H | H | acetyl | H | acetyl | H | H |
| 4575 | acetyl | acetyl | H | H | H | H | acetyl | H | acetyl | H | acetyl |
| 4576 | acetyl | acetyl | H | H | H | H | acetyl | H | acetyl | acetyl | H |
| 4577 | acetyl | acetyl | H | H | H | H | acetyl | H | acetyl | acetyl | acetyl |
| 4578 | acetyl | acetyl | H | H | H | H | acetyl | acetyl | H | H | H |
| 4579 | acetyl | acetyl | H | H | H | H | acetyl | acetyl | H | H | acetyl |
| 4580 | acetyl | acetyl | H | H | H | H | acetyl | acetyl | H | acetyl | H |
| 4581 | acetyl | acetyl | H | H | H | H | acetyl | acetyl | H | acetyl | acetyl |
| 4582 | acetyl | acetyl | H | H | H | H | acetyl | acetyl | acetyl | H | H |
| 4583 | acetyl | acetyl | H | H | H | H | acetyl | acetyl | acetyl | H | acetyl |
| 4584 | acetyl | acetyl | H | H | H | H | acetyl | acetyl | acetyl | acetyl | H |
| 4585 | acetyl | acetyl | H | H | H | H | acetyl | acetyl | acetyl | acetyl | acetyl |
| 4586 | acetyl | acetyl | H | H | H | acetyl | H | H | H | H | H |
| 4587 | acetyl | acetyl | H | H | H | acetyl | H | H | H | H | acetyl |
| 4588 | acetyl | acetyl | H | H | H | acetyl | H | H | H | acetyl | H |
| 4589 | acetyl | acetyl | H | H | H | acetyl | H | H | H | acetyl | acetyl |
| 4590 | acetyl | acetyl | H | H | H | acetyl | H | H | acetyl | H | H |
| 4591 | acetyl | acetyl | H | H | H | acetyl | H | H | acetyl | H | acetyl |
| 4592 | acetyl | acetyl | H | H | H | acetyl | H | H | acetyl | acetyl | H |
| 4593 | acetyl | acetyl | H | H | H | acetyl | H | H | acetyl | acetyl | acetyl |
| 4594 | acetyl | acetyl | H | H | H | acetyl | H | acetyl | H | H | H |
| 4595 | acetyl | acetyl | H | H | H | acetyl | H | acetyl | H | H | acetyl |
| 4596 | acetyl | acetyl | H | H | H | acetyl | H | acetyl | H | acetyl | H |
| 4597 | acetyl | acetyl | H | H | H | acetyl | H | acetyl | H | acetyl | acetyl |
| 4598 | acetyl | acetyl | H | H | H | acetyl | H | acetyl | acetyl | H | H |
| 4599 | acetyl | acetyl | H | H | H | acetyl | H | acetyl | acetyl | H | acetyl |
| 4600 | acetyl | acetyl | H | H | H | acetyl | H | acetyl | acetyl | acetyl | H |
| 4601 | acetyl | acetyl | H | H | H | acetyl | H | acetyl | acetyl | acetyl | acetyl |
| 4602 | acetyl | acetyl | H | H | H | acetyl | acetyl | H | H | H | H |
| 4603 | acetyl | acetyl | H | H | H | acetyl | acetyl | H | H | H | acetyl |
| 4604 | acetyl | acetyl | H | H | H | acetyl | acetyl | H | H | acetyl | H |
| 4605 | acetyl | acetyl | H | H | H | acetyl | acetyl | H | H | acetyl | acetyl |
| 4606 | acetyl | acetyl | H | H | H | acetyl | acetyl | H | acetyl | H | H |
| 4607 | acetyl | acetyl | H | H | H | acetyl | acetyl | H | acetyl | H | acetyl |
| 4608 | acetyl | acetyl | H | H | H | acetyl | acetyl | H | acetyl | acetyl | H |
| 4609 | acetyl | acetyl | H | H | H | acetyl | acetyl | H | acetyl | acetyl | acetyl |
| 4610 | acetyl | acetyl | H | H | H | acetyl | acetyl | acetyl | H | H | H |
| 4611 | acetyl | acetyl | H | H | H | acetyl | acetyl | acetyl | H | H | acetyl |
| 4612 | acetyl | acetyl | H | H | H | acetyl | acetyl | acetyl | H | acetyl | H |
| 4613 | acetyl | acetyl | H | H | H | acetyl | acetyl | acetyl | H | acetyl | acetyl |
| 4614 | acetyl | acetyl | H | H | H | acetyl | acetyl | acetyl | acetyl | H | H |
| 4615 | acetyl | acetyl | H | H | H | acetyl | acetyl | acetyl | acetyl | H | acetyl |
| 4616 | acetyl | acetyl | H | H | H | acetyl | acetyl | acetyl | acetyl | acetyl | H |
| 4617 | acetyl | acetyl | H | H | H | acetyl | acetyl | acetyl | acetyl | acetyl | acetyl |
| 4618 | acetyl | acetyl | H | H | acetyl | H | H | H | H | H | H |
| 4619 | acetyl | acetyl | H | H | acetyl | H | H | H | H | H | acetyl |
| 4620 | acetyl | acetyl | H | H | acetyl | H | H | H | H | acetyl | H |
| 4621 | acetyl | acetyl | H | H | acetyl | H | H | H | H | acetyl | acetyl |
| 4622 | acetyl | acetyl | H | H | acetyl | H | H | H | acetyl | H | H |
| 4623 | acetyl | acetyl | H | H | acetyl | H | H | H | acetyl | H | acetyl |
| 4624 | acetyl | acetyl | H | H | acetyl | H | H | H | acetyl | acetyl | H |
| 4625 | acetyl | acetyl | H | H | acetyl | H | H | H | acetyl | acetyl | acetyl |
| 4626 | acetyl | acetyl | H | H | acetyl | H | H | acetyl | H | H | H |
| 4627 | acetyl | acetyl | H | H | acetyl | H | H | acetyl | H | H | acetyl |
| 4628 | acetyl | acetyl | H | H | acetyl | H | H | acetyl | H | acetyl | H |
| 4629 | acetyl | acetyl | H | H | acetyl | H | H | acetyl | H | acetyl | acetyl |
| 4630 | acetyl | acetyl | H | H | acetyl | H | H | acetyl | acetyl | H | H |
| 4631 | acetyl | acetyl | H | H | acetyl | H | H | acetyl | acetyl | H | acetyl |
| 4632 | acetyl | acetyl | H | H | acetyl | H | H | acetyl | acetyl | acetyl | H |
| 4633 | acetyl | acetyl | H | H | acetyl | H | H | acetyl | acetyl | acetyl | acetyl |
| 4634 | acetyl | acetyl | H | H | acetyl | H | acetyl | H | H | H | H |
| 4635 | acetyl | acetyl | H | H | acetyl | H | acetyl | H | H | H | acetyl |
| 4636 | acetyl | acetyl | H | H | acetyl | H | acetyl | H | H | acetyl | H |
| 4637 | acetyl | acetyl | H | H | acetyl | H | acetyl | H | H | acetyl | acetyl |
| 4638 | acetyl | acetyl | H | H | acetyl | H | acetyl | H | acetyl | H | H |
| 4639 | acetyl | acetyl | H | H | acetyl | H | acetyl | H | acetyl | H | acetyl |
| 4640 | acetyl | acetyl | H | H | acetyl | H | acetyl | H | acetyl | acetyl | H |
| 4641 | acetyl | acetyl | H | H | acetyl | H | acetyl | H | acetyl | acetyl | acetyl |
| 4642 | acetyl | acetyl | H | H | acetyl | H | acetyl | acetyl | H | H | H |
| 4643 | acetyl | acetyl | H | H | acetyl | H | acetyl | acetyl | H | H | acetyl |
| 4644 | acetyl | acetyl | H | H | acetyl | H | acetyl | acetyl | H | acetyl | H |
| 4645 | acetyl | acetyl | H | H | acetyl | H | acetyl | acetyl | H | acetyl | acetyl |
| 4646 | acetyl | acetyl | H | H | acetyl | H | acetyl | acetyl | acetyl | H | H |
| 4647 | acetyl | acetyl | H | H | acetyl | H | acetyl | acetyl | acetyl | H | acetyl |
| 4648 | acetyl | acetyl | H | H | acetyl | H | acetyl | acetyl | acetyl | acetyl | H |
| 4649 | acetyl | acetyl | H | H | acetyl | H | acetyl | acetyl | acetyl | acetyl | acetyl |
| 4650 | acetyl | acetyl | H | H | acetyl | acetyl | H | H | H | H | H |
| 4651 | acetyl | acetyl | H | H | acetyl | acetyl | H | H | H | H | acetyl |
| 4652 | acetyl | acetyl | H | H | acetyl | acetyl | H | H | H | acetyl | H |
| 4653 | acetyl | acetyl | H | H | acetyl | acetyl | H | H | H | acetyl | acetyl |
| 4654 | acetyl | acetyl | H | H | acetyl | acetyl | H | H | acetyl | H | H |
| 4655 | acetyl | acetyl | H | H | acetyl | acetyl | H | H | acetyl | H | acetyl |
| 4656 | acetyl | acetyl | H | H | acetyl | acetyl | H | H | acetyl | acetyl | H |
| 4657 | acetyl | acetyl | H | H | acetyl | acetyl | H | H | acetyl | acetyl | acetyl |
| 4658 | acetyl | acetyl | H | H | acetyl | acetyl | H | acetyl | H | H | H |
| 4659 | acetyl | acetyl | H | H | acetyl | acetyl | H | acetyl | H | H | acetyl |
| 4660 | acetyl | acetyl | H | H | acetyl | acetyl | H | acetyl | H | acetyl | H |
| 4661 | acetyl | acetyl | H | H | acetyl | acetyl | H | acetyl | H | acetyl | acetyl |
| 4662 | acetyl | acetyl | H | H | acetyl | acetyl | H | acetyl | acetyl | H | H |
| 4663 | acetyl | acetyl | H | H | acetyl | acetyl | H | acetyl | acetyl | H | acetyl |
| 4664 | acetyl | acetyl | H | H | acetyl | acetyl | H | acetyl | acetyl | acetyl | H |
| 4665 | acetyl | acetyl | H | H | acetyl | acetyl | H | acetyl | acetyl | acetyl | acetyl |
| 4666 | acetyl | acetyl | H | H | acetyl | acetyl | acetyl | H | H | H | H |
| 4667 | acetyl | acetyl | H | H | acetyl | acetyl | acetyl | H | H | H | acetyl |
| 4668 | acetyl | acetyl | H | H | acetyl | acetyl | acetyl | H | H | acetyl | H |
| 4669 | acetyl | acetyl | H | H | acetyl | acetyl | acetyl | H | H | acetyl | acetyl |
| 4670 | acetyl | acetyl | H | H | acetyl | acetyl | acetyl | H | acetyl | H | H |
| 4671 | acetyl | acetyl | H | H | acetyl | acetyl | acetyl | H | acetyl | H | acetyl |
| 4672 | acetyl | acetyl | H | H | acetyl | acetyl | acetyl | H | acetyl | acetyl | H |
| 4673 | acetyl | acetyl | H | H | acetyl | acetyl | acetyl | H | acetyl | acetyl | acetyl |
| 4674 | acetyl | acetyl | H | H | acetyl | acetyl | acetyl | acetyl | H | H | H |
| 4675 | acetyl | acetyl | H | H | acetyl | acetyl | acetyl | acetyl | H | H | acetyl |
| 4676 | acetyl | acetyl | H | H | acetyl | acetyl | acetyl | acetyl | H | acetyl | H |
| 4677 | acetyl | acetyl | H | H | acetyl | acetyl | acetyl | acetyl | H | acetyl | acetyl |
| 4678 | acetyl | acetyl | H | H | acetyl | acetyl | acetyl | acetyl | acetyl | H | H |
| 4679 | acetyl | acetyl | H | H | acetyl | acetyl | acetyl | acetyl | acetyl | H | acetyl |
| 4680 | acetyl | acetyl | H | H | acetyl | acetyl | acetyl | acetyl | acetyl | acetyl | H |
| 4681 | acetyl | acetyl | H | H | acetyl | acetyl | acetyl | acetyl | acetyl | acetyl | acetyl |
| 4682 | acetyl | acetyl | H | acetyl | H | H | H | H | H | H | H |
| 4683 | acetyl | acetyl | H | acetyl | H | H | H | H | H | H | acetyl |
| 4684 | acetyl | acetyl | H | acetyl | H | H | H | H | H | acetyl | H |
| 4685 | acetyl | acetyl | H | acetyl | H | H | H | H | H | acetyl | acetyl |
| 4686 | acetyl | acetyl | H | acetyl | H | H | H | H | acetyl | H | H |
| 4687 | acetyl | acetyl | H | acetyl | H | H | H | H | acetyl | H | acetyl |
| 4688 | acetyl | acetyl | H | acetyl | H | H | H | H | acetyl | acetyl | H |
| 4689 | acetyl | acetyl | H | acetyl | H | H | H | H | acetyl | acetyl | acetyl |
| 4690 | acetyl | acetyl | H | acetyl | H | H | H | acetyl | H | H | H |
| 4691 | acetyl | acetyl | H | acetyl | H | H | H | acetyl | H | H | acetyl |
| 4692 | acetyl | acetyl | H | acetyl | H | H | H | acetyl | H | acetyl | H |
| 4693 | acetyl | acetyl | H | acetyl | H | H | H | acetyl | H | acetyl | acetyl |
| 4694 | acetyl | acetyl | H | acetyl | H | H | H | acetyl | acetyl | H | H |
| 4695 | acetyl | acetyl | H | acetyl | H | H | H | acetyl | acetyl | H | acetyl |
| 4696 | acetyl | acetyl | H | acetyl | H | H | H | acetyl | acetyl | acetyl | H |
| 4697 | acetyl | acetyl | H | acetyl | H | H | H | acetyl | acetyl | acetyl | acetyl |
| 4698 | acetyl | acetyl | H | acetyl | H | H | acetyl | H | H | H | H |
| 4699 | acetyl | acetyl | H | acetyl | H | H | acetyl | H | H | H | acetyl |
| 4700 | acetyl | acetyl | H | acetyl | H | H | acetyl | H | H | acetyl | H |
| 4701 | acetyl | acetyl | H | acetyl | H | H | acetyl | H | H | acetyl | acetyl |
| 4702 | acetyl | acetyl | H | acetyl | H | H | acetyl | H | acetyl | H | H |
| 4703 | acetyl | acetyl | H | acetyl | H | H | acetyl | H | acetyl | H | acetyl |
| 4704 | acetyl | acetyl | H | acetyl | H | H | acetyl | H | acetyl | acetyl | H |
| 4705 | acetyl | acetyl | H | acetyl | H | H | acetyl | H | acetyl | acetyl | acetyl |
| 4706 | acetyl | acetyl | H | acetyl | H | H | acetyl | acetyl | H | H | H |
| 4707 | acetyl | acetyl | H | acetyl | H | H | acetyl | acetyl | H | H | acetyl |
| 4708 | acetyl | acetyl | H | acetyl | H | H | acetyl | acetyl | H | acetyl | H |
| 4709 | acetyl | acetyl | H | acetyl | H | H | acetyl | acetyl | H | acetyl | acetyl |
| 4710 | acetyl | acetyl | H | acetyl | H | H | acetyl | acetyl | acetyl | H | H |
| 4711 | acetyl | acetyl | H | acetyl | H | H | acetyl | acetyl | acetyl | H | acetyl |
| 4712 | acetyl | acetyl | H | acetyl | H | H | acetyl | acetyl | acetyl | acetyl | H |
| 4713 | acetyl | acetyl | H | acetyl | H | H | acetyl | acetyl | acetyl | acetyl | acetyl |
| 4714 | acetyl | acetyl | H | acetyl | H | acetyl | H | H | H | H | H |
| 4715 | acetyl | acetyl | H | acetyl | H | acetyl | H | H | H | H | acetyl |
| 4716 | acetyl | acetyl | H | acetyl | H | acetyl | H | H | H | acetyl | H |
| 4717 | acetyl | acetyl | H | acetyl | H | acetyl | H | H | H | acetyl | acetyl |
| 4718 | acetyl | acetyl | H | acetyl | H | acetyl | H | H | acetyl | H | H |
| 4719 | acetyl | acetyl | H | acetyl | H | acetyl | H | H | acetyl | H | acetyl |
| 4720 | acetyl | acetyl | H | acetyl | H | acetyl | H | H | acetyl | acetyl | H |
| 4721 | acetyl | acetyl | H | acetyl | H | acetyl | H | H | acetyl | acetyl | acetyl |
| 4722 | acetyl | acetyl | H | acetyl | H | acetyl | H | acetyl | H | H | H |
| 4723 | acetyl | acetyl | H | acetyl | H | acetyl | H | acetyl | H | H | acetyl |
| 4724 | acetyl | acetyl | H | acetyl | H | acetyl | H | acetyl | H | acetyl | H |
| 4725 | acetyl | acetyl | H | acetyl | H | acetyl | H | acetyl | H | acetyl | acetyl |
| 4726 | acetyl | acetyl | H | acetyl | H | acetyl | H | acetyl | acetyl | H | H |
| 4727 | acetyl | acetyl | H | acetyl | H | acetyl | H | acetyl | acetyl | H | acetyl |
| 4728 | acetyl | acetyl | H | acetyl | H | acetyl | H | acetyl | acetyl | acetyl | H |
| 4729 | acetyl | acetyl | H | acetyl | H | acetyl | H | acetyl | acetyl | acetyl | acetyl |
| 4730 | acetyl | acetyl | H | acetyl | H | acetyl | acetyl | H | H | H | H |
| 4731 | acetyl | acetyl | H | acetyl | H | acetyl | acetyl | H | H | H | acetyl |
| 4732 | acetyl | acetyl | H | acetyl | H | acetyl | acetyl | H | H | acetyl | H |
| 4733 | acetyl | acetyl | H | acetyl | H | acetyl | acetyl | H | H | acetyl | acetyl |
| 4734 | acetyl | acetyl | H | acetyl | H | acetyl | acetyl | H | acetyl | H | H |
| 4735 | acetyl | acetyl | H | acetyl | H | acetyl | acetyl | H | acetyl | H | acetyl |
| 4736 | acetyl | acetyl | H | acetyl | H | acetyl | acetyl | H | acetyl | acetyl | H |
| 4737 | acetyl | acetyl | H | acetyl | H | acetyl | acetyl | H | acetyl | acetyl | acetyl |
| 4738 | acetyl | acetyl | H | acetyl | H | acetyl | acetyl | acetyl | H | H | H |
| 4739 | acetyl | acetyl | H | acetyl | H | acetyl | acetyl | acetyl | H | H | acetyl |
| 4740 | acetyl | acetyl | H | acetyl | H | acetyl | acetyl | acetyl | H | acetyl | H |
| 4741 | acetyl | acetyl | H | acetyl | H | acetyl | acetyl | acetyl | H | acetyl | acetyl |
| 4742 | acetyl | acetyl | H | acetyl | H | acetyl | acetyl | acetyl | acetyl | H | H |
| 4743 | acetyl | acetyl | H | acetyl | H | acetyl | acetyl | acetyl | acetyl | H | acetyl |
| 4744 | acetyl | acetyl | H | acetyl | H | acetyl | acetyl | acetyl | acetyl | acetyl | H |
| 4745 | acetyl | acetyl | H | acetyl | H | acetyl | acetyl | acetyl | acetyl | acetyl | acetyl |
| 4746 | acetyl | acetyl | H | acetyl | acetyl | H | H | H | H | H | H |
| 4747 | acetyl | acetyl | H | acetyl | acetyl | H | H | H | H | H | acetyl |
| 4748 | acetyl | acetyl | H | acetyl | acetyl | H | H | H | H | acetyl | H |
| 4749 | acetyl | acetyl | H | acetyl | acetyl | H | H | H | H | acetyl | acetyl |
| 4750 | acetyl | acetyl | H | acetyl | acetyl | H | H | H | acetyl | H | H |
| 4751 | acetyl | acetyl | H | acetyl | acetyl | H | H | H | acetyl | H | acetyl |
| 4752 | acetyl | acetyl | H | acetyl | acetyl | H | H | H | acetyl | acetyl | H |
| 4753 | acetyl | acetyl | H | acetyl | acetyl | H | H | H | acetyl | acetyl | acetyl |
| 4754 | acetyl | acetyl | H | acetyl | acetyl | H | H | acetyl | H | H | H |
| 4755 | acetyl | acetyl | H | acetyl | acetyl | H | H | acetyl | H | H | acetyl |
| 4756 | acetyl | acetyl | H | acetyl | acetyl | H | H | acetyl | H | acetyl | H |
| 4757 | acetyl | acetyl | H | acetyl | acetyl | H | H | acetyl | H | acetyl | acetyl |
| 4758 | acetyl | acetyl | H | acetyl | acetyl | H | H | acetyl | acetyl | H | H |
| 4759 | acetyl | acetyl | H | acetyl | acetyl | H | H | acetyl | acetyl | H | acetyl |
| 4760 | acetyl | acetyl | H | acetyl | acetyl | H | H | acetyl | acetyl | acetyl | H |
| 4761 | acetyl | acetyl | H | acetyl | acetyl | H | H | acetyl | acetyl | acetyl | acetyl |
| 4762 | acetyl | acetyl | H | acetyl | acetyl | H | acetyl | H | H | H | H |
| 4763 | acetyl | acetyl | H | acetyl | acetyl | H | acetyl | H | H | H | acetyl |
| 4764 | acetyl | acetyl | H | acetyl | acetyl | H | acetyl | H | H | acetyl | H |
| 4765 | acetyl | acetyl | H | acetyl | acetyl | H | acetyl | H | H | acetyl | acetyl |
| 4766 | acetyl | acetyl | H | acetyl | acetyl | H | acetyl | H | acetyl | H | H |
| 4767 | acetyl | acetyl | H | acetyl | acetyl | H | acetyl | H | acetyl | H | acetyl |
| 4768 | acetyl | acetyl | H | acetyl | acetyl | H | acetyl | H | acetyl | acetyl | H |
| 4769 | acetyl | acetyl | H | acetyl | acetyl | H | acetyl | H | acetyl | acetyl | acetyl |
| 4770 | acetyl | acetyl | H | acetyl | acetyl | H | acetyl | acetyl | H | H | H |
| 4771 | acetyl | acetyl | H | acetyl | acetyl | H | acetyl | acetyl | H | H | acetyl |
| 4772 | acetyl | acetyl | H | acetyl | acetyl | H | acetyl | acetyl | H | acetyl | H |
| 4773 | acetyl | acetyl | H | acetyl | acetyl | H | acetyl | acetyl | H | acetyl | acetyl |
| 4774 | acetyl | acetyl | H | acetyl | acetyl | H | acetyl | acetyl | acetyl | H | H |
| 4775 | acetyl | acetyl | H | acetyl | acetyl | H | acetyl | acetyl | acetyl | H | acetyl |
| 4776 | acetyl | acetyl | H | acetyl | acetyl | H | acetyl | acetyl | acetyl | acetyl | H |
| 4777 | acetyl | acetyl | H | acetyl | acetyl | H | acetyl | acetyl | acetyl | acetyl | acetyl |
| 4778 | acetyl | acetyl | H | acetyl | acetyl | acetyl | H | H | H | H | H |
| 4779 | acetyl | acetyl | H | acetyl | acetyl | acetyl | H | H | H | H | acetyl |
| 4780 | acetyl | acetyl | H | acetyl | acetyl | acetyl | H | H | H | acetyl | H |
| 4781 | acetyl | acetyl | H | acetyl | acetyl | acetyl | H | H | H | acetyl | acetyl |
| 4782 | acetyl | acetyl | H | acetyl | acetyl | acetyl | H | H | acetyl | H | H |
| 4783 | acetyl | acetyl | H | acetyl | acetyl | acetyl | H | H | acetyl | H | acetyl |
| 4784 | acetyl | acetyl | H | acetyl | acetyl | acetyl | H | H | acetyl | acetyl | H |
| 4785 | acetyl | acetyl | H | acetyl | acetyl | acetyl | H | H | acetyl | acetyl | acetyl |
| 4786 | acetyl | acetyl | H | acetyl | acetyl | acetyl | H | acetyl | H | H | H |
| 4787 | acetyl | acetyl | H | acetyl | acetyl | acetyl | H | acetyl | H | H | acetyl |
| 4788 | acetyl | acetyl | H | acetyl | acetyl | acetyl | H | acetyl | H | acetyl | H |
| 4789 | acetyl | acetyl | H | acetyl | acetyl | acetyl | H | acetyl | H | acetyl | acetyl |
| 4790 | acetyl | acetyl | H | acetyl | acetyl | acetyl | H | acetyl | acetyl | H | H |
| 4791 | acetyl | acetyl | H | acetyl | acetyl | acetyl | H | acetyl | acetyl | H | acetyl |
| 4792 | acetyl | acetyl | H | acetyl | acetyl | acetyl | H | acetyl | acetyl | acetyl | H |
| 4793 | acetyl | acetyl | H | acetyl | acetyl | acetyl | H | acetyl | acetyl | acetyl | acetyl |
| 4794 | acetyl | acetyl | H | acetyl | acetyl | acetyl | acetyl | H | H | H | H |
| 4795 | acetyl | acetyl | H | acetyl | acetyl | acetyl | acetyl | H | H | H | acetyl |
| 4796 | acetyl | acetyl | H | acetyl | acetyl | acetyl | acetyl | H | H | acetyl | H |
| 4797 | acetyl | acetyl | H | acetyl | acetyl | acetyl | acetyl | H | H | acetyl | acetyl |
| 4798 | acetyl | acetyl | H | acetyl | acetyl | acetyl | acetyl | H | acetyl | H | H |
| 4799 | acetyl | acetyl | H | acetyl | acetyl | acetyl | acetyl | H | acetyl | H | acetyl |
| 4800 | acetyl | acetyl | H | acetyl | acetyl | acetyl | acetyl | H | acetyl | acetyl | H |
| 4801 | acetyl | acetyl | H | acetyl | acetyl | acetyl | acetyl | H | acetyl | acetyl | acetyl |
| 4802 | acetyl | acetyl | H | acetyl | acetyl | acetyl | acetyl | acetyl | H | H | H |
| 4803 | acetyl | acetyl | H | acetyl | acetyl | acetyl | acetyl | acetyl | H | H | acetyl |
| 4804 | acetyl | acetyl | H | acetyl | acetyl | acetyl | acetyl | acetyl | H | acetyl | H |
| 4805 | acetyl | acetyl | H | acetyl | acetyl | acetyl | acetyl | acetyl | H | acetyl | acetyl |
| 4806 | acetyl | acetyl | H | acetyl | acetyl | acetyl | acetyl | acetyl | acetyl | H | H |
| 4807 | acetyl | acetyl | H | acetyl | acetyl | acetyl | acetyl | acetyl | acetyl | H | acetyl |
| 4808 | acetyl | acetyl | H | acetyl | acetyl | acetyl | acetyl | acetyl | acetyl | acetyl | H |
| 4809 | acetyl | acetyl | H | acetyl | acetyl | acetyl | acetyl | acetyl | acetyl | acetyl | acetyl |
| 4810 | acetyl | acetyl | acetyl | H | H | H | H | H | H | H | H |
| 4811 | acetyl | acetyl | acetyl | H | H | H | H | H | H | H | acetyl |
| 4812 | acetyl | acetyl | acetyl | H | H | H | H | H | H | acetyl | H |
| 4813 | acetyl | acetyl | acetyl | H | H | H | H | H | H | acetyl | acetyl |
| 4814 | acetyl | acetyl | acetyl | H | H | H | H | H | acetyl | H | H |
| 4815 | acetyl | acetyl | acetyl | H | H | H | H | H | acetyl | H | acetyl |
| 4816 | acetyl | acetyl | acetyl | H | H | H | H | H | acetyl | acetyl | H |
| 4817 | acetyl | acetyl | acetyl | H | H | H | H | H | acetyl | acetyl | acetyl |
| 4818 | acetyl | acetyl | acetyl | H | H | H | H | acetyl | H | H | H |
| 4819 | acetyl | acetyl | acetyl | H | H | H | H | acetyl | H | H | acetyl |
| 4820 | acetyl | acetyl | acetyl | H | H | H | H | acetyl | H | acetyl | H |
| 4821 | acetyl | acetyl | acetyl | H | H | H | H | acetyl | H | acetyl | acetyl |
| 4822 | acetyl | acetyl | acetyl | H | H | H | H | acetyl | acetyl | H | H |
| 4823 | acetyl | acetyl | acetyl | H | H | H | H | acetyl | acetyl | H | acetyl |
| 4824 | acetyl | acetyl | acetyl | H | H | H | H | acetyl | acetyl | acetyl | H |
| 4825 | acetyl | acetyl | acetyl | H | H | H | H | acetyl | acetyl | acetyl | acetyl |
| 4826 | acetyl | acetyl | acetyl | H | H | H | acetyl | H | H | H | H |
| 4827 | acetyl | acetyl | acetyl | H | H | H | acetyl | H | H | H | acetyl |
| 4828 | acetyl | acetyl | acetyl | H | H | H | acetyl | H | H | acetyl | H |
| 4829 | acetyl | acetyl | acetyl | H | H | H | acetyl | H | H | acetyl | acetyl |
| 4830 | acetyl | acetyl | acetyl | H | H | H | acetyl | H | acetyl | H | H |
| 4831 | acetyl | acetyl | acetyl | H | H | H | acetyl | H | acetyl | H | acetyl |
| 4832 | acetyl | acetyl | acetyl | H | H | H | acetyl | H | acetyl | acetyl | H |
| 4833 | acetyl | acetyl | acetyl | H | H | H | acetyl | H | acetyl | acetyl | acetyl |
| 4834 | acetyl | acetyl | acetyl | H | H | H | acetyl | acetyl | H | H | H |
| 4835 | acetyl | acetyl | acetyl | H | H | H | acetyl | acetyl | H | H | acetyl |
| 4836 | acetyl | acetyl | acetyl | H | H | H | acetyl | acetyl | H | acetyl | H |
| 4837 | acetyl | acetyl | acetyl | H | H | H | acetyl | acetyl | H | acetyl | acetyl |
| 4838 | acetyl | acetyl | acetyl | H | H | H | acetyl | acetyl | acetyl | H | H |
| 4839 | acetyl | acetyl | acetyl | H | H | H | acetyl | acetyl | acetyl | H | acetyl |
| 4840 | acetyl | acetyl | acetyl | H | H | H | acetyl | acetyl | acetyl | acetyl | H |
| 4841 | acetyl | acetyl | acetyl | H | H | H | acetyl | acetyl | acetyl | acetyl | acetyl |
| 4842 | acetyl | acetyl | acetyl | H | H | acetyl | H | H | H | H | H |
| 4843 | acetyl | acetyl | acetyl | H | H | acetyl | H | H | H | H | acetyl |
| 4844 | acetyl | acetyl | acetyl | H | H | acetyl | H | H | H | acetyl | H |
| 4845 | acetyl | acetyl | acetyl | H | H | acetyl | H | H | H | acetyl | acetyl |
| 4846 | acetyl | acetyl | acetyl | H | H | acetyl | H | H | acetyl | H | H |
| 4847 | acetyl | acetyl | acetyl | H | H | acetyl | H | H | acetyl | H | acetyl |
| 4848 | acetyl | acetyl | acetyl | H | H | acetyl | H | H | acetyl | acetyl | H |
| 4849 | acetyl | acetyl | acetyl | H | H | acetyl | H | H | acetyl | acetyl | acetyl |
| 4850 | acetyl | acetyl | acetyl | H | H | acetyl | H | acetyl | H | H | H |
| 4851 | acetyl | acetyl | acetyl | H | H | acetyl | H | acetyl | H | H | acetyl |
| 4852 | acetyl | acetyl | acetyl | H | H | acetyl | H | acetyl | H | acetyl | H |
| 4853 | acetyl | acetyl | acetyl | H | H | acetyl | H | acetyl | H | acetyl | acetyl |
| 4854 | acetyl | acetyl | acetyl | H | H | acetyl | H | acetyl | acetyl | H | H |
| 4855 | acetyl | acetyl | acetyl | H | H | acetyl | H | acetyl | acetyl | H | acetyl |
| 4856 | acetyl | acetyl | acetyl | H | H | acetyl | H | acetyl | acetyl | acetyl | H |
| 4857 | acetyl | acetyl | acetyl | H | H | acetyl | H | acetyl | acetyl | acetyl | acetyl |
| 4858 | acetyl | acetyl | acetyl | H | H | acetyl | acetyl | H | H | H | H |
| 4859 | acetyl | acetyl | acetyl | H | H | acetyl | acetyl | H | H | H | acetyl |
| 4860 | acetyl | acetyl | acetyl | H | H | acetyl | acetyl | H | H | acetyl | H |
| 4861 | acetyl | acetyl | acetyl | H | H | acetyl | acetyl | H | H | acetyl | acetyl |
| 4862 | acetyl | acetyl | acetyl | H | H | acetyl | acetyl | H | acetyl | H | H |
| 4863 | acetyl | acetyl | acetyl | H | H | acetyl | acetyl | H | acetyl | H | acetyl |
| 4864 | acetyl | acetyl | acetyl | H | H | acetyl | acetyl | H | acetyl | acetyl | H |
| 4865 | acetyl | acetyl | acetyl | H | H | acetyl | acetyl | H | acetyl | acetyl | acetyl |
| 4866 | acetyl | acetyl | acetyl | H | H | acetyl | acetyl | acetyl | H | H | H |
| 4867 | acetyl | acetyl | acetyl | H | H | acetyl | acetyl | acetyl | H | H | acetyl |
| 4868 | acetyl | acetyl | acetyl | H | H | acetyl | acetyl | acetyl | H | acetyl | H |
| 4869 | acetyl | acetyl | acetyl | H | H | acetyl | acetyl | acetyl | H | acetyl | acetyl |
| 4870 | acetyl | acetyl | acetyl | H | H | acetyl | acetyl | acetyl | acetyl | H | H |
| 4871 | acetyl | acetyl | acetyl | H | H | acetyl | acetyl | acetyl | acetyl | H | acetyl |
| 4872 | acetyl | acetyl | acetyl | H | H | acetyl | acetyl | acetyl | acetyl | acetyl | H |
| 4873 | acetyl | acetyl | acetyl | H | H | acetyl | acetyl | acetyl | acetyl | acetyl | acetyl |
| 4874 | acetyl | acetyl | acetyl | H | acetyl | H | H | H | H | H | H |
| 4875 | acetyl | acetyl | acetyl | H | acetyl | H | H | H | H | H | acetyl |
| 4876 | acetyl | acetyl | acetyl | H | acetyl | H | H | H | H | acetyl | H |
| 4877 | acetyl | acetyl | acetyl | H | acetyl | H | H | H | H | acetyl | acetyl |
| 4878 | acetyl | acetyl | acetyl | H | acetyl | H | H | H | acetyl | H | H |
| 4879 | acetyl | acetyl | acetyl | H | acetyl | H | H | H | acetyl | H | acetyl |
| 4880 | acetyl | acetyl | acetyl | H | acetyl | H | H | H | acetyl | acetyl | H |
| 4881 | acetyl | acetyl | acetyl | H | acetyl | H | H | H | acetyl | acetyl | acetyl |
| 4882 | acetyl | acetyl | acetyl | H | acetyl | H | H | acetyl | H | H | H |
| 4883 | acetyl | acetyl | acetyl | H | acetyl | H | H | acetyl | H | H | acetyl |
| 4884 | acetyl | acetyl | acetyl | H | acetyl | H | H | acetyl | H | acetyl | H |
| 4885 | acetyl | acetyl | acetyl | H | acetyl | H | H | acetyl | H | acetyl | acetyl |
| 4886 | acetyl | acetyl | acetyl | H | acetyl | H | H | acetyl | acetyl | H | H |
| 4887 | acetyl | acetyl | acetyl | H | acetyl | H | H | acetyl | acetyl | H | acetyl |
| 4888 | acetyl | acetyl | acetyl | H | acetyl | H | H | acetyl | acetyl | acetyl | H |
| 4889 | acetyl | acetyl | acetyl | H | acetyl | H | H | acetyl | acetyl | acetyl | acetyl |
| 4890 | acetyl | acetyl | acetyl | H | acetyl | H | acetyl | H | H | H | H |
| 4891 | acetyl | acetyl | acetyl | H | acetyl | H | acetyl | H | H | H | acetyl |
| 4892 | acetyl | acetyl | acetyl | H | acetyl | H | acetyl | H | H | acetyl | H |
| 4893 | acetyl | acetyl | acetyl | H | acetyl | H | acetyl | H | H | acetyl | acetyl |
| 4894 | acetyl | acetyl | acetyl | H | acetyl | H | acetyl | H | acetyl | H | H |
| 4895 | acetyl | acetyl | acetyl | H | acetyl | H | acetyl | H | acetyl | H | acetyl |
| 4896 | acetyl | acetyl | acetyl | H | acetyl | H | acetyl | H | acetyl | acetyl | H |
| 4897 | acetyl | acetyl | acetyl | H | acetyl | H | acetyl | H | acetyl | acetyl | acetyl |
| 4898 | acetyl | acetyl | acetyl | H | acetyl | H | acetyl | acetyl | H | H | H |
| 4899 | acetyl | acetyl | acetyl | H | acetyl | H | acetyl | acetyl | H | H | acetyl |
| 4900 | acetyl | acetyl | acetyl | H | acetyl | H | acetyl | acetyl | H | acetyl | H |
| 4901 | acetyl | acetyl | acetyl | H | acetyl | H | acetyl | acetyl | H | acetyl | acetyl |
| 4902 | acetyl | acetyl | acetyl | H | acetyl | H | acetyl | acetyl | acetyl | H | H |
| 4903 | acetyl | acetyl | acetyl | H | acetyl | H | acetyl | acetyl | acetyl | H | acetyl |
| 4904 | acetyl | acetyl | acetyl | H | acetyl | H | acetyl | acetyl | acetyl | acetyl | H |
| 4905 | acetyl | acetyl | acetyl | H | acetyl | H | acetyl | acetyl | acetyl | acetyl | acetyl |
| 4906 | acetyl | acetyl | acetyl | H | acetyl | acetyl | H | H | H | H | H |
| 4907 | acetyl | acetyl | acetyl | H | acetyl | acetyl | H | H | H | H | acetyl |
| 4908 | acetyl | acetyl | acetyl | H | acetyl | acetyl | H | H | H | acetyl | H |
| 4909 | acetyl | acetyl | acetyl | H | acetyl | acetyl | H | H | H | acetyl | acetyl |
| 4910 | acetyl | acetyl | acetyl | H | acetyl | acetyl | H | H | acetyl | H | H |
| 4911 | acetyl | acetyl | acetyl | H | acetyl | acetyl | H | H | acetyl | H | acetyl |
| 4912 | acetyl | acetyl | acetyl | H | acetyl | acetyl | H | H | acetyl | acetyl | H |
| 4913 | acetyl | acetyl | acetyl | H | acetyl | acetyl | H | H | acetyl | acetyl | acetyl |
| 4914 | acetyl | acetyl | acetyl | H | acetyl | acetyl | H | acetyl | H | H | H |
| 4915 | acetyl | acetyl | acetyl | H | acetyl | acetyl | H | acetyl | H | H | acetyl |
| 4916 | acetyl | acetyl | acetyl | H | acetyl | acetyl | H | acetyl | H | acetyl | H |
| 4917 | acetyl | acetyl | acetyl | H | acetyl | acetyl | H | acetyl | H | acetyl | acetyl |
| 4918 | acetyl | acetyl | acetyl | H | acetyl | acetyl | H | acetyl | acetyl | H | H |
| 4919 | acetyl | acetyl | acetyl | H | acetyl | acetyl | H | acetyl | acetyl | H | acetyl |
| 4920 | acetyl | acetyl | acetyl | H | acetyl | acetyl | H | acetyl | acetyl | acetyl | H |
| 4921 | acetyl | acetyl | acetyl | H | acetyl | acetyl | H | acetyl | acetyl | acetyl | acetyl |
| 4922 | acetyl | acetyl | acetyl | H | acetyl | acetyl | acetyl | H | H | H | H |
| 4923 | acetyl | acetyl | acetyl | H | acetyl | acetyl | acetyl | H | H | H | acetyl |
| 4924 | acetyl | acetyl | acetyl | H | acetyl | acetyl | acetyl | H | H | acetyl | H |
| 4925 | acetyl | acetyl | acetyl | H | acetyl | acetyl | acetyl | H | H | acetyl | acetyl |
| 4926 | acetyl | acetyl | acetyl | H | acetyl | acetyl | acetyl | H | acetyl | H | H |
| 4927 | acetyl | acetyl | acetyl | H | acetyl | acetyl | acetyl | H | acetyl | H | acetyl |
| 4928 | acetyl | acetyl | acetyl | H | acetyl | acetyl | acetyl | H | acetyl | acetyl | H |
| 4929 | acetyl | acetyl | acetyl | H | acetyl | acetyl | acetyl | H | acetyl | acetyl | acetyl |
| 4930 | acetyl | acetyl | acetyl | H | acetyl | acetyl | acetyl | acetyl | H | H | H |
| 4931 | acetyl | acetyl | acetyl | H | acetyl | acetyl | acetyl | acetyl | H | H | acetyl |
| 4932 | acetyl | acetyl | acetyl | H | acetyl | acetyl | acetyl | acetyl | H | acetyl | H |
| 4933 | acetyl | acetyl | acetyl | H | acetyl | acetyl | acetyl | acetyl | H | acetyl | acetyl |
| 4934 | acetyl | acetyl | acetyl | H | acetyl | acetyl | acetyl | acetyl | acetyl | H | H |
| 4935 | acetyl | acetyl | acetyl | H | acetyl | acetyl | acetyl | acetyl | acetyl | H | acetyl |
| 4936 | acetyl | acetyl | acetyl | H | acetyl | acetyl | acetyl | acetyl | acetyl | acetyl | H |
| 4937 | acetyl | acetyl | acetyl | H | acetyl | acetyl | acetyl | acetyl | acetyl | acetyl | acetyl |
| 4938 | acetyl | acetyl | acetyl | acetyl | H | H | H | H | H | H | H |
| 4939 | acetyl | acetyl | acetyl | acetyl | H | H | H | H | H | H | acetyl |
| 4940 | acetyl | acetyl | acetyl | acetyl | H | H | H | H | H | acetyl | H |
| 4941 | acetyl | acetyl | acetyl | acetyl | H | H | H | H | H | acetyl | acetyl |
| 4942 | acetyl | acetyl | acetyl | acetyl | H | H | H | H | acetyl | H | H |
| 4943 | acetyl | acetyl | acetyl | acetyl | H | H | H | H | acetyl | H | acetyl |
| 4944 | acetyl | acetyl | acetyl | acetyl | H | H | H | H | acetyl | acetyl | H |
| 4945 | acetyl | acetyl | acetyl | acetyl | H | H | H | H | acetyl | acetyl | acetyl |
| 4946 | acetyl | acetyl | acetyl | acetyl | H | H | H | acetyl | H | H | H |
| 4947 | acetyl | acetyl | acetyl | acetyl | H | H | H | acetyl | H | H | acetyl |
| 4948 | acetyl | acetyl | acetyl | acetyl | H | H | H | acetyl | H | acetyl | H |
| 4949 | acetyl | acetyl | acetyl | acetyl | H | H | H | acetyl | H | acetyl | acetyl |
| 4950 | acetyl | acetyl | acetyl | acetyl | H | H | H | acetyl | acetyl | H | H |
| 4951 | acetyl | acetyl | acetyl | acetyl | H | H | H | acetyl | acetyl | H | acetyl |
| 4952 | acetyl | acetyl | acetyl | acetyl | H | H | H | acetyl | acetyl | acetyl | H |
| 4953 | acetyl | acetyl | acetyl | acetyl | H | H | H | acetyl | acetyl | acetyl | acetyl |
| 4954 | acetyl | acetyl | acetyl | acetyl | H | H | acetyl | H | H | H | H |
| 4955 | acetyl | acetyl | acetyl | acetyl | H | H | acetyl | H | H | H | acetyl |
| 4956 | acetyl | acetyl | acetyl | acetyl | H | H | acetyl | H | H | acetyl | H |
| 4957 | acetyl | acetyl | acetyl | acetyl | H | H | acetyl | H | H | acetyl | acetyl |
| 4958 | acetyl | acetyl | acetyl | acetyl | H | H | acetyl | H | acetyl | H | H |
| 4959 | acetyl | acetyl | acetyl | acetyl | H | H | acetyl | H | acetyl | H | acetyl |
| 4960 | acetyl | acetyl | acetyl | acetyl | H | H | acetyl | H | acetyl | acetyl | H |
| 4961 | acetyl | acetyl | acetyl | acetyl | H | H | acetyl | H | acetyl | acetyl | acetyl |
| 4962 | acetyl | acetyl | acetyl | acetyl | H | H | acetyl | acetyl | H | H | H |
| 4963 | acetyl | acetyl | acetyl | acetyl | H | H | acetyl | acetyl | H | H | acetyl |
| 4964 | acetyl | acetyl | acetyl | acetyl | H | H | acetyl | acetyl | H | acetyl | H |
| 4965 | acetyl | acetyl | acetyl | acetyl | H | H | acetyl | acetyl | H | acetyl | acetyl |
| 4966 | acetyl | acetyl | acetyl | acetyl | H | H | acetyl | acetyl | acetyl | H | H |
| 4967 | acetyl | acetyl | acetyl | acetyl | H | H | acetyl | acetyl | acetyl | H | acetyl |
| 4968 | acetyl | acetyl | acetyl | acetyl | H | H | acetyl | acetyl | acetyl | acetyl | H |
| 4969 | acetyl | acetyl | acetyl | acetyl | H | H | acetyl | acetyl | acetyl | acetyl | acetyl |
| 4970 | acetyl | acetyl | acetyl | acetyl | H | acetyl | H | H | H | H | H |
| 4971 | acetyl | acetyl | acetyl | acetyl | H | acetyl | H | H | H | H | acetyl |
| 4972 | acetyl | acetyl | acetyl | acetyl | H | acetyl | H | H | H | acetyl | H |
| 4973 | acetyl | acetyl | acetyl | acetyl | H | acetyl | H | H | H | acetyl | acetyl |
| 4974 | acetyl | acetyl | acetyl | acetyl | H | acetyl | H | H | acetyl | H | H |
| 4975 | acetyl | acetyl | acetyl | acetyl | H | acetyl | H | H | acetyl | H | acetyl |
| 4976 | acetyl | acetyl | acetyl | acetyl | H | acetyl | H | H | acetyl | acetyl | H |
| 4977 | acetyl | acetyl | acetyl | acetyl | H | acetyl | H | H | acetyl | acetyl | acetyl |
| 4978 | acetyl | acetyl | acetyl | acetyl | H | acetyl | H | acetyl | H | H | H |
| 4979 | acetyl | acetyl | acetyl | acetyl | H | acetyl | H | acetyl | H | H | acetyl |
| 4980 | acetyl | acetyl | acetyl | acetyl | H | acetyl | H | acetyl | H | acetyl | H |
| 4981 | acetyl | acetyl | acetyl | acetyl | H | acetyl | H | acetyl | H | acetyl | acetyl |
| 4982 | acetyl | acetyl | acetyl | acetyl | H | acetyl | H | acetyl | acetyl | H | H |
| 4983 | acetyl | acetyl | acetyl | acetyl | H | acetyl | H | acetyl | acetyl | H | acetyl |
| 4984 | acetyl | acetyl | acetyl | acetyl | H | acetyl | H | acetyl | acetyl | acetyl | H |
| 4985 | acetyl | acetyl | acetyl | acetyl | H | acetyl | H | acetyl | acetyl | acetyl | acetyl |
| 4986 | acetyl | acetyl | acetyl | acetyl | H | acetyl | acetyl | H | H | H | H |
| 4987 | acetyl | acetyl | acetyl | acetyl | H | acetyl | acetyl | H | H | H | acetyl |
| 4988 | acetyl | acetyl | acetyl | acetyl | H | acetyl | acetyl | H | H | acetyl | H |
| 4989 | acetyl | acetyl | acetyl | acetyl | H | acetyl | acetyl | H | H | acetyl | acetyl |
| 4990 | acetyl | acetyl | acetyl | acetyl | H | acetyl | acetyl | H | acetyl | H | H |
| 4991 | acetyl | acetyl | acetyl | acetyl | H | acetyl | acetyl | H | acetyl | H | acetyl |
| 4992 | acetyl | acetyl | acetyl | acetyl | H | acetyl | acetyl | H | acetyl | acetyl | H |
| 4993 | acetyl | acetyl | acetyl | acetyl | H | acetyl | acetyl | H | acetyl | acetyl | acetyl |
| 4994 | acetyl | acetyl | acetyl | acetyl | H | acetyl | acetyl | acetyl | H | H | H |
| 4995 | acetyl | acetyl | acetyl | acetyl | H | acetyl | acetyl | acetyl | H | H | acetyl |
| 4996 | acetyl | acetyl | acetyl | acetyl | H | acetyl | acetyl | acetyl | H | acetyl | H |
| 4997 | acetyl | acetyl | acetyl | acetyl | H | acetyl | acetyl | acetyl | H | acetyl | acetyl |
| 4998 | acetyl | acetyl | acetyl | acetyl | H | acetyl | acetyl | acetyl | acetyl | H | H |
| 4999 | acetyl | acetyl | acetyl | acetyl | H | acetyl | acetyl | acetyl | acetyl | H | acetyl |
| 5000 | acetyl | acetyl | acetyl | acetyl | H | acetyl | acetyl | acetyl | acetyl | acetyl | H |
| 5001 | acetyl | acetyl | acetyl | acetyl | H | acetyl | acetyl | acetyl | acetyl | acetyl | acetyl |
| 5002 | acetyl | acetyl | acetyl | acetyl | acetyl | H | H | H | H | H | H |
| 5003 | acetyl | acetyl | acetyl | acetyl | acetyl | H | H | H | H | H | acetyl |
| 5004 | acetyl | acetyl | acetyl | acetyl | acetyl | H | H | H | H | acetyl | H |
| 5005 | acetyl | acetyl | acetyl | acetyl | acetyl | H | H | H | H | acetyl | acetyl |
| 5006 | acetyl | acetyl | acetyl | acetyl | acetyl | H | H | H | acetyl | H | H |
| 5007 | acetyl | acetyl | acetyl | acetyl | acetyl | H | H | H | acetyl | H | acetyl |
| 5008 | acetyl | acetyl | acetyl | acetyl | acetyl | H | H | H | acetyl | acetyl | H |
| 5009 | acetyl | acetyl | acetyl | acetyl | acetyl | H | H | H | acetyl | acetyl | acetyl |
| 5010 | acetyl | acetyl | acetyl | acetyl | acetyl | H | H | acetyl | H | H | H |
| 5011 | acetyl | acetyl | acetyl | acetyl | acetyl | H | H | acetyl | H | H | acetyl |
| 5012 | acetyl | acetyl | acetyl | acetyl | acetyl | H | H | acetyl | H | acetyl | H |
| 5013 | acetyl | acetyl | acetyl | acetyl | acetyl | H | H | acetyl | H | acetyl | acetyl |
| 5014 | acetyl | acetyl | acetyl | acetyl | acetyl | H | H | acetyl | acetyl | H | H |
| 5015 | acetyl | acetyl | acetyl | acetyl | acetyl | H | H | acetyl | acetyl | H | acetyl |
| 5016 | acetyl | acetyl | acetyl | acetyl | acetyl | H | H | acetyl | acetyl | acetyl | H |
| 5017 | acetyl | acetyl | acetyl | acetyl | acetyl | H | H | acetyl | acetyl | acetyl | acetyl |
| 5018 | acetyl | acetyl | acetyl | acetyl | acetyl | H | acetyl | H | H | H | H |
| 5019 | acetyl | acetyl | acetyl | acetyl | acetyl | H | acetyl | H | H | H | acetyl |
| 5020 | acetyl | acetyl | acetyl | acetyl | acetyl | H | acetyl | H | H | acetyl | H |
| 5021 | acetyl | acetyl | acetyl | acetyl | acetyl | H | acetyl | H | H | acetyl | acetyl |
| 5022 | acetyl | acetyl | acetyl | acetyl | acetyl | H | acetyl | H | acetyl | H | H |
| 5023 | acetyl | acetyl | acetyl | acetyl | acetyl | H | acetyl | H | acetyl | H | acetyl |
| 5024 | acetyl | acetyl | acetyl | acetyl | acetyl | H | acetyl | H | acetyl | acetyl | H |
| 5025 | acetyl | acetyl | acetyl | acetyl | acetyl | H | acetyl | H | acetyl | acetyl | acetyl |
| 5026 | acetyl | acetyl | acetyl | acetyl | acetyl | H | acetyl | acetyl | H | H | H |
| 5027 | acetyl | acetyl | acetyl | acetyl | acetyl | H | acetyl | acetyl | H | H | acetyl |
| 5028 | acetyl | acetyl | acetyl | acetyl | acetyl | H | acetyl | acetyl | H | acetyl | H |
| 5029 | acetyl | acetyl | acetyl | acetyl | acetyl | H | acetyl | acetyl | H | acetyl | acetyl |
| 5030 | acetyl | acetyl | acetyl | acetyl | acetyl | H | acetyl | acetyl | acetyl | H | H |
| 5031 | acetyl | acetyl | acetyl | acetyl | acetyl | H | acetyl | acetyl | acetyl | H | acetyl |
| 5032 | acetyl | acetyl | acetyl | acetyl | acetyl | H | acetyl | acetyl | acetyl | acetyl | H |
| 5033 | acetyl | acetyl | acetyl | acetyl | acetyl | H | acetyl | acetyl | acetyl | acetyl | acetyl |
| 5034 | acetyl | acetyl | acetyl | acetyl | acetyl | acetyl | H | H | H | H | H |
| 5035 | acetyl | acetyl | acetyl | acetyl | acetyl | acetyl | H | H | H | H | acetyl |
| 5036 | acetyl | acetyl | acetyl | acetyl | acetyl | acetyl | H | H | H | acetyl | H |
| 5037 | acetyl | acetyl | acetyl | acetyl | acetyl | acetyl | H | H | H | acetyl | acetyl |
| 5038 | acetyl | acetyl | acetyl | acetyl | acetyl | acetyl | H | H | acetyl | H | H |
| 5039 | acetyl | acetyl | acetyl | acetyl | acetyl | acetyl | H | H | acetyl | H | acetyl |
| 5040 | acetyl | acetyl | acetyl | acetyl | acetyl | acetyl | H | H | acetyl | acetyl | H |
| 5041 | acetyl | acetyl | acetyl | acetyl | acetyl | acetyl | H | H | acetyl | acetyl | acetyl |
| 5042 | acetyl | acetyl | acetyl | acetyl | acetyl | acetyl | H | acetyl | H | H | H |
| 5043 | acetyl | acetyl | acetyl | acetyl | acetyl | acetyl | H | acetyl | H | H | acetyl |
| 5044 | acetyl | acetyl | acetyl | acetyl | acetyl | acetyl | H | acetyl | H | acetyl | H |
| 5045 | acetyl | acetyl | acetyl | acetyl | acetyl | acetyl | H | acetyl | H | acetyl | acetyl |
| 5046 | acetyl | acetyl | acetyl | acetyl | acetyl | acetyl | H | acetyl | acetyl | H | H |
| 5047 | acetyl | acetyl | acetyl | acetyl | acetyl | acetyl | H | acetyl | acetyl | H | acetyl |
| 5048 | acetyl | acetyl | acetyl | acetyl | acetyl | acetyl | H | acetyl | acetyl | acetyl | H |
| 5049 | acetyl | acetyl | acetyl | acetyl | acetyl | acetyl | H | acetyl | acetyl | acetyl | acetyl |
| 5050 | acetyl | acetyl | acetyl | acetyl | acetyl | acetyl | acetyl | H | H | H | H |
| 5051 | acetyl | acetyl | acetyl | acetyl | acetyl | acetyl | acetyl | H | H | H | acetyl |
| 5052 | acetyl | acetyl | acetyl | acetyl | acetyl | acetyl | acetyl | H | H | acetyl | H |
| 5053 | acetyl | acetyl | acetyl | acetyl | acetyl | acetyl | acetyl | H | H | acetyl | acetyl |
| 5054 | acetyl | acetyl | acetyl | acetyl | acetyl | acetyl | acetyl | H | acetyl | H | H |
| 5055 | acetyl | acetyl | acetyl | acetyl | acetyl | acetyl | acetyl | H | acetyl | H | acetyl |
| 5056 | acetyl | acetyl | acetyl | acetyl | acetyl | acetyl | acetyl | H | acetyl | acetyl | H |
| 5057 | acetyl | acetyl | acetyl | acetyl | acetyl | acetyl | acetyl | H | acetyl | acetyl | acetyl |
| 5058 | acetyl | acetyl | acetyl | acetyl | acetyl | acetyl | acetyl | acetyl | H | H | H |
| 5059 | acetyl | acetyl | acetyl | acetyl | acetyl | acetyl | acetyl | acetyl | H | H | acetyl |
| 5060 | acetyl | acetyl | acetyl | acetyl | acetyl | acetyl | acetyl | acetyl | H | acetyl | H |
| 5061 | acetyl | acetyl | acetyl | acetyl | acetyl | acetyl | acetyl | acetyl | H | acetyl | acetyl |
| 5062 | acetyl | acetyl | acetyl | acetyl | acetyl | acetyl | acetyl | acetyl | acetyl | H | H |
| 5063 | acetyl | acetyl | acetyl | acetyl | acetyl | acetyl | acetyl | acetyl | acetyl | H | acetyl |
| 5064 | acetyl | acetyl | acetyl | acetyl | acetyl | acetyl | acetyl | acetyl | acetyl | acetyl | H |

TABLE 9
(duodecasaccharides) (Y is either H or a carrier)

| | R2 | n1, R1 | n2, R1 | n3, R1 | n4, R1 | n5, R1 | n6, R1 | n7, R1 | n8, R1 | n9, R1 | n10; R1 | n11, R1 |
|---|---|---|---|---|---|---|---|---|---|---|---|---|
| 5065 | H | H | H | H | H | H | H | H | H | H | H | acetyl |
| 5066 | H | H | H | H | H | H | H | H | H | H | acetyl | H |
| 5067 | H | H | H | H | H | H | H | H | H | H | acetyl | acetyl |
| 5068 | H | H | H | H | H | H | H | H | H | acetyl | H | H |
| 5069 | H | H | H | H | H | H | H | H | H | acetyl | H | acetyl |
| 5070 | H | H | H | H | H | H | H | H | H | acetyl | acetyl | H |
| 5071 | H | H | H | H | H | H | H | H | H | acetyl | acetyl | acetyl |
| 5072 | H | H | H | H | H | H | H | H | acetyl | H | H | H |
| 5073 | H | H | H | H | H | H | H | H | acetyl | H | H | acetyl |
| 5074 | H | H | H | H | H | H | H | H | acetyl | H | acetyl | H |
| 5075 | H | H | H | H | H | H | H | H | acetyl | H | acetyl | acetyl |
| 5076 | H | H | H | H | H | H | H | H | acetyl | acetyl | H | H |
| 5077 | H | H | H | H | H | H | H | H | acetyl | acetyl | H | Acetyl |
| 5078 | H | H | H | H | H | H | H | H | acetyl | acetyl | acetyl | H |
| 5079 | H | H | H | H | H | H | H | H | acetyl | acetyl | acetyl | Acetyl |
| 5080 | H | H | H | H | H | H | H | acetyl | H | H | H | H |
| 5081 | H | H | H | H | H | H | H | acetyl | H | H | H | Acetyl |
| 5082 | H | H | H | H | H | H | H | acetyl | H | H | acetyl | H |
| 5083 | H | H | H | H | H | H | H | acetyl | H | H | acetyl | acetyl |
| 5084 | H | H | H | H | H | H | H | acetyl | H | acetyl | H | H |
| 5085 | H | H | H | H | H | H | H | acetyl | H | acetyl | H | acetyl |
| 5086 | H | H | H | H | H | H | H | acetyl | H | acetyl | acetyl | H |
| 5087 | H | H | H | H | H | H | H | acetyl | H | acetyl | acetyl | acetyl |
| 5088 | H | H | H | H | H | H | H | acetyl | acetyl | H | H | H |
| 5089 | H | H | H | H | H | H | H | acetyl | acetyl | H | H | acetyl |
| 5090 | H | H | H | H | H | H | H | acetyl | acetyl | H | acetyl | H |
| 5091 | H | H | H | H | H | H | H | acetyl | acetyl | H | acetyl | acetyl |
| 5092 | H | H | H | H | H | H | H | acetyl | acetyl | acetyl | H | H |
| 5093 | H | H | H | H | H | H | H | acetyl | acetyl | acetyl | H | acetyl |
| 5094 | H | H | H | H | H | H | H | acetyl | acetyl | acetyl | acetyl | H |
| 5095 | H | H | H | H | H | H | H | acetyl | acetyl | acetyl | acetyl | acetyl |
| 5096 | H | H | H | H | H | H | acetyl | H | H | H | H | H |
| 5097 | H | H | H | H | H | H | acetyl | H | H | H | H | acetyl |
| 5098 | H | H | H | H | H | H | acetyl | H | H | H | acetyl | H |
| 5099 | H | H | H | H | H | H | acetyl | H | H | H | acetyl | acetyl |
| 5100 | H | H | H | H | H | H | acetyl | H | H | acetyl | H | H |
| 5101 | H | H | H | H | H | H | acetyl | H | H | acetyl | H | acetyl |
| 5102 | H | H | H | H | H | H | acetyl | H | H | acetyl | acetyl | H |
| 5103 | H | H | H | H | H | H | acetyl | H | H | acetyl | acetyl | acetyl |
| 5104 | H | H | H | H | H | H | acetyl | H | acetyl | H | H | H |
| 5105 | H | H | H | H | H | H | acetyl | H | acetyl | H | H | acetyl |
| 5106 | H | H | H | H | H | H | acetyl | H | acetyl | H | acetyl | H |
| 5107 | H | H | H | H | H | H | acetyl | H | acetyl | H | acetyl | acetyl |
| 5108 | H | H | H | H | H | H | acetyl | H | acetyl | acetyl | H | H |
| 5109 | H | H | H | H | H | H | acetyl | H | acetyl | acetyl | H | acetyl |
| 5110 | H | H | H | H | H | H | acetyl | H | acetyl | acetyl | acetyl | H |
| 5111 | H | H | H | H | H | H | acetyl | H | acetyl | acetyl | acetyl | acetyl |
| 5112 | H | H | H | H | H | H | acetyl | acetyl | H | H | H | H |
| 5113 | H | H | H | H | H | H | acetyl | acetyl | H | H | H | acetyl |
| 5114 | H | H | H | H | H | H | acetyl | acetyl | H | H | acetyl | H |
| 5115 | H | H | H | H | H | H | acetyl | acetyl | H | H | acetyl | acetyl |
| 5116 | H | H | H | H | H | H | acetyl | acetyl | H | acetyl | H | H |
| 5117 | H | H | H | H | H | H | acetyl | acetyl | H | acetyl | H | acetyl |
| 5118 | H | H | H | H | H | H | acetyl | acetyl | H | acetyl | acetyl | H |
| 5119 | H | H | H | H | H | H | acetyl | acetyl | H | acetyl | acetyl | acetyl |
| 5120 | H | H | H | H | H | H | acetyl | acetyl | acetyl | H | H | H |
| 5121 | H | H | H | H | H | H | acetyl | acetyl | acetyl | H | H | acetyl |
| 5122 | H | H | H | H | H | H | acetyl | acetyl | acetyl | H | acetyl | H |
| 5123 | H | H | H | H | H | H | acetyl | acetyl | acetyl | H | acetyl | acetyl |
| 5124 | H | H | H | H | H | H | acetyl | acetyl | acetyl | acetyl | H | H |
| 5125 | H | H | H | H | H | H | acetyl | acetyl | acetyl | acetyl | H | acetyl |
| 5126 | H | H | H | H | H | H | acetyl | acetyl | acetyl | acetyl | acetyl | H |
| 5127 | H | H | H | H | H | H | acetyl | acetyl | acetyl | acetyl | acetyl | acetyl |
| 5128 | H | H | H | H | H | acetyl | H | H | H | H | H | H |
| 5129 | H | H | H | H | H | acetyl | H | H | H | H | H | acetyl |
| 5130 | H | H | H | H | H | acetyl | H | H | H | H | acetyl | H |
| 5131 | H | H | H | H | H | acetyl | H | H | H | H | acetyl | acetyl |
| 5132 | H | H | H | H | H | acetyl | H | H | H | acetyl | H | H |
| 5133 | H | H | H | H | H | acetyl | H | H | H | acetyl | H | acetyl |
| 5134 | H | H | H | H | H | acetyl | H | H | H | acetyl | acetyl | H |
| 5135 | H | H | H | H | H | acetyl | H | H | H | acetyl | acetyl | acetyl |
| 5136 | H | H | H | H | H | acetyl | H | H | acetyl | H | H | H |
| 5137 | H | H | H | H | H | acetyl | H | H | acetyl | H | H | acetyl |
| 5138 | H | H | H | H | H | acetyl | H | H | acetyl | H | acetyl | H |
| 5139 | H | H | H | H | H | acetyl | H | H | acetyl | H | acetyl | acetyl |
| 5140 | H | H | H | H | H | acetyl | H | H | acetyl | acetyl | H | H |
| 5141 | H | H | H | H | H | acetyl | H | H | acetyl | acetyl | H | acetyl |
| 5142 | H | H | H | H | H | acetyl | H | H | acetyl | acetyl | acetyl | H |
| 5143 | H | H | H | H | H | acetyl | H | H | acetyl | acetyl | acetyl | acetyl |
| 5144 | H | H | H | H | H | acetyl | H | acetyl | H | H | H | H |
| 5145 | H | H | H | H | H | acetyl | H | acetyl | H | H | H | acetyl |
| 5146 | H | H | H | H | H | acetyl | H | acetyl | H | H | acetyl | H |
| 5147 | H | H | H | H | H | acetyl | H | acetyl | H | H | acetyl | acetyl |
| 5148 | H | H | H | H | H | acetyl | H | acetyl | H | acetyl | H | H |
| 5149 | H | H | H | H | H | acetyl | H | acetyl | H | acetyl | H | acetyl |
| 5150 | H | H | H | H | H | acetyl | H | acetyl | H | acetyl | acetyl | H |
| 5151 | H | H | H | H | H | acetyl | H | acetyl | H | acetyl | acetyl | acetyl |
| 5152 | H | H | H | H | H | acetyl | H | acetyl | acetyl | H | H | H |
| 5153 | H | H | H | H | H | acetyl | H | acetyl | acetyl | H | H | acetyl |
| 5154 | H | H | H | H | H | acetyl | H | acetyl | acetyl | H | acetyl | H |
| 5155 | H | H | H | H | H | acetyl | H | acetyl | acetyl | H | acetyl | acetyl |
| 5156 | H | H | H | H | H | acetyl | H | acetyl | acetyl | acetyl | H | H |
| 5157 | H | H | H | H | H | acetyl | H | acetyl | acetyl | acetyl | H | acetyl |
| 5158 | H | H | H | H | H | acetyl | H | acetyl | acetyl | acetyl | acetyl | H |
| 5159 | H | H | H | H | H | acetyl | H | acetyl | acetyl | acetyl | acetyl | acetyl |
| 5160 | H | H | H | H | H | acetyl | acetyl | H | H | H | H | H |
| 5161 | H | H | H | H | H | acetyl | acetyl | H | H | H | H | acetyl |
| 5162 | H | H | H | H | H | acetyl | acetyl | H | H | H | acetyl | H |
| 5163 | H | H | H | H | H | acetyl | acetyl | H | H | H | acetyl | acetyl |
| 5164 | H | H | H | H | H | acetyl | acetyl | H | H | acetyl | H | H |
| 5165 | H | H | H | H | H | acetyl | acetyl | H | H | acetyl | H | acetyl |
| 5166 | H | H | H | H | H | acetyl | acetyl | H | H | acetyl | acetyl | H |
| 5167 | H | H | H | H | H | acetyl | acetyl | H | H | acetyl | acetyl | acetyl |
| 5168 | H | H | H | H | H | acetyl | acetyl | H | acetyl | H | H | H |
| 5169 | H | H | H | H | H | acetyl | acetyl | H | acetyl | H | H | acetyl |
| 5170 | H | H | H | H | H | acetyl | acetyl | H | acetyl | H | acetyl | H |
| 5171 | H | H | H | H | H | acetyl | acetyl | H | acetyl | H | acetyl | acetyl |
| 5172 | H | H | H | H | H | acetyl | acetyl | H | acetyl | acetyl | H | H |
| 5173 | H | H | H | H | H | acetyl | acetyl | H | acetyl | acetyl | H | acetyl |
| 5174 | H | H | H | H | H | acetyl | acetyl | H | acetyl | acetyl | acetyl | H |
| 5175 | H | H | H | H | H | acetyl | acetyl | H | acetyl | acetyl | acetyl | acetyl |
| 5176 | H | H | H | H | H | acetyl | acetyl | acetyl | H | H | H | H |
| 5177 | H | H | H | H | H | acetyl | acetyl | acetyl | H | H | H | acetyl |
| 5178 | H | H | H | H | H | acetyl | acetyl | acetyl | H | H | acetyl | H |
| 5179 | H | H | H | H | H | acetyl | acetyl | acetyl | H | H | acetyl | acetyl |
| 5180 | H | H | H | H | H | acetyl | acetyl | acetyl | H | acetyl | H | H |
| 5181 | H | H | H | H | H | acetyl | acetyl | acetyl | H | acetyl | H | acetyl |
| 5182 | H | H | H | H | H | acetyl | acetyl | acetyl | H | acetyl | acetyl | H |
| 5183 | H | H | H | H | H | acetyl | acetyl | acetyl | H | acetyl | acetyl | acetyl |
| 5184 | H | H | H | H | H | acetyl | acetyl | acetyl | acetyl | H | H | H |
| 5185 | H | H | H | H | H | acetyl | acetyl | acetyl | acetyl | H | H | acetyl |
| 5186 | H | H | H | H | H | acetyl | acetyl | acetyl | acetyl | H | acetyl | H |
| 5187 | H | H | H | H | H | acetyl | acetyl | acetyl | acetyl | H | acetyl | acetyl |
| 5188 | H | H | H | H | H | acetyl | acetyl | acetyl | acetyl | acetyl | H | H |
| 5189 | H | H | H | H | H | acetyl | acetyl | acetyl | acetyl | acetyl | H | acetyl |
| 5190 | H | H | H | H | H | acetyl | acetyl | acetyl | acetyl | acetyl | acetyl | H |
| 5191 | H | H | H | H | H | acetyl | acetyl | acetyl | acetyl | acetyl | acetyl | acetyl |
| 5192 | H | H | H | H | acetyl | H | H | H | H | H | H | H |
| 5193 | H | H | H | H | acetyl | H | H | H | H | H | H | acetyl |
| 5194 | H | H | H | H | acetyl | H | H | H | H | H | acetyl | H |
| 5195 | H | H | H | H | acetyl | H | H | H | H | H | acetyl | acetyl |
| 5196 | H | H | H | H | acetyl | H | H | H | H | acetyl | H | H |
| 5197 | H | H | H | H | acetyl | H | H | H | H | acetyl | H | acetyl |
| 5198 | H | H | H | H | acetyl | H | H | H | H | acetyl | acetyl | H |
| 5199 | H | H | H | H | acetyl | H | H | H | H | acetyl | acetyl | acetyl |
| 5200 | H | H | H | H | acetyl | H | H | H | acetyl | H | H | H |
| 5201 | H | H | H | H | acetyl | H | H | H | acetyl | H | H | acetyl |
| 5202 | H | H | H | H | acetyl | H | H | H | acetyl | H | acetyl | H |
| 5203 | H | H | H | H | acetyl | H | H | H | acetyl | H | acetyl | acetyl |
| 5204 | H | H | H | H | acetyl | H | H | H | acetyl | acetyl | H | H |
| 5205 | H | H | H | H | acetyl | H | H | H | acetyl | acetyl | H | acetyl |
| 5206 | H | H | H | H | acetyl | H | H | H | acetyl | acetyl | acetyl | H |
| 5207 | H | H | H | H | acetyl | H | H | H | acetyl | acetyl | acetyl | acetyl |
| 5208 | H | H | H | H | acetyl | H | H | acetyl | H | H | H | H |
| 5209 | H | H | H | H | acetyl | H | H | acetyl | H | H | H | acetyl |
| 5210 | H | H | H | H | acetyl | H | H | acetyl | H | H | acetyl | H |
| 5211 | H | H | H | H | acetyl | H | H | acetyl | H | H | acetyl | acetyl |
| 5212 | H | H | H | H | acetyl | H | H | acetyl | H | acetyl | H | H |
| 5213 | H | H | H | H | acetyl | H | H | acetyl | H | acetyl | H | acetyl |
| 5214 | H | H | H | H | acetyl | H | H | acetyl | H | acetyl | acetyl | H |
| 5215 | H | H | H | H | acetyl | H | H | acetyl | H | acetyl | acetyl | acetyl |
| 5216 | H | H | H | H | acetyl | H | H | acetyl | acetyl | H | H | H |
| 5217 | H | H | H | H | acetyl | H | H | acetyl | acetyl | H | H | acetyl |
| 5218 | H | H | H | H | acetyl | H | H | acetyl | acetyl | H | acetyl | H |
| 5219 | H | H | H | H | acetyl | H | H | acetyl | acetyl | H | acetyl | acetyl |
| 5220 | H | H | H | H | acetyl | H | H | acetyl | acetyl | acetyl | H | H |
| 5221 | H | H | H | H | acetyl | H | H | acetyl | acetyl | acetyl | H | acetyl |
| 5222 | H | H | H | H | acetyl | H | H | acetyl | acetyl | acetyl | acetyl | H |
| 5223 | H | H | H | H | acetyl | H | H | acetyl | acetyl | acetyl | acetyl | acetyl |
| 5224 | H | H | H | H | acetyl | H | acetyl | H | H | H | H | H |
| 5225 | H | H | H | H | acetyl | H | acetyl | H | H | H | H | acetyl |
| 5226 | H | H | H | H | acetyl | H | acetyl | H | H | H | acetyl | H |
| 5227 | H | H | H | H | acetyl | H | acetyl | H | H | H | acetyl | acetyl |
| 5228 | H | H | H | H | acetyl | H | acetyl | H | H | acetyl | H | H |
| 5229 | H | H | H | H | acetyl | H | acetyl | H | H | acetyl | H | acetyl |
| 5230 | H | H | H | H | acetyl | H | acetyl | H | H | acetyl | acetyl | H |
| 5231 | H | H | H | H | acetyl | H | acetyl | H | H | acetyl | acetyl | acetyl |
| 5232 | H | H | H | H | acetyl | H | acetyl | H | acetyl | H | H | H |
| 5233 | H | H | H | H | acetyl | H | acetyl | H | acetyl | H | H | acetyl |
| 5234 | H | H | H | H | acetyl | H | acetyl | H | acetyl | H | acetyl | H |
| 5235 | H | H | H | H | acetyl | H | acetyl | H | acetyl | H | acetyl | acetyl |
| 5236 | H | H | H | H | acetyl | H | acetyl | H | acetyl | acetyl | H | H |
| 5237 | H | H | H | H | acetyl | H | acetyl | H | acetyl | acetyl | H | acetyl |
| 5238 | H | H | H | H | acetyl | H | acetyl | H | acetyl | acetyl | acetyl | H |
| 5239 | H | H | H | H | acetyl | H | acetyl | H | acetyl | acetyl | acetyl | acetyl |
| 5240 | H | H | H | H | acetyl | H | acetyl | acetyl | H | H | H | H |
| 5241 | H | H | H | H | acetyl | H | acetyl | acetyl | H | H | H | acetyl |
| 5242 | H | H | H | H | acetyl | H | acetyl | acetyl | H | H | acetyl | H |
| 5243 | H | H | H | H | acetyl | H | acetyl | acetyl | H | H | acetyl | acetyl |
| 5244 | H | H | H | H | acetyl | H | acetyl | acetyl | H | acetyl | H | H |
| 5245 | H | H | H | H | acetyl | H | acetyl | acetyl | H | acetyl | H | acetyl |
| 5246 | H | H | H | H | acetyl | H | acetyl | acetyl | H | acetyl | acetyl | H |
| 5247 | H | H | H | H | acetyl | H | acetyl | acetyl | H | acetyl | acetyl | acetyl |
| 5248 | H | H | H | H | acetyl | H | acetyl | acetyl | acetyl | H | H | H |
| 5249 | H | H | H | H | acetyl | H | acetyl | acetyl | acetyl | H | H | acetyl |
| 5250 | H | H | H | H | acetyl | H | acetyl | acetyl | acetyl | H | acetyl | H |
| 5251 | H | H | H | H | acetyl | H | acetyl | acetyl | acetyl | H | acetyl | acetyl |
| 5252 | H | H | H | H | acetyl | H | acetyl | acetyl | acetyl | acetyl | H | H |
| 5253 | H | H | H | H | acetyl | H | acetyl | acetyl | acetyl | acetyl | H | acetyl |
| 5254 | H | H | H | H | acetyl | H | acetyl | acetyl | acetyl | acetyl | acetyl | H |
| 5255 | H | H | H | H | acetyl | H | acetyl | acetyl | acetyl | acetyl | acetyl | acetyl |
| 5256 | H | H | H | H | acetyl | acetyl | H | H | H | H | H | H |
| 5257 | H | H | H | H | acetyl | acetyl | H | H | H | H | H | acetyl |
| 5258 | H | H | H | H | acetyl | acetyl | H | H | H | H | acetyl | H |
| 5259 | H | H | H | H | acetyl | acetyl | H | H | H | H | acetyl | acetyl |
| 5260 | H | H | H | H | acetyl | acetyl | H | H | H | acetyl | H | H |
| 5261 | H | H | H | H | acetyl | acetyl | H | H | H | acetyl | H | acetyl |
| 5262 | H | H | H | H | acetyl | acetyl | H | H | H | acetyl | acetyl | H |
| 5263 | H | H | H | H | acetyl | acetyl | H | H | H | acetyl | acetyl | acetyl |
| 5264 | H | H | H | H | acetyl | acetyl | H | H | acetyl | H | H | H |
| 5265 | H | H | H | H | acetyl | acetyl | H | H | acetyl | H | H | acetyl |
| 5266 | H | H | H | H | acetyl | acetyl | H | H | acetyl | H | acetyl | H |
| 5267 | H | H | H | H | acetyl | acetyl | H | H | acetyl | H | acetyl | acetyl |
| 5268 | H | H | H | H | acetyl | acetyl | H | H | acetyl | acetyl | H | H |
| 5269 | H | H | H | H | acetyl | acetyl | H | H | acetyl | acetyl | H | acetyl |
| 5270 | H | H | H | H | acetyl | acetyl | H | H | acetyl | acetyl | acetyl | H |
| 5271 | H | H | H | H | acetyl | acetyl | H | H | acetyl | acetyl | acetyl | acetyl |
| 5272 | H | H | H | H | acetyl | acetyl | H | acetyl | H | H | H | H |
| 5273 | H | H | H | H | acetyl | acetyl | H | acetyl | H | H | H | acetyl |
| 5274 | H | H | H | H | acetyl | acetyl | H | acetyl | H | H | acetyl | H |
| 5275 | H | H | H | H | acetyl | acetyl | H | acetyl | H | H | acetyl | acetyl |
| 5276 | H | H | H | H | acetyl | acetyl | H | acetyl | H | acetyl | H | H |
| 5277 | H | H | H | H | acetyl | acetyl | H | acetyl | H | acetyl | H | acetyl |
| 5278 | H | H | H | H | acetyl | acetyl | H | acetyl | H | acetyl | acetyl | H |
| 5279 | H | H | H | H | acetyl | acetyl | H | acetyl | H | acetyl | acetyl | acetyl |
| 5280 | H | H | H | H | acetyl | acetyl | H | acetyl | acetyl | H | H | H |
| 5281 | H | H | H | H | acetyl | acetyl | H | acetyl | acetyl | H | H | acetyl |
| 5282 | H | H | H | H | acetyl | acetyl | H | acetyl | acetyl | H | acetyl | H |
| 5283 | H | H | H | H | acetyl | acetyl | H | acetyl | acetyl | H | acetyl | acetyl |
| 5284 | H | H | H | H | acetyl | acetyl | H | acetyl | acetyl | acetyl | H | H |
| 5285 | H | H | H | H | acetyl | acetyl | H | acetyl | acetyl | acetyl | H | acetyl |
| 5286 | H | H | H | H | acetyl | acetyl | H | acetyl | acetyl | acetyl | acetyl | H |
| 5287 | H | H | H | H | acetyl | acetyl | H | acetyl | acetyl | acetyl | acetyl | acetyl |
| 5288 | H | H | H | H | acetyl | acetyl | acetyl | H | H | H | H | H |
| 5289 | H | H | H | H | acetyl | acetyl | acetyl | H | H | H | H | acetyl |
| 5290 | H | H | H | H | acetyl | acetyl | acetyl | H | H | H | acetyl | H |
| 5291 | H | H | H | H | acetyl | acetyl | acetyl | H | H | H | acetyl | acetyl |
| 5292 | H | H | H | H | acetyl | acetyl | acetyl | H | H | acetyl | H | H |
| 5293 | H | H | H | H | acetyl | acetyl | acetyl | H | H | acetyl | H | acetyl |
| 5294 | H | H | H | H | acetyl | acetyl | acetyl | H | H | acetyl | acetyl | H |
| 5295 | H | H | H | H | acetyl | acetyl | acetyl | H | H | acetyl | acetyl | acetyl |
| 5296 | H | H | H | H | acetyl | acetyl | acetyl | H | acetyl | H | H | H |
| 5297 | H | H | H | H | acetyl | acetyl | acetyl | H | acetyl | H | H | acetyl |
| 5298 | H | H | H | H | acetyl | acetyl | acetyl | H | acetyl | H | acetyl | H |
| 5299 | H | H | H | H | acetyl | acetyl | acetyl | H | acetyl | H | acetyl | acetyl |
| 5300 | H | H | H | H | acetyl | acetyl | acetyl | H | acetyl | acetyl | H | H |
| 5301 | H | H | H | H | acetyl | acetyl | acetyl | H | acetyl | acetyl | H | acetyl |
| 5302 | H | H | H | H | acetyl | acetyl | acetyl | H | acetyl | acetyl | acetyl | H |
| 5303 | H | H | H | H | acetyl | acetyl | acetyl | H | acetyl | acetyl | acetyl | acetyl |
| 5304 | H | H | H | H | acetyl | acetyl | acetyl | acetyl | H | H | H | H |
| 5305 | H | H | H | H | acetyl | acetyl | acetyl | acetyl | H | H | H | acetyl |
| 5306 | H | H | H | H | acetyl | acetyl | acetyl | acetyl | H | H | acetyl | H |
| 5307 | H | H | H | H | acetyl | acetyl | acetyl | acetyl | H | H | acetyl | acetyl |
| 5308 | H | H | H | H | acetyl | acetyl | acetyl | acetyl | H | acetyl | H | H |
| 5309 | H | H | H | H | acetyl | acetyl | acetyl | acetyl | H | acetyl | H | acetyl |
| 5310 | H | H | H | H | acetyl | acetyl | acetyl | acetyl | H | acetyl | acetyl | H |
| 5311 | H | H | H | H | acetyl | acetyl | acetyl | acetyl | H | acetyl | acetyl | acetyl |
| 5312 | H | H | H | H | acetyl | acetyl | acetyl | acetyl | acetyl | H | H | H |
| 5313 | H | H | H | H | acetyl | acetyl | acetyl | acetyl | acetyl | H | H | acetyl |
| 5314 | H | H | H | H | acetyl | acetyl | acetyl | acetyl | acetyl | H | acetyl | H |
| 5315 | H | H | H | H | acetyl | acetyl | acetyl | acetyl | acetyl | H | acetyl | acetyl |
| 5316 | H | H | H | H | acetyl | acetyl | acetyl | acetyl | acetyl | acetyl | H | H |
| 5317 | H | H | H | H | acetyl | acetyl | acetyl | acetyl | acetyl | acetyl | H | acetyl |
| 5318 | H | H | H | H | acetyl | acetyl | acetyl | acetyl | acetyl | acetyl | acetyl | H |
| 5319 | H | H | H | H | acetyl | acetyl | acetyl | acetyl | acetyl | acetyl | acetyl | acetyl |
| 5320 | H | H | H | acetyl | H | H | H | H | H | H | H | H |
| 5321 | H | H | H | acetyl | H | H | H | H | H | H | H | acetyl |
| 5322 | H | H | H | acetyl | H | H | H | H | H | H | acetyl | H |
| 5323 | H | H | H | acetyl | H | H | H | H | H | H | acetyl | acetyl |
| 5324 | H | H | H | acetyl | H | H | H | H | H | acetyl | H | H |
| 5325 | H | H | H | acetyl | H | H | H | H | H | acetyl | H | acetyl |
| 5326 | H | H | H | acetyl | H | H | H | H | H | acetyl | acetyl | H |
| 5327 | H | H | H | acetyl | H | H | H | H | H | acetyl | acetyl | acetyl |
| 5328 | H | H | H | acetyl | H | H | H | H | acetyl | H | H | H |
| 5329 | H | H | H | acetyl | H | H | H | H | acetyl | H | H | acetyl |
| 5330 | H | H | H | acetyl | H | H | H | H | acetyl | H | acetyl | H |
| 5331 | H | H | H | acetyl | H | H | H | H | acetyl | H | acetyl | acetyl |
| 5332 | H | H | H | acetyl | H | H | H | H | acetyl | acetyl | H | H |
| 5333 | H | H | H | acetyl | H | H | H | H | acetyl | acetyl | H | acetyl |
| 5334 | H | H | H | acetyl | H | H | H | H | acetyl | acetyl | acetyl | H |
| 5335 | H | H | H | acetyl | H | H | H | H | acetyl | acetyl | acetyl | acetyl |
| 5336 | H | H | H | acetyl | H | H | H | acetyl | H | H | H | H |
| 5337 | H | H | H | acetyl | H | H | H | acetyl | H | H | H | acetyl |
| 5338 | H | H | H | acetyl | H | H | H | acetyl | H | H | acetyl | H |
| 5339 | H | H | H | acetyl | H | H | H | acetyl | H | H | acetyl | acetyl |
| 5340 | H | H | H | acetyl | H | H | H | acetyl | H | acetyl | H | H |
| 5341 | H | H | H | acetyl | H | H | H | acetyl | H | acetyl | H | acetyl |
| 5342 | H | H | H | acetyl | H | H | H | acetyl | H | acetyl | acetyl | H |
| 5343 | H | H | H | acetyl | H | H | H | acetyl | H | acetyl | acetyl | acetyl |
| 5344 | H | H | H | acetyl | H | H | H | acetyl | acetyl | H | H | H |
| 5345 | H | H | H | acetyl | H | H | H | acetyl | acetyl | H | H | acetyl |
| 5346 | H | H | H | acetyl | H | H | H | acetyl | acetyl | H | acetyl | H |
| 5347 | H | H | H | acetyl | H | H | H | acetyl | acetyl | H | acetyl | acetyl |
| 5348 | H | H | H | acetyl | H | H | H | acetyl | acetyl | acetyl | H | H |
| 5349 | H | H | H | acetyl | H | H | H | acetyl | acetyl | acetyl | H | acetyl |
| 5350 | H | H | H | acetyl | H | H | H | acetyl | acetyl | acetyl | acetyl | H |
| 5351 | H | H | H | acetyl | H | H | H | acetyl | acetyl | acetyl | acetyl | acetyl |
| 5352 | H | H | H | acetyl | H | H | acetyl | H | H | H | H | H |
| 5353 | H | H | H | acetyl | H | H | acetyl | H | H | H | H | acetyl |
| 5354 | H | H | H | acetyl | H | H | acetyl | H | H | H | acetyl | H |
| 5355 | H | H | H | acetyl | H | H | acetyl | H | H | H | acetyl | acetyl |
| 5356 | H | H | H | acetyl | H | H | acetyl | H | H | acetyl | H | H |
| 5357 | H | H | H | acetyl | H | H | acetyl | H | H | acetyl | H | acetyl |
| 5358 | H | H | H | acetyl | H | H | acetyl | H | H | acetyl | acetyl | H |
| 5359 | H | H | H | acetyl | H | H | acetyl | H | H | acetyl | acetyl | acetyl |
| 5360 | H | H | H | acetyl | H | H | acetyl | H | acetyl | H | H | H |
| 5361 | H | H | H | acetyl | H | H | acetyl | H | acetyl | H | H | acetyl |
| 5362 | H | H | H | acetyl | H | H | acetyl | H | acetyl | H | acetyl | H |
| 5363 | H | H | H | acetyl | H | H | acetyl | H | acetyl | H | acetyl | acetyl |
| 5364 | H | H | H | acetyl | H | H | acetyl | H | acetyl | acetyl | H | H |
| 5365 | H | H | H | acetyl | H | H | acetyl | H | acetyl | acetyl | H | acetyl |
| 5366 | H | H | H | acetyl | H | H | acetyl | H | acetyl | acetyl | acetyl | H |
| 5367 | H | H | H | acetyl | H | H | acetyl | H | acetyl | acetyl | acetyl | acetyl |
| 5368 | H | H | H | acetyl | H | H | acetyl | acetyl | H | H | H | H |
| 5369 | H | H | H | acetyl | H | H | acetyl | acetyl | H | H | H | acetyl |
| 5370 | H | H | H | acetyl | H | H | acetyl | acetyl | H | H | acetyl | H |
| 5371 | H | H | H | acetyl | H | H | acetyl | acetyl | H | H | acetyl | acetyl |
| 5372 | H | H | H | acetyl | H | H | acetyl | acetyl | H | acetyl | H | H |
| 5373 | H | H | H | acetyl | H | H | acetyl | acetyl | H | acetyl | H | acetyl |
| 5374 | H | H | H | acetyl | H | H | acetyl | acetyl | H | acetyl | acetyl | H |
| 5375 | H | H | H | acetyl | H | H | acetyl | acetyl | H | acetyl | acetyl | acetyl |
| 5376 | H | H | H | acetyl | H | H | acetyl | acetyl | acetyl | H | H | H |
| 5377 | H | H | H | acetyl | H | H | acetyl | acetyl | acetyl | H | H | acetyl |
| 5378 | H | H | H | acetyl | H | H | acetyl | acetyl | acetyl | H | acetyl | H |
| 5379 | H | H | H | acetyl | H | H | acetyl | acetyl | acetyl | H | acetyl | acetyl |
| 5380 | H | H | H | acetyl | H | H | acetyl | acetyl | acetyl | acetyl | H | H |
| 5381 | H | H | H | acetyl | H | H | acetyl | acetyl | acetyl | acetyl | H | acetyl |
| 5382 | H | H | H | acetyl | H | H | acetyl | acetyl | acetyl | acetyl | acetyl | H |
| 5383 | H | H | H | acetyl | H | H | acetyl | acetyl | acetyl | acetyl | acetyl | acetyl |
| 5384 | H | H | H | acetyl | H | acetyl | H | H | H | H | H | H |
| 5385 | H | H | H | acetyl | H | acetyl | H | H | H | H | H | acetyl |
| 5386 | H | H | H | acetyl | H | acetyl | H | H | H | H | acetyl | H |
| 5387 | H | H | H | acetyl | H | acetyl | H | H | H | H | acetyl | acetyl |
| 5388 | H | H | H | acetyl | H | acetyl | H | H | H | acetyl | H | H |
| 5389 | H | H | H | acetyl | H | acetyl | H | H | H | acetyl | H | acetyl |
| 5390 | H | H | H | acetyl | H | acetyl | H | H | H | acetyl | acetyl | H |
| 5391 | H | H | H | acetyl | H | acetyl | H | H | H | acetyl | acetyl | acetyl |
| 5392 | H | H | H | acetyl | H | acetyl | H | H | acetyl | H | H | H |
| 5393 | H | H | H | acetyl | H | acetyl | H | H | acetyl | H | H | acetyl |
| 5394 | H | H | H | acetyl | H | acetyl | H | H | acetyl | H | acetyl | H |
| 5395 | H | H | H | acetyl | H | acetyl | H | H | acetyl | H | acetyl | acetyl |
| 5396 | H | H | H | acetyl | H | acetyl | H | H | acetyl | acetyl | H | H |
| 5397 | H | H | H | acetyl | H | acetyl | H | H | acetyl | acetyl | H | acetyl |
| 5398 | H | H | H | acetyl | H | acetyl | H | H | acetyl | acetyl | acetyl | H |
| 5399 | H | H | H | acetyl | H | acetyl | H | H | acetyl | acetyl | acetyl | acetyl |
| 5400 | H | H | H | acetyl | H | acetyl | H | acetyl | H | H | H | H |
| 5401 | H | H | H | acetyl | H | acetyl | H | acetyl | H | H | H | acetyl |
| 5402 | H | H | H | acetyl | H | acetyl | H | acetyl | H | H | acetyl | H |
| 5403 | H | H | H | acetyl | H | acetyl | H | acetyl | H | H | acetyl | acetyl |
| 5404 | H | H | H | acetyl | H | acetyl | H | acetyl | H | acetyl | H | H |
| 5405 | H | H | H | acetyl | H | acetyl | H | acetyl | H | acetyl | H | acetyl |
| 5406 | H | H | H | acetyl | H | acetyl | H | acetyl | H | acetyl | acetyl | H |
| 5407 | H | H | H | acetyl | H | acetyl | H | acetyl | H | acetyl | acetyl | acetyl |
| 5408 | H | H | H | acetyl | H | acetyl | H | acetyl | acetyl | H | H | H |
| 5409 | H | H | H | acetyl | H | acetyl | H | acetyl | acetyl | H | H | acetyl |
| 5410 | H | H | H | acetyl | H | acetyl | H | acetyl | acetyl | H | acetyl | H |
| 5411 | H | H | H | acetyl | H | acetyl | H | acetyl | acetyl | H | acetyl | acetyl |
| 5412 | H | H | H | acetyl | H | acetyl | H | acetyl | acetyl | acetyl | H | H |
| 5413 | H | H | H | acetyl | H | acetyl | H | acetyl | acetyl | acetyl | H | acetyl |
| 5414 | H | H | H | acetyl | H | acetyl | H | acetyl | acetyl | acetyl | acetyl | H |
| 5415 | H | H | H | acetyl | H | acetyl | H | acetyl | acetyl | acetyl | acetyl | acetyl |
| 5416 | H | H | H | acetyl | H | acetyl | acetyl | H | H | H | H | H |
| 5417 | H | H | H | acetyl | H | acetyl | acetyl | H | H | H | H | acetyl |
| 5418 | H | H | H | acetyl | H | acetyl | acetyl | H | H | H | acetyl | H |
| 5419 | H | H | H | acetyl | H | acetyl | acetyl | H | H | H | acetyl | acetyl |
| 5420 | H | H | H | acetyl | H | acetyl | acetyl | H | H | acetyl | H | H |
| 5421 | H | H | H | acetyl | H | acetyl | acetyl | H | H | acetyl | H | acetyl |
| 5422 | H | H | H | acetyl | H | acetyl | acetyl | H | H | acetyl | acetyl | H |
| 5423 | H | H | H | acetyl | H | acetyl | acetyl | H | H | acetyl | acetyl | acetyl |
| 5424 | H | H | H | acetyl | H | acetyl | acetyl | H | acetyl | H | H | H |
| 5425 | H | H | H | acetyl | H | acetyl | acetyl | H | acetyl | H | H | acetyl |
| 5426 | H | H | H | acetyl | H | acetyl | acetyl | H | acetyl | H | acetyl | H |
| 5427 | H | H | H | acetyl | H | acetyl | acetyl | H | acetyl | H | acetyl | acetyl |
| 5428 | H | H | H | acetyl | H | acetyl | acetyl | H | acetyl | acetyl | H | H |
| 5429 | H | H | H | acetyl | H | acetyl | acetyl | H | acetyl | acetyl | H | acetyl |
| 5430 | H | H | H | acetyl | H | acetyl | acetyl | H | acetyl | acetyl | acetyl | H |
| 5431 | H | H | H | acetyl | H | acetyl | acetyl | H | acetyl | acetyl | acetyl | acetyl |
| 5432 | H | H | H | acetyl | H | acetyl | acetyl | acetyl | H | H | H | H |
| 5433 | H | H | H | acetyl | H | acetyl | acetyl | acetyl | H | H | H | acetyl |
| 5434 | H | H | H | acetyl | H | acetyl | acetyl | acetyl | H | H | acetyl | H |
| 5435 | H | H | H | acetyl | H | acetyl | acetyl | acetyl | H | H | acetyl | acetyl |
| 5436 | H | H | H | acetyl | H | acetyl | acetyl | acetyl | H | acetyl | H | H |
| 5437 | H | H | H | acetyl | H | acetyl | acetyl | acetyl | H | acetyl | H | acetyl |
| 5438 | H | H | H | acetyl | H | acetyl | acetyl | acetyl | H | acetyl | acetyl | H |
| 5439 | H | H | H | acetyl | H | acetyl | acetyl | acetyl | H | acetyl | acetyl | acetyl |
| 5440 | H | H | H | acetyl | H | acetyl | acetyl | acetyl | acetyl | H | H | H |
| 5441 | H | H | H | acetyl | H | acetyl | acetyl | acetyl | acetyl | H | H | acetyl |
| 5442 | H | H | H | acetyl | H | acetyl | acetyl | acetyl | acetyl | H | acetyl | H |
| 5443 | H | H | H | acetyl | H | acetyl | acetyl | acetyl | acetyl | H | acetyl | acetyl |
| 5444 | H | H | H | acetyl | H | acetyl | acetyl | acetyl | acetyl | acetyl | H | H |
| 5445 | H | H | H | acetyl | H | acetyl | acetyl | acetyl | acetyl | acetyl | H | acetyl |
| 5446 | H | H | H | acetyl | H | acetyl | acetyl | acetyl | acetyl | acetyl | acetyl | H |
| 5447 | H | H | H | acetyl | H | acetyl | acetyl | acetyl | acetyl | acetyl | acetyl | acetyl |
| 5448 | H | H | H | acetyl | acetyl | H | H | H | H | H | H | H |
| 5449 | H | H | H | acetyl | acetyl | H | H | H | H | H | H | acetyl |
| 5450 | H | H | H | acetyl | acetyl | H | H | H | H | H | acetyl | H |
| 5451 | H | H | H | acetyl | acetyl | H | H | H | H | H | acetyl | acetyl |
| 5452 | H | H | H | acetyl | acetyl | H | H | H | H | acetyl | H | H |
| 5453 | H | H | H | acetyl | acetyl | H | H | H | H | acetyl | H | acetyl |
| 5454 | H | H | H | acetyl | acetyl | H | H | H | H | acetyl | acetyl | H |
| 5455 | H | H | H | acetyl | acetyl | H | H | H | H | acetyl | acetyl | acetyl |
| 5456 | H | H | H | acetyl | acetyl | H | H | H | acetyl | H | H | H |
| 5457 | H | H | H | acetyl | acetyl | H | H | H | acetyl | H | H | acetyl |
| 5458 | H | H | H | acetyl | acetyl | H | H | H | acetyl | H | acetyl | H |
| 5459 | H | H | H | acetyl | acetyl | H | H | H | acetyl | H | acetyl | acetyl |
| 5460 | H | H | H | acetyl | acetyl | H | H | H | acetyl | acetyl | H | H |
| 5461 | H | H | H | acetyl | acetyl | H | H | H | acetyl | acetyl | H | acetyl |
| 5462 | H | H | H | acetyl | acetyl | H | H | H | acetyl | acetyl | acetyl | H |
| 5463 | H | H | H | acetyl | acetyl | H | H | H | acetyl | acetyl | acetyl | acetyl |
| 5464 | H | H | H | acetyl | acetyl | H | H | acetyl | H | H | H | H |
| 5465 | H | H | H | acetyl | acetyl | H | H | acetyl | H | H | H | acetyl |
| 5466 | H | H | H | acetyl | acetyl | H | H | acetyl | H | H | acetyl | H |
| 5467 | H | H | H | acetyl | acetyl | H | H | acetyl | H | H | acetyl | acetyl |
| 5468 | H | H | H | acetyl | acetyl | H | H | acetyl | H | acetyl | H | H |
| 5469 | H | H | H | acetyl | acetyl | H | H | acetyl | H | acetyl | H | acetyl |
| 5470 | H | H | H | acetyl | acetyl | H | H | acetyl | H | acetyl | acetyl | H |
| 5471 | H | H | H | acetyl | acetyl | H | H | acetyl | H | acetyl | acetyl | acetyl |
| 5472 | H | H | H | acetyl | acetyl | H | H | acetyl | acetyl | H | H | H |
| 5473 | H | H | H | acetyl | acetyl | H | H | acetyl | acetyl | H | H | acetyl |
| 5474 | H | H | H | acetyl | acetyl | H | H | acetyl | acetyl | H | acetyl | H |
| 5475 | H | H | H | acetyl | acetyl | H | H | acetyl | acetyl | H | acetyl | acetyl |
| 5476 | H | H | H | acetyl | acetyl | H | H | acetyl | acetyl | acetyl | H | H |
| 5477 | H | H | H | acetyl | acetyl | H | H | acetyl | acetyl | acetyl | H | acetyl |
| 5478 | H | H | H | acetyl | acetyl | H | H | acetyl | acetyl | acetyl | acetyl | H |
| 5479 | H | H | H | acetyl | acetyl | H | H | acetyl | acetyl | acetyl | acetyl | acetyl |
| 5480 | H | H | H | acetyl | acetyl | H | acetyl | H | H | H | H | H |
| 5481 | H | H | H | acetyl | acetyl | H | acetyl | H | H | H | H | acetyl |
| 5482 | H | H | H | acetyl | acetyl | H | acetyl | H | H | H | acetyl | H |
| 5483 | H | H | H | acetyl | acetyl | H | acetyl | H | H | H | acetyl | acetyl |
| 5484 | H | H | H | acetyl | acetyl | H | acetyl | H | H | acetyl | H | H |
| 5485 | H | H | H | acetyl | acetyl | H | acetyl | H | H | acetyl | H | acetyl |
| 5486 | H | H | H | acetyl | acetyl | H | acetyl | H | H | acetyl | acetyl | H |
| 5487 | H | H | H | acetyl | acetyl | H | acetyl | H | H | acetyl | acetyl | acetyl |
| 5488 | H | H | H | acetyl | acetyl | H | acetyl | H | acetyl | H | H | H |
| 5489 | H | H | H | acetyl | acetyl | H | acetyl | H | acetyl | H | H | acetyl |
| 5490 | H | H | H | acetyl | acetyl | H | acetyl | H | acetyl | H | acetyl | H |
| 5491 | H | H | H | acetyl | acetyl | H | acetyl | H | acetyl | H | acetyl | acetyl |
| 5492 | H | H | H | acetyl | acetyl | H | acetyl | H | acetyl | acetyl | H | H |
| 5493 | H | H | H | acetyl | acetyl | H | acetyl | H | acetyl | acetyl | H | acetyl |
| 5494 | H | H | H | acetyl | acetyl | H | acetyl | H | acetyl | acetyl | acetyl | H |
| 5495 | H | H | H | acetyl | acetyl | H | acetyl | H | acetyl | acetyl | acetyl | acetyl |
| 5496 | H | H | H | acetyl | acetyl | H | acetyl | acetyl | H | H | H | H |
| 5497 | H | H | H | acetyl | acetyl | H | acetyl | acetyl | H | H | H | acetyl |
| 5498 | H | H | H | acetyl | acetyl | H | acetyl | acetyl | H | H | acetyl | H |
| 5499 | H | H | H | acetyl | acetyl | H | acetyl | acetyl | H | H | acetyl | acetyl |
| 5500 | H | H | H | acetyl | acetyl | H | acetyl | acetyl | H | acetyl | H | H |
| 5501 | H | H | H | acetyl | acetyl | H | acetyl | acetyl | H | acetyl | H | acetyl |
| 5502 | H | H | H | acetyl | acetyl | H | acetyl | acetyl | H | acetyl | acetyl | H |
| 5503 | H | H | H | acetyl | acetyl | H | acetyl | acetyl | H | acetyl | acetyl | acetyl |
| 5504 | H | H | H | acetyl | acetyl | H | acetyl | acetyl | acetyl | H | H | H |
| 5505 | H | H | H | acetyl | acetyl | H | acetyl | acetyl | acetyl | H | H | acetyl |
| 5506 | H | H | H | acetyl | acetyl | H | acetyl | acetyl | acetyl | H | acetyl | H |
| 5507 | H | H | H | acetyl | acetyl | H | acetyl | acetyl | acetyl | H | acetyl | acetyl |
| 5508 | H | H | H | acetyl | acetyl | H | acetyl | acetyl | acetyl | acetyl | H | H |
| 5509 | H | H | H | acetyl | acetyl | H | acetyl | acetyl | acetyl | acetyl | H | acetyl |
| 5510 | H | H | H | acetyl | acetyl | H | acetyl | acetyl | acetyl | acetyl | acetyl | H |
| 5511 | H | H | H | acetyl | acetyl | H | acetyl | acetyl | acetyl | acetyl | acetyl | acetyl |
| 5512 | H | H | H | acetyl | acetyl | acetyl | H | H | H | H | H | H |
| 5513 | H | H | H | acetyl | acetyl | acetyl | H | H | H | H | H | acetyl |
| 5514 | H | H | H | acetyl | acetyl | acetyl | H | H | H | H | acetyl | H |
| 5515 | H | H | H | acetyl | acetyl | acetyl | H | H | H | H | acetyl | acetyl |
| 5516 | H | H | H | acetyl | acetyl | acetyl | H | H | H | acetyl | H | H |
| 5517 | H | H | H | acetyl | acetyl | acetyl | H | H | H | acetyl | H | acetyl |
| 5518 | H | H | H | acetyl | acetyl | acetyl | H | H | H | acetyl | acetyl | H |
| 5519 | H | H | H | acetyl | acetyl | acetyl | H | H | H | acetyl | acetyl | acetyl |
| 5520 | H | H | H | acetyl | acetyl | acetyl | H | H | acetyl | H | H | H |
| 5521 | H | H | H | acetyl | acetyl | acetyl | H | H | acetyl | H | H | acetyl |
| 5522 | H | H | H | acetyl | acetyl | acetyl | H | H | acetyl | H | acetyl | H |
| 5523 | H | H | H | acetyl | acetyl | acetyl | H | H | acetyl | H | acetyl | acetyl |
| 5524 | H | H | H | acetyl | acetyl | acetyl | H | H | acetyl | acetyl | H | H |
| 5525 | H | H | H | acetyl | acetyl | acetyl | H | H | acetyl | acetyl | H | acetyl |
| 5526 | H | H | H | acetyl | acetyl | acetyl | H | H | acetyl | acetyl | acetyl | H |
| 5527 | H | H | H | acetyl | acetyl | acetyl | H | H | acetyl | acetyl | acetyl | acetyl |
| 5528 | H | H | H | acetyl | acetyl | acetyl | H | acetyl | H | H | H | H |
| 5529 | H | H | H | acetyl | acetyl | acetyl | H | acetyl | H | H | H | acetyl |
| 5530 | H | H | H | acetyl | acetyl | acetyl | H | acetyl | H | H | acetyl | H |
| 5531 | H | H | H | acetyl | acetyl | acetyl | H | acetyl | H | H | acetyl | acetyl |
| 5532 | H | H | H | acetyl | acetyl | acetyl | H | acetyl | H | acetyl | H | H |
| 5533 | H | H | H | acetyl | acetyl | acetyl | H | acetyl | H | acetyl | H | acetyl |
| 5534 | H | H | H | acetyl | acetyl | acetyl | H | acetyl | H | acetyl | acetyl | H |
| 5535 | H | H | H | acetyl | acetyl | acetyl | H | acetyl | H | acetyl | acetyl | acetyl |
| 5536 | H | H | H | acetyl | acetyl | acetyl | H | acetyl | acetyl | H | H | H |
| 5537 | H | H | H | acetyl | acetyl | acetyl | H | acetyl | acetyl | H | H | acetyl |
| 5538 | H | H | H | acetyl | acetyl | acetyl | H | acetyl | acetyl | H | acetyl | H |
| 5539 | H | H | H | acetyl | acetyl | acetyl | H | acetyl | acetyl | H | acetyl | acetyl |
| 5540 | H | H | H | acetyl | acetyl | acetyl | H | acetyl | acetyl | acetyl | H | H |
| 5541 | H | H | H | acetyl | acetyl | acetyl | H | acetyl | acetyl | acetyl | H | acetyl |
| 5542 | H | H | H | acetyl | acetyl | acetyl | H | acetyl | acetyl | acetyl | acetyl | H |
| 5543 | H | H | H | acetyl | acetyl | acetyl | H | acetyl | acetyl | acetyl | acetyl | acetyl |
| 5544 | H | H | H | acetyl | acetyl | acetyl | acetyl | H | H | H | H | H |
| 5545 | H | H | H | acetyl | acetyl | acetyl | acetyl | H | H | H | H | acetyl |
| 5546 | H | H | H | acetyl | acetyl | acetyl | acetyl | H | H | H | acetyl | H |
| 5547 | H | H | H | acetyl | acetyl | acetyl | acetyl | H | H | H | acetyl | acetyl |
| 5548 | H | H | H | acetyl | acetyl | acetyl | acetyl | H | H | acetyl | H | H |
| 5549 | H | H | H | acetyl | acetyl | acetyl | acetyl | H | H | acetyl | H | acetyl |
| 5550 | H | H | H | acetyl | acetyl | acetyl | acetyl | H | H | acetyl | acetyl | H |
| 5551 | H | H | H | acetyl | acetyl | acetyl | acetyl | H | H | acetyl | acetyl | acetyl |
| 5552 | H | H | H | acetyl | acetyl | acetyl | acetyl | H | acetyl | H | H | H |
| 5553 | H | H | H | acetyl | acetyl | acetyl | acetyl | H | acetyl | H | H | acetyl |
| 5554 | H | H | H | acetyl | acetyl | acetyl | acetyl | H | acetyl | H | acetyl | H |
| 5555 | H | H | H | acetyl | acetyl | acetyl | acetyl | H | acetyl | H | acetyl | acetyl |
| 5556 | H | H | H | acetyl | acetyl | acetyl | acetyl | H | acetyl | acetyl | H | H |
| 5557 | H | H | H | acetyl | acetyl | acetyl | acetyl | H | acetyl | acetyl | H | acetyl |
| 5558 | H | H | H | acetyl | acetyl | acetyl | acetyl | H | acetyl | acetyl | acetyl | H |
| 5559 | H | H | H | acetyl | acetyl | acetyl | acetyl | H | acetyl | acetyl | acetyl | acetyl |
| 5560 | H | H | H | acetyl | acetyl | acetyl | acetyl | acetyl | H | H | H | H |
| 5561 | H | H | H | acetyl | acetyl | acetyl | acetyl | acetyl | H | H | H | acetyl |
| 5562 | H | H | H | acetyl | acetyl | acetyl | acetyl | acetyl | H | H | acetyl | H |
| 5563 | H | H | H | acetyl | acetyl | acetyl | acetyl | acetyl | H | H | acetyl | acetyl |
| 5564 | H | H | H | acetyl | acetyl | acetyl | acetyl | acetyl | H | acetyl | H | H |
| 5565 | H | H | H | acetyl | acetyl | acetyl | acetyl | acetyl | H | acetyl | H | acetyl |
| 5566 | H | H | H | acetyl | acetyl | acetyl | acetyl | acetyl | H | acetyl | acetyl | H |
| 5567 | H | H | H | acetyl | acetyl | acetyl | acetyl | acetyl | H | acetyl | acetyl | acetyl |
| 5568 | H | H | H | acetyl | acetyl | acetyl | acetyl | acetyl | acetyl | H | H | H |
| 5569 | H | H | H | acetyl | acetyl | acetyl | acetyl | acetyl | acetyl | H | H | acetyl |
| 5570 | H | H | H | acetyl | acetyl | acetyl | acetyl | acetyl | acetyl | H | acetyl | H |
| 5571 | H | H | H | acetyl | acetyl | acetyl | acetyl | acetyl | acetyl | H | acetyl | acetyl |
| 5572 | H | H | H | acetyl | acetyl | acetyl | acetyl | acetyl | acetyl | acetyl | H | H |
| 5573 | H | H | H | acetyl | acetyl | acetyl | acetyl | acetyl | acetyl | acetyl | H | acetyl |
| 5574 | H | H | H | acetyl | acetyl | acetyl | acetyl | acetyl | acetyl | acetyl | acetyl | H |
| 5575 | H | H | H | acetyl | acetyl | acetyl | acetyl | acetyl | acetyl | acetyl | acetyl | acetyl |
| 5576 | H | H | acetyl | H | H | H | H | H | H | H | H | H |
| 5577 | H | H | acetyl | H | H | H | H | H | H | H | H | acetyl |
| 5578 | H | H | acetyl | H | H | H | H | H | H | H | acetyl | H |
| 5579 | H | H | acetyl | H | H | H | H | H | H | H | acetyl | acetyl |
| 5580 | H | H | acetyl | H | H | H | H | H | H | acetyl | H | H |
| 5581 | H | H | acetyl | H | H | H | H | H | H | acetyl | H | acetyl |
| 5582 | H | H | acetyl | H | H | H | H | H | H | acetyl | acetyl | H |
| 5583 | H | H | acetyl | H | H | H | H | H | H | acetyl | acetyl | acetyl |
| 5584 | H | H | acetyl | H | H | H | H | H | acetyl | H | H | H |
| 5585 | H | H | acetyl | H | H | H | H | H | acetyl | H | H | acetyl |
| 5586 | H | H | acetyl | H | H | H | H | H | acetyl | H | acetyl | H |
| 5587 | H | H | acetyl | H | H | H | H | H | acetyl | H | acetyl | acetyl |
| 5588 | H | H | acetyl | H | H | H | H | H | acetyl | acetyl | H | H |
| 5589 | H | H | acetyl | H | H | H | H | H | acetyl | acetyl | H | acetyl |
| 5590 | H | H | acetyl | H | H | H | H | H | acetyl | acetyl | acetyl | H |
| 5591 | H | H | acetyl | H | H | H | H | H | acetyl | acetyl | acetyl | acetyl |
| 5592 | H | H | acetyl | H | H | H | H | acetyl | H | H | H | H |
| 5593 | H | H | acetyl | H | H | H | H | acetyl | H | H | H | acetyl |
| 5594 | H | H | acetyl | H | H | H | H | acetyl | H | H | acetyl | H |
| 5595 | H | H | acetyl | H | H | H | H | acetyl | H | H | acetyl | acetyl |
| 5596 | H | H | acetyl | H | H | H | H | acetyl | H | acetyl | H | H |
| 5597 | H | H | acetyl | H | H | H | H | acetyl | H | acetyl | H | acetyl |
| 5598 | H | H | acetyl | H | H | H | H | acetyl | H | acetyl | acetyl | H |
| 5599 | H | H | acetyl | H | H | H | H | acetyl | H | acetyl | acetyl | acetyl |
| 5600 | H | H | acetyl | H | H | H | H | acetyl | acetyl | H | H | H |
| 5601 | H | H | acetyl | H | H | H | H | acetyl | acetyl | H | H | acetyl |
| 5602 | H | H | acetyl | H | H | H | H | acetyl | acetyl | H | acetyl | H |
| 5603 | H | H | acetyl | H | H | H | H | acetyl | acetyl | H | acetyl | acetyl |
| 5604 | H | H | acetyl | H | H | H | H | acetyl | acetyl | acetyl | H | H |
| 5605 | H | H | acetyl | H | H | H | H | acetyl | acetyl | acetyl | H | acetyl |
| 5606 | H | H | acetyl | H | H | H | H | acetyl | acetyl | acetyl | acetyl | H |
| 5607 | H | H | acetyl | H | H | H | H | acetyl | acetyl | acetyl | acetyl | acetyl |
| 5608 | H | H | acetyl | H | H | H | acetyl | H | H | H | H | H |
| 5609 | H | H | acetyl | H | H | H | acetyl | H | H | H | H | acetyl |
| 5610 | H | H | acetyl | H | H | H | acetyl | H | H | H | acetyl | H |
| 5611 | H | H | acetyl | H | H | H | acetyl | H | H | H | acetyl | acetyl |
| 5612 | H | H | acetyl | H | H | H | acetyl | H | H | acetyl | H | H |
| 5613 | H | H | acetyl | H | H | H | acetyl | H | H | acetyl | H | acetyl |
| 5614 | H | H | acetyl | H | H | H | acetyl | H | H | acetyl | acetyl | H |
| 5615 | H | H | acetyl | H | H | H | acetyl | H | H | acetyl | acetyl | acetyl |
| 5616 | H | H | acetyl | H | H | H | acetyl | H | acetyl | H | H | H |
| 5617 | H | H | acetyl | H | H | H | acetyl | H | acetyl | H | H | acetyl |
| 5618 | H | H | acetyl | H | H | H | acetyl | H | acetyl | H | acetyl | H |
| 5619 | H | H | acetyl | H | H | H | acetyl | H | acetyl | H | acetyl | acetyl |
| 5620 | H | H | acetyl | H | H | H | acetyl | H | acetyl | acetyl | H | H |
| 5621 | H | H | acetyl | H | H | H | acetyl | H | acetyl | acetyl | H | acetyl |
| 5622 | H | H | acetyl | H | H | H | acetyl | H | acetyl | acetyl | acetyl | H |
| 5623 | H | H | acetyl | H | H | H | acetyl | H | acetyl | acetyl | acetyl | acetyl |
| 5624 | H | H | acetyl | H | H | H | acetyl | acetyl | H | H | H | H |
| 5625 | H | H | acetyl | H | H | H | acetyl | acetyl | H | H | H | acetyl |
| 5626 | H | H | acetyl | H | H | H | acetyl | acetyl | H | H | acetyl | H |
| 5627 | H | H | acetyl | H | H | H | acetyl | acetyl | H | H | acetyl | acetyl |
| 5628 | H | H | acetyl | H | H | H | acetyl | acetyl | H | acetyl | H | H |
| 5629 | H | H | acetyl | H | H | H | acetyl | acetyl | H | acetyl | H | acetyl |
| 5630 | H | H | acetyl | H | H | H | acetyl | acetyl | H | acetyl | acetyl | H |
| 5631 | H | H | acetyl | H | H | H | acetyl | acetyl | H | acetyl | acetyl | acetyl |
| 5632 | H | H | acetyl | H | H | H | acetyl | acetyl | acetyl | H | H | H |
| 5633 | H | H | acetyl | H | H | H | acetyl | acetyl | acetyl | H | H | acetyl |
| 5634 | H | H | acetyl | H | H | H | acetyl | acetyl | acetyl | H | acetyl | H |
| 5635 | H | H | acetyl | H | H | H | acetyl | acetyl | acetyl | H | acetyl | acetyl |
| 5636 | H | H | acetyl | H | H | H | acetyl | acetyl | acetyl | acetyl | H | H |
| 5637 | H | H | acetyl | H | H | H | acetyl | acetyl | acetyl | acetyl | H | acetyl |
| 5638 | H | H | acetyl | H | H | H | acetyl | acetyl | acetyl | acetyl | acetyl | H |
| 5639 | H | H | acetyl | H | H | H | acetyl | acetyl | acetyl | acetyl | acetyl | acetyl |
| 5640 | H | H | acetyl | H | H | acetyl | H | H | H | H | H | H |
| 5641 | H | H | acetyl | H | H | acetyl | H | H | H | H | H | acetyl |
| 5642 | H | H | acetyl | H | H | acetyl | H | H | H | H | acetyl | H |
| 5643 | H | H | acetyl | H | H | acetyl | H | H | H | H | acetyl | acetyl |
| 5644 | H | H | acetyl | H | H | acetyl | H | H | H | acetyl | H | H |
| 5645 | H | H | acetyl | H | H | acetyl | H | H | H | acetyl | H | acetyl |
| 5646 | H | H | acetyl | H | H | acetyl | H | H | H | acetyl | acetyl | H |
| 5647 | H | H | acetyl | H | H | acetyl | H | H | H | acetyl | acetyl | acetyl |
| 5648 | H | H | acetyl | H | H | acetyl | H | H | acetyl | H | H | H |
| 5649 | H | H | acetyl | H | H | acetyl | H | H | acetyl | H | H | acetyl |
| 5650 | H | H | acetyl | H | H | acetyl | H | H | acetyl | H | acetyl | H |
| 5651 | H | H | acetyl | H | H | acetyl | H | H | acetyl | H | acetyl | acetyl |
| 5652 | H | H | acetyl | H | H | acetyl | H | H | acetyl | acetyl | H | H |
| 5653 | H | H | acetyl | H | H | acetyl | H | H | acetyl | acetyl | H | acetyl |
| 5654 | H | H | acetyl | H | H | acetyl | H | H | acetyl | acetyl | acetyl | H |
| 5655 | H | H | acetyl | H | H | acetyl | H | H | acetyl | acetyl | acetyl | acetyl |
| 5656 | H | H | acetyl | H | H | acetyl | H | acetyl | H | H | H | H |
| 5657 | H | H | acetyl | H | H | acetyl | H | acetyl | H | H | H | acetyl |
| 5658 | H | H | acetyl | H | H | acetyl | H | acetyl | H | H | acetyl | H |
| 5659 | H | H | acetyl | H | H | acetyl | H | acetyl | H | H | acetyl | acetyl |
| 5660 | H | H | acetyl | H | H | acetyl | H | acetyl | H | acetyl | H | H |
| 5661 | H | H | acetyl | H | H | acetyl | H | acetyl | H | acetyl | H | acetyl |
| 5662 | H | H | acetyl | H | H | acetyl | H | acetyl | H | acetyl | acetyl | H |
| 5663 | H | H | acetyl | H | H | acetyl | H | acetyl | H | acetyl | acetyl | acetyl |
| 5664 | H | H | acetyl | H | H | acetyl | H | acetyl | acetyl | H | H | H |
| 5665 | H | H | acetyl | H | H | acetyl | H | acetyl | acetyl | H | H | acetyl |
| 5666 | H | H | acetyl | H | H | acetyl | H | acetyl | acetyl | H | acetyl | H |
| 5667 | H | H | acetyl | H | H | acetyl | H | acetyl | acetyl | H | acetyl | acetyl |
| 5668 | H | H | acetyl | H | H | acetyl | H | acetyl | acetyl | acetyl | H | H |
| 5669 | H | H | acetyl | H | H | acetyl | H | acetyl | acetyl | acetyl | H | acetyl |
| 5670 | H | H | acetyl | H | H | acetyl | H | acetyl | acetyl | acetyl | acetyl | H |
| 5671 | H | H | acetyl | H | H | acetyl | H | acetyl | acetyl | acetyl | acetyl | acetyl |
| 5672 | H | H | acetyl | H | H | acetyl | acetyl | H | H | H | H | H |
| 5673 | H | H | acetyl | H | H | acetyl | acetyl | H | H | H | H | acetyl |
| 5674 | H | H | acetyl | H | H | acetyl | acetyl | H | H | H | acetyl | H |
| 5675 | H | H | acetyl | H | H | acetyl | acetyl | H | H | H | acetyl | acetyl |
| 5676 | H | H | acetyl | H | H | acetyl | acetyl | H | H | acetyl | H | H |
| 5677 | H | H | acetyl | H | H | acetyl | acetyl | H | H | acetyl | H | acetyl |
| 5678 | H | H | acetyl | H | H | acetyl | acetyl | H | H | acetyl | acetyl | H |
| 5679 | H | H | acetyl | H | H | acetyl | acetyl | H | H | acetyl | acetyl | acetyl |
| 5680 | H | H | acetyl | H | H | acetyl | acetyl | H | acetyl | H | H | H |
| 5681 | H | H | acetyl | H | H | acetyl | acetyl | H | acetyl | H | H | acetyl |
| 5682 | H | H | acetyl | H | H | acetyl | acetyl | H | acetyl | H | acetyl | H |
| 5683 | H | H | acetyl | H | H | acetyl | acetyl | H | acetyl | H | acetyl | acetyl |
| 5684 | H | H | acetyl | H | H | acetyl | acetyl | H | acetyl | acetyl | H | H |
| 5685 | H | H | acetyl | H | H | acetyl | acetyl | H | acetyl | acetyl | H | acetyl |
| 5686 | H | H | acetyl | H | H | acetyl | acetyl | H | acetyl | acetyl | acetyl | H |
| 5687 | H | H | acetyl | H | H | acetyl | acetyl | H | acetyl | acetyl | acetyl | acetyl |
| 5688 | H | H | acetyl | H | H | acetyl | acetyl | acetyl | H | H | H | H |
| 5689 | H | H | acetyl | H | H | acetyl | acetyl | acetyl | H | H | H | acetyl |
| 5690 | H | H | acetyl | H | H | acetyl | acetyl | acetyl | H | H | acetyl | H |
| 5691 | H | H | acetyl | H | H | acetyl | acetyl | acetyl | H | H | acetyl | acetyl |
| 5692 | H | H | acetyl | H | H | acetyl | acetyl | acetyl | H | acetyl | H | H |
| 5693 | H | H | acetyl | H | H | acetyl | acetyl | acetyl | H | acetyl | H | acetyl |
| 5694 | H | H | acetyl | H | H | acetyl | acetyl | acetyl | H | acetyl | acetyl | H |
| 5695 | H | H | acetyl | H | H | acetyl | acetyl | acetyl | H | acetyl | acetyl | acetyl |
| 5696 | H | H | acetyl | H | H | acetyl | acetyl | acetyl | acetyl | H | H | H |
| 5697 | H | H | acetyl | H | H | acetyl | acetyl | acetyl | acetyl | H | H | acetyl |
| 5698 | H | H | acetyl | H | H | acetyl | acetyl | acetyl | acetyl | H | acetyl | H |
| 5699 | H | H | acetyl | H | H | acetyl | acetyl | acetyl | acetyl | H | acetyl | acetyl |
| 5700 | H | H | acetyl | H | H | acetyl | acetyl | acetyl | acetyl | acetyl | H | H |
| 5701 | H | H | acetyl | H | H | acetyl | acetyl | acetyl | acetyl | acetyl | H | acetyl |
| 5702 | H | H | acetyl | H | H | acetyl | acetyl | acetyl | acetyl | acetyl | acetyl | H |
| 5703 | H | H | acetyl | H | H | acetyl | acetyl | acetyl | acetyl | acetyl | acetyl | acetyl |
| 5704 | H | H | acetyl | H | acetyl | H | H | H | H | H | H | H |
| 5705 | H | H | acetyl | H | acetyl | H | H | H | H | H | H | acetyl |
| 5706 | H | H | acetyl | H | acetyl | H | H | H | H | H | acetyl | H |
| 5707 | H | H | acetyl | H | acetyl | H | H | H | H | H | acetyl | acetyl |
| 5708 | H | H | acetyl | H | acetyl | H | H | H | H | acetyl | H | H |
| 5709 | H | H | acetyl | H | acetyl | H | H | H | H | acetyl | H | acetyl |
| 5710 | H | H | acetyl | H | acetyl | H | H | H | H | acetyl | acetyl | H |
| 5711 | H | H | acetyl | H | acetyl | H | H | H | H | acetyl | acetyl | acetyl |
| 5712 | H | H | acetyl | H | acetyl | H | H | H | acetyl | H | H | H |
| 5713 | H | H | acetyl | H | acetyl | H | H | H | acetyl | H | H | acetyl |
| 5714 | H | H | acetyl | H | acetyl | H | H | H | acetyl | H | acetyl | H |
| 5715 | H | H | acetyl | H | acetyl | H | H | H | acetyl | H | acetyl | acetyl |
| 5716 | H | H | acetyl | H | acetyl | H | H | H | acetyl | acetyl | H | H |
| 5717 | H | H | acetyl | H | acetyl | H | H | H | acetyl | acetyl | H | acetyl |
| 5718 | H | H | acetyl | H | acetyl | H | H | H | acetyl | acetyl | acetyl | H |
| 5719 | H | H | acetyl | H | acetyl | H | H | H | acetyl | acetyl | acetyl | acetyl |
| 5720 | H | H | acetyl | H | acetyl | H | H | acetyl | H | H | H | H |
| 5721 | H | H | acetyl | H | acetyl | H | H | acetyl | H | H | H | acetyl |
| 5722 | H | H | acetyl | H | acetyl | H | H | acetyl | H | H | acetyl | H |
| 5723 | H | H | acetyl | H | acetyl | H | H | acetyl | H | H | acetyl | acetyl |
| 5724 | H | H | acetyl | H | acetyl | H | H | acetyl | H | acetyl | H | H |
| 5725 | H | H | acetyl | H | acetyl | H | H | acetyl | H | acetyl | H | acetyl |
| 5726 | H | H | acetyl | H | acetyl | H | H | acetyl | H | acetyl | acetyl | H |
| 5727 | H | H | acetyl | H | acetyl | H | H | acetyl | H | acetyl | acetyl | acetyl |
| 5728 | H | H | acetyl | H | acetyl | H | H | acetyl | acetyl | H | H | H |
| 5729 | H | H | acetyl | H | acetyl | H | H | acetyl | acetyl | H | H | acetyl |
| 5730 | H | H | acetyl | H | acetyl | H | H | acetyl | acetyl | H | acetyl | H |
| 5731 | H | H | acetyl | H | acetyl | H | H | acetyl | acetyl | H | acetyl | acetyl |
| 5732 | H | H | acetyl | H | acetyl | H | H | acetyl | acetyl | acetyl | H | H |
| 5733 | H | H | acetyl | H | acetyl | H | H | acetyl | acetyl | acetyl | H | acetyl |
| 5734 | H | H | acetyl | H | acetyl | H | H | acetyl | acetyl | acetyl | acetyl | H |
| 5735 | H | H | acetyl | H | acetyl | H | H | acetyl | acetyl | acetyl | acetyl | acetyl |
| 5736 | H | H | acetyl | H | acetyl | H | acetyl | H | H | H | H | H |
| 5737 | H | H | acetyl | H | acetyl | H | acetyl | H | H | H | H | acetyl |
| 5738 | H | H | acetyl | H | acetyl | H | acetyl | H | H | H | acetyl | H |
| 5739 | H | H | acetyl | H | acetyl | H | acetyl | H | H | H | acetyl | acetyl |
| 5740 | H | H | acetyl | H | acetyl | H | acetyl | H | H | acetyl | H | H |
| 5741 | H | H | acetyl | H | acetyl | H | acetyl | H | H | acetyl | H | acetyl |
| 5742 | H | H | acetyl | H | acetyl | H | acetyl | H | H | acetyl | acetyl | H |
| 5743 | H | H | acetyl | H | acetyl | H | acetyl | H | H | acetyl | acetyl | acetyl |
| 5744 | H | H | acetyl | H | acetyl | H | acetyl | H | acetyl | H | H | H |
| 5745 | H | H | acetyl | H | acetyl | H | acetyl | H | acetyl | H | H | acetyl |
| 5746 | H | H | acetyl | H | acetyl | H | acetyl | H | acetyl | H | acetyl | H |
| 5747 | H | H | acetyl | H | acetyl | H | acetyl | H | acetyl | H | acetyl | acetyl |
| 5748 | H | H | acetyl | H | acetyl | H | acetyl | H | acetyl | acetyl | H | H |
| 5749 | H | H | acetyl | H | acetyl | H | acetyl | H | acetyl | acetyl | H | acetyl |
| 5750 | H | H | acetyl | H | acetyl | H | acetyl | H | acetyl | acetyl | acetyl | H |
| 5751 | H | H | acetyl | H | acetyl | H | acetyl | H | acetyl | acetyl | acetyl | acetyl |
| 5752 | H | H | acetyl | H | acetyl | H | acetyl | acetyl | H | H | H | H |
| 5753 | H | H | acetyl | H | acetyl | H | acetyl | acetyl | H | H | H | acetyl |
| 5754 | H | H | acetyl | H | acetyl | H | acetyl | acetyl | H | H | acetyl | H |
| 5755 | H | H | acetyl | H | acetyl | H | acetyl | acetyl | H | H | acetyl | acetyl |
| 5756 | H | H | acetyl | H | acetyl | H | acetyl | acetyl | H | acetyl | H | H |
| 5757 | H | H | acetyl | H | acetyl | H | acetyl | acetyl | H | acetyl | H | acetyl |
| 5758 | H | H | acetyl | H | acetyl | H | acetyl | acetyl | H | acetyl | acetyl | H |
| 5759 | H | H | acetyl | H | acetyl | H | acetyl | acetyl | H | acetyl | acetyl | acetyl |
| 5760 | H | H | acetyl | H | acetyl | H | acetyl | acetyl | acetyl | H | H | H |
| 5761 | H | H | acetyl | H | acetyl | H | acetyl | acetyl | acetyl | H | H | acetyl |
| 5762 | H | H | acetyl | H | acetyl | H | acetyl | acetyl | acetyl | H | acetyl | H |
| 5763 | H | H | acetyl | H | acetyl | H | acetyl | acetyl | acetyl | H | acetyl | acetyl |
| 5764 | H | H | acetyl | H | acetyl | H | acetyl | acetyl | acetyl | acetyl | H | H |
| 5765 | H | H | acetyl | H | acetyl | H | acetyl | acetyl | acetyl | acetyl | H | acetyl |
| 5766 | H | H | acetyl | H | acetyl | H | acetyl | acetyl | acetyl | acetyl | acetyl | H |
| 5767 | H | H | acetyl | H | acetyl | H | acetyl | acetyl | acetyl | acetyl | acetyl | acetyl |
| 5768 | H | H | acetyl | H | acetyl | acetyl | H | H | H | H | H | H |
| 5769 | H | H | acetyl | H | acetyl | acetyl | H | H | H | H | H | acetyl |
| 5770 | H | H | acetyl | H | acetyl | acetyl | H | H | H | H | acetyl | H |
| 5771 | H | H | acetyl | H | acetyl | acetyl | H | H | H | H | acetyl | acetyl |
| 5772 | H | H | acetyl | H | acetyl | acetyl | H | H | H | acetyl | H | H |
| 5773 | H | H | acetyl | H | acetyl | acetyl | H | H | H | acetyl | H | acetyl |
| 5774 | H | H | acetyl | H | acetyl | acetyl | H | H | H | acetyl | acetyl | H |
| 5775 | H | H | acetyl | H | acetyl | acetyl | H | H | H | acetyl | acetyl | acetyl |
| 5776 | H | H | acetyl | H | acetyl | acetyl | H | H | acetyl | H | H | H |
| 5777 | H | H | acetyl | H | acetyl | acetyl | H | H | acetyl | H | H | acetyl |
| 5778 | H | H | acetyl | H | acetyl | acetyl | H | H | acetyl | H | acetyl | H |
| 5779 | H | H | acetyl | H | acetyl | acetyl | H | H | acetyl | H | acetyl | acetyl |
| 5780 | H | H | acetyl | H | acetyl | acetyl | H | H | acetyl | acetyl | H | H |
| 5781 | H | H | acetyl | H | acetyl | acetyl | H | H | acetyl | acetyl | H | acetyl |
| 5782 | H | H | acetyl | H | acetyl | acetyl | H | H | acetyl | acetyl | acetyl | H |
| 5783 | H | H | acetyl | H | acetyl | acetyl | H | H | acetyl | acetyl | acetyl | acetyl |
| 5784 | H | H | acetyl | H | acetyl | acetyl | H | acetyl | H | H | H | H |
| 5785 | H | H | acetyl | H | acetyl | acetyl | H | acetyl | H | H | H | acetyl |
| 5786 | H | H | acetyl | H | acetyl | acetyl | H | acetyl | H | H | acetyl | H |
| 5787 | H | H | acetyl | H | acetyl | acetyl | H | acetyl | H | H | acetyl | acetyl |
| 5788 | H | H | acetyl | H | acetyl | acetyl | H | acetyl | H | acetyl | H | H |
| 5789 | H | H | acetyl | H | acetyl | acetyl | H | acetyl | H | acetyl | H | acetyl |
| 5790 | H | H | acetyl | H | acetyl | acetyl | H | acetyl | H | acetyl | acetyl | H |
| 5791 | H | H | acetyl | H | acetyl | acetyl | H | acetyl | H | acetyl | acetyl | acetyl |
| 5792 | H | H | acetyl | H | acetyl | acetyl | H | acetyl | acetyl | H | H | H |
| 5793 | H | H | acetyl | H | acetyl | acetyl | H | acetyl | acetyl | H | H | acetyl |
| 5794 | H | H | acetyl | H | acetyl | acetyl | H | acetyl | acetyl | H | acetyl | H |
| 5795 | H | H | acetyl | H | acetyl | acetyl | H | acetyl | acetyl | H | acetyl | acetyl |
| 5796 | H | H | acetyl | H | acetyl | acetyl | H | acetyl | acetyl | acetyl | H | H |
| 5797 | H | H | acetyl | H | acetyl | acetyl | H | acetyl | acetyl | acetyl | H | acetyl |
| 5798 | H | H | acetyl | H | acetyl | acetyl | H | acetyl | acetyl | acetyl | acetyl | H |
| 5799 | H | H | acetyl | H | acetyl | acetyl | H | acetyl | acetyl | acetyl | acetyl | acetyl |
| 5800 | H | H | acetyl | H | acetyl | acetyl | acetyl | H | H | H | H | H |
| 5801 | H | H | acetyl | H | acetyl | acetyl | acetyl | H | H | H | H | acetyl |
| 5802 | H | H | acetyl | H | acetyl | acetyl | acetyl | H | H | H | acetyl | H |
| 5803 | H | H | acetyl | H | acetyl | acetyl | acetyl | H | H | H | acetyl | acetyl |
| 5804 | H | H | acetyl | H | acetyl | acetyl | acetyl | H | H | acetyl | H | H |
| 5805 | H | H | acetyl | H | acetyl | acetyl | acetyl | H | H | acetyl | H | acetyl |
| 5806 | H | H | acetyl | H | acetyl | acetyl | acetyl | H | H | acetyl | acetyl | H |
| 5807 | H | H | acetyl | H | acetyl | acetyl | acetyl | H | H | acetyl | acetyl | acetyl |
| 5808 | H | H | acetyl | H | acetyl | acetyl | acetyl | H | acetyl | H | H | H |
| 5809 | H | H | acetyl | H | acetyl | acetyl | acetyl | H | acetyl | H | H | acetyl |
| 5810 | H | H | acetyl | H | acetyl | acetyl | acetyl | H | acetyl | H | acetyl | H |
| 5811 | H | H | acetyl | H | acetyl | acetyl | acetyl | H | acetyl | H | acetyl | acetyl |
| 5812 | H | H | acetyl | H | acetyl | acetyl | acetyl | H | acetyl | acetyl | H | H |
| 5813 | H | H | acetyl | H | acetyl | acetyl | acetyl | H | acetyl | acetyl | H | acetyl |
| 5814 | H | H | acetyl | H | acetyl | acetyl | acetyl | H | acetyl | acetyl | acetyl | H |
| 5815 | H | H | acetyl | H | acetyl | acetyl | acetyl | H | acetyl | acetyl | acetyl | acetyl |
| 5816 | H | H | acetyl | H | acetyl | acetyl | acetyl | acetyl | H | H | H | H |
| 5817 | H | H | acetyl | H | acetyl | acetyl | acetyl | acetyl | H | H | H | acetyl |
| 5818 | H | H | acetyl | H | acetyl | acetyl | acetyl | acetyl | H | H | acetyl | H |
| 5819 | H | H | acetyl | H | acetyl | acetyl | acetyl | acetyl | H | H | acetyl | acetyl |
| 5820 | H | H | acetyl | H | acetyl | acetyl | acetyl | acetyl | H | acetyl | H | H |
| 5821 | H | H | acetyl | H | acetyl | acetyl | acetyl | acetyl | H | acetyl | H | acetyl |
| 5822 | H | H | acetyl | H | acetyl | acetyl | acetyl | acetyl | H | acetyl | acetyl | H |
| 5823 | H | H | acetyl | H | acetyl | acetyl | acetyl | acetyl | H | acetyl | acetyl | acetyl |
| 5824 | H | H | acetyl | H | acetyl | acetyl | acetyl | acetyl | acetyl | H | H | H |
| 5825 | H | H | acetyl | H | acetyl | acetyl | acetyl | acetyl | acetyl | H | H | acetyl |
| 5826 | H | H | acetyl | H | acetyl | acetyl | acetyl | acetyl | acetyl | H | acetyl | H |
| 5827 | H | H | acetyl | H | acetyl | acetyl | acetyl | acetyl | acetyl | H | acetyl | acetyl |
| 5828 | H | H | acetyl | H | acetyl | acetyl | acetyl | acetyl | acetyl | acetyl | H | H |
| 5829 | H | H | acetyl | H | acetyl | acetyl | acetyl | acetyl | acetyl | acetyl | H | acetyl |
| 5830 | H | H | acetyl | H | acetyl | acetyl | acetyl | acetyl | acetyl | acetyl | acetyl | H |
| 5831 | H | H | acetyl | H | acetyl | acetyl | acetyl | acetyl | acetyl | acetyl | acetyl | acetyl |
| 5832 | H | H | acetyl | acetyl | H | H | H | H | H | H | H | H |
| 5833 | H | H | acetyl | acetyl | H | H | H | H | H | H | H | acetyl |
| 5834 | H | H | acetyl | acetyl | H | H | H | H | H | H | acetyl | H |
| 5835 | H | H | acetyl | acetyl | H | H | H | H | H | H | acetyl | acetyl |
| 5836 | H | H | acetyl | acetyl | H | H | H | H | H | acetyl | H | H |
| 5837 | H | H | acetyl | acetyl | H | H | H | H | H | acetyl | H | acetyl |
| 5838 | H | H | acetyl | acetyl | H | H | H | H | H | acetyl | acetyl | H |
| 5839 | H | H | acetyl | acetyl | H | H | H | H | H | acetyl | acetyl | acetyl |
| 5840 | H | H | acetyl | acetyl | H | H | H | H | acetyl | H | H | H |
| 5841 | H | H | acetyl | acetyl | H | H | H | H | acetyl | H | H | acetyl |
| 5842 | H | H | acetyl | acetyl | H | H | H | H | acetyl | H | acetyl | H |
| 5843 | H | H | acetyl | acetyl | H | H | H | H | acetyl | H | acetyl | acetyl |
| 5844 | H | H | acetyl | acetyl | H | H | H | H | acetyl | acetyl | H | H |
| 5845 | H | H | acetyl | acetyl | H | H | H | H | acetyl | acetyl | H | acetyl |
| 5846 | H | H | acetyl | acetyl | H | H | H | H | acetyl | acetyl | acetyl | H |
| 5847 | H | H | acetyl | acetyl | H | H | H | H | acetyl | acetyl | acetyl | acetyl |
| 5848 | H | H | acetyl | acetyl | H | H | H | acetyl | H | H | H | H |
| 5849 | H | H | acetyl | acetyl | H | H | H | acetyl | H | H | H | acetyl |
| 5850 | H | H | acetyl | acetyl | H | H | H | acetyl | H | H | acetyl | H |
| 5851 | H | H | acetyl | acetyl | H | H | H | acetyl | H | H | acetyl | acetyl |
| 5852 | H | H | acetyl | acetyl | H | H | H | acetyl | H | acetyl | H | H |
| 5853 | H | H | acetyl | acetyl | H | H | H | acetyl | H | acetyl | H | acetyl |
| 5854 | H | H | acetyl | acetyl | H | H | H | acetyl | H | acetyl | acetyl | H |
| 5855 | H | H | acetyl | acetyl | H | H | H | acetyl | H | acetyl | acetyl | acetyl |
| 5856 | H | H | acetyl | acetyl | H | H | H | acetyl | acetyl | H | H | H |
| 5857 | H | H | acetyl | acetyl | H | H | H | acetyl | acetyl | H | H | acetyl |
| 5858 | H | H | acetyl | acetyl | H | H | H | acetyl | acetyl | H | acetyl | H |
| 5859 | H | H | acetyl | acetyl | H | H | H | acetyl | acetyl | H | acetyl | acetyl |
| 5860 | H | H | acetyl | acetyl | H | H | H | acetyl | acetyl | acetyl | H | H |
| 5861 | H | H | acetyl | acetyl | H | H | H | acetyl | acetyl | acetyl | H | acetyl |
| 5862 | H | H | acetyl | acetyl | H | H | H | acetyl | acetyl | acetyl | acetyl | H |
| 5863 | H | H | acetyl | acetyl | H | H | H | acetyl | acetyl | acetyl | acetyl | acetyl |
| 5864 | H | H | acetyl | acetyl | H | H | acetyl | H | H | H | H | H |
| 5865 | H | H | acetyl | acetyl | H | H | acetyl | H | H | H | H | acetyl |
| 5866 | H | H | acetyl | acetyl | H | H | acetyl | H | H | H | acetyl | H |
| 5867 | H | H | acetyl | acetyl | H | H | acetyl | H | H | H | acetyl | acetyl |
| 5868 | H | H | acetyl | acetyl | H | H | acetyl | H | H | acetyl | H | H |
| 5869 | H | H | acetyl | acetyl | H | H | acetyl | H | H | acetyl | H | acetyl |
| 5870 | H | H | acetyl | acetyl | H | H | acetyl | H | H | acetyl | acetyl | H |
| 5871 | H | H | acetyl | acetyl | H | H | acetyl | H | H | acetyl | acetyl | acetyl |
| 5872 | H | H | acetyl | acetyl | H | H | acetyl | H | acetyl | H | H | H |
| 5873 | H | H | acetyl | acetyl | H | H | acetyl | H | acetyl | H | H | acetyl |
| 5874 | H | H | acetyl | acetyl | H | H | acetyl | H | acetyl | H | acetyl | H |
| 5875 | H | H | acetyl | acetyl | H | H | acetyl | H | acetyl | H | acetyl | acetyl |
| 5876 | H | H | acetyl | acetyl | H | H | acetyl | H | acetyl | acetyl | H | H |
| 5877 | H | H | acetyl | acetyl | H | H | acetyl | H | acetyl | acetyl | H | acetyl |
| 5878 | H | H | acetyl | acetyl | H | H | acetyl | H | acetyl | acetyl | acetyl | H |
| 5879 | H | H | acetyl | acetyl | H | H | acetyl | H | acetyl | acetyl | acetyl | acetyl |
| 5880 | H | H | acetyl | acetyl | H | H | acetyl | acetyl | H | H | H | H |
| 5881 | H | H | acetyl | acetyl | H | H | acetyl | acetyl | H | H | H | acetyl |
| 5882 | H | H | acetyl | acetyl | H | H | acetyl | acetyl | H | H | acetyl | H |
| 5883 | H | H | acetyl | acetyl | H | H | acetyl | acetyl | H | H | acetyl | acetyl |
| 5884 | H | H | acetyl | acetyl | H | H | acetyl | acetyl | H | acetyl | H | H |
| 5885 | H | H | acetyl | acetyl | H | H | acetyl | acetyl | H | acetyl | H | acetyl |
| 5886 | H | H | acetyl | acetyl | H | H | acetyl | acetyl | H | acetyl | acetyl | H |
| 5887 | H | H | acetyl | acetyl | H | H | acetyl | acetyl | H | acetyl | acetyl | acetyl |
| 5888 | H | H | acetyl | acetyl | H | H | acetyl | acetyl | acetyl | H | H | H |
| 5889 | H | H | acetyl | acetyl | H | H | acetyl | acetyl | acetyl | H | H | acetyl |
| 5890 | H | H | acetyl | acetyl | H | H | acetyl | acetyl | acetyl | H | acetyl | H |
| 5891 | H | H | acetyl | acetyl | H | H | acetyl | acetyl | acetyl | H | acetyl | acetyl |
| 5892 | H | H | acetyl | acetyl | H | H | acetyl | acetyl | acetyl | acetyl | H | H |
| 5893 | H | H | acetyl | acetyl | H | H | acetyl | acetyl | acetyl | acetyl | H | acetyl |
| 5894 | H | H | acetyl | acetyl | H | H | acetyl | acetyl | acetyl | acetyl | acetyl | H |
| 5895 | H | H | acetyl | acetyl | H | H | acetyl | acetyl | acetyl | acetyl | acetyl | acetyl |
| 5896 | H | H | acetyl | acetyl | H | acetyl | H | H | H | H | H | H |
| 5897 | H | H | acetyl | acetyl | H | acetyl | H | H | H | H | H | acetyl |
| 5898 | H | H | acetyl | acetyl | H | acetyl | H | H | H | H | acetyl | H |
| 5899 | H | H | acetyl | acetyl | H | acetyl | H | H | H | H | acetyl | acetyl |
| 5900 | H | H | acetyl | acetyl | H | acetyl | H | H | H | acetyl | H | H |
| 5901 | H | H | acetyl | acetyl | H | acetyl | H | H | H | acetyl | H | acetyl |
| 5902 | H | H | acetyl | acetyl | H | acetyl | H | H | H | acetyl | acetyl | H |
| 5903 | H | H | acetyl | acetyl | H | acetyl | H | H | H | acetyl | acetyl | acetyl |
| 5904 | H | H | acetyl | acetyl | H | acetyl | H | H | acetyl | H | H | H |
| 5905 | H | H | acetyl | acetyl | H | acetyl | H | H | acetyl | H | H | acetyl |
| 5906 | H | H | acetyl | acetyl | H | acetyl | H | H | acetyl | H | acetyl | H |
| 5907 | H | H | acetyl | acetyl | H | acetyl | H | H | acetyl | H | acetyl | acetyl |
| 5908 | H | H | acetyl | acetyl | H | acetyl | H | H | acetyl | acetyl | H | H |
| 5909 | H | H | acetyl | acetyl | H | acetyl | H | H | acetyl | acetyl | H | acetyl |
| 5910 | H | H | acetyl | acetyl | H | acetyl | H | H | acetyl | acetyl | acetyl | H |
| 5911 | H | H | acetyl | acetyl | H | acetyl | H | H | acetyl | acetyl | acetyl | acetyl |
| 5912 | H | H | acetyl | acetyl | H | acetyl | H | acetyl | H | H | H | H |
| 5913 | H | H | acetyl | acetyl | H | acetyl | H | acetyl | H | H | H | acetyl |
| 5914 | H | H | acetyl | acetyl | H | acetyl | H | acetyl | H | H | acetyl | H |
| 5915 | H | H | acetyl | acetyl | H | acetyl | H | acetyl | H | H | acetyl | acetyl |
| 5916 | H | H | acetyl | acetyl | H | acetyl | H | acetyl | H | acetyl | H | H |
| 5917 | H | H | acetyl | acetyl | H | acetyl | H | acetyl | H | acetyl | H | acetyl |
| 5918 | H | H | acetyl | acetyl | H | acetyl | H | acetyl | H | acetyl | acetyl | H |
| 5919 | H | H | acetyl | acetyl | H | acetyl | H | acetyl | H | acetyl | acetyl | acetyl |
| 5920 | H | H | acetyl | acetyl | H | acetyl | H | acetyl | acetyl | H | H | H |
| 5921 | H | H | acetyl | acetyl | H | acetyl | H | acetyl | acetyl | H | H | acetyl |
| 5922 | H | H | acetyl | acetyl | H | acetyl | H | acetyl | acetyl | H | acetyl | H |
| 5923 | H | H | acetyl | acetyl | H | acetyl | H | acetyl | acetyl | H | acetyl | acetyl |
| 5924 | H | H | acetyl | acetyl | H | acetyl | H | acetyl | acetyl | acetyl | H | H |
| 5925 | H | H | acetyl | acetyl | H | acetyl | H | acetyl | acetyl | acetyl | H | acetyl |
| 5926 | H | H | acetyl | acetyl | H | acetyl | H | acetyl | acetyl | acetyl | acetyl | H |
| 5927 | H | H | acetyl | acetyl | H | acetyl | H | acetyl | acetyl | acetyl | acetyl | acetyl |
| 5928 | H | H | acetyl | acetyl | H | acetyl | acetyl | H | H | H | H | H |
| 5929 | H | H | acetyl | acetyl | H | acetyl | acetyl | H | H | H | H | acetyl |
| 5930 | H | H | acetyl | acetyl | H | acetyl | acetyl | H | H | H | acetyl | H |
| 5931 | H | H | acetyl | acetyl | H | acetyl | acetyl | H | H | H | acetyl | acetyl |
| 5932 | H | H | acetyl | acetyl | H | acetyl | acetyl | H | H | acetyl | H | H |
| 5933 | H | H | acetyl | acetyl | H | acetyl | acetyl | H | H | acetyl | H | acetyl |
| 5934 | H | H | acetyl | acetyl | H | acetyl | acetyl | H | H | acetyl | acetyl | H |
| 5935 | H | H | acetyl | acetyl | H | acetyl | acetyl | H | H | acetyl | acetyl | acetyl |
| 5936 | H | H | acetyl | acetyl | H | acetyl | acetyl | H | acetyl | H | H | H |
| 5937 | H | H | acetyl | acetyl | H | acetyl | acetyl | H | acetyl | H | H | acetyl |
| 5938 | H | H | acetyl | acetyl | H | acetyl | acetyl | H | acetyl | H | acetyl | H |
| 5939 | H | H | acetyl | acetyl | H | acetyl | acetyl | H | acetyl | H | acetyl | acetyl |
| 5940 | H | H | acetyl | acetyl | H | acetyl | acetyl | H | acetyl | acetyl | H | H |
| 5941 | H | H | acetyl | acetyl | H | acetyl | acetyl | H | acetyl | acetyl | H | acetyl |
| 5942 | H | H | acetyl | acetyl | H | acetyl | acetyl | H | acetyl | acetyl | acetyl | H |
| 5943 | H | H | acetyl | acetyl | H | acetyl | acetyl | H | acetyl | acetyl | acetyl | acetyl |
| 5944 | H | H | acetyl | acetyl | H | acetyl | acetyl | acetyl | H | H | H | H |
| 5945 | H | H | acetyl | acetyl | H | acetyl | acetyl | acetyl | H | H | H | acetyl |
| 5946 | H | H | acetyl | acetyl | H | acetyl | acetyl | acetyl | H | H | acetyl | H |
| 5947 | H | H | acetyl | acetyl | H | acetyl | acetyl | acetyl | H | H | acetyl | acetyl |
| 5948 | H | H | acetyl | acetyl | H | acetyl | acetyl | acetyl | H | acetyl | H | H |
| 5949 | H | H | acetyl | acetyl | H | acetyl | acetyl | acetyl | H | acetyl | H | acetyl |
| 5950 | H | H | acetyl | acetyl | H | acetyl | acetyl | acetyl | H | acetyl | acetyl | H |
| 5951 | H | H | acetyl | acetyl | H | acetyl | acetyl | acetyl | H | acetyl | acetyl | acetyl |
| 5952 | H | H | acetyl | acetyl | H | acetyl | acetyl | acetyl | acetyl | H | H | H |
| 5953 | H | H | acetyl | acetyl | H | acetyl | acetyl | acetyl | acetyl | H | H | acetyl |
| 5954 | H | H | acetyl | acetyl | H | acetyl | acetyl | acetyl | acetyl | H | acetyl | H |
| 5955 | H | H | acetyl | acetyl | H | acetyl | acetyl | acetyl | acetyl | H | acetyl | acetyl |
| 5956 | H | H | acetyl | acetyl | H | acetyl | acetyl | acetyl | acetyl | acetyl | H | H |
| 5957 | H | H | acetyl | acetyl | H | acetyl | acetyl | acetyl | acetyl | acetyl | H | acetyl |
| 5958 | H | H | acetyl | acetyl | H | acetyl | acetyl | acetyl | acetyl | acetyl | acetyl | H |
| 5959 | H | H | acetyl | acetyl | H | acetyl | acetyl | acetyl | acetyl | acetyl | acetyl | acetyl |
| 5960 | H | H | acetyl | acetyl | acetyl | H | H | H | H | H | H | H |
| 5961 | H | H | acetyl | acetyl | acetyl | H | H | H | H | H | H | acetyl |
| 5962 | H | H | acetyl | acetyl | acetyl | H | H | H | H | H | acetyl | H |
| 5963 | H | H | acetyl | acetyl | acetyl | H | H | H | H | H | acetyl | acetyl |
| 5964 | H | H | acetyl | acetyl | acetyl | H | H | H | H | acetyl | H | H |
| 5965 | H | H | acetyl | acetyl | acetyl | H | H | H | H | acetyl | H | acetyl |
| 5966 | H | H | acetyl | acetyl | acetyl | H | H | H | H | acetyl | acetyl | H |
| 5967 | H | H | acetyl | acetyl | acetyl | H | H | H | H | acetyl | acetyl | acetyl |
| 5968 | H | H | acetyl | acetyl | acetyl | H | H | H | acetyl | H | H | H |
| 5969 | H | H | acetyl | acetyl | acetyl | H | H | H | acetyl | H | H | acetyl |
| 5970 | H | H | acetyl | acetyl | acetyl | H | H | H | acetyl | H | acetyl | H |
| 5971 | H | H | acetyl | acetyl | acetyl | H | H | H | acetyl | H | acetyl | acetyl |
| 5972 | H | H | acetyl | acetyl | acetyl | H | H | H | acetyl | acetyl | H | H |
| 5973 | H | H | acetyl | acetyl | acetyl | H | H | H | acetyl | acetyl | H | acetyl |
| 5974 | H | H | acetyl | acetyl | acetyl | H | H | H | acetyl | acetyl | acetyl | H |
| 5975 | H | H | acetyl | acetyl | acetyl | H | H | H | acetyl | acetyl | acetyl | acetyl |
| 5976 | H | H | acetyl | acetyl | acetyl | H | H | acetyl | H | H | H | H |
| 5977 | H | H | acetyl | acetyl | acetyl | H | H | acetyl | H | H | H | acetyl |
| 5978 | H | H | acetyl | acetyl | acetyl | H | H | acetyl | H | H | acetyl | H |
| 5979 | H | H | acetyl | acetyl | acetyl | H | H | acetyl | H | H | acetyl | acetyl |
| 5980 | H | H | acetyl | acetyl | acetyl | H | H | acetyl | H | acetyl | H | H |
| 5981 | H | H | acetyl | acetyl | acetyl | H | H | acetyl | H | acetyl | H | acetyl |
| 5982 | H | H | acetyl | acetyl | acetyl | H | H | acetyl | H | acetyl | acetyl | H |
| 5983 | H | H | acetyl | acetyl | acetyl | H | H | acetyl | H | acetyl | acetyl | acetyl |
| 5984 | H | H | acetyl | acetyl | acetyl | H | H | acetyl | acetyl | H | H | H |
| 5985 | H | H | acetyl | acetyl | acetyl | H | H | acetyl | acetyl | H | H | acetyl |
| 5986 | H | H | acetyl | acetyl | acetyl | H | H | acetyl | acetyl | H | acetyl | H |
| 5987 | H | H | acetyl | acetyl | acetyl | H | H | acetyl | acetyl | H | acetyl | acetyl |
| 5988 | H | H | acetyl | acetyl | acetyl | H | H | acetyl | acetyl | acetyl | H | H |
| 5989 | H | H | acetyl | acetyl | acetyl | H | H | acetyl | acetyl | acetyl | H | acetyl |
| 5990 | H | H | acetyl | acetyl | acetyl | H | H | acetyl | acetyl | acetyl | acetyl | H |
| 5991 | H | H | acetyl | acetyl | acetyl | H | H | acetyl | acetyl | acetyl | acetyl | acetyl |
| 5992 | H | H | acetyl | acetyl | acetyl | H | acetyl | H | H | H | H | H |
| 5993 | H | H | acetyl | acetyl | acetyl | H | acetyl | H | H | H | H | acetyl |
| 5994 | H | H | acetyl | acetyl | acetyl | H | acetyl | H | H | H | acetyl | H |
| 5995 | H | H | acetyl | acetyl | acetyl | H | acetyl | H | H | H | acetyl | acetyl |
| 5996 | H | H | acetyl | acetyl | acetyl | H | acetyl | H | H | acetyl | H | H |
| 5997 | H | H | acetyl | acetyl | acetyl | H | acetyl | H | H | acetyl | H | acetyl |
| 5998 | H | H | acetyl | acetyl | acetyl | H | acetyl | H | H | acetyl | acetyl | H |
| 5999 | H | H | acetyl | acetyl | acetyl | H | acetyl | H | H | acetyl | acetyl | acetyl |
| 6000 | H | H | acetyl | acetyl | acetyl | H | acetyl | H | acetyl | H | H | H |
| 6001 | H | H | acetyl | acetyl | acetyl | H | acetyl | H | acetyl | H | H | acetyl |
| 6002 | H | H | acetyl | acetyl | acetyl | H | acetyl | H | acetyl | H | acetyl | H |
| 6003 | H | H | acetyl | acetyl | acetyl | H | acetyl | H | acetyl | H | acetyl | acetyl |
| 6004 | H | H | acetyl | acetyl | acetyl | H | acetyl | H | acetyl | acetyl | H | H |
| 6005 | H | H | acetyl | acetyl | acetyl | H | acetyl | H | acetyl | acetyl | H | acetyl |
| 6006 | H | H | acetyl | acetyl | acetyl | H | acetyl | H | acetyl | acetyl | acetyl | H |
| 6007 | H | H | acetyl | acetyl | acetyl | H | acetyl | H | acetyl | acetyl | acetyl | acetyl |
| 6008 | H | H | acetyl | acetyl | acetyl | H | acetyl | acetyl | H | H | H | H |
| 6009 | H | H | acetyl | acetyl | acetyl | H | acetyl | acetyl | H | H | H | acetyl |
| 6010 | H | H | acetyl | acetyl | acetyl | H | acetyl | acetyl | H | H | acetyl | H |
| 6011 | H | H | acetyl | acetyl | acetyl | H | acetyl | acetyl | H | H | acetyl | acetyl |
| 6012 | H | H | acetyl | acetyl | acetyl | H | acetyl | acetyl | H | acetyl | H | H |
| 6013 | H | H | acetyl | acetyl | acetyl | H | acetyl | acetyl | H | acetyl | H | acetyl |
| 6014 | H | H | acetyl | acetyl | acetyl | H | acetyl | acetyl | H | acetyl | acetyl | H |
| 6015 | H | H | acetyl | acetyl | acetyl | H | acetyl | acetyl | H | acetyl | acetyl | acetyl |
| 6016 | H | H | acetyl | acetyl | acetyl | H | acetyl | acetyl | acetyl | H | H | H |
| 6017 | H | H | acetyl | acetyl | acetyl | H | acetyl | acetyl | acetyl | H | H | acetyl |
| 6018 | H | H | acetyl | acetyl | acetyl | H | acetyl | acetyl | acetyl | H | acetyl | H |
| 6019 | H | H | acetyl | acetyl | acetyl | H | acetyl | acetyl | acetyl | H | acetyl | acetyl |
| 6020 | H | H | acetyl | acetyl | acetyl | H | acetyl | acetyl | acetyl | acetyl | H | H |
| 6021 | H | H | acetyl | acetyl | acetyl | H | acetyl | acetyl | acetyl | acetyl | H | acetyl |
| 6022 | H | H | acetyl | acetyl | acetyl | H | acetyl | acetyl | acetyl | acetyl | acetyl | H |
| 6023 | H | H | acetyl | acetyl | acetyl | H | acetyl | acetyl | acetyl | acetyl | acetyl | acetyl |
| 6024 | H | H | acetyl | acetyl | acetyl | acetyl | H | H | H | H | H | H |
| 6025 | H | H | acetyl | acetyl | acetyl | acetyl | H | H | H | H | H | acetyl |
| 6026 | H | H | acetyl | acetyl | acetyl | acetyl | H | H | H | H | acetyl | H |
| 6027 | H | H | acetyl | acetyl | acetyl | acetyl | H | H | H | H | acetyl | acetyl |
| 6028 | H | H | acetyl | acetyl | acetyl | acetyl | H | H | H | acetyl | H | H |
| 6029 | H | H | acetyl | acetyl | acetyl | acetyl | H | H | H | acetyl | H | acetyl |
| 6030 | H | H | acetyl | acetyl | acetyl | acetyl | H | H | H | acetyl | acetyl | H |
| 6031 | H | H | acetyl | acetyl | acetyl | acetyl | H | H | H | acetyl | acetyl | acetyl |
| 6032 | H | H | acetyl | acetyl | acetyl | acetyl | H | H | acetyl | H | H | H |
| 6033 | H | H | acetyl | acetyl | acetyl | acetyl | H | H | acetyl | H | H | acetyl |
| 6034 | H | H | acetyl | acetyl | acetyl | acetyl | H | H | acetyl | H | acetyl | H |
| 6035 | H | H | acetyl | acetyl | acetyl | acetyl | H | H | acetyl | H | acetyl | acetyl |
| 6036 | H | H | acetyl | acetyl | acetyl | acetyl | H | H | acetyl | acetyl | H | H |
| 6037 | H | H | acetyl | acetyl | acetyl | acetyl | H | H | acetyl | acetyl | H | acetyl |
| 6038 | H | H | acetyl | acetyl | acetyl | acetyl | H | H | acetyl | acetyl | acetyl | H |
| 6039 | H | H | acetyl | acetyl | acetyl | acetyl | H | H | acetyl | acetyl | acetyl | acetyl |
| 6040 | H | H | acetyl | acetyl | acetyl | acetyl | H | acetyl | H | H | H | H |
| 6041 | H | H | acetyl | acetyl | acetyl | acetyl | H | acetyl | H | H | H | acetyl |
| 6042 | H | H | acetyl | acetyl | acetyl | acetyl | H | acetyl | H | H | acetyl | H |
| 6043 | H | H | acetyl | acetyl | acetyl | acetyl | H | acetyl | H | H | acetyl | acetyl |
| 6044 | H | H | acetyl | acetyl | acetyl | acetyl | H | acetyl | H | acetyl | H | H |
| 6045 | H | H | acetyl | acetyl | acetyl | acetyl | H | acetyl | H | acetyl | H | acetyl |
| 6046 | H | H | acetyl | acetyl | acetyl | acetyl | H | acetyl | H | acetyl | acetyl | H |
| 6047 | H | H | acetyl | acetyl | acetyl | acetyl | H | acetyl | H | acetyl | acetyl | acetyl |
| 6048 | H | H | acetyl | acetyl | acetyl | acetyl | H | acetyl | acetyl | H | H | H |
| 6049 | H | H | acetyl | acetyl | acetyl | acetyl | H | acetyl | acetyl | H | H | acetyl |
| 6050 | H | H | acetyl | acetyl | acetyl | acetyl | H | acetyl | acetyl | H | acetyl | H |
| 6051 | H | H | acetyl | acetyl | acetyl | acetyl | H | acetyl | acetyl | H | acetyl | acetyl |
| 6052 | H | H | acetyl | acetyl | acetyl | acetyl | H | acetyl | acetyl | acetyl | H | H |
| 6053 | H | H | acetyl | acetyl | acetyl | acetyl | H | acetyl | acetyl | acetyl | H | acetyl |
| 6054 | H | H | acetyl | acetyl | acetyl | acetyl | H | acetyl | acetyl | acetyl | acetyl | H |
| 6055 | H | H | acetyl | acetyl | acetyl | acetyl | H | acetyl | acetyl | acetyl | acetyl | acetyl |
| 6056 | H | H | acetyl | acetyl | acetyl | acetyl | acetyl | H | H | H | H | H |
| 6057 | H | H | acetyl | acetyl | acetyl | acetyl | acetyl | H | H | H | H | acetyl |
| 6058 | H | H | acetyl | acetyl | acetyl | acetyl | acetyl | H | H | H | acetyl | H |
| 6059 | H | H | acetyl | acetyl | acetyl | acetyl | acetyl | H | H | H | acetyl | acetyl |
| 6060 | H | H | acetyl | acetyl | acetyl | acetyl | acetyl | H | H | acetyl | H | H |
| 6061 | H | H | acetyl | acetyl | acetyl | acetyl | acetyl | H | H | acetyl | H | acetyl |
| 6062 | H | H | acetyl | acetyl | acetyl | acetyl | acetyl | H | H | acetyl | acetyl | H |
| 6063 | H | H | acetyl | acetyl | acetyl | acetyl | acetyl | H | H | acetyl | acetyl | acetyl |
| 6064 | H | H | acetyl | acetyl | acetyl | acetyl | acetyl | H | acetyl | H | H | H |
| 6065 | H | H | acetyl | acetyl | acetyl | acetyl | acetyl | H | acetyl | H | H | acetyl |
| 6066 | H | H | acetyl | acetyl | acetyl | acetyl | acetyl | H | acetyl | H | acetyl | H |
| 6067 | H | H | acetyl | acetyl | acetyl | acetyl | acetyl | H | acetyl | H | acetyl | acetyl |
| 6068 | H | H | acetyl | acetyl | acetyl | acetyl | acetyl | H | acetyl | acetyl | H | H |
| 6069 | H | H | acetyl | acetyl | acetyl | acetyl | acetyl | H | acetyl | acetyl | H | acetyl |
| 6070 | H | H | acetyl | acetyl | acetyl | acetyl | acetyl | H | acetyl | acetyl | acetyl | H |
| 6071 | H | H | acetyl | acetyl | acetyl | acetyl | acetyl | H | acetyl | acetyl | acetyl | acetyl |
| 6072 | H | H | acetyl | acetyl | acetyl | acetyl | acetyl | acetyl | H | H | H | H |
| 6073 | H | H | acetyl | acetyl | acetyl | acetyl | acetyl | acetyl | H | H | H | acetyl |
| 6074 | H | H | acetyl | acetyl | acetyl | acetyl | acetyl | acetyl | H | H | acetyl | H |
| 6075 | H | H | acetyl | acetyl | acetyl | acetyl | acetyl | acetyl | H | H | acetyl | acetyl |
| 6076 | H | H | acetyl | acetyl | acetyl | acetyl | acetyl | acetyl | H | acetyl | H | H |
| 6077 | H | H | acetyl | acetyl | acetyl | acetyl | acetyl | acetyl | H | acetyl | H | acetyl |
| 6078 | H | H | acetyl | acetyl | acetyl | acetyl | acetyl | acetyl | H | acetyl | acetyl | H |
| 6079 | H | H | acetyl | acetyl | acetyl | acetyl | acetyl | acetyl | H | acetyl | acetyl | acetyl |
| 6080 | H | H | acetyl | acetyl | acetyl | acetyl | acetyl | acetyl | acetyl | H | H | H |
| 6081 | H | H | acetyl | acetyl | acetyl | acetyl | acetyl | acetyl | acetyl | H | H | acetyl |
| 6082 | H | H | acetyl | acetyl | acetyl | acetyl | acetyl | acetyl | acetyl | H | acetyl | H |
| 6083 | H | H | acetyl | acetyl | acetyl | acetyl | acetyl | acetyl | acetyl | H | acetyl | acetyl |
| 6084 | H | H | acetyl | acetyl | acetyl | acetyl | acetyl | acetyl | acetyl | acetyl | H | H |
| 6085 | H | H | acetyl | acetyl | acetyl | acetyl | acetyl | acetyl | acetyl | acetyl | H | acetyl |
| 6086 | H | H | acetyl | acetyl | acetyl | acetyl | acetyl | acetyl | acetyl | acetyl | acetyl | H |
| 6087 | H | H | acetyl | acetyl | acetyl | acetyl | acetyl | acetyl | acetyl | acetyl | acetyl | acetyl |
| 6088 | H | acetyl | H | H | H | H | H | H | H | H | H | H |
| 6089 | H | acetyl | H | H | H | H | H | H | H | H | H | acetyl |
| 6090 | H | acetyl | H | H | H | H | H | H | H | H | acetyl | H |
| 6091 | H | acetyl | H | H | H | H | H | H | H | H | acetyl | acetyl |
| 6092 | H | acetyl | H | H | H | H | H | H | H | acetyl | H | H |
| 6093 | H | acetyl | H | H | H | H | H | H | H | acetyl | H | acetyl |
| 6094 | H | acetyl | H | H | H | H | H | H | H | acetyl | acetyl | H |
| 6095 | H | acetyl | H | H | H | H | H | H | H | acetyl | acetyl | acetyl |
| 6096 | H | acetyl | H | H | H | H | H | H | acetyl | H | H | H |
| 6097 | H | acetyl | H | H | H | H | H | H | acetyl | H | H | acetyl |
| 6098 | H | acetyl | H | H | H | H | H | H | acetyl | H | acetyl | H |
| 6099 | H | acetyl | H | H | H | H | H | H | acetyl | H | acetyl | acetyl |
| 6100 | H | acetyl | H | H | H | H | H | H | acetyl | acetyl | H | H |
| 6101 | H | acetyl | H | H | H | H | H | H | acetyl | acetyl | H | acetyl |
| 6102 | H | acetyl | H | H | H | H | H | H | acetyl | acetyl | acetyl | H |
| 6103 | H | acetyl | H | H | H | H | H | H | acetyl | acetyl | acetyl | acetyl |
| 6104 | H | acetyl | H | H | H | H | H | acetyl | H | H | H | H |
| 6105 | H | acetyl | H | H | H | H | H | acetyl | H | H | H | acetyl |
| 6106 | H | acetyl | H | H | H | H | H | acetyl | H | H | acetyl | H |
| 6107 | H | acetyl | H | H | H | H | H | acetyl | H | H | acetyl | acetyl |
| 6108 | H | acetyl | H | H | H | H | H | acetyl | H | acetyl | H | H |
| 6109 | H | acetyl | H | H | H | H | H | acetyl | H | acetyl | H | acetyl |
| 6110 | H | acetyl | H | H | H | H | H | acetyl | H | acetyl | acetyl | H |
| 6111 | H | acetyl | H | H | H | H | H | acetyl | H | acetyl | acetyl | acetyl |
| 6112 | H | acetyl | H | H | H | H | H | acetyl | acetyl | H | H | H |
| 6113 | H | acetyl | H | H | H | H | H | acetyl | acetyl | H | H | acetyl |
| 6114 | H | acetyl | H | H | H | H | H | acetyl | acetyl | H | acetyl | H |
| 6115 | H | acetyl | H | H | H | H | H | acetyl | acetyl | H | acetyl | acetyl |
| 6116 | H | acetyl | H | H | H | H | H | acetyl | acetyl | acetyl | H | H |
| 6117 | H | acetyl | H | H | H | H | H | acetyl | acetyl | acetyl | H | acetyl |
| 6118 | H | acetyl | H | H | H | H | H | acetyl | acetyl | acetyl | acetyl | H |
| 6119 | H | acetyl | H | H | H | H | H | acetyl | acetyl | acetyl | acetyl | acetyl |
| 6120 | H | acetyl | H | H | H | H | acetyl | H | H | H | H | H |
| 6121 | H | acetyl | H | H | H | H | acetyl | H | H | H | H | acetyl |
| 6122 | H | acetyl | H | H | H | H | acetyl | H | H | H | acetyl | H |
| 6123 | H | acetyl | H | H | H | H | acetyl | H | H | H | acetyl | acetyl |
| 6124 | H | acetyl | H | H | H | H | acetyl | H | H | acetyl | H | H |
| 6125 | H | acetyl | H | H | H | H | acetyl | H | H | acetyl | H | acetyl |
| 6126 | H | acetyl | H | H | H | H | acetyl | H | H | acetyl | acetyl | H |
| 6127 | H | acetyl | H | H | H | H | acetyl | H | H | acetyl | acetyl | acetyl |
| 6128 | H | acetyl | H | H | H | H | acetyl | H | acetyl | H | H | H |
| 6129 | H | acetyl | H | H | H | H | acetyl | H | acetyl | H | H | acetyl |
| 6130 | H | acetyl | H | H | H | H | acetyl | H | acetyl | H | acetyl | H |
| 6131 | H | acetyl | H | H | H | H | acetyl | H | acetyl | H | acetyl | acetyl |
| 6132 | H | acetyl | H | H | H | H | acetyl | H | acetyl | acetyl | H | H |
| 6133 | H | acetyl | H | H | H | H | acetyl | H | acetyl | acetyl | H | acetyl |
| 6134 | H | acetyl | H | H | H | H | acetyl | H | acetyl | acetyl | acetyl | H |
| 6135 | H | acetyl | H | H | H | H | acetyl | H | acetyl | acetyl | acetyl | acetyl |
| 6136 | H | acetyl | H | H | H | H | acetyl | acetyl | H | H | H | H |
| 6137 | H | acetyl | H | H | H | H | acetyl | acetyl | H | H | H | acetyl |
| 6138 | H | acetyl | H | H | H | H | acetyl | acetyl | H | H | acetyl | H |
| 6139 | H | acetyl | H | H | H | H | acetyl | acetyl | H | H | acetyl | acetyl |
| 6140 | H | acetyl | H | H | H | H | acetyl | acetyl | H | acetyl | H | H |
| 6141 | H | acetyl | H | H | H | H | acetyl | acetyl | H | acetyl | H | acetyl |
| 6142 | H | acetyl | H | H | H | H | acetyl | acetyl | H | acetyl | acetyl | H |
| 6143 | H | acetyl | H | H | H | H | acetyl | acetyl | H | acetyl | acetyl | acetyl |
| 6144 | H | acetyl | H | H | H | H | acetyl | acetyl | acetyl | H | H | H |
| 6145 | H | acetyl | H | H | H | H | acetyl | acetyl | acetyl | H | H | acetyl |
| 6146 | H | acetyl | H | H | H | H | acetyl | acetyl | acetyl | H | acetyl | H |
| 6147 | H | acetyl | H | H | H | H | acetyl | acetyl | acetyl | H | acetyl | acetyl |
| 6148 | H | acetyl | H | H | H | H | acetyl | acetyl | acetyl | acetyl | H | H |
| 6149 | H | acetyl | H | H | H | H | acetyl | acetyl | acetyl | acetyl | H | acetyl |
| 6150 | H | acetyl | H | H | H | H | acetyl | acetyl | acetyl | acetyl | acetyl | H |
| 6151 | H | acetyl | H | H | H | H | acetyl | acetyl | acetyl | acetyl | acetyl | acetyl |
| 6152 | H | acetyl | H | H | H | acetyl | H | H | H | H | H | H |
| 6153 | H | acetyl | H | H | H | acetyl | H | H | H | H | H | acetyl |
| 6154 | H | acetyl | H | H | H | acetyl | H | H | H | H | acetyl | H |
| 6155 | H | acetyl | H | H | H | acetyl | H | H | H | H | acetyl | acetyl |
| 6156 | H | acetyl | H | H | H | acetyl | H | H | H | acetyl | H | H |
| 6157 | H | acetyl | H | H | H | acetyl | H | H | H | acetyl | H | acetyl |
| 6158 | H | acetyl | H | H | H | acetyl | H | H | H | acetyl | acetyl | H |
| 6159 | H | acetyl | H | H | H | acetyl | H | H | H | acetyl | acetyl | acetyl |
| 6160 | H | acetyl | H | H | H | acetyl | H | H | acetyl | H | H | H |
| 6161 | H | acetyl | H | H | H | acetyl | H | H | acetyl | H | H | acetyl |
| 6162 | H | acetyl | H | H | H | acetyl | H | H | acetyl | H | acetyl | H |
| 6163 | H | acetyl | H | H | H | acetyl | H | H | acetyl | H | acetyl | acetyl |
| 6164 | H | acetyl | H | H | H | acetyl | H | H | acetyl | acetyl | H | H |
| 6165 | H | acetyl | H | H | H | acetyl | H | H | acetyl | acetyl | H | acetyl |
| 6166 | H | acetyl | H | H | H | acetyl | H | H | acetyl | acetyl | acetyl | H |
| 6167 | H | acetyl | H | H | H | acetyl | H | H | acetyl | acetyl | acetyl | acetyl |
| 6168 | H | acetyl | H | H | H | acetyl | H | acetyl | H | H | H | H |
| 6169 | H | acetyl | H | H | H | acetyl | H | acetyl | H | H | H | acetyl |
| 6170 | H | acetyl | H | H | H | acetyl | H | acetyl | H | H | acetyl | H |
| 6171 | H | acetyl | H | H | H | acetyl | H | acetyl | H | H | acetyl | acetyl |
| 6172 | H | acetyl | H | H | H | acetyl | H | acetyl | H | acetyl | H | H |
| 6173 | H | acetyl | H | H | H | acetyl | H | acetyl | H | acetyl | H | acetyl |
| 6174 | H | acetyl | H | H | H | acetyl | H | acetyl | H | acetyl | acetyl | H |
| 6175 | H | acetyl | H | H | H | acetyl | H | acetyl | H | acetyl | acetyl | acetyl |
| 6176 | H | acetyl | H | H | H | acetyl | H | acetyl | acetyl | H | H | H |
| 6177 | H | acetyl | H | H | H | acetyl | H | acetyl | acetyl | H | H | acetyl |
| 6178 | H | acetyl | H | H | H | acetyl | H | acetyl | acetyl | H | acetyl | H |
| 6179 | H | acetyl | H | H | H | acetyl | H | acetyl | acetyl | H | acetyl | acetyl |
| 6180 | H | acetyl | H | H | H | acetyl | H | acetyl | acetyl | acetyl | H | H |
| 6181 | H | acetyl | H | H | H | acetyl | H | acetyl | acetyl | acetyl | H | acetyl |
| 6182 | H | acetyl | H | H | H | acetyl | H | acetyl | acetyl | acetyl | acetyl | H |
| 6183 | H | acetyl | H | H | H | acetyl | H | acetyl | acetyl | acetyl | acetyl | acetyl |
| 6184 | H | acetyl | H | H | H | acetyl | acetyl | H | H | H | H | H |
| 6185 | H | acetyl | H | H | H | acetyl | acetyl | H | H | H | H | acetyl |
| 6186 | H | acetyl | H | H | H | acetyl | acetyl | H | H | H | acetyl | H |
| 6187 | H | acetyl | H | H | H | acetyl | acetyl | H | H | H | acetyl | acetyl |
| 6188 | H | acetyl | H | H | H | acetyl | acetyl | H | H | acetyl | H | H |
| 6189 | H | acetyl | H | H | H | acetyl | acetyl | H | H | acetyl | H | acetyl |
| 6190 | H | acetyl | H | H | H | acetyl | acetyl | H | H | acetyl | acetyl | H |
| 6191 | H | acetyl | H | H | H | acetyl | acetyl | H | H | acetyl | acetyl | acetyl |
| 6192 | H | acetyl | H | H | H | acetyl | acetyl | H | acetyl | H | H | H |
| 6193 | H | acetyl | H | H | H | acetyl | acetyl | H | acetyl | H | H | acetyl |
| 6194 | H | acetyl | H | H | H | acetyl | acetyl | H | acetyl | H | acetyl | H |
| 6195 | H | acetyl | H | H | H | acetyl | acetyl | H | acetyl | H | acetyl | acetyl |
| 6196 | H | acetyl | H | H | H | acetyl | acetyl | H | acetyl | acetyl | H | H |
| 6197 | H | acetyl | H | H | H | acetyl | acetyl | H | acetyl | acetyl | H | acetyl |
| 6198 | H | acetyl | H | H | H | acetyl | acetyl | H | acetyl | acetyl | acetyl | H |
| 6199 | H | acetyl | H | H | H | acetyl | acetyl | H | acetyl | acetyl | acetyl | acetyl |
| 6200 | H | acetyl | H | H | H | acetyl | acetyl | acetyl | H | H | H | H |
| 6201 | H | acetyl | H | H | H | acetyl | acetyl | acetyl | H | H | H | acetyl |
| 6202 | H | acetyl | H | H | H | acetyl | acetyl | acetyl | H | H | acetyl | H |
| 6203 | H | acetyl | H | H | H | acetyl | acetyl | acetyl | H | H | acetyl | acetyl |
| 6204 | H | acetyl | H | H | H | acetyl | acetyl | acetyl | H | acetyl | H | H |
| 6205 | H | acetyl | H | H | H | acetyl | acetyl | acetyl | H | acetyl | H | acetyl |
| 6206 | H | acetyl | H | H | H | acetyl | acetyl | acetyl | H | acetyl | acetyl | H |
| 6207 | H | acetyl | H | H | H | acetyl | acetyl | acetyl | H | acetyl | acetyl | acetyl |
| 6208 | H | acetyl | H | H | H | acetyl | acetyl | acetyl | acetyl | H | H | H |
| 6209 | H | acetyl | H | H | H | acetyl | acetyl | acetyl | acetyl | H | H | acetyl |
| 6210 | H | acetyl | H | H | H | acetyl | acetyl | acetyl | acetyl | H | acetyl | H |
| 6211 | H | acetyl | H | H | H | acetyl | acetyl | acetyl | acetyl | H | acetyl | acetyl |
| 6212 | H | acetyl | H | H | H | acetyl | acetyl | acetyl | acetyl | acetyl | H | H |
| 6213 | H | acetyl | H | H | H | acetyl | acetyl | acetyl | acetyl | acetyl | H | acetyl |
| 6214 | H | acetyl | H | H | H | acetyl | acetyl | acetyl | acetyl | acetyl | acetyl | H |
| 6215 | H | acetyl | H | H | H | acetyl | acetyl | acetyl | acetyl | acetyl | acetyl | acetyl |
| 6216 | H | acetyl | H | H | acetyl | H | H | H | H | H | H | H |
| 6217 | H | acetyl | H | H | acetyl | H | H | H | H | H | H | acetyl |
| 6218 | H | acetyl | H | H | acetyl | H | H | H | H | H | acetyl | H |
| 6219 | H | acetyl | H | H | acetyl | H | H | H | H | H | acetyl | acetyl |
| 6220 | H | acetyl | H | H | acetyl | H | H | H | H | acetyl | H | H |
| 6221 | H | acetyl | H | H | acetyl | H | H | H | H | acetyl | H | acetyl |
| 6222 | H | acetyl | H | H | acetyl | H | H | H | H | acetyl | acetyl | H |
| 6223 | H | acetyl | H | H | acetyl | H | H | H | H | acetyl | acetyl | acetyl |
| 6224 | H | acetyl | H | H | acetyl | H | H | H | acetyl | H | H | H |
| 6225 | H | acetyl | H | H | acetyl | H | H | H | acetyl | H | H | acetyl |
| 6226 | H | acetyl | H | H | acetyl | H | H | H | acetyl | H | acetyl | H |
| 6227 | H | acetyl | H | H | acetyl | H | H | H | acetyl | H | acetyl | acetyl |
| 6228 | H | acetyl | H | H | acetyl | H | H | H | acetyl | acetyl | H | H |
| 6229 | H | acetyl | H | H | acetyl | H | H | H | acetyl | acetyl | H | acetyl |
| 6230 | H | acetyl | H | H | acetyl | H | H | H | acetyl | acetyl | acetyl | H |
| 6231 | H | acetyl | H | H | acetyl | H | H | H | acetyl | acetyl | acetyl | acetyl |
| 6232 | H | acetyl | H | H | acetyl | H | H | acetyl | H | H | H | H |
| 6233 | H | acetyl | H | H | acetyl | H | H | acetyl | H | H | H | acetyl |
| 6234 | H | acetyl | H | H | acetyl | H | H | acetyl | H | H | acetyl | H |
| 6235 | H | acetyl | H | H | acetyl | H | H | acetyl | H | H | acetyl | acetyl |
| 6236 | H | acetyl | H | H | acetyl | H | H | acetyl | H | acetyl | H | H |
| 6237 | H | acetyl | H | H | acetyl | H | H | acetyl | H | acetyl | H | acetyl |
| 6238 | H | acetyl | H | H | acetyl | H | H | acetyl | H | acetyl | acetyl | H |
| 6239 | H | acetyl | H | H | acetyl | H | H | acetyl | H | acetyl | acetyl | acetyl |
| 6240 | H | acetyl | H | H | acetyl | H | H | acetyl | acetyl | H | H | H |
| 6241 | H | acetyl | H | H | acetyl | H | H | acetyl | acetyl | H | H | acetyl |
| 6242 | H | acetyl | H | H | acetyl | H | H | acetyl | acetyl | H | acetyl | H |
| 6243 | H | acetyl | H | H | acetyl | H | H | acetyl | acetyl | H | acetyl | acetyl |
| 6244 | H | acetyl | H | H | acetyl | H | H | acetyl | acetyl | acetyl | H | H |
| 6245 | H | acetyl | H | H | acetyl | H | H | acetyl | acetyl | acetyl | H | acetyl |
| 6246 | H | acetyl | H | H | acetyl | H | H | acetyl | acetyl | acetyl | acetyl | H |
| 6247 | H | acetyl | H | H | acetyl | H | H | acetyl | acetyl | acetyl | acetyl | acetyl |
| 6248 | H | acetyl | H | H | acetyl | H | acetyl | H | H | H | H | H |
| 6249 | H | acetyl | H | H | acetyl | H | acetyl | H | H | H | H | acetyl |
| 6250 | H | acetyl | H | H | acetyl | H | acetyl | H | H | H | acetyl | H |
| 6251 | H | acetyl | H | H | acetyl | H | acetyl | H | H | H | acetyl | acetyl |
| 6252 | H | acetyl | H | H | acetyl | H | acetyl | H | H | acetyl | H | H |
| 6253 | H | acetyl | H | H | acetyl | H | acetyl | H | H | acetyl | H | acetyl |
| 6254 | H | acetyl | H | H | acetyl | H | acetyl | H | H | acetyl | acetyl | H |
| 6255 | H | acetyl | H | H | acetyl | H | acetyl | H | H | acetyl | acetyl | acetyl |
| 6256 | H | acetyl | H | H | acetyl | H | acetyl | H | acetyl | H | H | H |
| 6257 | H | acetyl | H | H | acetyl | H | acetyl | H | acetyl | H | H | acetyl |
| 6258 | H | acetyl | H | H | acetyl | H | acetyl | H | acetyl | H | acetyl | H |
| 6259 | H | acetyl | H | H | acetyl | H | acetyl | H | acetyl | H | acetyl | acetyl |
| 6260 | H | acetyl | H | H | acetyl | H | acetyl | H | acetyl | acetyl | H | H |
| 6261 | H | acetyl | H | H | acetyl | H | acetyl | H | acetyl | acetyl | H | acetyl |
| 6262 | H | acetyl | H | H | acetyl | H | acetyl | H | acetyl | acetyl | acetyl | H |
| 6263 | H | acetyl | H | H | acetyl | H | acetyl | H | acetyl | acetyl | acetyl | acetyl |
| 6264 | H | acetyl | H | H | acetyl | H | acetyl | acetyl | H | H | H | H |
| 6265 | H | acetyl | H | H | acetyl | H | acetyl | acetyl | H | H | H | acetyl |
| 6266 | H | acetyl | H | H | acetyl | H | acetyl | acetyl | H | H | acetyl | H |
| 6267 | H | acetyl | H | H | acetyl | H | acetyl | acetyl | H | H | acetyl | acetyl |
| 6268 | H | acetyl | H | H | acetyl | H | acetyl | acetyl | H | acetyl | H | H |
| 6269 | H | acetyl | H | H | acetyl | H | acetyl | acetyl | H | acetyl | H | acetyl |
| 6270 | H | acetyl | H | H | acetyl | H | acetyl | acetyl | H | acetyl | acetyl | H |
| 6271 | H | acetyl | H | H | acetyl | H | acetyl | acetyl | H | acetyl | acetyl | acetyl |
| 6272 | H | acetyl | H | H | acetyl | H | acetyl | acetyl | acetyl | H | H | H |
| 6273 | H | acetyl | H | H | acetyl | H | acetyl | acetyl | acetyl | H | H | acetyl |
| 6274 | H | acetyl | H | H | acetyl | H | acetyl | acetyl | acetyl | H | acetyl | H |
| 6275 | H | acetyl | H | H | acetyl | H | acetyl | acetyl | acetyl | H | acetyl | acetyl |
| 6276 | H | acetyl | H | H | acetyl | H | acetyl | acetyl | acetyl | acetyl | H | H |
| 6277 | H | acetyl | H | H | acetyl | H | acetyl | acetyl | acetyl | acetyl | H | acetyl |
| 6278 | H | acetyl | H | H | acetyl | H | acetyl | acetyl | acetyl | acetyl | acetyl | H |
| 6279 | H | acetyl | H | H | acetyl | H | acetyl | acetyl | acetyl | acetyl | acetyl | acetyl |
| 6280 | H | acetyl | H | H | acetyl | acetyl | H | H | H | H | H | H |
| 6281 | H | acetyl | H | H | acetyl | acetyl | H | H | H | H | H | acetyl |
| 6282 | H | acetyl | H | H | acetyl | acetyl | H | H | H | H | acetyl | H |
| 6283 | H | acetyl | H | H | acetyl | acetyl | H | H | H | H | acetyl | acetyl |
| 6284 | H | acetyl | H | H | acetyl | acetyl | H | H | H | acetyl | H | H |
| 6285 | H | acetyl | H | H | acetyl | acetyl | H | H | H | acetyl | H | acetyl |
| 6286 | H | acetyl | H | H | acetyl | acetyl | H | H | H | acetyl | acetyl | H |
| 6287 | H | acetyl | H | H | acetyl | acetyl | H | H | H | acetyl | acetyl | acetyl |
| 6288 | H | acetyl | H | H | acetyl | acetyl | H | H | acetyl | H | H | H |
| 6289 | H | acetyl | H | H | acetyl | acetyl | H | H | acetyl | H | H | acetyl |
| 6290 | H | acetyl | H | H | acetyl | acetyl | H | H | acetyl | H | acetyl | H |
| 6291 | H | acetyl | H | H | acetyl | acetyl | H | H | acetyl | H | acetyl | acetyl |
| 6292 | H | acetyl | H | H | acetyl | acetyl | H | H | acetyl | acetyl | H | H |
| 6293 | H | acetyl | H | H | acetyl | acetyl | H | H | acetyl | acetyl | H | acetyl |
| 6294 | H | acetyl | H | H | acetyl | acetyl | H | H | acetyl | acetyl | acetyl | H |
| 6295 | H | acetyl | H | H | acetyl | acetyl | H | H | acetyl | acetyl | acetyl | acetyl |
| 6296 | H | acetyl | H | H | acetyl | acetyl | H | acetyl | H | H | H | H |
| 6297 | H | acetyl | H | H | acetyl | acetyl | H | acetyl | H | H | H | acetyl |
| 6298 | H | acetyl | H | H | acetyl | acetyl | H | acetyl | H | H | acetyl | H |
| 6299 | H | acetyl | H | H | acetyl | acetyl | H | acetyl | H | H | acetyl | acetyl |
| 6300 | H | acetyl | H | H | acetyl | acetyl | H | acetyl | H | acetyl | H | H |
| 6301 | H | acetyl | H | H | acetyl | acetyl | H | acetyl | H | acetyl | H | acetyl |
| 6302 | H | acetyl | H | H | acetyl | acetyl | H | acetyl | H | acetyl | acetyl | H |
| 6303 | H | acetyl | H | H | acetyl | acetyl | H | acetyl | H | acetyl | acetyl | acetyl |
| 6304 | H | acetyl | H | H | acetyl | acetyl | H | acetyl | acetyl | H | H | H |
| 6305 | H | acetyl | H | H | acetyl | acetyl | H | acetyl | acetyl | H | H | acetyl |
| 6306 | H | acetyl | H | H | acetyl | acetyl | H | acetyl | acetyl | H | acetyl | H |
| 6307 | H | acetyl | H | H | acetyl | acetyl | H | acetyl | acetyl | H | acetyl | acetyl |
| 6308 | H | acetyl | H | H | acetyl | acetyl | H | acetyl | acetyl | acetyl | H | H |
| 6309 | H | acetyl | H | H | acetyl | acetyl | H | acetyl | acetyl | acetyl | H | acetyl |
| 6310 | H | acetyl | H | H | acetyl | acetyl | H | acetyl | acetyl | acetyl | acetyl | H |
| 6311 | H | acetyl | H | H | acetyl | acetyl | H | acetyl | acetyl | acetyl | acetyl | acetyl |
| 6312 | H | acetyl | H | H | acetyl | acetyl | acetyl | H | H | H | H | H |
| 6313 | H | acetyl | H | H | acetyl | acetyl | acetyl | H | H | H | H | acetyl |
| 6314 | H | acetyl | H | H | acetyl | acetyl | acetyl | H | H | H | acetyl | H |
| 6315 | H | acetyl | H | H | acetyl | acetyl | acetyl | H | H | H | acetyl | acetyl |
| 6316 | H | acetyl | H | H | acetyl | acetyl | acetyl | H | H | acetyl | H | H |
| 6317 | H | acetyl | H | H | acetyl | acetyl | acetyl | H | H | acetyl | H | acetyl |
| 6318 | H | acetyl | H | H | acetyl | acetyl | acetyl | H | H | acetyl | acetyl | H |
| 6319 | H | acetyl | H | H | acetyl | acetyl | acetyl | H | H | acetyl | acetyl | acetyl |
| 6320 | H | acetyl | H | H | acetyl | acetyl | acetyl | H | acetyl | H | H | H |
| 6321 | H | acetyl | H | H | acetyl | acetyl | acetyl | H | acetyl | H | H | acetyl |
| 6322 | H | acetyl | H | H | acetyl | acetyl | acetyl | H | acetyl | H | acetyl | H |
| 6323 | H | acetyl | H | H | acetyl | acetyl | acetyl | H | acetyl | H | acetyl | acetyl |
| 6324 | H | acetyl | H | H | acetyl | acetyl | acetyl | H | acetyl | acetyl | H | H |
| 6325 | H | acetyl | H | H | acetyl | acetyl | acetyl | H | acetyl | acetyl | H | acetyl |
| 6326 | H | acetyl | H | H | acetyl | acetyl | acetyl | H | acetyl | acetyl | acetyl | H |
| 6327 | H | acetyl | H | H | acetyl | acetyl | acetyl | H | acetyl | acetyl | acetyl | acetyl |
| 6328 | H | acetyl | H | H | acetyl | acetyl | acetyl | acetyl | H | H | H | H |
| 6329 | H | acetyl | H | H | acetyl | acetyl | acetyl | acetyl | H | H | H | acetyl |
| 6330 | H | acetyl | H | H | acetyl | acetyl | acetyl | acetyl | H | H | acetyl | H |
| 6331 | H | acetyl | H | H | acetyl | acetyl | acetyl | acetyl | H | H | acetyl | acetyl |
| 6332 | H | acetyl | H | H | acetyl | acetyl | acetyl | acetyl | H | acetyl | H | H |
| 6333 | H | acetyl | H | H | acetyl | acetyl | acetyl | acetyl | H | acetyl | H | acetyl |
| 6334 | H | acetyl | H | H | acetyl | acetyl | acetyl | acetyl | H | acetyl | acetyl | H |
| 6335 | H | acetyl | H | H | acetyl | acetyl | acetyl | acetyl | H | acetyl | acetyl | acetyl |
| 6336 | H | acetyl | H | H | acetyl | acetyl | acetyl | acetyl | acetyl | H | H | H |
| 6337 | H | acetyl | H | H | acetyl | acetyl | acetyl | acetyl | acetyl | H | H | acetyl |
| 6338 | H | acetyl | H | H | acetyl | acetyl | acetyl | acetyl | acetyl | H | acetyl | H |
| 6339 | H | acetyl | H | H | acetyl | acetyl | acetyl | acetyl | acetyl | H | acetyl | acetyl |
| 6340 | H | acetyl | H | H | acetyl | acetyl | acetyl | acetyl | acetyl | acetyl | H | H |
| 6341 | H | acetyl | H | H | acetyl | acetyl | acetyl | acetyl | acetyl | acetyl | H | acetyl |
| 6342 | H | acetyl | H | H | acetyl | acetyl | acetyl | acetyl | acetyl | acetyl | acetyl | H |
| 6343 | H | acetyl | H | H | acetyl | acetyl | acetyl | acetyl | acetyl | acetyl | acetyl | acetyl |
| 6344 | H | acetyl | H | acetyl | H | H | H | H | H | H | H | H |
| 6345 | H | acetyl | H | acetyl | H | H | H | H | H | H | H | acetyl |
| 6346 | H | acetyl | H | acetyl | H | H | H | H | H | H | acetyl | H |
| 6347 | H | acetyl | H | acetyl | H | H | H | H | H | H | acetyl | acetyl |
| 6348 | H | acetyl | H | acetyl | H | H | H | H | H | acetyl | H | H |
| 6349 | H | acetyl | H | acetyl | H | H | H | H | H | acetyl | H | acetyl |
| 6350 | H | acetyl | H | acetyl | H | H | H | H | H | acetyl | acetyl | H |
| 6351 | H | acetyl | H | acetyl | H | H | H | H | H | acetyl | acetyl | acetyl |
| 6352 | H | acetyl | H | acetyl | H | H | H | H | acetyl | H | H | H |
| 6353 | H | acetyl | H | acetyl | H | H | H | H | acetyl | H | H | acetyl |
| 6354 | H | acetyl | H | acetyl | H | H | H | H | acetyl | H | acetyl | H |
| 6355 | H | acetyl | H | acetyl | H | H | H | H | acetyl | H | acetyl | acetyl |
| 6356 | H | acetyl | H | acetyl | H | H | H | H | acetyl | acetyl | H | H |
| 6357 | H | acetyl | H | acetyl | H | H | H | H | acetyl | acetyl | H | acetyl |
| 6358 | H | acetyl | H | acetyl | H | H | H | H | acetyl | acetyl | acetyl | H |
| 6359 | H | acetyl | H | acetyl | H | H | H | H | acetyl | acetyl | acetyl | acetyl |
| 6360 | H | acetyl | H | acetyl | H | H | H | acetyl | H | H | H | H |
| 6361 | H | acetyl | H | acetyl | H | H | H | acetyl | H | H | H | acetyl |
| 6362 | H | acetyl | H | acetyl | H | H | H | acetyl | H | H | acetyl | H |
| 6363 | H | acetyl | H | acetyl | H | H | H | acetyl | H | H | acetyl | acetyl |
| 6364 | H | acetyl | H | acetyl | H | H | H | acetyl | H | acetyl | H | H |
| 6365 | H | acetyl | H | acetyl | H | H | H | acetyl | H | acetyl | H | acetyl |
| 6366 | H | acetyl | H | acetyl | H | H | H | acetyl | H | acetyl | acetyl | H |
| 6367 | H | acetyl | H | acetyl | H | H | H | acetyl | H | acetyl | acetyl | acetyl |
| 6368 | H | acetyl | H | acetyl | H | H | H | acetyl | acetyl | H | H | H |
| 6369 | H | acetyl | H | acetyl | H | H | H | acetyl | acetyl | H | H | acetyl |
| 6370 | H | acetyl | H | acetyl | H | H | H | acetyl | acetyl | H | acetyl | H |
| 6371 | H | acetyl | H | acetyl | H | H | H | acetyl | acetyl | H | acetyl | acetyl |
| 6372 | H | acetyl | H | acetyl | H | H | H | acetyl | acetyl | acetyl | H | H |
| 6373 | H | acetyl | H | acetyl | H | H | H | acetyl | acetyl | acetyl | H | acetyl |
| 6374 | H | acetyl | H | acetyl | H | H | H | acetyl | acetyl | acetyl | acetyl | H |
| 6375 | H | acetyl | H | acetyl | H | H | H | acetyl | acetyl | acetyl | acetyl | acetyl |
| 6376 | H | acetyl | H | acetyl | H | H | acetyl | H | H | H | H | H |
| 6377 | H | acetyl | H | acetyl | H | H | acetyl | H | H | H | H | acetyl |
| 6378 | H | acetyl | H | acetyl | H | H | acetyl | H | H | H | acetyl | H |
| 6379 | H | acetyl | H | acetyl | H | H | acetyl | H | H | H | acetyl | acetyl |
| 6380 | H | acetyl | H | acetyl | H | H | acetyl | H | H | acetyl | H | H |
| 6381 | H | acetyl | H | acetyl | H | H | acetyl | H | H | acetyl | H | acetyl |
| 6382 | H | acetyl | H | acetyl | H | H | acetyl | H | H | acetyl | acetyl | H |
| 6383 | H | acetyl | H | acetyl | H | H | acetyl | H | H | acetyl | acetyl | acetyl |
| 6384 | H | acetyl | H | acetyl | H | H | acetyl | H | acetyl | H | H | H |
| 6385 | H | acetyl | H | acetyl | H | H | acetyl | H | acetyl | H | H | acetyl |
| 6386 | H | acetyl | H | acetyl | H | H | acetyl | H | acetyl | H | acetyl | H |
| 6387 | H | acetyl | H | acetyl | H | H | acetyl | H | acetyl | H | acetyl | acetyl |
| 6388 | H | acetyl | H | acetyl | H | H | acetyl | H | acetyl | acetyl | H | H |
| 6389 | H | acetyl | H | acetyl | H | H | acetyl | H | acetyl | acetyl | H | acetyl |
| 6390 | H | acetyl | H | acetyl | H | H | acetyl | H | acetyl | acetyl | acetyl | H |
| 6391 | H | acetyl | H | acetyl | H | H | acetyl | H | acetyl | acetyl | acetyl | acetyl |
| 6392 | H | acetyl | H | acetyl | H | H | acetyl | acetyl | H | H | H | H |
| 6393 | H | acetyl | H | acetyl | H | H | acetyl | acetyl | H | H | H | acetyl |
| 6394 | H | acetyl | H | acetyl | H | H | acetyl | acetyl | H | H | acetyl | H |
| 6395 | H | acetyl | H | acetyl | H | H | acetyl | acetyl | H | H | acetyl | acetyl |
| 6396 | H | acetyl | H | acetyl | H | H | acetyl | acetyl | H | acetyl | H | H |
| 6397 | H | acetyl | H | acetyl | H | H | acetyl | acetyl | H | acetyl | H | acetyl |
| 6398 | H | acetyl | H | acetyl | H | H | acetyl | acetyl | H | acetyl | acetyl | H |
| 6399 | H | acetyl | H | acetyl | H | H | acetyl | acetyl | H | acetyl | acetyl | acetyl |
| 6400 | H | acetyl | H | acetyl | H | H | acetyl | acetyl | acetyl | H | H | H |
| 6401 | H | acetyl | H | acetyl | H | H | acetyl | acetyl | acetyl | H | H | acetyl |
| 6402 | H | acetyl | H | acetyl | H | H | acetyl | acetyl | acetyl | H | acetyl | H |
| 6403 | H | acetyl | H | acetyl | H | H | acetyl | acetyl | acetyl | H | acetyl | acetyl |
| 6404 | H | acetyl | H | acetyl | H | H | acetyl | acetyl | acetyl | acetyl | H | H |
| 6405 | H | acetyl | H | acetyl | H | H | acetyl | acetyl | acetyl | acetyl | H | acetyl |
| 6406 | H | acetyl | H | acetyl | H | H | acetyl | acetyl | acetyl | acetyl | acetyl | H |
| 6407 | H | acetyl | H | acetyl | H | H | acetyl | acetyl | acetyl | acetyl | acetyl | acetyl |
| 6408 | H | acetyl | H | acetyl | H | acetyl | H | H | H | H | H | H |
| 6409 | H | acetyl | H | acetyl | H | acetyl | H | H | H | H | H | acetyl |
| 6410 | H | acetyl | H | acetyl | H | acetyl | H | H | H | H | acetyl | H |
| 6411 | H | acetyl | H | acetyl | H | acetyl | H | H | H | H | acetyl | acetyl |
| 6412 | H | acetyl | H | acetyl | H | acetyl | H | H | H | acetyl | H | H |
| 6413 | H | acetyl | H | acetyl | H | acetyl | H | H | H | acetyl | H | acetyl |
| 6414 | H | acetyl | H | acetyl | H | acetyl | H | H | H | acetyl | acetyl | H |
| 6415 | H | acetyl | H | acetyl | H | acetyl | H | H | H | acetyl | acetyl | acetyl |
| 6416 | H | acetyl | H | acetyl | H | acetyl | H | H | acetyl | H | H | H |
| 6417 | H | acetyl | H | acetyl | H | acetyl | H | H | acetyl | H | H | acetyl |
| 6418 | H | acetyl | H | acetyl | H | acetyl | H | H | acetyl | H | acetyl | H |
| 6419 | H | acetyl | H | acetyl | H | acetyl | H | H | acetyl | H | acetyl | acetyl |
| 6420 | H | acetyl | H | acetyl | H | acetyl | H | H | acetyl | acetyl | H | H |
| 6421 | H | acetyl | H | acetyl | H | acetyl | H | H | acetyl | acetyl | H | acetyl |
| 6422 | H | acetyl | H | acetyl | H | acetyl | H | H | acetyl | acetyl | acetyl | H |
| 6423 | H | acetyl | H | acetyl | H | acetyl | H | H | acetyl | acetyl | acetyl | acetyl |
| 6424 | H | acetyl | H | acetyl | H | acetyl | H | acetyl | H | H | H | H |
| 6425 | H | acetyl | H | acetyl | H | acetyl | H | acetyl | H | H | H | acetyl |
| 6426 | H | acetyl | H | acetyl | H | acetyl | H | acetyl | H | H | acetyl | H |
| 6427 | H | acetyl | H | acetyl | H | acetyl | H | acetyl | H | H | acetyl | acetyl |
| 6428 | H | acetyl | H | acetyl | H | acetyl | H | acetyl | H | acetyl | H | H |
| 6429 | H | acetyl | H | acetyl | H | acetyl | H | acetyl | H | acetyl | H | acetyl |
| 6430 | H | acetyl | H | acetyl | H | acetyl | H | acetyl | H | acetyl | acetyl | H |
| 6431 | H | acetyl | H | acetyl | H | acetyl | H | acetyl | H | acetyl | acetyl | acetyl |
| 6432 | H | acetyl | H | acetyl | H | acetyl | H | acetyl | acetyl | H | H | H |
| 6433 | H | acetyl | H | acetyl | H | acetyl | H | acetyl | acetyl | H | H | acetyl |
| 6434 | H | acetyl | H | acetyl | H | acetyl | H | acetyl | acetyl | H | acetyl | H |
| 6435 | H | acetyl | H | acetyl | H | acetyl | H | acetyl | acetyl | H | acetyl | acetyl |
| 6436 | H | acetyl | H | acetyl | H | acetyl | H | acetyl | acetyl | acetyl | H | H |
| 6437 | H | acetyl | H | acetyl | H | acetyl | H | acetyl | acetyl | acetyl | H | acetyl |
| 6438 | H | acetyl | H | acetyl | H | acetyl | H | acetyl | acetyl | acetyl | acetyl | H |
| 6439 | H | acetyl | H | acetyl | H | acetyl | H | acetyl | acetyl | acetyl | acetyl | acetyl |
| 6440 | H | acetyl | H | acetyl | H | acetyl | acetyl | H | H | H | H | H |
| 6441 | H | acetyl | H | acetyl | H | acetyl | acetyl | H | H | H | H | acetyl |
| 6442 | H | acetyl | H | acetyl | H | acetyl | acetyl | H | H | H | acetyl | H |
| 6443 | H | acetyl | H | acetyl | H | acetyl | acetyl | H | H | H | acetyl | acetyl |
| 6444 | H | acetyl | H | acetyl | H | acetyl | acetyl | H | H | acetyl | H | H |
| 6445 | H | acetyl | H | acetyl | H | acetyl | acetyl | H | H | acetyl | H | acetyl |
| 6446 | H | acetyl | H | acetyl | H | acetyl | acetyl | H | H | acetyl | acetyl | H |
| 6447 | H | acetyl | H | acetyl | H | acetyl | acetyl | H | H | acetyl | acetyl | acetyl |
| 6448 | H | acetyl | H | acetyl | H | acetyl | acetyl | H | acetyl | H | H | H |
| 6449 | H | acetyl | H | acetyl | H | acetyl | acetyl | H | acetyl | H | H | acetyl |
| 6450 | H | acetyl | H | acetyl | H | acetyl | acetyl | H | acetyl | H | acetyl | H |
| 6451 | H | acetyl | H | acetyl | H | acetyl | acetyl | H | acetyl | H | acetyl | acetyl |
| 6452 | H | acetyl | H | acetyl | H | acetyl | acetyl | H | acetyl | acetyl | H | H |
| 6453 | H | acetyl | H | acetyl | H | acetyl | acetyl | H | acetyl | acetyl | H | acetyl |
| 6454 | H | acetyl | H | acetyl | H | acetyl | acetyl | H | acetyl | acetyl | acetyl | H |
| 6455 | H | acetyl | H | acetyl | H | acetyl | acetyl | H | acetyl | acetyl | acetyl | acetyl |
| 6456 | H | acetyl | H | acetyl | H | acetyl | acetyl | acetyl | H | H | H | H |
| 6457 | H | acetyl | H | acetyl | H | acetyl | acetyl | acetyl | H | H | H | acetyl |
| 6458 | H | acetyl | H | acetyl | H | acetyl | acetyl | acetyl | H | H | acetyl | H |
| 6459 | H | acetyl | H | acetyl | H | acetyl | acetyl | acetyl | H | H | acetyl | acetyl |
| 6460 | H | acetyl | H | acetyl | H | acetyl | acetyl | acetyl | H | acetyl | H | H |
| 6461 | H | acetyl | H | acetyl | H | acetyl | acetyl | acetyl | H | acetyl | H | acetyl |
| 6462 | H | acetyl | H | acetyl | H | acetyl | acetyl | acetyl | H | acetyl | acetyl | H |
| 6463 | H | acetyl | H | acetyl | H | acetyl | acetyl | acetyl | H | acetyl | acetyl | acetyl |
| 6464 | H | acetyl | H | acetyl | H | acetyl | acetyl | acetyl | acetyl | H | H | H |
| 6465 | H | acetyl | H | acetyl | H | acetyl | acetyl | acetyl | acetyl | H | H | acetyl |
| 6466 | H | acetyl | H | acetyl | H | acetyl | acetyl | acetyl | acetyl | H | acetyl | H |
| 6467 | H | acetyl | H | acetyl | H | acetyl | acetyl | acetyl | acetyl | H | acetyl | acetyl |
| 6468 | H | acetyl | H | acetyl | H | acetyl | acetyl | acetyl | acetyl | acetyl | H | H |
| 6469 | H | acetyl | H | acetyl | H | acetyl | acetyl | acetyl | acetyl | acetyl | H | acetyl |
| 6470 | H | acetyl | H | acetyl | H | acetyl | acetyl | acetyl | acetyl | acetyl | acetyl | H |
| 6471 | H | acetyl | H | acetyl | H | acetyl | acetyl | acetyl | acetyl | acetyl | acetyl | acetyl |
| 6472 | H | acetyl | H | acetyl | acetyl | H | H | H | H | H | H | H |
| 6473 | H | acetyl | H | acetyl | acetyl | H | H | H | H | H | H | acetyl |
| 6474 | H | acetyl | H | acetyl | acetyl | H | H | H | H | H | acetyl | H |
| 6475 | H | acetyl | H | acetyl | acetyl | H | H | H | H | H | acetyl | acetyl |
| 6476 | H | acetyl | H | acetyl | acetyl | H | H | H | H | acetyl | H | H |
| 6477 | H | acetyl | H | acetyl | acetyl | H | H | H | H | acetyl | H | acetyl |
| 6478 | H | acetyl | H | acetyl | acetyl | H | H | H | H | acetyl | acetyl | H |
| 6479 | H | acetyl | H | acetyl | acetyl | H | H | H | H | acetyl | acetyl | acetyl |
| 6480 | H | acetyl | H | acetyl | acetyl | H | H | H | acetyl | H | H | H |
| 6481 | H | acetyl | H | acetyl | acetyl | H | H | H | acetyl | H | H | acetyl |
| 6482 | H | acetyl | H | acetyl | acetyl | H | H | H | acetyl | H | acetyl | H |
| 6483 | H | acetyl | H | acetyl | acetyl | H | H | H | acetyl | H | acetyl | acetyl |
| 6484 | H | acetyl | H | acetyl | acetyl | H | H | H | acetyl | acetyl | H | H |
| 6485 | H | acetyl | H | acetyl | acetyl | H | H | H | acetyl | acetyl | H | acetyl |
| 6486 | H | acetyl | H | acetyl | acetyl | H | H | H | acetyl | acetyl | acetyl | H |
| 6487 | H | acetyl | H | acetyl | acetyl | H | H | H | acetyl | acetyl | acetyl | acetyl |
| 6488 | H | acetyl | H | acetyl | acetyl | H | H | acetyl | H | H | H | H |
| 6489 | H | acetyl | H | acetyl | acetyl | H | H | acetyl | H | H | H | acetyl |
| 6490 | H | acetyl | H | acetyl | acetyl | H | H | acetyl | H | H | acetyl | H |
| 6491 | H | acetyl | H | acetyl | acetyl | H | H | acetyl | H | H | acetyl | acetyl |
| 6492 | H | acetyl | H | acetyl | acetyl | H | H | acetyl | H | acetyl | H | H |
| 6493 | H | acetyl | H | acetyl | acetyl | H | H | acetyl | H | acetyl | H | acetyl |
| 6494 | H | acetyl | H | acetyl | acetyl | H | H | acetyl | H | acetyl | acetyl | H |
| 6495 | H | acetyl | H | acetyl | acetyl | H | H | acetyl | H | acetyl | acetyl | acetyl |
| 6496 | H | acetyl | H | acetyl | acetyl | H | H | acetyl | acetyl | H | H | H |
| 6497 | H | acetyl | H | acetyl | acetyl | H | H | acetyl | acetyl | H | H | acetyl |
| 6498 | H | acetyl | H | acetyl | acetyl | H | H | acetyl | acetyl | H | acetyl | H |
| 6499 | H | acetyl | H | acetyl | acetyl | H | H | acetyl | acetyl | H | acetyl | acetyl |
| 6500 | H | acetyl | H | acetyl | acetyl | H | H | acetyl | acetyl | acetyl | H | H |
| 6501 | H | acetyl | H | acetyl | acetyl | H | H | acetyl | acetyl | acetyl | H | acetyl |
| 6502 | H | acetyl | H | acetyl | acetyl | H | H | acetyl | acetyl | acetyl | acetyl | H |
| 6503 | H | acetyl | H | acetyl | acetyl | H | H | acetyl | acetyl | acetyl | acetyl | acetyl |
| 6504 | H | acetyl | H | acetyl | acetyl | H | acetyl | H | H | H | H | H |
| 6505 | H | acetyl | H | acetyl | acetyl | H | acetyl | H | H | H | H | acetyl |
| 6506 | H | acetyl | H | acetyl | acetyl | H | acetyl | H | H | H | acetyl | H |
| 6507 | H | acetyl | H | acetyl | acetyl | H | acetyl | H | H | H | acetyl | acetyl |
| 6508 | H | acetyl | H | acetyl | acetyl | H | acetyl | H | H | acetyl | H | H |
| 6509 | H | acetyl | H | acetyl | acetyl | H | acetyl | H | H | acetyl | H | acetyl |
| 6510 | H | acetyl | H | acetyl | acetyl | H | acetyl | H | H | acetyl | acetyl | H |
| 6511 | H | acetyl | H | acetyl | acetyl | H | acetyl | H | H | acetyl | acetyl | acetyl |
| 6512 | H | acetyl | H | acetyl | acetyl | H | acetyl | H | acetyl | H | H | H |
| 6513 | H | acetyl | H | acetyl | acetyl | H | acetyl | H | acetyl | H | H | acetyl |
| 6514 | H | acetyl | H | acetyl | acetyl | H | acetyl | H | acetyl | H | acetyl | H |
| 6515 | H | acetyl | H | acetyl | acetyl | H | acetyl | H | acetyl | H | acetyl | acetyl |
| 6516 | H | acetyl | H | acetyl | acetyl | H | acetyl | H | acetyl | acetyl | H | H |
| 6517 | H | acetyl | H | acetyl | acetyl | H | acetyl | H | acetyl | acetyl | H | acetyl |
| 6518 | H | acetyl | H | acetyl | acetyl | H | acetyl | H | acetyl | acetyl | acetyl | H |
| 6519 | H | acetyl | H | acetyl | acetyl | H | acetyl | H | acetyl | acetyl | acetyl | acetyl |
| 6520 | H | acetyl | H | acetyl | acetyl | H | acetyl | acetyl | H | H | H | H |
| 6521 | H | acetyl | H | acetyl | acetyl | H | acetyl | acetyl | H | H | H | acetyl |
| 6522 | H | acetyl | H | acetyl | acetyl | H | acetyl | acetyl | H | H | acetyl | H |
| 6523 | H | acetyl | H | acetyl | acetyl | H | acetyl | acetyl | H | H | acetyl | acetyl |
| 6524 | H | acetyl | H | acetyl | acetyl | H | acetyl | acetyl | H | acetyl | H | H |
| 6525 | H | acetyl | H | acetyl | acetyl | H | acetyl | acetyl | H | acetyl | H | acetyl |
| 6526 | H | acetyl | H | acetyl | acetyl | H | acetyl | acetyl | H | acetyl | acetyl | H |
| 6527 | H | acetyl | H | acetyl | acetyl | H | acetyl | acetyl | H | acetyl | acetyl | acetyl |
| 6528 | H | acetyl | H | acetyl | acetyl | H | acetyl | acetyl | acetyl | H | H | H |
| 6529 | H | acetyl | H | acetyl | acetyl | H | acetyl | acetyl | acetyl | H | H | acetyl |
| 6530 | H | acetyl | H | acetyl | acetyl | H | acetyl | acetyl | acetyl | H | acetyl | H |
| 6531 | H | acetyl | H | acetyl | acetyl | H | acetyl | acetyl | acetyl | H | acetyl | acetyl |
| 6532 | H | acetyl | H | acetyl | acetyl | H | acetyl | acetyl | acetyl | acetyl | H | H |
| 6533 | H | acetyl | H | acetyl | acetyl | H | acetyl | acetyl | acetyl | acetyl | H | acetyl |
| 6534 | H | acetyl | H | acetyl | acetyl | H | acetyl | acetyl | acetyl | acetyl | acetyl | H |
| 6535 | H | acetyl | H | acetyl | acetyl | H | acetyl | acetyl | acetyl | acetyl | acetyl | acetyl |
| 6536 | H | acetyl | H | acetyl | acetyl | acetyl | H | H | H | H | H | H |
| 6537 | H | acetyl | H | acetyl | acetyl | acetyl | H | H | H | H | H | acetyl |
| 6538 | H | acetyl | H | acetyl | acetyl | acetyl | H | H | H | H | acetyl | H |
| 6539 | H | acetyl | H | acetyl | acetyl | acetyl | H | H | H | H | acetyl | acetyl |
| 6540 | H | acetyl | H | acetyl | acetyl | acetyl | H | H | H | acetyl | H | H |
| 6541 | H | acetyl | H | acetyl | acetyl | acetyl | H | H | H | acetyl | H | acetyl |
| 6542 | H | acetyl | H | acetyl | acetyl | acetyl | H | H | H | acetyl | acetyl | H |
| 6543 | H | acetyl | H | acetyl | acetyl | acetyl | H | H | H | acetyl | acetyl | acetyl |
| 6544 | H | acetyl | H | acetyl | acetyl | acetyl | H | H | acetyl | H | H | H |
| 6545 | H | acetyl | H | acetyl | acetyl | acetyl | H | H | acetyl | H | H | acetyl |
| 6546 | H | acetyl | H | acetyl | acetyl | acetyl | H | H | acetyl | H | acetyl | H |
| 6547 | H | acetyl | H | acetyl | acetyl | acetyl | H | H | acetyl | H | acetyl | acetyl |
| 6548 | H | acetyl | H | acetyl | acetyl | acetyl | H | H | acetyl | acetyl | H | H |
| 6549 | H | acetyl | H | acetyl | acetyl | acetyl | H | H | acetyl | acetyl | H | acetyl |
| 6550 | H | acetyl | H | acetyl | acetyl | acetyl | H | H | acetyl | acetyl | acetyl | H |
| 6551 | H | acetyl | H | acetyl | acetyl | acetyl | H | H | acetyl | acetyl | acetyl | acetyl |
| 6552 | H | acetyl | H | acetyl | acetyl | acetyl | H | acetyl | H | H | H | H |
| 6553 | H | acetyl | H | acetyl | acetyl | acetyl | H | acetyl | H | H | H | acetyl |
| 6554 | H | acetyl | H | acetyl | acetyl | acetyl | H | acetyl | H | H | acetyl | H |
| 6555 | H | acetyl | H | acetyl | acetyl | acetyl | H | acetyl | H | H | acetyl | acetyl |
| 6556 | H | acetyl | H | acetyl | acetyl | acetyl | H | acetyl | H | acetyl | H | H |
| 6557 | H | acetyl | H | acetyl | acetyl | acetyl | H | acetyl | H | acetyl | H | acetyl |
| 6558 | H | acetyl | H | acetyl | acetyl | acetyl | H | acetyl | H | acetyl | acetyl | H |
| 6559 | H | acetyl | H | acetyl | acetyl | acetyl | H | acetyl | H | acetyl | acetyl | acetyl |
| 6560 | H | acetyl | H | acetyl | acetyl | acetyl | H | acetyl | acetyl | H | H | H |
| 6561 | H | acetyl | H | acetyl | acetyl | acetyl | H | acetyl | acetyl | H | H | acetyl |
| 6562 | H | acetyl | H | acetyl | acetyl | acetyl | H | acetyl | acetyl | H | acetyl | H |
| 6563 | H | acetyl | H | acetyl | acetyl | acetyl | H | acetyl | acetyl | H | acetyl | acetyl |
| 6564 | H | acetyl | H | acetyl | acetyl | acetyl | H | acetyl | acetyl | acetyl | H | H |
| 6565 | H | acetyl | H | acetyl | acetyl | acetyl | H | acetyl | acetyl | acetyl | H | acetyl |
| 6566 | H | acetyl | H | acetyl | acetyl | acetyl | H | acetyl | acetyl | acetyl | acetyl | H |
| 6567 | H | acetyl | H | acetyl | acetyl | acetyl | H | acetyl | acetyl | acetyl | acetyl | acetyl |
| 6568 | H | acetyl | H | acetyl | acetyl | acetyl | acetyl | H | H | H | H | H |
| 6569 | H | acetyl | H | acetyl | acetyl | acetyl | acetyl | H | H | H | H | acetyl |
| 6570 | H | acetyl | H | acetyl | acetyl | acetyl | acetyl | H | H | H | acetyl | H |
| 6571 | H | acetyl | H | acetyl | acetyl | acetyl | acetyl | H | H | H | acetyl | acetyl |
| 6572 | H | acetyl | H | acetyl | acetyl | acetyl | acetyl | H | H | acetyl | H | H |
| 6573 | H | acetyl | H | acetyl | acetyl | acetyl | acetyl | H | H | acetyl | H | acetyl |
| 6574 | H | acetyl | H | acetyl | acetyl | acetyl | acetyl | H | H | acetyl | acetyl | H |
| 6575 | H | acetyl | H | acetyl | acetyl | acetyl | acetyl | H | H | acetyl | acetyl | acetyl |
| 6576 | H | acetyl | H | acetyl | acetyl | acetyl | acetyl | H | acetyl | H | H | H |
| 6577 | H | acetyl | H | acetyl | acetyl | acetyl | acetyl | H | acetyl | H | H | acetyl |
| 6578 | H | acetyl | H | acetyl | acetyl | acetyl | acetyl | H | acetyl | H | acetyl | H |
| 6579 | H | acetyl | H | acetyl | acetyl | acetyl | acetyl | H | acetyl | H | acetyl | acetyl |
| 6580 | H | acetyl | H | acetyl | acetyl | acetyl | acetyl | H | acetyl | acetyl | H | H |
| 6581 | H | acetyl | H | acetyl | acetyl | acetyl | acetyl | H | acetyl | acetyl | H | acetyl |
| 6582 | H | acetyl | H | acetyl | acetyl | acetyl | acetyl | H | acetyl | acetyl | acetyl | H |
| 6583 | H | acetyl | H | acetyl | acetyl | acetyl | acetyl | H | acetyl | acetyl | acetyl | acetyl |
| 6584 | H | acetyl | H | acetyl | acetyl | acetyl | acetyl | acetyl | H | H | H | H |
| 6585 | H | acetyl | H | acetyl | acetyl | acetyl | acetyl | acetyl | H | H | H | acetyl |
| 6586 | H | acetyl | H | acetyl | acetyl | acetyl | acetyl | acetyl | H | H | acetyl | H |
| 6587 | H | acetyl | H | acetyl | acetyl | acetyl | acetyl | acetyl | H | H | acetyl | acetyl |
| 6588 | H | acetyl | H | acetyl | acetyl | acetyl | acetyl | acetyl | H | acetyl | H | H |
| 6589 | H | acetyl | H | acetyl | acetyl | acetyl | acetyl | acetyl | H | acetyl | H | acetyl |
| 6590 | H | acetyl | H | acetyl | acetyl | acetyl | acetyl | acetyl | H | acetyl | acetyl | H |
| 6591 | H | acetyl | H | acetyl | acetyl | acetyl | acetyl | acetyl | H | acetyl | acetyl | acetyl |
| 6592 | H | acetyl | H | acetyl | acetyl | acetyl | acetyl | acetyl | acetyl | H | H | H |
| 6593 | H | acetyl | H | acetyl | acetyl | acetyl | acetyl | acetyl | acetyl | H | H | acetyl |
| 6594 | H | acetyl | H | acetyl | acetyl | acetyl | acetyl | acetyl | acetyl | H | acetyl | H |
| 6595 | H | acetyl | H | acetyl | acetyl | acetyl | acetyl | acetyl | acetyl | H | acetyl | acetyl |
| 6596 | H | acetyl | H | acetyl | acetyl | acetyl | acetyl | acetyl | acetyl | acetyl | H | H |
| 6597 | H | acetyl | H | acetyl | acetyl | acetyl | acetyl | acetyl | acetyl | acetyl | H | acetyl |
| 6598 | H | acetyl | H | acetyl | acetyl | acetyl | acetyl | acetyl | acetyl | acetyl | acetyl | H |
| 6599 | H | acetyl | H | acetyl | acetyl | acetyl | acetyl | acetyl | acetyl | acetyl | acetyl | acetyl |
| 6600 | H | acetyl | acetyl | H | H | H | H | H | H | H | H | H |
| 6601 | H | acetyl | acetyl | H | H | H | H | H | H | H | H | acetyl |
| 6602 | H | acetyl | acetyl | H | H | H | H | H | H | H | acetyl | H |
| 6603 | H | acetyl | acetyl | H | H | H | H | H | H | H | acetyl | acetyl |
| 6604 | H | acetyl | acetyl | H | H | H | H | H | H | acetyl | H | H |
| 6605 | H | acetyl | acetyl | H | H | H | H | H | H | acetyl | H | acetyl |
| 6606 | H | acetyl | acetyl | H | H | H | H | H | H | acetyl | acetyl | H |
| 6607 | H | acetyl | acetyl | H | H | H | H | H | H | acetyl | acetyl | acetyl |
| 6608 | H | acetyl | acetyl | H | H | H | H | H | acetyl | H | H | H |
| 6609 | H | acetyl | acetyl | H | H | H | H | H | acetyl | H | H | acetyl |
| 6610 | H | acetyl | acetyl | H | H | H | H | H | acetyl | H | acetyl | H |
| 6611 | H | acetyl | acetyl | H | H | H | H | H | acetyl | H | acetyl | acetyl |
| 6612 | H | acetyl | acetyl | H | H | H | H | H | acetyl | acetyl | H | H |
| 6613 | H | acetyl | acetyl | H | H | H | H | H | acetyl | acetyl | H | acetyl |
| 6614 | H | acetyl | acetyl | H | H | H | H | H | acetyl | acetyl | acetyl | H |
| 6615 | H | acetyl | acetyl | H | H | H | H | H | acetyl | acetyl | acetyl | acetyl |
| 6616 | H | acetyl | acetyl | H | H | H | H | acetyl | H | H | H | H |
| 6617 | H | acetyl | acetyl | H | H | H | H | acetyl | H | H | H | acetyl |
| 6618 | H | acetyl | acetyl | H | H | H | H | acetyl | H | H | acetyl | H |
| 6619 | H | acetyl | acetyl | H | H | H | H | acetyl | H | H | acetyl | acetyl |
| 6620 | H | acetyl | acetyl | H | H | H | H | acetyl | H | acetyl | H | H |
| 6621 | H | acetyl | acetyl | H | H | H | H | acetyl | H | acetyl | H | acetyl |
| 6622 | H | acetyl | acetyl | H | H | H | H | acetyl | H | acetyl | acetyl | H |
| 6623 | H | acetyl | acetyl | H | H | H | H | acetyl | H | acetyl | acetyl | acetyl |
| 6624 | H | acetyl | acetyl | H | H | H | H | acetyl | acetyl | H | H | H |
| 6625 | H | acetyl | acetyl | H | H | H | H | acetyl | acetyl | H | H | acetyl |
| 6626 | H | acetyl | acetyl | H | H | H | H | acetyl | acetyl | H | acetyl | H |
| 6627 | H | acetyl | acetyl | H | H | H | H | acetyl | acetyl | H | acetyl | acetyl |
| 6628 | H | acetyl | acetyl | H | H | H | H | acetyl | acetyl | acetyl | H | H |
| 6629 | H | acetyl | acetyl | H | H | H | H | acetyl | acetyl | acetyl | H | acetyl |
| 6630 | H | acetyl | acetyl | H | H | H | H | acetyl | acetyl | acetyl | acetyl | H |
| 6631 | H | acetyl | acetyl | H | H | H | H | acetyl | acetyl | acetyl | acetyl | acetyl |
| 6632 | H | acetyl | acetyl | H | H | H | acetyl | H | H | H | H | H |
| 6633 | H | acetyl | acetyl | H | H | H | acetyl | H | H | H | H | acetyl |
| 6634 | H | acetyl | acetyl | H | H | H | acetyl | H | H | H | acetyl | H |
| 6635 | H | acetyl | acetyl | H | H | H | acetyl | H | H | H | acetyl | acetyl |
| 6636 | H | acetyl | acetyl | H | H | H | acetyl | H | H | acetyl | H | H |
| 6637 | H | acetyl | acetyl | H | H | H | acetyl | H | H | acetyl | H | acetyl |
| 6638 | H | acetyl | acetyl | H | H | H | acetyl | H | H | acetyl | acetyl | H |
| 6639 | H | acetyl | acetyl | H | H | H | acetyl | H | H | acetyl | acetyl | acetyl |
| 6640 | H | acetyl | acetyl | H | H | H | acetyl | H | acetyl | H | H | H |
| 6641 | H | acetyl | acetyl | H | H | H | acetyl | H | acetyl | H | H | acetyl |
| 6642 | H | acetyl | acetyl | H | H | H | acetyl | H | acetyl | H | acetyl | H |
| 6643 | H | acetyl | acetyl | H | H | H | acetyl | H | acetyl | H | acetyl | acetyl |
| 6644 | H | acetyl | acetyl | H | H | H | acetyl | H | acetyl | acetyl | H | H |
| 6645 | H | acetyl | acetyl | H | H | H | acetyl | H | acetyl | acetyl | H | acetyl |
| 6646 | H | acetyl | acetyl | H | H | H | acetyl | H | acetyl | acetyl | acetyl | H |
| 6647 | H | acetyl | acetyl | H | H | H | acetyl | H | acetyl | acetyl | acetyl | acetyl |
| 6648 | H | acetyl | acetyl | H | H | H | acetyl | acetyl | H | H | H | H |
| 6649 | H | acetyl | acetyl | H | H | H | acetyl | acetyl | H | H | H | acetyl |
| 6650 | H | acetyl | acetyl | H | H | H | acetyl | acetyl | H | H | acetyl | H |
| 6651 | H | acetyl | acetyl | H | H | H | acetyl | acetyl | H | H | acetyl | acetyl |
| 6652 | H | acetyl | acetyl | H | H | H | acetyl | acetyl | H | acetyl | H | H |
| 6653 | H | acetyl | acetyl | H | H | H | acetyl | acetyl | H | acetyl | H | acetyl |
| 6654 | H | acetyl | acetyl | H | H | H | acetyl | acetyl | H | acetyl | acetyl | H |
| 6655 | H | acetyl | acetyl | H | H | H | acetyl | acetyl | H | acetyl | acetyl | acetyl |
| 6656 | H | acetyl | acetyl | H | H | H | acetyl | acetyl | acetyl | H | H | H |
| 6657 | H | acetyl | acetyl | H | H | H | acetyl | acetyl | acetyl | H | H | acetyl |
| 6658 | H | acetyl | acetyl | H | H | H | acetyl | acetyl | acetyl | H | acetyl | H |
| 6659 | H | acetyl | acetyl | H | H | H | acetyl | acetyl | acetyl | H | acetyl | acetyl |
| 6660 | H | acetyl | acetyl | H | H | H | acetyl | acetyl | acetyl | acetyl | H | H |
| 6661 | H | acetyl | acetyl | H | H | H | acetyl | acetyl | acetyl | acetyl | H | acetyl |
| 6662 | H | acetyl | acetyl | H | H | H | acetyl | acetyl | acetyl | acetyl | acetyl | H |
| 6663 | H | acetyl | acetyl | H | H | H | acetyl | acetyl | acetyl | acetyl | acetyl | acetyl |
| 6664 | H | acetyl | acetyl | H | H | acetyl | H | H | H | H | H | H |
| 6665 | H | acetyl | acetyl | H | H | acetyl | H | H | H | H | H | acetyl |
| 6666 | H | acetyl | acetyl | H | H | acetyl | H | H | H | H | acetyl | H |
| 6667 | H | acetyl | acetyl | H | H | acetyl | H | H | H | H | acetyl | acetyl |
| 6668 | H | acetyl | acetyl | H | H | acetyl | H | H | H | acetyl | H | H |
| 6669 | H | acetyl | acetyl | H | H | acetyl | H | H | H | acetyl | H | acetyl |
| 6670 | H | acetyl | acetyl | H | H | acetyl | H | H | H | acetyl | acetyl | H |
| 6671 | H | acetyl | acetyl | H | H | acetyl | H | H | H | acetyl | acetyl | acetyl |
| 6672 | H | acetyl | acetyl | H | H | acetyl | H | H | acetyl | H | H | H |
| 6673 | H | acetyl | acetyl | H | H | acetyl | H | H | acetyl | H | H | acetyl |
| 6674 | H | acetyl | acetyl | H | H | acetyl | H | H | acetyl | H | acetyl | H |
| 6675 | H | acetyl | acetyl | H | H | acetyl | H | H | acetyl | H | acetyl | acetyl |
| 6676 | H | acetyl | acetyl | H | H | acetyl | H | H | acetyl | acetyl | H | H |
| 6677 | H | acetyl | acetyl | H | H | acetyl | H | H | acetyl | acetyl | H | acetyl |
| 6678 | H | acetyl | acetyl | H | H | acetyl | H | H | acetyl | acetyl | acetyl | H |
| 6679 | H | acetyl | acetyl | H | H | acetyl | H | H | acetyl | acetyl | acetyl | acetyl |
| 6680 | H | acetyl | acetyl | H | H | acetyl | H | acetyl | H | H | H | H |
| 6681 | H | acetyl | acetyl | H | H | acetyl | H | acetyl | H | H | H | acetyl |
| 6682 | H | acetyl | acetyl | H | H | acetyl | H | acetyl | H | H | acetyl | H |
| 6683 | H | acetyl | acetyl | H | H | acetyl | H | acetyl | H | H | acetyl | acetyl |
| 6684 | H | acetyl | acetyl | H | H | acetyl | H | acetyl | H | acetyl | H | H |
| 6685 | H | acetyl | acetyl | H | H | acetyl | H | acetyl | H | acetyl | H | acetyl |
| 6686 | H | acetyl | acetyl | H | H | acetyl | H | acetyl | H | acetyl | acetyl | H |
| 6687 | H | acetyl | acetyl | H | H | acetyl | H | acetyl | H | acetyl | acetyl | acetyl |
| 6688 | H | acetyl | acetyl | H | H | acetyl | H | acetyl | acetyl | H | H | H |
| 6689 | H | acetyl | acetyl | H | H | acetyl | H | acetyl | acetyl | H | H | acetyl |
| 6690 | H | acetyl | acetyl | H | H | acetyl | H | acetyl | acetyl | H | acetyl | H |
| 6691 | H | acetyl | acetyl | H | H | acetyl | H | acetyl | acetyl | H | acetyl | acetyl |
| 6692 | H | acetyl | acetyl | H | H | acetyl | H | acetyl | acetyl | acetyl | H | H |
| 6693 | H | acetyl | acetyl | H | H | acetyl | H | acetyl | acetyl | acetyl | H | acetyl |
| 6694 | H | acetyl | acetyl | H | H | acetyl | H | acetyl | acetyl | acetyl | acetyl | H |
| 6695 | H | acetyl | acetyl | H | H | acetyl | H | acetyl | acetyl | acetyl | acetyl | acetyl |
| 6696 | H | acetyl | acetyl | H | H | acetyl | acetyl | H | H | H | H | H |
| 6697 | H | acetyl | acetyl | H | H | acetyl | acetyl | H | H | H | H | acetyl |
| 6698 | H | acetyl | acetyl | H | H | acetyl | acetyl | H | H | H | acetyl | H |
| 6699 | H | acetyl | acetyl | H | H | acetyl | acetyl | H | H | H | acetyl | acetyl |
| 6700 | H | acetyl | acetyl | H | H | acetyl | acetyl | H | H | acetyl | H | H |
| 6701 | H | acetyl | acetyl | H | H | acetyl | acetyl | H | H | acetyl | H | acetyl |
| 6702 | H | acetyl | acetyl | H | H | acetyl | acetyl | H | H | acetyl | acetyl | H |
| 6703 | H | acetyl | acetyl | H | H | acetyl | acetyl | H | H | acetyl | acetyl | acetyl |
| 6704 | H | acetyl | acetyl | H | H | acetyl | acetyl | H | acetyl | H | H | H |
| 6705 | H | acetyl | acetyl | H | H | acetyl | acetyl | H | acetyl | H | H | acetyl |
| 6706 | H | acetyl | acetyl | H | H | acetyl | acetyl | H | acetyl | H | acetyl | H |
| 6707 | H | acetyl | acetyl | H | H | acetyl | acetyl | H | acetyl | H | acetyl | acetyl |
| 6708 | H | acetyl | acetyl | H | H | acetyl | acetyl | H | acetyl | acetyl | H | H |
| 6709 | H | acetyl | acetyl | H | H | acetyl | acetyl | H | acetyl | acetyl | H | acetyl |
| 6710 | H | acetyl | acetyl | H | H | acetyl | acetyl | H | acetyl | acetyl | acetyl | H |
| 6711 | H | acetyl | acetyl | H | H | acetyl | acetyl | H | acetyl | acetyl | acetyl | acetyl |
| 6712 | H | acetyl | acetyl | H | H | acetyl | acetyl | acetyl | H | H | H | H |
| 6713 | H | acetyl | acetyl | H | H | acetyl | acetyl | acetyl | H | H | H | acetyl |
| 6714 | H | acetyl | acetyl | H | H | acetyl | acetyl | acetyl | H | H | acetyl | H |
| 6715 | H | acetyl | acetyl | H | H | acetyl | acetyl | acetyl | H | H | acetyl | acetyl |
| 6716 | H | acetyl | acetyl | H | H | acetyl | acetyl | acetyl | H | acetyl | H | H |
| 6717 | H | acetyl | acetyl | H | H | acetyl | acetyl | acetyl | H | acetyl | H | acetyl |
| 6718 | H | acetyl | acetyl | H | H | acetyl | acetyl | acetyl | H | acetyl | acetyl | H |
| 6719 | H | acetyl | acetyl | H | H | acetyl | acetyl | acetyl | H | acetyl | acetyl | acetyl |
| 6720 | H | acetyl | acetyl | H | H | acetyl | acetyl | acetyl | acetyl | H | H | H |
| 6721 | H | acetyl | acetyl | H | H | acetyl | acetyl | acetyl | acetyl | H | H | acetyl |
| 6722 | H | acetyl | acetyl | H | H | acetyl | acetyl | acetyl | acetyl | H | acetyl | H |
| 6723 | H | acetyl | acetyl | H | H | acetyl | acetyl | acetyl | acetyl | H | acetyl | acetyl |
| 6724 | H | acetyl | acetyl | H | H | acetyl | acetyl | acetyl | acetyl | acetyl | H | H |
| 6725 | H | acetyl | acetyl | H | H | acetyl | acetyl | acetyl | acetyl | acetyl | H | acetyl |
| 6726 | H | acetyl | acetyl | H | H | acetyl | acetyl | acetyl | acetyl | acetyl | acetyl | H |
| 6727 | H | acetyl | acetyl | H | H | acetyl | acetyl | acetyl | acetyl | acetyl | acetyl | acetyl |
| 6728 | H | acetyl | acetyl | H | acetyl | H | H | H | H | H | H | H |
| 6729 | H | acetyl | acetyl | H | acetyl | H | H | H | H | H | H | acetyl |
| 6730 | H | acetyl | acetyl | H | acetyl | H | H | H | H | H | acetyl | H |
| 6731 | H | acetyl | acetyl | H | acetyl | H | H | H | H | H | acetyl | acetyl |
| 6732 | H | acetyl | acetyl | H | acetyl | H | H | H | H | acetyl | H | H |
| 6733 | H | acetyl | acetyl | H | acetyl | H | H | H | H | acetyl | H | acetyl |
| 6734 | H | acetyl | acetyl | H | acetyl | H | H | H | H | acetyl | acetyl | H |
| 6735 | H | acetyl | acetyl | H | acetyl | H | H | H | H | acetyl | acetyl | acetyl |
| 6736 | H | acetyl | acetyl | H | acetyl | H | H | H | acetyl | H | H | H |
| 6737 | H | acetyl | acetyl | H | acetyl | H | H | H | acetyl | H | H | acetyl |
| 6738 | H | acetyl | acetyl | H | acetyl | H | H | H | acetyl | H | acetyl | H |
| 6739 | H | acetyl | acetyl | H | acetyl | H | H | H | acetyl | H | acetyl | acetyl |
| 6740 | H | acetyl | acetyl | H | acetyl | H | H | H | acetyl | acetyl | H | H |
| 6741 | H | acetyl | acetyl | H | acetyl | H | H | H | acetyl | acetyl | H | acetyl |
| 6742 | H | acetyl | acetyl | H | acetyl | H | H | H | acetyl | acetyl | acetyl | H |
| 6743 | H | acetyl | acetyl | H | acetyl | H | H | H | acetyl | acetyl | acetyl | acetyl |
| 6744 | H | acetyl | acetyl | H | acetyl | H | H | acetyl | H | H | H | H |
| 6745 | H | acetyl | acetyl | H | acetyl | H | H | acetyl | H | H | H | acetyl |
| 6746 | H | acetyl | acetyl | H | acetyl | H | H | acetyl | H | H | acetyl | H |
| 6747 | H | acetyl | acetyl | H | acetyl | H | H | acetyl | H | H | acetyl | acetyl |
| 6748 | H | acetyl | acetyl | H | acetyl | H | H | acetyl | H | acetyl | H | H |
| 6749 | H | acetyl | acetyl | H | acetyl | H | H | acetyl | H | acetyl | H | acetyl |
| 6750 | H | acetyl | acetyl | H | acetyl | H | H | acetyl | H | acetyl | acetyl | H |
| 6751 | H | acetyl | acetyl | H | acetyl | H | H | acetyl | H | acetyl | acetyl | acetyl |
| 6752 | H | acetyl | acetyl | H | acetyl | H | H | acetyl | acetyl | H | H | H |
| 6753 | H | acetyl | acetyl | H | acetyl | H | H | acetyl | acetyl | H | H | acetyl |
| 6754 | H | acetyl | acetyl | H | acetyl | H | H | acetyl | acetyl | H | acetyl | H |
| 6755 | H | acetyl | acetyl | H | acetyl | H | H | acetyl | acetyl | H | acetyl | acetyl |
| 6756 | H | acetyl | acetyl | H | acetyl | H | H | acetyl | acetyl | acetyl | H | H |
| 6757 | H | acetyl | acetyl | H | acetyl | H | H | acetyl | acetyl | acetyl | H | acetyl |
| 6758 | H | acetyl | acetyl | H | acetyl | H | H | acetyl | acetyl | acetyl | acetyl | H |
| 6759 | H | acetyl | acetyl | H | acetyl | H | H | acetyl | acetyl | acetyl | acetyl | acetyl |
| 6760 | H | acetyl | acetyl | H | acetyl | H | acetyl | H | H | H | H | H |
| 6761 | H | acetyl | acetyl | H | acetyl | H | acetyl | H | H | H | H | acetyl |
| 6762 | H | acetyl | acetyl | H | acetyl | H | acetyl | H | H | H | acetyl | H |
| 6763 | H | acetyl | acetyl | H | acetyl | H | acetyl | H | H | H | acetyl | acetyl |
| 6764 | H | acetyl | acetyl | H | acetyl | H | acetyl | H | H | acetyl | H | H |
| 6765 | H | acetyl | acetyl | H | acetyl | H | acetyl | H | H | acetyl | H | acetyl |
| 6766 | H | acetyl | acetyl | H | acetyl | H | acetyl | H | H | acetyl | acetyl | H |
| 6767 | H | acetyl | acetyl | H | acetyl | H | acetyl | H | H | acetyl | acetyl | acetyl |
| 6768 | H | acetyl | acetyl | H | acetyl | H | acetyl | H | acetyl | H | H | H |
| 6769 | H | acetyl | acetyl | H | acetyl | H | acetyl | H | acetyl | H | H | acetyl |
| 6770 | H | acetyl | acetyl | H | acetyl | H | acetyl | H | acetyl | H | acetyl | H |
| 6771 | H | acetyl | acetyl | H | acetyl | H | acetyl | H | acetyl | H | acetyl | acetyl |
| 6772 | H | acetyl | acetyl | H | acetyl | H | acetyl | H | acetyl | acetyl | H | H |
| 6773 | H | acetyl | acetyl | H | acetyl | H | acetyl | H | acetyl | acetyl | H | acetyl |
| 6774 | H | acetyl | acetyl | H | acetyl | H | acetyl | H | acetyl | acetyl | acetyl | H |
| 6775 | H | acetyl | acetyl | H | acetyl | H | acetyl | H | acetyl | acetyl | acetyl | acetyl |
| 6776 | H | acetyl | acetyl | H | acetyl | H | acetyl | acetyl | H | H | H | H |
| 6777 | H | acetyl | acetyl | H | acetyl | H | acetyl | acetyl | H | H | H | acetyl |
| 6778 | H | acetyl | acetyl | H | acetyl | H | acetyl | acetyl | H | H | acetyl | H |
| 6779 | H | acetyl | acetyl | H | acetyl | H | acetyl | acetyl | H | H | acetyl | acetyl |
| 6780 | H | acetyl | acetyl | H | acetyl | H | acetyl | acetyl | H | acetyl | H | H |
| 6781 | H | acetyl | acetyl | H | acetyl | H | acetyl | acetyl | H | acetyl | H | acetyl |
| 6782 | H | acetyl | acetyl | H | acetyl | H | acetyl | acetyl | H | acetyl | acetyl | H |
| 6783 | H | acetyl | acetyl | H | acetyl | H | acetyl | acetyl | H | acetyl | acetyl | acetyl |
| 6784 | H | acetyl | acetyl | H | acetyl | H | acetyl | acetyl | acetyl | H | H | H |
| 6785 | H | acetyl | acetyl | H | acetyl | H | acetyl | acetyl | acetyl | H | H | acetyl |
| 6786 | H | acetyl | acetyl | H | acetyl | H | acetyl | acetyl | acetyl | H | acetyl | H |
| 6787 | H | acetyl | acetyl | H | acetyl | H | acetyl | acetyl | acetyl | H | acetyl | acetyl |
| 6788 | H | acetyl | acetyl | H | acetyl | H | acetyl | acetyl | acetyl | acetyl | H | H |
| 6789 | H | acetyl | acetyl | H | acetyl | H | acetyl | acetyl | acetyl | acetyl | H | acetyl |
| 6790 | H | acetyl | acetyl | H | acetyl | H | acetyl | acetyl | acetyl | acetyl | acetyl | H |
| 6791 | H | acetyl | acetyl | H | acetyl | H | acetyl | acetyl | acetyl | acetyl | acetyl | acetyl |
| 6792 | H | acetyl | acetyl | H | acetyl | acetyl | H | H | H | H | H | H |
| 6793 | H | acetyl | acetyl | H | acetyl | acetyl | H | H | H | H | H | acetyl |
| 6794 | H | acetyl | acetyl | H | acetyl | acetyl | H | H | H | H | acetyl | H |
| 6795 | H | acetyl | acetyl | H | acetyl | acetyl | H | H | H | H | acetyl | acetyl |
| 6796 | H | acetyl | acetyl | H | acetyl | acetyl | H | H | H | acetyl | H | H |
| 6797 | H | acetyl | acetyl | H | acetyl | acetyl | H | H | H | acetyl | H | acetyl |
| 6798 | H | acetyl | acetyl | H | acetyl | acetyl | H | H | H | acetyl | acetyl | H |
| 6799 | H | acetyl | acetyl | H | acetyl | acetyl | H | H | H | acetyl | acetyl | acetyl |
| 6800 | H | acetyl | acetyl | H | acetyl | acetyl | H | H | acetyl | H | H | H |
| 6801 | H | acetyl | acetyl | H | acetyl | acetyl | H | H | acetyl | H | H | acetyl |
| 6802 | H | acetyl | acetyl | H | acetyl | acetyl | H | H | acetyl | H | acetyl | H |
| 6803 | H | acetyl | acetyl | H | acetyl | acetyl | H | H | acetyl | H | acetyl | acetyl |
| 6804 | H | acetyl | acetyl | H | acetyl | acetyl | H | H | acetyl | acetyl | H | H |
| 6805 | H | acetyl | acetyl | H | acetyl | acetyl | H | H | acetyl | acetyl | H | acetyl |
| 6806 | H | acetyl | acetyl | H | acetyl | acetyl | H | H | acetyl | acetyl | acetyl | H |
| 6807 | H | acetyl | acetyl | H | acetyl | acetyl | H | H | acetyl | acetyl | acetyl | acetyl |
| 6808 | H | acetyl | acetyl | H | acetyl | acetyl | H | acetyl | H | H | H | H |
| 6809 | H | acetyl | acetyl | H | acetyl | acetyl | H | acetyl | H | H | H | acetyl |
| 6810 | H | acetyl | acetyl | H | acetyl | acetyl | H | acetyl | H | H | acetyl | H |
| 6811 | H | acetyl | acetyl | H | acetyl | acetyl | H | acetyl | H | H | acetyl | acetyl |
| 6812 | H | acetyl | acetyl | H | acetyl | acetyl | H | acetyl | H | acetyl | H | H |
| 6813 | H | acetyl | acetyl | H | acetyl | acetyl | H | acetyl | H | acetyl | H | acetyl |
| 6814 | H | acetyl | acetyl | H | acetyl | acetyl | H | acetyl | H | acetyl | acetyl | H |
| 6815 | H | acetyl | acetyl | H | acetyl | acetyl | H | acetyl | H | acetyl | acetyl | acetyl |
| 6816 | H | acetyl | acetyl | H | acetyl | acetyl | H | acetyl | acetyl | H | H | H |
| 6817 | H | acetyl | acetyl | H | acetyl | acetyl | H | acetyl | acetyl | H | H | acetyl |
| 6818 | H | acetyl | acetyl | H | acetyl | acetyl | H | acetyl | acetyl | H | acetyl | H |
| 6819 | H | acetyl | acetyl | H | acetyl | acetyl | H | acetyl | acetyl | H | acetyl | acetyl |
| 6820 | H | acetyl | acetyl | H | acetyl | acetyl | H | acetyl | acetyl | acetyl | H | H |
| 6821 | H | acetyl | acetyl | H | acetyl | acetyl | H | acetyl | acetyl | acetyl | H | acetyl |
| 6822 | H | acetyl | acetyl | H | acetyl | acetyl | H | acetyl | acetyl | acetyl | acetyl | H |
| 6823 | H | acetyl | acetyl | H | acetyl | acetyl | H | acetyl | acetyl | acetyl | acetyl | acetyl |
| 6824 | H | acetyl | acetyl | H | acetyl | acetyl | acetyl | H | H | H | H | H |
| 6825 | H | acetyl | acetyl | H | acetyl | acetyl | acetyl | H | H | H | H | acetyl |
| 6826 | H | acetyl | acetyl | H | acetyl | acetyl | acetyl | H | H | H | acetyl | H |
| 6827 | H | acetyl | acetyl | H | acetyl | acetyl | acetyl | H | H | H | acetyl | acetyl |
| 6828 | H | acetyl | acetyl | H | acetyl | acetyl | acetyl | H | H | acetyl | H | H |
| 6829 | H | acetyl | acetyl | H | acetyl | acetyl | acetyl | H | H | acetyl | H | acetyl |
| 6830 | H | acetyl | acetyl | H | acetyl | acetyl | acetyl | H | H | acetyl | acetyl | H |
| 6831 | H | acetyl | acetyl | H | acetyl | acetyl | acetyl | H | H | acetyl | acetyl | acetyl |
| 6832 | H | acetyl | acetyl | H | acetyl | acetyl | acetyl | H | acetyl | H | H | H |
| 6833 | H | acetyl | acetyl | H | acetyl | acetyl | acetyl | H | acetyl | H | H | acetyl |
| 6834 | H | acetyl | acetyl | H | acetyl | acetyl | acetyl | H | acetyl | H | acetyl | H |
| 6835 | H | acetyl | acetyl | H | acetyl | acetyl | acetyl | H | acetyl | H | acetyl | acetyl |
| 6836 | H | acetyl | acetyl | H | acetyl | acetyl | acetyl | H | acetyl | acetyl | H | H |
| 6837 | H | acetyl | acetyl | H | acetyl | acetyl | acetyl | H | acetyl | acetyl | H | acetyl |
| 6838 | H | acetyl | acetyl | H | acetyl | acetyl | acetyl | H | acetyl | acetyl | acetyl | H |
| 6839 | H | acetyl | acetyl | H | acetyl | acetyl | acetyl | H | acetyl | acetyl | acetyl | acetyl |
| 6840 | H | acetyl | acetyl | H | acetyl | acetyl | acetyl | acetyl | H | H | H | H |
| 6841 | H | acetyl | acetyl | H | acetyl | acetyl | acetyl | acetyl | H | H | H | acetyl |
| 6842 | H | acetyl | acetyl | H | acetyl | acetyl | acetyl | acetyl | H | H | acetyl | H |
| 6843 | H | acetyl | acetyl | H | acetyl | acetyl | acetyl | acetyl | H | H | acetyl | acetyl |
| 6844 | H | acetyl | acetyl | H | acetyl | acetyl | acetyl | acetyl | H | acetyl | H | H |
| 6845 | H | acetyl | acetyl | H | acetyl | acetyl | acetyl | acetyl | H | acetyl | H | acetyl |
| 6846 | H | acetyl | acetyl | H | acetyl | acetyl | acetyl | acetyl | H | acetyl | acetyl | H |
| 6847 | H | acetyl | acetyl | H | acetyl | acetyl | acetyl | acetyl | H | acetyl | acetyl | acetyl |
| 6848 | H | acetyl | acetyl | H | acetyl | acetyl | acetyl | acetyl | acetyl | H | H | H |
| 6849 | H | acetyl | acetyl | H | acetyl | acetyl | acetyl | acetyl | acetyl | H | H | acetyl |
| 6850 | H | acetyl | acetyl | H | acetyl | acetyl | acetyl | acetyl | acetyl | H | acetyl | H |
| 6851 | H | acetyl | acetyl | H | acetyl | acetyl | acetyl | acetyl | acetyl | H | acetyl | acetyl |
| 6852 | H | acetyl | acetyl | H | acetyl | acetyl | acetyl | acetyl | acetyl | acetyl | H | H |
| 6853 | H | acetyl | acetyl | H | acetyl | acetyl | acetyl | acetyl | acetyl | acetyl | H | acetyl |
| 6854 | H | acetyl | acetyl | H | acetyl | acetyl | acetyl | acetyl | acetyl | acetyl | acetyl | H |
| 6855 | H | acetyl | acetyl | H | acetyl | acetyl | acetyl | acetyl | acetyl | acetyl | acetyl | acetyl |
| 6856 | H | acetyl | acetyl | acetyl | H | H | H | H | H | H | H | H |
| 6857 | H | acetyl | acetyl | acetyl | H | H | H | H | H | H | H | acetyl |
| 6858 | H | acetyl | acetyl | acetyl | H | H | H | H | H | H | acetyl | H |
| 6859 | H | acetyl | acetyl | acetyl | H | H | H | H | H | H | acetyl | acetyl |
| 6860 | H | acetyl | acetyl | acetyl | H | H | H | H | H | acetyl | H | H |
| 6861 | H | acetyl | acetyl | acetyl | H | H | H | H | H | acetyl | H | acetyl |
| 6862 | H | acetyl | acetyl | acetyl | H | H | H | H | H | acetyl | acetyl | H |
| 6863 | H | acetyl | acetyl | acetyl | H | H | H | H | H | acetyl | acetyl | acetyl |
| 6864 | H | acetyl | acetyl | acetyl | H | H | H | H | acetyl | H | H | H |
| 6865 | H | acetyl | acetyl | acetyl | H | H | H | H | acetyl | H | H | acetyl |
| 6866 | H | acetyl | acetyl | acetyl | H | H | H | H | acetyl | H | acetyl | H |
| 6867 | H | acetyl | acetyl | acetyl | H | H | H | H | acetyl | H | acetyl | acetyl |
| 6868 | H | acetyl | acetyl | acetyl | H | H | H | H | acetyl | acetyl | H | H |
| 6869 | H | acetyl | acetyl | acetyl | H | H | H | H | acetyl | acetyl | H | acetyl |
| 6870 | H | acetyl | acetyl | acetyl | H | H | H | H | acetyl | acetyl | acetyl | H |
| 6871 | H | acetyl | acetyl | acetyl | H | H | H | H | acetyl | acetyl | acetyl | acetyl |
| 6872 | H | acetyl | acetyl | acetyl | H | H | H | acetyl | H | H | H | H |
| 6873 | H | acetyl | acetyl | acetyl | H | H | H | acetyl | H | H | H | acetyl |
| 6874 | H | acetyl | acetyl | acetyl | H | H | H | acetyl | H | H | acetyl | H |
| 6875 | H | acetyl | acetyl | acetyl | H | H | H | acetyl | H | H | acetyl | acetyl |
| 6876 | H | acetyl | acetyl | acetyl | H | H | H | acetyl | H | acetyl | H | H |
| 6877 | H | acetyl | acetyl | acetyl | H | H | H | acetyl | H | acetyl | H | acetyl |
| 6878 | H | acetyl | acetyl | acetyl | H | H | H | acetyl | H | acetyl | acetyl | H |
| 6879 | H | acetyl | acetyl | acetyl | H | H | H | acetyl | H | acetyl | acetyl | acetyl |
| 6880 | H | acetyl | acetyl | acetyl | H | H | H | acetyl | acetyl | H | H | H |
| 6881 | H | acetyl | acetyl | acetyl | H | H | H | acetyl | acetyl | H | H | acetyl |
| 6882 | H | acetyl | acetyl | acetyl | H | H | H | acetyl | acetyl | H | acetyl | H |
| 6883 | H | acetyl | acetyl | acetyl | H | H | H | acetyl | acetyl | H | acetyl | acetyl |
| 6884 | H | acetyl | acetyl | acetyl | H | H | H | acetyl | acetyl | acetyl | H | H |
| 6885 | H | acetyl | acetyl | acetyl | H | H | H | acetyl | acetyl | acetyl | H | acetyl |
| 6886 | H | acetyl | acetyl | acetyl | H | H | H | acetyl | acetyl | acetyl | acetyl | H |
| 6887 | H | acetyl | acetyl | acetyl | H | H | H | acetyl | acetyl | acetyl | acetyl | acetyl |
| 6888 | H | acetyl | acetyl | acetyl | H | H | acetyl | H | H | H | H | H |
| 6889 | H | acetyl | acetyl | acetyl | H | H | acetyl | H | H | H | H | acetyl |
| 6890 | H | acetyl | acetyl | acetyl | H | H | acetyl | H | H | H | acetyl | H |
| 6891 | H | acetyl | acetyl | acetyl | H | H | acetyl | H | H | H | acetyl | acetyl |
| 6892 | H | acetyl | acetyl | acetyl | H | H | acetyl | H | H | acetyl | H | H |
| 6893 | H | acetyl | acetyl | acetyl | H | H | acetyl | H | H | acetyl | H | acetyl |
| 6894 | H | acetyl | acetyl | acetyl | H | H | acetyl | H | H | acetyl | acetyl | H |
| 6895 | H | acetyl | acetyl | acetyl | H | H | acetyl | H | H | acetyl | acetyl | acetyl |
| 6896 | H | acetyl | acetyl | acetyl | H | H | acetyl | H | acetyl | H | H | H |
| 6897 | H | acetyl | acetyl | acetyl | H | H | acetyl | H | acetyl | H | H | acetyl |
| 6898 | H | acetyl | acetyl | acetyl | H | H | acetyl | H | acetyl | H | acetyl | H |
| 6899 | H | acetyl | acetyl | acetyl | H | H | acetyl | H | acetyl | H | acetyl | acetyl |
| 6900 | H | acetyl | acetyl | acetyl | H | H | acetyl | H | acetyl | acetyl | H | H |
| 6901 | H | acetyl | acetyl | acetyl | H | H | acetyl | H | acetyl | acetyl | H | acetyl |
| 6902 | H | acetyl | acetyl | acetyl | H | H | acetyl | H | acetyl | acetyl | acetyl | H |
| 6903 | H | acetyl | acetyl | acetyl | H | H | acetyl | H | acetyl | acetyl | acetyl | acetyl |
| 6904 | H | acetyl | acetyl | acetyl | H | H | acetyl | acetyl | H | H | H | H |
| 6905 | H | acetyl | acetyl | acetyl | H | H | acetyl | acetyl | H | H | H | acetyl |
| 6906 | H | acetyl | acetyl | acetyl | H | H | acetyl | acetyl | H | H | acetyl | H |
| 6907 | H | acetyl | acetyl | acetyl | H | H | acetyl | acetyl | H | H | acetyl | acetyl |
| 6908 | H | acetyl | acetyl | acetyl | H | H | acetyl | acetyl | H | acetyl | H | H |
| 6909 | H | acetyl | acetyl | acetyl | H | H | acetyl | acetyl | H | acetyl | H | acetyl |
| 6910 | H | acetyl | acetyl | acetyl | H | H | acetyl | acetyl | H | acetyl | acetyl | H |
| 6911 | H | acetyl | acetyl | acetyl | H | H | acetyl | acetyl | H | acetyl | acetyl | acetyl |
| 6912 | H | acetyl | acetyl | acetyl | H | H | acetyl | acetyl | acetyl | H | H | H |
| 6913 | H | acetyl | acetyl | acetyl | H | H | acetyl | acetyl | acetyl | H | H | acetyl |
| 6914 | H | acetyl | acetyl | acetyl | H | H | acetyl | acetyl | acetyl | H | acetyl | H |
| 6915 | H | acetyl | acetyl | acetyl | H | H | acetyl | acetyl | acetyl | H | acetyl | acetyl |
| 6916 | H | acetyl | acetyl | acetyl | H | H | acetyl | acetyl | acetyl | acetyl | H | H |
| 6917 | H | acetyl | acetyl | acetyl | H | H | acetyl | acetyl | acetyl | acetyl | H | acetyl |
| 6918 | H | acetyl | acetyl | acetyl | H | H | acetyl | acetyl | acetyl | acetyl | acetyl | H |
| 6919 | H | acetyl | acetyl | acetyl | H | H | acetyl | acetyl | acetyl | acetyl | acetyl | acetyl |
| 6920 | H | acetyl | acetyl | acetyl | H | acetyl | H | H | H | H | H | H |
| 6921 | H | acetyl | acetyl | acetyl | H | acetyl | H | H | H | H | H | acetyl |
| 6922 | H | acetyl | acetyl | acetyl | H | acetyl | H | H | H | H | acetyl | H |
| 6923 | H | acetyl | acetyl | acetyl | H | acetyl | H | H | H | H | acetyl | acetyl |
| 6924 | H | acetyl | acetyl | acetyl | H | acetyl | H | H | H | acetyl | H | H |
| 6925 | H | acetyl | acetyl | acetyl | H | acetyl | H | H | H | acetyl | H | acetyl |
| 6926 | H | acetyl | acetyl | acetyl | H | acetyl | H | H | H | acetyl | acetyl | H |
| 6927 | H | acetyl | acetyl | acetyl | H | acetyl | H | H | H | acetyl | acetyl | acetyl |
| 6928 | H | acetyl | acetyl | acetyl | H | acetyl | H | H | acetyl | H | H | H |
| 6929 | H | acetyl | acetyl | acetyl | H | acetyl | H | H | acetyl | H | H | acetyl |
| 6930 | H | acetyl | acetyl | acetyl | H | acetyl | H | H | acetyl | H | acetyl | H |
| 6931 | H | acetyl | acetyl | acetyl | H | acetyl | H | H | acetyl | H | acetyl | acetyl |
| 6932 | H | acetyl | acetyl | acetyl | H | acetyl | H | H | acetyl | acetyl | H | H |
| 6933 | H | acetyl | acetyl | acetyl | H | acetyl | H | H | acetyl | acetyl | H | acetyl |
| 6934 | H | acetyl | acetyl | acetyl | H | acetyl | H | H | acetyl | acetyl | acetyl | H |
| 6935 | H | acetyl | acetyl | acetyl | H | acetyl | H | H | acetyl | acetyl | acetyl | acetyl |
| 6936 | H | acetyl | acetyl | acetyl | H | acetyl | H | acetyl | H | H | H | H |
| 6937 | H | acetyl | acetyl | acetyl | H | acetyl | H | acetyl | H | H | H | acetyl |
| 6938 | H | acetyl | acetyl | acetyl | H | acetyl | H | acetyl | H | H | acetyl | H |
| 6939 | H | acetyl | acetyl | acetyl | H | acetyl | H | acetyl | H | H | acetyl | acetyl |
| 6940 | H | acetyl | acetyl | acetyl | H | acetyl | H | acetyl | H | acetyl | H | H |
| 6941 | H | acetyl | acetyl | acetyl | H | acetyl | H | acetyl | H | acetyl | H | acetyl |
| 6942 | H | acetyl | acetyl | acetyl | H | acetyl | H | acetyl | H | acetyl | acetyl | H |
| 6943 | H | acetyl | acetyl | acetyl | H | acetyl | H | acetyl | H | acetyl | acetyl | acetyl |
| 6944 | H | acetyl | acetyl | acetyl | H | acetyl | H | acetyl | acetyl | H | H | H |
| 6945 | H | acetyl | acetyl | acetyl | H | acetyl | H | acetyl | acetyl | H | H | acetyl |
| 6946 | H | acetyl | acetyl | acetyl | H | acetyl | H | acetyl | acetyl | H | acetyl | H |
| 6947 | H | acetyl | acetyl | acetyl | H | acetyl | H | acetyl | acetyl | H | acetyl | acetyl |
| 6948 | H | acetyl | acetyl | acetyl | H | acetyl | H | acetyl | acetyl | acetyl | H | H |
| 6949 | H | acetyl | acetyl | acetyl | H | acetyl | H | acetyl | acetyl | acetyl | H | acetyl |
| 6950 | H | acetyl | acetyl | acetyl | H | acetyl | H | acetyl | acetyl | acetyl | acetyl | H |
| 6951 | H | acetyl | acetyl | acetyl | H | acetyl | H | acetyl | acetyl | acetyl | acetyl | acetyl |
| 6952 | H | acetyl | acetyl | acetyl | H | acetyl | acetyl | H | H | H | H | H |
| 6953 | H | acetyl | acetyl | acetyl | H | acetyl | acetyl | H | H | H | H | acetyl |
| 6954 | H | acetyl | acetyl | acetyl | H | acetyl | acetyl | H | H | H | acetyl | H |
| 6955 | H | acetyl | acetyl | acetyl | H | acetyl | acetyl | H | H | H | acetyl | acetyl |
| 6956 | H | acetyl | acetyl | acetyl | H | acetyl | acetyl | H | H | acetyl | H | H |
| 6957 | H | acetyl | acetyl | acetyl | H | acetyl | acetyl | H | H | acetyl | H | acetyl |
| 6958 | H | acetyl | acetyl | acetyl | H | acetyl | acetyl | H | H | acetyl | acetyl | H |
| 6959 | H | acetyl | acetyl | acetyl | H | acetyl | acetyl | H | H | acetyl | acetyl | acetyl |
| 6960 | H | acetyl | acetyl | acetyl | H | acetyl | acetyl | H | acetyl | H | H | H |
| 6961 | H | acetyl | acetyl | acetyl | H | acetyl | acetyl | H | acetyl | H | H | acetyl |
| 6962 | H | acetyl | acetyl | acetyl | H | acetyl | acetyl | H | acetyl | H | acetyl | H |
| 6963 | H | acetyl | acetyl | acetyl | H | acetyl | acetyl | H | acetyl | H | acetyl | acetyl |
| 6964 | H | acetyl | acetyl | acetyl | H | acetyl | acetyl | H | acetyl | acetyl | H | H |
| 6965 | H | acetyl | acetyl | acetyl | H | acetyl | acetyl | H | acetyl | acetyl | H | acetyl |
| 6966 | H | acetyl | acetyl | acetyl | H | acetyl | acetyl | H | acetyl | acetyl | acetyl | H |
| 6967 | H | acetyl | acetyl | acetyl | H | acetyl | acetyl | H | acetyl | acetyl | acetyl | acetyl |
| 6968 | H | acetyl | acetyl | acetyl | H | acetyl | acetyl | acetyl | H | H | H | H |
| 6969 | H | acetyl | acetyl | acetyl | H | acetyl | acetyl | acetyl | H | H | H | acetyl |
| 6970 | H | acetyl | acetyl | acetyl | H | acetyl | acetyl | acetyl | H | H | acetyl | H |
| 6971 | H | acetyl | acetyl | acetyl | H | acetyl | acetyl | acetyl | H | H | acetyl | acetyl |
| 6972 | H | acetyl | acetyl | acetyl | H | acetyl | acetyl | acetyl | H | acetyl | H | H |
| 6973 | H | acetyl | acetyl | acetyl | H | acetyl | acetyl | acetyl | H | acetyl | H | acetyl |
| 6974 | H | acetyl | acetyl | acetyl | H | acetyl | acetyl | acetyl | H | acetyl | acetyl | H |
| 6975 | H | acetyl | acetyl | acetyl | H | acetyl | acetyl | acetyl | H | acetyl | acetyl | acetyl |
| 6976 | H | acetyl | acetyl | acetyl | H | acetyl | acetyl | acetyl | acetyl | H | H | H |
| 6977 | H | acetyl | acetyl | acetyl | H | acetyl | acetyl | acetyl | acetyl | H | H | acetyl |
| 6978 | H | acetyl | acetyl | acetyl | H | acetyl | acetyl | acetyl | acetyl | H | acetyl | H |
| 6979 | H | acetyl | acetyl | acetyl | H | acetyl | acetyl | acetyl | acetyl | H | acetyl | acetyl |
| 6980 | H | acetyl | acetyl | acetyl | H | acetyl | acetyl | acetyl | acetyl | acetyl | H | H |
| 6981 | H | acetyl | acetyl | acetyl | H | acetyl | acetyl | acetyl | acetyl | acetyl | H | acetyl |
| 6982 | H | acetyl | acetyl | acetyl | H | acetyl | acetyl | acetyl | acetyl | acetyl | acetyl | H |
| 6983 | H | acetyl | acetyl | acetyl | H | acetyl | acetyl | acetyl | acetyl | acetyl | acetyl | acetyl |
| 6984 | H | acetyl | acetyl | acetyl | acetyl | H | H | H | H | H | H | H |
| 6985 | H | acetyl | acetyl | acetyl | acetyl | H | H | H | H | H | H | acetyl |
| 6986 | H | acetyl | acetyl | acetyl | acetyl | H | H | H | H | H | acetyl | H |
| 6987 | H | acetyl | acetyl | acetyl | acetyl | H | H | H | H | H | acetyl | acetyl |
| 6988 | H | acetyl | acetyl | acetyl | acetyl | H | H | H | H | acetyl | H | H |
| 6989 | H | acetyl | acetyl | acetyl | acetyl | H | H | H | H | acetyl | H | acetyl |
| 6990 | H | acetyl | acetyl | acetyl | acetyl | H | H | H | H | acetyl | acetyl | H |
| 6991 | H | acetyl | acetyl | acetyl | acetyl | H | H | H | H | acetyl | acetyl | acetyl |
| 6992 | H | acetyl | acetyl | acetyl | acetyl | H | H | H | acetyl | H | H | H |
| 6993 | H | acetyl | acetyl | acetyl | acetyl | H | H | H | acetyl | H | H | acetyl |
| 6994 | H | acetyl | acetyl | acetyl | acetyl | H | H | H | acetyl | H | acetyl | H |
| 6995 | H | acetyl | acetyl | acetyl | acetyl | H | H | H | acetyl | H | acetyl | acetyl |
| 6996 | H | acetyl | acetyl | acetyl | acetyl | H | H | H | acetyl | acetyl | H | H |
| 6997 | H | acetyl | acetyl | acetyl | acetyl | H | H | H | acetyl | acetyl | H | acetyl |
| 6998 | H | acetyl | acetyl | acetyl | acetyl | H | H | H | acetyl | acetyl | acetyl | H |
| 6999 | H | acetyl | acetyl | acetyl | acetyl | H | H | H | acetyl | acetyl | acetyl | acetyl |
| 7000 | H | acetyl | acetyl | acetyl | acetyl | H | H | acetyl | H | H | H | H |
| 7001 | H | acetyl | acetyl | acetyl | acetyl | H | H | acetyl | H | H | H | acetyl |
| 7002 | H | acetyl | acetyl | acetyl | acetyl | H | H | acetyl | H | H | acetyl | H |
| 7003 | H | acetyl | acetyl | acetyl | acetyl | H | H | acetyl | H | H | acetyl | acetyl |
| 7004 | H | acetyl | acetyl | acetyl | acetyl | H | H | acetyl | H | acetyl | H | H |
| 7005 | H | acetyl | acetyl | acetyl | acetyl | H | H | acetyl | H | acetyl | H | acetyl |
| 7006 | H | acetyl | acetyl | acetyl | acetyl | H | H | acetyl | H | acetyl | acetyl | H |
| 7007 | H | acetyl | acetyl | acetyl | acetyl | H | H | acetyl | H | acetyl | acetyl | acetyl |
| 7008 | H | acetyl | acetyl | acetyl | acetyl | H | H | acetyl | acetyl | H | H | H |
| 7009 | H | acetyl | acetyl | acetyl | acetyl | H | H | acetyl | acetyl | H | H | acetyl |
| 7010 | H | acetyl | acetyl | acetyl | acetyl | H | H | acetyl | acetyl | H | acetyl | H |
| 7011 | H | acetyl | acetyl | acetyl | acetyl | H | H | acetyl | acetyl | H | acetyl | acetyl |
| 7012 | H | acetyl | acetyl | acetyl | acetyl | H | H | acetyl | acetyl | acetyl | H | H |
| 7013 | H | acetyl | acetyl | acetyl | acetyl | H | H | acetyl | acetyl | acetyl | H | acetyl |
| 7014 | H | acetyl | acetyl | acetyl | acetyl | H | H | acetyl | acetyl | acetyl | acetyl | H |
| 7015 | H | acetyl | acetyl | acetyl | acetyl | H | H | acetyl | acetyl | acetyl | acetyl | acetyl |
| 7016 | H | acetyl | acetyl | acetyl | acetyl | H | acetyl | H | H | H | H | H |
| 7017 | H | acetyl | acetyl | acetyl | acetyl | H | acetyl | H | H | H | H | acetyl |
| 7018 | H | acetyl | acetyl | acetyl | acetyl | H | acetyl | H | H | H | acetyl | H |
| 7019 | H | acetyl | acetyl | acetyl | acetyl | H | acetyl | H | H | H | acetyl | acetyl |
| 7020 | H | acetyl | acetyl | acetyl | acetyl | H | acetyl | H | H | acetyl | H | H |
| 7021 | H | acetyl | acetyl | acetyl | acetyl | H | acetyl | H | H | acetyl | H | acetyl |
| 7022 | H | acetyl | acetyl | acetyl | acetyl | H | acetyl | H | H | acetyl | acetyl | H |
| 7023 | H | acetyl | acetyl | acetyl | acetyl | H | acetyl | H | H | acetyl | acetyl | acetyl |
| 7024 | H | acetyl | acetyl | acetyl | acetyl | H | acetyl | H | acetyl | H | H | H |
| 7025 | H | acetyl | acetyl | acetyl | acetyl | H | acetyl | H | acetyl | H | H | acetyl |
| 7026 | H | acetyl | acetyl | acetyl | acetyl | H | acetyl | H | acetyl | H | acetyl | H |
| 7027 | H | acetyl | acetyl | acetyl | acetyl | H | acetyl | H | acetyl | H | acetyl | acetyl |
| 7028 | H | acetyl | acetyl | acetyl | acetyl | H | acetyl | H | acetyl | acetyl | H | H |
| 7029 | H | acetyl | acetyl | acetyl | acetyl | H | acetyl | H | acetyl | acetyl | H | acetyl |
| 7030 | H | acetyl | acetyl | acetyl | acetyl | H | acetyl | H | acetyl | acetyl | acetyl | H |
| 7031 | H | acetyl | acetyl | acetyl | acetyl | H | acetyl | H | acetyl | acetyl | acetyl | acetyl |
| 7032 | H | acetyl | acetyl | acetyl | acetyl | H | acetyl | acetyl | H | H | H | H |
| 7033 | H | acetyl | acetyl | acetyl | acetyl | H | acetyl | acetyl | H | H | H | acetyl |
| 7034 | H | acetyl | acetyl | acetyl | acetyl | H | acetyl | acetyl | H | H | acetyl | H |
| 7035 | H | acetyl | acetyl | acetyl | acetyl | H | acetyl | acetyl | H | H | acetyl | acetyl |
| 7036 | H | acetyl | acetyl | acetyl | acetyl | H | acetyl | acetyl | H | acetyl | H | H |
| 7037 | H | acetyl | acetyl | acetyl | acetyl | H | acetyl | acetyl | H | acetyl | H | acetyl |
| 7038 | H | acetyl | acetyl | acetyl | acetyl | H | acetyl | acetyl | H | acetyl | acetyl | H |
| 7039 | H | acetyl | acetyl | acetyl | acetyl | H | acetyl | acetyl | H | acetyl | acetyl | acetyl |
| 7040 | H | acetyl | acetyl | acetyl | acetyl | H | acetyl | acetyl | acetyl | H | H | H |
| 7041 | H | acetyl | acetyl | acetyl | acetyl | H | acetyl | acetyl | acetyl | H | H | acetyl |
| 7042 | H | acetyl | acetyl | acetyl | acetyl | H | acetyl | acetyl | acetyl | H | acetyl | H |
| 7043 | H | acetyl | acetyl | acetyl | acetyl | H | acetyl | acetyl | acetyl | H | acetyl | acetyl |
| 7044 | H | acetyl | acetyl | acetyl | acetyl | H | acetyl | acetyl | acetyl | acetyl | H | H |
| 7045 | H | acetyl | acetyl | acetyl | acetyl | H | acetyl | acetyl | acetyl | acetyl | H | acetyl |
| 7046 | H | acetyl | acetyl | acetyl | acetyl | H | acetyl | acetyl | acetyl | acetyl | acetyl | H |
| 7047 | H | acetyl | acetyl | acetyl | acetyl | H | acetyl | acetyl | acetyl | acetyl | acetyl | acetyl |
| 7048 | H | acetyl | acetyl | acetyl | acetyl | acetyl | H | H | H | H | H | H |
| 7049 | H | acetyl | acetyl | acetyl | acetyl | acetyl | H | H | H | H | H | acetyl |
| 7050 | H | acetyl | acetyl | acetyl | acetyl | acetyl | H | H | H | H | acetyl | H |
| 7051 | H | acetyl | acetyl | acetyl | acetyl | acetyl | H | H | H | H | acetyl | acetyl |
| 7052 | H | acetyl | acetyl | acetyl | acetyl | acetyl | H | H | H | acetyl | H | H |
| 7053 | H | acetyl | acetyl | acetyl | acetyl | acetyl | H | H | H | acetyl | H | acetyl |
| 7054 | H | acetyl | acetyl | acetyl | acetyl | acetyl | H | H | H | acetyl | acetyl | H |
| 7055 | H | acetyl | acetyl | acetyl | acetyl | acetyl | H | H | H | acetyl | acetyl | acetyl |
| 7056 | H | acetyl | acetyl | acetyl | acetyl | acetyl | H | H | acetyl | H | H | H |
| 7057 | H | acetyl | acetyl | acetyl | acetyl | acetyl | H | H | acetyl | H | H | acetyl |
| 7058 | H | acetyl | acetyl | acetyl | acetyl | acetyl | H | H | acetyl | H | acetyl | H |
| 7059 | H | acetyl | acetyl | acetyl | acetyl | acetyl | H | H | acetyl | H | acetyl | acetyl |
| 7060 | H | acetyl | acetyl | acetyl | acetyl | acetyl | H | H | acetyl | acetyl | H | H |
| 7061 | H | acetyl | acetyl | acetyl | acetyl | acetyl | H | H | acetyl | acetyl | H | acetyl |
| 7062 | H | acetyl | acetyl | acetyl | acetyl | acetyl | H | H | acetyl | acetyl | acetyl | H |
| 7063 | H | acetyl | acetyl | acetyl | acetyl | acetyl | H | H | acetyl | acetyl | acetyl | acetyl |
| 7064 | H | acetyl | acetyl | acetyl | acetyl | acetyl | H | acetyl | H | H | H | H |
| 7065 | H | acetyl | acetyl | acetyl | acetyl | acetyl | H | acetyl | H | H | H | acetyl |
| 7066 | H | acetyl | acetyl | acetyl | acetyl | acetyl | H | acetyl | H | H | acetyl | H |
| 7067 | H | acetyl | acetyl | acetyl | acetyl | acetyl | H | acetyl | H | H | acetyl | acetyl |
| 7068 | H | acetyl | acetyl | acetyl | acetyl | acetyl | H | acetyl | H | acetyl | H | H |
| 7069 | H | acetyl | acetyl | acetyl | acetyl | acetyl | H | acetyl | H | acetyl | H | acetyl |
| 7070 | H | acetyl | acetyl | acetyl | acetyl | acetyl | H | acetyl | H | acetyl | acetyl | H |
| 7071 | H | acetyl | acetyl | acetyl | acetyl | acetyl | H | acetyl | H | acetyl | acetyl | acetyl |
| 7072 | H | acetyl | acetyl | acetyl | acetyl | acetyl | H | acetyl | acetyl | H | H | H |
| 7073 | H | acetyl | acetyl | acetyl | acetyl | acetyl | H | acetyl | acetyl | H | H | acetyl |
| 7074 | H | acetyl | acetyl | acetyl | acetyl | acetyl | H | acetyl | acetyl | H | acetyl | H |
| 7075 | H | acetyl | acetyl | acetyl | acetyl | acetyl | H | acetyl | acetyl | H | acetyl | acetyl |
| 7076 | H | acetyl | acetyl | acetyl | acetyl | acetyl | H | acetyl | acetyl | acetyl | H | H |
| 7077 | H | acetyl | acetyl | acetyl | acetyl | acetyl | H | acetyl | acetyl | acetyl | H | acetyl |
| 7078 | H | acetyl | acetyl | acetyl | acetyl | acetyl | H | acetyl | acetyl | acetyl | acetyl | H |
| 7079 | H | acetyl | acetyl | acetyl | acetyl | acetyl | H | acetyl | acetyl | acetyl | acetyl | acetyl |
| 7080 | H | acetyl | acetyl | acetyl | acetyl | acetyl | acetyl | H | H | H | H | H |
| 7081 | H | acetyl | acetyl | acetyl | acetyl | acetyl | acetyl | H | H | H | H | acetyl |
| 7082 | H | acetyl | acetyl | acetyl | acetyl | acetyl | acetyl | H | H | H | acetyl | H |
| 7083 | H | acetyl | acetyl | acetyl | acetyl | acetyl | acetyl | H | H | H | acetyl | acetyl |
| 7084 | H | acetyl | acetyl | acetyl | acetyl | acetyl | acetyl | H | H | acetyl | H | H |
| 7085 | H | acetyl | acetyl | acetyl | acetyl | acetyl | acetyl | H | H | acetyl | H | acetyl |
| 7086 | H | acetyl | acetyl | acetyl | acetyl | acetyl | acetyl | H | H | acetyl | acetyl | H |
| 7087 | H | acetyl | acetyl | acetyl | acetyl | acetyl | acetyl | H | H | acetyl | acetyl | acetyl |
| 7088 | H | acetyl | acetyl | acetyl | acetyl | acetyl | acetyl | H | acetyl | H | H | H |
| 7089 | H | acetyl | acetyl | acetyl | acetyl | acetyl | acetyl | H | acetyl | H | H | acetyl |
| 7090 | H | acetyl | acetyl | acetyl | acetyl | acetyl | acetyl | H | acetyl | H | acetyl | H |
| 7091 | H | acetyl | acetyl | acetyl | acetyl | acetyl | acetyl | H | acetyl | H | acetyl | acetyl |
| 7092 | H | acetyl | acetyl | acetyl | acetyl | acetyl | acetyl | H | acetyl | acetyl | H | H |
| 7093 | H | acetyl | acetyl | acetyl | acetyl | acetyl | acetyl | H | acetyl | acetyl | H | acetyl |
| 7094 | H | acetyl | acetyl | acetyl | acetyl | acetyl | acetyl | H | acetyl | acetyl | acetyl | H |
| 7095 | H | acetyl | acetyl | acetyl | acetyl | acetyl | acetyl | H | acetyl | acetyl | acetyl | acetyl |
| 7096 | H | acetyl | acetyl | acetyl | acetyl | acetyl | acetyl | acetyl | H | H | H | H |
| 7097 | H | acetyl | acetyl | acetyl | acetyl | acetyl | acetyl | acetyl | H | H | H | acetyl |
| 7098 | H | acetyl | acetyl | acetyl | acetyl | acetyl | acetyl | acetyl | H | H | acetyl | H |
| 7099 | H | acetyl | acetyl | acetyl | acetyl | acetyl | acetyl | acetyl | H | H | acetyl | acetyl |
| 7100 | H | acetyl | acetyl | acetyl | acetyl | acetyl | acetyl | acetyl | H | acetyl | H | H |
| 7101 | H | acetyl | acetyl | acetyl | acetyl | acetyl | acetyl | acetyl | H | acetyl | H | acetyl |
| 7102 | H | acetyl | acetyl | acetyl | acetyl | acetyl | acetyl | acetyl | H | acetyl | acetyl | H |
| 7103 | H | acetyl | acetyl | acetyl | acetyl | acetyl | acetyl | acetyl | H | acetyl | acetyl | acetyl |
| 7104 | H | acetyl | acetyl | acetyl | acetyl | acetyl | acetyl | acetyl | acetyl | H | H | H |
| 7105 | H | acetyl | acetyl | acetyl | acetyl | acetyl | acetyl | acetyl | acetyl | H | H | acetyl |
| 7106 | H | acetyl | acetyl | acetyl | acetyl | acetyl | acetyl | acetyl | acetyl | H | acetyl | H |
| 7107 | H | acetyl | acetyl | acetyl | acetyl | acetyl | acetyl | acetyl | acetyl | H | acetyl | acetyl |
| 7108 | H | acetyl | acetyl | acetyl | acetyl | acetyl | acetyl | acetyl | acetyl | acetyl | H | H |
| 7109 | H | acetyl | acetyl | acetyl | acetyl | acetyl | acetyl | acetyl | acetyl | acetyl | H | acetyl |
| 7110 | H | acetyl | acetyl | acetyl | acetyl | acetyl | acetyl | acetyl | acetyl | acetyl | acetyl | H |
| 7111 | H | acetyl | acetyl | acetyl | acetyl | acetyl | acetyl | acetyl | acetyl | acetyl | acetyl | acetyl |
| 7112 | acetyl | H | H | H | H | H | H | H | H | H | H | H |
| 7113 | acetyl | H | H | H | H | H | H | H | H | H | H | acetyl |
| 7114 | acetyl | H | H | H | H | H | H | H | H | H | acetyl | H |
| 7115 | acetyl | H | H | H | H | H | H | H | H | H | acetyl | acetyl |
| 7116 | acetyl | H | H | H | H | H | H | H | H | acetyl | H | H |
| 7117 | acetyl | H | H | H | H | H | H | H | H | acetyl | H | acetyl |
| 7118 | acetyl | H | H | H | H | H | H | H | H | acetyl | acetyl | H |
| 7119 | acetyl | H | H | H | H | H | H | H | H | acetyl | acetyl | acetyl |
| 7120 | acetyl | H | H | H | H | H | H | H | acetyl | H | H | H |
| 7121 | acetyl | H | H | H | H | H | H | H | acetyl | H | H | acetyl |
| 7122 | acetyl | H | H | H | H | H | H | H | acetyl | H | acetyl | H |
| 7123 | acetyl | H | H | H | H | H | H | H | acetyl | H | acetyl | acetyl |
| 7124 | acetyl | H | H | H | H | H | H | H | acetyl | acetyl | H | H |
| 7125 | acetyl | H | H | H | H | H | H | H | acetyl | acetyl | H | acetyl |
| 7126 | acetyl | H | H | H | H | H | H | H | acetyl | acetyl | acetyl | H |
| 7127 | acetyl | H | H | H | H | H | H | H | acetyl | acetyl | acetyl | acetyl |
| 7128 | acetyl | H | H | H | H | H | H | acetyl | H | H | H | H |
| 7129 | acetyl | H | H | H | H | H | H | acetyl | H | H | H | acetyl |
| 7130 | acetyl | H | H | H | H | H | H | acetyl | H | H | acetyl | H |
| 7131 | acetyl | H | H | H | H | H | H | acetyl | H | H | acetyl | acetyl |
| 7132 | acetyl | H | H | H | H | H | H | acetyl | H | acetyl | H | H |
| 7133 | acetyl | H | H | H | H | H | H | acetyl | H | acetyl | H | acetyl |
| 7134 | acetyl | H | H | H | H | H | H | acetyl | H | acetyl | acetyl | H |
| 7135 | acetyl | H | H | H | H | H | H | acetyl | H | acetyl | acetyl | acetyl |
| 7136 | acetyl | H | H | H | H | H | H | acetyl | acetyl | H | H | H |
| 7137 | acetyl | H | H | H | H | H | H | acetyl | acetyl | H | H | acetyl |
| 7138 | acetyl | H | H | H | H | H | H | acetyl | acetyl | H | acetyl | H |
| 7139 | acetyl | H | H | H | H | H | H | acetyl | acetyl | H | acetyl | acetyl |
| 7140 | acetyl | H | H | H | H | H | H | acetyl | acetyl | acetyl | H | H |
| 7141 | acetyl | H | H | H | H | H | H | acetyl | acetyl | acetyl | H | acetyl |
| 7142 | acetyl | H | H | H | H | H | H | acetyl | acetyl | acetyl | acetyl | H |
| 7143 | acetyl | H | H | H | H | H | H | acetyl | acetyl | acetyl | acetyl | acetyl |
| 7144 | acetyl | H | H | H | H | H | acetyl | H | H | H | H | H |
| 7145 | acetyl | H | H | H | H | H | acetyl | H | H | H | H | acetyl |
| 7146 | acetyl | H | H | H | H | H | acetyl | H | H | H | acetyl | H |
| 7147 | acetyl | H | H | H | H | H | acetyl | H | H | H | acetyl | acetyl |
| 7148 | acetyl | H | H | H | H | H | acetyl | H | H | acetyl | H | H |
| 7149 | acetyl | H | H | H | H | H | acetyl | H | H | acetyl | H | acetyl |
| 7150 | acetyl | H | H | H | H | H | acetyl | H | H | acetyl | acetyl | H |
| 7151 | acetyl | H | H | H | H | H | acetyl | H | H | acetyl | acetyl | acetyl |
| 7152 | acetyl | H | H | H | H | H | acetyl | H | acetyl | H | H | H |
| 7153 | acetyl | H | H | H | H | H | acetyl | H | acetyl | H | H | acetyl |
| 7154 | acetyl | H | H | H | H | H | acetyl | H | acetyl | H | acetyl | H |
| 7155 | acetyl | H | H | H | H | H | acetyl | H | acetyl | H | acetyl | acetyl |
| 7156 | acetyl | H | H | H | H | H | acetyl | H | acetyl | acetyl | H | H |
| 7157 | acetyl | H | H | H | H | H | acetyl | H | acetyl | acetyl | H | acetyl |
| 7158 | acetyl | H | H | H | H | H | acetyl | H | acetyl | acetyl | acetyl | H |
| 7159 | acetyl | H | H | H | H | H | acetyl | H | acetyl | acetyl | acetyl | acetyl |
| 7160 | acetyl | H | H | H | H | H | acetyl | acetyl | H | H | H | H |
| 7161 | acetyl | H | H | H | H | H | acetyl | acetyl | H | H | H | acetyl |
| 7162 | acetyl | H | H | H | H | H | acetyl | acetyl | H | H | acetyl | H |
| 7163 | acetyl | H | H | H | H | H | acetyl | acetyl | H | H | acetyl | acetyl |
| 7164 | acetyl | H | H | H | H | H | acetyl | acetyl | H | acetyl | H | H |
| 7165 | acetyl | H | H | H | H | H | acetyl | acetyl | H | acetyl | H | acetyl |
| 7166 | acetyl | H | H | H | H | H | acetyl | acetyl | H | acetyl | acetyl | H |
| 7167 | acetyl | H | H | H | H | H | acetyl | acetyl | H | acetyl | acetyl | acetyl |
| 7168 | acetyl | H | H | H | H | H | acetyl | acetyl | acetyl | H | H | H |
| 7169 | acetyl | H | H | H | H | H | acetyl | acetyl | acetyl | H | H | acetyl |
| 7170 | acetyl | H | H | H | H | H | acetyl | acetyl | acetyl | H | acetyl | H |
| 7171 | acetyl | H | H | H | H | H | acetyl | acetyl | acetyl | H | acetyl | acetyl |
| 7172 | acetyl | H | H | H | H | H | acetyl | acetyl | acetyl | acetyl | H | H |
| 7173 | acetyl | H | H | H | H | H | acetyl | acetyl | acetyl | acetyl | H | acetyl |
| 7174 | acetyl | H | H | H | H | H | acetyl | acetyl | acetyl | acetyl | acetyl | H |
| 7175 | acetyl | H | H | H | H | H | acetyl | acetyl | acetyl | acetyl | acetyl | acetyl |
| 7176 | acetyl | H | H | H | H | acetyl | H | H | H | H | H | H |
| 7177 | acetyl | H | H | H | H | acetyl | H | H | H | H | H | acetyl |
| 7178 | acetyl | H | H | H | H | acetyl | H | H | H | H | acetyl | H |
| 7179 | acetyl | H | H | H | H | acetyl | H | H | H | H | acetyl | acetyl |
| 7180 | acetyl | H | H | H | H | acetyl | H | H | H | acetyl | H | H |
| 7181 | acetyl | H | H | H | H | acetyl | H | H | H | acetyl | H | acetyl |
| 7182 | acetyl | H | H | H | H | acetyl | H | H | H | acetyl | acetyl | H |
| 7183 | acetyl | H | H | H | H | acetyl | H | H | H | acetyl | acetyl | acetyl |
| 7184 | acetyl | H | H | H | H | acetyl | H | H | acetyl | H | H | H |
| 7185 | acetyl | H | H | H | H | acetyl | H | H | acetyl | H | H | acetyl |
| 7186 | acetyl | H | H | H | H | acetyl | H | H | acetyl | H | acetyl | H |
| 7187 | acetyl | H | H | H | H | acetyl | H | H | acetyl | H | acetyl | acetyl |
| 7188 | acetyl | H | H | H | H | acetyl | H | H | acetyl | acetyl | H | H |
| 7189 | acetyl | H | H | H | H | acetyl | H | H | acetyl | acetyl | H | acetyl |
| 7190 | acetyl | H | H | H | H | acetyl | H | H | acetyl | acetyl | acetyl | H |
| 7191 | acetyl | H | H | H | H | acetyl | H | H | acetyl | acetyl | acetyl | acetyl |
| 7192 | acetyl | H | H | H | H | acetyl | H | acetyl | H | H | H | H |
| 7193 | acetyl | H | H | H | H | acetyl | H | acetyl | H | H | H | acetyl |
| 7194 | acetyl | H | H | H | H | acetyl | H | acetyl | H | H | acetyl | H |
| 7195 | acetyl | H | H | H | H | acetyl | H | acetyl | H | H | acetyl | acetyl |
| 7196 | acetyl | H | H | H | H | acetyl | H | acetyl | H | acetyl | H | H |
| 7197 | acetyl | H | H | H | H | acetyl | H | acetyl | H | acetyl | H | acetyl |
| 7198 | acetyl | H | H | H | H | acetyl | H | acetyl | H | acetyl | acetyl | H |
| 7199 | acetyl | H | H | H | H | acetyl | H | acetyl | H | acetyl | acetyl | acetyl |
| 7200 | acetyl | H | H | H | H | acetyl | H | acetyl | acetyl | H | H | H |
| 7201 | acetyl | H | H | H | H | acetyl | H | acetyl | acetyl | H | H | acetyl |
| 7202 | acetyl | H | H | H | H | acetyl | H | acetyl | acetyl | H | acetyl | H |
| 7203 | acetyl | H | H | H | H | acetyl | H | acetyl | acetyl | H | acetyl | acetyl |
| 7204 | acetyl | H | H | H | H | acetyl | H | acetyl | acetyl | acetyl | H | H |
| 7205 | acetyl | H | H | H | H | acetyl | H | acetyl | acetyl | acetyl | H | acetyl |
| 7206 | acetyl | H | H | H | H | acetyl | H | acetyl | acetyl | acetyl | acetyl | H |
| 7207 | acetyl | H | H | H | H | acetyl | H | acetyl | acetyl | acetyl | acetyl | acetyl |
| 7208 | acetyl | H | H | H | H | acetyl | acetyl | H | H | H | H | H |
| 7209 | acetyl | H | H | H | H | acetyl | acetyl | H | H | H | H | acetyl |
| 7210 | acetyl | H | H | H | H | acetyl | acetyl | H | H | H | acetyl | H |
| 7211 | acetyl | H | H | H | H | acetyl | acetyl | H | H | H | acetyl | acetyl |
| 7212 | acetyl | H | H | H | H | acetyl | acetyl | H | H | acetyl | H | H |
| 7213 | acetyl | H | H | H | H | acetyl | acetyl | H | H | acetyl | H | acetyl |
| 7214 | acetyl | H | H | H | H | acetyl | acetyl | H | H | acetyl | acetyl | H |
| 7215 | acetyl | H | H | H | H | acetyl | acetyl | H | H | acetyl | acetyl | acetyl |
| 7216 | acetyl | H | H | H | H | acetyl | acetyl | H | acetyl | H | H | H |
| 7217 | acetyl | H | H | H | H | acetyl | acetyl | H | acetyl | H | H | acetyl |
| 7218 | acetyl | H | H | H | H | acetyl | acetyl | H | acetyl | H | acetyl | H |
| 7219 | acetyl | H | H | H | H | acetyl | acetyl | H | acetyl | H | acetyl | acetyl |
| 7220 | acetyl | H | H | H | H | acetyl | acetyl | H | acetyl | acetyl | H | H |
| 7221 | acetyl | H | H | H | H | acetyl | acetyl | H | acetyl | acetyl | H | acetyl |
| 7222 | acetyl | H | H | H | H | acetyl | acetyl | H | acetyl | acetyl | acetyl | H |
| 7223 | acetyl | H | H | H | H | acetyl | acetyl | H | acetyl | acetyl | acetyl | acetyl |
| 7224 | acetyl | H | H | H | H | acetyl | acetyl | acetyl | H | H | H | H |
| 7225 | acetyl | H | H | H | H | acetyl | acetyl | acetyl | H | H | H | acetyl |
| 7226 | acetyl | H | H | H | H | acetyl | acetyl | acetyl | H | H | acetyl | H |
| 7227 | acetyl | H | H | H | H | acetyl | acetyl | acetyl | H | H | acetyl | acetyl |
| 7228 | acetyl | H | H | H | H | acetyl | acetyl | acetyl | H | acetyl | H | H |
| 7229 | acetyl | H | H | H | H | acetyl | acetyl | acetyl | H | acetyl | H | acetyl |
| 7230 | acetyl | H | H | H | H | acetyl | acetyl | acetyl | H | acetyl | acetyl | H |
| 7231 | acetyl | H | H | H | H | acetyl | acetyl | acetyl | H | acetyl | acetyl | acetyl |
| 7232 | acetyl | H | H | H | H | acetyl | acetyl | acetyl | acetyl | H | H | H |
| 7233 | acetyl | H | H | H | H | acetyl | acetyl | acetyl | acetyl | H | H | acetyl |
| 7234 | acetyl | H | H | H | H | acetyl | acetyl | acetyl | acetyl | H | acetyl | H |
| 7235 | acetyl | H | H | H | H | acetyl | acetyl | acetyl | acetyl | H | acetyl | acetyl |
| 7236 | acetyl | H | H | H | H | acetyl | acetyl | acetyl | acetyl | acetyl | H | H |
| 7237 | acetyl | H | H | H | H | acetyl | acetyl | acetyl | acetyl | acetyl | H | acetyl |
| 7238 | acetyl | H | H | H | H | acetyl | acetyl | acetyl | acetyl | acetyl | acetyl | H |
| 7239 | acetyl | H | H | H | H | acetyl | acetyl | acetyl | acetyl | acetyl | acetyl | acetyl |
| 7240 | acetyl | H | H | H | acetyl | H | H | H | H | H | H | H |
| 7241 | acetyl | H | H | H | acetyl | H | H | H | H | H | H | acetyl |
| 7242 | acetyl | H | H | H | acetyl | H | H | H | H | H | acetyl | H |
| 7243 | acetyl | H | H | H | acetyl | H | H | H | H | H | acetyl | acetyl |
| 7244 | acetyl | H | H | H | acetyl | H | H | H | H | acetyl | H | H |
| 7245 | acetyl | H | H | H | acetyl | H | H | H | H | acetyl | H | acetyl |
| 7246 | acetyl | H | H | H | acetyl | H | H | H | H | acetyl | acetyl | H |
| 7247 | acetyl | H | H | H | acetyl | H | H | H | H | acetyl | acetyl | acetyl |
| 7248 | acetyl | H | H | H | acetyl | H | H | H | acetyl | H | H | H |
| 7249 | acetyl | H | H | H | acetyl | H | H | H | acetyl | H | H | acetyl |
| 7250 | acetyl | H | H | H | acetyl | H | H | H | acetyl | H | acetyl | H |
| 7251 | acetyl | H | H | H | acetyl | H | H | H | acetyl | H | acetyl | acetyl |
| 7252 | acetyl | H | H | H | acetyl | H | H | H | acetyl | acetyl | H | H |
| 7253 | acetyl | H | H | H | acetyl | H | H | H | acetyl | acetyl | H | acetyl |
| 7254 | acetyl | H | H | H | acetyl | H | H | H | acetyl | acetyl | acetyl | H |
| 7255 | acetyl | H | H | H | acetyl | H | H | H | acetyl | acetyl | acetyl | acetyl |
| 7256 | acetyl | H | H | H | acetyl | H | H | acetyl | H | H | H | H |
| 7257 | acetyl | H | H | H | acetyl | H | H | acetyl | H | H | H | acetyl |
| 7258 | acetyl | H | H | H | acetyl | H | H | acetyl | H | H | acetyl | H |
| 7259 | acetyl | H | H | H | acetyl | H | H | acetyl | H | H | acetyl | acetyl |
| 7260 | acetyl | H | H | H | acetyl | H | H | acetyl | H | acetyl | H | H |
| 7261 | acetyl | H | H | H | acetyl | H | H | acetyl | H | acetyl | H | acetyl |
| 7262 | acetyl | H | H | H | acetyl | H | H | acetyl | H | acetyl | acetyl | H |
| 7263 | acetyl | H | H | H | acetyl | H | H | acetyl | H | acetyl | acetyl | acetyl |
| 7264 | acetyl | H | H | H | acetyl | H | H | acetyl | acetyl | H | H | H |
| 7265 | acetyl | H | H | H | acetyl | H | H | acetyl | acetyl | H | H | acetyl |
| 7266 | acetyl | H | H | H | acetyl | H | H | acetyl | acetyl | H | acetyl | H |
| 7267 | acetyl | H | H | H | acetyl | H | H | acetyl | acetyl | H | acetyl | acetyl |
| 7268 | acetyl | H | H | H | acetyl | H | H | acetyl | acetyl | acetyl | H | H |
| 7269 | acetyl | H | H | H | acetyl | H | H | acetyl | acetyl | acetyl | H | acetyl |
| 7270 | acetyl | H | H | H | acetyl | H | H | acetyl | acetyl | acetyl | acetyl | H |
| 7271 | acetyl | H | H | H | acetyl | H | H | acetyl | acetyl | acetyl | acetyl | acetyl |
| 7272 | acetyl | H | H | H | acetyl | H | acetyl | H | H | H | H | H |
| 7273 | acetyl | H | H | H | acetyl | H | acetyl | H | H | H | H | acetyl |
| 7274 | acetyl | H | H | H | acetyl | H | acetyl | H | H | H | acetyl | H |
| 7275 | acetyl | H | H | H | acetyl | H | acetyl | H | H | H | acetyl | acetyl |
| 7276 | acetyl | H | H | H | acetyl | H | acetyl | H | H | acetyl | H | H |
| 7277 | acetyl | H | H | H | acetyl | H | acetyl | H | H | acetyl | H | acetyl |
| 7278 | acetyl | H | H | H | acetyl | H | acetyl | H | H | acetyl | acetyl | H |
| 7279 | acetyl | H | H | H | acetyl | H | acetyl | H | H | acetyl | acetyl | acetyl |
| 7280 | acetyl | H | H | H | acetyl | H | acetyl | H | acetyl | H | H | H |
| 7281 | acetyl | H | H | H | acetyl | H | acetyl | H | acetyl | H | H | acetyl |
| 7282 | acetyl | H | H | H | acetyl | H | acetyl | H | acetyl | H | acetyl | H |
| 7283 | acetyl | H | H | H | acetyl | H | acetyl | H | acetyl | H | acetyl | acetyl |
| 7284 | acetyl | H | H | H | acetyl | H | acetyl | H | acetyl | acetyl | H | H |
| 7285 | acetyl | H | H | H | acetyl | H | acetyl | H | acetyl | acetyl | H | acetyl |
| 7286 | acetyl | H | H | H | acetyl | H | acetyl | H | acetyl | acetyl | acetyl | H |
| 7287 | acetyl | H | H | H | acetyl | H | acetyl | H | acetyl | acetyl | acetyl | acetyl |
| 7288 | acetyl | H | H | H | acetyl | H | acetyl | acetyl | H | H | H | H |
| 7289 | acetyl | H | H | H | acetyl | H | acetyl | acetyl | H | H | H | acetyl |
| 7290 | acetyl | H | H | H | acetyl | H | acetyl | acetyl | H | H | acetyl | H |
| 7291 | acetyl | H | H | H | acetyl | H | acetyl | acetyl | H | H | acetyl | acetyl |
| 7292 | acetyl | H | H | H | acetyl | H | acetyl | acetyl | H | acetyl | H | H |
| 7293 | acetyl | H | H | H | acetyl | H | acetyl | acetyl | H | acetyl | H | acetyl |
| 7294 | acetyl | H | H | H | acetyl | H | acetyl | acetyl | H | acetyl | acetyl | H |
| 7295 | acetyl | H | H | H | acetyl | H | acetyl | acetyl | H | acetyl | acetyl | acetyl |
| 7296 | acetyl | H | H | H | acetyl | H | acetyl | acetyl | acetyl | H | H | H |
| 7297 | acetyl | H | H | H | acetyl | H | acetyl | acetyl | acetyl | H | H | acetyl |
| 7298 | acetyl | H | H | H | acetyl | H | acetyl | acetyl | acetyl | H | acetyl | H |
| 7299 | acetyl | H | H | H | acetyl | H | acetyl | acetyl | acetyl | H | acetyl | acetyl |
| 7300 | acetyl | H | H | H | acetyl | H | acetyl | acetyl | acetyl | acetyl | H | H |
| 7301 | acetyl | H | H | H | acetyl | H | acetyl | acetyl | acetyl | acetyl | H | acetyl |
| 7302 | acetyl | H | H | H | acetyl | H | acetyl | acetyl | acetyl | acetyl | acetyl | H |
| 7303 | acetyl | H | H | H | acetyl | H | acetyl | acetyl | acetyl | acetyl | acetyl | acetyl |
| 7304 | acetyl | H | H | H | acetyl | acetyl | H | H | H | H | H | H |
| 7305 | acetyl | H | H | H | acetyl | acetyl | H | H | H | H | H | acetyl |
| 7306 | acetyl | H | H | H | acetyl | acetyl | H | H | H | H | acetyl | H |
| 7307 | acetyl | H | H | H | acetyl | acetyl | H | H | H | H | acetyl | acetyl |
| 7308 | acetyl | H | H | H | acetyl | acetyl | H | H | H | acetyl | H | H |
| 7309 | acetyl | H | H | H | acetyl | acetyl | H | H | H | acetyl | H | acetyl |
| 7310 | acetyl | H | H | H | acetyl | acetyl | H | H | H | acetyl | acetyl | H |
| 7311 | acetyl | H | H | H | acetyl | acetyl | H | H | H | acetyl | acetyl | acetyl |
| 7312 | acetyl | H | H | H | acetyl | acetyl | H | H | acetyl | H | H | H |
| 7313 | acetyl | H | H | H | acetyl | acetyl | H | H | acetyl | H | H | acetyl |
| 7314 | acetyl | H | H | H | acetyl | acetyl | H | H | acetyl | H | acetyl | H |
| 7315 | acetyl | H | H | H | acetyl | acetyl | H | H | acetyl | H | acetyl | acetyl |
| 7316 | acetyl | H | H | H | acetyl | acetyl | H | H | acetyl | acetyl | H | H |
| 7317 | acetyl | H | H | H | acetyl | acetyl | H | H | acetyl | acetyl | H | acetyl |
| 7318 | acetyl | H | H | H | acetyl | acetyl | H | H | acetyl | acetyl | acetyl | H |
| 7319 | acetyl | H | H | H | acetyl | acetyl | H | H | acetyl | acetyl | acetyl | acetyl |
| 7320 | acetyl | H | H | H | acetyl | acetyl | H | acetyl | H | H | H | H |
| 7321 | acetyl | H | H | H | acetyl | acetyl | H | acetyl | H | H | H | acetyl |
| 7322 | acetyl | H | H | H | acetyl | acetyl | H | acetyl | H | H | acetyl | H |
| 7323 | acetyl | H | H | H | acetyl | acetyl | H | acetyl | H | H | acetyl | acetyl |
| 7324 | acetyl | H | H | H | acetyl | acetyl | H | acetyl | H | acetyl | H | H |
| 7325 | acetyl | H | H | H | acetyl | acetyl | H | acetyl | H | acetyl | H | acetyl |
| 7326 | acetyl | H | H | H | acetyl | acetyl | H | acetyl | H | acetyl | acetyl | H |
| 7327 | acetyl | H | H | H | acetyl | acetyl | H | acetyl | H | acetyl | acetyl | acetyl |
| 7328 | acetyl | H | H | H | acetyl | acetyl | H | acetyl | acetyl | H | H | H |
| 7329 | acetyl | H | H | H | acetyl | acetyl | H | acetyl | acetyl | H | H | acetyl |
| 7330 | acetyl | H | H | H | acetyl | acetyl | H | acetyl | acetyl | H | acetyl | H |
| 7331 | acetyl | H | H | H | acetyl | acetyl | H | acetyl | acetyl | H | acetyl | acetyl |
| 7332 | acetyl | H | H | H | acetyl | acetyl | H | acetyl | acetyl | acetyl | H | H |
| 7333 | acetyl | H | H | H | acetyl | acetyl | H | acetyl | acetyl | acetyl | H | acetyl |
| 7334 | acetyl | H | H | H | acetyl | acetyl | H | acetyl | acetyl | acetyl | acetyl | H |
| 7335 | acetyl | H | H | H | acetyl | acetyl | H | acetyl | acetyl | acetyl | acetyl | acetyl |
| 7336 | acetyl | H | H | H | acetyl | acetyl | acetyl | H | H | H | H | H |
| 7337 | acetyl | H | H | H | acetyl | acetyl | acetyl | H | H | H | H | acetyl |
| 7338 | acetyl | H | H | H | acetyl | acetyl | acetyl | H | H | H | acetyl | H |
| 7339 | acetyl | H | H | H | acetyl | acetyl | acetyl | H | H | H | acetyl | acetyl |
| 7340 | acetyl | H | H | H | acetyl | acetyl | acetyl | H | H | acetyl | H | H |
| 7341 | acetyl | H | H | H | acetyl | acetyl | acetyl | H | H | acetyl | H | acetyl |
| 7342 | acetyl | H | H | H | acetyl | acetyl | acetyl | H | H | acetyl | acetyl | H |
| 7343 | acetyl | H | H | H | acetyl | acetyl | acetyl | H | H | acetyl | acetyl | acetyl |
| 7344 | acetyl | H | H | H | acetyl | acetyl | acetyl | H | acetyl | H | H | H |
| 7345 | acetyl | H | H | H | acetyl | acetyl | acetyl | H | acetyl | H | H | acetyl |
| 7346 | acetyl | H | H | H | acetyl | acetyl | acetyl | H | acetyl | H | acetyl | H |
| 7347 | acetyl | H | H | H | acetyl | acetyl | acetyl | H | acetyl | H | acetyl | acetyl |
| 7348 | acetyl | H | H | H | acetyl | acetyl | acetyl | H | acetyl | acetyl | H | H |
| 7349 | acetyl | H | H | H | acetyl | acetyl | acetyl | H | acetyl | acetyl | H | acetyl |
| 7350 | acetyl | H | H | H | acetyl | acetyl | acetyl | H | acetyl | acetyl | acetyl | H |
| 7351 | acetyl | H | H | H | acetyl | acetyl | acetyl | H | acetyl | acetyl | acetyl | acetyl |
| 7352 | acetyl | H | H | H | acetyl | acetyl | acetyl | acetyl | H | H | H | H |
| 7353 | acetyl | H | H | H | acetyl | acetyl | acetyl | acetyl | H | H | H | acetyl |
| 7354 | acetyl | H | H | H | acetyl | acetyl | acetyl | acetyl | H | H | acetyl | H |
| 7355 | acetyl | H | H | H | acetyl | acetyl | acetyl | acetyl | H | H | acetyl | acetyl |
| 7356 | acetyl | H | H | H | acetyl | acetyl | acetyl | acetyl | H | acetyl | H | H |
| 7357 | acetyl | H | H | H | acetyl | acetyl | acetyl | acetyl | H | acetyl | H | acetyl |
| 7358 | acetyl | H | H | H | acetyl | acetyl | acetyl | acetyl | H | acetyl | acetyl | H |
| 7359 | acetyl | H | H | H | acetyl | acetyl | acetyl | acetyl | H | acetyl | acetyl | acetyl |
| 7360 | acetyl | H | H | H | acetyl | acetyl | acetyl | acetyl | acetyl | H | H | H |
| 7361 | acetyl | H | H | H | acetyl | acetyl | acetyl | acetyl | acetyl | H | H | acetyl |
| 7362 | acetyl | H | H | H | acetyl | acetyl | acetyl | acetyl | acetyl | H | acetyl | H |
| 7363 | acetyl | H | H | H | acetyl | acetyl | acetyl | acetyl | acetyl | H | acetyl | acetyl |
| 7364 | acetyl | H | H | H | acetyl | acetyl | acetyl | acetyl | acetyl | acetyl | H | H |
| 7365 | acetyl | H | H | H | acetyl | acetyl | acetyl | acetyl | acetyl | acetyl | H | acetyl |
| 7366 | acetyl | H | H | H | acetyl | acetyl | acetyl | acetyl | acetyl | acetyl | acetyl | H |
| 7367 | acetyl | H | H | H | acetyl | acetyl | acetyl | acetyl | acetyl | acetyl | acetyl | acetyl |
| 7368 | acetyl | H | H | acetyl | H | H | H | H | H | H | H | H |
| 7369 | acetyl | H | H | acetyl | H | H | H | H | H | H | H | acetyl |
| 7370 | acetyl | H | H | acetyl | H | H | H | H | H | H | acetyl | H |
| 7371 | acetyl | H | H | acetyl | H | H | H | H | H | H | acetyl | acetyl |
| 7372 | acetyl | H | H | acetyl | H | H | H | H | H | acetyl | H | H |
| 7373 | acetyl | H | H | acetyl | H | H | H | H | H | acetyl | H | acetyl |
| 7374 | acetyl | H | H | acetyl | H | H | H | H | H | acetyl | acetyl | H |
| 7375 | acetyl | H | H | acetyl | H | H | H | H | H | acetyl | acetyl | acetyl |
| 7376 | acetyl | H | H | acetyl | H | H | H | H | acetyl | H | H | H |
| 7377 | acetyl | H | H | acetyl | H | H | H | H | acetyl | H | H | acetyl |
| 7378 | acetyl | H | H | acetyl | H | H | H | H | acetyl | H | acetyl | H |
| 7379 | acetyl | H | H | acetyl | H | H | H | H | acetyl | H | acetyl | acetyl |
| 7380 | acetyl | H | H | acetyl | H | H | H | H | acetyl | acetyl | H | H |
| 7381 | acetyl | H | H | acetyl | H | H | H | H | acetyl | acetyl | H | acetyl |
| 7382 | acetyl | H | H | acetyl | H | H | H | H | acetyl | acetyl | acetyl | H |
| 7383 | acetyl | H | H | acetyl | H | H | H | H | acetyl | acetyl | acetyl | acetyl |
| 7384 | acetyl | H | H | acetyl | H | H | H | acetyl | H | H | H | H |
| 7385 | acetyl | H | H | acetyl | H | H | H | acetyl | H | H | H | acetyl |
| 7386 | acetyl | H | H | acetyl | H | H | H | acetyl | H | H | acetyl | H |
| 7387 | acetyl | H | H | acetyl | H | H | H | acetyl | H | H | acetyl | acetyl |
| 7388 | acetyl | H | H | acetyl | H | H | H | acetyl | H | acetyl | H | H |
| 7389 | acetyl | H | H | acetyl | H | H | H | acetyl | H | acetyl | H | acetyl |
| 7390 | acetyl | H | H | acetyl | H | H | H | acetyl | H | acetyl | acetyl | H |
| 7391 | acetyl | H | H | acetyl | H | H | H | acetyl | H | acetyl | acetyl | acetyl |
| 7392 | acetyl | H | H | acetyl | H | H | H | acetyl | acetyl | H | H | H |
| 7393 | acetyl | H | H | acetyl | H | H | H | acetyl | acetyl | H | H | acetyl |
| 7394 | acetyl | H | H | acetyl | H | H | H | acetyl | acetyl | H | acetyl | H |
| 7395 | acetyl | H | H | acetyl | H | H | H | acetyl | acetyl | H | acetyl | acetyl |
| 7396 | acetyl | H | H | acetyl | H | H | H | acetyl | acetyl | acetyl | H | H |
| 7397 | acetyl | H | H | acetyl | H | H | H | acetyl | acetyl | acetyl | H | acetyl |
| 7398 | acetyl | H | H | acetyl | H | H | H | acetyl | acetyl | acetyl | acetyl | H |
| 7399 | acetyl | H | H | acetyl | H | H | H | acetyl | acetyl | acetyl | acetyl | acetyl |
| 7400 | acetyl | H | H | acetyl | H | H | acetyl | H | H | H | H | H |
| 7401 | acetyl | H | H | acetyl | H | H | acetyl | H | H | H | H | acetyl |
| 7402 | acetyl | H | H | acetyl | H | H | acetyl | H | H | H | acetyl | H |
| 7403 | acetyl | H | H | acetyl | H | H | acetyl | H | H | H | acetyl | acetyl |
| 7404 | acetyl | H | H | acetyl | H | H | acetyl | H | H | acetyl | H | H |
| 7405 | acetyl | H | H | acetyl | H | H | acetyl | H | H | acetyl | H | acetyl |
| 7406 | acetyl | H | H | acetyl | H | H | acetyl | H | H | acetyl | acetyl | H |
| 7407 | acetyl | H | H | acetyl | H | H | acetyl | H | H | acetyl | acetyl | acetyl |
| 7408 | acetyl | H | H | acetyl | H | H | acetyl | H | acetyl | H | H | H |
| 7409 | acetyl | H | H | acetyl | H | H | acetyl | H | acetyl | H | H | acetyl |
| 7410 | acetyl | H | H | acetyl | H | H | acetyl | H | acetyl | H | acetyl | H |
| 7411 | acetyl | H | H | acetyl | H | H | acetyl | H | acetyl | H | acetyl | acetyl |
| 7412 | acetyl | H | H | acetyl | H | H | acetyl | H | acetyl | acetyl | H | H |
| 7413 | acetyl | H | H | acetyl | H | H | acetyl | H | acetyl | acetyl | H | acetyl |
| 7414 | acetyl | H | H | acetyl | H | H | acetyl | H | acetyl | acetyl | acetyl | H |
| 7415 | acetyl | H | H | acetyl | H | H | acetyl | H | acetyl | acetyl | acetyl | acetyl |
| 7416 | acetyl | H | H | acetyl | H | H | acetyl | acetyl | H | H | H | H |
| 7417 | acetyl | H | H | acetyl | H | H | acetyl | acetyl | H | H | H | acetyl |
| 7418 | acetyl | H | H | acetyl | H | H | acetyl | acetyl | H | H | acetyl | H |
| 7419 | acetyl | H | H | acetyl | H | H | acetyl | acetyl | H | H | acetyl | acetyl |
| 7420 | acetyl | H | H | acetyl | H | H | acetyl | acetyl | H | acetyl | H | H |
| 7421 | acetyl | H | H | acetyl | H | H | acetyl | acetyl | H | acetyl | H | acetyl |
| 7422 | acetyl | H | H | acetyl | H | H | acetyl | acetyl | H | acetyl | acetyl | H |
| 7423 | acetyl | H | H | acetyl | H | H | acetyl | acetyl | H | acetyl | acetyl | acetyl |
| 7424 | acetyl | H | H | acetyl | H | H | acetyl | acetyl | acetyl | H | H | H |
| 7425 | acetyl | H | H | acetyl | H | H | acetyl | acetyl | acetyl | H | H | acetyl |
| 7426 | acetyl | H | H | acetyl | H | H | acetyl | acetyl | acetyl | H | acetyl | H |
| 7427 | acetyl | H | H | acetyl | H | H | acetyl | acetyl | acetyl | H | acetyl | acetyl |
| 7428 | acetyl | H | H | acetyl | H | H | acetyl | acetyl | acetyl | acetyl | H | H |
| 7429 | acetyl | H | H | acetyl | H | H | acetyl | acetyl | acetyl | acetyl | H | acetyl |
| 7430 | acetyl | H | H | acetyl | H | H | acetyl | acetyl | acetyl | acetyl | acetyl | H |
| 7431 | acetyl | H | H | acetyl | H | H | acetyl | acetyl | acetyl | acetyl | acetyl | acetyl |
| 7432 | acetyl | H | H | acetyl | H | acetyl | H | H | H | H | H | H |
| 7433 | acetyl | H | H | acetyl | H | acetyl | H | H | H | H | H | acetyl |
| 7434 | acetyl | H | H | acetyl | H | acetyl | H | H | H | H | acetyl | H |
| 7435 | acetyl | H | H | acetyl | H | acetyl | H | H | H | H | acetyl | acetyl |
| 7436 | acetyl | H | H | acetyl | H | acetyl | H | H | H | acetyl | H | H |
| 7437 | acetyl | H | H | acetyl | H | acetyl | H | H | H | acetyl | H | acetyl |
| 7438 | acetyl | H | H | acetyl | H | acetyl | H | H | H | acetyl | acetyl | H |
| 7439 | acetyl | H | H | acetyl | H | acetyl | H | H | H | acetyl | acetyl | acetyl |
| 7440 | acetyl | H | H | acetyl | H | acetyl | H | H | acetyl | H | H | H |
| 7441 | acetyl | H | H | acetyl | H | acetyl | H | H | acetyl | H | H | acetyl |
| 7442 | acetyl | H | H | acetyl | H | acetyl | H | H | acetyl | H | acetyl | H |
| 7443 | acetyl | H | H | acetyl | H | acetyl | H | H | acetyl | H | acetyl | acetyl |
| 7444 | acetyl | H | H | acetyl | H | acetyl | H | H | acetyl | acetyl | H | H |
| 7445 | acetyl | H | H | acetyl | H | acetyl | H | H | acetyl | acetyl | H | acetyl |
| 7446 | acetyl | H | H | acetyl | H | acetyl | H | H | acetyl | acetyl | acetyl | H |
| 7447 | acetyl | H | H | acetyl | H | acetyl | H | H | acetyl | acetyl | acetyl | acetyl |
| 7448 | acetyl | H | H | acetyl | H | acetyl | H | acetyl | H | H | H | H |
| 7449 | acetyl | H | H | acetyl | H | acetyl | H | acetyl | H | H | H | acetyl |
| 7450 | acetyl | H | H | acetyl | H | acetyl | H | acetyl | H | H | acetyl | H |
| 7451 | acetyl | H | H | acetyl | H | acetyl | H | acetyl | H | H | acetyl | acetyl |
| 7452 | acetyl | H | H | acetyl | H | acetyl | H | acetyl | H | acetyl | H | H |
| 7453 | acetyl | H | H | acetyl | H | acetyl | H | acetyl | H | acetyl | H | acetyl |
| 7454 | acetyl | H | H | acetyl | H | acetyl | H | acetyl | H | acetyl | acetyl | H |
| 7455 | acetyl | H | H | acetyl | H | acetyl | H | acetyl | H | acetyl | acetyl | acetyl |
| 7456 | acetyl | H | H | acetyl | H | acetyl | H | acetyl | acetyl | H | H | H |
| 7457 | acetyl | H | H | acetyl | H | acetyl | H | acetyl | acetyl | H | H | acetyl |
| 7458 | acetyl | H | H | acetyl | H | acetyl | H | acetyl | acetyl | H | acetyl | H |
| 7459 | acetyl | H | H | acetyl | H | acetyl | H | acetyl | acetyl | H | acetyl | acetyl |
| 7460 | acetyl | H | H | acetyl | H | acetyl | H | acetyl | acetyl | acetyl | H | H |
| 7461 | acetyl | H | H | acetyl | H | acetyl | H | acetyl | acetyl | acetyl | H | acetyl |
| 7462 | acetyl | H | H | acetyl | H | acetyl | H | acetyl | acetyl | acetyl | acetyl | H |
| 7463 | acetyl | H | H | acetyl | H | acetyl | H | acetyl | acetyl | acetyl | acetyl | acetyl |
| 7464 | acetyl | H | H | acetyl | H | acetyl | acetyl | H | H | H | H | H |
| 7465 | acetyl | H | H | acetyl | H | acetyl | acetyl | H | H | H | H | acetyl |
| 7466 | acetyl | H | H | acetyl | H | acetyl | acetyl | H | H | H | acetyl | H |
| 7467 | acetyl | H | H | acetyl | H | acetyl | acetyl | H | H | H | acetyl | acetyl |
| 7468 | acetyl | H | H | acetyl | H | acetyl | acetyl | H | H | acetyl | H | H |
| 7469 | acetyl | H | H | acetyl | H | acetyl | acetyl | H | H | acetyl | H | acetyl |
| 7470 | acetyl | H | H | acetyl | H | acetyl | acetyl | H | H | acetyl | acetyl | H |
| 7471 | acetyl | H | H | acetyl | H | acetyl | acetyl | H | H | acetyl | acetyl | acetyl |
| 7472 | acetyl | H | H | acetyl | H | acetyl | acetyl | H | acetyl | H | H | H |
| 7473 | acetyl | H | H | acetyl | H | acetyl | acetyl | H | acetyl | H | H | acetyl |
| 7474 | acetyl | H | H | acetyl | H | acetyl | acetyl | H | acetyl | H | acetyl | H |
| 7475 | acetyl | H | H | acetyl | H | acetyl | acetyl | H | acetyl | H | acetyl | acetyl |
| 7476 | acetyl | H | H | acetyl | H | acetyl | acetyl | H | acetyl | acetyl | H | H |
| 7477 | acetyl | H | H | acetyl | H | acetyl | acetyl | H | acetyl | acetyl | H | acetyl |
| 7478 | acetyl | H | H | acetyl | H | acetyl | acetyl | H | acetyl | acetyl | acetyl | H |
| 7479 | acetyl | H | H | acetyl | H | acetyl | acetyl | H | acetyl | acetyl | acetyl | acetyl |
| 7480 | acetyl | H | H | acetyl | H | acetyl | acetyl | acetyl | H | H | H | H |
| 7481 | acetyl | H | H | acetyl | H | acetyl | acetyl | acetyl | H | H | H | acetyl |
| 7482 | acetyl | H | H | acetyl | H | acetyl | acetyl | acetyl | H | H | acetyl | H |
| 7483 | acetyl | H | H | acetyl | H | acetyl | acetyl | acetyl | H | H | acetyl | acetyl |
| 7484 | acetyl | H | H | acetyl | H | acetyl | acetyl | acetyl | H | acetyl | H | H |
| 7485 | acetyl | H | H | acetyl | H | acetyl | acetyl | acetyl | H | acetyl | H | acetyl |
| 7486 | acetyl | H | H | acetyl | H | acetyl | acetyl | acetyl | H | acetyl | acetyl | H |
| 7487 | acetyl | H | H | acetyl | H | acetyl | acetyl | acetyl | H | acetyl | acetyl | acetyl |
| 7488 | acetyl | H | H | acetyl | H | acetyl | acetyl | acetyl | acetyl | H | H | H |
| 7489 | acetyl | H | H | acetyl | H | acetyl | acetyl | acetyl | acetyl | H | H | acetyl |
| 7490 | acetyl | H | H | acetyl | H | acetyl | acetyl | acetyl | acetyl | H | acetyl | H |
| 7491 | acetyl | H | H | acetyl | H | acetyl | acetyl | acetyl | acetyl | H | acetyl | acetyl |
| 7492 | acetyl | H | H | acetyl | H | acetyl | acetyl | acetyl | acetyl | acetyl | H | H |
| 7493 | acetyl | H | H | acetyl | H | acetyl | acetyl | acetyl | acetyl | acetyl | H | acetyl |
| 7494 | acetyl | H | H | acetyl | H | acetyl | acetyl | acetyl | acetyl | acetyl | acetyl | H |
| 7495 | acetyl | H | H | acetyl | H | acetyl | acetyl | acetyl | acetyl | acetyl | acetyl | acetyl |
| 7496 | acetyl | H | H | acetyl | acetyl | H | H | H | H | H | H | H |
| 7497 | acetyl | H | H | acetyl | acetyl | H | H | H | H | H | H | acetyl |
| 7498 | acetyl | H | H | acetyl | acetyl | H | H | H | H | H | acetyl | H |
| 7499 | acetyl | H | H | acetyl | acetyl | H | H | H | H | H | acetyl | acetyl |
| 7500 | acetyl | H | H | acetyl | acetyl | H | H | H | H | acetyl | H | H |
| 7501 | acetyl | H | H | acetyl | acetyl | H | H | H | H | acetyl | H | acetyl |
| 7502 | acetyl | H | H | acetyl | acetyl | H | H | H | H | acetyl | acetyl | H |
| 7503 | acetyl | H | H | acetyl | acetyl | H | H | H | H | acetyl | acetyl | acetyl |
| 7504 | acetyl | H | H | acetyl | acetyl | H | H | H | acetyl | H | H | H |
| 7505 | acetyl | H | H | acetyl | acetyl | H | H | H | acetyl | H | H | acetyl |
| 7506 | acetyl | H | H | acetyl | acetyl | H | H | H | acetyl | H | acetyl | H |
| 7507 | acetyl | H | H | acetyl | acetyl | H | H | H | acetyl | H | acetyl | acetyl |
| 7508 | acetyl | H | H | acetyl | acetyl | H | H | H | acetyl | acetyl | H | H |
| 7509 | acetyl | H | H | acetyl | acetyl | H | H | H | acetyl | acetyl | H | acetyl |
| 7510 | acetyl | H | H | acetyl | acetyl | H | H | H | acetyl | acetyl | acetyl | H |
| 7511 | acetyl | H | H | acetyl | acetyl | H | H | H | acetyl | acetyl | acetyl | acetyl |
| 7512 | acetyl | H | H | acetyl | acetyl | H | H | acetyl | H | H | H | H |
| 7513 | acetyl | H | H | acetyl | acetyl | H | H | acetyl | H | H | H | acetyl |
| 7514 | acetyl | H | H | acetyl | acetyl | H | H | acetyl | H | H | acetyl | H |
| 7515 | acetyl | H | H | acetyl | acetyl | H | H | acetyl | H | H | acetyl | acetyl |
| 7516 | acetyl | H | H | acetyl | acetyl | H | H | acetyl | H | acetyl | H | H |
| 7517 | acetyl | H | H | acetyl | acetyl | H | H | acetyl | H | acetyl | H | acetyl |
| 7518 | acetyl | H | H | acetyl | acetyl | H | H | acetyl | H | acetyl | acetyl | H |
| 7519 | acetyl | H | H | acetyl | acetyl | H | H | acetyl | H | acetyl | acetyl | acetyl |
| 7520 | acetyl | H | H | acetyl | acetyl | H | H | acetyl | acetyl | H | H | H |
| 7521 | acetyl | H | H | acetyl | acetyl | H | H | acetyl | acetyl | H | H | acetyl |
| 7522 | acetyl | H | H | acetyl | acetyl | H | H | acetyl | acetyl | H | acetyl | H |
| 7523 | acetyl | H | H | acetyl | acetyl | H | H | acetyl | acetyl | H | acetyl | acetyl |
| 7524 | acetyl | H | H | acetyl | acetyl | H | H | acetyl | acetyl | acetyl | H | H |
| 7525 | acetyl | H | H | acetyl | acetyl | H | H | acetyl | acetyl | acetyl | H | acetyl |
| 7526 | acetyl | H | H | acetyl | acetyl | H | H | acetyl | acetyl | acetyl | acetyl | H |
| 7527 | acetyl | H | H | acetyl | acetyl | H | H | acetyl | acetyl | acetyl | acetyl | acetyl |
| 7528 | acetyl | H | H | acetyl | acetyl | H | acetyl | H | H | H | H | H |
| 7529 | acetyl | H | H | acetyl | acetyl | H | acetyl | H | H | H | H | acetyl |
| 7530 | acetyl | H | H | acetyl | acetyl | H | acetyl | H | H | H | acetyl | H |
| 7531 | acetyl | H | H | acetyl | acetyl | H | acetyl | H | H | H | acetyl | acetyl |
| 7532 | acetyl | H | H | acetyl | acetyl | H | acetyl | H | H | acetyl | H | H |
| 7533 | acetyl | H | H | acetyl | acetyl | H | acetyl | H | H | acetyl | H | acetyl |
| 7534 | acetyl | H | H | acetyl | acetyl | H | acetyl | H | H | acetyl | acetyl | H |
| 7535 | acetyl | H | H | acetyl | acetyl | H | acetyl | H | H | acetyl | acetyl | acetyl |
| 7536 | acetyl | H | H | acetyl | acetyl | H | acetyl | H | acetyl | H | H | H |
| 7537 | acetyl | H | H | acetyl | acetyl | H | acetyl | H | acetyl | H | H | acetyl |
| 7538 | acetyl | H | H | acetyl | acetyl | H | acetyl | H | acetyl | H | acetyl | H |
| 7539 | acetyl | H | H | acetyl | acetyl | H | acetyl | H | acetyl | H | acetyl | acetyl |
| 7540 | acetyl | H | H | acetyl | acetyl | H | acetyl | H | acetyl | acetyl | H | H |
| 7541 | acetyl | H | H | acetyl | acetyl | H | acetyl | H | acetyl | acetyl | H | acetyl |
| 7542 | acetyl | H | H | acetyl | acetyl | H | acetyl | H | acetyl | acetyl | acetyl | H |
| 7543 | acetyl | H | H | acetyl | acetyl | H | acetyl | H | acetyl | acetyl | acetyl | acetyl |
| 7544 | acetyl | H | H | acetyl | acetyl | H | acetyl | acetyl | H | H | H | H |
| 7545 | acetyl | H | H | acetyl | acetyl | H | acetyl | acetyl | H | H | H | acetyl |
| 7546 | acetyl | H | H | acetyl | acetyl | H | acetyl | acetyl | H | H | acetyl | H |
| 7547 | acetyl | H | H | acetyl | acetyl | H | acetyl | acetyl | H | H | acetyl | acetyl |
| 7548 | acetyl | H | H | acetyl | acetyl | H | acetyl | acetyl | H | acetyl | H | H |
| 7549 | acetyl | H | H | acetyl | acetyl | H | acetyl | acetyl | H | acetyl | H | acetyl |
| 7550 | acetyl | H | H | acetyl | acetyl | H | acetyl | acetyl | H | acetyl | acetyl | H |
| 7551 | acetyl | H | H | acetyl | acetyl | H | acetyl | acetyl | H | acetyl | acetyl | acetyl |
| 7552 | acetyl | H | H | acetyl | acetyl | H | acetyl | acetyl | acetyl | H | H | H |
| 7553 | acetyl | H | H | acetyl | acetyl | H | acetyl | acetyl | acetyl | H | H | acetyl |
| 7554 | acetyl | H | H | acetyl | acetyl | H | acetyl | acetyl | acetyl | H | acetyl | H |
| 7555 | acetyl | H | H | acetyl | acetyl | H | acetyl | acetyl | acetyl | H | acetyl | acetyl |
| 7556 | acetyl | H | H | acetyl | acetyl | H | acetyl | acetyl | acetyl | acetyl | H | H |
| 7557 | acetyl | H | H | acetyl | acetyl | H | acetyl | acetyl | acetyl | acetyl | H | acetyl |
| 7558 | acetyl | H | H | acetyl | acetyl | H | acetyl | acetyl | acetyl | acetyl | acetyl | H |
| 7559 | acetyl | H | H | acetyl | acetyl | H | acetyl | acetyl | acetyl | acetyl | acetyl | acetyl |
| 7560 | acetyl | H | H | acetyl | acetyl | acetyl | H | H | H | H | H | H |
| 7561 | acetyl | H | H | acetyl | acetyl | acetyl | H | H | H | H | H | acetyl |
| 7562 | acetyl | H | H | acetyl | acetyl | acetyl | H | H | H | H | acetyl | H |
| 7563 | acetyl | H | H | acetyl | acetyl | acetyl | H | H | H | H | acetyl | acetyl |
| 7564 | acetyl | H | H | acetyl | acetyl | acetyl | H | H | H | acetyl | H | H |
| 7565 | acetyl | H | H | acetyl | acetyl | acetyl | H | H | H | acetyl | H | acetyl |
| 7566 | acetyl | H | H | acetyl | acetyl | acetyl | H | H | H | acetyl | acetyl | H |
| 7567 | acetyl | H | H | acetyl | acetyl | acetyl | H | H | H | acetyl | acetyl | acetyl |
| 7568 | acetyl | H | H | acetyl | acetyl | acetyl | H | H | acetyl | H | H | H |
| 7569 | acetyl | H | H | acetyl | acetyl | acetyl | H | H | acetyl | H | H | acetyl |
| 7570 | acetyl | H | H | acetyl | acetyl | acetyl | H | H | acetyl | H | acetyl | H |
| 7571 | acetyl | H | H | acetyl | acetyl | acetyl | H | H | acetyl | H | acetyl | acetyl |
| 7572 | acetyl | H | H | acetyl | acetyl | acetyl | H | H | acetyl | acetyl | H | H |
| 7573 | acetyl | H | H | acetyl | acetyl | acetyl | H | H | acetyl | acetyl | H | acetyl |
| 7574 | acetyl | H | H | acetyl | acetyl | acetyl | H | H | acetyl | acetyl | acetyl | H |
| 7575 | acetyl | H | H | acetyl | acetyl | acetyl | H | H | acetyl | acetyl | acetyl | acetyl |
| 7576 | acetyl | H | H | acetyl | acetyl | acetyl | H | acetyl | H | H | H | H |
| 7577 | acetyl | H | H | acetyl | acetyl | acetyl | H | acetyl | H | H | H | acetyl |
| 7578 | acetyl | H | H | acetyl | acetyl | acetyl | H | acetyl | H | H | acetyl | H |
| 7579 | acetyl | H | H | acetyl | acetyl | acetyl | H | acetyl | H | H | acetyl | acetyl |
| 7580 | acetyl | H | H | acetyl | acetyl | acetyl | H | acetyl | H | acetyl | H | H |
| 7581 | acetyl | H | H | acetyl | acetyl | acetyl | H | acetyl | H | acetyl | H | acetyl |
| 7582 | acetyl | H | H | acetyl | acetyl | acetyl | H | acetyl | H | acetyl | acetyl | H |
| 7583 | acetyl | H | H | acetyl | acetyl | acetyl | H | acetyl | H | acetyl | acetyl | acetyl |
| 7584 | acetyl | H | H | acetyl | acetyl | acetyl | H | acetyl | acetyl | H | H | H |
| 7585 | acetyl | H | H | acetyl | acetyl | acetyl | H | acetyl | acetyl | H | H | acetyl |
| 7586 | acetyl | H | H | acetyl | acetyl | acetyl | H | acetyl | acetyl | H | acetyl | H |
| 7587 | acetyl | H | H | acetyl | acetyl | acetyl | H | acetyl | acetyl | H | acetyl | acetyl |
| 7588 | acetyl | H | H | acetyl | acetyl | acetyl | H | acetyl | acetyl | acetyl | H | H |
| 7589 | acetyl | H | H | acetyl | acetyl | acetyl | H | acetyl | acetyl | acetyl | H | acetyl |
| 7590 | acetyl | H | H | acetyl | acetyl | acetyl | H | acetyl | acetyl | acetyl | acetyl | H |
| 7591 | acetyl | H | H | acetyl | acetyl | acetyl | H | acetyl | acetyl | acetyl | acetyl | acetyl |
| 7592 | acetyl | H | H | acetyl | acetyl | acetyl | acetyl | H | H | H | H | H |
| 7593 | acetyl | H | H | acetyl | acetyl | acetyl | acetyl | H | H | H | H | acetyl |
| 7594 | acetyl | H | H | acetyl | acetyl | acetyl | acetyl | H | H | H | acetyl | H |
| 7595 | acetyl | H | H | acetyl | acetyl | acetyl | acetyl | H | H | H | acetyl | acetyl |
| 7596 | acetyl | H | H | acetyl | acetyl | acetyl | acetyl | H | H | acetyl | H | H |
| 7597 | acetyl | H | H | acetyl | acetyl | acetyl | acetyl | H | H | acetyl | H | acetyl |
| 7598 | acetyl | H | H | acetyl | acetyl | acetyl | acetyl | H | H | acetyl | acetyl | H |
| 7599 | acetyl | H | H | acetyl | acetyl | acetyl | acetyl | H | H | acetyl | acetyl | acetyl |
| 7600 | acetyl | H | H | acetyl | acetyl | acetyl | acetyl | H | acetyl | H | H | H |
| 7601 | acetyl | H | H | acetyl | acetyl | acetyl | acetyl | H | acetyl | H | H | acetyl |
| 7602 | acetyl | H | H | acetyl | acetyl | acetyl | acetyl | H | acetyl | H | acetyl | H |
| 7603 | acetyl | H | H | acetyl | acetyl | acetyl | acetyl | H | acetyl | H | acetyl | acetyl |
| 7604 | acetyl | H | H | acetyl | acetyl | acetyl | acetyl | H | acetyl | acetyl | H | H |
| 7605 | acetyl | H | H | acetyl | acetyl | acetyl | acetyl | H | acetyl | acetyl | H | acetyl |
| 7606 | acetyl | H | H | acetyl | acetyl | acetyl | acetyl | H | acetyl | acetyl | acetyl | H |
| 7607 | acetyl | H | H | acetyl | acetyl | acetyl | acetyl | H | acetyl | acetyl | acetyl | acetyl |
| 7608 | acetyl | H | H | acetyl | acetyl | acetyl | acetyl | acetyl | H | H | H | H |
| 7609 | acetyl | H | H | acetyl | acetyl | acetyl | acetyl | acetyl | H | H | H | acetyl |
| 7610 | acetyl | H | H | acetyl | acetyl | acetyl | acetyl | acetyl | H | H | acetyl | H |
| 7611 | acetyl | H | H | acetyl | acetyl | acetyl | acetyl | acetyl | H | H | acetyl | acetyl |
| 7612 | acetyl | H | H | acetyl | acetyl | acetyl | acetyl | acetyl | H | acetyl | H | H |
| 7613 | acetyl | H | H | acetyl | acetyl | acetyl | acetyl | acetyl | H | acetyl | H | acetyl |
| 7614 | acetyl | H | H | acetyl | acetyl | acetyl | acetyl | acetyl | H | acetyl | acetyl | H |
| 7615 | acetyl | H | H | acetyl | acetyl | acetyl | acetyl | acetyl | H | acetyl | acetyl | acetyl |
| 7616 | acetyl | H | H | acetyl | acetyl | acetyl | acetyl | acetyl | acetyl | H | H | H |
| 7617 | acetyl | H | H | acetyl | acetyl | acetyl | acetyl | acetyl | acetyl | H | H | acetyl |
| 7618 | acetyl | H | H | acetyl | acetyl | acetyl | acetyl | acetyl | acetyl | H | acetyl | H |
| 7619 | acetyl | H | H | acetyl | acetyl | acetyl | acetyl | acetyl | acetyl | H | acetyl | acetyl |
| 7620 | acetyl | H | H | acetyl | acetyl | acetyl | acetyl | acetyl | acetyl | acetyl | H | H |
| 7621 | acetyl | H | H | acetyl | acetyl | acetyl | acetyl | acetyl | acetyl | acetyl | H | acetyl |
| 7622 | acetyl | H | H | acetyl | acetyl | acetyl | acetyl | acetyl | acetyl | acetyl | acetyl | H |
| 7623 | acetyl | H | H | acetyl | acetyl | acetyl | acetyl | acetyl | acetyl | acetyl | acetyl | acetyl |
| 7624 | acetyl | H | acetyl | H | H | H | H | H | H | H | H | H |
| 7625 | acetyl | H | acetyl | H | H | H | H | H | H | H | H | acetyl |
| 7626 | acetyl | H | acetyl | H | H | H | H | H | H | H | acetyl | H |
| 7627 | acetyl | H | acetyl | H | H | H | H | H | H | H | acetyl | acetyl |
| 7628 | acetyl | H | acetyl | H | H | H | H | H | H | acetyl | H | H |
| 7629 | acetyl | H | acetyl | H | H | H | H | H | H | acetyl | H | acetyl |
| 7630 | acetyl | H | acetyl | H | H | H | H | H | H | acetyl | acetyl | H |
| 7631 | acetyl | H | acetyl | H | H | H | H | H | H | acetyl | acetyl | acetyl |
| 7632 | acetyl | H | acetyl | H | H | H | H | H | acetyl | H | H | H |
| 7633 | acetyl | H | acetyl | H | H | H | H | H | acetyl | H | H | acetyl |
| 7634 | acetyl | H | acetyl | H | H | H | H | H | acetyl | H | acetyl | H |
| 7635 | acetyl | H | acetyl | H | H | H | H | H | acetyl | H | acetyl | acetyl |
| 7636 | acetyl | H | acetyl | H | H | H | H | H | acetyl | acetyl | H | H |
| 7637 | acetyl | H | acetyl | H | H | H | H | H | acetyl | acetyl | H | acetyl |
| 7638 | acetyl | H | acetyl | H | H | H | H | H | acetyl | acetyl | acetyl | H |
| 7639 | acetyl | H | acetyl | H | H | H | H | H | acetyl | acetyl | acetyl | acetyl |
| 7640 | acetyl | H | acetyl | H | H | H | H | acetyl | H | H | H | H |
| 7641 | acetyl | H | acetyl | H | H | H | H | acetyl | H | H | H | acetyl |
| 7642 | acetyl | H | acetyl | H | H | H | H | acetyl | H | H | acetyl | H |
| 7643 | acetyl | H | acetyl | H | H | H | H | acetyl | H | H | acetyl | acetyl |
| 7644 | acetyl | H | acetyl | H | H | H | H | acetyl | H | acetyl | H | H |
| 7645 | acetyl | H | acetyl | H | H | H | H | acetyl | H | acetyl | H | acetyl |
| 7646 | acetyl | H | acetyl | H | H | H | H | acetyl | H | acetyl | acetyl | H |
| 7647 | acetyl | H | acetyl | H | H | H | H | acetyl | H | acetyl | acetyl | acetyl |
| 7648 | acetyl | H | acetyl | H | H | H | H | acetyl | acetyl | H | H | H |
| 7649 | acetyl | H | acetyl | H | H | H | H | acetyl | acetyl | H | H | acetyl |
| 7650 | acetyl | H | acetyl | H | H | H | H | acetyl | acetyl | H | acetyl | H |
| 7651 | acetyl | H | acetyl | H | H | H | H | acetyl | acetyl | H | acetyl | acetyl |
| 7652 | acetyl | H | acetyl | H | H | H | H | acetyl | acetyl | acetyl | H | H |
| 7653 | acetyl | H | acetyl | H | H | H | H | acetyl | acetyl | acetyl | H | acetyl |
| 7654 | acetyl | H | acetyl | H | H | H | H | acetyl | acetyl | acetyl | acetyl | H |
| 7655 | acetyl | H | acetyl | H | H | H | H | acetyl | acetyl | acetyl | acetyl | acetyl |
| 7656 | acetyl | H | acetyl | H | H | H | acetyl | H | H | H | H | H |
| 7657 | acetyl | H | acetyl | H | H | H | acetyl | H | H | H | H | acetyl |
| 7658 | acetyl | H | acetyl | H | H | H | acetyl | H | H | H | acetyl | H |
| 7659 | acetyl | H | acetyl | H | H | H | acetyl | H | H | H | acetyl | acetyl |
| 7660 | acetyl | H | acetyl | H | H | H | acetyl | H | H | acetyl | H | H |
| 7661 | acetyl | H | acetyl | H | H | H | acetyl | H | H | acetyl | H | acetyl |
| 7662 | acetyl | H | acetyl | H | H | H | acetyl | H | H | acetyl | acetyl | H |
| 7663 | acetyl | H | acetyl | H | H | H | acetyl | H | H | acetyl | acetyl | acetyl |
| 7664 | acetyl | H | acetyl | H | H | H | acetyl | H | acetyl | H | H | H |
| 7665 | acetyl | H | acetyl | H | H | H | acetyl | H | acetyl | H | H | acetyl |
| 7666 | acetyl | H | acetyl | H | H | H | acetyl | H | acetyl | H | acetyl | H |
| 7667 | acetyl | H | acetyl | H | H | H | acetyl | H | acetyl | H | acetyl | acetyl |
| 7668 | acetyl | H | acetyl | H | H | H | acetyl | H | acetyl | acetyl | H | H |
| 7669 | acetyl | H | acetyl | H | H | H | acetyl | H | acetyl | acetyl | H | acetyl |
| 7670 | acetyl | H | acetyl | H | H | H | acetyl | H | acetyl | acetyl | acetyl | H |
| 7671 | acetyl | H | acetyl | H | H | H | acetyl | H | acetyl | acetyl | acetyl | acetyl |
| 7672 | acetyl | H | acetyl | H | H | H | acetyl | acetyl | H | H | H | H |
| 7673 | acetyl | H | acetyl | H | H | H | acetyl | acetyl | H | H | H | acetyl |
| 7674 | acetyl | H | acetyl | H | H | H | acetyl | acetyl | H | H | acetyl | H |
| 7675 | acetyl | H | acetyl | H | H | H | acetyl | acetyl | H | H | acetyl | acetyl |
| 7676 | acetyl | H | acetyl | H | H | H | acetyl | acetyl | H | acetyl | H | H |
| 7677 | acetyl | H | acetyl | H | H | H | acetyl | acetyl | H | acetyl | H | acetyl |
| 7678 | acetyl | H | acetyl | H | H | H | acetyl | acetyl | H | acetyl | acetyl | H |
| 7679 | acetyl | H | acetyl | H | H | H | acetyl | acetyl | H | acetyl | acetyl | acetyl |
| 7680 | acetyl | H | acetyl | H | H | H | acetyl | acetyl | acetyl | H | H | H |
| 7681 | acetyl | H | acetyl | H | H | H | acetyl | acetyl | acetyl | H | H | acetyl |
| 7682 | acetyl | H | acetyl | H | H | H | acetyl | acetyl | acetyl | H | acetyl | H |
| 7683 | acetyl | H | acetyl | H | H | H | acetyl | acetyl | acetyl | H | acetyl | acetyl |
| 7684 | acetyl | H | acetyl | H | H | H | acetyl | acetyl | acetyl | acetyl | H | H |
| 7685 | acetyl | H | acetyl | H | H | H | acetyl | acetyl | acetyl | acetyl | H | acetyl |
| 7686 | acetyl | H | acetyl | H | H | H | acetyl | acetyl | acetyl | acetyl | acetyl | H |
| 7687 | acetyl | H | acetyl | H | H | H | acetyl | acetyl | acetyl | acetyl | acetyl | acetyl |
| 7688 | acetyl | H | acetyl | H | H | acetyl | H | H | H | H | H | H |
| 7689 | acetyl | H | acetyl | H | H | acetyl | H | H | H | H | H | acetyl |
| 7690 | acetyl | H | acetyl | H | H | acetyl | H | H | H | H | acetyl | H |
| 7691 | acetyl | H | acetyl | H | H | acetyl | H | H | H | H | acetyl | acetyl |
| 7692 | acetyl | H | acetyl | H | H | acetyl | H | H | H | acetyl | H | H |
| 7693 | acetyl | H | acetyl | H | H | acetyl | H | H | H | acetyl | H | acetyl |
| 7694 | acetyl | H | acetyl | H | H | acetyl | H | H | H | acetyl | acetyl | H |
| 7695 | acetyl | H | acetyl | H | H | acetyl | H | H | H | acetyl | acetyl | acetyl |
| 7696 | acetyl | H | acetyl | H | H | acetyl | H | H | acetyl | H | H | H |
| 7697 | acetyl | H | acetyl | H | H | acetyl | H | H | acetyl | H | H | acetyl |
| 7698 | acetyl | H | acetyl | H | H | acetyl | H | H | acetyl | H | acetyl | H |
| 7699 | acetyl | H | acetyl | H | H | acetyl | H | H | acetyl | H | acetyl | acetyl |
| 7700 | acetyl | H | acetyl | H | H | acetyl | H | H | acetyl | acetyl | H | H |
| 7701 | acetyl | H | acetyl | H | H | acetyl | H | H | acetyl | acetyl | H | acetyl |
| 7702 | acetyl | H | acetyl | H | H | acetyl | H | H | acetyl | acetyl | acetyl | H |
| 7703 | acetyl | H | acetyl | H | H | acetyl | H | H | acetyl | acetyl | acetyl | acetyl |
| 7704 | acetyl | H | acetyl | H | H | acetyl | H | acetyl | H | H | H | H |
| 7705 | acetyl | H | acetyl | H | H | acetyl | H | acetyl | H | H | H | acetyl |
| 7706 | acetyl | H | acetyl | H | H | acetyl | H | acetyl | H | H | acetyl | H |
| 7707 | acetyl | H | acetyl | H | H | acetyl | H | acetyl | H | H | acetyl | acetyl |
| 7708 | acetyl | H | acetyl | H | H | acetyl | H | acetyl | H | acetyl | H | H |
| 7709 | acetyl | H | acetyl | H | H | acetyl | H | acetyl | H | acetyl | H | acetyl |
| 7710 | acetyl | H | acetyl | H | H | acetyl | H | acetyl | H | acetyl | acetyl | H |
| 7711 | acetyl | H | acetyl | H | H | acetyl | H | acetyl | H | acetyl | acetyl | acetyl |
| 7712 | acetyl | H | acetyl | H | H | acetyl | H | acetyl | acetyl | H | H | H |
| 7713 | acetyl | H | acetyl | H | H | acetyl | H | acetyl | acetyl | H | H | acetyl |
| 7714 | acetyl | H | acetyl | H | H | acetyl | H | acetyl | acetyl | H | acetyl | H |
| 7715 | acetyl | H | acetyl | H | H | acetyl | H | acetyl | acetyl | H | acetyl | acetyl |
| 7716 | acetyl | H | acetyl | H | H | acetyl | H | acetyl | acetyl | acetyl | H | H |
| 7717 | acetyl | H | acetyl | H | H | acetyl | H | acetyl | acetyl | acetyl | H | acetyl |
| 7718 | acetyl | H | acetyl | H | H | acetyl | H | acetyl | acetyl | acetyl | acetyl | H |
| 7719 | acetyl | H | acetyl | H | H | acetyl | H | acetyl | acetyl | acetyl | acetyl | acetyl |
| 7720 | acetyl | H | acetyl | H | H | acetyl | acetyl | H | H | H | H | H |
| 7721 | acetyl | H | acetyl | H | H | acetyl | acetyl | H | H | H | H | acetyl |
| 7722 | acetyl | H | acetyl | H | H | acetyl | acetyl | H | H | H | acetyl | H |
| 7723 | acetyl | H | acetyl | H | H | acetyl | acetyl | H | H | H | acetyl | acetyl |
| 7724 | acetyl | H | acetyl | H | H | acetyl | acetyl | H | H | acetyl | H | H |
| 7725 | acetyl | H | acetyl | H | H | acetyl | acetyl | H | H | acetyl | H | acetyl |
| 7726 | acetyl | H | acetyl | H | H | acetyl | acetyl | H | H | acetyl | acetyl | H |
| 7727 | acetyl | H | acetyl | H | H | acetyl | acetyl | H | H | acetyl | acetyl | acetyl |
| 7728 | acetyl | H | acetyl | H | H | acetyl | acetyl | H | acetyl | H | H | H |
| 7729 | acetyl | H | acetyl | H | H | acetyl | acetyl | H | acetyl | H | H | acetyl |
| 7730 | acetyl | H | acetyl | H | H | acetyl | acetyl | H | acetyl | H | acetyl | H |
| 7731 | acetyl | H | acetyl | H | H | acetyl | acetyl | H | acetyl | H | acetyl | acetyl |
| 7732 | acetyl | H | acetyl | H | H | acetyl | acetyl | H | acetyl | acetyl | H | H |
| 7733 | acetyl | H | acetyl | H | H | acetyl | acetyl | H | acetyl | acetyl | H | acetyl |
| 7734 | acetyl | H | acetyl | H | H | acetyl | acetyl | H | acetyl | acetyl | acetyl | H |
| 7735 | acetyl | H | acetyl | H | H | acetyl | acetyl | H | acetyl | acetyl | acetyl | acetyl |
| 7736 | acetyl | H | acetyl | H | H | acetyl | acetyl | acetyl | H | H | H | H |
| 7737 | acetyl | H | acetyl | H | H | acetyl | acetyl | acetyl | H | H | H | acetyl |
| 7738 | acetyl | H | acetyl | H | H | acetyl | acetyl | acetyl | H | H | acetyl | H |
| 7739 | acetyl | H | acetyl | H | H | acetyl | acetyl | acetyl | H | H | acetyl | acetyl |
| 7740 | acetyl | H | acetyl | H | H | acetyl | acetyl | acetyl | H | acetyl | H | H |
| 7741 | acetyl | H | acetyl | H | H | acetyl | acetyl | acetyl | H | acetyl | H | acetyl |
| 7742 | acetyl | H | acetyl | H | H | acetyl | acetyl | acetyl | H | acetyl | acetyl | H |
| 7743 | acetyl | H | acetyl | H | H | acetyl | acetyl | acetyl | H | acetyl | acetyl | acetyl |
| 7744 | acetyl | H | acetyl | H | H | acetyl | acetyl | acetyl | acetyl | H | H | H |
| 7745 | acetyl | H | acetyl | H | H | acetyl | acetyl | acetyl | acetyl | H | H | acetyl |
| 7746 | acetyl | H | acetyl | H | H | acetyl | acetyl | acetyl | acetyl | H | acetyl | H |
| 7747 | acetyl | H | acetyl | H | H | acetyl | acetyl | acetyl | acetyl | H | acetyl | acetyl |
| 7748 | acetyl | H | acetyl | H | H | acetyl | acetyl | acetyl | acetyl | acetyl | H | H |
| 7749 | acetyl | H | acetyl | H | H | acetyl | acetyl | acetyl | acetyl | acetyl | H | acetyl |
| 7750 | acetyl | H | acetyl | H | H | acetyl | acetyl | acetyl | acetyl | acetyl | acetyl | H |
| 7751 | acetyl | H | acetyl | H | H | acetyl | acetyl | acetyl | acetyl | acetyl | acetyl | acetyl |
| 7752 | acetyl | H | acetyl | H | acetyl | H | H | H | H | H | H | H |
| 7753 | acetyl | H | acetyl | H | acetyl | H | H | H | H | H | H | acetyl |
| 7754 | acetyl | H | acetyl | H | acetyl | H | H | H | H | H | acetyl | H |
| 7755 | acetyl | H | acetyl | H | acetyl | H | H | H | H | H | acetyl | acetyl |
| 7756 | acetyl | H | acetyl | H | acetyl | H | H | H | H | acetyl | H | H |
| 7757 | acetyl | H | acetyl | H | acetyl | H | H | H | H | acetyl | H | acetyl |
| 7758 | acetyl | H | acetyl | H | acetyl | H | H | H | H | acetyl | acetyl | H |
| 7759 | acetyl | H | acetyl | H | acetyl | H | H | H | H | acetyl | acetyl | acetyl |
| 7760 | acetyl | H | acetyl | H | acetyl | H | H | H | acetyl | H | H | H |
| 7761 | acetyl | H | acetyl | H | acetyl | H | H | H | acetyl | H | H | acetyl |
| 7762 | acetyl | H | acetyl | H | acetyl | H | H | H | acetyl | H | acetyl | H |
| 7763 | acetyl | H | acetyl | H | acetyl | H | H | H | acetyl | H | acetyl | acetyl |
| 7764 | acetyl | H | acetyl | H | acetyl | H | H | H | acetyl | acetyl | H | H |
| 7765 | acetyl | H | acetyl | H | acetyl | H | H | H | acetyl | acetyl | H | acetyl |
| 7766 | acetyl | H | acetyl | H | acetyl | H | H | H | acetyl | acetyl | acetyl | H |
| 7767 | acetyl | H | acetyl | H | acetyl | H | H | H | acetyl | acetyl | acetyl | acetyl |
| 7768 | acetyl | H | acetyl | H | acetyl | H | H | acetyl | H | H | H | H |
| 7769 | acetyl | H | acetyl | H | acetyl | H | H | acetyl | H | H | H | acetyl |
| 7770 | acetyl | H | acetyl | H | acetyl | H | H | acetyl | H | H | acetyl | H |
| 7771 | acetyl | H | acetyl | H | acetyl | H | H | acetyl | H | H | acetyl | acetyl |
| 7772 | acetyl | H | acetyl | H | acetyl | H | H | acetyl | H | acetyl | H | H |
| 7773 | acetyl | H | acetyl | H | acetyl | H | H | acetyl | H | acetyl | H | acetyl |
| 7774 | acetyl | H | acetyl | H | acetyl | H | H | acetyl | H | acetyl | acetyl | H |
| 7775 | acetyl | H | acetyl | H | acetyl | H | H | acetyl | H | acetyl | acetyl | acetyl |
| 7776 | acetyl | H | acetyl | H | acetyl | H | H | acetyl | acetyl | H | H | H |
| 7777 | acetyl | H | acetyl | H | acetyl | H | H | acetyl | acetyl | H | H | acetyl |
| 7778 | acetyl | H | acetyl | H | acetyl | H | H | acetyl | acetyl | H | acetyl | H |
| 7779 | acetyl | H | acetyl | H | acetyl | H | H | acetyl | acetyl | H | acetyl | acetyl |
| 7780 | acetyl | H | acetyl | H | acetyl | H | H | acetyl | acetyl | acetyl | H | H |
| 7781 | acetyl | H | acetyl | H | acetyl | H | H | acetyl | acetyl | acetyl | H | acetyl |
| 7782 | acetyl | H | acetyl | H | acetyl | H | H | acetyl | acetyl | acetyl | acetyl | H |
| 7783 | acetyl | H | acetyl | H | acetyl | H | H | acetyl | acetyl | acetyl | acetyl | acetyl |
| 7784 | acetyl | H | acetyl | H | acetyl | H | acetyl | H | H | H | H | H |
| 7785 | acetyl | H | acetyl | H | acetyl | H | acetyl | H | H | H | H | acetyl |
| 7786 | acetyl | H | acetyl | H | acetyl | H | acetyl | H | H | H | acetyl | H |
| 7787 | acetyl | H | acetyl | H | acetyl | H | acetyl | H | H | H | acetyl | acetyl |
| 7788 | acetyl | H | acetyl | H | acetyl | H | acetyl | H | H | acetyl | H | H |
| 7789 | acetyl | H | acetyl | H | acetyl | H | acetyl | H | H | acetyl | H | acetyl |
| 7790 | acetyl | H | acetyl | H | acetyl | H | acetyl | H | H | acetyl | acetyl | H |
| 7791 | acetyl | H | acetyl | H | acetyl | H | acetyl | H | H | acetyl | acetyl | acetyl |
| 7792 | acetyl | H | acetyl | H | acetyl | H | acetyl | H | acetyl | H | H | H |
| 7793 | acetyl | H | acetyl | H | acetyl | H | acetyl | H | acetyl | H | H | acetyl |
| 7794 | acetyl | H | acetyl | H | acetyl | H | acetyl | H | acetyl | H | acetyl | H |
| 7795 | acetyl | H | acetyl | H | acetyl | H | acetyl | H | acetyl | H | acetyl | acetyl |
| 7796 | acetyl | H | acetyl | H | acetyl | H | acetyl | H | acetyl | acetyl | H | H |
| 7797 | acetyl | H | acetyl | H | acetyl | H | acetyl | H | acetyl | acetyl | H | acetyl |
| 7798 | acetyl | H | acetyl | H | acetyl | H | acetyl | H | acetyl | acetyl | acetyl | H |
| 7799 | acetyl | H | acetyl | H | acetyl | H | acetyl | H | acetyl | acetyl | acetyl | acetyl |
| 7800 | acetyl | H | acetyl | H | acetyl | H | acetyl | acetyl | H | H | H | H |
| 7801 | acetyl | H | acetyl | H | acetyl | H | acetyl | acetyl | H | H | H | acetyl |
| 7802 | acetyl | H | acetyl | H | acetyl | H | acetyl | acetyl | H | H | acetyl | H |
| 7803 | acetyl | H | acetyl | H | acetyl | H | acetyl | acetyl | H | H | acetyl | acetyl |
| 7804 | acetyl | H | acetyl | H | acetyl | H | acetyl | acetyl | H | acetyl | H | H |
| 7805 | acetyl | H | acetyl | H | acetyl | H | acetyl | acetyl | H | acetyl | H | acetyl |
| 7806 | acetyl | H | acetyl | H | acetyl | H | acetyl | acetyl | H | acetyl | acetyl | H |
| 7807 | acetyl | H | acetyl | H | acetyl | H | acetyl | acetyl | H | acetyl | acetyl | acetyl |
| 7808 | acetyl | H | acetyl | H | acetyl | H | acetyl | acetyl | acetyl | H | H | H |
| 7809 | acetyl | H | acetyl | H | acetyl | H | acetyl | acetyl | acetyl | H | H | acetyl |
| 7810 | acetyl | H | acetyl | H | acetyl | H | acetyl | acetyl | acetyl | H | acetyl | H |
| 7811 | acetyl | H | acetyl | H | acetyl | H | acetyl | acetyl | acetyl | H | acetyl | acetyl |
| 7812 | acetyl | H | acetyl | H | acetyl | H | acetyl | acetyl | acetyl | acetyl | H | H |
| 7813 | acetyl | H | acetyl | H | acetyl | H | acetyl | acetyl | acetyl | acetyl | H | acetyl |
| 7814 | acetyl | H | acetyl | H | acetyl | H | acetyl | acetyl | acetyl | acetyl | acetyl | H |
| 7815 | acetyl | H | acetyl | H | acetyl | H | acetyl | acetyl | acetyl | acetyl | acetyl | acetyl |
| 7816 | acetyl | H | acetyl | H | acetyl | acetyl | H | H | H | H | H | H |
| 7817 | acetyl | H | acetyl | H | acetyl | acetyl | H | H | H | H | H | acetyl |
| 7818 | acetyl | H | acetyl | H | acetyl | acetyl | H | H | H | H | acetyl | H |
| 7819 | acetyl | H | acetyl | H | acetyl | acetyl | H | H | H | H | acetyl | acetyl |
| 7820 | acetyl | H | acetyl | H | acetyl | acetyl | H | H | H | acetyl | H | H |
| 7821 | acetyl | H | acetyl | H | acetyl | acetyl | H | H | H | acetyl | H | acetyl |
| 7822 | acetyl | H | acetyl | H | acetyl | acetyl | H | H | H | acetyl | acetyl | H |
| 7823 | acetyl | H | acetyl | H | acetyl | acetyl | H | H | H | acetyl | acetyl | acetyl |
| 7824 | acetyl | H | acetyl | H | acetyl | acetyl | H | H | acetyl | H | H | H |
| 7825 | acetyl | H | acetyl | H | acetyl | acetyl | H | H | acetyl | H | H | acetyl |
| 7826 | acetyl | H | acetyl | H | acetyl | acetyl | H | H | acetyl | H | acetyl | H |
| 7827 | acetyl | H | acetyl | H | acetyl | acetyl | H | H | acetyl | H | acetyl | acetyl |
| 7828 | acetyl | H | acetyl | H | acetyl | acetyl | H | H | acetyl | acetyl | H | H |
| 7829 | acetyl | H | acetyl | H | acetyl | acetyl | H | H | acetyl | acetyl | H | acetyl |
| 7830 | acetyl | H | acetyl | H | acetyl | acetyl | H | H | acetyl | acetyl | acetyl | H |
| 7831 | acetyl | H | acetyl | H | acetyl | acetyl | H | H | acetyl | acetyl | acetyl | acetyl |
| 7832 | acetyl | H | acetyl | H | acetyl | acetyl | H | acetyl | H | H | H | H |
| 7833 | acetyl | H | acetyl | H | acetyl | acetyl | H | acetyl | H | H | H | acetyl |
| 7834 | acetyl | H | acetyl | H | acetyl | acetyl | H | acetyl | H | H | acetyl | H |
| 7835 | acetyl | H | acetyl | H | acetyl | acetyl | H | acetyl | H | H | acetyl | acetyl |
| 7836 | acetyl | H | acetyl | H | acetyl | acetyl | H | acetyl | H | acetyl | H | H |
| 7837 | acetyl | H | acetyl | H | acetyl | acetyl | H | acetyl | H | acetyl | H | acetyl |
| 7838 | acetyl | H | acetyl | H | acetyl | acetyl | H | acetyl | H | acetyl | acetyl | H |
| 7839 | acetyl | H | acetyl | H | acetyl | acetyl | H | acetyl | H | acetyl | acetyl | acetyl |
| 7840 | acetyl | H | acetyl | H | acetyl | acetyl | H | acetyl | acetyl | H | H | H |
| 7841 | acetyl | H | acetyl | H | acetyl | acetyl | H | acetyl | acetyl | H | H | acetyl |
| 7842 | acetyl | H | acetyl | H | acetyl | acetyl | H | acetyl | acetyl | H | acetyl | H |
| 7843 | acetyl | H | acetyl | H | acetyl | acetyl | H | acetyl | acetyl | H | acetyl | acetyl |
| 7844 | acetyl | H | acetyl | H | acetyl | acetyl | H | acetyl | acetyl | acetyl | H | H |
| 7845 | acetyl | H | acetyl | H | acetyl | acetyl | H | acetyl | acetyl | acetyl | H | acetyl |
| 7846 | acetyl | H | acetyl | H | acetyl | acetyl | H | acetyl | acetyl | acetyl | acetyl | H |
| 7847 | acetyl | H | acetyl | H | acetyl | acetyl | H | acetyl | acetyl | acetyl | acetyl | acetyl |
| 7848 | acetyl | H | acetyl | H | acetyl | acetyl | acetyl | H | H | H | H | H |
| 7849 | acetyl | H | acetyl | H | acetyl | acetyl | acetyl | H | H | H | H | acetyl |
| 7850 | acetyl | H | acetyl | H | acetyl | acetyl | acetyl | H | H | H | acetyl | H |
| 7851 | acetyl | H | acetyl | H | acetyl | acetyl | acetyl | H | H | H | acetyl | acetyl |
| 7852 | acetyl | H | acetyl | H | acetyl | acetyl | acetyl | H | H | acetyl | H | H |
| 7853 | acetyl | H | acetyl | H | acetyl | acetyl | acetyl | H | H | acetyl | H | acetyl |
| 7854 | acetyl | H | acetyl | H | acetyl | acetyl | acetyl | H | H | acetyl | acetyl | H |
| 7855 | acetyl | H | acetyl | H | acetyl | acetyl | acetyl | H | H | acetyl | acetyl | acetyl |
| 7856 | acetyl | H | acetyl | H | acetyl | acetyl | acetyl | H | acetyl | H | H | H |
| 7857 | acetyl | H | acetyl | H | acetyl | acetyl | acetyl | H | acetyl | H | H | acetyl |
| 7858 | acetyl | H | acetyl | H | acetyl | acetyl | acetyl | H | acetyl | H | acetyl | H |
| 7859 | acetyl | H | acetyl | H | acetyl | acetyl | acetyl | H | acetyl | H | acetyl | acetyl |
| 7860 | acetyl | H | acetyl | H | acetyl | acetyl | acetyl | H | acetyl | acetyl | H | H |
| 7861 | acetyl | H | acetyl | H | acetyl | acetyl | acetyl | H | acetyl | acetyl | H | acetyl |
| 7862 | acetyl | H | acetyl | H | acetyl | acetyl | acetyl | H | acetyl | acetyl | acetyl | H |
| 7863 | acetyl | H | acetyl | H | acetyl | acetyl | acetyl | H | acetyl | acetyl | acetyl | acetyl |
| 7864 | acetyl | H | acetyl | H | acetyl | acetyl | acetyl | acetyl | H | H | H | H |
| 7865 | acetyl | H | acetyl | H | acetyl | acetyl | acetyl | acetyl | H | H | H | acetyl |
| 7866 | acetyl | H | acetyl | H | acetyl | acetyl | acetyl | acetyl | H | H | acetyl | H |
| 7867 | acetyl | H | acetyl | H | acetyl | acetyl | acetyl | acetyl | H | H | acetyl | acetyl |
| 7868 | acetyl | H | acetyl | H | acetyl | acetyl | acetyl | acetyl | H | acetyl | H | H |
| 7869 | acetyl | H | acetyl | H | acetyl | acetyl | acetyl | acetyl | H | acetyl | H | acetyl |
| 7870 | acetyl | H | acetyl | H | acetyl | acetyl | acetyl | acetyl | H | acetyl | acetyl | H |
| 7871 | acetyl | H | acetyl | H | acetyl | acetyl | acetyl | acetyl | H | acetyl | acetyl | acetyl |
| 7872 | acetyl | H | acetyl | H | acetyl | acetyl | acetyl | acetyl | acetyl | H | H | H |
| 7873 | acetyl | H | acetyl | H | acetyl | acetyl | acetyl | acetyl | acetyl | H | H | acetyl |
| 7874 | acetyl | H | acetyl | H | acetyl | acetyl | acetyl | acetyl | acetyl | H | acetyl | H |
| 7875 | acetyl | H | acetyl | H | acetyl | acetyl | acetyl | acetyl | acetyl | H | acetyl | acetyl |
| 7876 | acetyl | H | acetyl | H | acetyl | acetyl | acetyl | acetyl | acetyl | acetyl | H | H |
| 7877 | acetyl | H | acetyl | H | acetyl | acetyl | acetyl | acetyl | acetyl | acetyl | H | acetyl |
| 7878 | acetyl | H | acetyl | H | acetyl | acetyl | acetyl | acetyl | acetyl | acetyl | acetyl | H |
| 7879 | acetyl | H | acetyl | H | acetyl | acetyl | acetyl | acetyl | acetyl | acetyl | acetyl | acetyl |
| 7880 | acetyl | H | acetyl | acetyl | H | H | H | H | H | H | H | H |
| 7881 | acetyl | H | acetyl | acetyl | H | H | H | H | H | H | H | acetyl |
| 7882 | acetyl | H | acetyl | acetyl | H | H | H | H | H | H | acetyl | H |
| 7883 | acetyl | H | acetyl | acetyl | H | H | H | H | H | H | acetyl | acetyl |
| 7884 | acetyl | H | acetyl | acetyl | H | H | H | H | H | acetyl | H | H |
| 7885 | acetyl | H | acetyl | acetyl | H | H | H | H | H | acetyl | H | acetyl |
| 7886 | acetyl | H | acetyl | acetyl | H | H | H | H | H | acetyl | acetyl | H |
| 7887 | acetyl | H | acetyl | acetyl | H | H | H | H | H | acetyl | acetyl | acetyl |
| 7888 | acetyl | H | acetyl | acetyl | H | H | H | H | acetyl | H | H | H |
| 7889 | acetyl | H | acetyl | acetyl | H | H | H | H | acetyl | H | H | acetyl |
| 7890 | acetyl | H | acetyl | acetyl | H | H | H | H | acetyl | H | acetyl | H |
| 7891 | acetyl | H | acetyl | acetyl | H | H | H | H | acetyl | H | acetyl | acetyl |
| 7892 | acetyl | H | acetyl | acetyl | H | H | H | H | acetyl | acetyl | H | H |
| 7893 | acetyl | H | acetyl | acetyl | H | H | H | H | acetyl | acetyl | H | acetyl |
| 7894 | acetyl | H | acetyl | acetyl | H | H | H | H | acetyl | acetyl | acetyl | H |
| 7895 | acetyl | H | acetyl | acetyl | H | H | H | H | acetyl | acetyl | acetyl | acetyl |
| 7896 | acetyl | H | acetyl | acetyl | H | H | H | acetyl | H | H | H | H |
| 7897 | acetyl | H | acetyl | acetyl | H | H | H | acetyl | H | H | H | acetyl |
| 7898 | acetyl | H | acetyl | acetyl | H | H | H | acetyl | H | H | acetyl | H |
| 7899 | acetyl | H | acetyl | acetyl | H | H | H | acetyl | H | H | acetyl | acetyl |
| 7900 | acetyl | H | acetyl | acetyl | H | H | H | acetyl | H | acetyl | H | H |
| 7901 | acetyl | H | acetyl | acetyl | H | H | H | acetyl | H | acetyl | H | acetyl |
| 7902 | acetyl | H | acetyl | acetyl | H | H | H | acetyl | H | acetyl | acetyl | H |
| 7903 | acetyl | H | acetyl | acetyl | H | H | H | acetyl | H | acetyl | acetyl | acetyl |
| 7904 | acetyl | H | acetyl | acetyl | H | H | H | acetyl | acetyl | H | H | H |
| 7905 | acetyl | H | acetyl | acetyl | H | H | H | acetyl | acetyl | H | H | acetyl |
| 7906 | acetyl | H | acetyl | acetyl | H | H | H | acetyl | acetyl | H | acetyl | H |
| 7907 | acetyl | H | acetyl | acetyl | H | H | H | acetyl | acetyl | H | acetyl | acetyl |
| 7908 | acetyl | H | acetyl | acetyl | H | H | H | acetyl | acetyl | acetyl | H | H |
| 7909 | acetyl | H | acetyl | acetyl | H | H | H | acetyl | acetyl | acetyl | H | acetyl |
| 7910 | acetyl | H | acetyl | acetyl | H | H | H | acetyl | acetyl | acetyl | acetyl | H |
| 7911 | acetyl | H | acetyl | acetyl | H | H | H | acetyl | acetyl | acetyl | acetyl | acetyl |
| 7912 | acetyl | H | acetyl | acetyl | H | H | acetyl | H | H | H | H | H |
| 7913 | acetyl | H | acetyl | acetyl | H | H | acetyl | H | H | H | H | acetyl |
| 7914 | acetyl | H | acetyl | acetyl | H | H | acetyl | H | H | H | acetyl | H |
| 7915 | acetyl | H | acetyl | acetyl | H | H | acetyl | H | H | H | acetyl | acetyl |
| 7916 | acetyl | H | acetyl | acetyl | H | H | acetyl | H | H | acetyl | H | H |
| 7917 | acetyl | H | acetyl | acetyl | H | H | acetyl | H | H | acetyl | H | acetyl |
| 7918 | acetyl | H | acetyl | acetyl | H | H | acetyl | H | H | acetyl | acetyl | H |
| 7919 | acetyl | H | acetyl | acetyl | H | H | acetyl | H | H | acetyl | acetyl | acetyl |
| 7920 | acetyl | H | acetyl | acetyl | H | H | acetyl | H | acetyl | H | H | H |
| 7921 | acetyl | H | acetyl | acetyl | H | H | acetyl | H | acetyl | H | H | acetyl |
| 7922 | acetyl | H | acetyl | acetyl | H | H | acetyl | H | acetyl | H | acetyl | H |
| 7923 | acetyl | H | acetyl | acetyl | H | H | acetyl | H | acetyl | H | acetyl | acetyl |
| 7924 | acetyl | H | acetyl | acetyl | H | H | acetyl | H | acetyl | acetyl | H | H |
| 7925 | acetyl | H | acetyl | acetyl | H | H | acetyl | H | acetyl | acetyl | H | acetyl |
| 7926 | acetyl | H | acetyl | acetyl | H | H | acetyl | H | acetyl | acetyl | acetyl | H |
| 7927 | acetyl | H | acetyl | acetyl | H | H | acetyl | H | acetyl | acetyl | acetyl | acetyl |
| 7928 | acetyl | H | acetyl | acetyl | H | H | acetyl | acetyl | H | H | H | H |
| 7929 | acetyl | H | acetyl | acetyl | H | H | acetyl | acetyl | H | H | H | acetyl |
| 7930 | acetyl | H | acetyl | acetyl | H | H | acetyl | acetyl | H | H | acetyl | H |
| 7931 | acetyl | H | acetyl | acetyl | H | H | acetyl | acetyl | H | H | acetyl | acetyl |
| 7932 | acetyl | H | acetyl | acetyl | H | H | acetyl | acetyl | H | acetyl | H | H |
| 7933 | acetyl | H | acetyl | acetyl | H | H | acetyl | acetyl | H | acetyl | H | acetyl |
| 7934 | acetyl | H | acetyl | acetyl | H | H | acetyl | acetyl | H | acetyl | acetyl | H |
| 7935 | acetyl | H | acetyl | acetyl | H | H | acetyl | acetyl | H | acetyl | acetyl | acetyl |
| 7936 | acetyl | H | acetyl | acetyl | H | H | acetyl | acetyl | acetyl | H | H | H |
| 7937 | acetyl | H | acetyl | acetyl | H | H | acetyl | acetyl | acetyl | H | H | acetyl |
| 7938 | acetyl | H | acetyl | acetyl | H | H | acetyl | acetyl | acetyl | H | acetyl | H |
| 7939 | acetyl | H | acetyl | acetyl | H | H | acetyl | acetyl | acetyl | H | acetyl | acetyl |
| 7940 | acetyl | H | acetyl | acetyl | H | H | acetyl | acetyl | acetyl | acetyl | H | H |
| 7941 | acetyl | H | acetyl | acetyl | H | H | acetyl | acetyl | acetyl | acetyl | H | acetyl |
| 7942 | acetyl | H | acetyl | acetyl | H | H | acetyl | acetyl | acetyl | acetyl | acetyl | H |
| 7943 | acetyl | H | acetyl | acetyl | H | H | acetyl | acetyl | acetyl | acetyl | acetyl | acetyl |
| 7944 | acetyl | H | acetyl | acetyl | H | acetyl | H | H | H | H | H | H |
| 7945 | acetyl | H | acetyl | acetyl | H | acetyl | H | H | H | H | H | acetyl |
| 7946 | acetyl | H | acetyl | acetyl | H | acetyl | H | H | H | H | acetyl | H |
| 7947 | acetyl | H | acetyl | acetyl | H | acetyl | H | H | H | H | acetyl | acetyl |
| 7948 | acetyl | H | acetyl | acetyl | H | acetyl | H | H | H | acetyl | H | H |
| 7949 | acetyl | H | acetyl | acetyl | H | acetyl | H | H | H | acetyl | H | acetyl |
| 7950 | acetyl | H | acetyl | acetyl | H | acetyl | H | H | H | acetyl | acetyl | H |
| 7951 | acetyl | H | acetyl | acetyl | H | acetyl | H | H | H | acetyl | acetyl | acetyl |
| 7952 | acetyl | H | acetyl | acetyl | H | acetyl | H | H | acetyl | H | H | H |
| 7953 | acetyl | H | acetyl | acetyl | H | acetyl | H | H | acetyl | H | H | acetyl |
| 7954 | acetyl | H | acetyl | acetyl | H | acetyl | H | H | acetyl | H | acetyl | H |
| 7955 | acetyl | H | acetyl | acetyl | H | acetyl | H | H | acetyl | H | acetyl | acetyl |
| 7956 | acetyl | H | acetyl | acetyl | H | acetyl | H | H | acetyl | acetyl | H | H |
| 7957 | acetyl | H | acetyl | acetyl | H | acetyl | H | H | acetyl | acetyl | H | acetyl |
| 7958 | acetyl | H | acetyl | acetyl | H | acetyl | H | H | acetyl | acetyl | acetyl | H |
| 7959 | acetyl | H | acetyl | acetyl | H | acetyl | H | H | acetyl | acetyl | acetyl | acetyl |
| 7960 | acetyl | H | acetyl | acetyl | H | acetyl | H | acetyl | H | H | H | H |
| 7961 | acetyl | H | acetyl | acetyl | H | acetyl | H | acetyl | H | H | H | acetyl |
| 7962 | acetyl | H | acetyl | acetyl | H | acetyl | H | acetyl | H | H | acetyl | H |
| 7963 | acetyl | H | acetyl | acetyl | H | acetyl | H | acetyl | H | H | acetyl | acetyl |
| 7964 | acetyl | H | acetyl | acetyl | H | acetyl | H | acetyl | H | acetyl | H | H |
| 7965 | acetyl | H | acetyl | acetyl | H | acetyl | H | acetyl | H | acetyl | H | acetyl |
| 7966 | acetyl | H | acetyl | acetyl | H | acetyl | H | acetyl | H | acetyl | acetyl | H |
| 7967 | acetyl | H | acetyl | acetyl | H | acetyl | H | acetyl | H | acetyl | acetyl | acetyl |
| 7968 | acetyl | H | acetyl | acetyl | H | acetyl | H | acetyl | acetyl | H | H | H |
| 7969 | acetyl | H | acetyl | acetyl | H | acetyl | H | acetyl | acetyl | H | H | acetyl |
| 7970 | acetyl | H | acetyl | acetyl | H | acetyl | H | acetyl | acetyl | H | acetyl | H |
| 7971 | acetyl | H | acetyl | acetyl | H | acetyl | H | acetyl | acetyl | H | acetyl | acetyl |
| 7972 | acetyl | H | acetyl | acetyl | H | acetyl | H | acetyl | acetyl | acetyl | H | H |
| 7973 | acetyl | H | acetyl | acetyl | H | acetyl | H | acetyl | acetyl | acetyl | H | acetyl |
| 7974 | acetyl | H | acetyl | acetyl | H | acetyl | H | acetyl | acetyl | acetyl | acetyl | H |
| 7975 | acetyl | H | acetyl | acetyl | H | acetyl | H | acetyl | acetyl | acetyl | acetyl | acetyl |
| 7976 | acetyl | H | acetyl | acetyl | H | acetyl | acetyl | H | H | H | H | H |
| 7977 | acetyl | H | acetyl | acetyl | H | acetyl | acetyl | H | H | H | H | acetyl |
| 7978 | acetyl | H | acetyl | acetyl | H | acetyl | acetyl | H | H | H | acetyl | H |
| 7979 | acetyl | H | acetyl | acetyl | H | acetyl | acetyl | H | H | H | acetyl | acetyl |
| 7980 | acetyl | H | acetyl | acetyl | H | acetyl | acetyl | H | H | acetyl | H | H |
| 7981 | acetyl | H | acetyl | acetyl | H | acetyl | acetyl | H | H | acetyl | H | acetyl |
| 7982 | acetyl | H | acetyl | acetyl | H | acetyl | acetyl | H | H | acetyl | acetyl | H |
| 7983 | acetyl | H | acetyl | acetyl | H | acetyl | acetyl | H | H | acetyl | acetyl | acetyl |
| 7984 | acetyl | H | acetyl | acetyl | H | acetyl | acetyl | H | acetyl | H | H | H |
| 7985 | acetyl | H | acetyl | acetyl | H | acetyl | acetyl | H | acetyl | H | H | acetyl |
| 7986 | acetyl | H | acetyl | acetyl | H | acetyl | acetyl | H | acetyl | H | acetyl | H |
| 7987 | acetyl | H | acetyl | acetyl | H | acetyl | acetyl | H | acetyl | H | acetyl | acetyl |
| 7988 | acetyl | H | acetyl | acetyl | H | acetyl | acetyl | H | acetyl | acetyl | H | H |
| 7989 | acetyl | H | acetyl | acetyl | H | acetyl | acetyl | H | acetyl | acetyl | H | acetyl |
| 7990 | acetyl | H | acetyl | acetyl | H | acetyl | acetyl | H | acetyl | acetyl | acetyl | H |
| 7991 | acetyl | H | acetyl | acetyl | H | acetyl | acetyl | H | acetyl | acetyl | acetyl | acetyl |
| 7992 | acetyl | H | acetyl | acetyl | H | acetyl | acetyl | acetyl | H | H | H | H |
| 7993 | acetyl | H | acetyl | acetyl | H | acetyl | acetyl | acetyl | H | H | H | acetyl |
| 7994 | acetyl | H | acetyl | acetyl | H | acetyl | acetyl | acetyl | H | H | acetyl | H |
| 7995 | acetyl | H | acetyl | acetyl | H | acetyl | acetyl | acetyl | H | H | acetyl | acetyl |
| 7996 | acetyl | H | acetyl | acetyl | H | acetyl | acetyl | acetyl | H | acetyl | H | H |
| 7997 | acetyl | H | acetyl | acetyl | H | acetyl | acetyl | acetyl | H | acetyl | H | acetyl |
| 7998 | acetyl | H | acetyl | acetyl | H | acetyl | acetyl | acetyl | H | acetyl | acetyl | H |
| 7999 | acetyl | H | acetyl | acetyl | H | acetyl | acetyl | acetyl | H | acetyl | acetyl | acetyl |
| 8000 | acetyl | H | acetyl | acetyl | H | acetyl | acetyl | acetyl | acetyl | H | H | H |
| 8001 | acetyl | H | acetyl | acetyl | H | acetyl | acetyl | acetyl | acetyl | H | H | acetyl |
| 8002 | acetyl | H | acetyl | acetyl | H | acetyl | acetyl | acetyl | acetyl | H | acetyl | H |
| 8003 | acetyl | H | acetyl | acetyl | H | acetyl | acetyl | acetyl | acetyl | H | acetyl | acetyl |
| 8004 | acetyl | H | acetyl | acetyl | H | acetyl | acetyl | acetyl | acetyl | acetyl | H | H |
| 8005 | acetyl | H | acetyl | acetyl | H | acetyl | acetyl | acetyl | acetyl | acetyl | H | acetyl |
| 8006 | acetyl | H | acetyl | acetyl | H | acetyl | acetyl | acetyl | acetyl | acetyl | acetyl | H |
| 8007 | acetyl | H | acetyl | acetyl | H | acetyl | acetyl | acetyl | acetyl | acetyl | acetyl | acetyl |
| 8008 | acetyl | H | acetyl | acetyl | acetyl | H | H | H | H | H | H | H |
| 8009 | acetyl | H | acetyl | acetyl | acetyl | H | H | H | H | H | H | acetyl |
| 8010 | acetyl | H | acetyl | acetyl | acetyl | H | H | H | H | H | acetyl | H |
| 8011 | acetyl | H | acetyl | acetyl | acetyl | H | H | H | H | H | acetyl | acetyl |
| 8012 | acetyl | H | acetyl | acetyl | acetyl | H | H | H | H | acetyl | H | H |
| 8013 | acetyl | H | acetyl | acetyl | acetyl | H | H | H | H | acetyl | H | acetyl |
| 8014 | acetyl | H | acetyl | acetyl | acetyl | H | H | H | H | acetyl | acetyl | H |
| 8015 | acetyl | H | acetyl | acetyl | acetyl | H | H | H | H | acetyl | acetyl | acetyl |
| 8016 | acetyl | H | acetyl | acetyl | acetyl | H | H | H | acetyl | H | H | H |
| 8017 | acetyl | H | acetyl | acetyl | acetyl | H | H | H | acetyl | H | H | acetyl |
| 8018 | acetyl | H | acetyl | acetyl | acetyl | H | H | H | acetyl | H | acetyl | H |
| 8019 | acetyl | H | acetyl | acetyl | acetyl | H | H | H | acetyl | H | acetyl | acetyl |
| 8020 | acetyl | H | acetyl | acetyl | acetyl | H | H | H | acetyl | acetyl | H | H |
| 8021 | acetyl | H | acetyl | acetyl | acetyl | H | H | H | acetyl | acetyl | H | acetyl |
| 8022 | acetyl | H | acetyl | acetyl | acetyl | H | H | H | acetyl | acetyl | acetyl | H |
| 8023 | acetyl | H | acetyl | acetyl | acetyl | H | H | H | acetyl | acetyl | acetyl | acetyl |
| 8024 | acetyl | H | acetyl | acetyl | acetyl | H | H | acetyl | H | H | H | H |
| 8025 | acetyl | H | acetyl | acetyl | acetyl | H | H | acetyl | H | H | H | acetyl |
| 8026 | acetyl | H | acetyl | acetyl | acetyl | H | H | acetyl | H | H | acetyl | H |
| 8027 | acetyl | H | acetyl | acetyl | acetyl | H | H | acetyl | H | H | acetyl | acetyl |
| 8028 | acetyl | H | acetyl | acetyl | acetyl | H | H | acetyl | H | acetyl | H | H |
| 8029 | acetyl | H | acetyl | acetyl | acetyl | H | H | acetyl | H | acetyl | H | acetyl |
| 8030 | acetyl | H | acetyl | acetyl | acetyl | H | H | acetyl | H | acetyl | acetyl | H |
| 8031 | acetyl | H | acetyl | acetyl | acetyl | H | H | acetyl | H | acetyl | acetyl | acetyl |
| 8032 | acetyl | H | acetyl | acetyl | acetyl | H | H | acetyl | acetyl | H | H | H |
| 8033 | acetyl | H | acetyl | acetyl | acetyl | H | H | acetyl | acetyl | H | H | acetyl |
| 8034 | acetyl | H | acetyl | acetyl | acetyl | H | H | acetyl | acetyl | H | acetyl | H |
| 8035 | acetyl | H | acetyl | acetyl | acetyl | H | H | acetyl | acetyl | H | acetyl | acetyl |
| 8036 | acetyl | H | acetyl | acetyl | acetyl | H | H | acetyl | acetyl | acetyl | H | H |
| 8037 | acetyl | H | acetyl | acetyl | acetyl | H | H | acetyl | acetyl | acetyl | H | acetyl |
| 8038 | acetyl | H | acetyl | acetyl | acetyl | H | H | acetyl | acetyl | acetyl | acetyl | H |
| 8039 | acetyl | H | acetyl | acetyl | acetyl | H | H | acetyl | acetyl | acetyl | acetyl | acetyl |
| 8040 | acetyl | H | acetyl | acetyl | acetyl | H | acetyl | H | H | H | H | H |
| 8041 | acetyl | H | acetyl | acetyl | acetyl | H | acetyl | H | H | H | H | acetyl |
| 8042 | acetyl | H | acetyl | acetyl | acetyl | H | acetyl | H | H | H | acetyl | H |
| 8043 | acetyl | H | acetyl | acetyl | acetyl | H | acetyl | H | H | H | acetyl | acetyl |
| 8044 | acetyl | H | acetyl | acetyl | acetyl | H | acetyl | H | H | acetyl | H | H |
| 8045 | acetyl | H | acetyl | acetyl | acetyl | H | acetyl | H | H | acetyl | H | acetyl |
| 8046 | acetyl | H | acetyl | acetyl | acetyl | H | acetyl | H | H | acetyl | acetyl | H |
| 8047 | acetyl | H | acetyl | acetyl | acetyl | H | acetyl | H | H | acetyl | acetyl | acetyl |
| 8048 | acetyl | H | acetyl | acetyl | acetyl | H | acetyl | H | acetyl | H | H | H |
| 8049 | acetyl | H | acetyl | acetyl | acetyl | H | acetyl | H | acetyl | H | H | acetyl |
| 8050 | acetyl | H | acetyl | acetyl | acetyl | H | acetyl | H | acetyl | H | acetyl | H |
| 8051 | acetyl | H | acetyl | acetyl | acetyl | H | acetyl | H | acetyl | H | acetyl | acetyl |
| 8052 | acetyl | H | acetyl | acetyl | acetyl | H | acetyl | H | acetyl | acetyl | H | H |
| 8053 | acetyl | H | acetyl | acetyl | acetyl | H | acetyl | H | acetyl | acetyl | H | acetyl |
| 8054 | acetyl | H | acetyl | acetyl | acetyl | H | acetyl | H | acetyl | acetyl | acetyl | H |
| 8055 | acetyl | H | acetyl | acetyl | acetyl | H | acetyl | H | acetyl | acetyl | acetyl | acetyl |
| 8056 | acetyl | H | acetyl | acetyl | acetyl | H | acetyl | acetyl | H | H | H | H |
| 8057 | acetyl | H | acetyl | acetyl | acetyl | H | acetyl | acetyl | H | H | H | acetyl |
| 8058 | acetyl | H | acetyl | acetyl | acetyl | H | acetyl | acetyl | H | H | acetyl | H |
| 8059 | acetyl | H | acetyl | acetyl | acetyl | H | acetyl | acetyl | H | H | acetyl | acetyl |
| 8060 | acetyl | H | acetyl | acetyl | acetyl | H | acetyl | acetyl | H | acetyl | H | H |
| 8061 | acetyl | H | acetyl | acetyl | acetyl | H | acetyl | acetyl | H | acetyl | H | acetyl |
| 8062 | acetyl | H | acetyl | acetyl | acetyl | H | acetyl | acetyl | H | acetyl | acetyl | H |
| 8063 | acetyl | H | acetyl | acetyl | acetyl | H | acetyl | acetyl | H | acetyl | acetyl | acetyl |
| 8064 | acetyl | H | acetyl | acetyl | acetyl | H | acetyl | acetyl | acetyl | H | H | H |
| 8065 | acetyl | H | acetyl | acetyl | acetyl | H | acetyl | acetyl | acetyl | H | H | acetyl |
| 8066 | acetyl | H | acetyl | acetyl | acetyl | H | acetyl | acetyl | acetyl | H | acetyl | H |
| 8067 | acetyl | H | acetyl | acetyl | acetyl | H | acetyl | acetyl | acetyl | H | acetyl | acetyl |
| 8068 | acetyl | H | acetyl | acetyl | acetyl | H | acetyl | acetyl | acetyl | acetyl | H | H |
| 8069 | acetyl | H | acetyl | acetyl | acetyl | H | acetyl | acetyl | acetyl | acetyl | H | acetyl |
| 8070 | acetyl | H | acetyl | acetyl | acetyl | H | acetyl | acetyl | acetyl | acetyl | acetyl | H |
| 8071 | acetyl | H | acetyl | acetyl | acetyl | H | acetyl | acetyl | acetyl | acetyl | acetyl | acetyl |
| 8072 | acetyl | H | acetyl | acetyl | acetyl | acetyl | H | H | H | H | H | H |
| 8073 | acetyl | H | acetyl | acetyl | acetyl | acetyl | H | H | H | H | H | acetyl |
| 8074 | acetyl | H | acetyl | acetyl | acetyl | acetyl | H | H | H | H | acetyl | H |
| 8075 | acetyl | H | acetyl | acetyl | acetyl | acetyl | H | H | H | H | acetyl | acetyl |
| 8076 | acetyl | H | acetyl | acetyl | acetyl | acetyl | H | H | H | acetyl | H | H |
| 8077 | acetyl | H | acetyl | acetyl | acetyl | acetyl | H | H | H | acetyl | H | acetyl |
| 8078 | acetyl | H | acetyl | acetyl | acetyl | acetyl | H | H | H | acetyl | acetyl | H |
| 8079 | acetyl | H | acetyl | acetyl | acetyl | acetyl | H | H | H | acetyl | acetyl | acetyl |
| 8080 | acetyl | H | acetyl | acetyl | acetyl | acetyl | H | H | acetyl | H | H | H |
| 8081 | acetyl | H | acetyl | acetyl | acetyl | acetyl | H | H | acetyl | H | H | acetyl |
| 8082 | acetyl | H | acetyl | acetyl | acetyl | acetyl | H | H | acetyl | H | acetyl | H |
| 8083 | acetyl | H | acetyl | acetyl | acetyl | acetyl | H | H | acetyl | H | acetyl | acetyl |
| 8084 | acetyl | H | acetyl | acetyl | acetyl | acetyl | H | H | acetyl | acetyl | H | H |
| 8085 | acetyl | H | acetyl | acetyl | acetyl | acetyl | H | H | acetyl | acetyl | H | acetyl |
| 8086 | acetyl | H | acetyl | acetyl | acetyl | acetyl | H | H | acetyl | acetyl | acetyl | H |
| 8087 | acetyl | H | acetyl | acetyl | acetyl | acetyl | H | H | acetyl | acetyl | acetyl | acetyl |
| 8088 | acetyl | H | acetyl | acetyl | acetyl | acetyl | H | acetyl | H | H | H | H |
| 8089 | acetyl | H | acetyl | acetyl | acetyl | acetyl | H | acetyl | H | H | H | acetyl |
| 8090 | acetyl | H | acetyl | acetyl | acetyl | acetyl | H | acetyl | H | H | acetyl | H |
| 8091 | acetyl | H | acetyl | acetyl | acetyl | acetyl | H | acetyl | H | H | acetyl | acetyl |
| 8092 | acetyl | H | acetyl | acetyl | acetyl | acetyl | H | acetyl | H | acetyl | H | H |
| 8093 | acetyl | H | acetyl | acetyl | acetyl | acetyl | H | acetyl | H | acetyl | H | acetyl |
| 8094 | acetyl | H | acetyl | acetyl | acetyl | acetyl | H | acetyl | H | acetyl | acetyl | H |
| 8095 | acetyl | H | acetyl | acetyl | acetyl | acetyl | H | acetyl | H | acetyl | acetyl | acetyl |
| 8096 | acetyl | H | acetyl | acetyl | acetyl | acetyl | H | acetyl | acetyl | H | H | H |
| 8097 | acetyl | H | acetyl | acetyl | acetyl | acetyl | H | acetyl | acetyl | H | H | acetyl |
| 8098 | acetyl | H | acetyl | acetyl | acetyl | acetyl | H | acetyl | acetyl | H | acetyl | H |
| 8099 | acetyl | H | acetyl | acetyl | acetyl | acetyl | H | acetyl | acetyl | H | acetyl | acetyl |
| 8100 | acetyl | H | acetyl | acetyl | acetyl | acetyl | H | acetyl | acetyl | acetyl | H | H |
| 8101 | acetyl | H | acetyl | acetyl | acetyl | acetyl | H | acetyl | acetyl | acetyl | H | acetyl |
| 8102 | acetyl | H | acetyl | acetyl | acetyl | acetyl | H | acetyl | acetyl | acetyl | acetyl | H |
| 8103 | acetyl | H | acetyl | acetyl | acetyl | acetyl | H | acetyl | acetyl | acetyl | acetyl | acetyl |
| 8104 | acetyl | H | acetyl | acetyl | acetyl | acetyl | acetyl | H | H | H | H | H |
| 8105 | acetyl | H | acetyl | acetyl | acetyl | acetyl | acetyl | H | H | H | H | acetyl |
| 8106 | acetyl | H | acetyl | acetyl | acetyl | acetyl | acetyl | H | H | H | acetyl | H |
| 8107 | acetyl | H | acetyl | acetyl | acetyl | acetyl | acetyl | H | H | H | acetyl | acetyl |
| 8108 | acetyl | H | acetyl | acetyl | acetyl | acetyl | acetyl | H | H | acetyl | H | H |
| 8109 | acetyl | H | acetyl | acetyl | acetyl | acetyl | acetyl | H | H | acetyl | H | acetyl |
| 8110 | acetyl | H | acetyl | acetyl | acetyl | acetyl | acetyl | H | H | acetyl | acetyl | H |
| 8111 | acetyl | H | acetyl | acetyl | acetyl | acetyl | acetyl | H | H | acetyl | acetyl | acetyl |
| 8112 | acetyl | H | acetyl | acetyl | acetyl | acetyl | acetyl | H | acetyl | H | H | H |
| 8113 | acetyl | H | acetyl | acetyl | acetyl | acetyl | acetyl | H | acetyl | H | H | acetyl |
| 8114 | acetyl | H | acetyl | acetyl | acetyl | acetyl | acetyl | H | acetyl | H | acetyl | H |
| 8115 | acetyl | H | acetyl | acetyl | acetyl | acetyl | acetyl | H | acetyl | H | acetyl | acetyl |
| 8116 | acetyl | H | acetyl | acetyl | acetyl | acetyl | acetyl | H | acetyl | acetyl | H | H |
| 8117 | acetyl | H | acetyl | acetyl | acetyl | acetyl | acetyl | H | acetyl | acetyl | H | acetyl |
| 8118 | acetyl | H | acetyl | acetyl | acetyl | acetyl | acetyl | H | acetyl | acetyl | acetyl | H |
| 8119 | acetyl | H | acetyl | acetyl | acetyl | acetyl | acetyl | H | acetyl | acetyl | acetyl | acetyl |
| 8120 | acetyl | H | acetyl | acetyl | acetyl | acetyl | acetyl | acetyl | H | H | H | H |
| 8121 | acetyl | H | acetyl | acetyl | acetyl | acetyl | acetyl | acetyl | H | H | H | acetyl |
| 8122 | acetyl | H | acetyl | acetyl | acetyl | acetyl | acetyl | acetyl | H | H | acetyl | H |
| 8123 | acetyl | H | acetyl | acetyl | acetyl | acetyl | acetyl | acetyl | H | H | acetyl | acetyl |
| 8124 | acetyl | H | acetyl | acetyl | acetyl | acetyl | acetyl | acetyl | H | acetyl | H | H |
| 8125 | acetyl | H | acetyl | acetyl | acetyl | acetyl | acetyl | acetyl | H | acetyl | H | acetyl |
| 8126 | acetyl | H | acetyl | acetyl | acetyl | acetyl | acetyl | acetyl | H | acetyl | acetyl | H |
| 8127 | acetyl | H | acetyl | acetyl | acetyl | acetyl | acetyl | acetyl | H | acetyl | acetyl | acetyl |
| 8128 | acetyl | H | acetyl | acetyl | acetyl | acetyl | acetyl | acetyl | acetyl | H | H | H |
| 8129 | acetyl | H | acetyl | acetyl | acetyl | acetyl | acetyl | acetyl | acetyl | H | H | acetyl |
| 8130 | acetyl | H | acetyl | acetyl | acetyl | acetyl | acetyl | acetyl | acetyl | H | acetyl | H |
| 8131 | acetyl | H | acetyl | acetyl | acetyl | acetyl | acetyl | acetyl | acetyl | H | acetyl | acetyl |
| 8132 | acetyl | H | acetyl | acetyl | acetyl | acetyl | acetyl | acetyl | acetyl | acetyl | H | H |
| 8133 | acetyl | H | acetyl | acetyl | acetyl | acetyl | acetyl | acetyl | acetyl | acetyl | H | acetyl |
| 8134 | acetyl | H | acetyl | acetyl | acetyl | acetyl | acetyl | acetyl | acetyl | acetyl | acetyl | H |
| 8135 | acetyl | H | acetyl | acetyl | acetyl | acetyl | acetyl | acetyl | acetyl | acetyl | acetyl | acetyl |
| 8136 | acetyl | acetyl | H | H | H | H | H | H | H | H | H | H |
| 8137 | acetyl | acetyl | H | H | H | H | H | H | H | H | H | acetyl |
| 8138 | acetyl | acetyl | H | H | H | H | H | H | H | H | acetyl | H |
| 8139 | acetyl | acetyl | H | H | H | H | H | H | H | H | acetyl | acetyl |
| 8140 | acetyl | acetyl | H | H | H | H | H | H | H | acetyl | H | H |
| 8141 | acetyl | acetyl | H | H | H | H | H | H | H | acetyl | H | acetyl |
| 8142 | acetyl | acetyl | H | H | H | H | H | H | H | acetyl | acetyl | H |
| 8143 | acetyl | acetyl | H | H | H | H | H | H | H | acetyl | acetyl | acetyl |
| 8144 | acetyl | acetyl | H | H | H | H | H | H | acetyl | H | H | H |
| 8145 | acetyl | acetyl | H | H | H | H | H | H | acetyl | H | H | acetyl |
| 8146 | acetyl | acetyl | H | H | H | H | H | H | acetyl | H | acetyl | H |
| 8147 | acetyl | acetyl | H | H | H | H | H | H | acetyl | H | acetyl | acetyl |
| 8148 | acetyl | acetyl | H | H | H | H | H | H | acetyl | acetyl | H | H |
| 8149 | acetyl | acetyl | H | H | H | H | H | H | acetyl | acetyl | H | acetyl |
| 8150 | acetyl | acetyl | H | H | H | H | H | H | acetyl | acetyl | acetyl | H |
| 8151 | acetyl | acetyl | H | H | H | H | H | H | acetyl | acetyl | acetyl | acetyl |
| 8152 | acetyl | acetyl | H | H | H | H | H | acetyl | H | H | H | H |
| 8153 | acetyl | acetyl | H | H | H | H | H | acetyl | H | H | H | acetyl |
| 8154 | acetyl | acetyl | H | H | H | H | H | acetyl | H | H | acetyl | H |
| 8155 | acetyl | acetyl | H | H | H | H | H | acetyl | H | H | acetyl | acetyl |
| 8156 | acetyl | acetyl | H | H | H | H | H | acetyl | H | acetyl | H | H |
| 8157 | acetyl | acetyl | H | H | H | H | H | acetyl | H | acetyl | H | acetyl |
| 8158 | acetyl | acetyl | H | H | H | H | H | acetyl | H | acetyl | acetyl | H |
| 8159 | acetyl | acetyl | H | H | H | H | H | acetyl | H | acetyl | acetyl | acetyl |
| 8160 | acetyl | acetyl | H | H | H | H | H | acetyl | acetyl | H | H | H |
| 8161 | acetyl | acetyl | H | H | H | H | H | acetyl | acetyl | H | H | acetyl |
| 8162 | acetyl | acetyl | H | H | H | H | H | acetyl | acetyl | H | acetyl | H |
| 8163 | acetyl | acetyl | H | H | H | H | H | acetyl | acetyl | H | acetyl | acetyl |
| 8164 | acetyl | acetyl | H | H | H | H | H | acetyl | acetyl | acetyl | H | H |
| 8165 | acetyl | acetyl | H | H | H | H | H | acetyl | acetyl | acetyl | H | acetyl |
| 8166 | acetyl | acetyl | H | H | H | H | H | acetyl | acetyl | acetyl | acetyl | H |
| 8167 | acetyl | acetyl | H | H | H | H | H | acetyl | acetyl | acetyl | acetyl | acetyl |
| 8168 | acetyl | acetyl | H | H | H | H | acetyl | H | H | H | H | H |
| 8169 | acetyl | acetyl | H | H | H | H | acetyl | H | H | H | H | acetyl |
| 8170 | acetyl | acetyl | H | H | H | H | acetyl | H | H | H | acetyl | H |
| 8171 | acetyl | acetyl | H | H | H | H | acetyl | H | H | H | acetyl | acetyl |
| 8172 | acetyl | acetyl | H | H | H | H | acetyl | H | H | acetyl | H | H |
| 8173 | acetyl | acetyl | H | H | H | H | acetyl | H | H | acetyl | H | acetyl |
| 8174 | acetyl | acetyl | H | H | H | H | acetyl | H | H | acetyl | acetyl | H |
| 8175 | acetyl | acetyl | H | H | H | H | acetyl | H | H | acetyl | acetyl | acetyl |
| 8176 | acetyl | acetyl | H | H | H | H | acetyl | H | acetyl | H | H | H |
| 8177 | acetyl | acetyl | H | H | H | H | acetyl | H | acetyl | H | H | acetyl |
| 8178 | acetyl | acetyl | H | H | H | H | acetyl | H | acetyl | H | acetyl | H |
| 8179 | acetyl | acetyl | H | H | H | H | acetyl | H | acetyl | H | acetyl | acetyl |
| 8180 | acetyl | acetyl | H | H | H | H | acetyl | H | acetyl | acetyl | H | H |
| 8181 | acetyl | acetyl | H | H | H | H | acetyl | H | acetyl | acetyl | H | acetyl |
| 8182 | acetyl | acetyl | H | H | H | H | acetyl | H | acetyl | acetyl | acetyl | H |
| 8183 | acetyl | acetyl | H | H | H | H | acetyl | H | acetyl | acetyl | acetyl | acetyl |
| 8184 | acetyl | acetyl | H | H | H | H | acetyl | acetyl | H | H | H | H |
| 8185 | acetyl | acetyl | H | H | H | H | acetyl | acetyl | H | H | H | acetyl |
| 8186 | acetyl | acetyl | H | H | H | H | acetyl | acetyl | H | H | acetyl | H |
| 8187 | acetyl | acetyl | H | H | H | H | acetyl | acetyl | H | H | acetyl | acetyl |
| 8188 | acetyl | acetyl | H | H | H | H | acetyl | acetyl | H | acetyl | H | H |
| 8189 | acetyl | acetyl | H | H | H | H | acetyl | acetyl | H | acetyl | H | acetyl |
| 8190 | acetyl | acetyl | H | H | H | H | acetyl | acetyl | H | acetyl | acetyl | H |
| 8191 | acetyl | acetyl | H | H | H | H | acetyl | acetyl | H | acetyl | acetyl | acetyl |
| 8192 | acetyl | acetyl | H | H | H | H | acetyl | acetyl | acetyl | H | H | H |
| 8193 | acetyl | acetyl | H | H | H | H | acetyl | acetyl | acetyl | H | H | acetyl |
| 8194 | acetyl | acetyl | H | H | H | H | acetyl | acetyl | acetyl | H | acetyl | H |
| 8195 | acetyl | acetyl | H | H | H | H | acetyl | acetyl | acetyl | H | acetyl | acetyl |
| 8196 | acetyl | acetyl | H | H | H | H | acetyl | acetyl | acetyl | acetyl | H | H |
| 8197 | acetyl | acetyl | H | H | H | H | acetyl | acetyl | acetyl | acetyl | H | acetyl |
| 8198 | acetyl | acetyl | H | H | H | H | acetyl | acetyl | acetyl | acetyl | acetyl | H |
| 8199 | acetyl | acetyl | H | H | H | H | acetyl | acetyl | acetyl | acetyl | acetyl | acetyl |
| 8200 | acetyl | acetyl | H | H | H | acetyl | H | H | H | H | H | H |
| 8201 | acetyl | acetyl | H | H | H | acetyl | H | H | H | H | H | acetyl |
| 8202 | acetyl | acetyl | H | H | H | acetyl | H | H | H | H | acetyl | H |
| 8203 | acetyl | acetyl | H | H | H | acetyl | H | H | H | H | acetyl | acetyl |
| 8204 | acetyl | acetyl | H | H | H | acetyl | H | H | H | acetyl | H | H |
| 8205 | acetyl | acetyl | H | H | H | acetyl | H | H | H | acetyl | H | acetyl |
| 8206 | acetyl | acetyl | H | H | H | acetyl | H | H | H | acetyl | acetyl | H |
| 8207 | acetyl | acetyl | H | H | H | acetyl | H | H | H | acetyl | acetyl | acetyl |
| 8208 | acetyl | acetyl | H | H | H | acetyl | H | H | acetyl | H | H | H |
| 8209 | acetyl | acetyl | H | H | H | acetyl | H | H | acetyl | H | H | acetyl |
| 8210 | acetyl | acetyl | H | H | H | acetyl | H | H | acetyl | H | acetyl | H |
| 8211 | acetyl | acetyl | H | H | H | acetyl | H | H | acetyl | H | acetyl | acetyl |
| 8212 | acetyl | acetyl | H | H | H | acetyl | H | H | acetyl | acetyl | H | H |
| 8213 | acetyl | acetyl | H | H | H | acetyl | H | H | acetyl | acetyl | H | acetyl |
| 8214 | acetyl | acetyl | H | H | H | acetyl | H | H | acetyl | acetyl | acetyl | H |
| 8215 | acetyl | acetyl | H | H | H | acetyl | H | H | acetyl | acetyl | acetyl | acetyl |
| 8216 | acetyl | acetyl | H | H | H | acetyl | H | acetyl | H | H | H | H |
| 8217 | acetyl | acetyl | H | H | H | acetyl | H | acetyl | H | H | H | acetyl |
| 8218 | acetyl | acetyl | H | H | H | acetyl | H | acetyl | H | H | acetyl | H |
| 8219 | acetyl | acetyl | H | H | H | acetyl | H | acetyl | H | H | acetyl | acetyl |
| 8220 | acetyl | acetyl | H | H | H | acetyl | H | acetyl | H | acetyl | H | H |
| 8221 | acetyl | acetyl | H | H | H | acetyl | H | acetyl | H | acetyl | H | acetyl |
| 8222 | acetyl | acetyl | H | H | H | acetyl | H | acetyl | H | acetyl | acetyl | H |
| 8223 | acetyl | acetyl | H | H | H | acetyl | H | acetyl | H | acetyl | acetyl | acetyl |
| 8224 | acetyl | acetyl | H | H | H | acetyl | H | acetyl | acetyl | H | H | H |
| 8225 | acetyl | acetyl | H | H | H | acetyl | H | acetyl | acetyl | H | H | acetyl |
| 8226 | acetyl | acetyl | H | H | H | acetyl | H | acetyl | acetyl | H | acetyl | H |
| 8227 | acetyl | acetyl | H | H | H | acetyl | H | acetyl | acetyl | H | acetyl | acetyl |
| 8228 | acetyl | acetyl | H | H | H | acetyl | H | acetyl | acetyl | acetyl | H | H |
| 8229 | acetyl | acetyl | H | H | H | acetyl | H | acetyl | acetyl | acetyl | H | acetyl |
| 8230 | acetyl | acetyl | H | H | H | acetyl | H | acetyl | acetyl | acetyl | acetyl | H |
| 8231 | acetyl | acetyl | H | H | H | acetyl | H | acetyl | acetyl | acetyl | acetyl | acetyl |
| 8232 | acetyl | acetyl | H | H | H | acetyl | acetyl | H | H | H | H | H |
| 8233 | acetyl | acetyl | H | H | H | acetyl | acetyl | H | H | H | H | acetyl |
| 8234 | acetyl | acetyl | H | H | H | acetyl | acetyl | H | H | H | acetyl | H |
| 8235 | acetyl | acetyl | H | H | H | acetyl | acetyl | H | H | H | acetyl | acetyl |
| 8236 | acetyl | acetyl | H | H | H | acetyl | acetyl | H | H | acetyl | H | H |
| 8237 | acetyl | acetyl | H | H | H | acetyl | acetyl | H | H | acetyl | H | acetyl |
| 8238 | acetyl | acetyl | H | H | H | acetyl | acetyl | H | H | acetyl | acetyl | H |
| 8239 | acetyl | acetyl | H | H | H | acetyl | acetyl | H | H | acetyl | acetyl | acetyl |
| 8240 | acetyl | acetyl | H | H | H | acetyl | acetyl | H | acetyl | H | H | H |
| 8241 | acetyl | acetyl | H | H | H | acetyl | acetyl | H | acetyl | H | H | acetyl |
| 8242 | acetyl | acetyl | H | H | H | acetyl | acetyl | H | acetyl | H | acetyl | H |
| 8243 | acetyl | acetyl | H | H | H | acetyl | acetyl | H | acetyl | H | acetyl | acetyl |
| 8244 | acetyl | acetyl | H | H | H | acetyl | acetyl | H | acetyl | acetyl | H | H |
| 8245 | acetyl | acetyl | H | H | H | acetyl | acetyl | H | acetyl | acetyl | H | acetyl |
| 8246 | acetyl | acetyl | H | H | H | acetyl | acetyl | H | acetyl | acetyl | acetyl | H |
| 8247 | acetyl | acetyl | H | H | H | acetyl | acetyl | H | acetyl | acetyl | acetyl | acetyl |
| 8248 | acetyl | acetyl | H | H | H | acetyl | acetyl | acetyl | H | H | H | H |
| 8249 | acetyl | acetyl | H | H | H | acetyl | acetyl | acetyl | H | H | H | acetyl |
| 8250 | acetyl | acetyl | H | H | H | acetyl | acetyl | acetyl | H | H | acetyl | H |
| 8251 | acetyl | acetyl | H | H | H | acetyl | acetyl | acetyl | H | H | acetyl | acetyl |
| 8252 | acetyl | acetyl | H | H | H | acetyl | acetyl | acetyl | H | acetyl | H | H |
| 8253 | acetyl | acetyl | H | H | H | acetyl | acetyl | acetyl | H | acetyl | H | acetyl |
| 8254 | acetyl | acetyl | H | H | H | acetyl | acetyl | acetyl | H | acetyl | acetyl | H |
| 8255 | acetyl | acetyl | H | H | H | acetyl | acetyl | acetyl | H | acetyl | acetyl | acetyl |
| 8256 | acetyl | acetyl | H | H | H | acetyl | acetyl | acetyl | acetyl | H | H | H |
| 8257 | acetyl | acetyl | H | H | H | acetyl | acetyl | acetyl | acetyl | H | H | acetyl |
| 8258 | acetyl | acetyl | H | H | H | acetyl | acetyl | acetyl | acetyl | H | acetyl | H |
| 8259 | acetyl | acetyl | H | H | H | acetyl | acetyl | acetyl | acetyl | H | acetyl | acetyl |
| 8260 | acetyl | acetyl | H | H | H | acetyl | acetyl | acetyl | acetyl | acetyl | H | H |
| 8261 | acetyl | acetyl | H | H | H | acetyl | acetyl | acetyl | acetyl | acetyl | H | acetyl |
| 8262 | acetyl | acetyl | H | H | H | acetyl | acetyl | acetyl | acetyl | acetyl | acetyl | H |
| 8263 | acetyl | acetyl | H | H | H | acetyl | acetyl | acetyl | acetyl | acetyl | acetyl | acetyl |
| 8264 | acetyl | acetyl | H | H | acetyl | H | H | H | H | H | H | H |
| 8265 | acetyl | acetyl | H | H | acetyl | H | H | H | H | H | H | acetyl |
| 8266 | acetyl | acetyl | H | H | acetyl | H | H | H | H | H | acetyl | H |
| 8267 | acetyl | acetyl | H | H | acetyl | H | H | H | H | H | acetyl | acetyl |
| 8268 | acetyl | acetyl | H | H | acetyl | H | H | H | H | acetyl | H | H |
| 8269 | acetyl | acetyl | H | H | acetyl | H | H | H | H | acetyl | H | acetyl |
| 8270 | acetyl | acetyl | H | H | acetyl | H | H | H | H | acetyl | acetyl | H |
| 8271 | acetyl | acetyl | H | H | acetyl | H | H | H | H | acetyl | acetyl | acetyl |
| 8272 | acetyl | acetyl | H | H | acetyl | H | H | H | acetyl | H | H | H |
| 8273 | acetyl | acetyl | H | H | acetyl | H | H | H | acetyl | H | H | acetyl |
| 8274 | acetyl | acetyl | H | H | acetyl | H | H | H | acetyl | H | acetyl | H |
| 8275 | acetyl | acetyl | H | H | acetyl | H | H | H | acetyl | H | acetyl | acetyl |
| 8276 | acetyl | acetyl | H | H | acetyl | H | H | H | acetyl | acetyl | H | H |
| 8277 | acetyl | acetyl | H | H | acetyl | H | H | H | acetyl | acetyl | H | acetyl |
| 8278 | acetyl | acetyl | H | H | acetyl | H | H | H | acetyl | acetyl | acetyl | H |
| 8279 | acetyl | acetyl | H | H | acetyl | H | H | H | acetyl | acetyl | acetyl | acetyl |
| 8280 | acetyl | acetyl | H | H | acetyl | H | H | acetyl | H | H | H | H |
| 8281 | acetyl | acetyl | H | H | acetyl | H | H | acetyl | H | H | H | acetyl |
| 8282 | acetyl | acetyl | H | H | acetyl | H | H | acetyl | H | H | acetyl | H |
| 8283 | acetyl | acetyl | H | H | acetyl | H | H | acetyl | H | H | acetyl | acetyl |
| 8284 | acetyl | acetyl | H | H | acetyl | H | H | acetyl | H | acetyl | H | H |
| 8285 | acetyl | acetyl | H | H | acetyl | H | H | acetyl | H | acetyl | H | acetyl |
| 8286 | acetyl | acetyl | H | H | acetyl | H | H | acetyl | H | acetyl | acetyl | H |
| 8287 | acetyl | acetyl | H | H | acetyl | H | H | acetyl | H | acetyl | acetyl | acetyl |
| 8288 | acetyl | acetyl | H | H | acetyl | H | H | acetyl | acetyl | H | H | H |
| 8289 | acetyl | acetyl | H | H | acetyl | H | H | acetyl | acetyl | H | H | acetyl |
| 8290 | acetyl | acetyl | H | H | acetyl | H | H | acetyl | acetyl | H | acetyl | H |
| 8291 | acetyl | acetyl | H | H | acetyl | H | H | acetyl | acetyl | H | acetyl | acetyl |
| 8292 | acetyl | acetyl | H | H | acetyl | H | H | acetyl | acetyl | acetyl | H | H |
| 8293 | acetyl | acetyl | H | H | acetyl | H | H | acetyl | acetyl | acetyl | H | acetyl |
| 8294 | acetyl | acetyl | H | H | acetyl | H | H | acetyl | acetyl | acetyl | acetyl | H |
| 8295 | acetyl | acetyl | H | H | acetyl | H | H | acetyl | acetyl | acetyl | acetyl | acetyl |
| 8296 | acetyl | acetyl | H | H | acetyl | H | acetyl | H | H | H | H | H |
| 8297 | acetyl | acetyl | H | H | acetyl | H | acetyl | H | H | H | H | acetyl |
| 8298 | acetyl | acetyl | H | H | acetyl | H | acetyl | H | H | H | acetyl | H |
| 8299 | acetyl | acetyl | H | H | acetyl | H | acetyl | H | H | H | acetyl | acetyl |
| 8300 | acetyl | acetyl | H | H | acetyl | H | acetyl | H | H | acetyl | H | H |
| 8301 | acetyl | acetyl | H | H | acetyl | H | acetyl | H | H | acetyl | H | acetyl |
| 8302 | acetyl | acetyl | H | H | acetyl | H | acetyl | H | H | acetyl | acetyl | H |
| 8303 | acetyl | acetyl | H | H | acetyl | H | acetyl | H | H | acetyl | acetyl | acetyl |
| 8304 | acetyl | acetyl | H | H | acetyl | H | acetyl | H | acetyl | H | H | H |
| 8305 | acetyl | acetyl | H | H | acetyl | H | acetyl | H | acetyl | H | H | acetyl |
| 8306 | acetyl | acetyl | H | H | acetyl | H | acetyl | H | acetyl | H | acetyl | H |
| 8307 | acetyl | acetyl | H | H | acetyl | H | acetyl | H | acetyl | H | acetyl | acetyl |
| 8308 | acetyl | acetyl | H | H | acetyl | H | acetyl | H | acetyl | acetyl | H | H |
| 8309 | acetyl | acetyl | H | H | acetyl | H | acetyl | H | acetyl | acetyl | H | acetyl |
| 8310 | acetyl | acetyl | H | H | acetyl | H | acetyl | H | acetyl | acetyl | acetyl | H |
| 8311 | acetyl | acetyl | H | H | acetyl | H | acetyl | H | acetyl | acetyl | acetyl | acetyl |
| 8312 | acetyl | acetyl | H | H | acetyl | H | acetyl | acetyl | H | H | H | H |
| 8313 | acetyl | acetyl | H | H | acetyl | H | acetyl | acetyl | H | H | H | acetyl |
| 8314 | acetyl | acetyl | H | H | acetyl | H | acetyl | acetyl | H | H | acetyl | H |
| 8315 | acetyl | acetyl | H | H | acetyl | H | acetyl | acetyl | H | H | acetyl | acetyl |
| 8316 | acetyl | acetyl | H | H | acetyl | H | acetyl | acetyl | H | acetyl | H | H |
| 8317 | acetyl | acetyl | H | H | acetyl | H | acetyl | acetyl | H | acetyl | H | acetyl |
| 8318 | acetyl | acetyl | H | H | acetyl | H | acetyl | acetyl | H | acetyl | acetyl | H |
| 8319 | acetyl | acetyl | H | H | acetyl | H | acetyl | acetyl | H | acetyl | acetyl | acetyl |
| 8320 | acetyl | acetyl | H | H | acetyl | H | acetyl | acetyl | acetyl | H | H | H |
| 8321 | acetyl | acetyl | H | H | acetyl | H | acetyl | acetyl | acetyl | H | H | acetyl |
| 8322 | acetyl | acetyl | H | H | acetyl | H | acetyl | acetyl | acetyl | H | acetyl | H |
| 8323 | acetyl | acetyl | H | H | acetyl | H | acetyl | acetyl | acetyl | H | acetyl | acetyl |
| 8324 | acetyl | acetyl | H | H | acetyl | H | acetyl | acetyl | acetyl | acetyl | H | H |
| 8325 | acetyl | acetyl | H | H | acetyl | H | acetyl | acetyl | acetyl | acetyl | H | acetyl |
| 8326 | acetyl | acetyl | H | H | acetyl | H | acetyl | acetyl | acetyl | acetyl | acetyl | H |
| 8327 | acetyl | acetyl | H | H | acetyl | H | acetyl | acetyl | acetyl | acetyl | acetyl | acetyl |
| 8328 | acetyl | acetyl | H | H | acetyl | acetyl | H | H | H | H | H | H |
| 8329 | acetyl | acetyl | H | H | acetyl | acetyl | H | H | H | H | H | acetyl |
| 8330 | acetyl | acetyl | H | H | acetyl | acetyl | H | H | H | H | acetyl | H |
| 8331 | acetyl | acetyl | H | H | acetyl | acetyl | H | H | H | H | acetyl | acetyl |
| 8332 | acetyl | acetyl | H | H | acetyl | acetyl | H | H | H | acetyl | H | H |
| 8333 | acetyl | acetyl | H | H | acetyl | acetyl | H | H | H | acetyl | H | acetyl |
| 8334 | acetyl | acetyl | H | H | acetyl | acetyl | H | H | H | acetyl | acetyl | H |
| 8335 | acetyl | acetyl | H | H | acetyl | acetyl | H | H | H | acetyl | acetyl | acetyl |
| 8336 | acetyl | acetyl | H | H | acetyl | acetyl | H | H | acetyl | H | H | H |
| 8337 | acetyl | acetyl | H | H | acetyl | acetyl | H | H | acetyl | H | H | acetyl |
| 8338 | acetyl | acetyl | H | H | acetyl | acetyl | H | H | acetyl | H | acetyl | H |
| 8339 | acetyl | acetyl | H | H | acetyl | acetyl | H | H | acetyl | H | acetyl | acetyl |
| 8340 | acetyl | acetyl | H | H | acetyl | acetyl | H | H | acetyl | acetyl | H | H |
| 8341 | acetyl | acetyl | H | H | acetyl | acetyl | H | H | acetyl | acetyl | H | acetyl |
| 8342 | acetyl | acetyl | H | H | acetyl | acetyl | H | H | acetyl | acetyl | acetyl | H |
| 8343 | acetyl | acetyl | H | H | acetyl | acetyl | H | H | acetyl | acetyl | acetyl | acetyl |
| 8344 | acetyl | acetyl | H | H | acetyl | acetyl | H | acetyl | H | H | H | H |
| 8345 | acetyl | acetyl | H | H | acetyl | acetyl | H | acetyl | H | H | H | acetyl |
| 8346 | acetyl | acetyl | H | H | acetyl | acetyl | H | acetyl | H | H | acetyl | H |
| 8347 | acetyl | acetyl | H | H | acetyl | acetyl | H | acetyl | H | H | acetyl | acetyl |
| 8348 | acetyl | acetyl | H | H | acetyl | acetyl | H | acetyl | H | acetyl | H | H |
| 8349 | acetyl | acetyl | H | H | acetyl | acetyl | H | acetyl | H | acetyl | H | acetyl |
| 8350 | acetyl | acetyl | H | H | acetyl | acetyl | H | acetyl | H | acetyl | acetyl | H |
| 8351 | acetyl | acetyl | H | H | acetyl | acetyl | H | acetyl | H | acetyl | acetyl | acetyl |
| 8352 | acetyl | acetyl | H | H | acetyl | acetyl | H | acetyl | acetyl | H | H | H |
| 8353 | acetyl | acetyl | H | H | acetyl | acetyl | H | acetyl | acetyl | H | H | acetyl |
| 8354 | acetyl | acetyl | H | H | acetyl | acetyl | H | acetyl | acetyl | H | acetyl | H |
| 8355 | acetyl | acetyl | H | H | acetyl | acetyl | H | acetyl | acetyl | H | acetyl | acetyl |
| 8356 | acetyl | acetyl | H | H | acetyl | acetyl | H | acetyl | acetyl | acetyl | H | H |
| 8357 | acetyl | acetyl | H | H | acetyl | acetyl | H | acetyl | acetyl | acetyl | H | acetyl |
| 8358 | acetyl | acetyl | H | H | acetyl | acetyl | H | acetyl | acetyl | acetyl | acetyl | H |
| 8359 | acetyl | acetyl | H | H | acetyl | acetyl | H | acetyl | acetyl | acetyl | acetyl | acetyl |
| 8360 | acetyl | acetyl | H | H | acetyl | acetyl | acetyl | H | H | H | H | H |
| 8361 | acetyl | acetyl | H | H | acetyl | acetyl | acetyl | H | H | H | H | acetyl |
| 8362 | acetyl | acetyl | H | H | acetyl | acetyl | acetyl | H | H | H | acetyl | H |
| 8363 | acetyl | acetyl | H | H | acetyl | acetyl | acetyl | H | H | H | acetyl | acetyl |
| 8364 | acetyl | acetyl | H | H | acetyl | acetyl | acetyl | H | H | acetyl | H | H |
| 8365 | acetyl | acetyl | H | H | acetyl | acetyl | acetyl | H | H | acetyl | H | acetyl |
| 8366 | acetyl | acetyl | H | H | acetyl | acetyl | acetyl | H | H | acetyl | acetyl | H |
| 8367 | acetyl | acetyl | H | H | acetyl | acetyl | acetyl | H | H | acetyl | acetyl | acetyl |
| 8368 | acetyl | acetyl | H | H | acetyl | acetyl | acetyl | H | acetyl | H | H | H |
| 8369 | acetyl | acetyl | H | H | acetyl | acetyl | acetyl | H | acetyl | H | H | acetyl |
| 8370 | acetyl | acetyl | H | H | acetyl | acetyl | acetyl | H | acetyl | H | acetyl | H |
| 8371 | acetyl | acetyl | H | H | acetyl | acetyl | acetyl | H | acetyl | H | acetyl | acetyl |
| 8372 | acetyl | acetyl | H | H | acetyl | acetyl | acetyl | H | acetyl | acetyl | H | H |
| 8373 | acetyl | acetyl | H | H | acetyl | acetyl | acetyl | H | acetyl | acetyl | H | acetyl |
| 8374 | acetyl | acetyl | H | H | acetyl | acetyl | acetyl | H | acetyl | acetyl | acetyl | H |
| 8375 | acetyl | acetyl | H | H | acetyl | acetyl | acetyl | H | acetyl | acetyl | acetyl | acetyl |
| 8376 | acetyl | acetyl | H | H | acetyl | acetyl | acetyl | acetyl | H | H | H | H |
| 8377 | acetyl | acetyl | H | H | acetyl | acetyl | acetyl | acetyl | H | H | H | acetyl |
| 8378 | acetyl | acetyl | H | H | acetyl | acetyl | acetyl | acetyl | H | H | acetyl | H |
| 8379 | acetyl | acetyl | H | H | acetyl | acetyl | acetyl | acetyl | H | H | acetyl | acetyl |
| 8380 | acetyl | acetyl | H | H | acetyl | acetyl | acetyl | acetyl | H | acetyl | H | H |
| 8381 | acetyl | acetyl | H | H | acetyl | acetyl | acetyl | acetyl | H | acetyl | H | acetyl |
| 8382 | acetyl | acetyl | H | H | acetyl | acetyl | acetyl | acetyl | H | acetyl | acetyl | H |
| 8383 | acetyl | acetyl | H | H | acetyl | acetyl | acetyl | acetyl | H | acetyl | acetyl | acetyl |
| 8384 | acetyl | acetyl | H | H | acetyl | acetyl | acetyl | acetyl | acetyl | H | H | H |
| 8385 | acetyl | acetyl | H | H | acetyl | acetyl | acetyl | acetyl | acetyl | H | H | acetyl |
| 8386 | acetyl | acetyl | H | H | acetyl | acetyl | acetyl | acetyl | acetyl | H | acetyl | H |
| 8387 | acetyl | acetyl | H | H | acetyl | acetyl | acetyl | acetyl | acetyl | H | acetyl | acetyl |
| 8388 | acetyl | acetyl | H | H | acetyl | acetyl | acetyl | acetyl | acetyl | acetyl | H | H |
| 8389 | acetyl | acetyl | H | H | acetyl | acetyl | acetyl | acetyl | acetyl | acetyl | H | acetyl |
| 8390 | acetyl | acetyl | H | H | acetyl | acetyl | acetyl | acetyl | acetyl | acetyl | acetyl | H |
| 8391 | acetyl | acetyl | H | H | acetyl | acetyl | acetyl | acetyl | acetyl | acetyl | acetyl | acetyl |
| 8392 | acetyl | acetyl | H | acetyl | H | H | H | H | H | H | H | H |
| 8393 | acetyl | acetyl | H | acetyl | H | H | H | H | H | H | H | acetyl |
| 8394 | acetyl | acetyl | H | acetyl | H | H | H | H | H | H | acetyl | H |
| 8395 | acetyl | acetyl | H | acetyl | H | H | H | H | H | H | acetyl | acetyl |
| 8396 | acetyl | acetyl | H | acetyl | H | H | H | H | H | acetyl | H | H |
| 8397 | acetyl | acetyl | H | acetyl | H | H | H | H | H | acetyl | H | acetyl |
| 8398 | acetyl | acetyl | H | acetyl | H | H | H | H | H | acetyl | acetyl | H |
| 8399 | acetyl | acetyl | H | acetyl | H | H | H | H | H | acetyl | acetyl | acetyl |
| 8400 | acetyl | acetyl | H | acetyl | H | H | H | H | acetyl | H | H | H |
| 8401 | acetyl | acetyl | H | acetyl | H | H | H | H | acetyl | H | H | acetyl |
| 8402 | acetyl | acetyl | H | acetyl | H | H | H | H | acetyl | H | acetyl | H |
| 8403 | acetyl | acetyl | H | acetyl | H | H | H | H | acetyl | H | acetyl | acetyl |
| 8404 | acetyl | acetyl | H | acetyl | H | H | H | H | acetyl | acetyl | H | H |
| 8405 | acetyl | acetyl | H | acetyl | H | H | H | H | acetyl | acetyl | H | acetyl |
| 8406 | acetyl | acetyl | H | acetyl | H | H | H | H | acetyl | acetyl | acetyl | H |
| 8407 | acetyl | acetyl | H | acetyl | H | H | H | H | acetyl | acetyl | acetyl | acetyl |
| 8408 | acetyl | acetyl | H | acetyl | H | H | H | acetyl | H | H | H | H |
| 8409 | acetyl | acetyl | H | acetyl | H | H | H | acetyl | H | H | H | acetyl |
| 8410 | acetyl | acetyl | H | acetyl | H | H | H | acetyl | H | H | acetyl | H |
| 8411 | acetyl | acetyl | H | acetyl | H | H | H | acetyl | H | H | acetyl | acetyl |
| 8412 | acetyl | acetyl | H | acetyl | H | H | H | acetyl | H | acetyl | H | H |
| 8413 | acetyl | acetyl | H | acetyl | H | H | H | acetyl | H | acetyl | H | acetyl |
| 8414 | acetyl | acetyl | H | acetyl | H | H | H | acetyl | H | acetyl | acetyl | H |
| 8415 | acetyl | acetyl | H | acetyl | H | H | H | acetyl | H | acetyl | acetyl | acetyl |
| 8416 | acetyl | acetyl | H | acetyl | H | H | H | acetyl | acetyl | H | H | H |
| 8417 | acetyl | acetyl | H | acetyl | H | H | H | acetyl | acetyl | H | H | acetyl |
| 8418 | acetyl | acetyl | H | acetyl | H | H | H | acetyl | acetyl | H | acetyl | H |
| 8419 | acetyl | acetyl | H | acetyl | H | H | H | acetyl | acetyl | H | acetyl | acetyl |
| 8420 | acetyl | acetyl | H | acetyl | H | H | H | acetyl | acetyl | acetyl | H | H |
| 8421 | acetyl | acetyl | H | acetyl | H | H | H | acetyl | acetyl | acetyl | H | acetyl |
| 8422 | acetyl | acetyl | H | acetyl | H | H | H | acetyl | acetyl | acetyl | acetyl | H |
| 8423 | acetyl | acetyl | H | acetyl | H | H | H | acetyl | acetyl | acetyl | acetyl | acetyl |
| 8424 | acetyl | acetyl | H | acetyl | H | H | acetyl | H | H | H | H | H |
| 8425 | acetyl | acetyl | H | acetyl | H | H | acetyl | H | H | H | H | acetyl |
| 8426 | acetyl | acetyl | H | acetyl | H | H | acetyl | H | H | H | acetyl | H |
| 8427 | acetyl | acetyl | H | acetyl | H | H | acetyl | H | H | H | acetyl | acetyl |
| 8428 | acetyl | acetyl | H | acetyl | H | H | acetyl | H | H | acetyl | H | H |
| 8429 | acetyl | acetyl | H | acetyl | H | H | acetyl | H | H | acetyl | H | acetyl |
| 8430 | acetyl | acetyl | H | acetyl | H | H | acetyl | H | H | acetyl | acetyl | H |
| 8431 | acetyl | acetyl | H | acetyl | H | H | acetyl | H | H | acetyl | acetyl | acetyl |
| 8432 | acetyl | acetyl | H | acetyl | H | H | acetyl | H | acetyl | H | H | H |
| 8433 | acetyl | acetyl | H | acetyl | H | H | acetyl | H | acetyl | H | H | acetyl |
| 8434 | acetyl | acetyl | H | acetyl | H | H | acetyl | H | acetyl | H | acetyl | H |
| 8435 | acetyl | acetyl | H | acetyl | H | H | acetyl | H | acetyl | H | acetyl | acetyl |
| 8436 | acetyl | acetyl | H | acetyl | H | H | acetyl | H | acetyl | acetyl | H | H |
| 8437 | acetyl | acetyl | H | acetyl | H | H | acetyl | H | acetyl | acetyl | H | acetyl |
| 8438 | acetyl | acetyl | H | acetyl | H | H | acetyl | H | acetyl | acetyl | acetyl | H |
| 8439 | acetyl | acetyl | H | acetyl | H | H | acetyl | H | acetyl | acetyl | acetyl | acetyl |
| 8440 | acetyl | acetyl | H | acetyl | H | H | acetyl | acetyl | H | H | H | H |
| 8441 | acetyl | acetyl | H | acetyl | H | H | acetyl | acetyl | H | H | H | acetyl |
| 8442 | acetyl | acetyl | H | acetyl | H | H | acetyl | acetyl | H | H | acetyl | H |
| 8443 | acetyl | acetyl | H | acetyl | H | H | acetyl | acetyl | H | H | acetyl | acetyl |
| 8444 | acetyl | acetyl | H | acetyl | H | H | acetyl | acetyl | H | acetyl | H | H |
| 8445 | acetyl | acetyl | H | acetyl | H | H | acetyl | acetyl | H | acetyl | H | acetyl |
| 8446 | acetyl | acetyl | H | acetyl | H | H | acetyl | acetyl | H | acetyl | acetyl | H |
| 8447 | acetyl | acetyl | H | acetyl | H | H | acetyl | acetyl | H | acetyl | acetyl | acetyl |
| 8448 | acetyl | acetyl | H | acetyl | H | H | acetyl | acetyl | acetyl | H | H | H |
| 8449 | acetyl | acetyl | H | acetyl | H | H | acetyl | acetyl | acetyl | H | H | acetyl |
| 8450 | acetyl | acetyl | H | acetyl | H | H | acetyl | acetyl | acetyl | H | acetyl | H |
| 8451 | acetyl | acetyl | H | acetyl | H | H | acetyl | acetyl | acetyl | H | acetyl | acetyl |
| 8452 | acetyl | acetyl | H | acetyl | H | H | acetyl | acetyl | acetyl | acetyl | H | H |
| 8453 | acetyl | acetyl | H | acetyl | H | H | acetyl | acetyl | acetyl | acetyl | H | acetyl |
| 8454 | acetyl | acetyl | H | acetyl | H | H | acetyl | acetyl | acetyl | acetyl | acetyl | H |
| 8455 | acetyl | acetyl | H | acetyl | H | H | acetyl | acetyl | acetyl | acetyl | acetyl | acetyl |
| 8456 | acetyl | acetyl | H | acetyl | H | acetyl | H | H | H | H | H | H |
| 8457 | acetyl | acetyl | H | acetyl | H | acetyl | H | H | H | H | H | acetyl |
| 8458 | acetyl | acetyl | H | acetyl | H | acetyl | H | H | H | H | acetyl | H |
| 8459 | acetyl | acetyl | H | acetyl | H | acetyl | H | H | H | H | acetyl | acetyl |
| 8460 | acetyl | acetyl | H | acetyl | H | acetyl | H | H | H | acetyl | H | H |
| 8461 | acetyl | acetyl | H | acetyl | H | acetyl | H | H | H | acetyl | H | acetyl |
| 8462 | acetyl | acetyl | H | acetyl | H | acetyl | H | H | H | acetyl | acetyl | H |
| 8463 | acetyl | acetyl | H | acetyl | H | acetyl | H | H | H | acetyl | acetyl | acetyl |
| 8464 | acetyl | acetyl | H | acetyl | H | acetyl | H | H | acetyl | H | H | H |
| 8465 | acetyl | acetyl | H | acetyl | H | acetyl | H | H | acetyl | H | H | acetyl |
| 8466 | acetyl | acetyl | H | acetyl | H | acetyl | H | H | acetyl | H | acetyl | H |
| 8467 | acetyl | acetyl | H | acetyl | H | acetyl | H | H | acetyl | H | acetyl | acetyl |
| 8468 | acetyl | acetyl | H | acetyl | H | acetyl | H | H | acetyl | acetyl | H | H |
| 8469 | acetyl | acetyl | H | acetyl | H | acetyl | H | H | acetyl | acetyl | H | acetyl |
| 8470 | acetyl | acetyl | H | acetyl | H | acetyl | H | H | acetyl | acetyl | acetyl | H |
| 8471 | acetyl | acetyl | H | acetyl | H | acetyl | H | H | acetyl | acetyl | acetyl | acetyl |
| 8472 | acetyl | acetyl | H | acetyl | H | acetyl | H | acetyl | H | H | H | H |
| 8473 | acetyl | acetyl | H | acetyl | H | acetyl | H | acetyl | H | H | H | acetyl |
| 8474 | acetyl | acetyl | H | acetyl | H | acetyl | H | acetyl | H | H | acetyl | H |
| 8475 | acetyl | acetyl | H | acetyl | H | acetyl | H | acetyl | H | H | acetyl | acetyl |
| 8476 | acetyl | acetyl | H | acetyl | H | acetyl | H | acetyl | H | acetyl | H | H |
| 8477 | acetyl | acetyl | H | acetyl | H | acetyl | H | acetyl | H | acetyl | H | acetyl |
| 8478 | acetyl | acetyl | H | acetyl | H | acetyl | H | acetyl | H | acetyl | acetyl | H |
| 8479 | acetyl | acetyl | H | acetyl | H | acetyl | H | acetyl | H | acetyl | acetyl | acetyl |
| 8480 | acetyl | acetyl | H | acetyl | H | acetyl | H | acetyl | acetyl | H | H | H |
| 8481 | acetyl | acetyl | H | acetyl | H | acetyl | H | acetyl | acetyl | H | H | acetyl |
| 8482 | acetyl | acetyl | H | acetyl | H | acetyl | H | acetyl | acetyl | H | acetyl | H |
| 8483 | acetyl | acetyl | H | acetyl | H | acetyl | H | acetyl | acetyl | H | acetyl | acetyl |
| 8484 | acetyl | acetyl | H | acetyl | H | acetyl | H | acetyl | acetyl | acetyl | H | H |
| 8485 | acetyl | acetyl | H | acetyl | H | acetyl | H | acetyl | acetyl | acetyl | H | acetyl |
| 8486 | acetyl | acetyl | H | acetyl | H | acetyl | H | acetyl | acetyl | acetyl | acetyl | H |
| 8487 | acetyl | acetyl | H | acetyl | H | acetyl | H | acetyl | acetyl | acetyl | acetyl | acetyl |
| 8488 | acetyl | acetyl | H | acetyl | H | acetyl | acetyl | H | H | H | H | H |
| 8489 | acetyl | acetyl | H | acetyl | H | acetyl | acetyl | H | H | H | H | acetyl |
| 8490 | acetyl | acetyl | H | acetyl | H | acetyl | acetyl | H | H | H | acetyl | H |
| 8491 | acetyl | acetyl | H | acetyl | H | acetyl | acetyl | H | H | H | acetyl | acetyl |
| 8492 | acetyl | acetyl | H | acetyl | H | acetyl | acetyl | H | H | acetyl | H | H |
| 8493 | acetyl | acetyl | H | acetyl | H | acetyl | acetyl | H | H | acetyl | H | acetyl |
| 8494 | acetyl | acetyl | H | acetyl | H | acetyl | acetyl | H | H | acetyl | acetyl | H |
| 8495 | acetyl | acetyl | H | acetyl | H | acetyl | acetyl | H | H | acetyl | acetyl | acetyl |
| 8496 | acetyl | acetyl | H | acetyl | H | acetyl | acetyl | H | acetyl | H | H | H |
| 8497 | acetyl | acetyl | H | acetyl | H | acetyl | acetyl | H | acetyl | H | H | acetyl |
| 8498 | acetyl | acetyl | H | acetyl | H | acetyl | acetyl | H | acetyl | H | acetyl | H |
| 8499 | acetyl | acetyl | H | acetyl | H | acetyl | acetyl | H | acetyl | H | acetyl | acetyl |
| 8500 | acetyl | acetyl | H | acetyl | H | acetyl | acetyl | H | acetyl | acetyl | H | H |
| 8501 | acetyl | acetyl | H | acetyl | H | acetyl | acetyl | H | acetyl | acetyl | H | acetyl |
| 8502 | acetyl | acetyl | H | acetyl | H | acetyl | acetyl | H | acetyl | acetyl | acetyl | H |
| 8503 | acetyl | acetyl | H | acetyl | H | acetyl | acetyl | H | acetyl | acetyl | acetyl | acetyl |
| 8504 | acetyl | acetyl | H | acetyl | H | acetyl | acetyl | acetyl | H | H | H | H |
| 8505 | acetyl | acetyl | H | acetyl | H | acetyl | acetyl | acetyl | H | H | H | acetyl |
| 8506 | acetyl | acetyl | H | acetyl | H | acetyl | acetyl | acetyl | H | H | acetyl | H |
| 8507 | acetyl | acetyl | H | acetyl | H | acetyl | acetyl | acetyl | H | H | acetyl | acetyl |
| 8508 | acetyl | acetyl | H | acetyl | H | acetyl | acetyl | acetyl | H | acetyl | H | H |
| 8509 | acetyl | acetyl | H | acetyl | H | acetyl | acetyl | acetyl | H | acetyl | H | acetyl |
| 8510 | acetyl | acetyl | H | acetyl | H | acetyl | acetyl | acetyl | H | acetyl | acetyl | H |
| 8511 | acetyl | acetyl | H | acetyl | H | acetyl | acetyl | acetyl | H | acetyl | acetyl | acetyl |
| 8512 | acetyl | acetyl | H | acetyl | H | acetyl | acetyl | acetyl | acetyl | H | H | H |
| 8513 | acetyl | acetyl | H | acetyl | H | acetyl | acetyl | acetyl | acetyl | H | H | acetyl |
| 8514 | acetyl | acetyl | H | acetyl | H | acetyl | acetyl | acetyl | acetyl | H | acetyl | H |
| 8515 | acetyl | acetyl | H | acetyl | H | acetyl | acetyl | acetyl | acetyl | H | acetyl | acetyl |
| 8516 | acetyl | acetyl | H | acetyl | H | acetyl | acetyl | acetyl | acetyl | acetyl | H | H |
| 8517 | acetyl | acetyl | H | acetyl | H | acetyl | acetyl | acetyl | acetyl | acetyl | H | acetyl |
| 8518 | acetyl | acetyl | H | acetyl | H | acetyl | acetyl | acetyl | acetyl | acetyl | acetyl | H |
| 8519 | acetyl | acetyl | H | acetyl | H | acetyl | acetyl | acetyl | acetyl | acetyl | acetyl | acetyl |
| 8520 | acetyl | acetyl | H | acetyl | acetyl | H | H | H | H | H | H | H |
| 8521 | acetyl | acetyl | H | acetyl | acetyl | H | H | H | H | H | H | acetyl |
| 8522 | acetyl | acetyl | H | acetyl | acetyl | H | H | H | H | H | acetyl | H |
| 8523 | acetyl | acetyl | H | acetyl | acetyl | H | H | H | H | H | acetyl | acetyl |
| 8524 | acetyl | acetyl | H | acetyl | acetyl | H | H | H | H | acetyl | H | H |
| 8525 | acetyl | acetyl | H | acetyl | acetyl | H | H | H | H | acetyl | H | acetyl |
| 8526 | acetyl | acetyl | H | acetyl | acetyl | H | H | H | H | acetyl | acetyl | H |
| 8527 | acetyl | acetyl | H | acetyl | acetyl | H | H | H | H | acetyl | acetyl | acetyl |
| 8528 | acetyl | acetyl | H | acetyl | acetyl | H | H | H | acetyl | H | H | H |
| 8529 | acetyl | acetyl | H | acetyl | acetyl | H | H | H | acetyl | H | H | acetyl |
| 8530 | acetyl | acetyl | H | acetyl | acetyl | H | H | H | acetyl | H | acetyl | H |
| 8531 | acetyl | acetyl | H | acetyl | acetyl | H | H | H | acetyl | H | acetyl | acetyl |
| 8532 | acetyl | acetyl | H | acetyl | acetyl | H | H | H | acetyl | acetyl | H | H |
| 8533 | acetyl | acetyl | H | acetyl | acetyl | H | H | H | acetyl | acetyl | H | acetyl |
| 8534 | acetyl | acetyl | H | acetyl | acetyl | H | H | H | acetyl | acetyl | acetyl | H |
| 8535 | acetyl | acetyl | H | acetyl | acetyl | H | H | H | acetyl | acetyl | acetyl | acetyl |
| 8536 | acetyl | acetyl | H | acetyl | acetyl | H | H | acetyl | H | H | H | H |
| 8537 | acetyl | acetyl | H | acetyl | acetyl | H | H | acetyl | H | H | H | acetyl |
| 8538 | acetyl | acetyl | H | acetyl | acetyl | H | H | acetyl | H | H | acetyl | H |
| 8539 | acetyl | acetyl | H | acetyl | acetyl | H | H | acetyl | H | H | acetyl | acetyl |
| 8540 | acetyl | acetyl | H | acetyl | acetyl | H | H | acetyl | H | acetyl | H | H |
| 8541 | acetyl | acetyl | H | acetyl | acetyl | H | H | acetyl | H | acetyl | H | acetyl |
| 8542 | acetyl | acetyl | H | acetyl | acetyl | H | H | acetyl | H | acetyl | acetyl | H |
| 8543 | acetyl | acetyl | H | acetyl | acetyl | H | H | acetyl | H | acetyl | acetyl | acetyl |
| 8544 | acetyl | acetyl | H | acetyl | acetyl | H | H | acetyl | acetyl | H | H | H |
| 8545 | acetyl | acetyl | H | acetyl | acetyl | H | H | acetyl | acetyl | H | H | acetyl |
| 8546 | acetyl | acetyl | H | acetyl | acetyl | H | H | acetyl | acetyl | H | acetyl | H |
| 8547 | acetyl | acetyl | H | acetyl | acetyl | H | H | acetyl | acetyl | H | acetyl | acetyl |
| 8548 | acetyl | acetyl | H | acetyl | acetyl | H | H | acetyl | acetyl | acetyl | H | H |
| 8549 | acetyl | acetyl | H | acetyl | acetyl | H | H | acetyl | acetyl | acetyl | H | acetyl |
| 8550 | acetyl | acetyl | H | acetyl | acetyl | H | H | acetyl | acetyl | acetyl | acetyl | H |
| 8551 | acetyl | acetyl | H | acetyl | acetyl | H | H | acetyl | acetyl | acetyl | acetyl | acetyl |
| 8552 | acetyl | acetyl | H | acetyl | acetyl | H | acetyl | H | H | H | H | H |
| 8553 | acetyl | acetyl | H | acetyl | acetyl | H | acetyl | H | H | H | H | acetyl |
| 8554 | acetyl | acetyl | H | acetyl | acetyl | H | acetyl | H | H | H | acetyl | H |
| 8555 | acetyl | acetyl | H | acetyl | acetyl | H | acetyl | H | H | H | acetyl | acetyl |
| 8556 | acetyl | acetyl | H | acetyl | acetyl | H | acetyl | H | H | acetyl | H | H |
| 8557 | acetyl | acetyl | H | acetyl | acetyl | H | acetyl | H | H | acetyl | H | acetyl |
| 8558 | acetyl | acetyl | H | acetyl | acetyl | H | acetyl | H | H | acetyl | acetyl | H |
| 8559 | acetyl | acetyl | H | acetyl | acetyl | H | acetyl | H | H | acetyl | acetyl | acetyl |
| 8560 | acetyl | acetyl | H | acetyl | acetyl | H | acetyl | H | acetyl | H | H | H |
| 8561 | acetyl | acetyl | H | acetyl | acetyl | H | acetyl | H | acetyl | H | H | acetyl |
| 8562 | acetyl | acetyl | H | acetyl | acetyl | H | acetyl | H | acetyl | H | acetyl | H |
| 8563 | acetyl | acetyl | H | acetyl | acetyl | H | acetyl | H | acetyl | H | acetyl | acetyl |
| 8564 | acetyl | acetyl | H | acetyl | acetyl | H | acetyl | H | acetyl | acetyl | H | H |
| 8565 | acetyl | acetyl | H | acetyl | acetyl | H | acetyl | H | acetyl | acetyl | H | acetyl |
| 8566 | acetyl | acetyl | H | acetyl | acetyl | H | acetyl | H | acetyl | acetyl | acetyl | H |
| 8567 | acetyl | acetyl | H | acetyl | acetyl | H | acetyl | H | acetyl | acetyl | acetyl | acetyl |
| 8568 | acetyl | acetyl | H | acetyl | acetyl | H | acetyl | acetyl | H | H | H | H |
| 8569 | acetyl | acetyl | H | acetyl | acetyl | H | acetyl | acetyl | H | H | H | acetyl |
| 8570 | acetyl | acetyl | H | acetyl | acetyl | H | acetyl | acetyl | H | H | acetyl | H |
| 8571 | acetyl | acetyl | H | acetyl | acetyl | H | acetyl | acetyl | H | H | acetyl | acetyl |
| 8572 | acetyl | acetyl | H | acetyl | acetyl | H | acetyl | acetyl | H | acetyl | H | H |
| 8573 | acetyl | acetyl | H | acetyl | acetyl | H | acetyl | acetyl | H | acetyl | H | acetyl |
| 8574 | acetyl | acetyl | H | acetyl | acetyl | H | acetyl | acetyl | H | acetyl | acetyl | H |
| 8575 | acetyl | acetyl | H | acetyl | acetyl | H | acetyl | acetyl | H | acetyl | acetyl | acetyl |
| 8576 | acetyl | acetyl | H | acetyl | acetyl | H | acetyl | acetyl | acetyl | H | H | H |
| 8577 | acetyl | acetyl | H | acetyl | acetyl | H | acetyl | acetyl | acetyl | H | H | acetyl |
| 8578 | acetyl | acetyl | H | acetyl | acetyl | H | acetyl | acetyl | acetyl | H | acetyl | H |
| 8579 | acetyl | acetyl | H | acetyl | acetyl | H | acetyl | acetyl | acetyl | H | acetyl | acetyl |
| 8580 | acetyl | acetyl | H | acetyl | acetyl | H | acetyl | acetyl | acetyl | acetyl | H | H |
| 8581 | acetyl | acetyl | H | acetyl | acetyl | H | acetyl | acetyl | acetyl | acetyl | H | acetyl |
| 8582 | acetyl | acetyl | H | acetyl | acetyl | H | acetyl | acetyl | acetyl | acetyl | acetyl | H |
| 8583 | acetyl | acetyl | H | acetyl | acetyl | H | acetyl | acetyl | acetyl | acetyl | acetyl | acetyl |
| 8584 | acetyl | acetyl | H | acetyl | acetyl | acetyl | H | H | H | H | H | H |
| 8585 | acetyl | acetyl | H | acetyl | acetyl | acetyl | H | H | H | H | H | acetyl |
| 8586 | acetyl | acetyl | H | acetyl | acetyl | acetyl | H | H | H | H | acetyl | H |
| 8587 | acetyl | acetyl | H | acetyl | acetyl | acetyl | H | H | H | H | acetyl | acetyl |
| 8588 | acetyl | acetyl | H | acetyl | acetyl | acetyl | H | H | H | acetyl | H | H |
| 8589 | acetyl | acetyl | H | acetyl | acetyl | acetyl | H | H | H | acetyl | H | acetyl |
| 8590 | acetyl | acetyl | H | acetyl | acetyl | acetyl | H | H | H | acetyl | acetyl | H |
| 8591 | acetyl | acetyl | H | acetyl | acetyl | acetyl | H | H | H | acetyl | acetyl | acetyl |
| 8592 | acetyl | acetyl | H | acetyl | acetyl | acetyl | H | H | acetyl | H | H | H |
| 8593 | acetyl | acetyl | H | acetyl | acetyl | acetyl | H | H | acetyl | H | H | acetyl |
| 8594 | acetyl | acetyl | H | acetyl | acetyl | acetyl | H | H | acetyl | H | acetyl | H |
| 8595 | acetyl | acetyl | H | acetyl | acetyl | acetyl | H | H | acetyl | H | acetyl | acetyl |
| 8596 | acetyl | acetyl | H | acetyl | acetyl | acetyl | H | H | acetyl | acetyl | H | H |
| 8597 | acetyl | acetyl | H | acetyl | acetyl | acetyl | H | H | acetyl | acetyl | H | acetyl |
| 8598 | acetyl | acetyl | H | acetyl | acetyl | acetyl | H | H | acetyl | acetyl | acetyl | H |
| 8599 | acetyl | acetyl | H | acetyl | acetyl | acetyl | H | H | acetyl | acetyl | acetyl | acetyl |
| 8600 | acetyl | acetyl | H | acetyl | acetyl | acetyl | H | acetyl | H | H | H | H |
| 8601 | acetyl | acetyl | H | acetyl | acetyl | acetyl | H | acetyl | H | H | H | acetyl |
| 8602 | acetyl | acetyl | H | acetyl | acetyl | acetyl | H | acetyl | H | H | acetyl | H |
| 8603 | acetyl | acetyl | H | acetyl | acetyl | acetyl | H | acetyl | H | H | acetyl | acetyl |
| 8604 | acetyl | acetyl | H | acetyl | acetyl | acetyl | H | acetyl | H | acetyl | H | H |
| 8605 | acetyl | acetyl | H | acetyl | acetyl | acetyl | H | acetyl | H | acetyl | H | acetyl |
| 8606 | acetyl | acetyl | H | acetyl | acetyl | acetyl | H | acetyl | H | acetyl | acetyl | H |
| 8607 | acetyl | acetyl | H | acetyl | acetyl | acetyl | H | acetyl | H | acetyl | acetyl | acetyl |
| 8608 | acetyl | acetyl | H | acetyl | acetyl | acetyl | H | acetyl | acetyl | H | H | H |
| 8609 | acetyl | acetyl | H | acetyl | acetyl | acetyl | H | acetyl | acetyl | H | H | acetyl |
| 8610 | acetyl | acetyl | H | acetyl | acetyl | acetyl | H | acetyl | acetyl | H | acetyl | H |
| 8611 | acetyl | acetyl | H | acetyl | acetyl | acetyl | H | acetyl | acetyl | H | acetyl | acetyl |
| 8612 | acetyl | acetyl | H | acetyl | acetyl | acetyl | H | acetyl | acetyl | acetyl | H | H |
| 8613 | acetyl | acetyl | H | acetyl | acetyl | acetyl | H | acetyl | acetyl | acetyl | H | acetyl |
| 8614 | acetyl | acetyl | H | acetyl | acetyl | acetyl | H | acetyl | acetyl | acetyl | acetyl | H |
| 8615 | acetyl | acetyl | H | acetyl | acetyl | acetyl | H | acetyl | acetyl | acetyl | acetyl | acetyl |
| 8616 | acetyl | acetyl | H | acetyl | acetyl | acetyl | acetyl | H | H | H | H | H |
| 8617 | acetyl | acetyl | H | acetyl | acetyl | acetyl | acetyl | H | H | H | H | acetyl |
| 8618 | acetyl | acetyl | H | acetyl | acetyl | acetyl | acetyl | H | H | H | acetyl | H |
| 8619 | acetyl | acetyl | H | acetyl | acetyl | acetyl | acetyl | H | H | H | acetyl | acetyl |
| 8620 | acetyl | acetyl | H | acetyl | acetyl | acetyl | acetyl | H | H | acetyl | H | H |
| 8621 | acetyl | acetyl | H | acetyl | acetyl | acetyl | acetyl | H | H | acetyl | H | acetyl |
| 8622 | acetyl | acetyl | H | acetyl | acetyl | acetyl | acetyl | H | H | acetyl | acetyl | H |
| 8623 | acetyl | acetyl | H | acetyl | acetyl | acetyl | acetyl | H | H | acetyl | acetyl | acetyl |
| 8624 | acetyl | acetyl | H | acetyl | acetyl | acetyl | acetyl | H | acetyl | H | H | H |
| 8625 | acetyl | acetyl | H | acetyl | acetyl | acetyl | acetyl | H | acetyl | H | H | acetyl |
| 8626 | acetyl | acetyl | H | acetyl | acetyl | acetyl | acetyl | H | acetyl | H | acetyl | H |
| 8627 | acetyl | acetyl | H | acetyl | acetyl | acetyl | acetyl | H | acetyl | H | acetyl | acetyl |
| 8628 | acetyl | acetyl | H | acetyl | acetyl | acetyl | acetyl | H | acetyl | acetyl | H | H |
| 8629 | acetyl | acetyl | H | acetyl | acetyl | acetyl | acetyl | H | acetyl | acetyl | H | acetyl |
| 8630 | acetyl | acetyl | H | acetyl | acetyl | acetyl | acetyl | H | acetyl | acetyl | acetyl | H |
| 8631 | acetyl | acetyl | H | acetyl | acetyl | acetyl | acetyl | H | acetyl | acetyl | acetyl | acetyl |
| 8632 | acetyl | acetyl | H | acetyl | acetyl | acetyl | acetyl | acetyl | H | H | H | H |
| 8633 | acetyl | acetyl | H | acetyl | acetyl | acetyl | acetyl | acetyl | H | H | H | acetyl |
| 8634 | acetyl | acetyl | H | acetyl | acetyl | acetyl | acetyl | acetyl | H | H | acetyl | H |
| 8635 | acetyl | acetyl | H | acetyl | acetyl | acetyl | acetyl | acetyl | H | H | acetyl | acetyl |
| 8636 | acetyl | acetyl | H | acetyl | acetyl | acetyl | acetyl | acetyl | H | acetyl | H | H |
| 8637 | acetyl | acetyl | H | acetyl | acetyl | acetyl | acetyl | acetyl | H | acetyl | H | acetyl |
| 8638 | acetyl | acetyl | H | acetyl | acetyl | acetyl | acetyl | acetyl | H | acetyl | acetyl | H |
| 8639 | acetyl | acetyl | H | acetyl | acetyl | acetyl | acetyl | acetyl | H | acetyl | acetyl | acetyl |
| 8640 | acetyl | acetyl | H | acetyl | acetyl | acetyl | acetyl | acetyl | acetyl | H | H | H |
| 8641 | acetyl | acetyl | H | acetyl | acetyl | acetyl | acetyl | acetyl | acetyl | H | H | acetyl |
| 8642 | acetyl | acetyl | H | acetyl | acetyl | acetyl | acetyl | acetyl | acetyl | H | acetyl | H |
| 8643 | acetyl | acetyl | H | acetyl | acetyl | acetyl | acetyl | acetyl | acetyl | H | acetyl | acetyl |
| 8644 | acetyl | acetyl | H | acetyl | acetyl | acetyl | acetyl | acetyl | acetyl | acetyl | H | H |
| 8645 | acetyl | acetyl | H | acetyl | acetyl | acetyl | acetyl | acetyl | acetyl | acetyl | H | acetyl |
| 8646 | acetyl | acetyl | H | acetyl | acetyl | acetyl | acetyl | acetyl | acetyl | acetyl | acetyl | H |
| 8647 | acetyl | acetyl | H | acetyl | acetyl | acetyl | acetyl | acetyl | acetyl | acetyl | acetyl | acetyl |
| 8648 | acetyl | acetyl | acetyl | H | H | H | H | H | H | H | H | H |
| 8649 | acetyl | acetyl | acetyl | H | H | H | H | H | H | H | H | acetyl |
| 8650 | acetyl | acetyl | acetyl | H | H | H | H | H | H | H | acetyl | H |
| 8651 | acetyl | acetyl | acetyl | H | H | H | H | H | H | H | acetyl | acetyl |
| 8652 | acetyl | acetyl | acetyl | H | H | H | H | H | H | acetyl | H | H |
| 8653 | acetyl | acetyl | acetyl | H | H | H | H | H | H | acetyl | H | acetyl |
| 8654 | acetyl | acetyl | acetyl | H | H | H | H | H | H | acetyl | acetyl | H |
| 8655 | acetyl | acetyl | acetyl | H | H | H | H | H | H | acetyl | acetyl | acetyl |
| 8656 | acetyl | acetyl | acetyl | H | H | H | H | H | acetyl | H | H | H |
| 8657 | acetyl | acetyl | acetyl | H | H | H | H | H | acetyl | H | H | acetyl |
| 8658 | acetyl | acetyl | acetyl | H | H | H | H | H | acetyl | H | acetyl | H |
| 8659 | acetyl | acetyl | acetyl | H | H | H | H | H | acetyl | H | acetyl | acetyl |
| 8660 | acetyl | acetyl | acetyl | H | H | H | H | H | acetyl | acetyl | H | H |
| 8661 | acetyl | acetyl | acetyl | H | H | H | H | H | acetyl | acetyl | H | acetyl |
| 8662 | acetyl | acetyl | acetyl | H | H | H | H | H | acetyl | acetyl | acetyl | H |
| 8663 | acetyl | acetyl | acetyl | H | H | H | H | H | acetyl | acetyl | acetyl | acetyl |
| 8664 | acetyl | acetyl | acetyl | H | H | H | H | acetyl | H | H | H | H |
| 8665 | acetyl | acetyl | acetyl | H | H | H | H | acetyl | H | H | H | acetyl |
| 8666 | acetyl | acetyl | acetyl | H | H | H | H | acetyl | H | H | acetyl | H |
| 8667 | acetyl | acetyl | acetyl | H | H | H | H | acetyl | H | H | acetyl | acetyl |
| 8668 | acetyl | acetyl | acetyl | H | H | H | H | acetyl | H | acetyl | H | H |
| 8669 | acetyl | acetyl | acetyl | H | H | H | H | acetyl | H | acetyl | H | acetyl |
| 8670 | acetyl | acetyl | acetyl | H | H | H | H | acetyl | H | acetyl | acetyl | H |
| 8671 | acetyl | acetyl | acetyl | H | H | H | H | acetyl | H | acetyl | acetyl | acetyl |
| 8672 | acetyl | acetyl | acetyl | H | H | H | H | acetyl | acetyl | H | H | H |
| 8673 | acetyl | acetyl | acetyl | H | H | H | H | acetyl | acetyl | H | H | acetyl |
| 8674 | acetyl | acetyl | acetyl | H | H | H | H | acetyl | acetyl | H | acetyl | H |
| 8675 | acetyl | acetyl | acetyl | H | H | H | H | acetyl | acetyl | H | acetyl | acetyl |
| 8676 | acetyl | acetyl | acetyl | H | H | H | H | acetyl | acetyl | acetyl | H | H |
| 8677 | acetyl | acetyl | acetyl | H | H | H | H | acetyl | acetyl | acetyl | H | acetyl |
| 8678 | acetyl | acetyl | acetyl | H | H | H | H | acetyl | acetyl | acetyl | acetyl | H |
| 8679 | acetyl | acetyl | acetyl | H | H | H | H | acetyl | acetyl | acetyl | acetyl | acetyl |
| 8680 | acetyl | acetyl | acetyl | H | H | H | acetyl | H | H | H | H | H |
| 8681 | acetyl | acetyl | acetyl | H | H | H | acetyl | H | H | H | H | acetyl |
| 8682 | acetyl | acetyl | acetyl | H | H | H | acetyl | H | H | H | acetyl | H |
| 8683 | acetyl | acetyl | acetyl | H | H | H | acetyl | H | H | H | acetyl | acetyl |
| 8684 | acetyl | acetyl | acetyl | H | H | H | acetyl | H | H | acetyl | H | H |
| 8685 | acetyl | acetyl | acetyl | H | H | H | acetyl | H | H | acetyl | H | acetyl |
| 8686 | acetyl | acetyl | acetyl | H | H | H | acetyl | H | H | acetyl | acetyl | H |
| 8687 | acetyl | acetyl | acetyl | H | H | H | acetyl | H | H | acetyl | acetyl | acetyl |
| 8688 | acetyl | acetyl | acetyl | H | H | H | acetyl | H | acetyl | H | H | H |
| 8689 | acetyl | acetyl | acetyl | H | H | H | acetyl | H | acetyl | H | H | acetyl |
| 8690 | acetyl | acetyl | acetyl | H | H | H | acetyl | H | acetyl | H | acetyl | H |
| 8691 | acetyl | acetyl | acetyl | H | H | H | acetyl | H | acetyl | H | acetyl | acetyl |
| 8692 | acetyl | acetyl | acetyl | H | H | H | acetyl | H | acetyl | acetyl | H | H |
| 8693 | acetyl | acetyl | acetyl | H | H | H | acetyl | H | acetyl | acetyl | H | acetyl |
| 8694 | acetyl | acetyl | acetyl | H | H | H | acetyl | H | acetyl | acetyl | acetyl | H |
| 8695 | acetyl | acetyl | acetyl | H | H | H | acetyl | H | acetyl | acetyl | acetyl | acetyl |
| 8696 | acetyl | acetyl | acetyl | H | H | H | acetyl | acetyl | H | H | H | H |
| 8697 | acetyl | acetyl | acetyl | H | H | H | acetyl | acetyl | H | H | H | acetyl |
| 8698 | acetyl | acetyl | acetyl | H | H | H | acetyl | acetyl | H | H | acetyl | H |
| 8699 | acetyl | acetyl | acetyl | H | H | H | acetyl | acetyl | H | H | acetyl | acetyl |
| 8700 | acetyl | acetyl | acetyl | H | H | H | acetyl | acetyl | H | acetyl | H | H |
| 8701 | acetyl | acetyl | acetyl | H | H | H | acetyl | acetyl | H | acetyl | H | acetyl |
| 8702 | acetyl | acetyl | acetyl | H | H | H | acetyl | acetyl | H | acetyl | acetyl | H |
| 8703 | acetyl | acetyl | acetyl | H | H | H | acetyl | acetyl | H | acetyl | acetyl | acetyl |
| 8704 | acetyl | acetyl | acetyl | H | H | H | acetyl | acetyl | acetyl | H | H | H |
| 8705 | acetyl | acetyl | acetyl | H | H | H | acetyl | acetyl | acetyl | H | H | acetyl |
| 8706 | acetyl | acetyl | acetyl | H | H | H | acetyl | acetyl | acetyl | H | acetyl | H |
| 8707 | acetyl | acetyl | acetyl | H | H | H | acetyl | acetyl | acetyl | H | acetyl | acetyl |
| 8708 | acetyl | acetyl | acetyl | H | H | H | acetyl | acetyl | acetyl | acetyl | H | H |
| 8709 | acetyl | acetyl | acetyl | H | H | H | acetyl | acetyl | acetyl | acetyl | H | acetyl |
| 8710 | acetyl | acetyl | acetyl | H | H | H | acetyl | acetyl | acetyl | acetyl | acetyl | H |
| 8711 | acetyl | acetyl | acetyl | H | H | H | acetyl | acetyl | acetyl | acetyl | acetyl | acetyl |
| 8712 | acetyl | acetyl | acetyl | H | H | acetyl | H | H | H | H | H | H |
| 8713 | acetyl | acetyl | acetyl | H | H | acetyl | H | H | H | H | H | acetyl |
| 8714 | acetyl | acetyl | acetyl | H | H | acetyl | H | H | H | H | acetyl | H |
| 8715 | acetyl | acetyl | acetyl | H | H | acetyl | H | H | H | H | acetyl | acetyl |
| 8716 | acetyl | acetyl | acetyl | H | H | acetyl | H | H | H | acetyl | H | H |
| 8717 | acetyl | acetyl | acetyl | H | H | acetyl | H | H | H | acetyl | H | acetyl |
| 8718 | acetyl | acetyl | acetyl | H | H | acetyl | H | H | H | acetyl | acetyl | H |
| 8719 | acetyl | acetyl | acetyl | H | H | acetyl | H | H | H | acetyl | acetyl | acetyl |
| 8720 | acetyl | acetyl | acetyl | H | H | acetyl | H | H | acetyl | H | H | H |
| 8721 | acetyl | acetyl | acetyl | H | H | acetyl | H | H | acetyl | H | H | acetyl |
| 8722 | acetyl | acetyl | acetyl | H | H | acetyl | H | H | acetyl | H | acetyl | H |
| 8723 | acetyl | acetyl | acetyl | H | H | acetyl | H | H | acetyl | H | acetyl | acetyl |
| 8724 | acetyl | acetyl | acetyl | H | H | acetyl | H | H | acetyl | acetyl | H | H |
| 8725 | acetyl | acetyl | acetyl | H | H | acetyl | H | H | acetyl | acetyl | H | acetyl |
| 8726 | acetyl | acetyl | acetyl | H | H | acetyl | H | H | acetyl | acetyl | acetyl | H |
| 8727 | acetyl | acetyl | acetyl | H | H | acetyl | H | H | acetyl | acetyl | acetyl | acetyl |
| 8728 | acetyl | acetyl | acetyl | H | H | acetyl | H | acetyl | H | H | H | H |
| 8729 | acetyl | acetyl | acetyl | H | H | acetyl | H | acetyl | H | H | H | acetyl |
| 8730 | acetyl | acetyl | acetyl | H | H | acetyl | H | acetyl | H | H | acetyl | H |
| 8731 | acetyl | acetyl | acetyl | H | H | acetyl | H | acetyl | H | H | acetyl | acetyl |
| 8732 | acetyl | acetyl | acetyl | H | H | acetyl | H | acetyl | H | acetyl | H | H |
| 8733 | acetyl | acetyl | acetyl | H | H | acetyl | H | acetyl | H | acetyl | H | acetyl |
| 8734 | acetyl | acetyl | acetyl | H | H | acetyl | H | acetyl | H | acetyl | acetyl | H |
| 8735 | acetyl | acetyl | acetyl | H | H | acetyl | H | acetyl | H | acetyl | acetyl | acetyl |
| 8736 | acetyl | acetyl | acetyl | H | H | acetyl | H | acetyl | acetyl | H | H | H |
| 8737 | acetyl | acetyl | acetyl | H | H | acetyl | H | acetyl | acetyl | H | H | acetyl |
| 8738 | acetyl | acetyl | acetyl | H | H | acetyl | H | acetyl | acetyl | H | acetyl | H |
| 8739 | acetyl | acetyl | acetyl | H | H | acetyl | H | acetyl | acetyl | H | acetyl | acetyl |
| 8740 | acetyl | acetyl | acetyl | H | H | acetyl | H | acetyl | acetyl | acetyl | H | H |
| 8741 | acetyl | acetyl | acetyl | H | H | acetyl | H | acetyl | acetyl | acetyl | H | acetyl |
| 8742 | acetyl | acetyl | acetyl | H | H | acetyl | H | acetyl | acetyl | acetyl | acetyl | H |
| 8743 | acetyl | acetyl | acetyl | H | H | acetyl | H | acetyl | acetyl | acetyl | acetyl | acetyl |
| 8744 | acetyl | acetyl | acetyl | H | H | acetyl | acetyl | H | H | H | H | H |
| 8745 | acetyl | acetyl | acetyl | H | H | acetyl | acetyl | H | H | H | H | acetyl |
| 8746 | acetyl | acetyl | acetyl | H | H | acetyl | acetyl | H | H | H | acetyl | H |
| 8747 | acetyl | acetyl | acetyl | H | H | acetyl | acetyl | H | H | H | acetyl | acetyl |
| 8748 | acetyl | acetyl | acetyl | H | H | acetyl | acetyl | H | H | acetyl | H | H |
| 8749 | acetyl | acetyl | acetyl | H | H | acetyl | acetyl | H | H | acetyl | H | acetyl |
| 8750 | acetyl | acetyl | acetyl | H | H | acetyl | acetyl | H | H | acetyl | acetyl | H |
| 8751 | acetyl | acetyl | acetyl | H | H | acetyl | acetyl | H | H | acetyl | acetyl | acetyl |
| 8752 | acetyl | acetyl | acetyl | H | H | acetyl | acetyl | H | acetyl | H | H | H |
| 8753 | acetyl | acetyl | acetyl | H | H | acetyl | acetyl | H | acetyl | H | H | acetyl |
| 8754 | acetyl | acetyl | acetyl | H | H | acetyl | acetyl | H | acetyl | H | acetyl | H |
| 8755 | acetyl | acetyl | acetyl | H | H | acetyl | acetyl | H | acetyl | H | acetyl | acetyl |
| 8756 | acetyl | acetyl | acetyl | H | H | acetyl | acetyl | H | acetyl | acetyl | H | H |
| 8757 | acetyl | acetyl | acetyl | H | H | acetyl | acetyl | H | acetyl | acetyl | H | acetyl |
| 8758 | acetyl | acetyl | acetyl | H | H | acetyl | acetyl | H | acetyl | acetyl | acetyl | H |
| 8759 | acetyl | acetyl | acetyl | H | H | acetyl | acetyl | H | acetyl | acetyl | acetyl | acetyl |
| 8760 | acetyl | acetyl | acetyl | H | H | acetyl | acetyl | acetyl | H | H | H | H |
| 8761 | acetyl | acetyl | acetyl | H | H | acetyl | acetyl | acetyl | H | H | H | acetyl |
| 8762 | acetyl | acetyl | acetyl | H | H | acetyl | acetyl | acetyl | H | H | acetyl | H |
| 8763 | acetyl | acetyl | acetyl | H | H | acetyl | acetyl | acetyl | H | H | acetyl | acetyl |
| 8764 | acetyl | acetyl | acetyl | H | H | acetyl | acetyl | acetyl | H | acetyl | H | H |
| 8765 | acetyl | acetyl | acetyl | H | H | acetyl | acetyl | acetyl | H | acetyl | H | acetyl |
| 8766 | acetyl | acetyl | acetyl | H | H | acetyl | acetyl | acetyl | H | acetyl | acetyl | H |
| 8767 | acetyl | acetyl | acetyl | H | H | acetyl | acetyl | acetyl | H | acetyl | acetyl | acetyl |
| 8768 | acetyl | acetyl | acetyl | H | H | acetyl | acetyl | acetyl | acetyl | H | H | H |
| 8769 | acetyl | acetyl | acetyl | H | H | acetyl | acetyl | acetyl | acetyl | H | H | acetyl |
| 8770 | acetyl | acetyl | acetyl | H | H | acetyl | acetyl | acetyl | acetyl | H | acetyl | H |
| 8771 | acetyl | acetyl | acetyl | H | H | acetyl | acetyl | acetyl | acetyl | H | acetyl | acetyl |
| 8772 | acetyl | acetyl | acetyl | H | H | acetyl | acetyl | acetyl | acetyl | acetyl | H | H |
| 8773 | acetyl | acetyl | acetyl | H | H | acetyl | acetyl | acetyl | acetyl | acetyl | H | acetyl |
| 8774 | acetyl | acetyl | acetyl | H | H | acetyl | acetyl | acetyl | acetyl | acetyl | acetyl | H |
| 8775 | acetyl | acetyl | acetyl | H | H | acetyl | acetyl | acetyl | acetyl | acetyl | acetyl | acetyl |
| 8776 | acetyl | acetyl | acetyl | H | acetyl | H | H | H | H | H | H | H |
| 8777 | acetyl | acetyl | acetyl | H | acetyl | H | H | H | H | H | H | acetyl |
| 8778 | acetyl | acetyl | acetyl | H | acetyl | H | H | H | H | H | acetyl | H |
| 8779 | acetyl | acetyl | acetyl | H | acetyl | H | H | H | H | H | acetyl | acetyl |
| 8780 | acetyl | acetyl | acetyl | H | acetyl | H | H | H | H | acetyl | H | H |
| 8781 | acetyl | acetyl | acetyl | H | acetyl | H | H | H | H | acetyl | H | acetyl |
| 8782 | acetyl | acetyl | acetyl | H | acetyl | H | H | H | H | acetyl | acetyl | H |
| 8783 | acetyl | acetyl | acetyl | H | acetyl | H | H | H | H | acetyl | acetyl | acetyl |
| 8784 | acetyl | acetyl | acetyl | H | acetyl | H | H | H | acetyl | H | H | H |
| 8785 | acetyl | acetyl | acetyl | H | acetyl | H | H | H | acetyl | H | H | acetyl |
| 8786 | acetyl | acetyl | acetyl | H | acetyl | H | H | H | acetyl | H | acetyl | H |
| 8787 | acetyl | acetyl | acetyl | H | acetyl | H | H | H | acetyl | H | acetyl | acetyl |
| 8788 | acetyl | acetyl | acetyl | H | acetyl | H | H | H | acetyl | acetyl | H | H |
| 8789 | acetyl | acetyl | acetyl | H | acetyl | H | H | H | acetyl | acetyl | H | acetyl |
| 8790 | acetyl | acetyl | acetyl | H | acetyl | H | H | H | acetyl | acetyl | acetyl | H |
| 8791 | acetyl | acetyl | acetyl | H | acetyl | H | H | H | acetyl | acetyl | acetyl | acetyl |
| 8792 | acetyl | acetyl | acetyl | H | acetyl | H | H | acetyl | H | H | H | H |
| 8793 | acetyl | acetyl | acetyl | H | acetyl | H | H | acetyl | H | H | H | acetyl |
| 8794 | acetyl | acetyl | acetyl | H | acetyl | H | H | acetyl | H | H | acetyl | H |
| 8795 | acetyl | acetyl | acetyl | H | acetyl | H | H | acetyl | H | H | acetyl | acetyl |
| 8796 | acetyl | acetyl | acetyl | H | acetyl | H | H | acetyl | H | acetyl | H | H |
| 8797 | acetyl | acetyl | acetyl | H | acetyl | H | H | acetyl | H | acetyl | H | acetyl |
| 8798 | acetyl | acetyl | acetyl | H | acetyl | H | H | acetyl | H | acetyl | acetyl | H |
| 8799 | acetyl | acetyl | acetyl | H | acetyl | H | H | acetyl | H | acetyl | acetyl | acetyl |
| 8800 | acetyl | acetyl | acetyl | H | acetyl | H | H | acetyl | acetyl | H | H | H |
| 8801 | acetyl | acetyl | acetyl | H | acetyl | H | H | acetyl | acetyl | H | H | acetyl |
| 8802 | acetyl | acetyl | acetyl | H | acetyl | H | H | acetyl | acetyl | H | acetyl | H |
| 8803 | acetyl | acetyl | acetyl | H | acetyl | H | H | acetyl | acetyl | H | acetyl | acetyl |
| 8804 | acetyl | acetyl | acetyl | H | acetyl | H | H | acetyl | acetyl | acetyl | H | H |
| 8805 | acetyl | acetyl | acetyl | H | acetyl | H | H | acetyl | acetyl | acetyl | H | acetyl |
| 8806 | acetyl | acetyl | acetyl | H | acetyl | H | H | acetyl | acetyl | acetyl | acetyl | H |
| 8807 | acetyl | acetyl | acetyl | H | acetyl | H | H | acetyl | acetyl | acetyl | acetyl | acetyl |
| 8808 | acetyl | acetyl | acetyl | H | acetyl | H | acetyl | H | H | H | H | H |
| 8809 | acetyl | acetyl | acetyl | H | acetyl | H | acetyl | H | H | H | H | acetyl |
| 8810 | acetyl | acetyl | acetyl | H | acetyl | H | acetyl | H | H | H | acetyl | H |
| 8811 | acetyl | acetyl | acetyl | H | acetyl | H | acetyl | H | H | H | acetyl | acetyl |
| 8812 | acetyl | acetyl | acetyl | H | acetyl | H | acetyl | H | H | acetyl | H | H |
| 8813 | acetyl | acetyl | acetyl | H | acetyl | H | acetyl | H | H | acetyl | H | acetyl |
| 8814 | acetyl | acetyl | acetyl | H | acetyl | H | acetyl | H | H | acetyl | acetyl | H |
| 8815 | acetyl | acetyl | acetyl | H | acetyl | H | acetyl | H | H | acetyl | acetyl | acetyl |
| 8816 | acetyl | acetyl | acetyl | H | acetyl | H | acetyl | H | acetyl | H | H | H |
| 8817 | acetyl | acetyl | acetyl | H | acetyl | H | acetyl | H | acetyl | H | H | acetyl |
| 8818 | acetyl | acetyl | acetyl | H | acetyl | H | acetyl | H | acetyl | H | acetyl | H |
| 8819 | acetyl | acetyl | acetyl | H | acetyl | H | acetyl | H | acetyl | H | acetyl | acetyl |
| 8820 | acetyl | acetyl | acetyl | H | acetyl | H | acetyl | H | acetyl | acetyl | H | H |
| 8821 | acetyl | acetyl | acetyl | H | acetyl | H | acetyl | H | acetyl | acetyl | H | acetyl |
| 8822 | acetyl | acetyl | acetyl | H | acetyl | H | acetyl | H | acetyl | acetyl | acetyl | H |
| 8823 | acetyl | acetyl | acetyl | H | acetyl | H | acetyl | H | acetyl | acetyl | acetyl | acetyl |
| 8824 | acetyl | acetyl | acetyl | H | acetyl | H | acetyl | acetyl | H | H | H | H |
| 8825 | acetyl | acetyl | acetyl | H | acetyl | H | acetyl | acetyl | H | H | H | acetyl |
| 8826 | acetyl | acetyl | acetyl | H | acetyl | H | acetyl | acetyl | H | H | acetyl | H |
| 8827 | acetyl | acetyl | acetyl | H | acetyl | H | acetyl | acetyl | H | H | acetyl | acetyl |
| 8828 | acetyl | acetyl | acetyl | H | acetyl | H | acetyl | acetyl | H | acetyl | H | H |
| 8829 | acetyl | acetyl | acetyl | H | acetyl | H | acetyl | acetyl | H | acetyl | H | acetyl |
| 8830 | acetyl | acetyl | acetyl | H | acetyl | H | acetyl | acetyl | H | acetyl | acetyl | H |
| 8831 | acetyl | acetyl | acetyl | H | acetyl | H | acetyl | acetyl | H | acetyl | acetyl | acetyl |
| 8832 | acetyl | acetyl | acetyl | H | acetyl | H | acetyl | acetyl | acetyl | H | H | H |
| 8833 | acetyl | acetyl | acetyl | H | acetyl | H | acetyl | acetyl | acetyl | H | H | acetyl |
| 8834 | acetyl | acetyl | acetyl | H | acetyl | H | acetyl | acetyl | acetyl | H | acetyl | H |
| 8835 | acetyl | acetyl | acetyl | H | acetyl | H | acetyl | acetyl | acetyl | H | acetyl | acetyl |
| 8836 | acetyl | acetyl | acetyl | H | acetyl | H | acetyl | acetyl | acetyl | acetyl | H | H |
| 8837 | acetyl | acetyl | acetyl | H | acetyl | H | acetyl | acetyl | acetyl | acetyl | H | acetyl |
| 8838 | acetyl | acetyl | acetyl | H | acetyl | H | acetyl | acetyl | acetyl | acetyl | acetyl | H |
| 8839 | acetyl | acetyl | acetyl | H | acetyl | H | acetyl | acetyl | acetyl | acetyl | acetyl | acetyl |
| 8840 | acetyl | acetyl | acetyl | H | acetyl | acetyl | H | H | H | H | H | H |
| 8841 | acetyl | acetyl | acetyl | H | acetyl | acetyl | H | H | H | H | H | acetyl |
| 8842 | acetyl | acetyl | acetyl | H | acetyl | acetyl | H | H | H | H | acetyl | H |
| 8843 | acetyl | acetyl | acetyl | H | acetyl | acetyl | H | H | H | H | acetyl | acetyl |
| 8844 | acetyl | acetyl | acetyl | H | acetyl | acetyl | H | H | H | acetyl | H | H |
| 8845 | acetyl | acetyl | acetyl | H | acetyl | acetyl | H | H | H | acetyl | H | acetyl |
| 8846 | acetyl | acetyl | acetyl | H | acetyl | acetyl | H | H | H | acetyl | acetyl | H |
| 8847 | acetyl | acetyl | acetyl | H | acetyl | acetyl | H | H | H | acetyl | acetyl | acetyl |
| 8848 | acetyl | acetyl | acetyl | H | acetyl | acetyl | H | H | acetyl | H | H | H |
| 8849 | acetyl | acetyl | acetyl | H | acetyl | acetyl | H | H | acetyl | H | H | acetyl |
| 8850 | acetyl | acetyl | acetyl | H | acetyl | acetyl | H | H | acetyl | H | acetyl | H |
| 8851 | acetyl | acetyl | acetyl | H | acetyl | acetyl | H | H | acetyl | H | acetyl | acetyl |
| 8852 | acetyl | acetyl | acetyl | H | acetyl | acetyl | H | H | acetyl | acetyl | H | H |
| 8853 | acetyl | acetyl | acetyl | H | acetyl | acetyl | H | H | acetyl | acetyl | H | acetyl |
| 8854 | acetyl | acetyl | acetyl | H | acetyl | acetyl | H | H | acetyl | acetyl | acetyl | H |
| 8855 | acetyl | acetyl | acetyl | H | acetyl | acetyl | H | H | acetyl | acetyl | acetyl | acetyl |
| 8856 | acetyl | acetyl | acetyl | H | acetyl | acetyl | H | acetyl | H | H | H | H |
| 8857 | acetyl | acetyl | acetyl | H | acetyl | acetyl | H | acetyl | H | H | H | acetyl |
| 8858 | acetyl | acetyl | acetyl | H | acetyl | acetyl | H | acetyl | H | H | acetyl | H |
| 8859 | acetyl | acetyl | acetyl | H | acetyl | acetyl | H | acetyl | H | H | acetyl | acetyl |
| 8860 | acetyl | acetyl | acetyl | H | acetyl | acetyl | H | acetyl | H | acetyl | H | H |
| 8861 | acetyl | acetyl | acetyl | H | acetyl | acetyl | H | acetyl | H | acetyl | H | acetyl |
| 8862 | acetyl | acetyl | acetyl | H | acetyl | acetyl | H | acetyl | H | acetyl | acetyl | H |
| 8863 | acetyl | acetyl | acetyl | H | acetyl | acetyl | H | acetyl | H | acetyl | acetyl | acetyl |
| 8864 | acetyl | acetyl | acetyl | H | acetyl | acetyl | H | acetyl | acetyl | H | H | H |
| 8865 | acetyl | acetyl | acetyl | H | acetyl | acetyl | H | acetyl | acetyl | H | H | acetyl |
| 8866 | acetyl | acetyl | acetyl | H | acetyl | acetyl | H | acetyl | acetyl | H | acetyl | H |
| 8867 | acetyl | acetyl | acetyl | H | acetyl | acetyl | H | acetyl | acetyl | H | acetyl | acetyl |
| 8868 | acetyl | acetyl | acetyl | H | acetyl | acetyl | H | acetyl | acetyl | acetyl | H | H |
| 8869 | acetyl | acetyl | acetyl | H | acetyl | acetyl | H | acetyl | acetyl | acetyl | H | acetyl |
| 8870 | acetyl | acetyl | acetyl | H | acetyl | acetyl | H | acetyl | acetyl | acetyl | acetyl | H |
| 8871 | acetyl | acetyl | acetyl | H | acetyl | acetyl | H | acetyl | acetyl | acetyl | acetyl | acetyl |
| 8872 | acetyl | acetyl | acetyl | H | acetyl | acetyl | acetyl | H | H | H | H | H |
| 8873 | acetyl | acetyl | acetyl | H | acetyl | acetyl | acetyl | H | H | H | H | acetyl |
| 8874 | acetyl | acetyl | acetyl | H | acetyl | acetyl | acetyl | H | H | H | acetyl | H |
| 8875 | acetyl | acetyl | acetyl | H | acetyl | acetyl | acetyl | H | H | H | acetyl | acetyl |
| 8876 | acetyl | acetyl | acetyl | H | acetyl | acetyl | acetyl | H | H | acetyl | H | H |
| 8877 | acetyl | acetyl | acetyl | H | acetyl | acetyl | acetyl | H | H | acetyl | H | acetyl |
| 8878 | acetyl | acetyl | acetyl | H | acetyl | acetyl | acetyl | H | H | acetyl | acetyl | H |
| 8879 | acetyl | acetyl | acetyl | H | acetyl | acetyl | acetyl | H | H | acetyl | acetyl | acetyl |
| 8880 | acetyl | acetyl | acetyl | H | acetyl | acetyl | acetyl | H | acetyl | H | H | H |
| 8881 | acetyl | acetyl | acetyl | H | acetyl | acetyl | acetyl | H | acetyl | H | H | acetyl |
| 8882 | acetyl | acetyl | acetyl | H | acetyl | acetyl | acetyl | H | acetyl | H | acetyl | H |
| 8883 | acetyl | acetyl | acetyl | H | acetyl | acetyl | acetyl | H | acetyl | H | acetyl | acetyl |
| 8884 | acetyl | acetyl | acetyl | H | acetyl | acetyl | acetyl | H | acetyl | acetyl | H | H |
| 8885 | acetyl | acetyl | acetyl | H | acetyl | acetyl | acetyl | H | acetyl | acetyl | H | acetyl |
| 8886 | acetyl | acetyl | acetyl | H | acetyl | acetyl | acetyl | H | acetyl | acetyl | acetyl | H |
| 8887 | acetyl | acetyl | acetyl | H | acetyl | acetyl | acetyl | H | acetyl | acetyl | acetyl | acetyl |
| 8888 | acetyl | acetyl | acetyl | H | acetyl | acetyl | acetyl | acetyl | H | H | H | H |
| 8889 | acetyl | acetyl | acetyl | H | acetyl | acetyl | acetyl | acetyl | H | H | H | acetyl |
| 8890 | acetyl | acetyl | acetyl | H | acetyl | acetyl | acetyl | acetyl | H | H | acetyl | H |
| 8891 | acetyl | acetyl | acetyl | H | acetyl | acetyl | acetyl | acetyl | H | H | acetyl | acetyl |
| 8892 | acetyl | acetyl | acetyl | H | acetyl | acetyl | acetyl | acetyl | H | acetyl | H | H |
| 8893 | acetyl | acetyl | acetyl | H | acetyl | acetyl | acetyl | acetyl | H | acetyl | H | acetyl |
| 8894 | acetyl | acetyl | acetyl | H | acetyl | acetyl | acetyl | acetyl | H | acetyl | acetyl | H |
| 8895 | acetyl | acetyl | acetyl | H | acetyl | acetyl | acetyl | acetyl | H | acetyl | acetyl | acetyl |
| 8896 | acetyl | acetyl | acetyl | H | acetyl | acetyl | acetyl | acetyl | acetyl | H | H | H |
| 8897 | acetyl | acetyl | acetyl | H | acetyl | acetyl | acetyl | acetyl | acetyl | H | H | acetyl |
| 8898 | acetyl | acetyl | acetyl | H | acetyl | acetyl | acetyl | acetyl | acetyl | H | acetyl | H |
| 8899 | acetyl | acetyl | acetyl | H | acetyl | acetyl | acetyl | acetyl | acetyl | H | acetyl | acetyl |
| 8900 | acetyl | acetyl | acetyl | H | acetyl | acetyl | acetyl | acetyl | acetyl | acetyl | H | H |
| 8901 | acetyl | acetyl | acetyl | H | acetyl | acetyl | acetyl | acetyl | acetyl | acetyl | H | acetyl |
| 8902 | acetyl | acetyl | acetyl | H | acetyl | acetyl | acetyl | acetyl | acetyl | acetyl | acetyl | H |
| 8903 | acetyl | acetyl | acetyl | H | acetyl | acetyl | acetyl | acetyl | acetyl | acetyl | acetyl | acetyl |
| 8904 | acetyl | acetyl | acetyl | acetyl | H | H | H | H | H | H | H | H |
| 8905 | acetyl | acetyl | acetyl | acetyl | H | H | H | H | H | H | H | acetyl |
| 8906 | acetyl | acetyl | acetyl | acetyl | H | H | H | H | H | H | acetyl | H |
| 8907 | acetyl | acetyl | acetyl | acetyl | H | H | H | H | H | H | acetyl | acetyl |
| 8908 | acetyl | acetyl | acetyl | acetyl | H | H | H | H | H | acetyl | H | H |
| 8909 | acetyl | acetyl | acetyl | acetyl | H | H | H | H | H | acetyl | H | acetyl |
| 8910 | acetyl | acetyl | acetyl | acetyl | H | H | H | H | H | acetyl | acetyl | H |
| 8911 | acetyl | acetyl | acetyl | acetyl | H | H | H | H | H | acetyl | acetyl | acetyl |
| 8912 | acetyl | acetyl | acetyl | acetyl | H | H | H | H | acetyl | H | H | H |
| 8913 | acetyl | acetyl | acetyl | acetyl | H | H | H | H | acetyl | H | H | acetyl |
| 8914 | acetyl | acetyl | acetyl | acetyl | H | H | H | H | acetyl | H | acetyl | H |
| 8915 | acetyl | acetyl | acetyl | acetyl | H | H | H | H | acetyl | H | acetyl | acetyl |
| 8916 | acetyl | acetyl | acetyl | acetyl | H | H | H | H | acetyl | acetyl | H | H |
| 8917 | acetyl | acetyl | acetyl | acetyl | H | H | H | H | acetyl | acetyl | H | acetyl |
| 8918 | acetyl | acetyl | acetyl | acetyl | H | H | H | H | acetyl | acetyl | acetyl | H |
| 8919 | acetyl | acetyl | acetyl | acetyl | H | H | H | H | acetyl | acetyl | acetyl | acetyl |
| 8920 | acetyl | acetyl | acetyl | acetyl | H | H | H | acetyl | H | H | H | H |
| 8921 | acetyl | acetyl | acetyl | acetyl | H | H | H | acetyl | H | H | H | acetyl |
| 8922 | acetyl | acetyl | acetyl | acetyl | H | H | H | acetyl | H | H | acetyl | H |
| 8923 | acetyl | acetyl | acetyl | acetyl | H | H | H | acetyl | H | H | acetyl | acetyl |
| 8924 | acetyl | acetyl | acetyl | acetyl | H | H | H | acetyl | H | acetyl | H | H |
| 8925 | acetyl | acetyl | acetyl | acetyl | H | H | H | acetyl | H | acetyl | H | acetyl |
| 8926 | acetyl | acetyl | acetyl | acetyl | H | H | H | acetyl | H | acetyl | acetyl | H |
| 8927 | acetyl | acetyl | acetyl | acetyl | H | H | H | acetyl | H | acetyl | acetyl | acetyl |
| 8928 | acetyl | acetyl | acetyl | acetyl | H | H | H | acetyl | acetyl | H | H | H |
| 8929 | acetyl | acetyl | acetyl | acetyl | H | H | H | acetyl | acetyl | H | H | acetyl |
| 8930 | acetyl | acetyl | acetyl | acetyl | H | H | H | acetyl | acetyl | H | acetyl | H |
| 8931 | acetyl | acetyl | acetyl | acetyl | H | H | H | acetyl | acetyl | H | acetyl | acetyl |
| 8932 | acetyl | acetyl | acetyl | acetyl | H | H | H | acetyl | acetyl | acetyl | H | H |
| 8933 | acetyl | acetyl | acetyl | acetyl | H | H | H | acetyl | acetyl | acetyl | H | acetyl |
| 8934 | acetyl | acetyl | acetyl | acetyl | H | H | H | acetyl | acetyl | acetyl | acetyl | H |
| 8935 | acetyl | acetyl | acetyl | acetyl | H | H | H | acetyl | acetyl | acetyl | acetyl | acetyl |
| 8936 | acetyl | acetyl | acetyl | acetyl | H | H | acetyl | H | H | H | H | H |
| 8937 | acetyl | acetyl | acetyl | acetyl | H | H | acetyl | H | H | H | H | acetyl |
| 8938 | acetyl | acetyl | acetyl | acetyl | H | H | acetyl | H | H | H | acetyl | H |
| 8939 | acetyl | acetyl | acetyl | acetyl | H | H | acetyl | H | H | H | acetyl | acetyl |
| 8940 | acetyl | acetyl | acetyl | acetyl | H | H | acetyl | H | H | acetyl | H | H |
| 8941 | acetyl | acetyl | acetyl | acetyl | H | H | acetyl | H | H | acetyl | H | acetyl |
| 8942 | acetyl | acetyl | acetyl | acetyl | H | H | acetyl | H | H | acetyl | acetyl | H |
| 8943 | acetyl | acetyl | acetyl | acetyl | H | H | acetyl | H | H | acetyl | acetyl | acetyl |
| 8944 | acetyl | acetyl | acetyl | acetyl | H | H | acetyl | H | acetyl | H | H | H |
| 8945 | acetyl | acetyl | acetyl | acetyl | H | H | acetyl | H | acetyl | H | H | acetyl |
| 8946 | acetyl | acetyl | acetyl | acetyl | H | H | acetyl | H | acetyl | H | acetyl | H |
| 8947 | acetyl | acetyl | acetyl | acetyl | H | H | acetyl | H | acetyl | H | acetyl | acetyl |
| 8948 | acetyl | acetyl | acetyl | acetyl | H | H | acetyl | H | acetyl | acetyl | H | H |
| 8949 | acetyl | acetyl | acetyl | acetyl | H | H | acetyl | H | acetyl | acetyl | H | acetyl |
| 8950 | acetyl | acetyl | acetyl | acetyl | H | H | acetyl | H | acetyl | acetyl | acetyl | H |
| 8951 | acetyl | acetyl | acetyl | acetyl | H | H | acetyl | H | acetyl | acetyl | acetyl | acetyl |
| 8952 | acetyl | acetyl | acetyl | acetyl | H | H | acetyl | acetyl | H | H | H | H |
| 8953 | acetyl | acetyl | acetyl | acetyl | H | H | acetyl | acetyl | H | H | H | acetyl |
| 8954 | acetyl | acetyl | acetyl | acetyl | H | H | acetyl | acetyl | H | H | acetyl | H |
| 8955 | acetyl | acetyl | acetyl | acetyl | H | H | acetyl | acetyl | H | H | acetyl | acetyl |
| 8956 | acetyl | acetyl | acetyl | acetyl | H | H | acetyl | acetyl | H | acetyl | H | H |
| 8957 | acetyl | acetyl | acetyl | acetyl | H | H | acetyl | acetyl | H | acetyl | H | acetyl |
| 8958 | acetyl | acetyl | acetyl | acetyl | H | H | acetyl | acetyl | H | acetyl | acetyl | H |
| 8959 | acetyl | acetyl | acetyl | acetyl | H | H | acetyl | acetyl | H | acetyl | acetyl | acetyl |
| 8960 | acetyl | acetyl | acetyl | acetyl | H | H | acetyl | acetyl | acetyl | H | H | H |
| 8961 | acetyl | acetyl | acetyl | acetyl | H | H | acetyl | acetyl | acetyl | H | H | acetyl |
| 8962 | acetyl | acetyl | acetyl | acetyl | H | H | acetyl | acetyl | acetyl | H | acetyl | H |
| 8963 | acetyl | acetyl | acetyl | acetyl | H | H | acetyl | acetyl | acetyl | H | acetyl | acetyl |
| 8964 | acetyl | acetyl | acetyl | acetyl | H | H | acetyl | acetyl | acetyl | acetyl | H | H |
| 8965 | acetyl | acetyl | acetyl | acetyl | H | H | acetyl | acetyl | acetyl | acetyl | H | acetyl |
| 8966 | acetyl | acetyl | acetyl | acetyl | H | H | acetyl | acetyl | acetyl | acetyl | acetyl | H |
| 8967 | acetyl | acetyl | acetyl | acetyl | H | H | acetyl | acetyl | acetyl | acetyl | acetyl | acetyl |
| 8968 | acetyl | acetyl | acetyl | acetyl | H | acetyl | H | H | H | H | H | H |
| 8969 | acetyl | acetyl | acetyl | acetyl | H | acetyl | H | H | H | H | H | acetyl |
| 8970 | acetyl | acetyl | acetyl | acetyl | H | acetyl | H | H | H | H | acetyl | H |
| 8971 | acetyl | acetyl | acetyl | acetyl | H | acetyl | H | H | H | H | acetyl | acetyl |
| 8972 | acetyl | acetyl | acetyl | acetyl | H | acetyl | H | H | H | acetyl | H | H |
| 8973 | acetyl | acetyl | acetyl | acetyl | H | acetyl | H | H | H | acetyl | H | acetyl |
| 8974 | acetyl | acetyl | acetyl | acetyl | H | acetyl | H | H | H | acetyl | acetyl | H |
| 8975 | acetyl | acetyl | acetyl | acetyl | H | acetyl | H | H | H | acetyl | acetyl | acetyl |
| 8976 | acetyl | acetyl | acetyl | acetyl | H | acetyl | H | H | acetyl | H | H | H |
| 8977 | acetyl | acetyl | acetyl | acetyl | H | acetyl | H | H | acetyl | H | H | acetyl |
| 8978 | acetyl | acetyl | acetyl | acetyl | H | acetyl | H | H | acetyl | H | acetyl | H |
| 8979 | acetyl | acetyl | acetyl | acetyl | H | acetyl | H | H | acetyl | H | acetyl | acetyl |
| 8980 | acetyl | acetyl | acetyl | acetyl | H | acetyl | H | H | acetyl | acetyl | H | H |
| 8981 | acetyl | acetyl | acetyl | acetyl | H | acetyl | H | H | acetyl | acetyl | H | acetyl |
| 8982 | acetyl | acetyl | acetyl | acetyl | H | acetyl | H | H | acetyl | acetyl | acetyl | H |
| 8983 | acetyl | acetyl | acetyl | acetyl | H | acetyl | H | H | acetyl | acetyl | acetyl | acetyl |
| 8984 | acetyl | acetyl | acetyl | acetyl | H | acetyl | H | acetyl | H | H | H | H |
| 8985 | acetyl | acetyl | acetyl | acetyl | H | acetyl | H | acetyl | H | H | H | acetyl |
| 8986 | acetyl | acetyl | acetyl | acetyl | H | acetyl | H | acetyl | H | H | acetyl | H |
| 8987 | acetyl | acetyl | acetyl | acetyl | H | acetyl | H | acetyl | H | H | acetyl | acetyl |
| 8988 | acetyl | acetyl | acetyl | acetyl | H | acetyl | H | acetyl | H | acetyl | H | H |
| 8989 | acetyl | acetyl | acetyl | acetyl | H | acetyl | H | acetyl | H | acetyl | H | acetyl |
| 8990 | acetyl | acetyl | acetyl | acetyl | H | acetyl | H | acetyl | H | acetyl | acetyl | H |
| 8991 | acetyl | acetyl | acetyl | acetyl | H | acetyl | H | acetyl | H | acetyl | acetyl | acetyl |
| 8992 | acetyl | acetyl | acetyl | acetyl | H | acetyl | H | acetyl | acetyl | H | H | H |
| 8993 | acetyl | acetyl | acetyl | acetyl | H | acetyl | H | acetyl | acetyl | H | H | acetyl |
| 8994 | acetyl | acetyl | acetyl | acetyl | H | acetyl | H | acetyl | acetyl | H | acetyl | H |
| 8995 | acetyl | acetyl | acetyl | acetyl | H | acetyl | H | acetyl | acetyl | H | acetyl | acetyl |
| 8996 | acetyl | acetyl | acetyl | acetyl | H | acetyl | H | acetyl | acetyl | acetyl | H | H |
| 8997 | acetyl | acetyl | acetyl | acetyl | H | acetyl | H | acetyl | acetyl | acetyl | H | acetyl |
| 8998 | acetyl | acetyl | acetyl | acetyl | H | acetyl | H | acetyl | acetyl | acetyl | acetyl | H |
| 8999 | acetyl | acetyl | acetyl | acetyl | H | acetyl | H | acetyl | acetyl | acetyl | acetyl | acetyl |
| 9000 | acetyl | acetyl | acetyl | acetyl | H | acetyl | acetyl | H | H | H | H | H |
| 9001 | acetyl | acetyl | acetyl | acetyl | H | acetyl | acetyl | H | H | H | H | acetyl |
| 9002 | acetyl | acetyl | acetyl | acetyl | H | acetyl | acetyl | H | H | H | acetyl | H |
| 9003 | acetyl | acetyl | acetyl | acetyl | H | acetyl | acetyl | H | H | H | acetyl | acetyl |
| 9004 | acetyl | acetyl | acetyl | acetyl | H | acetyl | acetyl | H | H | acetyl | H | H |
| 9005 | acetyl | acetyl | acetyl | acetyl | H | acetyl | acetyl | H | H | acetyl | H | acetyl |
| 9006 | acetyl | acetyl | acetyl | acetyl | H | acetyl | acetyl | H | H | acetyl | acetyl | H |
| 9007 | acetyl | acetyl | acetyl | acetyl | H | acetyl | acetyl | H | H | acetyl | acetyl | acetyl |
| 9008 | acetyl | acetyl | acetyl | acetyl | H | acetyl | acetyl | H | acetyl | H | H | H |
| 9009 | acetyl | acetyl | acetyl | acetyl | H | acetyl | acetyl | H | acetyl | H | H | acetyl |
| 9010 | acetyl | acetyl | acetyl | acetyl | H | acetyl | acetyl | H | acetyl | H | acetyl | H |
| 9011 | acetyl | acetyl | acetyl | acetyl | H | acetyl | acetyl | H | acetyl | H | acetyl | acetyl |
| 9012 | acetyl | acetyl | acetyl | acetyl | H | acetyl | acetyl | H | acetyl | acetyl | H | H |
| 9013 | acetyl | acetyl | acetyl | acetyl | H | acetyl | acetyl | H | acetyl | acetyl | H | acetyl |
| 9014 | acetyl | acetyl | acetyl | acetyl | H | acetyl | acetyl | H | acetyl | acetyl | acetyl | H |
| 9015 | acetyl | acetyl | acetyl | acetyl | H | acetyl | acetyl | H | acetyl | acetyl | acetyl | acetyl |
| 9016 | acetyl | acetyl | acetyl | acetyl | H | acetyl | acetyl | acetyl | H | H | H | H |
| 9017 | acetyl | acetyl | acetyl | acetyl | H | acetyl | acetyl | acetyl | H | H | H | acetyl |
| 9018 | acetyl | acetyl | acetyl | acetyl | H | acetyl | acetyl | acetyl | H | H | acetyl | H |
| 9019 | acetyl | acetyl | acetyl | acetyl | H | acetyl | acetyl | acetyl | H | H | acetyl | acetyl |
| 9020 | acetyl | acetyl | acetyl | acetyl | H | acetyl | acetyl | acetyl | H | acetyl | H | H |
| 9021 | acetyl | acetyl | acetyl | acetyl | H | acetyl | acetyl | acetyl | H | acetyl | H | acetyl |
| 9022 | acetyl | acetyl | acetyl | acetyl | H | acetyl | acetyl | acetyl | H | acetyl | acetyl | H |
| 9023 | acetyl | acetyl | acetyl | acetyl | H | acetyl | acetyl | acetyl | H | acetyl | acetyl | acetyl |
| 9024 | acetyl | acetyl | acetyl | acetyl | H | acetyl | acetyl | acetyl | acetyl | H | H | H |
| 9025 | acetyl | acetyl | acetyl | acetyl | H | acetyl | acetyl | acetyl | acetyl | H | H | acetyl |
| 9026 | acetyl | acetyl | acetyl | acetyl | H | acetyl | acetyl | acetyl | acetyl | H | acetyl | H |
| 9027 | acetyl | acetyl | acetyl | acetyl | H | acetyl | acetyl | acetyl | acetyl | H | acetyl | acetyl |
| 9028 | acetyl | acetyl | acetyl | acetyl | H | acetyl | acetyl | acetyl | acetyl | acetyl | H | H |
| 9029 | acetyl | acetyl | acetyl | acetyl | H | acetyl | acetyl | acetyl | acetyl | acetyl | H | acetyl |
| 9030 | acetyl | acetyl | acetyl | acetyl | H | acetyl | acetyl | acetyl | acetyl | acetyl | acetyl | H |
| 9031 | acetyl | acetyl | acetyl | acetyl | H | acetyl | acetyl | acetyl | acetyl | acetyl | acetyl | acetyl |
| 9032 | acetyl | acetyl | acetyl | acetyl | acetyl | H | H | H | H | H | H | H |
| 9033 | acetyl | acetyl | acetyl | acetyl | acetyl | H | H | H | H | H | H | acetyl |
| 9034 | acetyl | acetyl | acetyl | acetyl | acetyl | H | H | H | H | H | acetyl | H |
| 9035 | acetyl | acetyl | acetyl | acetyl | acetyl | H | H | H | H | H | acetyl | acetyl |
| 9036 | acetyl | acetyl | acetyl | acetyl | acetyl | H | H | H | H | acetyl | H | H |
| 9037 | acetyl | acetyl | acetyl | acetyl | acetyl | H | H | H | H | acetyl | H | acetyl |
| 9038 | acetyl | acetyl | acetyl | acetyl | acetyl | H | H | H | H | acetyl | acetyl | H |
| 9039 | acetyl | acetyl | acetyl | acetyl | acetyl | H | H | H | H | acetyl | acetyl | acetyl |
| 9040 | acetyl | acetyl | acetyl | acetyl | acetyl | H | H | H | acetyl | H | H | H |
| 9041 | acetyl | acetyl | acetyl | acetyl | acetyl | H | H | H | acetyl | H | H | acetyl |
| 9042 | acetyl | acetyl | acetyl | acetyl | acetyl | H | H | H | acetyl | H | acetyl | H |
| 9043 | acetyl | acetyl | acetyl | acetyl | acetyl | H | H | H | acetyl | H | acetyl | acetyl |
| 9044 | acetyl | acetyl | acetyl | acetyl | acetyl | H | H | H | acetyl | acetyl | H | H |
| 9045 | acetyl | acetyl | acetyl | acetyl | acetyl | H | H | H | acetyl | acetyl | H | acetyl |
| 9046 | acetyl | acetyl | acetyl | acetyl | acetyl | H | H | H | acetyl | acetyl | acetyl | H |
| 9047 | acetyl | acetyl | acetyl | acetyl | acetyl | H | H | H | acetyl | acetyl | acetyl | acetyl |
| 9048 | acetyl | acetyl | acetyl | acetyl | acetyl | H | H | acetyl | H | H | H | H |
| 9049 | acetyl | acetyl | acetyl | acetyl | acetyl | H | H | acetyl | H | H | H | acetyl |
| 9050 | acetyl | acetyl | acetyl | acetyl | acetyl | H | H | acetyl | H | H | acetyl | H |
| 9051 | acetyl | acetyl | acetyl | acetyl | acetyl | H | H | acetyl | H | H | acetyl | acetyl |
| 9052 | acetyl | acetyl | acetyl | acetyl | acetyl | H | H | acetyl | H | acetyl | H | H |
| 9053 | acetyl | acetyl | acetyl | acetyl | acetyl | H | H | acetyl | H | acetyl | H | acetyl |
| 9054 | acetyl | acetyl | acetyl | acetyl | acetyl | H | H | acetyl | H | acetyl | acetyl | H |
| 9055 | acetyl | acetyl | acetyl | acetyl | acetyl | H | H | acetyl | H | acetyl | acetyl | acetyl |
| 9056 | acetyl | acetyl | acetyl | acetyl | acetyl | H | H | acetyl | acetyl | H | H | H |
| 9057 | acetyl | acetyl | acetyl | acetyl | acetyl | H | H | acetyl | acetyl | H | H | acetyl |
| 9058 | acetyl | acetyl | acetyl | acetyl | acetyl | H | H | acetyl | acetyl | H | acetyl | H |
| 9059 | acetyl | acetyl | acetyl | acetyl | acetyl | H | H | acetyl | acetyl | H | acetyl | acetyl |
| 9060 | acetyl | acetyl | acetyl | acetyl | acetyl | H | H | acetyl | acetyl | acetyl | H | H |
| 9061 | acetyl | acetyl | acetyl | acetyl | acetyl | H | H | acetyl | acetyl | acetyl | H | acetyl |
| 9062 | acetyl | acetyl | acetyl | acetyl | acetyl | H | H | acetyl | acetyl | acetyl | acetyl | H |
| 9063 | acetyl | acetyl | acetyl | acetyl | acetyl | H | H | acetyl | acetyl | acetyl | acetyl | acetyl |
| 9064 | acetyl | acetyl | acetyl | acetyl | acetyl | H | acetyl | H | H | H | H | H |
| 9065 | acetyl | acetyl | acetyl | acetyl | acetyl | H | acetyl | H | H | H | H | acetyl |
| 9066 | acetyl | acetyl | acetyl | acetyl | acetyl | H | acetyl | H | H | H | acetyl | H |
| 9067 | acetyl | acetyl | acetyl | acetyl | acetyl | H | acetyl | H | H | H | acetyl | acetyl |
| 9068 | acetyl | acetyl | acetyl | acetyl | acetyl | H | acetyl | H | H | acetyl | H | H |
| 9069 | acetyl | acetyl | acetyl | acetyl | acetyl | H | acetyl | H | H | acetyl | H | acetyl |
| 9070 | acetyl | acetyl | acetyl | acetyl | acetyl | H | acetyl | H | H | acetyl | acetyl | H |
| 9071 | acetyl | acetyl | acetyl | acetyl | acetyl | H | acetyl | H | H | acetyl | acetyl | acetyl |
| 9072 | acetyl | acetyl | acetyl | acetyl | acetyl | H | acetyl | H | acetyl | H | H | H |
| 9073 | acetyl | acetyl | acetyl | acetyl | acetyl | H | acetyl | H | acetyl | H | H | acetyl |
| 9074 | acetyl | acetyl | acetyl | acetyl | acetyl | H | acetyl | H | acetyl | H | acetyl | H |
| 9075 | acetyl | acetyl | acetyl | acetyl | acetyl | H | acetyl | H | acetyl | H | acetyl | acetyl |
| 9076 | acetyl | acetyl | acetyl | acetyl | acetyl | H | acetyl | H | acetyl | acetyl | H | H |
| 9077 | acetyl | acetyl | acetyl | acetyl | acetyl | H | acetyl | H | acetyl | acetyl | H | acetyl |
| 9078 | acetyl | acetyl | acetyl | acetyl | acetyl | H | acetyl | H | acetyl | acetyl | acetyl | H |
| 9079 | acetyl | acetyl | acetyl | acetyl | acetyl | H | acetyl | H | acetyl | acetyl | acetyl | acetyl |
| 9080 | acetyl | acetyl | acetyl | acetyl | acetyl | H | acetyl | acetyl | H | H | H | H |
| 9081 | acetyl | acetyl | acetyl | acetyl | acetyl | H | acetyl | acetyl | H | H | H | acetyl |
| 9082 | acetyl | acetyl | acetyl | acetyl | acetyl | H | acetyl | acetyl | H | H | acetyl | H |
| 9083 | acetyl | acetyl | acetyl | acetyl | acetyl | H | acetyl | acetyl | H | H | acetyl | acetyl |
| 9084 | acetyl | acetyl | acetyl | acetyl | acetyl | H | acetyl | acetyl | H | acetyl | H | H |
| 9085 | acetyl | acetyl | acetyl | acetyl | acetyl | H | acetyl | acetyl | H | acetyl | H | acetyl |
| 9086 | acetyl | acetyl | acetyl | acetyl | acetyl | H | acetyl | acetyl | H | acetyl | acetyl | H |
| 9087 | acetyl | acetyl | acetyl | acetyl | acetyl | H | acetyl | acetyl | H | acetyl | acetyl | acetyl |
| 9088 | acetyl | acetyl | acetyl | acetyl | acetyl | H | acetyl | acetyl | acetyl | H | H | H |
| 9089 | acetyl | acetyl | acetyl | acetyl | acetyl | H | acetyl | acetyl | acetyl | H | H | acetyl |
| 9090 | acetyl | acetyl | acetyl | acetyl | acetyl | H | acetyl | acetyl | acetyl | H | acetyl | H |
| 9091 | acetyl | acetyl | acetyl | acetyl | acetyl | H | acetyl | acetyl | acetyl | H | acetyl | acetyl |
| 9092 | acetyl | acetyl | acetyl | acetyl | acetyl | H | acetyl | acetyl | acetyl | acetyl | H | H |
| 9093 | acetyl | acetyl | acetyl | acetyl | acetyl | H | acetyl | acetyl | acetyl | acetyl | H | acetyl |
| 9094 | acetyl | acetyl | acetyl | acetyl | acetyl | H | acetyl | acetyl | acetyl | acetyl | acetyl | H |
| 9095 | acetyl | acetyl | acetyl | acetyl | acetyl | H | acetyl | acetyl | acetyl | acetyl | acetyl | acetyl |
| 9096 | acetyl | acetyl | acetyl | acetyl | acetyl | acetyl | H | H | H | H | H | H |
| 9097 | acetyl | acetyl | acetyl | acetyl | acetyl | acetyl | H | H | H | H | H | acetyl |
| 9098 | acetyl | acetyl | acetyl | acetyl | acetyl | acetyl | H | H | H | H | acetyl | H |
| 9099 | acetyl | acetyl | acetyl | acetyl | acetyl | acetyl | H | H | H | H | acetyl | acetyl |
| 9100 | acetyl | acetyl | acetyl | acetyl | acetyl | acetyl | H | H | H | acetyl | H | H |
| 9101 | acetyl | acetyl | acetyl | acetyl | acetyl | acetyl | H | H | H | acetyl | H | acetyl |
| 9102 | acetyl | acetyl | acetyl | acetyl | acetyl | acetyl | H | H | H | acetyl | acetyl | H |
| 9103 | acetyl | acetyl | acetyl | acetyl | acetyl | acetyl | H | H | H | acetyl | acetyl | acetyl |
| 9104 | acetyl | acetyl | acetyl | acetyl | acetyl | acetyl | H | H | acetyl | H | H | H |
| 9105 | acetyl | acetyl | acetyl | acetyl | acetyl | acetyl | H | H | acetyl | H | H | acetyl |
| 9106 | acetyl | acetyl | acetyl | acetyl | acetyl | acetyl | H | H | acetyl | H | acetyl | H |
| 9107 | acetyl | acetyl | acetyl | acetyl | acetyl | acetyl | H | H | acetyl | H | acetyl | acetyl |
| 9108 | acetyl | acetyl | acetyl | acetyl | acetyl | acetyl | H | H | acetyl | acetyl | H | H |
| 9109 | acetyl | acetyl | acetyl | acetyl | acetyl | acetyl | H | H | acetyl | acetyl | H | acetyl |
| 9110 | acetyl | acetyl | acetyl | acetyl | acetyl | acetyl | H | H | acetyl | acetyl | acetyl | H |
| 9111 | acetyl | acetyl | acetyl | acetyl | acetyl | acetyl | H | H | acetyl | acetyl | acetyl | acetyl |
| 9112 | acetyl | acetyl | acetyl | acetyl | acetyl | acetyl | H | acetyl | H | H | H | H |
| 9113 | acetyl | acetyl | acetyl | acetyl | acetyl | acetyl | H | acetyl | H | H | H | acetyl |
| 9114 | acetyl | acetyl | acetyl | acetyl | acetyl | acetyl | H | acetyl | H | H | acetyl | H |
| 9115 | acetyl | acetyl | acetyl | acetyl | acetyl | acetyl | H | acetyl | H | H | acetyl | acetyl |
| 9116 | acetyl | acetyl | acetyl | acetyl | acetyl | acetyl | H | acetyl | H | acetyl | H | H |
| 9117 | acetyl | acetyl | acetyl | acetyl | acetyl | acetyl | H | acetyl | H | acetyl | H | acetyl |
| 9118 | acetyl | acetyl | acetyl | acetyl | acetyl | acetyl | H | acetyl | H | acetyl | acetyl | H |
| 9119 | acetyl | acetyl | acetyl | acetyl | acetyl | acetyl | H | acetyl | H | acetyl | acetyl | acetyl |
| 9120 | acetyl | acetyl | acetyl | acetyl | acetyl | acetyl | H | acetyl | acetyl | H | H | H |
| 9121 | acetyl | acetyl | acetyl | acetyl | acetyl | acetyl | H | acetyl | acetyl | H | H | acetyl |
| 9122 | acetyl | acetyl | acetyl | acetyl | acetyl | acetyl | H | acetyl | acetyl | H | acetyl | H |
| 9123 | acetyl | acetyl | acetyl | acetyl | acetyl | acetyl | H | acetyl | acetyl | H | acetyl | acetyl |
| 9124 | acetyl | acetyl | acetyl | acetyl | acetyl | acetyl | H | acetyl | acetyl | acetyl | H | H |
| 9125 | acetyl | acetyl | acetyl | acetyl | acetyl | acetyl | H | acetyl | acetyl | acetyl | H | acetyl |
| 9126 | acetyl | acetyl | acetyl | acetyl | acetyl | acetyl | H | acetyl | acetyl | acetyl | acetyl | H |
| 9127 | acetyl | acetyl | acetyl | acetyl | acetyl | acetyl | H | acetyl | acetyl | acetyl | acetyl | acetyl |
| 9128 | acetyl | acetyl | acetyl | acetyl | acetyl | acetyl | acetyl | H | H | H | H | H |
| 9129 | acetyl | acetyl | acetyl | acetyl | acetyl | acetyl | acetyl | H | H | H | H | acetyl |
| 9130 | acetyl | acetyl | acetyl | acetyl | acetyl | acetyl | acetyl | H | H | H | acetyl | H |
| 9131 | acetyl | acetyl | acetyl | acetyl | acetyl | acetyl | acetyl | H | H | H | acetyl | acetyl |
| 9132 | acetyl | acetyl | acetyl | acetyl | acetyl | acetyl | acetyl | H | H | acetyl | H | H |
| 9133 | acetyl | acetyl | acetyl | acetyl | acetyl | acetyl | acetyl | H | H | acetyl | H | acetyl |
| 9134 | acetyl | acetyl | acetyl | acetyl | acetyl | acetyl | acetyl | H | H | acetyl | acetyl | H |
| 9135 | acetyl | acetyl | acetyl | acetyl | acetyl | acetyl | acetyl | H | H | acetyl | acetyl | acetyl |
| 9136 | acetyl | acetyl | acetyl | acetyl | acetyl | acetyl | acetyl | H | acetyl | H | H | H |
| 9137 | acetyl | acetyl | acetyl | acetyl | acetyl | acetyl | acetyl | H | acetyl | H | H | acetyl |
| 9138 | acetyl | acetyl | acetyl | acetyl | acetyl | acetyl | acetyl | H | acetyl | H | acetyl | H |
| 9139 | acetyl | acetyl | acetyl | acetyl | acetyl | acetyl | acetyl | H | acetyl | H | acetyl | acetyl |
| 9140 | acetyl | acetyl | acetyl | acetyl | acetyl | acetyl | acetyl | H | acetyl | acetyl | H | H |
| 9141 | acetyl | acetyl | acetyl | acetyl | acetyl | acetyl | acetyl | H | acetyl | acetyl | H | acetyl |
| 9142 | acetyl | acetyl | acetyl | acetyl | acetyl | acetyl | acetyl | H | acetyl | acetyl | acetyl | H |
| 9143 | acetyl | acetyl | acetyl | acetyl | acetyl | acetyl | acetyl | H | acetyl | acetyl | acetyl | acetyl |
| 9144 | acetyl | acetyl | acetyl | acetyl | acetyl | acetyl | acetyl | acetyl | H | H | H | H |
| 9145 | acetyl | acetyl | acetyl | acetyl | acetyl | acetyl | acetyl | acetyl | H | H | H | acetyl |
| 9146 | acetyl | acetyl | acetyl | acetyl | acetyl | acetyl | acetyl | acetyl | H | H | acetyl | H |
| 9147 | acetyl | acetyl | acetyl | acetyl | acetyl | acetyl | acetyl | acetyl | H | H | acetyl | acetyl |
| 9148 | acetyl | acetyl | acetyl | acetyl | acetyl | acetyl | acetyl | acetyl | H | acetyl | H | H |
| 9149 | acetyl | acetyl | acetyl | acetyl | acetyl | acetyl | acetyl | acetyl | H | acetyl | H | acetyl |
| 9150 | acetyl | acetyl | acetyl | acetyl | acetyl | acetyl | acetyl | acetyl | H | acetyl | acetyl | H |
| 9151 | acetyl | acetyl | acetyl | acetyl | acetyl | acetyl | acetyl | acetyl | H | acetyl | acetyl | acetyl |
| 9152 | acetyl | acetyl | acetyl | acetyl | acetyl | acetyl | acetyl | acetyl | acetyl | H | H | H |
| 9153 | acetyl | acetyl | acetyl | acetyl | acetyl | acetyl | acetyl | acetyl | acetyl | H | H | acetyl |
| 9154 | acetyl | acetyl | acetyl | acetyl | acetyl | acetyl | acetyl | acetyl | acetyl | H | acetyl | H |
| 9155 | acetyl | acetyl | acetyl | acetyl | acetyl | acetyl | acetyl | acetyl | acetyl | H | acetyl | acetyl |
| 9156 | acetyl | acetyl | acetyl | acetyl | acetyl | acetyl | acetyl | acetyl | acetyl | acetyl | H | H |
| 9157 | acetyl | acetyl | acetyl | acetyl | Acetyl | acetyl | acetyl | acetyl | acetyl | acetyl | H | acetyl |
| 9158 | acetyl | acetyl | acetyl | acetyl | Acetyl | acetyl | acetyl | acetyl | acetyl | acetyl | acetyl | H |

The synthetic oligosaccharide of the present invention comprises a linker X. In one embodiment, the linker X is a bond. In another embodiment the linker at one end exhibits a grouping able to enter into a covalent bonding with a reactive functional group of the carrier, e.g. an amino, thiol, or carboxyl group, and at the other end a grouping likewise able to enter into a covalent bonding with a hydroxyl group or an amino group of an oligosaccharide according to the present invention. Between the two functional groups of the linker molecule there is a biocompatible bridging molecule of suitable length, e.g. substituted or unsubstituted heteroalkylene, arylalkylene, alkylene, alkenylene, or (oligo)alkylene glycol groups. Linkers preferably comprises an substituted or unsubstituted (C₁-C₁₀)alkylene group or an substituted or unsubstituted (C₂-C₁₀)alkenylene group.

In one embodiment the linker is —(CH₂)ₚS—, where p is an integer selected from the group consisting of 3, 4, 5, 6, 7, 8, 9, 10.

In one embodiment, the present invention provides oligosaccharides 1b or 1d:

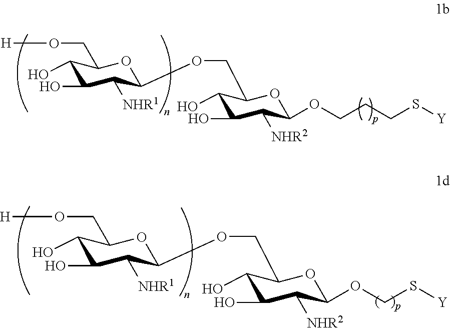

where R¹, R², n, and Y are defined as in oligosaccharide 1a; and p is an integer from 1 to 10.

Oligosaccharides 1a and 1b/d have a definite number of monosaccharide units. As indicated above, the number of monosaccharide units may be expressed by n+1. In one embodiment, n may be between about 3 and 100, preferably between about 3 and 50. In other embodiments, n is between about 5 and 25 units, about 6 and 30 units, about 6 and 24 units, and about 6 and 18 units. In another embodiment n may be 3, 4, 5, 6, 7, 8, 9, 10, 11, 12, 13, 14, 15, 16, or 17.

Oligosaccharides of the present invention may be further defined by the percentage of acetylated residues to non-acetylated residues and by the pattern (e.g. spacing) of acetylated residues relative to non-acetylated residues. For example, in oligosaccharides 1a and 1b/d the number of COCH₃ groups in R¹ and R² may be defined by z, whereby and the percentage of acetylated monosaccharide units is y, where y is z/(n+1)*100. In one embodiment, y is 90% or less, 80% or less, 70% or less, 60% or less, 50% or less, 40% or less, 30% or less, 20% or less, or 10% or less. In another embodiment, y may be 40% or less, 30% or less, 20% or less, 15% or less, 10% or less, 5% or less etc. In another embodiment, y may be at least 10%, at least 20%, at least 30%, at least 40%, at least 50%, at least 60%, at least 70%, at least 80%, at least 90%, or any combination of less than the above disclosed y values or at least equal to any of the above described y values.

In another embodiment, the oligosaccharides of the present invention may include a run of consecutive acetylated residues in one portion, a second run of consecutive non-acetylated residues in another portion, and/or singularly placed acetylated or non-acetylated residues elsewhere throughout.

The present invention essentially allows the synthesis of oligosaccharides having a specific, fixed acetylated pattern. More particularly, they may be characterized by structures in which individual acetylation positions and oligosaccharide length are engineered into a fixed, predetermined pattern. As such, the oligosaccharides of the present invention are distinguished from those produced by existing methods that result at best in heterogeneous populations of mixed sequence oligo-β-(1→6)-glucosamines in which the individual position(s) of acetylated residues is varied from one oligosaccharide to another, and where the number or percent of acetylated residues in the population is an average.

The following embodiments exemplify the oligosaccharides 1a of the present invention:

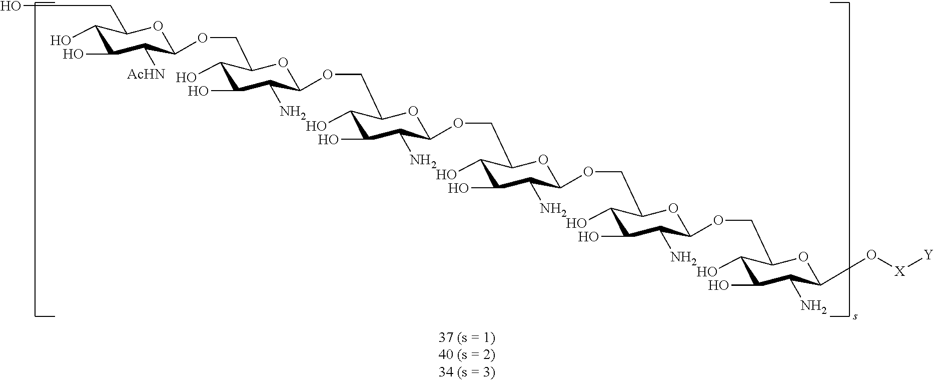

In oligosaccharides 37, 40, and 34, X and Y are as defined for oligosaccharide 1a. In particular, 37, 40, and 34 exemplify thiol oligosaccharides which may be conjugated to any carrier (Y) according to the present invention. By way of example, 37, 40, and 34 may be conjugated to form BSA conjugates such as 41, 43, and 45, respectively, or they may be conjugated to form KLH conjugates such as 42, 44, and 46, respectively.

Exemplary bacteria known or suspected to express oligo-β-(1→6)-glucosamine PNAG- and/or dPNAG structures include, but are not limited to Staphylococcus species, such as S. aureus and S. epidermidis; Escherichia coli; Yersinia species (spp.), such as Y. pestis, Y. pseudotuberculosis, and Y. entercolitica; Bordetella spp., including B. pertussis, B. bronchiseptica, and B. parapertussis; Aggregatibacter actinomycetemcomitans, Actinobacillus pleuropneumoniae; Acinetobacter spp.; Burkholderia spp.; Stenatrophomonas maltophilia, Klebsiella spp., and Shigella spp. Accordingly, specific dPNAG/PNAG oligosaccharides may be modified, depending on the specific compositional makeup, including acetylation profiles of these antigens in their respective bacterial species.

Suitable linkers comprise at one end a grouping able to enter into a covalent bonding with a reactive functional group of the carrier, e.g. an amino, thiol, or carboxyl group, and at the other end a grouping likewise able to enter into a covalent bonding with a hydroxyl group of an oligosaccharide according to the present invention. Between the two functional groups of the linker molecule there is a biocompatible bridging molecule of suitable length, e.g. substituted or unsubstituted heteroalkylene, arylalkylene, alkylene, alkenylene, or (oligo)alkylene glycol groups. Linkers preferably comprise substituted or unsubstituted (C₁-C₁₀)alkylene or (C₂-C₁₀)alkenylene groups.

If present, linkers or their respective precursors are able to react with thiol groups on the carrier are, for example, maleimide and carboxyl groups; preferred groupings able to react with aldehyde or carboxyl groups are, for example, amino or thiol groups. Preferred covalent attachments between linkers and carriers include thioethers from reaction of a thiol with an α-halo carbonyl or α-halo nitrile, including reactions of thiols with maleimide; hydrazides from reaction of a hydrazide or hydrazine with an activated carbonyl group (e.g. activated NHS-ester or acid halide); triazoles from reaction of an azide with an alkyne (e.g. via “click chemistry”); and oximes from reaction of a hydroxylamine and an aldehyde or ketone as disclosed, for example, in Lees et al., Vaccine, 24:716, 2006. Although amine-based conjugation chemistries could be used in principle for coupling linkers and/or spacers to the oligosaccharides described herein, these approaches would typically sacrifice uniformity inasmuch as the oligosaccharides of the present invention typically contain a plurality of amines bonded to second carbon of the respective monosaccharide units.

Further suitable linker molecules are known to skilled workers and commercially available or can be designed as required and depending on the functional groups present and can be prepared by known methods.

Suitable carriers are known in the art (See e.g., Remington's Pharmaceutical Sciences (18th ed., Mack Easton, Pa. (1990)) and may include, for example, proteins, peptides, lipids, polymers, dendrimers, virosomes, virus-like particles (VLPs), or combinations thereof, which by themselves may not display particular antigenic properties, but can support immunogenic reaction of a host to the oligosaccharides of the present invention (antigens) displayed at the surface of the carrier(s).

Preferably, the carrier is a protein carrier, including but are not limited to, bacterial toxoids, toxins, exotoxins, and nontoxic derivatives thereof, such as tetanus toxoid, tetanus toxin Fragment C, diphtheria toxoid, CRM (a nontoxic diphtheria toxin mutant) such as CRM 197, cholera toxoid, Staphylococcus aureus exotoxins or toxoids, Escherichia coli heat labile enterotoxin, Pseudomonas aeruginosa exotoxin A, including recombinantly produced, genetically detoxified variants thereof; bacterial outer membrane proteins, such as Neisseria meningitidis serotype B outer membrane protein complex (OMPC), outer membrane class 3 porin (rPorB) and other porins; keyhole limpet hemocyanine (KLH), hepatitis B virus core protein, thyroglobulin, albumins, such as bovine serum albumin (BSA), human serum albumin (HSA), and ovalbumin; pneumococcal surface protein A (PspA), pneumococcal adhesin protein (PsaA); purified protein derivative of tuberculin (PPD); transferrin binding proteins, polyamino acids, such as poly(lysine:glutamic acid); peptidyl agonists of TLR-5 (e.g. flagellin of motile bacteria like Listeria); and derivatives and/or combinations of the above carriers. Preferred carriers for use in humans include tetanus toxoid, CRM 197, and OMPC.

Depending on the type of bonding between the linker and the carrier, and the structural nature of the carrier and oligosaccharide, a carrier may display on average, for example, 1 to 500, 1 to 100, 1 to 20, or 3 to 9 oligosaccharide units on its surface.

Methods for attaching an oligosaccharide to a carrier, such as a carrier protein are conventional, and a skilled practitioner can create conjugates in accordance with the present invention using conventional methods. Guidance is also available in various disclosures, including, for example, U.S. Pat. Nos. 4,356,170; 4,619,828; 5,153,312; 5,422,427; and 5,445,817; and in various print and online Pierce protein cross-linking guides and catalogs (Thermo Fisher, Rockford, Ill.).

In one embodiment, the carbohydrate antigens of the present invention are conjugated to CRM 197, a commercially available protein carrier used in a number of FDA approved vaccines. CRM-conjugates have the advantage of being easier to synthesize, purify and characterize than other FDA approved carriers such as OMPC. Carbohydrate antigens may be conjugated to CRM via thiol-bromoacetyl conjugation chemistry. CRM activation may be achieved by reacting the lysine side chains with the NHS ester of bromoacetic acid using standard conditions as previously described in U.S. Pat. Appl. Publ. 2007-0134762, the disclosures of which are incorporated by reference herein. CRM may be functionalized with 10-20 bromoacetyl groups per protein (n=10-20) prior to conjugation. Conjugation may be performed at pH=9 to avoid aggregation of CRM. Careful monitoring of pH must be employed to ensure complete CRM reaction with NHS-bromoacetate while minimizing background hydrolysis of CRM. Activated CRM may be purified by size exclusion chromatography prior to conjugation. Antigen-CRM conjugates may be synthesized by reacting thiol-terminated carbohydrate antigens with bromoacetamide-activated CRM.

CRM conjugates may be purified via size exclusion chromatography to remove and recover any unreacted carbohydrate. MBTH (specific for GlcNAc residues) and Bradford assays may be used to determine carbohydrate:protein ratio and protein content, respectively, as previously described (Manzi et al., Curr. Prot. Mol. Biol., section 17.9.1 (Suppl. 32), 1995. In preferred embodiments, a minimum carbohydrate content of about 15% by weight for each conjugate may be generated. Typically, a conjugate may include about 3-20 antigens per protein carrier.

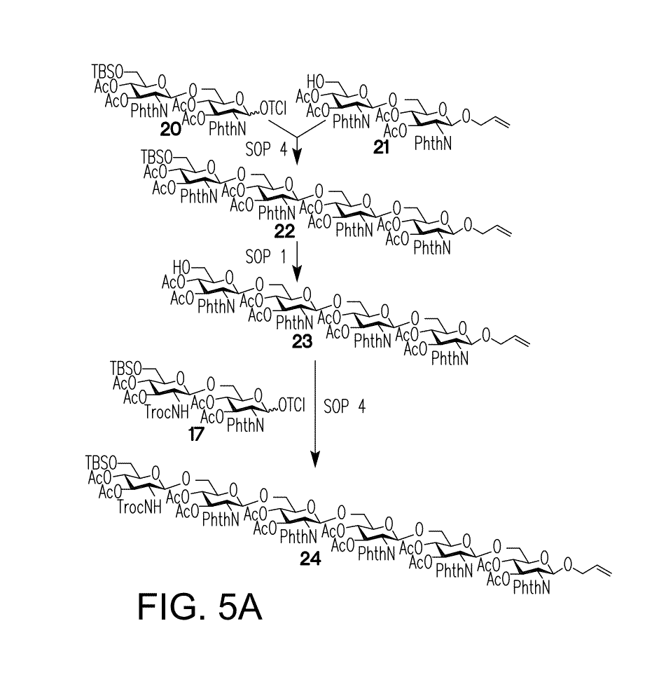

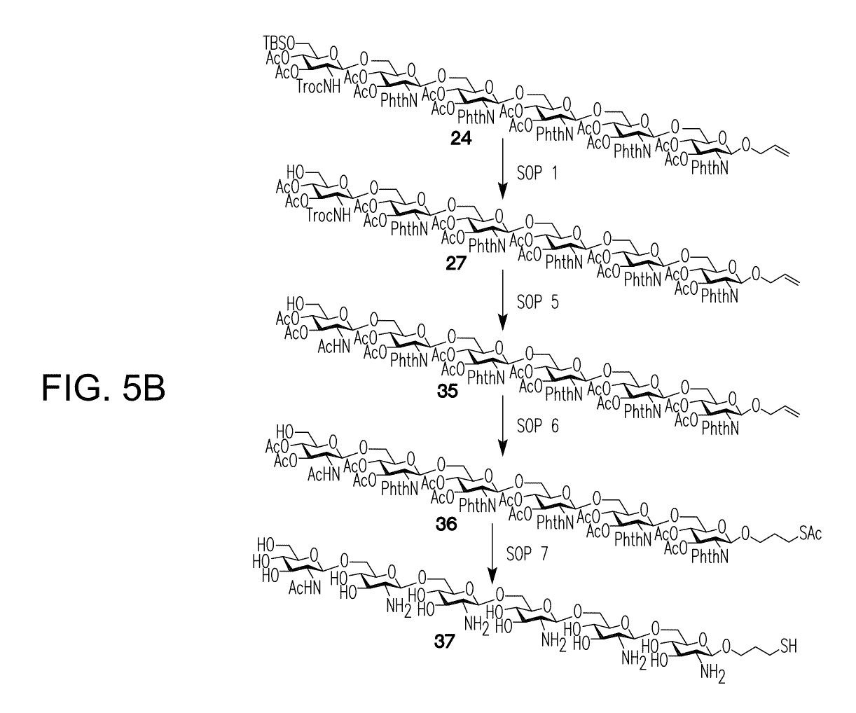

In another embodiment, oligosaccharide antigens may be conjugated to one or more carriers suitable for development of diagnostic assays, including ELISAs and microarrays. Exemplary carriers for use in such assays include bovine serum albumin (BSA), keyhole limpet hemocyanine (KLH), biotin, a label, a glass slide or a gold surface. By way of example, synthetic oligosaccharide antigens may be conjugated to BSA by a thiol-maleimide coupling procedure (FIG. 5B). Maleimide-BSA contains 15-20 maleimide groups per protein (n=15-20). Accordingly, oligosaccharide antigens may be conjugated to maleimide functionalized BSA, whereby a 20-fold molar excess of the antigen is reacted with commercially available Imject maleimide BSA (Pierce) in maleimide conjugation buffer (Pierce). Conjugation may be performed at pH=7.2 to avoid hydrolysis of the maleimide group during conjugation.

BSA conjugates may be purified via size exclusion chromatography to remove and recover any unreacted carbohydrate. Characterization via MBTH and Bradford assays may be performed along with MALDI-MS to provide information on the carbohydrate content and valency of the conjugates. In preferred embodiments, conjugates will contain a minimum carbohydrate content of about 10% by weight per BSA conjugate and >8 antigen copies per conjugate.

Methods for Synthesizing Oligo-β-(1-6)-glucosamine Structures

In another aspect, the invention provides a method for assembling mixed sequence oligo-β-(1→6)-glucosamine structures 1.

The synthetic oligosaccharides 3 according to the present invention can be synthesized by selective coupling of the donor building blocks 3a with acceptor building blocks 3b.

The structure of the donor building block 3a is shown below:

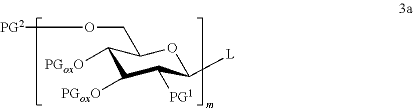

wherein the each PG¹ is independently selected from the group consisting of NPhth, NHTroc, NHTFA, NHTCA, NHCbz, and N₃;

The oxygen protecting group(s) PGₒₓ may be independently selected from the group consisting of acetyl (Ac), benzoate, trifluoro benzoate, 4-chlorobenzoate, benzyl, and 4-halobenzyl

The protecting group PG² is independently selected from the group consisting of tertbutyl dimethylsilyl (TBS), triethylsilyl (TES), tert-butyldiphenylsilyl (TBDPS), monochloracetate, trifluoroacetate, levulinoyl, 4-O-acetyl, 2,2-dimethylbutanoate, trityl, dimethoxytrityl, 9-fluoreneylmethoxycarbonyl (Fmoc), allyloxycarbonyl (AllOC) and naphthyl. Tertbutyl dimethylsilyl (TBS) and tert-butyldiphenylsilyl (TBDPS) are preferred.

The activating group L is selected from the group consisting of trichloroacetimidate (TCl), N-phenyl-trifluoroacetimidate, thioethyl, thiophenyl, thiotolyl, thioadamantyl, thioisopropyl, dibutyl phosphate, dibenzyl phosphate, and diphenylphosphate. Trichloroacetimidate (TCl) and N-phenyl-trifluoroacetimidate are preferred.

The donor building block has a defined number of monosaccharide units m. In one embodiment, m is equal or greater than 1. In another embodiment, the number of units m is between 1 and 17. In another embodiment, the number of units m is 1, 2, 3, 4, 5, 6, 7, 8, 9, 10, 11, 12, 13, 14, 15, 16, or 17. In another embodiment the number of monomeric units m is preferably 1, 2, 3, 4, 5, 6, 7, 8, 9, 10, or 11.

The structure of the acceptor building block 3b is shown below:

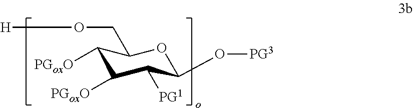

wherein in the acceptor building block 3b, each PG¹ is independently selected from the group consisting of NPhth, NHTroc, NHTFA, NHTCA, NHCbz, and N₃.

The oxygen protective groups PGₒₓ are independently selected from the group consisting of acetate, benzoate, trifluorobenzoate, 4-chlorobenzoate, benzyl, or 4-halobenzyl, 9-fluoreneylmethoxycarbonyl (Fmoc), and allyloxycarbonyl (AllOC)

The protective group PG³ is selected from the group consisting of (C₂-C₁₀)alkenyl, or (C₂-C₁₀)alkynyl. —CH₂CH═CH₂, —CH₂CCH (propargyl), and —CH₂CH₂CH₂CH═CH₂ (pent-4-enyl) are preferred.

The acceptor building block has a defined number of monosaccharide units o. In one embodiment, m is equal or greater than 1. In another embodiment, the number of units o is between 1 and 17. In another embodiment, the number of units o is 1, 2, 3, 4, 5, 6, 7, 8, 9, 10, 11, 12, 13, 14, 15, 16, or 17. In another embodiment the number of monomeric units o is preferably 1, 2, 3, 4, 5, 6, 7, 8, 9, 10, or 11.

In one embodiment m+o≧4.

To allow for the introduction of two different R¹ and/or R² groups for oligosaccharides of the present invention 1a, at least two different PG¹ groups, namely PG^{1a} and PG^{1b}, are present in the coupled oligosaccharide 3:

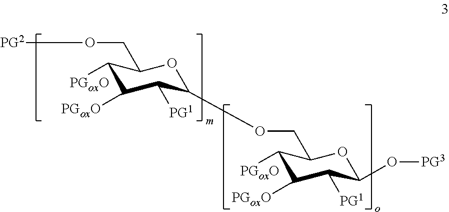

In case the coupled oligosaccharide 3 does not provide for the final number of monosaccharide units, the coupled oligomer can be converted into a corresponding acceptor block by removal of the PG² group, as shown below:

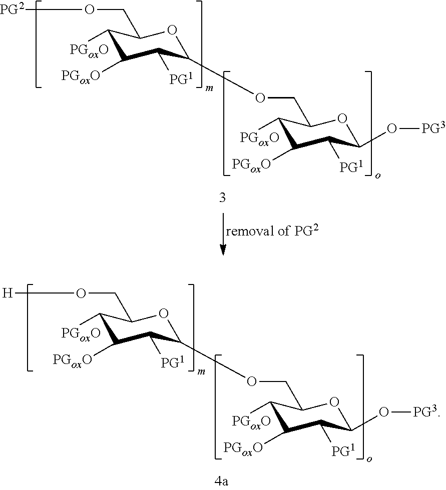

The methods for removal of the protecting group PG² are know to those skilled in the art. For example, if PG² is TBS, the TBS may be removed by treatment with a Lewis acid such as Sc(OTf)₃, and if PG² is Fmoc, the Fmoc may be removed by treatment under basic conditions such as 2,6-lutidine/triethylamine.

Alternatively, the coupled product can also be converted into a new donor building block by removal of the protecting group PG³ and introduction of the activating group L, as shown below:

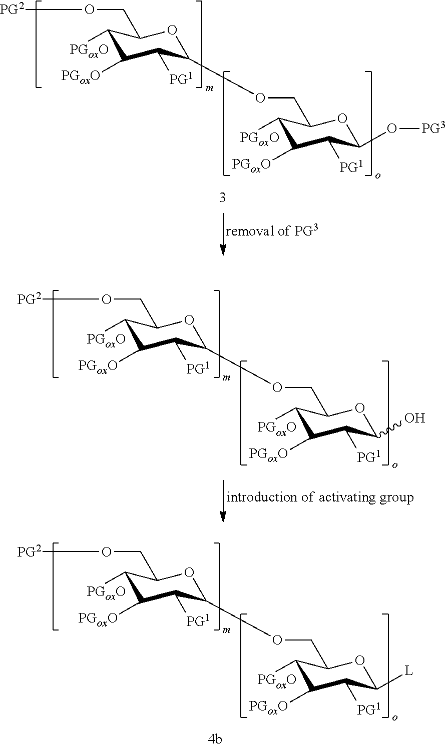

The methods for removal of the protecting group PG³ are known to those skilled in the art. For example, if PG³ is allyl, the allyl group may be removed by treatment with a catalyst such as Ir[COD(PMePh₂)]PF₆,

If the final number of monosaccharide units is not yet achieved in the product 3, the converted oligosaccharides 4a/b can be used as donor or acceptor building blocks for a further coupling reaction to build up larger oligosaccharides of the present invention.

The coupled oligosaccharide 3 can be further reacted to the oligosaccharide 1a by removal of the respective protecting groups PG^{1a/b}, PG², PG^{ox} and/or PG³.

The protecting group PG² can be removed by methods known in the art, such as the procedure outlined in SOP1 for the removal of the preferred protecting group TBS.

The coupled product 3 contains at least two different protecting groups PG¹, PG^{1a} and PG^{1b}, at least one of which is selectively removed and thus allows for a selective reaction of the deprotected group(s) while the other protecting groups may still be in place. In one embodiment, in a first step, the protecting group PG^{1a} is removed. In a preferred embodiment, the protecting group PG^{1a} is NHTroc. In one embodiment the second protecting group PG^{1b} remains in place when the first protecting group PG^{1a} is selectively removed.

In one embodiment, the deprotected N-groups are converted into a N-acetyl group. In another embodiment, the N-acetylation is performed in situ. In another embodiment, the acetylation of the deprotected N-groups is done in a separate reaction step.

The reaction conditions used for the selective removal of the protective group PG^{1a} have to be carefully chosen such that any damage of the further proceting groups in the oligosaccharide is avoided. In particular, any removal or damage of the second N-protecting group PG^{1b} has to be avoided in order to allow for a selective introduction of a substituent at the first unprotected nitrogen such as an acetyl group.

In a preferred embodiment PG^{1a} is NHTroc and PG^{1b} is NPhth. The selective removal of the Troc-protecting group is achieved by a reaction in the presence of Zinc. In a preferred embodiment the Zn is activated. Activation of the Zn can be done for example by treatment with HCl.

In case, the removal of the NHTroc group is done in situ with the N-acetylation, a mixture of Ac₂O:AcOH is present. Preferably the mixture of Ac₂O:AcOH is present in a ratio (v:v) from 10:1 to 1:10, more preferably from 5:1 to 1:5, and most preferably from 3:1 to 1:3. Typically, the reaction is performed in an ether solvent such as Et₂O, Dioxane, or THF, and preferably THF.

In one embodiment the presence of other metals than Zn is excluded in this reaction.

The protecting group PG^{1b} is removed by methods known to the person skilled in the art. In a preferred embodiment, the protecting group PG^{1b} is NPhth.

The protecting group PGₒₓ is removed by methods known to the person skilled in the art. In a preferred embodiment the protecting groups PGₒₓ are selected from the group consisting of acetate and benzoate and more preferably is acetate.

In embodiments, wherein the linker X is a bond, removal of the protecting group PG³ is sufficient. The carrier group Y may be bond to the oligosaccharide directly.

The protecting group PG³ may optionally be converted into a linker group or a precursor thereof. Alternatively, after removal of the protecting group PG³ by methods known to the person skilled in the art, a linker group may be introduced.

Suitable linker groups are substituted or unsubstituted heteroalkylene, arylalkylene, alkylene, alkenylene, or (oligo)alkylene glycol groups. Linker groups preferably comprise substituted or unsubstituted (C₁-C₁₀)alkylene moieties or an substituted or unsubstituted (C₂-C₁₀)alkenylene moieties.

In one embodiment the PG³ group is —CH₂CH═CH₂. This group may be converted into a linker —CH₂CH₂CH₂—S— by addition of thiol acetic acid under suitable conditions and optionally subsequent removal of the acetate group. Suitable reaction conditions are known to the person skilled in the art such as for example described in SOP6 (Example 7).

In a further embodiment the linker group is —(CH₂)ₚS—, where p is an integer selected from the group consisting of 3, 4, 5, 6, 7, 8, 9, 10.

In one embodiment the removal of the protecting groups PG^{1b}, PGₒₓ and optionally PG³ is done in subsequent steps, In another embodiment, the removal of the protecting groups PG^{1b}, PGₒₓ and optionally PG³ is done is performed in a single step reaction. Suitable reaction conditions for the removal of the protective groups are known to the person skilled in the art, such as exemplified in SOP7 (Example 8).

Suitable conditions for the removal of the protective groups are known to the person skilled in the art (see for example T.W. Greene, P.G.M. Wuts “Protective Groups in Organic Synthesis”, Wiley & Sons, 3^{rd} edition, 1999 herein incorporated by reference in its entirety). In Table 10 below, standard reaction conditions are listed, which may be applied to remove the respective protecting groups. However, it should be noted that in the present invention, the one N-protecting group PG^{1a} is selectively removed while at least one further N-protecting group PG^{1b} is still present in the oligosaccharide.

TABLE 10
Protecting groups and suitable removal conditions

| | | |
|---|---|---|
| Abbreviation | Full name | Removal conditions |

N-protecting groups (PG¹) - stable to conditions used to remove PG²

| | | |
|---|---|---|
| N-Phth | Phthaloyl | Hydrazine, ethylene diamine |
| NH-Troc | 2,2,2- | Zn, THF, H₂O |
| | trichloroethoxycarbonyl | |
| NH-TFA | Trifluoroacetyl | Strong base |
| NH-TCA | Trichloroacetyl | NH₃, EtOH, CHCl₃; Bu₃SnH, |
| | | AlBN |
| NH-Cbz | Benzyloxycarbonyl | H₂, Pd on Carbon |
| N3 | Azide | H₂, Pd on Carbon |

O-protecting groups (PGₒₓ independently) - stable to conditions
used to remove PG²

| | | |
|---|---|---|
| Ac | Acetyl | Hydrazine; Mild Base; |
| | | AcCl, MeOH |
| Bz | Benzoyl | Hydrazine; Mild Base |
| | Trifluorobenzoate | Hydrazine; Mild Base |
| | 4-chlorobenzoate | Hydrazine; Mild Base |
| Bn | Benzyl | H₂, Pd on Carbon |
| | 4-halobenzyl | H₂, Pd on Carbon; Pd, |
| | | amine, Lewis acid relay |

O-protecting groups (PG²)

| | | |
|---|---|---|
| TBS | tert-butyldimethylsilyl | Sc(OTf)₃, CH₃CN, H₂O; |
| | | TBAF, AcOH; HF |
| TES | Triethylsilyl | TBAF, AcOH; HF |
| TBDPS | tert-butyldiphenylsilyl | TBAF, AcOH; HF |
| MCA | Monochloracetate | NH₃, MeOH, THF |
| TFA | Trifluoroacetate | NH₃, MeOH, THF |
| Lev | Levulinoyl | Hydrazine |
| ADMB | 4-O-acetyl2,2- | DBU, MeOH |
| | dimethylbutanoate | |
| Tr | Triphenylmethyl | Mild Acid |
| DMT | Dimethoxytrityl | Mild Acid |
| FMOC | 9-fluorenylmethyl | DBU; lutidine; Et₃N, |
| | carbonate | Pyridine; Mild base |
| Alloc | Allyl oxycarbonyl | Rh(PPh₃)₃Cl; PdCl₂(PPh₃)₂; |
| | | Pd(PPh₃)₄ |
| Nap | Napthyl | DDQ; Cerium ammonium |
| | | nitrate |

Activating group (L)

| | | |
|---|---|---|
| TCl or TCA | Trichloroacetimidate | TMSOTf, TBSOTf, |
| | | BF₃—OEt₂ |
| | N-phenyl- | TMSOTf, TBSOTf, |
| | trifluoroacetimidate | BF₃—OEt₂, TfOH |
| SEt | Thioethyl | NIS, TfOH |
| SPh | Thiophenyl | NIS, TfOH |
| STol | Thiotolyl | NIS, TfOH |
| SAdm | Thioadamantyl | NIS, TfOH |
| S(iPr) | Thioisopropyl | NIS, TfOH |
| | dibutyl phosphate | TMSOTf, TBSOTf, |
| | | BF₃—OEt₂ |
| | dibenzyl phosphate | TMSOTf, TBSOTf, |
| | | BF₃—OEt₂ |
| | Diphenylphosphate | TMSOTf, TBSOTf, |
| | | BF₃—OEt₂ |

Protecting Group PG³

| | | |
|---|---|---|
| Allyl | —OCH₂CH═CH₂ | [Ir(COD)(PMePh₂)₂]PF₆, |
| | | OsO₄; PdCl₂, |
| | | acetate buffer |
| Propargyl | —OCH₂CCH | Co(CO)₈, H₂O |
| Pentenyl | —OCH₂CH₂CH₂CH═CH₂ | NBS, TfOH; NBS, H₂O |

The deprotected oligosaccharide may be further attached to a carrier group. Suitable carrier groups are described above.

In a further aspect the present invention is directed to a method of selectively converting an N-Troc group in an N-Troc protected aminosugar in the presence of at least a further protecting group into an N-acetyl group, the method comprising reacting an N-Troc protected aminosugar with a mixture of Ac₂O and AcOH in the presence of Zn.

In one embodiment, the aminosugar is selected from the group consisting of glucosamine, galactosamine, mannosamine, and fucosamine.

In one embodiment the Zn is activated. Activation of the Zn can be done for example by treatment with HCl.

In one embodiment the removal of the N-Troc group is done in situ with the N-acetylation. In this case, a mixture of Ac₂O:AcOH is present. Preferably the mixture of Ac₂O:AcOH is present in a ratio (v:v) from 10:1 to 1:10, more preferably from 5:1 to 1:5, and most preferably from 3:1 to 1:3.

Typically, the reaction is performed in an ether solvent such as Et₂O, Dioxane, or THF, and preferably THF.

In one embodiment the reaction is performed at ambient temperatures.

Building Blocks for Synthesizing Oligo-β-(1→6) Glucosamine Structures

Preferred Building blocks for the synthesis of the oligosaccharides of the present invention are described below including the four monosaccharide building blocks 6, 8, 14, and 47. The four building blocks include donor building blocks 8 and 14, and acceptor building blocks 6 and 47 below.

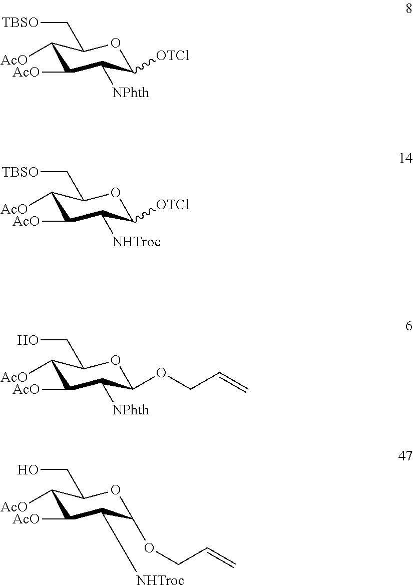

Building blocks 6 and 8 further contain an -NPhth group for selective amine group protection of individual monosaccharide units. Building blocks 14 and 47 contain a protective -NHTroc group for selective protection and subsequent acetylation of individual monosaccharide units.

Acceptor building blocks 6 and 47 have a linker precursor incorporated at the reducing end, which may be reacted with thioacetic acid and deblocked to form a conjugation-ready thiol for conjugation to a carrier as further described above. For example, when incorporated into an oligosaccharide of the present invention, the —O—CH₂—CH═CH₂ group at the reducing end may be converted into a linker comprising the sequence, —O—(CH₂)₃—SH as further described in FIGS. 5B, 6B, and 7.

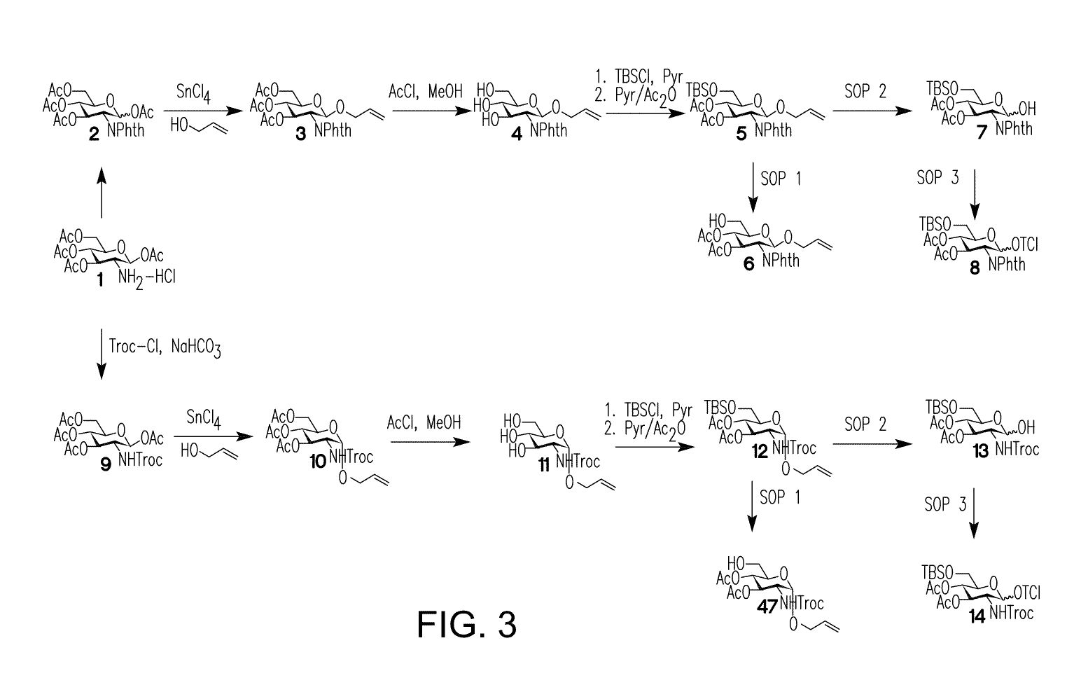

The four monosaccharide building blocks 6, 8, 14, and 47 may be synthesized from a single monosaccharide 1 as shown in FIG. 3.

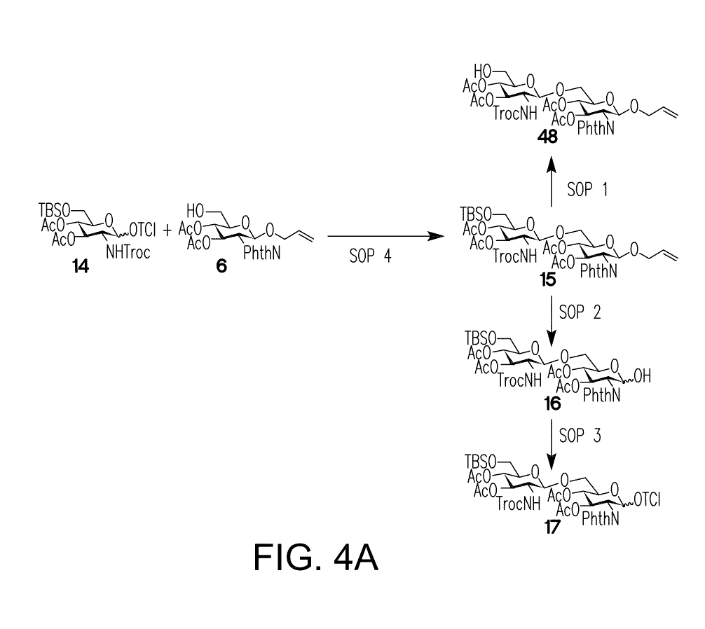

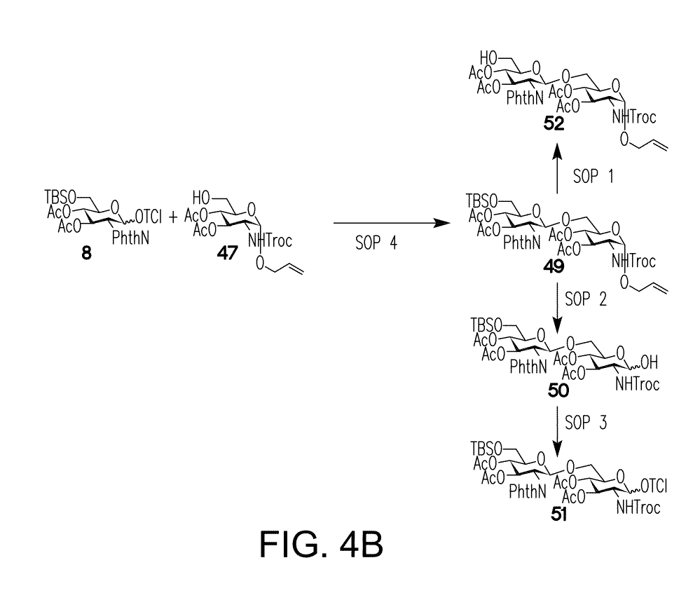

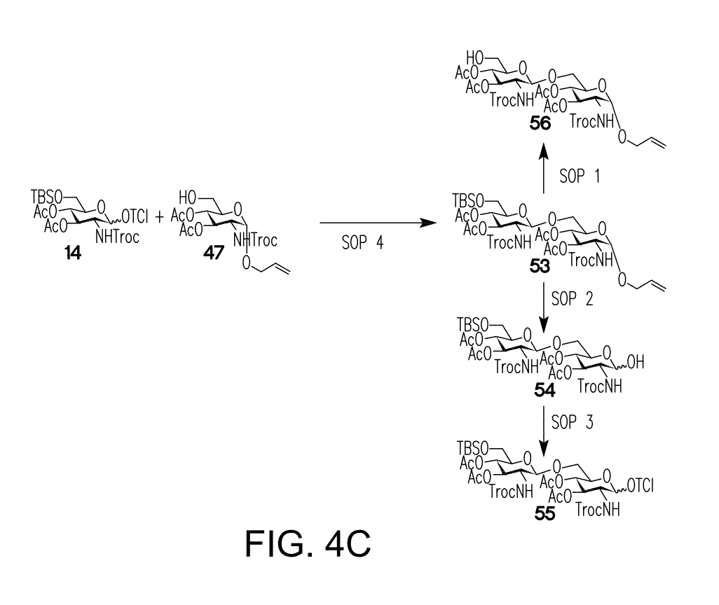

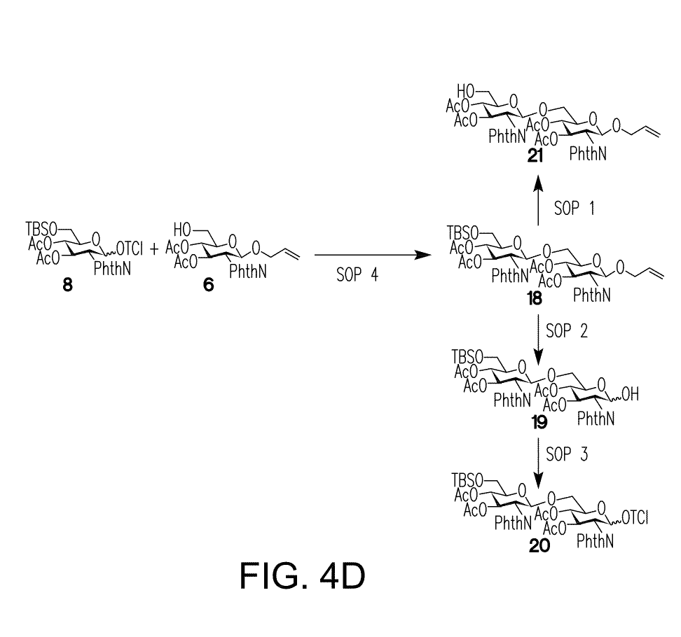

In a further aspect, the invention provides mixed disaccharide building blocks which can be used in combination with other monosaccharide- or disaccharide building blocks to form higher-order dPNAG/PNAG structures. In FIG. 4A, monosaccharide donor 14 is reacted with monosaccharide acceptor 6 to form a mixed disaccharide building block 15 in which the first monosaccharide unit is protected for selective acetylation and the amine in the second monosaccharide unit is selectively protected. In FIG. 4B, monosaccharide donor 8 is reacted with monosaccharide acceptor 47 to form a mixed disaccharide building block 49 in which the amine in the first monosaccharide unit is selectively protected and the second monosaccharide unit position is protected for selective acetylation.

In FIG. 4A, disaccharide building block 15 can be converted into a disaccharide donor 17 or a disaccharide acceptor 48 for further couplings to other acceptors or donors, respectively. Likewise, in FIG. 4B, disaccharide building block 49 can be converted into a disaccharide donor 51 or a disaccharide acceptor 52 for further couplings to other acceptors or donors, as well. For example, disaccharide donors 17 and 51 can be coupled with either of the monosaccharide acceptors 6 and 47 to form trisaccharides, or they can be coupled with disaccharide acceptors 48 and 52 to form tetrasaccharides.

In another aspect, the present invention provides disaccharide blocks for forming consecutive acetylated residues or consecutive non-acetylated resides. In FIG. 4C, monosaccharide donor 14 is reacted with monosaccharide acceptor 47 to form a disaccharide building block 53 in which each of the two monosaccharide units is protected for selective acetylation. In FIG. 4D, monosaccharide donor 8 is reacted with monosaccharide acceptor 6 to from a disaccharide building block 18 in which each of the 2-position amines in the two monosaccharide units is protected.

In FIG. 4C, disaccharide building block 53 can be converted into a disaccharide donor 55 or a disaccharide acceptor 56 for further couplings to other acceptors or donors, respectively. Likewise, in FIG. 4D, disaccharide building block 18 can be converted into a disaccharide donor 20 or a disaccharide acceptor 57 for further couplings to other acceptors or donors, as well. For example, disaccharide donors 20 and 55 can be coupled with either of the monosaccharide acceptors 6 and 47 to form trisaccharides or they can be coupled with any of the above-described disaccharide acceptors 48, 52, 56, or 57 to form tetrasaccharides.

Any of the above-described donors can be coupled to any complementary acceptor according to the method of the present invention. Accordingly, by coupling the monosaccharide-, disaccharide-, or other higher order donor modules of higher length with complementary monosaccharide-, disaccharide-, or other higher order acceptor modules of higher length, any mixed sequence oligosaccharide of the present invention can be formed in which the individual acetylation positions and oligosaccharide length are engineered into a given synthesis process in a pre-determined fashion.

Exemplary donor and acceptor building blocks for the synthesis of the oligosaccharides of the present invention are shown below:

Suitable Trisaccharide donor building blocks are

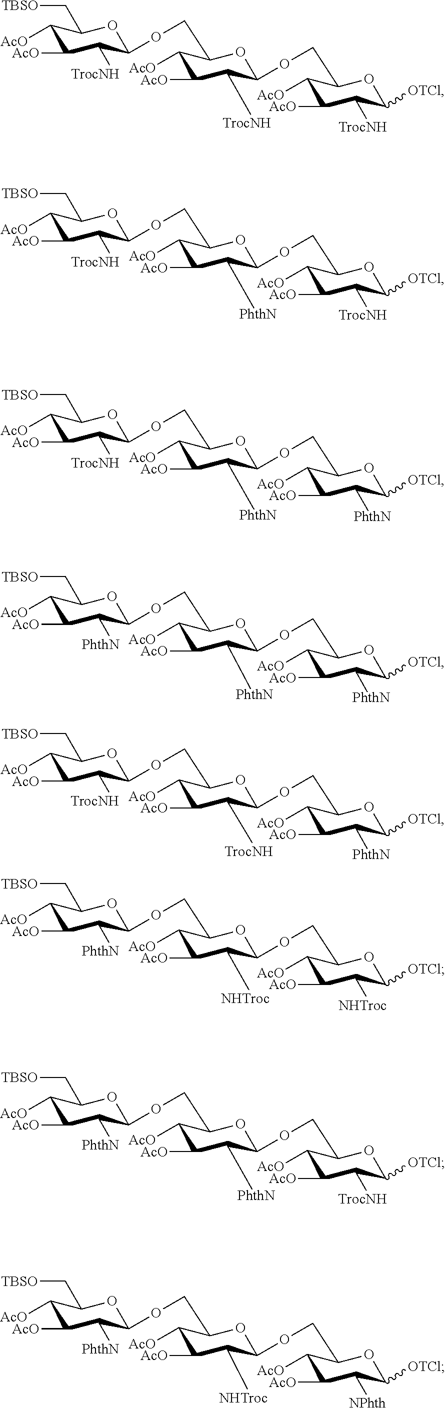

Suitable tetrasaccharide donor building blocks are

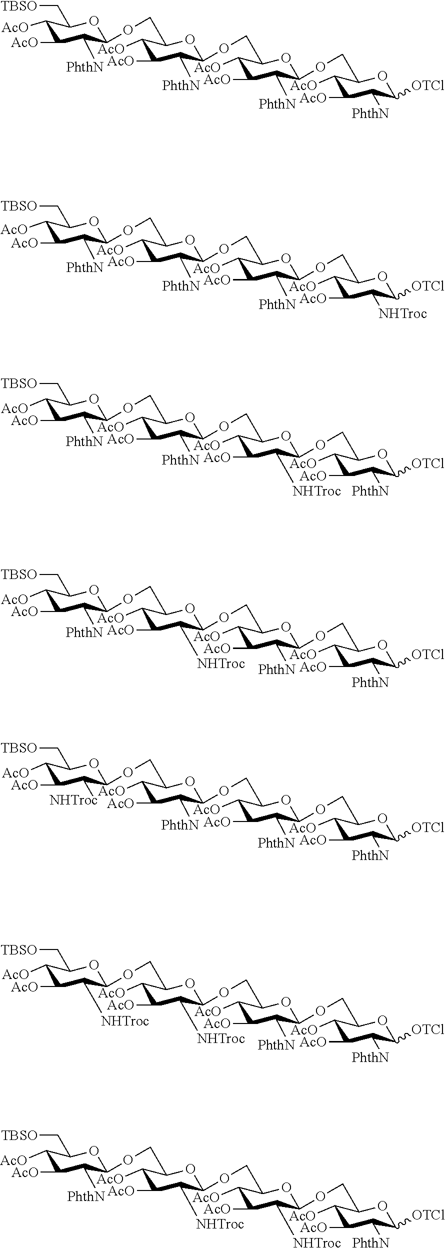

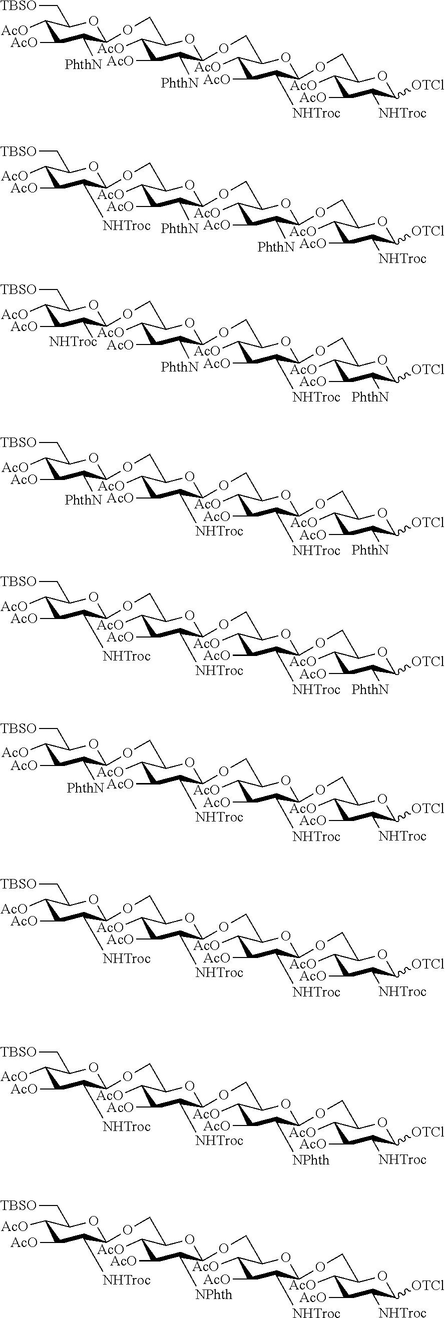

Suitable trisaccharide acceptor building blocks are:

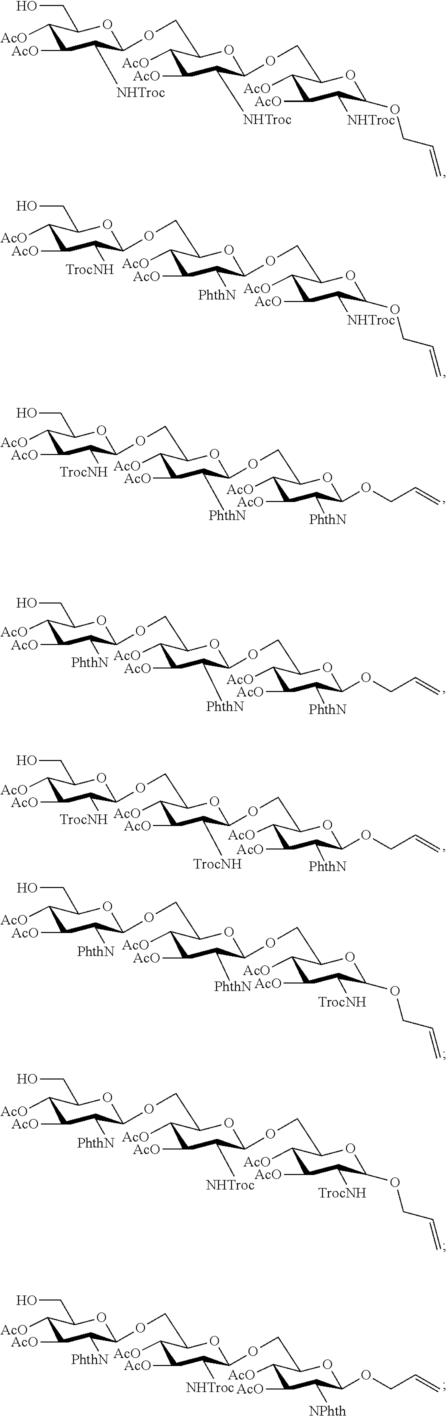

Exemplary tetramer acceptor building blocks are:

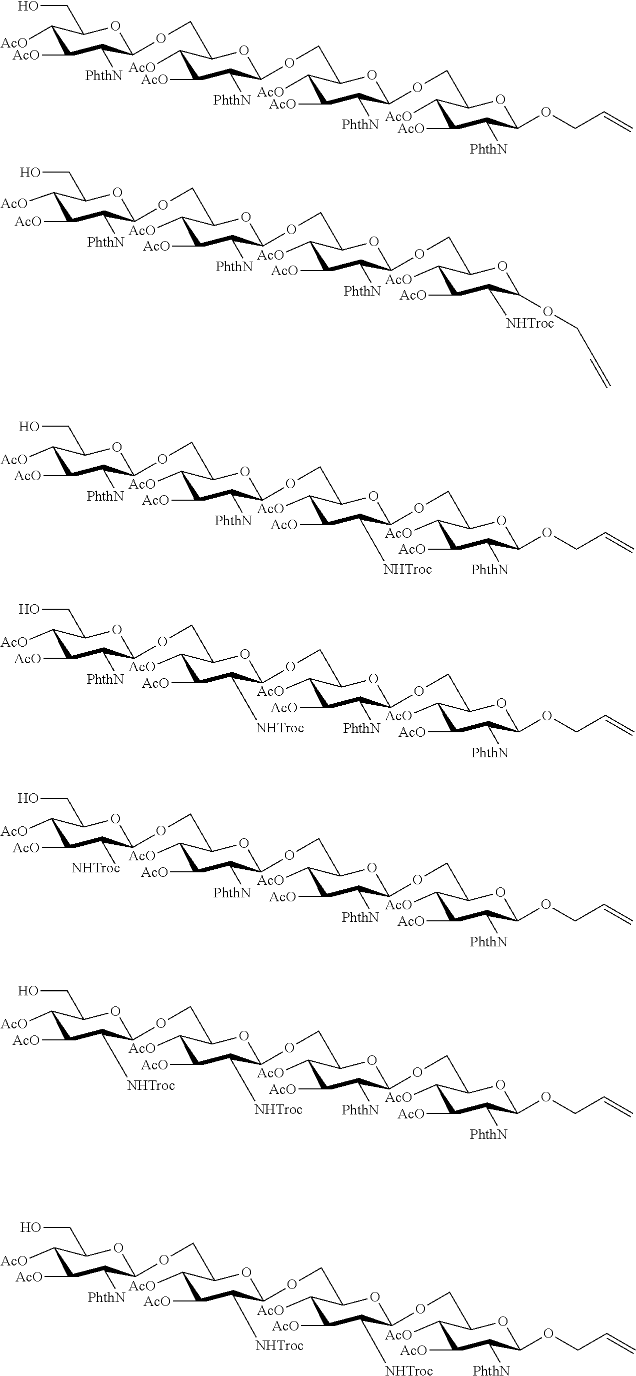

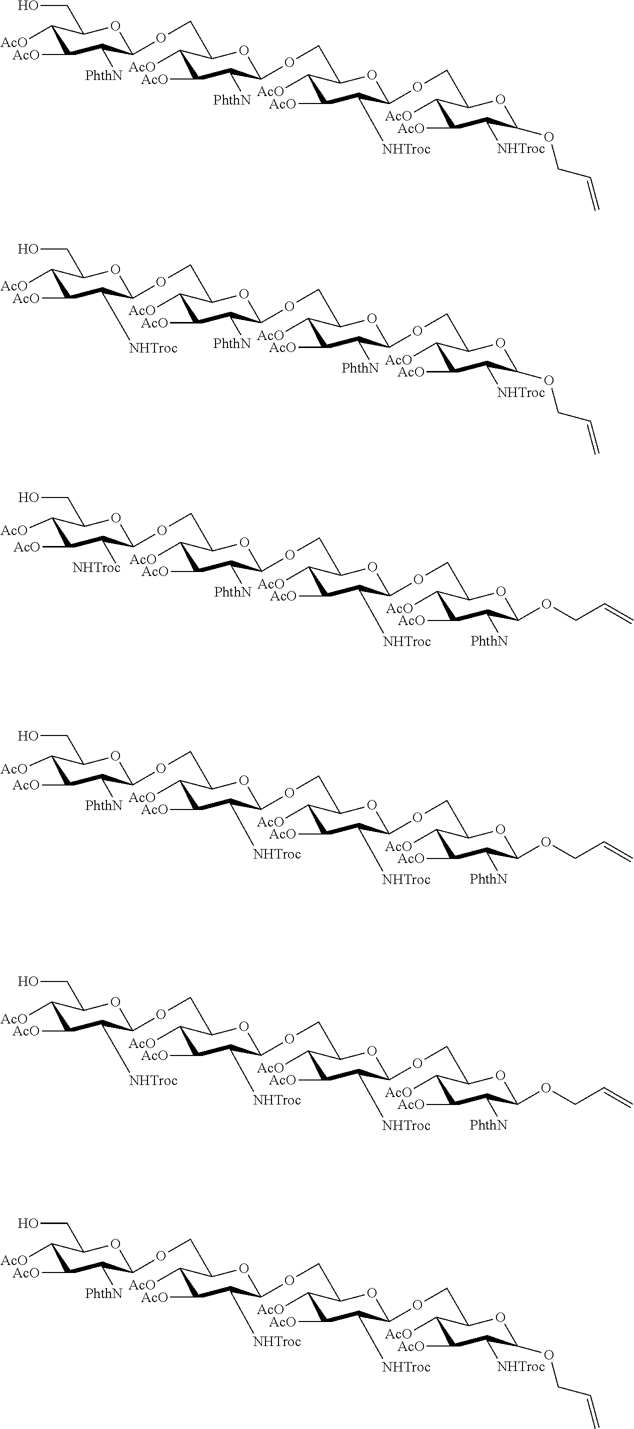

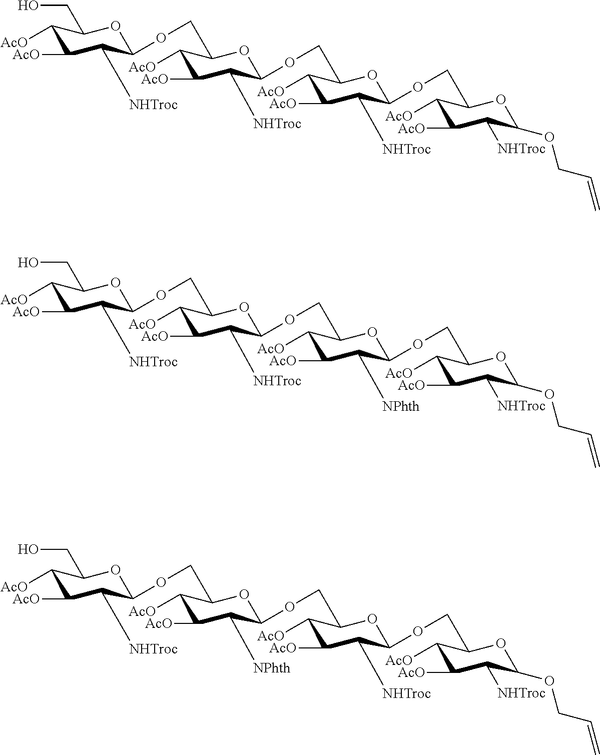

Compositions and methods for synthesizing exemplary oligosaccharides are described in the Examples below.

In FIGS. 3-10, syntheses of various oligosaccharides of the present invention proceed by a number of standard operating procedures (SOPs). In another aspect, the present invention provides a number of SOPs (or reaction steps) for synthesizing dPNAG/PNAG oligosaccharides, including SOP 1, removal of 1° TBS group(s); SOP 2, removal of allyl group(s); SOP 3, trichloroacetimidate formation; SOP 4, glycosylation using trichloroacetimidate donors; SOP 5, removal of N-Troc group and in situ N-acetylation; SOP 6, thiol addition to olefin; and SOP 7, removal of O-acetate, N-phthaloyl and S-acetate groups. SOPs 1-7 are further detailed in the Examples below.

In some cases, the above-described protecting groups may be substituted with other protecting groups customarily considered in carbohydrate chemistry, including those mentioned in “Protective Groups in Organic Synthesis”, 3.sup.rd edition, T. W. Greene and P. G. M. Wuts (Ed.), John Wiley and Sons, New York, 1999. By way of example, O-acetate groups may be replaced with O-benzoate groups for producing the antigens.

Compositions

In another aspect, the present invention provides compositions containing dPNAG/PNAG oligosaccharides 1a and a pharmaceutically acceptable vehicle. The compositions are preferably immunogenic and immunoprotective.

The present invention contemplates the use of single- and multi-valent vaccines comprising any of the synthetic oligosaccharides described herein. The identification of a single oligosaccharide antigen eliciting a protective immune response can facilitate development of a single-antigen vaccine candidate against one or more bacterial target(s) expressing dPNAG/PNAG. Thus, in one embodiment, the compositions may contain a single oligosaccharide 1a.

The present invention further contemplates multi-antigen vaccine candidates and vaccines thereof. In one embodiment, the invention provides a composition containing two, three, four or more different oligosaccharides 1a.

In another embodiment, the invention provides a composition containing two, three, four or more different oligosaccharides, including at least one oligosaccharide 1a and at least one oligosaccharide 1c:

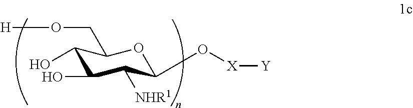

where R¹ is always H or always COCH₃; n is at least 3, X is a bond or a linker, and Y is H or a carrier (i.e., oligosaccharides 1c are fully acetylated or fully non-acetylated). When two or more oligosaccharides are used in a composition, the oligosaccharides are preferably formed separately and combined (either prior to conjugation to the carrier or after conjugation), so that the ratio of each can be controlled.

Suitable vehicles and their formulation, inclusive of other human proteins, e.g., human serum albumin, are described, for example, in Remington's Pharmaceutical Sciences (18th ed., Mack Easton Pa. (1990)). Pharmaceutically acceptable vehicles may include any vehicle that does not itself induce the production of antibodies harmful to the individual receiving the composition. Suitable vehicles may include large, slowly metabolized macromolecules such as proteins, polysaccharides, polylactic acids, polyglycolic acids, polymeric amino acids, amino acid copolymers; inactive virus particles, insoluble aluminum compounds, calcium phosphate, liposomes, virosomes, ISCOMS, microparticles, emulsions, and VLPs.

The compositions of the present invention may further include one or more adjuvants. An oligosaccharide-protein conjugate composition may further include one or more immunogenic adjuvant(s). An immunogenic adjuvant is a compound that, when combined with an antigen, increases the immune response to the antigen as compared to the response induced by the antigen alone so that less antigen can be used to achieve a similar response. For example, an adjuvant may augment humoral immune responses, cell-mediated immune responses, or both.

Those of skill in the art will appreciate that the terms “adjuvant,” and “carrier,” can overlap to a significant extent. For example, a substance which acts as an “adjuvant” may also be a “carrier,” and certain other substances normally thought of as “carriers,” for example, may also function as an “adjuvant.” Accordingly, a substance which may increase the immunogenicity of the synthetic oligosaccharide or carrier associated therewith is a potential adjuvant. As used herein, a carrier is generally used in the context of a more directed site-specific conjugation to an oligosaccharide of the present invention, whereby an adjuvant is generally used in a less specific or more generalized structural association therewith.

Exemplary adjuvants and/or adjuvant combinations may be selected from the group consisting of mineral salts, including aluminum salts, such as aluminum phosphate and aluminum hydroxide (alum) (e.g., Alhydrogel™, Superfos, Denmark) and calcium phosphate; RIBI, which contains three components extracted from bacteria, monophosphoryl lipid A, trehalose dimycolate, and cell wall skeleton (MPL+TDM+CWS) in a 2% squalene/Tween 80 emulsion, whereby any of the 3 components MPL, TDM or CWS may also be used alone or combined 2 by 2; toll-like receptor (TLR) agonists, including, for example, agonists of TLR-1 (e.g. tri-acyl lipopeptides); agonists of TLR-2 [e.g. peptidoglycan of gram-positive bacteria like streptococci and staphylococci; lipoteichoic acid]; agonists of TLR-3 (e.g. double-stranded RNA and their analogs such as poly 1:C); agonists of TLR-4 (e.g. lipopolysaccharide (endotoxin) of gram-negative bacteria like Salmonella and E. coli); agonists of TLR-5 (e.g. flagellin of motile bacteria like Listeria); agonists of TLR-6 (e.g. with TLR-2 peptidoglycan and certain lipids (diacyl lipopeptides)); agonists of TLR-7 (e.g. single-stranded RNA (ssRNA) genomes of such viruses as influenza, measles, and mumps; and small synthetic guanosine-base antiviral molecules like Ioxoribine and ssRNA and their analogs); agonists of TLR-8 (e.g. binds ssRNA); agonists of TLR-9 (e.g. unmethylated CpG of the DNA of the pathogen and their analogs; agonists of TLR-10 (function not defined) and TLR-11—(e.g. binds proteins expressed by several infectious protozoans (Apicomplexa), specific toll-like receptor agonists include monophosphoryl lipid A (MPL®), 3 De-O-acylated monophosphoryl lipid A (3 D-MPL), OM-174 (E. coli lipid A derivative); OM triacyl lipid A derivative, and other MPL- or lipid A-based formulations and combinations thereof, including MPC-SE, RC-529 (Dynavax Technologies), AS01 (liposomes+MPL+QS21), AS02 (oil-in-water PL+QS-21), and AS04 (Alum+MPL)(GlaxoSmith Kline, Pa.), CpG-oligodeoxynucleotides (ODNs) containing immunostimulatory CpG motifs, double-stranded RNA, polyinosinic:polycytidylic acid (poly I:C), and other oligonucleotides or polynucleotides optionally encapsulated in liposomes; oil-in-water emulsions, including AS03 (GlaxoSmith Kline, Pa.), MF-59 (microfluidized detergent stabilized squalene oil-in-water emulsion; Novartis), and Montanide ISA-51 VG (stabilized water-in-oil emulsion) and Montanide ISA-720 (stabilized water/squalene; Seppic Pharmaceuticals, Fairfield, N.J.); cholera toxin B subunit; saponins, such as Quil A or QS21, an HPLC purified non-toxic fraction derived from the bark of Quillaja Saponaria Molina (STIMULON™ (Antigenics, Inc., Lexington, Mass.) and saponin-based adjuvants, including immunostimulating complexes (ISCOMs; structured complex of saponins and lipids) and other ISCOM-based adjuvants, such as ISCOMATRIX™ and AbISCO®-100 and -300 series adjuvants (Isconova AB, Uppsala, Sweden); QS21 and 3 D-MPL together with an oil in water emulsion as disclosed in U.S. Pat. Appl. No. 2006/0073171; stearyl tyrosine (ST) and amide analogs thereof; virus-like particles (VLPs) and reconstituted influenza virosomes (IRIVs); complete Freund's adjuvant (CFA); incomplete Freund's adjuvant (IFA); E. coli heat-labile enterotoxin (LT); immune-adjuvants, including cytokines, such as IL-2, IL-12, GM-CSF, Flt3, accessory molecules, such as B7.1, and mast cell (MC) activators, such as mast cell activator compound 48/80 (C48/80); water-insoluble inorganic salts; liposomes, including those made from DNPC/Chol and DC Chol; micelles; squalene; squalane; muramyl dipeptides, such as N-acetyl-muramyl-L-threonyl-D-isoglutamine (thr-MDP) as found in U.S. Pat. No. 4,606,918, N-acetyl-normuramyl-L-alanyl-D-isoglutamine (nor-MDP), and N-acetylmuramyl-L-alanyl-D-isoglutaminyl-L-alanine-2-(1′2′-dipalmitoyl-n-glycero-3-hydroxyphosphoryl; SAF-1 (Syntex); AS05 (GlaxoSmith Kline, Pa.); and combinations thereof.

In preferred embodiments, adjuvant potency may be enhanced by combining multiple adjuvants as described above, including combining various delivery systems with immunopotentiating substances to form multi-component adjuvants with the potential to act synergistically to enhance antigen-specific immune responses in vivo. Exemplary immunopotentiating substances include the above-described adjuvants, including, for example, MPL and synthetic derivatives, MDP and derivatives, oligonucleotides (CpG etc), ds RNAs, alternative pathogen-associated molecular patterns (PAMPs)(E. coli heat labile enterotoxin; flagellin, saponins (QS-21 etc), small molecule immune potentiators (SMIPs, e.g., resiquimod [R848]), cytokines, and chemokines.

Methods of Treating or Preventing Staphyloccus Infections

Oligosaccharide Compositions

In one embodiment, the present invention provides pharmaceutically acceptable immunogenic or immunoprotective oligosaccharide compositions and their use in methods for preventing Staphylococcus infection in a patient in need thereof. In one embodiment, comprising administering an effective amount of an oligosaccharide of the present invention. An immunogenic or immunoprotective composition will include a “sufficient amount” or “an immunologically effective amount” of a dPNAG/PNAG-protein conjugate according to the present invention, as well as any of the above mentioned components, for purposes of generating an immune response or providing protective immunity, as further defined herein.

Administration of the oligosaccharide- or oligosaccharide conjugate compositions or antibodies, as described herein may be carried out by any suitable means, including by parenteral administration (e.g., intravenously, subcutaneously, intradermally, or intramuscularly); by topical administration, of for example, antibodies to an airway surface; by oral administration; by in ovo injection in birds, for example, and the like. Preferably, they are administered intramuscularly.

Typically, the compositions may be prepared as injectables, either as liquid solutions or suspensions; solid forms suitable for solution in, or suspension in, liquid vehicles prior to injection. An aqueous composition for parenteral administration, for example, may include a solution of the immunogenic component(s) dissolved or suspended in a pharmaceutically acceptable vehicle or diluent, preferably a primarily aqueous vehicle. An aqueous composition may be formulated as a sterile, pyrogen-free buffered saline or phosphate-containing solution, which may include a preservative or may be preservative free. Suitable preservatives include benzyl alcohol, parabens, thimerosal, chlorobutanol, and benzalkonium chloride, for example. Aqueous solutions are preferably approximately isotonic, and its tonicity may be adjusted with agents such as sodium tartrate, sodium chloride, propylene glycol, and sodium phosphate. Additionally, auxiliary substances required to approximate physiological conditions, including pH adjusting and buffering agents, tonicity adjusting agents, wetting or emulsifying agents, pH buffering substances, and the like, including sodium acetate, sodium lactate, sodium chloride, potassium chloride, calcium chloride, sorbitan monolaurate, triethanolamine oleate, etc. may be included with the vehicles described herein.

Compositions may be formulated in a solid or liquid form for oral delivery. For solid compositions, nontoxic and/or pharmaceutically acceptable solid vehicles may include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition may be formed by incorporating any of the normally employed excipients, including those vehicles previously listed, and a unit dosage of an active ingredient, that is, one or more compounds of the invention, whether conjugated to a carrier or not. Topical application of antibodies to an airway surface can be carried out by intranasal administration (e.g., by use of dropper, swab, or inhaler which deposits a pharmaceutical formulation intranasally). Topical application of the antibodies to an airway surface can also be carried out by inhalation administration, such as by creating respirable particles of a pharmaceutical formulation (including both solid particles and liquid particles) containing the antibodies as an aerosol suspension, and then causing the subject to inhale the respirable particles. Methods and apparatuses for administering respirable particles of pharmaceutical formulations are well known, and any conventional technique can be employed. Oral administration may be in the form of an ingestable liquid or solid formulation.

The preparation of such pharmaceutical compositions is within the ordinary skill in the art, and may be guided by standard reference books such as Remington the Science and Practice of Pharmacy, Lippincott Williams & Wilkins; 21 ed., May 1, 2005, which is incorporated herein by reference.

The concentration of the oligosaccharides of the invention in the pharmaceutical formulations can vary widely, i.e., from less than about 0.1%, usually at or at least about 0.1% to as much as 20% to 50% or more by weight, and may be selected on the basis of fluid volumes, viscosities, stability, etc., and/or in accordance with the particular mode of administration selected. A human unit dose form of the compounds and composition is typically included in a pharmaceutical composition that comprises a human unit dose of an acceptable vehicle, preferably an aqueous vehicle, and is administered in a volume of fluid that is known by those of skill in the art to be used for administration of such compositions to humans, and is adjusted according to commonly understood principles for a particular subject to be treated. Thus in one embodiment, the invention provides a unit dosage of the vaccine components of the invention in a suitable amount of an aqueous solution, such as 0.1-3 ml, preferably 0.2-2 mL.

The compositions of the present invention may be administered to any animal species at risk for developing an infection by a microbial species expressing a PNAG and/or PNAG antigen.

The present invention can also be used to treat or prevent other bacteria infections where the bacterium is known or suspected to express PNAG or dPNAG. Suitable bacteria that can be treated with the present invention include Staphylococcus species, such as S. aureus and S. epidermidis; Escherichia coli; Yersinia species (spp.), such as Y. pestis, Y. pseudotuberculosis, and Y. entercolitica; Bordetella spp., including B. pertussis, B. bronchiseptica, and B. parapertussis; Aggregatibacter actinomycetemcomitans, Actinobacillus pleuropneumoniae; Acinetobacter spp.; Burkholderia spp.; Stenatrophomonas maltophilia, Klebsiella spp., and Shigella spp. Accordingly, specific dPNAG/PNAG oligosaccharides may be modified, depending on the specific compositional makeup, including acetylation profiles of these antigens in their respective bacterial species.

The treatment may be given in a single dose schedule, or preferably a multiple dose schedule in which a primary course of treatment may be with 1-10 separate doses, followed by other doses given at subsequent time intervals required to maintain and or reinforce the response, for example, at 1-4 months for a second dose, and if needed, a subsequent dose(s) after several months. Examples of suitable treatment schedules include: (i) 0, 1 month and 6 months, (ii) 0, 7 days and 1 month, (iii) 0 and 1 month, (iv) 0 and 6 months, or other schedules sufficient to elicit the desired responses expected to reduce disease symptoms, or reduce severity of disease.

The amounts effective for inducing an immune response or providing protective immunity will depend on a variety of factors, including the oligosaccharide composition, conjugation to a carrier, inclusion and nature of adjuvant(s), the manner of administration, the weight and general state of health of the patient, and the judgment of the prescribing physician. By way of example, the amounts may generally range for the initial immunization (that is for a prophylactic administration) from about 1.0 μg to about 5,000 μg of oligosaccharide for a 70 kg patient, (e.g., 1.0 μg, 2.0 μg, 2.5 μg, 3.0 μg, 3.5 μg, 4.0 μg, 4.5 μg, 5.0 μg, 7.5 μg, 10 μg, 12.5 μg, 15 μg, 17.5 μg, 20 μg, 25 μg, 30 μg, 35 μg, 40 μg, 45 μg, 50 μg, 75 μg, 100 μg, 250 μg, 500 μg, 750 μg, 1,000 μg, 1,500 μg, 2,000 μg, 2,500 μg, 3,000 μg, 3,500 μg, 4,000 μg, 4,500 μg or 5,000 μg). The actual dose administered to a subject is often, but not necessarily, determined according to an appropriate amount per kg of the subject's body weight. For example, an effective amount may be about 0.1 μg to 5 μg/kg body weight.

A primary dose may optionally be followed by boosting dosages of from about 1.0 to about 1,000 of peptide (e.g., 1.0 μg, 2.0 μg, 2.5 μg, 3.0 μg, 3.5 μg, 4.0 μg, 4.5 μg, 5.0 μg, 7.5 μg, 10 μg, 12.5 μg, 15 μg, 17.5 μg, 20 μg, 25 μg, 30 μg, 35 μg, 40 μg, 45 μg, 50 μg, 75 μg, 100 μg, 250 μg, 500 μg, 750 μg, 1,000 μg, 1,500 μg, 2,000 μg, 2,500 μg, 3,000 μg, 3,500 μg, 4,000 μg, 4,500 μg or 5,000 μg) pursuant to a boosting regimen over weeks to months depending upon the patient's response and condition by measuring specific T cell activity in the patient's blood.

The immunogenic compositions comprising a compound of the invention may be suitable for use in adult humans or in children, including young children or others at risk for contracting an infection caused by a dPNAG/PNAG-expressing bacterial species. Optionally such a composition may be administered in combination with other pharmaceutically active substances, and frequently it will be administered in combination with other vaccines as part of a childhood vaccination program.

Antibody Compositions

In another embodiment, the invention provides an antibody preparation against one or more oligo-β-(1→6)-glucosamine 1a in accordance with the present invention. The antibody preparation may include any member from the group consisting of polyclonal antibody, monoclonal antibody, mouse monoclonal IgG antibody, humanized antibody, chimeric antibody, fragment thereof, or combination thereof.

Pharmaceutical antibody compositions may be used in a method for providing passive immunity against a bacterial target species of interest, including S. aureus and other dPNAG/PNAG-expressing bacteria. A pharmaceutical antibody composition may be administered to an animal subject, preferably a human, in an amount sufficient to prevent or attenuate the severity, extent of duration of the infection by the bacterial target species of interest.

The administration of the antibody may be either prophylactic (prior to anticipated exposure to a bacterial infection) or therapeutic (after the initiation of the infection, at or shortly after the onset of the symptoms). The dosage of the antibodies will vary depending upon factors as the subject's age, weight and species. In general, the dosage of the antibody may be in a range from about 1-10 mg/kg body weight. In a preferred embodiment, the antibody is a humanized antibody of the IgG or the IgA class. The route of administration of the antibody may be oral or systemic, for example, subcutaneous, intramuscular or intravenous.

Antibodies in Diagnostic Assays

In a further aspect, the present invention provides compositions and methods for inducing production of antibodies for diagnosing, treating, and/or preventing one or more infections caused by dPNAG/PNAG expressing bacteria.

Antisera to dPNAG/PNAG conjugates may be generated in New Zealand white rabbits by 3-4 subcutaneous injections over 13 weeks. A pre-immune bleed may generate about 5 mL of baseline serum from each rabbit. A prime injection (10 μg antigen equivalent) may be administered as an emulsion in complete Freund's adjuvant (CFA). Subsequent injections (5 μg antigen equivalent) may be given at three week intervals in incomplete Freund's adjuvant (IFA). Rabbits may be bled every two weeks commencing one week after the third immunization.

Approximately 25-30 mL of serum per rabbit may be generated from each bleeding event and frozen at −80° C. Serum may be analyzed by ELISA against the corresponding dPNAG/PNAG conjugate as described below. In addition, antisera from later bleeds may be affinity purified as further described below.

The oligosaccharides and antibodies generated therefrom can be used as diagnostic reagents for detecting dPNAG-PNAG structures or antibodies thereagainst, which are present in biological samples. The detection reagents may be used in a variety of immunodiagnostic techniques, known to those of skill in the art, including ELISA- and microarray-related technologies. In addition, these reagents may be used to evaluate antibody responses, including serum antibody levels, to immunogenic oligosaccharide conjugates. The assay methodologies of the invention typically involve the use of labels such as fluorescent, chemiluminescent, radioactive, enzymatic labels or dye molecules, and/or secondary immunologic reagents for direct or indirect detection of a complex between an antigen or antibody in a biological sample and a corresponding antibody or antigen bound to a solid support.

Such assays typically involve separation of unbound antibody in a liquid phase from a solid phase support to which antibody-antigen complexes are bound. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

Typically, a solid support is first reacted with a first binding component (e.g., an anti-dPNAG-PNAG antibody or dPNAG-PNAG oligosaccharide) under suitable binding conditions such that the first binding component is sufficiently immobilized to the support. In some cases, mobilization to the support can be enhanced by first coupling the antibody or oligosaccharide to a protein with better binding properties, or that provides for immobilization of the antibody or antigen on the support without significant loss of antibody binding activity or specificity. Suitable coupling proteins include, but are not limited to, macromolecules such as serum albumins including bovine serum albumin (BSA), keyhole limpet hemocyanin (KLH), immunoglobulin molecules, thyroglobulin, ovalbumin, and other proteins well known to those skilled in the art. Other molecules that can be used to bind antibodies the support include polysaccharides, polylactic acids, polyglycolic acids, polymeric amino acids, amino acid copolymers, and the like. Such molecules and methods of coupling these molecules are well known to those of ordinary skill in the art and are described in, for example, U.S. Pat. No. 7,595,307, U.S. Pat. Appl. No. US 2009/0155299, the disclosures and cited references therein of which are incorporated by reference herein.

The following examples are included for purposes of illustration and are not intended to limit the scope of the invention.

### EXAMPLES

### Example 1

### Generation of dPNAG/PNAG Test Libraries

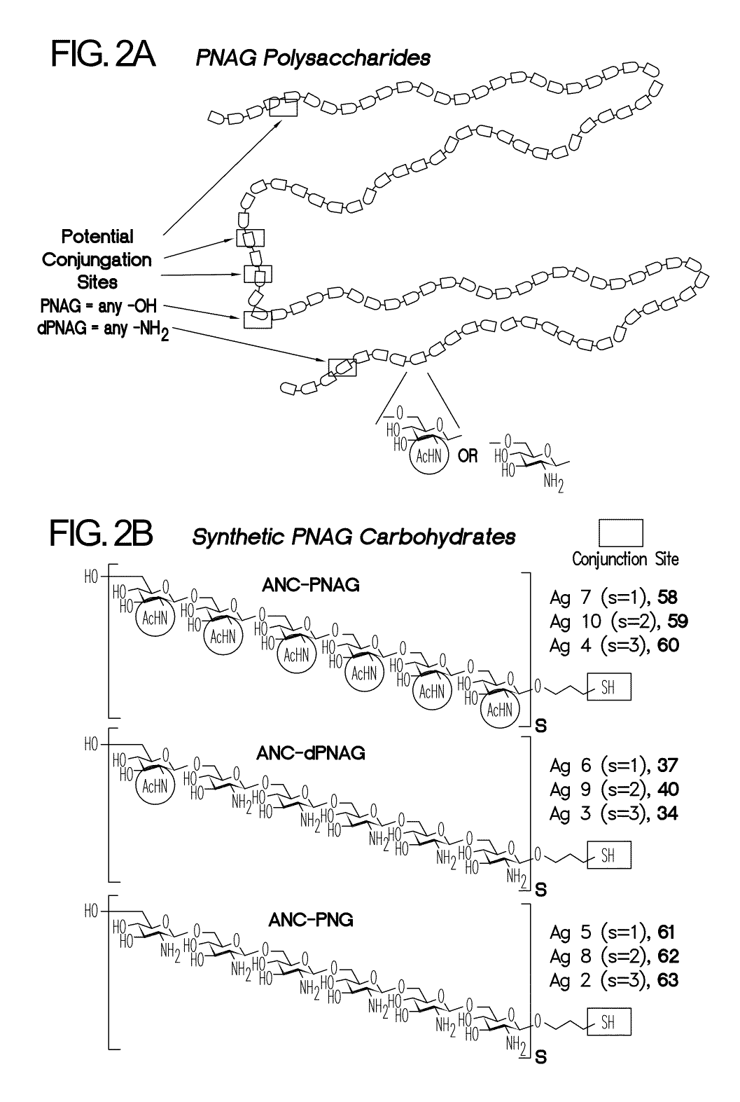

The above-described monosaccharide- and disaccharide building blocks were used in higher antigen assembly to initially provide a route for forming three large, defined poly-glucosamine structures. Thus, an initial library was generated from three basic hexamer units or “analog cores” (FIG. 2B) corresponding to: (1) a fully acetylated N-acetyl PNAG (ANC-PNAG; exemplified by thiol oligosaccharide 58), (2) a partially acetylated (16.7%) PNAG (ANC-dPNAG exemplified by thiol oligosaccharide 37) and (3) a fully non-acetylated PNAG (ANC-PNG; exemplified by thiol oligosaccharide 61).

Molecules generated with the first analog core in the initial PNAG library represented by 58, 59, 60 can serve as a control group and to mimic functionally the major secreted component of staphylococcal biofilm.

The second analog core containing a partially acetylated PNAG molecule exemplified by oligosaccharides 37, 40, 34, and conjugates thereof (see FIG. 2B) represents one possible product of chemical or ica B-mediated deacetylation (16.7% N-acetyl), which was shown by the semi-synthetically prepared version to confer opsonic and immunoprotective capability. The synthetic antigens herein are structurally defined molecules wherein the N-acetyl groups are regularly spaced every 6 glucosamine units. A significant portion of the multiple free amine groups in these antigens are likely to be protonated at physiologic pH and will have a major impact on the physical properties, such as tertiary structure and solubility.

The third analog core exemplifying a fully deacetylated PNAG molecule as represented by 61, 62, 63 in FIG. 2B. The third analog core presents a three-dimensional structure or tertiary structure distinguished from naturally derived deacetylated PNG structures and is similar to synthetic PNAGs recently described (Gening et al., Infect. Immun., 78:764, 2010, epub Nov. 30, 2009).

Key building blocks were divided into two batches; in each case, one was converted into an acceptor, the other into a donor. Coupling of the donor and acceptor units provided a key monosaccharide or disaccharide units for use in higher antigen assembly so as to provide an efficient route to large, defined poly-glucosamine structures. To produce the mixed-N-acetyl sequences (Ag 3, 6, and 9), an N-Troc protective group was employed to facilitate selective replacement with an N-acetyl. This chemistry was highly selective and produced a set of three mixed N-acetyl sequences (6, 12, and 18-mer) with one, two and three N-acetyl groups respectively. This corresponds to 16.7% incorporation of N-acetyl, similar to the average degree of N-acetylation found in the most active naturally-derived heterogeneous materials (Maira-Litran et al., Infect. Immun., 73:6752, 2005).

Each protected antigen was reacted with thioacetic acid to install a thioacetate at the reducing end as a conjugation site (See Scheme 2 of Buskas et al., J. Org. Chem., 65:958, 2000). Removal of the protecting groups provided two sets of compounds, the 100% poly-NH₂ sequences (by 61, 62, 63) and the 16.7% N-acetyl substituted structures (thiol oligosaccharides 37, 40, 34). Reaction of a portion of the 100% poly-NH₂ sequences with acetic anhydride under aqueous conditions provided the 100% poly-N-acetyl sequences (58, 59, 60). All synthetic antigens were purified via size exclusion chromatography (BioGel P2 or P4) and fully characterized by ¹H-NMR, ¹³C-NMR and mass spectroscopy.

The PNAG-based library set contains 9 structures comprised of molecules varying by the 3 core unit analogs used and by 3 different molecule lengths (FIG. 2B). Complete analytical characterization (NMR, MS, HPLC, elemental analysis) demonstrated that each molecule was over 98% pure compound.

### Example 2

### Standard Operating Procedure (SOP) 1: Removal of 1° TBS Group

To a solution of the starting tert-butyldimethylsilyl (TBS) containing material (35 mmol) in CH₃CN (500 mL) were added H₂O (50 mL) over 10 minutes. Scandium trifluoromethanesulfonate trihydrate (Sc(OTf)₃, 400 mg, 0.8 mmol) were added and the reaction mixture was stirred at room temperature for 24 hours. The reaction mixture was then diluted with EtOAc (500 mL) and washed with saturated aqueous NaHCO₃ and brine. The organic solution was dried over Na₂SO₄, filtered and concentrated. Purification via silica gel chromatography (EtOAc/Heptanes; 50-100% EtOAc gradient) afforded the desired deprotected product. Typical isolated yields for the product formation varied between 60-92%.

### Example 3

### SOP 2: Removal of Allyl Group

A solution of 1,5-cyclooctadienebis(methyldiphenylphosphine)iridium(I) hexafluorophosphate (Ir cat.; 0.3 mmol) in THF (50 mL) was purged with hydrogen bubbling until a clear yellow solution remained (˜15 minutes). The activated Ir catalyst solution was then purged with nitrogen bubbling for 15 minutes. A solution of the allyl glycoside (10 mmol) in THF (20 mL) was added in one portion to the Ir catalyst solution and the resulting reaction mixture was stirred for 30 minutes. The inert atmosphere was removed and a solution of N-methylmorpholine N-oxide (NMO, 50% aqueous, 20 mL) was added followed by osmium tetroxide (0.03 mmol). The resulting biphasic reaction mixture was stirred in the dark for 2 h, then quenched with 20 mL 1M aqueous Na₂S₂O₄. After vigorous stirring for 1 h, the organic phase was partitioned, diluted with EtOAc (400 mL) and washed with 1M HCl (aq.), H₂O and brine. The organics were dried over Na₂SO₄, filtered and concentrated. Purification via silica gel chromatography (EtOAc/Heptanes; 50-100% EtOAc gradient) afforded the desired hydroxyl product. Typical isolated yields for the product formation varied between 80-95%.

### Example 4

### SOP 3: Tricholoracetimidate Formation

Formation with DBU. A solution of the starting sugar (4 mmol) in CH₂Cl₂ (20 mL) was treated with trichloroacetonitrile (5 mL). To the reaction mixture were added 1,8-diazabicyclo[5.4.0]undec-7-ene (DBU, 0.1 mL, 0.6 mmol) dropwise. The reaction mixture was stirred at room temperature for 1 h, then concentrated to a viscous oil. Purification via filtration through a silica gel plug pre-treated with EtOAc containing 0.1% TEA afforded the desired trichloroacetimidate product. Typical isolated yields for the product formation varied between 80-95%.

Formation with K₂CO₃.

A solution of the starting sugar (4 mmol) in CH₂Cl₂ (20 mL) was treated with trichloroacetonitrile (5 mL). K₂CO₃ (5 g) was added to the reaction mixture and the heterogeneous solution was stirred for 12 h. The reaction mixture was filtered through celite, rinsed with CH₂Cl₂ and concentrated in vacuo. Purification via filtration through a silica gel plug pre-treated with EtOAc containing 0.1% TEA afforded the desired trichloroacetimidate product. Typical isolated yields for the product formation varied between 80-95%.

### Example 5

### SOP 4: Glycoslyation Using Trichloroacetimidate Donors

Glycosyl trichloroacetimidate (12.0 mmol) and glycosyl acceptor (10.0 mmol) were combined, co-evaporated with toluene (3×20 mL) and dried in vacuo for 1 h. The resulting mixture was dissolved in dry CH₂Cl₂ (50 mL) under nitrogen and the reaction mixture was cooled to −20° C. A solution of trimethylsilyl trifluoromethanesulfonate (TMSOTf, 0.10 M in CH₂Cl₂, 0.12 mL, 1.2 mmol) was added dropwise over 10 minutes and the reaction stirred for an additional 30 minutes. The reaction mixture was diluted with CH₂Cl₂ (50 mL) and washed with saturated aqueous NaHCO₃ and brine. The organic solution was dried over Na₂SO₄, filtered and concentrated. Purification via silica gel chromatography (EtOAc/Heptanes; 50-100% EtOAc gradient) afforded the desired coupling product. Typical isolated yields for the product formation varied between 60-97%.

### Example 6

### SOP 5: Removal of N-Troc Group and In Situ N-Acetylation

Starting N-Troc oligosaccharide (0.22 mmol) was dissolved in THF:Ac₂O:AcOH (8:3:1, v:v:v; 20 mL). The reaction mixture was treated with activated Zn (15 mmol) and stirred at room temperature for 1 h. The reaction mixture was diluted with EtOAc, filtered through celite and washed with saturated aqueous NaHCO₃ and brine. Purification via silica gel chromatography (EtOAc/Heptanes; 50-100% EtOAc gradient) afforded the desired N-Acetate product. Typical isolated yields for the product formation varied between 60-90%.

Zinc activation: Zinc (50 g, powdered) was washed with 200 mL each: 2M HCl(aqueous), H₂O, EtOH and THF. The solids were dried in vacuo overnight to a constant weight.

### Example 7

### SOP 6: Thiol Addition to Olefin

Starting allyl glycoside (0.22 mmol) was dissolved in 1,4-dioxane (5 mL). The solution was degassed with nitrogen. Thiol acetic acid (2.2 mmol) and 2,2′-azobis(isobutyronitrile) (0.088 mmol) were added and the reaction mixture was degassed a second time. The reaction was heated to 75° C. for 3 h, then cooled to room temperature and quenched with cyclohexene (0.2 mL). After concentration, the crude reaction mixture was purified via silica gel chromatography (EtOAc/Heptanes; 50-100% EtOAc gradient) to provide the thiolated reaction product. Typical isolated yields for the product formation varied between 70-95%.

### Example 8

### SOP7: Removal of O-Acetate, N-Phthaloyl, and S-Acetate Groups

Starting protected oligosaccharide (0.06 mmol) was dissolved in MeOH (10 mL). Hydrazine hydrate (1 mL) was added and the reaction mixture was heated to 65° C. for 3 h. White precipitates formed upon heating. After 3 h at 65° C., H₂O (5 mL) was added and the reaction mixture was stirred at 65° C. for an additional 12 h. The reaction mixture was concentrated in vacuo and co-evaporated with H₂O (3×5 mL). Purification via size exclusion chromatography (Biogel P-2 Media, 1″×24″ column, gravity pressure, H₂O eluent) afforded the desired fully deprotected oligosaccharides as a mixture of thiol-disulfide products. Typical isolated yields for the product formation varied between 60-85%.

### Example 9

### Synthesis of Monosaccharide Building Blocks

FIG. 3 outlines the reaction scheme for synthesizing the monosaccharide building blocks, which proceeds according to the following steps.

### Synthesis of Building Blocks 6 and 8 for Selective Amine Group Protection of Individual Monosaccharide Units

Glucosamine 1 (90 g, 0.23 mol), (prepared as described in Tetrahedron 1997, 53, 12, 4159) was dissolved in pyridine (405 mL) and triethylamine (36.5 mL). The solution was stirred for 15 minutes followed by the addition of phthalic anhydride (22 g, 0.15 mmol). The reaction mixture was maintained at room temperature in a water bath for 30 minutes. Triethylamine (40.5 mL) and phthalic anhydride (22 g, 0.15 mmol) were added and stirred at room temperature for an additional 45 minutes. The reaction mixture was heated to 90° C. and acetic anhydride (121.5 mL) was added. The reaction mixture was maintained at 90-95° C. for 10 minutes followed by concentration in vacuo to a thick, yellow syrup. The crude product was dissolved in CH₂Cl₂ (1.0 L) and washed with H₂O (3×500 mL). The organic solution was dried over Na₂SO₄, filtered and concentrated to a syrup. The product was recovered via recrystallization from ethanol (300 mL, 190 proof, 0.1% MeOH). Recovered 103 g product 2, 93% yield. To a solution of 2 (103 g, 0.21 mol) in CH₂Cl₂ (700 mL) were added allyl alcohol (62 mL). The reaction mixture was purged with N₂ and cooled to 0° C. SnCl₄ (62 mL) was added dropwise over 1 h. The reaction mixture was kept at 0° C. for 6 h then warmed to room temperature and stirred for 48 h. The solution was poured into ice water (1 L), separated and the organics were washed with 3×500 mL H₂O, dried over Na₂SO₄, filtered and concentrated to a syrup. Recovered 3 (100 g) as a yellow oil. A solution of 3 (1.0 g, 2.1 mmol) was dissolved in MeOH (10 mL) and cooled to 0° C. Acetyl chloride (0.75 mL) was added dropwise over 5 minutes. The solution was allowed to warm to room temperature over 2 h and stirred for an additional 48 h. The reaction mixture was concentrated in vacuo to afford 4 (0.7 g) as a white solid. Starting monomer 4 (49.2 g, 0.141 mol) was dissolved in pyridine (160 mL) and cooled to 0° C. under N₂. A solution of tert-butyldimethylsilyl chloride (TBSCl, 21.3 g, 0.141 mol) in CH₂Cl₂ (70 mL) was added over 1 h and the temperature was maintained at <1° C. The reaction mixture was stirred for an additional 1 h at 0° C., then a second portion of TBSCl (2.0 g, 0.014 mol) in CH₂Cl₂ (7 mL) was added and stirred at 0° C. for 1 h. The reaction mixture was warmed to room temperature for 2 h and then re-cooled to 0° C. Acetic anhydride (80 mL) was added over 1 h at 0° C. and the solution was warmed to room temperature overnight. After 12 h at rt, the reaction mixture was poured onto ice water (1 L) and stirred for 1 h. The biphasic solution was extracted with EtOAc in 3 portions (1 L, then 2×250 mL). The combined organics were concentrated and the product recrystallized from hot ethanol (300 mL) to give 5 (66 g, 86% yield) as a white solid. TBS removal was performed as described in SOP 1 using 5 (32 g, 59 mmol) and Sc(OTf)₃ (400 mg, 0.8 mmol). Product 6 was formed in 88% yield (22.5 g). Allyl removal was performed as described in SOP 2 using Ir catalyst (1.0 g, 1.2 mmol), 5 (70 g, 127 mmol), 50% aqueous NMO (100 mL) and OSO₄ (20 mg, 0.08 mmol). Product 7 was formed in 94% yield (61 g). Glycosyl trichloroacetimidate 8 was formed as described in SOP 3a using 7 (61 g, 120 mmol), trichloroacetonitrile (30 mL) and DBU (1 mL). Product 8 was formed in 96% yield (75.9 g).

### Synthesis of Monosaccharide Building Blocks 14 and 47 for Selective Protection and Subsequent Acetylation of Individual Monosaccharide Units

Starting sugar 1 (50 g, 130 mmol; see FIG. 3) was dissolved in THF (350 mL). With constant stirring, a solution of NaHCO₃ (22 g in 170 mL H₂O) was added slowly over 20 minutes. The reaction mixture was stirred for an additional 20 minutes. Troc-Cl (18.5 mL, 60 mmol) was added over 10 minutes followed by a second portion (18.5 mL, 60 mmol). After 1 hour, the reaction mixture was diluted with H₂O (500 mL) and EtOAc (1 L). The organics were partitioned and washed with 2×250 mL brine, dried over Na₂SO₄, filtered and concentrated. Product 9 was recovered in quantitative yield (69 g). To a solution of 9 (78 g, 0.15 mol) in CH₂Cl₂ (350 mL) were added allyl alcohol (43 mL). The reaction mixture was purged with N₂ and cooled to 0° C. SnCl₄ (43 mL) was added dropwise over 1 h. The reaction mixture was kept at 0° C. for 6 h then warmed to room temperature and stirred for 24 h. The solution was poured into ice water (1 L), separated and the organics were washed with 3×500 mL H₂O, dried over Na₂SO₄, filtered and concentrated to a syrup. Recovered 10 (72 g) as a yellow oil in 92% yield. A solution of 10 (72 g, 138 mmol) was dissolved in MeOH (300 mL) and cooled to 0° C. Acetyl chloride (30 mL) was added dropwise over 5 minutes. The solution was allowed to warm to room temperature over 2 h and stirred for an additional 48 h. The reaction mixture was concentrated in vacuo to afford 11 (54 g) as a yellow syrup that was used in the next step without further purification. Starting monomer 11 (54 g, 0.138 mol) was dissolved in pyridine (180 mL) and cooled to 0° C. under N₂. A solution of tert-butyldimethylsilyl chloride (TBSCl, 23.7 g, 0.157 mol) in CH₂Cl₂ (75 mL) was added over 1 h and the temperature was maintained at <1° C. The reaction mixture was stirred for an additional 1 h at 0° C., then a second portion of TBSCl (4.0 g, 0.028 mol) in CH₂Cl₂ (10 mL) was added and stirred at 0° C. for 1 h. The reaction mixture was warmed to room temperature for 2 h and then re-cooled to 0° C. Acetic anhydride (80 mL) was added over 1 h at 0° C. and the solution was warmed to room temperature overnight. After 12 h at rt, the reaction mixture was poured onto ice water (1 L) and stirred for 1 h. The biphasic solution was extracted with EtOAc in 3 portions (1 L, then 2×250 mL). The combined organics were concentrated and the product recrystallized from hot ethanol (300 mL) to give 12 (65 g, 80% yield) as a white solid. Allyl removal was performed as described in SOP 2 using Ir catalyst (0.6 g, 0.7 mmol), 12 (32 g, 54 mmol), 50% aqueous NMO (50 mL) and OSO₄ (10 mg, 0.04 mmol). Product 13 was formed in 70% yield (21 g, 38 mmol). Glycosyl trichloroacetimidate 14 was formed as described in SOP 3 using 13 (21 g, 38 mmol), trichloroacetonitrile (30 mL) and K₂CO₃ (20 g). Product 14 was formed in 97% yield (25.5 g).

### Example 10

### Synthesis of Disaccharide Building Blocks

FIG. 4A-4D outline reaction schemes for synthesizing various disaccharide building blocks, including those depicted in FIG. 4A which proceeds according to the following steps. In FIG. 4A, coupling was performed as described in SOP 4 using trichloroacetimidate 14 (13.2 g, 19.1 mmol), acceptor 6 (6.34 g, 14.7 mmol) and TMSOTf (2.94 mL, 0.5 M in CH₂Cl₂, 1.47 mmol). Product 15 was formed in 80% yield (11.04 g). Allyl removal was performed as described in SOP 2 using Ir catalyst (0.25 g, 0.3 mmol), 15 (10 g, 10 mmol), 50% aqueous NMO (25 mL) and 0504 (5 mg, 0.02 mmol). Product 16 was formed in 90% yield (9 g, 9 mmol). Glycosyl trichloroacetimidate 17 was formed as described in SOP 3 using 16 (9 g, 9 mmol), trichloroacetonitrile (20 mL) and K₂CO₃ (10 g). Product 17 was formed in quantitative yield (10 g).

The reaction schemes depicted in FIGS. 4B-4D employ monosaccharide building block starting materials (as shown), but essentially the same steps and SOPs (-1, -2, -3, and -4) as described above. For example, in FIG. 4C, coupling was performed as described in SOP 4 using: trichloroacetimidate 14 (12 g, 17.2 mmol), acceptor 47 (6.86 g, 14.3 mmol) and TMSOTf (1.43 mL, 1.0 M in CH₂Cl₂, 1.43 mmol). Product 48 was formed in 98% yield (14.2 g, 14 mmol).

### Example 11

### Synthesis of mixed-N-acetyl oligo-β-(1→6)-glucosamine Structures

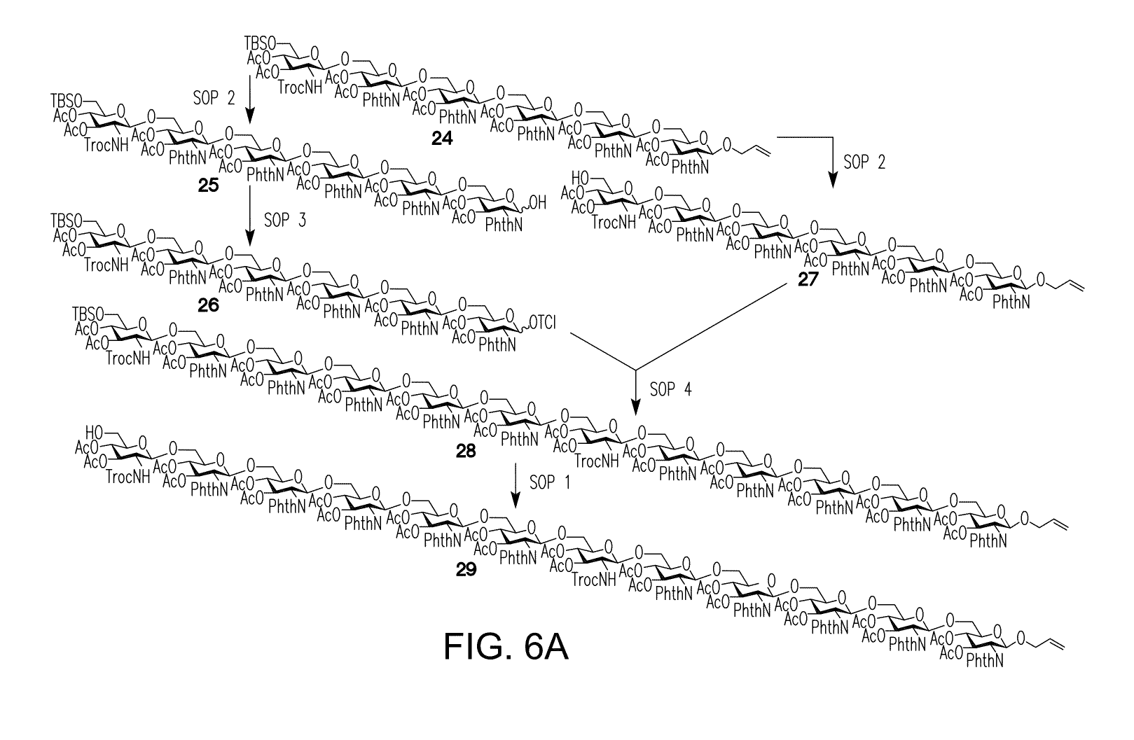

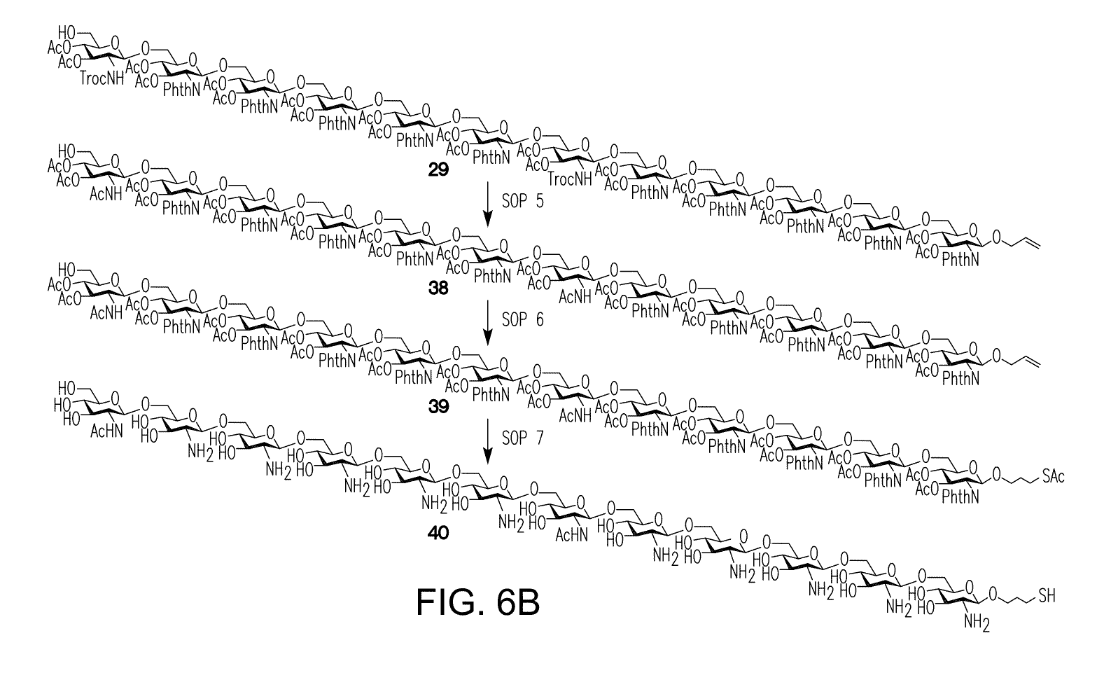

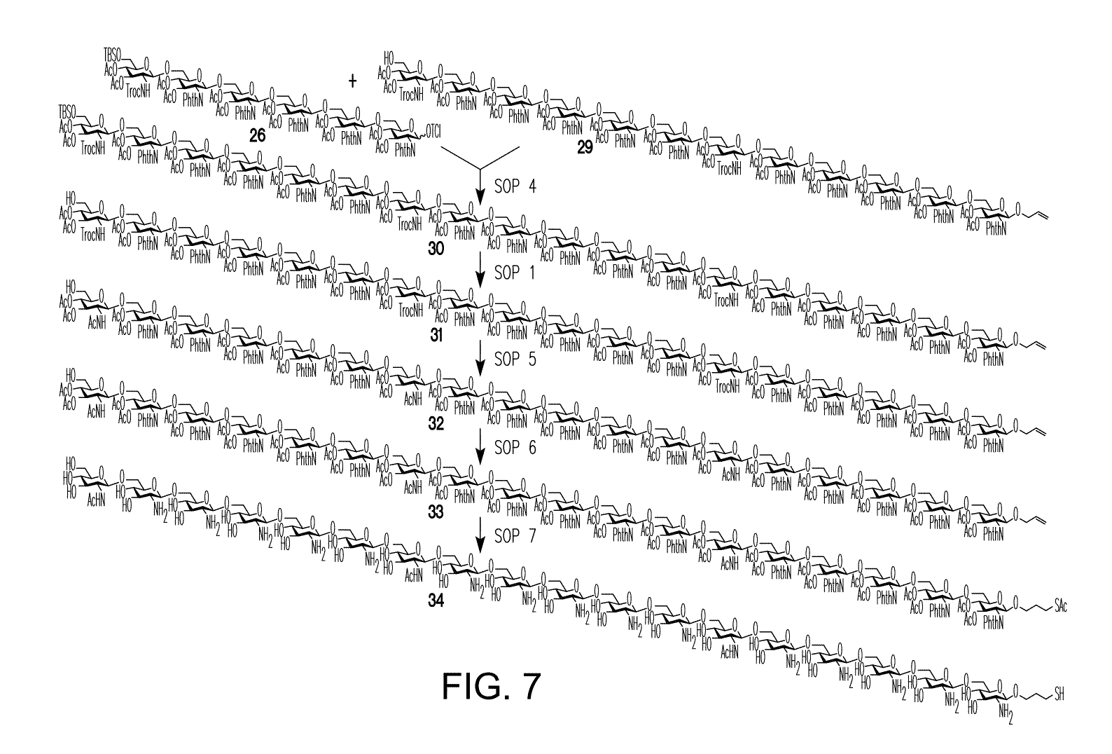

FIG. 5A-5B depict a reaction scheme for synthesizing a mixed-N-acetyl oligo-β-(1→6)-glucosamine 6-mer (37 in FIG. 2B), which proceeds according to the following steps. Utilization of monosaccharide and disaccharide building blocks described in FIGS. 3 and 4A-4D in conjunction with the indicated SOPs additionally described in Examples 2-8 exemplify the methodologies and reagents for synthesizing mixed-N-acetyl oligo-β-(1→6)-glucosamine structures, including oligosaccharides 37, 40, and 34 in FIG. 2B, the syntheses of which are outlined in FIGS. 5A, 5B (6-mer, 37), FIGS. 6A, 6B (12-mer, 40), and FIGS. 7A, 7B (18-mer, 34) and further described below.

### Synthesis of mixed-N-acetyl oligo-β-(1→6)-glucosamine 6-mer thiol 37

Referring to FIG. 5A, coupling was performed as described in SOP 4 using trichloroacetimidate 8 (65 g, 101 mmol), acceptor 6 (36.3 g, 84 mmol) and TMSOTf (8.4 mL, 1.0 M in CH₂Cl₂, 8.4 mmol). Product 18 was formed in 96% yield (75 g). TBS removal was performed as described in SOP 1 using 18 (35 g, 38 mmol) and Sc(OTf)₃ (400 mg, 0.8 mmol). Product 21 was formed in 84% yield (26 g, 32 mmol). Allyl removal was performed as described in SOP 2 using Ir catalyst (0.50 g, 0.6 mmol), 18 (50 g, 55 mmol), 50% aqueous NMO (50 mL) and OSO₄ (10 mg, 0.04 mmol). Product 19 was formed in 77% yield (37.5 g, 42.5 mmol). Glycosyl trichloroacetimidate 20 was formed as described in SOP 3 using 19 (37 g, 42 mmol), trichloroacetonitrile (20 mL) and DBU (0.6 mL). Product 20 was formed in quantitative yield (44 g). Coupling was performed as described in SOP 4 using trichloroacetimidate 20 (35 g, 34 mmol), acceptor 21 (23 g, 28 mmol) and TMSOTf (2.8 mL, 1.0 M in CH₂Cl₂, 2.8 mmol). Product 22 was formed in 82% yield (38 g). TBS removal was performed as described in SOP 1 using 22 (37 g, 22 mmol) and Sc(OTf)₃ (400 mg, 0.8 mmol). Product 23 was formed in 68% yield (24 g, 15 mmol).

Referring now to FIG. 5B, coupling was performed as described in SOP 4 using trichloroacetimidate 17 (9.9 g, 9.2 mmol), acceptor 23 (12 g, 7.7 mmol) and TMSOTf (0.77 mL, 1.0 M in CH₂Cl₂, 0.77 mmol). Product 24 was formed in 83% yield (15.7 g, 6.4 mmol). TBS removal was performed as described in SOP 1 using 24 (4.55 g, 1.8 mmol) and Sc(OTf)₃ (60 mg, 0.12 mmol). Product 27 was formed in 83% yield (3.6 g, 1.5 mmol). Exchange of N-Troc for N-Acetate groups was performed as described in SOP 5 using 27 (0.50 g, 0.23 mmol) and Zn (1 g) in THF:Ac₂O:AcOH (20 mL). Product 35 was formed in 51% yield (0.25 g, 0.12 mmol). Thiol addition was performed as described in SOP 6 using 35 (0.20 g, 0.09 mmol), HSAc (0.5 mL) and AlBN (50 mg). Product 36 was formed in 78% yield (0.16 g, 0.07 mmol). Deprotection was performed as described in SOP 7 using 36 (0.15 g, 0.06 mmol) and hydrazine (1 mL). Product 37 was formed in 90% yield (0.07 g, 0.054 mmol).

### Synthesis of mixed-N-acetyl oligo-β-(1→6)-glucosamine 12-mer thiol 40

As depicted in FIGS. 6A and 6B, synthesis of the mixed-N-acetyl oligo-β-(1→6)-glucosamine 12-mer thiol proceeded according to the following steps. With reference to FIG. 6A, allyl removal in 24 was performed as described in SOP 2 using Ir catalyst (0.25 g, 0.3 mmol), 24 (7.5 g, 3.0 mmol), 50% aqueous NMO (25 mL) and 0504 (5 mg, 0.04 mmol). Product 25 was formed in 93% yield (7.0 g, 2.8 mmol). Glycosyl trichloroacetimidate 26 was formed from 25 as described in SOP 3b using 25 (7.0 g, 2.8 mmol), trichloroacetonitrile (10 mL) and K₂CO₃ (7 g). Product 26 was formed in 86% yield (6.3 g, 2.4 mmol). TBS removal of was performed as described in SOP 1 using 24 (4.55 g, 1.8 mmol) and Sc(OTf)₃ (60 mg, 0.12 mmol). Product 27 was formed in 83% yield (3.6 g, 1.5 mmol). Coupling of 26 and 27 was performed as described in SOP 4 using trichloroacetimidate 26 (4.2 g, 1.6 mmol), acceptor 27 (3.2 g, 1.36 mmol) and TMSOTf (1.36 mL, 0.1 M in CH₂Cl₂, 0.136 mmol). Product 28 was formed in 65% yield (4.2 g, 0.88 mmol). TBS removal in 28 was performed as described in SOP 1 using 28 (4.0 g, 0.84 mmol) and Sc(OTf)₃ (60 mg, 0.12 mmol). Product 29 was formed in 63% yield (2.6 g, 0.55 mmol);

Referring now to FIG. 6B, (6) exchange of N-Troc for N-Acetate groups in 29 was performed as described in SOP 5 using 29 (0.47 g, 0.10 mmol) and Zn (1 g) in THF:Ac₂O:AcOH (20 mL). Product 38 was formed in 90% yield (0.4 g, 0.09 mmol). Thiol addition to 38 was performed as described in SOP 6 using 38 (0.40 g, 0.09 mmol), HSAc (0.5 mL) and AlBN (50 mg). Product 39 was formed in 57% yield (0.23 g, 0.51 mmol). Deprotection of 39 and formation of 40 was performed as described in SOP 7 using 39 (0.22 g, 0.065 mmol) and hydrazine (3 mL). Product 40 was formed in 73% yield (0.10 g, 0.047 mmol).

### Synthesis of mixed-N-acetyl oligo-β-(1→6)-glucosamine 18-mer thiol 34

As depicted in FIG. 7, synthesis of the mixed-N-acetyl oligo-β-(1→6)-glucosamine 18-mer thiol proceeded according to the following steps. Coupling of 26 and 29 was performed as described in SOP 4 using trichloroacetimidate 26 (1.17 g, 0.455 mmol), acceptor 29 (1.41 g, 0.303 mmol) and TMSOTf (0.3 mL, 0.1 M in CH₂Cl₂, 0.03 mmol). Product 30 was formed in 67% yield (1.4 g, 0.20 mmol). TBS removal in 30 was performed as described in SOP 1 using 30 (2 g, 0.29 mmol) and Sc(OTf)₃ (40 mg, 0.08 mmol). Product 31 was formed in 90% yield (0.18 g, 0.26 mmol). Exchange of N-Troc for N-Acetate groups in 31 was performed as described in SOP 5 using 31 (0.73 g, 0.10 mmol) and Zn (1 g) in THF:Ac₂O:AcOH (20 mL). Product 32 was formed in 92% yield (0.6 g, 0.092 mmol). Thiol addition to 32 was performed as described in SOP 6 using 32 (0.6 g, 0.092 mmol), HSAc (0.₅ mL) and AlBN (50 mg). Product 33 was formed in 83% yield (0.5 g, 0.076 mmol). Deprotection of 33 and formation of 34 was performed as described in SOP 7 using 33 (0.5 g, 0.076 mmol) and hydrazine (3 mL). Product 34 was formed in 79% yield (0.19 g, 0.06 mmol).

Table 1 provides supporting characterization data for selected antigens and intermediates described in Example 11.

TABLE 1

| | | | NMR | | Mass Spec | | |
|---|---|---|---|---|---|---|---|
| Series | Description | Molecule # | ¹H | ¹³C | Theo. | ESI (Pos. Mode) | MALDI |
| 6-mer | Troc/Phth | 27 | ✓ | | 2355 | 2376 (M + Na⁺) | |
| | NHAc/Phth | 35 | ✓ | | 2221 | 2277 (M⁺ + HOAc) | |
| | NHAc/Phth/SAc | 36 | ✓ | | 2340 | 2353 (M⁺) | |
| | NHAc/NH₂ | 37 | ✓ | ✓ | 1101 | 1101 (M⁺) | |
| 12-mer | Troc/Phth | 29 | ✓ | | 4652 | | 4674 (M + Na⁺) |
| | NHAc/Phth | 38 | ✓ | | 4386 | | 4409 (M + Na⁺) |
| | NHAc/Phth/SAc | 39 | ✓ | | 4459 | 4482 (M + Na⁺) | |
| | NHAc/NH₂ | 40 | ✓ | ✓ | 2109 | | 2110 (M + H⁺) |
| 18-mer | Troc/Phth | 31 | ✓ | | 6949 | | 6973 (M + Na⁺) |
| | NHAc/Phth | 32 | ✓ | | 6549 | | 6572 (M + Na⁺) |
| | NHAc/Phth/SAc | 33 | ✓ | | 6622 | | 6668 (M⁺ + HOAc) |
| | NHAc/NH₂ | 34 | ✓ | ✓ | 3117 | 1039 (M⁺³) | 3140 (M + Na⁺) |

In Table 1, protein assays were performed according to the method of Bradford, M. Anal. Biochem. 1976, 72, 248. Maldi analysis was performed using 2,5-dihydroxybenzoid acid as a matrix. Copy numbers were determined by the formula: copy number=[Maldi_{(observed)}−76,000_{(Maldi of BSA alone)}]/antigen MW. Carbohydrate content in KLH sample was extrapolated from BSA using the formula: KLH carbohydrate content=BSA carbohydrate content/2.65.

### Example 12

### Conjugation of mixed-N-acetyl oligo-β-(1→6)-glucosamine thiols to BSA and KLH

### Conjugation of mixed-N-acetyl oligo-β-(1→6)-glucosamine 6-mer thiol 34 to BSA and KLH

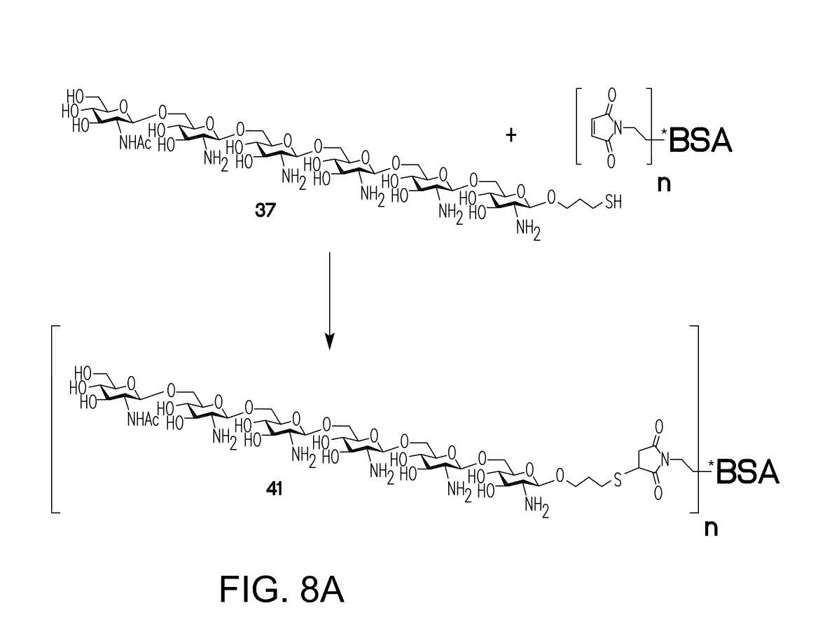

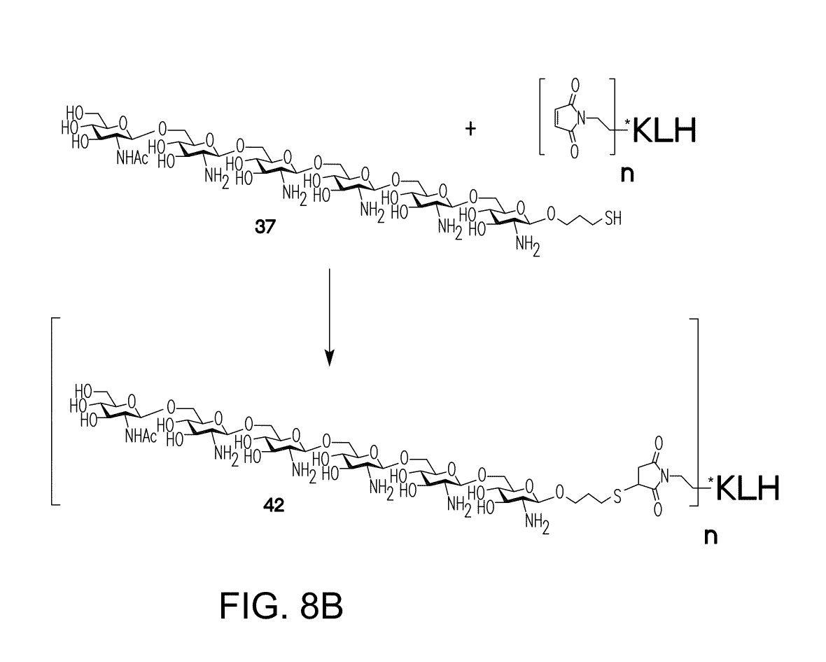

FIGS. 8A and 8B depict reaction schemes for conjugating a mixed-N-acetyl oligo-β-(1→6)-glucosamine 6-mer thiol 37 to BSA and KLH as follows. First, a conjugation stock solution of hexamer thiol 37 was prepared by dissolving the hexamer thiol 37 (4.2 mg, 3.81 μmol) in water (0.300 mL). A solution of tris(2-carboxyethyl)phosphine (TCEP) in water (40 μL, 0.05 M, 1.95 μmol) was added and stirred for 1 hour. Imject® Conjugation Buffer (Pierce, 300 μL) was added to provide a stock solution for conjugation to BSA (FIG. 8A) and KLH (FIG. 8B).

With reference to FIG. 8A, the conjugation stock solution of hexamer thiol 37 (500 μL, 3.0 μmol) was added to a solution of maleimide-activated bovine serum albumin (Imject® BSA, Pierce, Rockford, Ill.) (5 mg, ˜1.5 μmol maleimide) in Imject® Conjugation Buffer (Pierce, 250 μL diluted with 250 μL water) and the resulting solution stirred for 18 hours at room temperature. The reaction mixture was purified by de-salting on D-Salt P-6000 10 mL column (Pierce, Rockford, Ill.). The column was pre-equilibrated with 30 mL of purification buffer (Pierce, Prod. No. 77159), the crude material loaded onto the column and eluted with purification buffer. 1-mL fractions were collected and analyzed for protein content by absorbance at 280 nm (A₂₈₀). Fractions containing protein were combined and lyophilized to give the desired hexamer-BSA conjugate 41.

Turning now to FIG. 8B, the conjugation stock solution of hexamer thiol 37 (140 μL, 0.86 μmol) was added to a solution of maleimide-activated keyhole limpet hemocyanin (Imject® KLH, Pierce, Rockford, Ill.) (5 mg, ˜0.43 μmol maleimide) in water (0.5 mL) was added and the resulting solution stirred overnight at room temperature. The reaction mixture was purified by de-salting on D-Salt P-6000 10 mL column (Pierce, Rockford, Ill.). The column was pre-equilibrated with 30 mL of purification buffer (Pierce, Prod. No. 77159), the crude material loaded onto the column and eluted with purification buffer. 1-mL fractions were collected and analyzed for protein content by absorbance at 280 nm (A₂₈₀). Fractions containing protein were combined and lyophilized to give the desired hexamer-KLH conjugate 42.

### Conjugation of mixed-N-acetyl oligo-β-(1→6)-glucosamine 12-mer thiol 40 to BSA and KLH

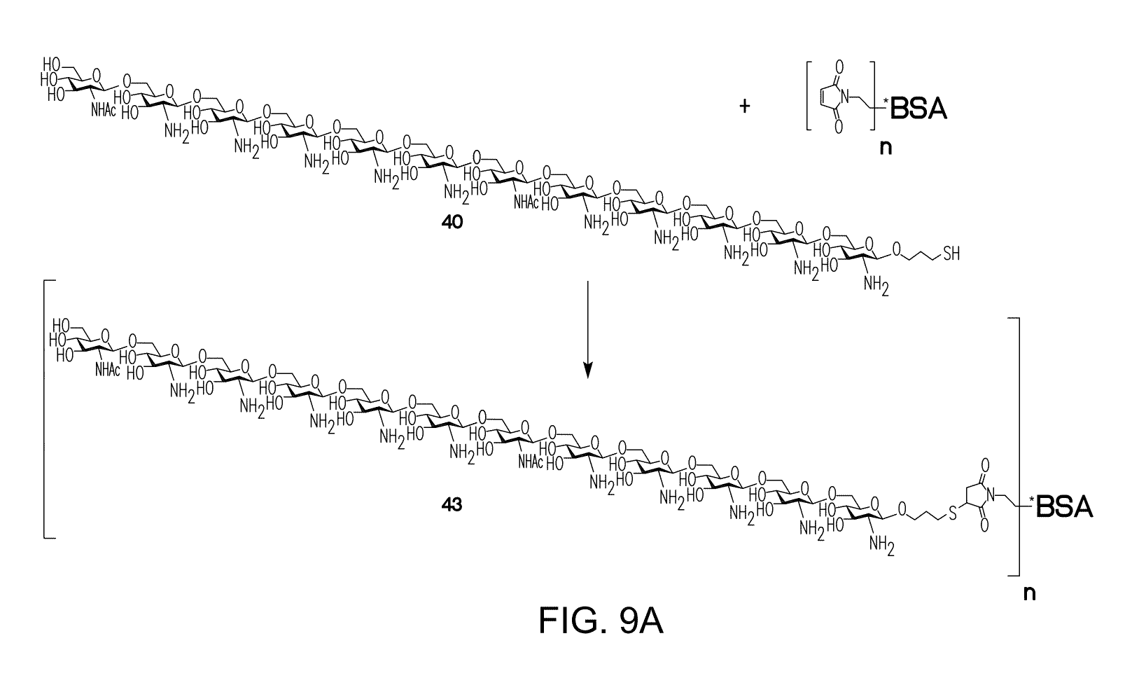

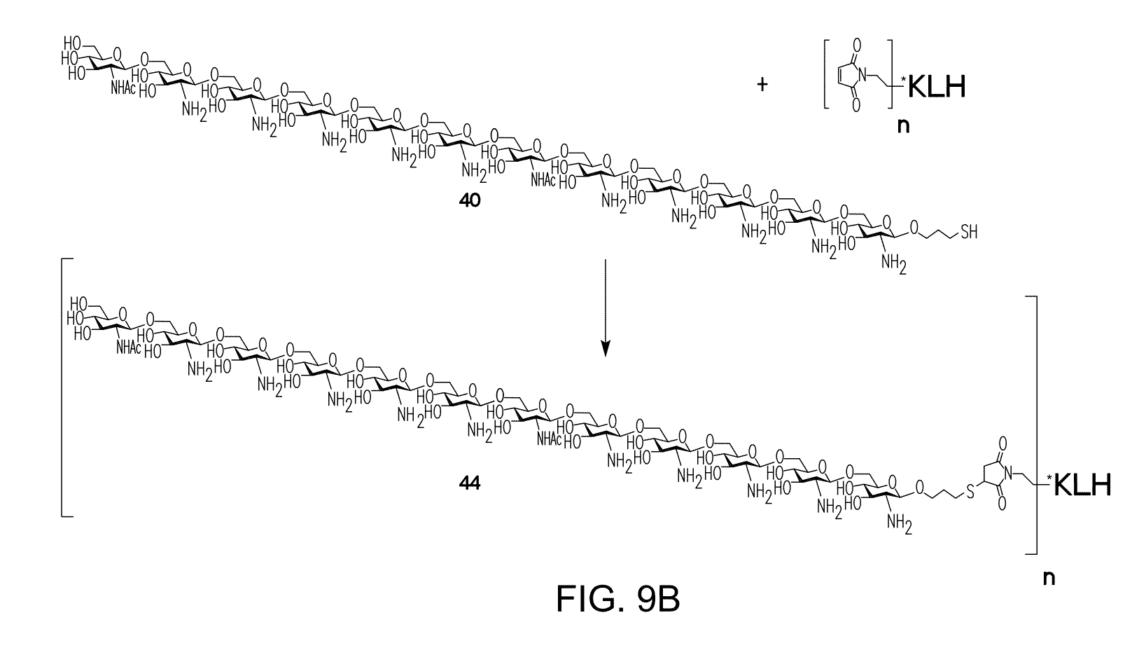

FIGS. 9A and 9B depict reaction schemes for conjugating mixed-N-acetyl oligo-β-(1→6)-glucosamine 12-mers (Ag 9 in FIG. 2B) to BSA and KLH as follows. First, a conjugation stock solution of 12-mer thiol 40 was prepared by dissolving the 12-mer 40 (8.1 mg, 3.84 μmol) in water (300 μL). A solution of tris(2-carboxyethyl)phosphine (TCEP) in water (30 μL, 0.05 M, 1.5 μmol) was added and stirred for 1 hour. Imject® Conjugation Buffer (Pierce, 300 μL) was added to provide a stock solution for conjugation to BSA (FIG. 9A) and KLH (FIG. 9B).

With reference to FIG. 9A, the conjugation stock solution of 12-mer thiol 40 (500 μL, 3.0 μmol) was added to a solution of maleimide-activated bovine serum albumin (Imject® BSA, Pierce, Rockford, Ill.) (5 mg, ˜1.5 μmol maleimide) in Imject® Conjugation Buffer (Pierce, 250 μL diluted with water, 250 μL) and the resulting solution stirred for 18 hours at room temperature. The reaction mixture was purified by de-salting on D-Salt P-6000 10 mL column (Pierce, Rockford, Ill.). The column was pre-equilibrated with 30 mL of purification buffer (Pierce, Prod. No. 77159), the crude material loaded onto the column and eluted with purification buffer. 1-mL fractions were collected and analyzed for protein content by absorbance at 280 nm (A₂₈₀). Fractions containing protein were combined and lyophilized to give the desired 12-mer-BSA conjugate 43.

Turning now to FIG. 9B, the conjugation stock solution of 12-mer thiol 40 (140 μL, 0.86 μmol) was added to a solution of maleimide-activated keyhole limpet hemocyanin (Imject® KLH, Pierce, Rockford, Ill.) (5 mg, ˜0.43 μmol maleimide) in water (0.5 mL) was added and the resulting solution stirred overnight at room temperature. The reaction mixture was purified by de-salting on D-Salt P-6000 10 mL column (Pierce, Rockford, Ill.). The column was pre-equilibrated with 30 mL of purification buffer (Pierce, Prod. No. 77159), the crude material loaded onto the column and eluted with purification buffer. 1-mL fractions were collected and analyzed for protein content by absorbance at 280 nm (A₂₈₀). Fractions containing protein were combined and lyophilized to give the desired 12-mer-KLH conjugate 44.

### Conjugation of mixed-N-acetyl oligo-β-(1→6)-glucosamine 18-mer thiol 34 to BSA and KLH

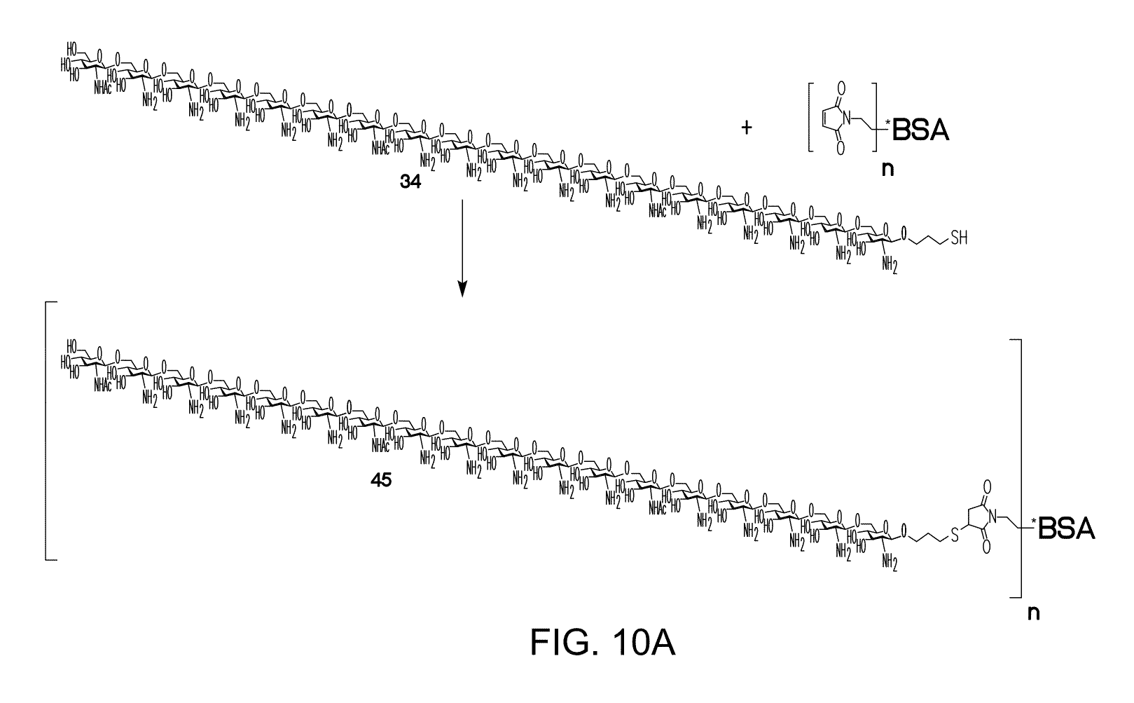

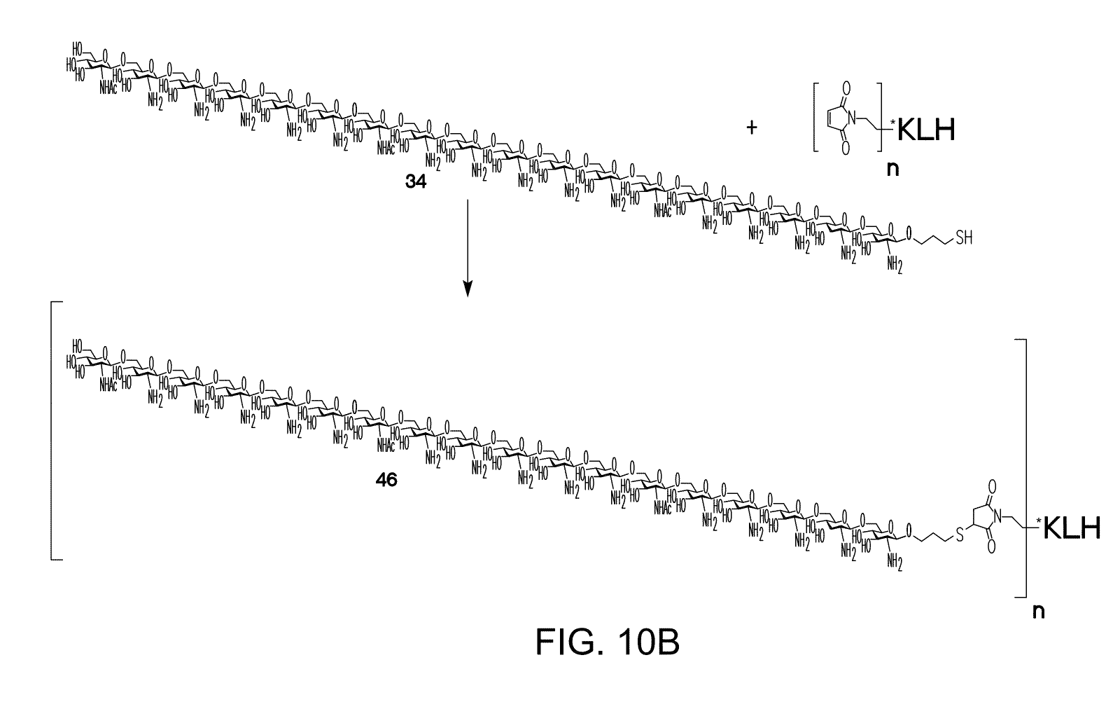

FIGS. 10A and 10B depict reaction schemes for conjugating mixed-N-acetyl oligo-β-(1→6)-glucosamine 18-mers (Ag 3 in FIG. 2B) to BSA and KLH as follows. First, a conjugation stock solution of 18-mer thiol 34 was prepared by dissolving the 18-mer 34 (12 mg, 3.8 μmol) in water (0.3 mL). A suspension of tris(2-carboxyethyl)phosphine (TCEP)-bound agarose resin in water (200 4, ˜1 μmol) was added and stirred for 1 hour. The TCEP-resin was filtered and to the filtrate was added Imject® Conjugation Buffer (Pierce, 300 μL) to provide a stock solution for conjugation to KLH (FIG. 10A) and BSA (FIG. 10B).

With reference to FIG. 10A, the conjugation stock solution of 18-mer thiol 34 (0.5 mL, 3.0 μmol) was added to a solution of maleimide-activated bovine serum albumin (Imject® BSA, Pierce, Rockford, Ill.) (5 mg, ˜1.5 μmol maleimide) in Imject® Conjugation Buffer (Pierce, 250 μL diluted with water, 2500 μL) and the resulting solution stirred for 18 hours at room temperature. The reaction mixture was purified by de-salting on D-Salt P-6000 10 mL column (Pierce, Rockford, Ill.). The column was pre-equilibrated with 30 mL of purification buffer (Pierce, Prod. No. 77159), the crude material loaded onto the column and eluted with purification buffer. 1-mL fractions were collected and analyzed for protein content by absorbance at 280 nm (A₂₈₀). Fractions containing protein were combined and lyophilized to give the desired 18-mer-BSA conjugate 45.

Turning now to FIG. 10B, the conjugation stock solution of 18-mer thiol 34 (0.14 mL, 0.86 μmol) was added to a solution of keyhole limpet hemocyanin (Imject® KLH, Pierce, Rockford, Ill.) (5 mg, ˜0.43 μmol maleimide) in water (500 μL) and the resulting solution stirred for 18 hours at room temperature. The reaction mixture was purified by de-salting on D-Salt P-6000 10 mL column (Pierce, Rockford, Ill.). The column was pre-equilibrated with 30 mL of purification buffer (Pierce, Prod. No. 77159), the crude material loaded onto the column and eluted with purification buffer. 1-mL fractions were collected and analyzed for protein content by absorbance at 280 nm (A₂₈₀). Fractions containing protein were combined and lyophilized to give the desired 18-mer-KLH conjugate 46.

Table 2 provides supporting characterization data for the antigen conjugates described in Example 12.

TABLE 2

| | | Protein Assay | | | | % Carbohydrate | | |
|---|---|---|---|---|---|---|---|---|
| | | BSA | KLH | | Antigen | Ave Copy | | |
| Conjugate # | Description | (mg/mL) | (mg/mL) | MALDI | MW | Number | MALDI | Sample |
| 41 | S-6Mix-BSA | 1.62 | | 100,900 | 1101 | 23 | 23% | |
| 42 | S-6Mix-KLH | | 0.85 | | 1101 | | | 9% |
| 43 | S-12Mix-BSA | 0.98 | | 112,100 | 2109 | 17 | 30% | |
| 44 | S-12MiX-KLH | | 1.32 | | 2109 | | | 11% |
| 45 | S-18Mix-BSA | 1.33 | | 126,251 | 3117 | 16 | 38% | |
| 46 | S-18Mix-KLH | | 1.21 | | 3117 | | | 14% |

In Table 2, protein assays, Maldi analysis, copy numbers, and carbohydrate content were determined as described above with reference to Table 1.

### Example 13

### Serum Antibody Production and Purification

Antisera to antigen-KLH conjugates were raised in New Zealand white rabbits by four subcutaneous injections of antigen-KLH conjugate over 13 weeks. A pre-immune bleed generated 5 mL of baseline serum from each rabbit. The prime injection (10 μg antigen equivalent) was given as an emulsion in complete Freund's adjuvant (CFA). Subsequent injections (5 μg antigen equivalent) were given at three week intervals in incomplete Freund's adjuvant (IFA). Rabbits were bled every two weeks commencing one week after the third immunization. Approximately 25-30 mL of serum per rabbit was generated for each bleeding event, and was aliquoted into 1-mL aliquots and frozen at −80° C. Serum was analyzed by ELISA against the corresponding antigen-BSA conjugate as described in Example 3 below.

Affinity purification of antisera was conducted with serum from the third bleed from each rabbit. Affinity purification was carried out by coupling of antigen-BSA conjugates to CNBr-activated Sepharose 4B. Briefly, CNBr-activated Sepharose 4B (0.8 g, 2.5 ml of final gel volume) was washed and re-swelled on a sintered glass filter with 1 mM HCl, then coupling buffer (0.1M NaHCO₃, 0.25M NaCl, pH 8.5). Antigen-BSA conjugate (1 mg) was dissolved in coupling buffer, mixed with the gel suspension and incubated overnight at 40° C. Unreacted active groups were capped with glycine buffer (0.2M, pH 8.1) and excess adsorbed conjugated was washed away with coupling buffer, then acetate buffer (0.1 M containing 0.5M NaCl, pH 4.3). The column was equilibrated with phosphate buffered saline (PBS), pH 7.7.

Antisera were affinity purified by diluting clear antiserum (5 mL) 1:1 with PBS pH 7.7 and applying the diluted antisera to the affinity column at the rate of 0.3 ml/min and absorbance of eluate was monitored at 280 nm. Unbound material (flow through) was collected and analyzed by ELISA using the general ELISA procedure. The column was washed with PBS until A280 reached baseline. Bound antibodies were eluted with 0.2M glycine (pH 1.85) into one fraction until the A280 returned to baseline. Fractions were neutralized with 1M Tris-HCl, pH 8.5 immediately after collection and the OD at 280 nm was determined. ELISA analysis was conducted using the corresponding antigen-BSA conjugate according to the general ELISA protocol to confirm the recovered antibody and the removal of all the antibodies from the original serum. Antibody quantification was determined by A280 reading of the antibody (a small amount was diluted to give an OD value of about 1.0) and this value was divided by the extinction coefficient of IgG, 1.4, to give mg/mL. The solutions were concentrated to ˜1-2 mg/mL, dialyzed against PBS with 0.02% sodium azide, aliquoted and frozen at −80° C.

### Example 14

### ELISA Plate Preparation and Use

An oligosaccharide-BSA conjugate solution was prepared by dissolving the conjugate in carbonate buffer (1.59 g Na₂CO₃, 2.93 g NaHCO₃, 0.20 g NaN₃, dissolved and diluted to 1 L in H₂O, final pH 9.5) at a concentration of 5-10 μg/mL. COSTAR flat bottom EIA 8-well strips were incubated with oligosaccharide-BSA conjugate solution (100 μL per well) for 24 hours in a humidity chamber to coat the well surfaces. Coating solution was removed, each well was rinsed twice with water, and dried on a paper towel. Blocking solution (0.1% BSA in PBS with 0.02% thimerosal, 200 μL) was added to each well and incubated for 2 hours in a humidity chamber. Blocking solution was removed; each well was rinsed with water and dried on a paper towel.

Serum samples were prepared by 1:5 serial dilutions starting from a 1:1,000 dilution of serum in 0.1% BSA in PBS with 0.02% thimerosal. Diluted serum (100 μL) was added to each well and incubated for 2 hours at room temperature in a humidity chamber. The serum solution was removed, the wells were rinsed twice with water and dried on a paper towel. Goat anti-rabbit-HRP conjugate solution (100 μL/well) was added and incubated for 2 hours at room temperature in a humidity chamber. The HRP-conjugate solution was removed, and wells were washed three times with PBS/0.02% thimerosal/0.05% tween-20, twice with water, and dried on a paper towel. TMB solution (100 μL/well) was added and developed at room temperature. The reaction was stopped by the addition of 1N HCl (100 μL/well) and the wells were read immediately at A450 (absorbance at 450 nm). The titer of the test serum was designated as the dilution which gave an optical density (OD₄₅₀) reading of 0.1 above background.

### Example 15

### Rabbit Immunogenicity and ELISA Results

Immunogenicity of Synthetic Antigen-KLH Conjugates in Rabbits (ELISA).

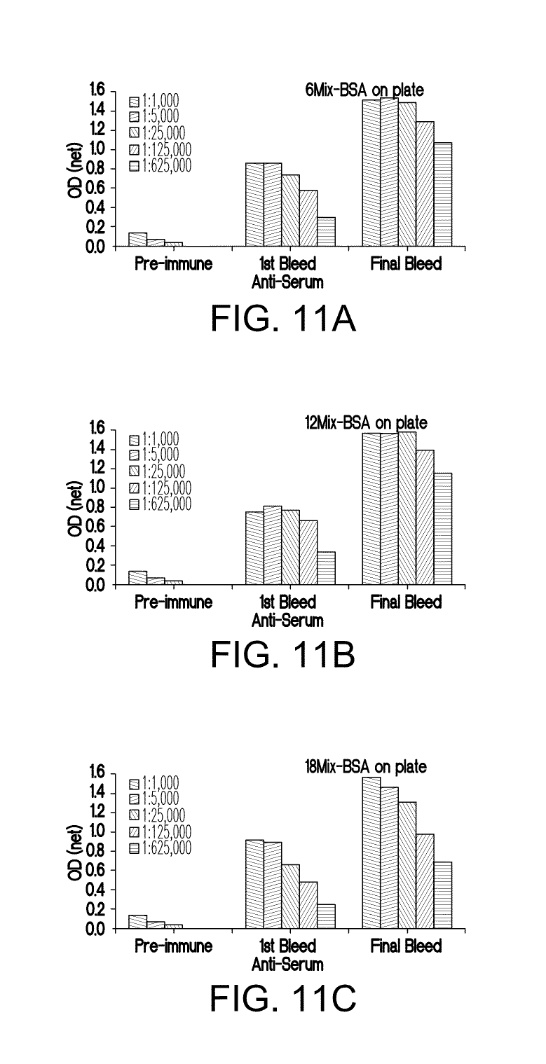

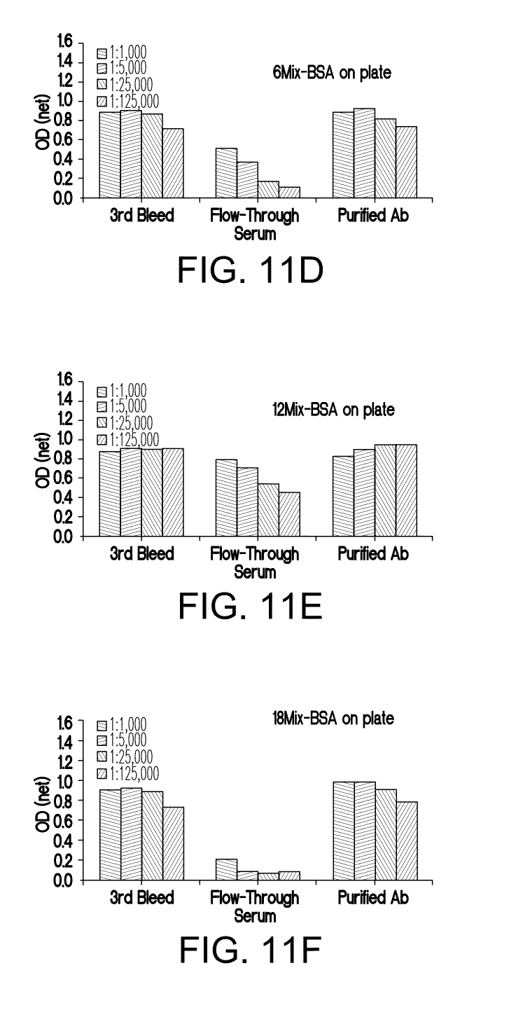

FIGS. 11A-11C depict IgG antibody titers as a function of antibody-antigen complex absorption (OD₄₅₀) at 3 serum dilutions of immune sera obtained from 3 succesive bleeds (pre-immune, 1^{st} bleed, and final bleed) in rabbits (n=2) immunized with antigen-KLH conjugates corresponding to (A) 6-Mix-KLH 42; (B) 12-Mix-KLH 44; and (C) 18-Mix-KLH 46. In each case, the antisera were incubated on ELISA plates adsorbed with their corresponding BSA conjugate, specifically, (A) 6-Mix-BSA 41; (B) 12-Mix-BSA 43; and (C) 18-Mix-BSA 45 as described the ELISA protocol above (Example 14).

FIGS. 11D-11F depict antigen-specific IgG antibody titers from antigen-KLH conjugate-derived antibodies recovered at three successive stages of fraction, and the antibody-enriched (purified) fraction. Results are shown as a function of antibody-antigen complex absorption (OD₄₅₀) at the indicated serum dilutions obtained from the above-described antibody-enriched fractions generated against antigen-KLH conjugates corresponding to (A) 6-Mix-KLH 42; (B) 12-Mix-KLH 44; and (C) 18-Mix-KLH 46. In each case, the antisera were incubated on ELISA plates adsorbed with their corresponding BSA conjugate, specifically, (A) 6-Mix-BSA 41; (B) 12-Mix-BSA 43; and (C) 18-Mix-BSA 45.

Affinity purification of 10 mL of 3^{rd} bleed sera (in each case) yielded: 5.5 mL of a purified 6-Mix Ab solution at 2.3 mg/mL (12.7 mg purified Ab total); 5.4 mL of a purified 12-Mix Ab solution at 5.5 mg/mL (29.7 mg purified Ab total); and 5.4 mL of purified 18-Mix Ab solution at 1.7 mg/mL (9.2 mg purified Ab total).

### Example 16

### Specificity and Cross-Reactivity of Antisera to Different Antigens

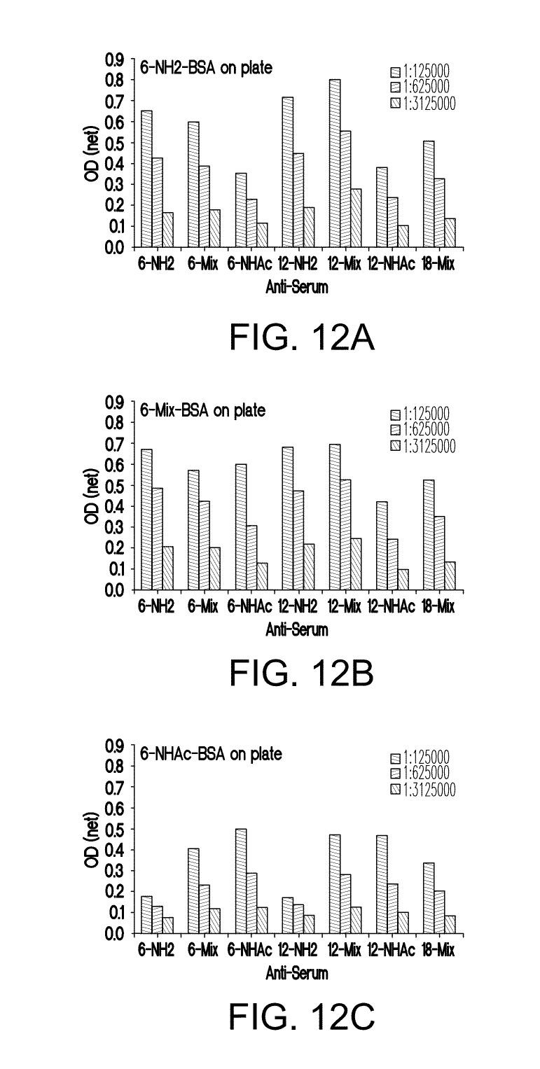

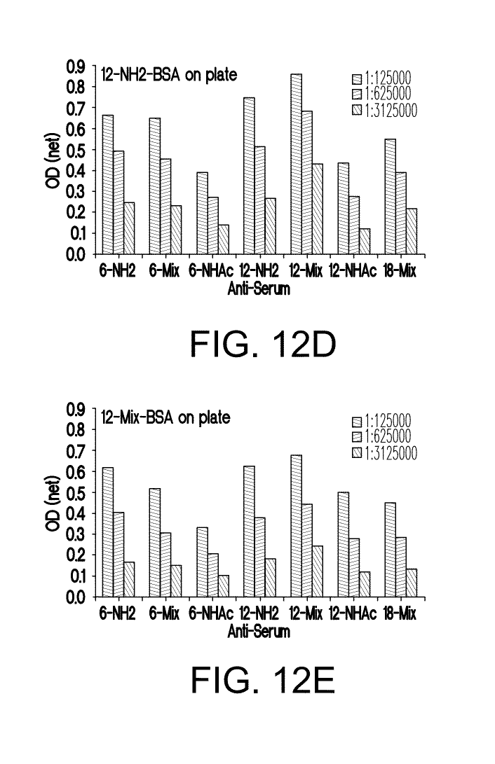

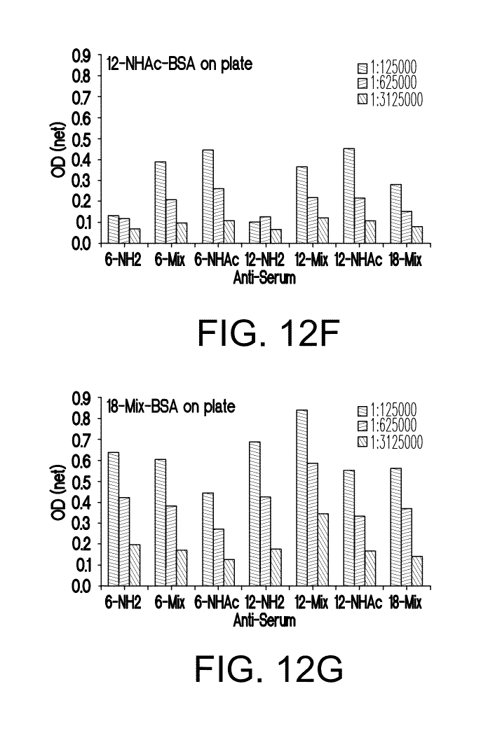

FIGS. 12A-12G depict the results of a cross-ELISA assay examining the specificity and cross-reactivity between fully non-acetylated (6-NH₂ 61, 12-NH₂ 62, 18-NH₂ 63;); mixed (6-Mix 37, 12-Mix 40, 18-Mix 34) and fully acetylated (6-NHAc 58, 12-NHAc 59, 18-NHAc 60;) oligo-β-(1→6)-glucosamines and antibodies derived therefrom. In FIGS. 12A-12G, antisera from rabbits immunized with the indicated antigen-KLH conjugates corresponding to (left to right) 6-NH₂, 6-Mix 42, 6-NHAc, 12-NH₂ 44, 12-Mix, 12-NHAc, 18-NH₂, 18-Mix 46, and 18-NHAc were incubated in each case with an ELISA plate adsorbed with a different antigen-BSA conjugate, specifically (A) 6-NH₂—BSA 64; (B) 6-Mix-BSA 41; (C) 6-NHAc—BSA 65; (D) 12-NH₂—BSA 66; (E) 12-Mix-BSA 43; (F) 12-NHAc—BSA; (G) 18-Mix-BSA 45. Results are shown as a function of antibody-antigen complex absorption (OD₄₅₀) representing the averages from of antisera from two rabbits in each case at the indicated serum dilutions, whereby total OD₄₅₀ is measured by subtracting away the background OD₄₅₀ from KLH antibodies alone. The results surprisingly showed that antibodies against the 12-Mix-KLH conjugate 44 antigen (16.7% acetylation) cross-reacted strongly with all of the antigens screened, including fully non-acetylated, mixed, and fully acetylated antigens, and typically to a greater extent than antibodies against the fully acetylated or fully aminylated oligosaccharides.

### Example 17

### Whole-Cell ELISA Experiment

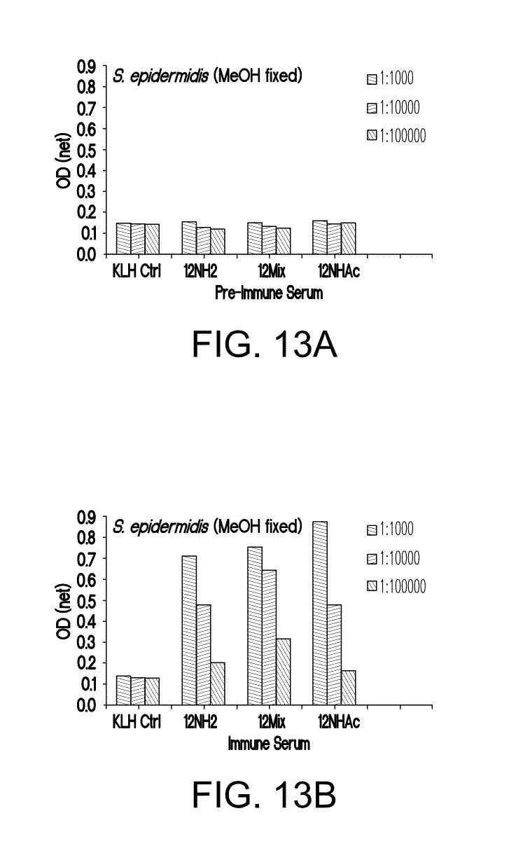

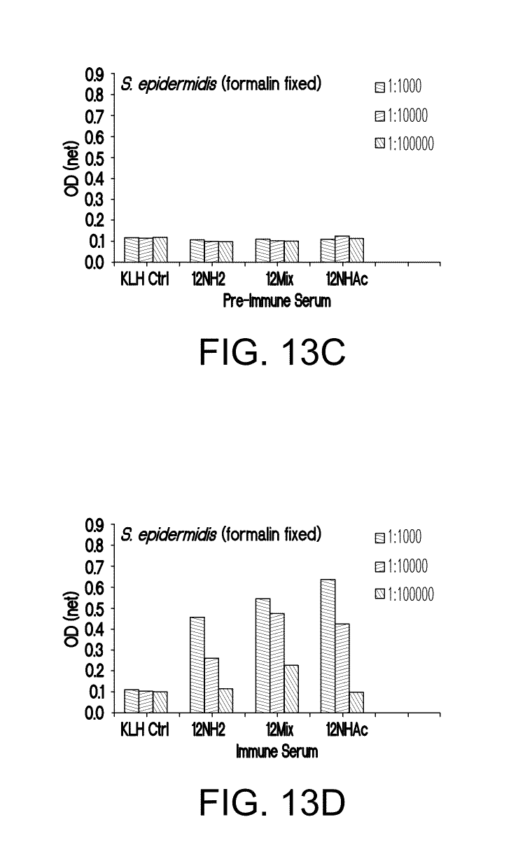

FIGS. 13A-13D depict the results of a whole-cell ELISA assay examining the binding of pre-immune sera (A, C) or immune sera (B, D) generated from rabbits immunized against (left to right) KLH control, fully non-acetylated antigen (12-NH₂) 62; mixed antigen (12-Mix) 44 and fully acetylated antigen (12-NHAc) 59 and Staphylococcus epidermidis coated onto ELISA fixed with methanol (A, B) or formalin (C, D). Results are shown as a function of antibody-antigen complex absorption (OD₄₅₀) at the indicated serum dilutions. The results surprisingly showed that at the higher dilutions, antibodies against the poorly acetylated 12-Mix-KLH conjugate 44 antigen (16.7% acetylation) reacted more strongly to S. epidermidis than antibodies against the fully non-acetylated (12-NH₂) or fully acetylated (12-NHAc) antigens even though the PNAG/dPNAG from natural S. epidermidis isolates are highly acetylated.

### Example 18

### Opsonophagocytic Assay

The opsonophagocytic (bacterial killing) activity of serum samples was determined in an assay using S. aureus ATCC strain 25904 in the presence of phagocytic cells and complement. Freshly isolated human PMN's were used as the effector cells in this assay. PMN's were isolated according to standard protocols known in the art. Briefly, approximately 60 mL of whole human blood was collected into 8×10-mL EDTA Vacutainer tubes. The blood from each tube was removed and carefully layered over approximately 15-mL of PMN separation medium in each of 4×50-mL conical tubes. The mixture was centrifuged at 450×g for 35 minutes at room temperature. The PNM layers from each tube were withdrawn and combined in a single tube, diluted with one volume of 0.5× sterile saline and mixed gently. The suspension was centrifuged at 450×g for 10 minutes at room temperature, and the supernatant was removed and discarded. The PMN pellet was resuspended in 5 mL of sterile saline and centrifuged again at 400-450×g for 10 minutes at room temperature. The PMN pellet was resuspended in a volume of SMEM medium to give 5×10⁷ PMN's per mL (PMN's were counted using a hemocytometer using standard protocols).

Approximately 50 ul of a stock solution of target bacteria were grown on tryptic soy agar plates with 5% sheep red blood cells (blood agar plates) and incubated overnight at 36-38° C. The bacterial lawn was transferred to a sterile 50 ml conical containing 30 mls of tryptic soy broth with 1% (w/v) glucose. The bacteria were grown in a shaking water batch set for 40 strokes per minute at 36-38° C. for 2-3 hours. The bacterial suspension was adjusted to a % T of 72-75% (1 cm light path) and 2.7-3.0 ul of this suspension was mixed with 1.4 mls of TSB for a final concentration of approximately 5-6×10⁴ cfu/ml.

Antisera raised to the antigen-carrier conjugates or antibody control solutions were serially diluted and 40 ul of each dilution of each antiserum or control antibody were added to each well of a 96-well round bottom plate (Nunc 163320 or equivalent). Antibody dilutions were made in DMEM/F12 medium buffered with 10 mM HEPES to maintain a pH of 7.2-7.6.

Forty ul of the PMN cell suspension was added to each well of the 96-well assay plate followed by addition of 10 ul of complement (at different dilutions) per well. The complement was derived from human serum treated with protein A and protein L to extract inherent antibodies reactive with the target bacteria.

The OP reaction was initiated with the addition of 10 ul of the bacterial suspension to each well and the plate was incubated at 36-37° C. in a shaking incubator at ˜100 rpm for 30-40 minutes.

Following addition of the bacteria to the assay plate, the reagents were mixed by rapid pipetting up and down 20-25 times using a multichannel pipettor set at 10 ul. After mixing a sample was removed from each well, diluted 20-fold in water containing 0.1% BSA and 0.01% Tween20. These samples were designated the T₀ samples and 100 ul of each T₀ sample was transferred to a blood agar plate, allowed to dry, inverted and incubated overnight at 36-37° C.

After transfer of the T₀ samples the assay plate was incubated at 36-37° C. in an orbital shaking incubator at 250-300 rpm for an additional 90 minutes. At the end of this incubation period samples were taken from each well, diluted and plated as described above (T₉₀ samples).

Assay controls included PMN's alone, PMN's with complement and reference antibody. The percentage of bacterial killing was calculated using the formula:

$$\frac{( Number of colonies {T}_{0} - Number of colonies {T}_{90} )}{( Number of Colonies {T}_{0} )} * 100$$

The results are shown below:

TABLE
C′ mediated killing of S. epidermidis (ATCC 25984)(Anti-serum @ 1:40)

Bacterial Death (%)

| | Pre-immune | 3^{rd} bleed |
|---|---|---|
| Positive control | 0 | 5 |
| Carrier | 50 | 70 |
| Antiserum 1 (Ag8-Carrier) | 30 | 80 |
| Antiserum 2 (Ag9-Carrier) | 0 | 70 |

TABLE
C′ mediated killing of S. aureus Newman (Anti-serum @ 1:100)

Bacterial Death (%)

| | Pre-immune | 3^{rd} bleed |
|---|---|---|
| Ag8 | 30 | 70 |
| Ag9 | 0 | 50 |
| Ag10 | 20 | 60 |
| Ag10 | 10 | 80 |

### Example 19

### In Vivo Challenge Protocol

Five week old female Crl:CD-1® Swiss outbred mice were acclimated for 7 days prior to study start. Mice were randomized into study groups (n=10 per group) the day before initial immunization. Test articles were reconstituted using sterile saline to the appropriate dosing concentration (10 μg glycan equivalent of test article/100 μL solution). Each dose was mixed with an equal volume (100 μL) of CFA or IFA to form a stable emulsion. On Day 0, mice were administered a single subcutaneous (SC) treatment of the appropriate test or control article at a volume of 100 μL. Prime immunizations were followed by two boost immunizations on Days 11 and 22. A separate group of untreated mice (untreated control) were not be vaccinated.

On Day 29, each mouse was challenged via intravenous tail injection (IV) route with Staphylococcus aureus Newman strain at a concentration of approximately 4×10⁹ CFU/mL in a dose volume of 0.2 mL. Bacteria inoculation suspensions were prepared by harvesting isolated colonies seeding 50-mL of fresh Trypticase Soy Broth (TSB). The culture was incubated (37° C.) with shaking for approximately 3-5 hours, washed and resuspended. The concentration in the final culture was adjusted to 4.0×10⁹ CFU/mL dose using a spectrophotometer (Target OD₆₂₀=3.0). The concentration was verified using the dilution plate count method.

On Days −3, 10, 21 and 28 mice were bled via retro-orbital sinus (approx. 0.2 mL) into serum separator tubes for processing of sera (stored frozen at −20° C.). On Days 30, 31 and 32, mice were bled in the sub-mandibular region (approx. 0.1 mL) onto solid media for bacteremia analysis. All surviving animals will be euthanized via CO₂ asphyxiation on Day 36. The percentage of survival and mortality for each group was determined and microbiological analyses of blood were expressed as ±bacteremia.

The results are shown below:

TABLE
Day 7 Survival

| | | % survival |
|---|---|---|
| Vaccine group | dPNAG1-carrier (Ag8-Carrier) | 88 |
| | dPNAG2-carrier (Ag9-Carrier) | 78 |
| | PNAG-carrier (Ag10-Carrier) | 60 |
| Control group | Carrier alone | 60 |
| | untreated | 50 |

It is intended that the foregoing detailed description be regarded as illustrative rather than limiting, and that it be understood that it is the following claims, including all equivalents, that are intended to define the spirit and scope of this invention.

1. A homogenous composition consisting essentially of a synthetic oligosaccharide 1a 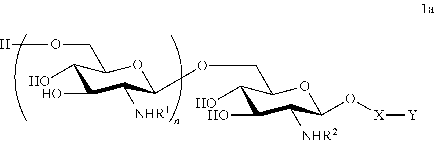
wherein R¹ and R² are independently selected from H or C(O)CH₃; n is an integer of at least 3, X is a bond or a linker, and Y is H or a carrier;
wherein at least one R¹ or R² in the oligosaccharide is H and at least another one is C(O)CH₃; wherein each occurrence of R¹ can be the same or different.

2. The homogenous composition of claim 1, wherein n is an integer between 3 and 50.

3. The homogenous composition of claim 1, wherein the synthetic oligosaccharide has one of the structures of compounds 1045 to 1106 shown in Table 3, one of the structures of compounds 1107 to 1232 shown in Table 4, one of the structures of compounds 1233 to 1486 shown in Table 5, one of the structures of compounds 1487 to 1996 shown in Table 6, one of the structures of compounds 1997 to 3018 shown in Table 7, one of the structures of compounds 3019 to 5064 shown in Table 8, or one of the structures of compounds 5065 to 9158 shown in Table 9.

4. The homogenous composition of any one of claim 1, wherein n is an integer between 4 and 17.

5. The homogenous composition of claim 1, wherein at least 3 of the R¹ and R² groups are C(O)CH₃.

6. The homogenous composition of claim 1, wherein at least 3 of the R¹ and R² groups are H.

7. The homogenous composition of claim 1, wherein the synthetic oligosaccharide has a first monosaccharide unit wherein R¹ is H and a second monosaccharide unit wherein R¹ or R² is C(O)CH₃, and wherein said second monosaccharide unit is located three monosaccharide units from the first monosaccharide unit.

8. The homogenous composition of claim 1, wherein
(a) the number of occurrences for R¹ being H in every third monosaccharide unit in the oligosaccharide sequence is 0, 1, or 2; or
(b) the number of occurrences for R¹ and R² being H in every third monosaccharide unit in the oligosaccharide sequence is 0, 1, or 2, provided that the monosaccharide unit carrying R² is in said third position.

9. The homogenous composition of claim 1, wherein
(a) n is 5 and 3 of the R¹ or R² groups are C(O)CH₃;
(b) n is 6 or 7 and at least 4 of the R¹ or R² groups are C(O)CH₃;
(c) n is 8 or 9 and at least 5 of the R¹ or R² groups are C(O)CH₃; or
(d) n is 10 or 11 and at least 6 of the R¹ or R² groups are C(O)CH₃.

10. The homogenous composition of claim 1, wherein:
(a) n is 5 and 3 of the R¹ or R² groups are H;
(b) n is 6 or 7 and at least 4 of the R¹ or R² groups are H;
(c) n is 8 or 9 and at least 5 of the R¹ or R² groups are H; or
(d) n is 10 or 11 and at least 6 of the R¹ or R² groups are H.

11. The homogenous composition of claim 1, wherein the R¹ groups located in 3 sequential monosaccharide units are C(O)CH₃ or wherein two R¹ groups and the R² group located in 3 sequential monosaccharide units are C(O)CH₃.

12. The homogenous composition of claim 1, wherein the percentage of N-acetylated monosaccharide units in the oligomer is between 10% and 70%, 20% and 70%, 30% and 70%, or 40% and 60%.

13. The homogenous composition of claim 1, wherein the linker X is a bond.

14. The homogenous composition of claim 1, wherein the linker comprises a substituted or unsubstituted (C₁-C₁₀)alkylene or (C₂-C₁₀)alkenylene moiety.

15. The homogenous composition of claim 1, wherein X is —(CH₂)ₚS—, where p is an integer from the group consisting of 3, 4, 5, 6, 7, 8, 9, 10, and
(i) Y is a carrier, or
(ii) Y is H.

16. The homogenous composition of claim 1, wherein Y is a carrier selected from the group consisting of proteins, peptides, lipids, polymers, dendrimers, virosomes, and virus-like particles or combination thereof.

17. The homogenous composition of claim 16, wherein the carrier is a protein.

18. The homogenous composition of claim 17, wherein the protein is selected from the group consisting of bacterial toxoids, toxins, exotoxins, and nontoxic derivatives thereof.

19. The homogenous composition of claim 18, wherein the protein is selected from the group consisting of tetanus toxoid, tetanus toxin Fragment C, diphtheria toxoid, CRM, cholera toxoid, Staphylococcus aureus exotoxins or toxoids, Escherichia coli heat labile enterotoxin, Pseudomonas aeruginosa exotoxin A, genetically detoxified variants thereof; bacterial outer membrane proteins, serotype B outer membrane protein complex (OMPC), outer membrane class 3 porin (rPorB), porins; keyhole limpet hemocyanine (KLH), hepatitis B virus core protein, thyroglobulin, albumins, and ovalbumin; pneumococcal surface protein A (PspA), pneumococcal adhesin protein (PsaA); purified protein derivative of tuberculin (PPD); transferrin binding proteins, peptidyl agonists of TLR-5; and derivatives and/or combinations of the above carriers.

20. The homogenous composition of claim 19, wherein the protein is selected from the group consisting of CRM 197, Neisseria meningitides, bovine serum albumin (BSA), human serum albumin (HSA), poly(lysine:glutamic acid), flagellin of motile bacteria, and derivatives and/or combinations thereof.

21. The homogenous composition of claim 20, wherein the carrier is selected from the group consisting of tetanus toxoid, CRM 197, and OMPC.

22. A composition comprising a homogenous composition of claim 1 and a pharmaceutically acceptable vehicle.

23. The composition of claim 22, further comprising an adjuvant.

24. The composition of claim 22, wherein the homogenous composition is present in an effective amount to stimulate an immune response.

25. The composition of claim 24, wherein the immune response is an antigen-specific immune response.

26. The composition of claim 22, wherein the homogenous composition confers immunity against Staphylococcus, Escherichia coli; Yersinia species (spp.), Bordetella spp., Aggregatibacter actinomycetemcomitans, Actinobacillus pleuropneumoniae; Acinetobacter spp.; Burkholderia spp.; Stenatrophomonas maltophilia, Klebsiella spp., and Shigella spp.

27. The composition of claim 26, wherein the homogenous composition confers immunity against Y. pestis, Y. pseudotuberculosis, Y. entercolitica, B. pertussis, B. bronchiseptica, or B. parapertussis.

28. An antibody preparation against a homogenous composition of claim 1.

29. A method of treating or preventing a Staphylococcus infection in a patient in need thereof comprising administering an effective amount for inducing an immune response against Staphylococcus of a homogenous composition of claim 1 or an antibody thereto.

30. A method for producing antibodies comprising:
(a) administering to a subject an effective amount of a homogenous composition of claim 1, for producing antibodies specific for PNAG or dPNAG;
(b) isolating antibodies from the subject.

31. A multi-valent vaccine comprising a homogenous composition of claim 1.
